(19) United States
(12) Patent Application Publication
(10) Pub. No.: US 2025/0170141 A1
(43) Pub. Date: May 29, 2025

(54) MRGPRX2 MODULATORS AND RELATED METHODS OF TREATMENT

(71) Applicant: Escient Pharmaceuticals, Inc., San Diego, CA (US)

(72) Inventors: Marion Lanier, San Diego, CA (US); Brandon Selfridge, San Diego, CA (US); Liming Huang, San Diego, CA (US); Adam Yeager, San Diego, CA (US); Marcus Boehm, San Diego, CA (US); Esther Martinborough, San Diego, CA (US)

(21) Appl. No.: 18/907,256

(22) Filed: Oct. 4, 2024

Related U.S. Application Data
(60) Provisional application No. 63/588,638, filed on Oct. 6, 2023.

(51) Int. Cl. A61K 31/549; C07D 285/24; C07D 417/04; C07D 417/12

(52) U.S. Cl. CPC A61K 31/549; C07D 285/24; C07D 417/04; C07D 417/12

(57) ABSTRACT

Disclosed herein are compounds having the structure of Formula (I):

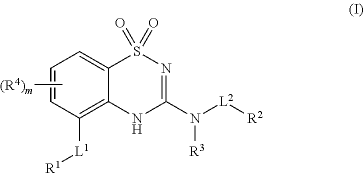

or a pharmaceutically acceptable salt, solvate, hydrate, isomer, tautomer, racemate, or isotope thereof, wherein L¹, L², R¹, R², R³, R⁴, and m are as defined herein. Pharmaceutical compositions comprising them, processes for preparing them, and uses of them to treat or prevent diseases, disorders, and conditions are also provided. The compounds are modulators of MRGPRX2 or the MRGPRX2 ortholog and are useful in the treatment of diseases, disorders, and conditions related to MRGPRX2 or the MRGPRX2 ortholog activity.

### BACKGROUND

### Technical Field

The invention relates to modulators of the Mas-related G-protein coupled receptor X2, to products containing the same, as well as to methods of their use and preparation.

### Description of the Related Art

Mas-related G-protein receptors (MRGPRs) are a group of orphan receptors with limited expression in very specialized tissues. Very little is known about the function of most of these receptors. In humans, there are eight members in the MRGPR family of receptors. Of these, four members (MRGPRD, E, F and G) have readily identifiable orthologs in mammals.

The other four receptors (MRGPR X1, X2, X3 and X4) have counterparts in higher species including dogs and horses, but they do not have a single corresponding ortholog in rodents.

Mas-related G protein-coupled receptor X2 (MRGPRX2) is an orphan, seven transmembrane G protein-coupled receptor that is almost exclusively expressed on connective tissue mast cells. MRGPRX2 belongs to a G protein-coupled receptor subfamily X, comprised of four members X1-X4, specific to humans and primates. MRGPRX2 is a low affinity promiscuous receptor for cyclic and polybasic structure ligands that mediates mast cell degranulation in response to multiple endogenous and exogenous stimuli.

Mast cells constitute an integral part of the human immune system. They are important modulators of inflammatory and physiological processes. MRGPRX2 receptor plays a pivotal role in itch, allergy and inflammation. Activation of the receptor by neuropeptides, antimicrobial host defense peptides as well as numerous FDA-approved drugs leads to mast cell degranulation and release of inflammatory mediators through immunoglobulin-independent pathway (M. Thapaliya, et al., Curr. Allergy Asthma Rep., 2021, 21(1), 3).

Activation of MRGPRX2 receptor drives non-histaminergic itch in chronic refractory pruritus and MRGPRX2 has also been implicated in senile itch (A. He, et al. Biomed. Res. Int., 2017, 4790810; J. Meixiong, et al., Immunity, 2019, 50(5), 1163-71). There is an increased MRGPRX2 gene expression on mast cells in the skin of patients with severe chronic urticaria (hives) (H. Ali, J Immunobiol., 2016, 1(4), 115; D. Fujisawa, et al., J. Allergy Clin. Immunol. 2014, 134(3), 622-33). Additionally, activation of MRGPRX2 by elevated levels of proadrenomedullin N-terminal 20 peptide (PAMP1-20) in the skin of patients with allergic contact dermatitis (ACD) leads to intensely itchy eczematous skin rash (J. Meixiong, et al., Immunity, 2019, 50(5):1163-71). MRGPRX2 is also involved in the pathogenesis of acne rosacea where dysregulation of the host defense mechanism due to excessive LL-37 antimicrobial peptide production leads to enhanced mast cell activation through MRGPRX2 (H. Ali, Adv. Immunol., 2017, 136, 123-62). MRGPRX2 is also implicated in systemic mastocytosis and in neurogenic inflammation, pain and itch. Substance P released from nerve endings and directly from mast cells in sickle cell anemia patients activates mast cells via MRGPRX2 causing painful crisis (H. Subramanian, et al., J. Allergy Clin. Immunol., 2016, 138(3), 700-10).

The role of MRGPRX2 in mast cell biology is further supported by the fact that naturally occurring missense MRGPRX2 variants: G165E, D184H, W243R, and H259Y inhibit mast cell degranulation in response to endogenous neuropeptides and drugs (I. Alkanfari, et al., J. Immuol., 2018, 201(2), 343-49).

Taken together, these findings suggest that MRGPRX2 plays a critical role in itch, pain, and inflammation. Potential disease indications for MRGPRX2 antagonist encompass chronic urticaria and pruritus (hives/itch), acne rosacea, and systemic mastocytosis. These clinical indications present high unmet medical need, particularly in antihistamine-refractory patients. Therefore, targeting of MRGPRX2 receptor can be useful in the clinical treatment of mast-cell mediated diseases.

There remains a need for new compounds that are effective as modulators, antagonists or inhibitors of MRGPRX2.

### BRIEF SUMMARY

This invention is based, in part, on the identification of MRGPRX2 or MRGPRX2 ortholog modulator compounds. Among rodent orthologs, mouse mrgprb2 and rat mrgprb3 correspond functionally to human MRGPRX2 in mast cells. MRGPRX2 and its ortholog receptors mediate disorders including pseudo-allergic drug reactions, chronic itch (e.g., pruritus), inflammation disorders, pain disorders, a cancer associated condition, skin disorders, wound healing, cardiovascular disease, and lung inflammation/COPD. In one embodiment, expression of MRGPRX2 and its orthologs is largely restricted to mast cells. Mast cells are innate immune cells that primarily reside at sites exposed to the external environment, such as the skin, oral/gastrointestinal mucosa and respiratory tract. Mast cells express numerous receptors that respond to mechanical and chemical stimuli. Upon activation, classically by IgE, mast cells release pre-formed mediators from granules (e.g., histamine, proteases, and heparin) and newly synthesized mediators (e.g., thromboxane, prostaglandin D2, leukotriene C4, tumor necrosis factor alpha, eosinol chemotactor factor, and platelet-activating factor) that elicit allergic and inflammatory responses. Histamine dilates post-capillary venules, activates the endothelium, and increases blood vessel permeability. This causes local edema, warmth, redness, and chemotaxis of other inflammatory cells to the site of release. Histamine also contributes to neuronal sensitization that leads to pain or itch. MRGPRX2 and its orthologs mediate immunoglobulin E (IgE) independent activation of mast cells. MRGPRX2 and its orthologs are receptors for (or sensitive to activation by) various ligands, including basic secretagogues (small cationic molecules), certain drugs (e.g., cationic peptidergic drugs), neuropeptides, and antimicrobial peptides, and thus are important for non-IgE mediated pseudo-allergic reactions, inflammation, pain, and itch conditions. Mast cells may also contribute to the progression of autoimmune disorders by promoting chronic inflammation in the local tissue microenvironment and ultimately polarizing toward a Tₕ17 immune response. Thus, modulating MRGPRX2 or MRGPRX2 orthologs allows for treatment of autoimmune diseases, pseudo-allergic drug reactions, pain, itch, and inflammatory disorders such as inflammatory bowel disease, urticaria, sinusitis, asthma, rosacea, endometriosis, and other MRGPRX2 or MRGPRX2 ortholog dependent conditions as explained in more detail below.

In one embodiment is provided a method of treating a MRGPRX2 or a MRGPRX2 ortholog dependent condition by administering to a subject in need thereof an effective amount of the pharmaceutical composition of the modulator compounds of the present invention.

Accordingly, in an embodiment, is provided a compound having structure (I):

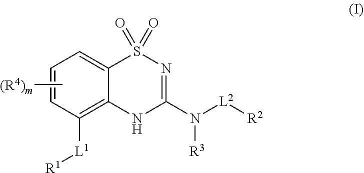

or a pharmaceutically acceptable salt, isomer, hydrate, solvate, or isotope thereof, wherein L¹, L², R¹, R², R³, R⁴, and m are as defined herein.

In other embodiments, compounds are provided having formula (II), (III), (III-A), or (III-B) as defined herein, or a pharmaceutically acceptable salt, isomer, hydrate, solvate, or isotope thereof.

In yet other embodiments, pharmaceutical compositions are provided comprising a carrier or excipient and a compound having structure (I), or a pharmaceutically acceptable salt, isomer, hydrate, solvate, or isotope thereof.

In another embodiment, methods are provided for treating an MRGPRX2 or a MRGPRX2 ortholog dependent condition by administering to a subject in need thereof an effective amount of a compound having structure (I), or a pharmaceutically acceptable salt, isomer, hydrate, solvate or isotope thereof.

In some embodiments, the MRGPRX2 or a MRGPRX2 ortholog dependent condition is one or more of a pseudo-allergic reaction, itch associated condition, a pain associated condition, a cancer associated condition, an inflammation-associated condition, or an autoimmune disorder.

In another embodiment, compounds are provided having one or more of the structures disclosed herein, or a pharmaceutically acceptable salt, isomer, hydrate, solvate or isotope thereof.

### DETAILED DESCRIPTION

As mentioned above, the invention relates to modulators of MRGPRX2 (or MRGPRX2 ortholog), to products containing the same, as well as to methods of their use and preparation. This invention is based, in part, on the identification of MRGPRX2 modulator compounds and MRGPRX2 ortholog modulator compounds. MRGPRX2 and MRGPRX2 orthologs are expressed in mast cells. MRGPRX2 and MRGPRX2 orthologs are receptors for (or sensitive to activation by) a diverse group of ligands, including basic secretagogues, certain drugs, neuropeptides, antimicrobial peptides, and thus are important for pseudo-allergic reactions, itch, pain, or inflammatory disorders upon exposure.

MRGPRs appear to be sensory receptors that recognize their external environment to exogenous or endogenous signals/chemicals. These receptors likely respond to multiple chemical ligands/agonists. For example, MRGPRX2 recognizes Compound 48/80, Substance P, Mastoparan, Icatibant, Ciprofloxacin, and atracurium as agonist signals. In certain embodiments, molecules of this invention modulate MRGPRX2 by functioning as inverse agonists that are capable of blocking multiple chemical entities, and/or as competitive antagonists that can specifically block individual ligands. In one embodiment, such modulations are selective against other MRGPRs, such as MRGPR X1, X3, X4 and/or D.

Unless defined otherwise, all technical and scientific terms used herein have the same meaning as is commonly understood by one of skill in the art to which the claimed subject matter belongs. It is to be understood that the detailed description is exemplary and explanatory only and is not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

As used herein, ranges and amounts can be expressed as “about” a particular value or range. About also includes the exact amount. Hence “about 100 μL” means “about 100 μL” and also “100 μL.” In some embodiments, about means within 5% of the value. Hence, “about 100 μL” means 95-105 μL. In some embodiments, about means within 4% of the value. In some embodiments, about means within 3% of the value. In some embodiments, about means within 2% of the value. In some embodiments, about means within 1% of the value. Generally, the term “about” includes an amount that would be expected to be within experimental error.

### Compounds

One embodiment provides a compound having the structure of Formula (I):

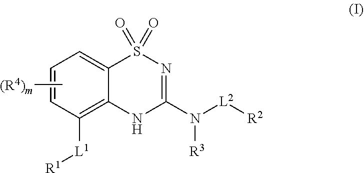

or a pharmaceutically acceptable isomer, racemate, hydrate, solvate, isotope, or salt thereof, wherein:
L¹ is —(CR′R″)_{q}—, —(CR′R″)ᵤ—CR′═CR′—(CR′R″)ᵥ—, —(CR′R″)ᵤ—C≡C—(CR′R″)ᵥ—, —(CR′R″)ᵤ—O—(CR′R″)ᵥ—, —(CR′R″)ᵤ—NR′—(CR′R″)ᵥ—, —(CR′R″)ᵤ—C(O)—(CR′R″)ᵥ—;
L² is —(CR^{a}R^{b})_{w}—;
R¹ is C₁₋₆ alkyl, carbocycle or heterocycle, wherein R¹ is substituted with —(R⁵)ₙ;
R² is halo, C₁₋₆ alkyl, carbocycle or heterocycle, wherein R² is substituted with —(R⁶)ₚ;
R³ is H or C₁₋₆ alkyl, or R³ and one R⁶, together with the atoms to which they are attached, form a ring;
R⁴ is, at each occurrence, independently halo, —CN, —OR⁷, —N(R⁷)₂, C₁₋₆ alkyl, C₁₋₆ haloalkyl;
R⁵ is, at each occurrence, independently halo, —CN, —OR⁷, —N(R⁷)₂, —S(O)₂—R⁷, C₁₋₆ alkyl, C₁₋₆ haloalkyl, carbocycle, or heterocycle;
R⁶ is, at each occurrence, independently halo, —CN, —OR⁷, —N(R⁷)₂, —SR⁷, —C(O)OR⁷, —N(R⁸)C(O)R⁷, —N(R⁸)C(O)OR⁷, —C(O)N(R⁸)₂, —S(O)₂—R⁷, —C₁₋₆ alkyl-R⁷, C₁₋₆ haloalkyl, carbocycle substituted with —(R⁷)ₓ, or heterocycle substituted with —(R⁷)ₓ;
R⁷ is, at each occurrence, independently H, —OR⁸, —N(R⁸)₂, —C(O)OR⁸, —NHC(O)OR⁸, —C₁₋₆ alkyl-R⁸, C₁₋₆ haloalkyl, carbocycle substituted with —(R⁸)_{y}, or heterocycle substituted with —(R⁸)_{y};
R⁸ is, at each occurrence, independently H, —OR, —NR₂, —C₁₋₆ alkyl-R, carbocycle substituted with —(R)_{z}, or heterocycle substituted with —(R)_{z};
R′ and R″ are, at each occurrence, independently H, halo, cyano, hydroxy, amino, C₁₋₆ alkyl, or C₁₋₆ alkenyl, or, independently, at each occurrence of R′ and R″ attached to the same carbon atom, R′ and R″, together with the carbon to which they are attached, form a cycloalkyl;
R^{a} and R^{b} are, at each occurrence, independently H, halo, cyano, hydroxy, amino, or C₁₋₆ alkyl or, independently, at each occurrence of R^{a} and R^{b} attached to the same carbon atom, R^{a} and R^{b}, together with the carbon to which they are attached, form a cycloalkyl;
R is, at each occurrence, independently H, halo, —CN, —OH, —NH₂, or C₁₋₆ alkyl;
m is 0-5;
n is 0-5;
p is 0-5
q is 1-3;
u is, at each occurrence, independently 0-3;
v is, at each occurrence, independently 0-3;
w is 0-3;
x is 0-3;
y is 0-3; and
z is 0-3;
   wherein R² is carbocycle or heterocycle when L¹ is —C(R′R″)_{q}—.

“Alkyl” means a saturated or unsaturated straight chain or branched alkyl group having from 1 to 8 carbon atoms, in some embodiments from 1 to 6 carbon atoms, in some embodiments from 1 to 4 carbon atoms, and in some embodiments from 1 to 3 carbon atoms. Examples of saturated straight chain alkyl groups include, but are not limited to, methyl, ethyl, n-propyl, n-butyl, n-pentyl-, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. An unsaturated alkyl includes alkenyl and alkynyl as defined below.

“Alkenyl” means a straight chain or branched alkenyl group having from 2 to 8 carbon atoms, in some embodiments from 2 to 6 carbon atoms, in some embodiments from 2 to 4 carbon atoms, and in some embodiments from 2 to 3 carbon atoms. Alkenyl groups are unsaturated hydrocarbons that contain at least one carbon-carbon double bond. Examples of lower alkenyl groups include, but are not limited to, vinyl, propenyl, butenyl, pentenyl, and hexenyl.

“Alkynyl” means a straight chain or branched alkynyl group having from 2 to 8 carbon atoms, in some embodiments from 2 to 6 carbon atoms, in some embodiments from 2 to 4 carbon atoms, and in some embodiments from 2 to 3 carbon atoms. Alkynyl groups are unsaturated hydrocarbons that contain at least one carbon-carbon triple bond. Examples of alkynyl groups include, but are not limited to, ethynyl, propynyl, butynyl, pentynyl, and hexynyl.

“Halo” or “halogen” refers to fluorine, chlorine, bromine, and iodine.

“Hydroxy” refers to —OH.

“Cyano” refers to —CN.

Amino refers to —NH₂, —NHalkyl or N(alkyl)₂, wherein alkyl is as defined above. Examples of amino include, but are not limited to —NH₂, —NHCH₃, —N(CH₃)₂, and the like.

“Haloalkyl” refers to alkyl as defined above with one or more hydrogen atoms replaced with halogen. Examples of lower haloalkyl groups include, but are not limited to, —CF₃, —CHF₂, and the like.

“Alkoxy” refers to alkyl as defined above joined by way of an oxygen atom (i.e., —O-alkyl). Examples of alkoxy groups include, but are not limited to, methoxy, ethoxy, n-propoxy, n-butoxy, isopropoxy, sec-butoxy, tert-butoxy, and the like.

“Haloalkoxy” refers to haloalkyl as defined above joined by way of an oxygen atom (i.e., —O-haloalkyl). Examples of lower haloalkoxy groups include, but are not limited to, —OCF₃, and the like.

“Cycloalkyl” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring do not give rise to aromaticity. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

“Aryl” groups are cyclic aromatic hydrocarbons that do not contain heteroatoms. Representative aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like). In one embodiment, aryl is phenyl or naphthyl, and in another embodiment aryl is phenyl.

“Carbocycle” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring may give rise to aromaticity. In one embodiment, carbocycle includes cycloalkyl as defined above. In another embodiment, carbocycle includes aryl as defined above.

“Heterocycle” refers to aromatic and non-aromatic ring moieties containing 3 or more ring members, of which one or more is a heteroatom such as, but not limited to, N, O, S, or P. In some embodiments, heterocyclyl include 3 to 20 ring members, whereas other such groups have 3 to 15 ring members. At least one ring contains a heteroatom, but every ring in a polycyclic system need not contain a heteroatom. For example, a dioxolanyl ring and a benzdioxolanyl ring system (methylenedioxyphenyl ring system) are both heterocyclyl groups within the meaning herein.

Heterocyclyl groups also include fused ring species including those having fused aromatic and non-aromatic groups. A heterocyclyl group also includes polycyclic ring systems containing a heteroatom such as, but not limited to, quinuclidyl, and also includes heterocyclyl groups that have substituents, including but not limited to alkyl, halo, amino, hydroxy, cyano, carboxy, nitro, thio, or alkoxy groups, bonded to one of the ring members. A heterocyclyl group as defined herein can be a heteroaryl group or a partially or completely saturated cyclic group including at least one ring heteroatom. Heterocyclyl groups include, but are not limited to, pyrrolidinyl, furanyl, tetrahydrofuranyl, dioxolanyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups.

In one embodiment, heterocyclyl includes heteroaryl.

“Heteroaryl” refers to aromatic ring moieties containing 5 or more ring members, of which, one or more is a heteroatom such as, but not limited to, N, O, and S. Heteroaryl groups include, but are not limited to, groups such as pyrrolyl, pyrazolyl, pyridinyl, pyridazinyl, pyrimidyl, pyrazyl, pyrazinyl, pyrimidinyl, thienyl, triazolyl, tetrazolyl, triazinyl, thiazolyl, thiophenyl, oxazolyl, isoxazolyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl, and 2,3-dihydro indolyl.

In one embodiment is provided a compound of Formula (I), or a pharmaceutically acceptable isomer, racemate, hydrate, solvate, isotope, or salt thereof, wherein R¹ is carbocycle or heterocycle.

In one embodiment, R¹ is heterocycle. In one embodiment, R¹ is heterocycloalkyl. In one embodiment, R¹ is aziridinyl, oziranyl, thiranyl, azetidinyl, 1,2-dihydroazotyl, oxetanyl, 2H-oxetyl, thietanyl, 2H-thietyl, pyrrolidinyl, 4,5-dihydro-1H-imidazolyl, imidazolinyl, pyrazolinyl, tetrahydrofuranyl, thiolanyl, piperidinyl, piperazinyl, 2H-pyranyl, 3,4-dihydro-2H-pyranyl, tetrahydro-2H-pyranyl, 1,4-dioxanyl, morpholinyl, thianyl, 1,4-dithianyl, 2H-thiopyranyl, azepanyl, 1,4-diazepanyl, oxepanyl, or thiepanyl. In one embodiment, R¹ is tetrahydro-2H-pyranyl.

In one embodiment, R¹ is heteroaryl. In one embodiment, R¹ is pyrrolyl, imidazolyl, pyrazolyl, 1,2,3-triazolyl, 1,2,4-triazolyl, tetrazolyl, furanyl, oxazolyl, isoxazolyl, 1,2,3-oxadiazolyl, 1,3,4-oxadiazolyl, 1,2,4-oxadiazolyl, 1,2,3,4-oxatriazolyl, thiophenyl, thiazolyl, isothiazolyl, 1,2,3-thiadiazolyl, 1,2,3,4-thiatriazolyl, pyridinyl, pyrazinyl, pyrimidinyl, pyridazinyl, 1,3,5-triazinyl, indolyl, benzimidazolyl, indazolyl, benzotriazolyl, 1H-pyrrolo[3,2-b]pyridinyl, 1H-pyrrolo[3,2-c]pyridinyl, pyrrolo[2,3-c]pyridinyl, pyrrolo[2,3-b]pyridinyl, [1,2,3]triazolo[4,5-b]pyridinyl, 7H-pyrrolo[2,3-d]pyrimidinyl, 5H-pyrrolo[3,2-d]pyrimidinyl, 7H-purinyl, indolizinyl, pyrrolo[1,2-a]pyrimidinyl, pyrrolo[1,2-a]pyrazinyl, pyrrolo[1,2-c]pyriminyl, pyrrolo[1,2-b]pyrdazinyl, imidazo[4,5-b]pyridinyl, pyrazolo[1,5-a]pyridinyl, imidazo[1,5-b]pyridazinyl, imidazo[1,2-a]pyridinyl, imidazo[1,2-b]pyridazinyl, imidazo[1,2-c]pyrimidinyl, imidazo[1,2-a]pyrazinyl, imidazo[1,2-a]pyrimidinyl, [1,2,4]triazolo[4,3-a]pyridinyl, [1,2,3]triazolo[1,5-a]pyridinyl, quinolinyl, isoquinolinyl, cinnolinyl, quinazolinyl, quinoxalinyl, phthalazinyl, pyrido[2,3-b]pyrazinyl, pteridinyl, pyrido[3,4-d]pyridazinyl, 1,5-naphtyridinyl, 1,8-natphyridinyl, 9H-carbazolyl, benzoxazolyl, dibenzofuranyl, benzothiophenyl, or dibenzothiophenyl. In one embodiment, R¹ is pyridinyl, furanyl, thiazolyl, pyrazolyl, or indolyl.

In one embodiment, R¹ is carbocycle. In a further embodiment, R¹ is cycloalkyl. In one embodiment, R¹ is cyclopropyl, cyclobutyl, cyclopentyl, cyclohexyl, or cycloheptyl. In one embodiment, R¹ is cyclohexyl. In another embodiment, R¹ is aryl. In one embodiment, R¹ is naphthyl, tetrahydronaphthalenyl, or phenyl.

In one embodiment, R¹ is C₁₋₆ alkyl.

In one embodiment is provided a compound having the structure of Formula (II):

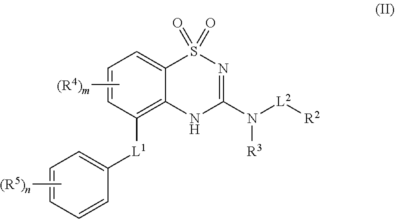

or a pharmaceutically acceptable isomer, racemate, hydrate, solvate, isotope, or salt thereof, wherein:
L¹ is —(CR′R″)_{q}, —(CR′R″)ᵤ—CR′═CR′—(CR′R″)ᵥ—, —(CR′R″)ᵤ—C≡C—(CR′R″)ᵥ—, —(CR′R″)ᵤ—O—(CR′R″)ᵥ—, —(CR′R″)ᵤ—NR′—(CR′R″)ᵥ—, —(CR′R″)ᵤ—C(O)—(CR′R″)ᵥ—; L² is —(CR^{a}R^{b})_{w};
R² is carbocycle or heterocycle, wherein R² is substituted with —(R⁶)ₚ;
R³ is H or C₁₋₆ alkyl, or R³ and one R⁶, together with the atoms to which they are attached, form a ring;
R⁴ is, at each occurrence, independently halo, —CN, —OR⁷, —N(R⁷)₂, C₁₋₆ alkyl, C₁₋₆ haloalkyl;
R⁵ is, at each occurrence, independently halo, —CN, —OR⁷, —N(R⁷)₂, —S(O)₂—R⁷, C₁₋₆ alkyl, C₁₋₆ haloalkyl, carbocycle, or heterocycle;
R⁶ is, at each occurrence, independently halo, —CN, —OR⁷, —N(R⁷)₂, —SR⁷, —C(O)OR⁷, —N(R⁸)C(O)R⁷, —N(R⁸)C(O)OR⁷, —S(O)₂—R⁷, —C₁₋₆ alkyl-R⁷, C₁₋₆ haloalkyl, carbocycle substituted with —(R⁷)ₓ, or heterocycle substituted with —(R⁷)ₓ;
R⁷ is, at each occurrence, independently H, —OR⁸, —N(R⁸)₂, —C(O)OR⁸, —NC(O)OR⁸, —C₁₋₆ alkyl-R⁸, C₁₋₆ haloalkyl, carbocycle substituted with —(R⁸)_{y}, or heterocycle substituted with —(R⁸)_{y};
R⁸ is, at each occurrence, independently H, —OR, —NR₂, —C₁₋₆ alkyl-R, carbocycle substituted with —(R)_{z}, or heterocycle substituted with —(R)_{z};
R′ and R″ are, at each occurrence, independently H, halo, cyano, hydroxy, amino, C₁₋₆ alkyl, or C₁₋₆ alkenyl, or, independently, at each occurrence of R′ and R″ attached to the same carbon atom, R′ and R″, together with the carbon to which they are attached, form a cycloalkyl;
R^{a} and R^{b} are, at each occurrence, independently H, halo, cyano, hydroxy, amino, or C₁₋₆ alkyl or, independently, at each occurrence of R^{a} and R^{b} attached to the same carbon atom, R^{a} and R^{b}, together with the carbon to which they are attached, form a cycloalkyl;
R is, at each occurrence, independently H, halo, —CN, —OH, —NH₂, or C₁₋₆ alkyl;
m is 0-5;
n is 0-5;
p is 0-5
q is 1-3;
u is, at each occurrence, independently 0-3;
v is, at each occurrence, independently 0-3;
w is 0-3;
x is 0-3;
y is 0-3; and
z is 0-3;
   wherein R² is carbocycle or heterocycle when L¹ is —C(R′R″)_{q}—.

In one embodiment is provided a compound of Formula (I) or Formula (II), or a pharmaceutically acceptable isomer, racemate, hydrate, solvate, isotope, or salt thereof, wherein R² is carbocycle or heterocycle.

In one embodiment, R² is heterocycle. In a further embodiment, R² is heterocycloalkyl. In one embodiment, R² is aziridinyl, oziranyl, thiranyl, azetidinyl, 1,2-dihydroazotyl, oxetanyl, 2H-oxetyl, thietanyl, 2H-thietyl, pyrrolidinyl, 4,5-dihydro-1H-imidazolyl, imidazolinyl, pyrazolinyl, tetrahydrofuranyl, thiolanyl, piperidinyl, piperazinyl, 2H-pyranyl, 3,4-dihydro-2H-pyranyl, tetrahydro-2H-pyranyl, 1,4-dioxanyl, morpholinyl, thianyl, 1,4-dithianyl, 2H-thiopyranyl, azepanyl, 1,4-diazepanyl, oxepanyl, thiepanyl, or 2,3-dihydrobenzo[1,4]dioxinyl, dihydrobenzofuranyl, or benzo[1,3]dioxolyl. In one embodiment, R² is tetrahydrofuranyl, 2,3-dihydrobenzo[1,4]dioxinyl, benzo[1,3]dioxolyl, or dihydrobenzofuranyl.

In another embodiment, R² is heteroaryl. In one embodiment, R² is pyrrolyl, imidazolyl, pyrazolyl, 1,2,3-triazolyl, 1,2,4-triazolyl, tetrazolyl, furanyl, oxazolyl, isoxazolyl, 1,2,3-oxadiazolyl, 1,3,4-oxadiazolyl, 1,2,4-oxadiazolyl, 1,2,3,4-oxatriazolyl, thiophenyl, thiazolyl, isothiazolyl, 1,2,3-thiadiazolyl, 1,2,3,4-thiatriazolyl, pyridinyl, pyrazinyl, pyrimidinyl, pyridazinyl, 1,3,5-triazinyl, indolyl, benzimidazolyl, indazolyl, benzotriazolyl, 1H-pyrrolo[3,2-b]pyridinyl, 1H-pyrrolo[3,2-c]pyridinyl, pyrrolo[2,3-c]pyridinyl, pyrrolo[2,3-b]pyridinyl, [1,2,3]triazolo[4,5-b]pyridinyl, 7H-pyrrolo[2,3-d]pyrimidinyl, 5H-pyrrolo[3,2-d]pyrimidinyl, 7H-purinyl, indolizinyl, pyrrolo[1,2-a]pyrimidinyl, pyrrolo[1,2-a]pyrazinyl, pyrrolo[1,2-c]pyriminyl, pyrrolo[1,2-b]pyrdazinyl, imidazo[4,5-b]pyridinyl, pyrazolo[1,5-a]pyridinyl, imidazo[1,5-b]pyridazinyl, imidazo[1,2-a]pyridinyl, imidazo[1,2-b]pyridazinyl, imidazo[1,2-c]pyrimidinyl, imidazo[1,2-a]pyrazinyl, imidazo[1,2-a]pyrimidinyl, [1,2,4]triazolo[4,3-a]pyridinyl, [1,2,3]triazolo[1,5-a]pyridinyl, quinolinyl, isoquinolinyl, cinnolinyl, quinazolinyl, quinoxalinyl, phthalazinyl, pyrido[2,3-b]pyrazinyl, pteridinyl, pyrido[3,4-d]pyridazinyl, 1,5-naphtyridinyl, 1,8-natphyridinyl, 9H-carbazolyl, benzoxazolyl, dibenzofuranyl, benzothiophenyl, or dibenzothiphenyl. In one embodiment, R² is pyridinyl, pyrimidinyl, oxazolyl, isothiazolyl, thiazolyl, isoquinolinyl, indolyl, benzo[d]oxazolyl, quinolinyl, pyrazinyl, or indazolyl. In one embodiment, R² is pyridinyl.

In one embodiment, R² is carbocycle. In a further embodiment, R² is cycloalkyl. In one embodiment, R² is cyclopropyl, cyclobutyl, cyclopentyl, cyclohexyl, or cycloheptyl. In another embodiment, R² is aryl. In one embodiment, R² is naphthyl or phenyl.

In one embodiment is provided a compound having the structure of Formula (III):

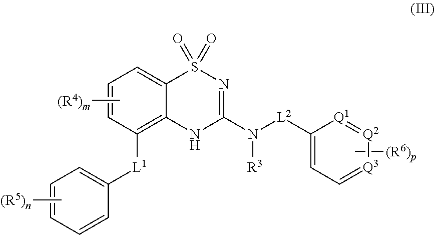

or a pharmaceutically acceptable isomer, racemate, hydrate, solvate, isotope, or salt thereof, wherein:
L¹ is —(CR′R″)_{q}—, —(CR′R″)ᵤ—CR′═CR′—(CR′R″)ᵥ—, —(CR′R″)ᵤ—C≡C—(CR′R″)ᵥ—, —(CR′R″)ᵤ—O—(CR′R″)ᵥ—, —(CR′R″)ᵤ—NR′—(CR′R″)ᵥ—, —(CR′R″)ᵤ—C(O)—(CR′R″)ᵥ—;
L² is —(CR^{a}R^{b})_{w}—;
Q¹, Q², and Q³ are, at each occurrence, independently CR⁶ or N;
R³ is H or C₁₋₆ alkyl, or R³ and one R⁶, together with the atoms to which they are attached, form a ring;
R⁴ is, at each occurrence, independently halo, —CN, —OR⁷, —N(R⁷)₂, C₁₋₆ alkyl, C₁₋₆ haloalkyl;
R⁵ is, at each occurrence, independently halo, —CN, —OR⁷, —N(R⁷)₂, —S(O)₂—R⁷, C₁₋₆ alkyl, C₁₋₆ haloalkyl, carbocycle, or heterocycle;
R⁶ is, at each occurrence, independently halo, —CN, —OR⁷, —N(R⁷)₂, —SR⁷, —C(O)OR⁷, —N(R⁸)C(O)R⁷, —N(R⁸)C(O)OR⁷, —S(O)₂—R⁷, —C₁₋₆ alkyl-R⁷, C₁₋₆ haloalkyl, carbocycle substituted with —(R⁷)ₓ, or heterocycle substituted with —(R⁷)ₓ;
R⁷ is, at each occurrence, independently H, —OR⁸, —N(R⁸)₂, —C(O)OR⁸, —NC(O)OR⁸, —C₁₋₆ alkyl-R⁸, C₁₋₆ haloalkyl, carbocycle substituted with —(R⁸)_{y}, or heterocycle substituted with —(R⁸)_{y};
R⁸ is, at each occurrence, independently H, —OR, —NR₂, —C₁₋₆ alkyl-R, carbocycle substituted with —(R)_{z}, or heterocycle substituted with —(R)_{z};
R′ and R″ are, at each occurrence, independently H, halo, cyano, hydroxy, amino, C₁₋₆ alkyl, or C₁₋₆ alkenyl, or, independently, at each occurrence of R′ and R″ attached to the same carbon atom, R′ and R″, together with the carbon to which they are attached, form a cycloalkyl;
R^{a} and R^{b} are, at each occurrence, independently H, halo, cyano, hydroxy, amino, or C₁₋₆ alkyl or, independently, at each occurrence of R^{a} and R^{b} attached to the same carbon atom, R^{a} and R^{b}, together with the carbon to which they are attached, form a cycloalkyl;
R is, at each occurrence, independently H, halo, —CN, —OH, —NH₂, or C₁₋₆ alkyl
m is 0-5;
n is 0-5;
p is 0-5
q is 1-3;
u is, at each occurrence, independently 0-3;
v is, at each occurrence, independently 0-3;
w is 0-3;
x is 0-3;
y is 0-3; and
z is 0-3.

In one embodiment is provided a compound of Formula (III), or a pharmaceutically acceptable isomer, racemate, hydrate, solvate, isotope, or salt thereof, wherein Q¹, Q², and Q³ are each CR⁶. In another embodiment is provided a compound of Formula (III), or a pharmaceutically acceptable isomer, racemate, hydrate, solvate, isotope, or salt thereof, wherein Q¹ is N and Q² and Q³ are each CR⁶. In another embodiment is provided a compound of Formula (III), or a pharmaceutically acceptable isomer, racemate, hydrate, solvate, isotope, or salt thereof, wherein Q² is N and Q¹ and Q³ are each CR⁶. In another embodiment is provided a compound of Formula (III), or a pharmaceutically acceptable isomer, racemate, hydrate, solvate, isotope, or salt thereof, wherein Q³ is N and Q¹ and Q² are each CR⁶.

In one embodiment is provided a compound of any one of Formula (I), (II), or (III), or a pharmaceutically acceptable isomer, racemate, hydrate, solvate, isotope, or salt thereof, wherein w is 0.

In one embodiment is provided a compound of any one of Formula (I), (II), or (III), or a pharmaceutically acceptable isomer, racemate, hydrate, solvate, isotope, or salt thereof, wherein w is 1. In a further embodiment, R^{a} is H. In another embodiment, R^{a} is C₁₋₆ alkyl. In a further embodiment, R^{a} is methyl. In one embodiment, R^{b} is H. In one embodiment, R^{b} is C₁₋₆ alkyl. In a further embodiment, R^{b} is methyl. In one embodiment, R^{a} and R^{b}, together with the carbon atom to which they are attached, form a cycloalkyl. In one embodiment, R^{a} and R^{b}, together with the carbon atom to which they are attached, form cyclopropyl, cyclobutyl, or cyclopentyl.

In one embodiment is provided a compound of any one of Formula (I), (II), or (III), or a pharmaceutically acceptable isomer, racemate, hydrate, solvate, isotope, or salt thereof, wherein w is 2 or 3. In a further embodiment, at least one R^{a} is H. In another embodiment, at least one R^{a} is C₁₋₆ alkyl. In a further embodiment, at least one R^{a} is methyl. In one embodiment, at least one R^{b} is H. In one embodiment, at least one R^{b} is C₁₋₆ alkyl. In a further embodiment, at least one R^{b} is methyl. In one embodiment, at least one R^{a} and R^{b}, together with the carbon atom to which they are attached, form a cycloalkyl. In a further embodiment, at least one R^{a} and R^{b}, together with the carbon atom to which they are attached, form cyclopropyl, cyclobutyl, or cyclopentyl.

In one embodiment is provided a compound of any one of Formula (I), (II), or (III), or a pharmaceutically acceptable isomer, racemate, hydrate, solvate, isotope, or salt thereof, wherein L¹ is —(CR′R″)_{q}—. In one embodiment, q is 1. In another embodiment, q is 2. In one embodiment, each R′ is H. In another embodiment, at least one R′ is C₁₋₆ alkyl. In one embodiment, at least one R′ is methyl. In one embodiment, each R″ is H. In another embodiment, at least one R″ is C₁₋₆ alkyl. In one embodiment, at least one R″ is methyl. In another embodiment, at least one R″ is C₁₋₆ alkenyl. In one embodiment, at least one R″ is vinyl. In another embodiment, at least one occurrence of R′ and R″ attached to the same carbon atom form a cycloalkyl together with the carbon to which they are attached. In one embodiment, at least one occurrence of R′ and R″ attached to the same carbon atom form a cyclopropyl or a cyclobutyl together with the carbon to which they are attached.

In one embodiment is provided a compound of any one of Formula (I), (II), or (III), or a pharmaceutically acceptable isomer, racemate, hydrate, solvate, isotope, or salt thereof, wherein L¹ is —(CR′R″))ᵤ—CR′═CR′—(CR′R″)ᵥ—. In one embodiment, L¹ is —CR′═CR′—. In one embodiment, each R′ is H. In another embodiment, at least one R′ is C₁₋₆ alkyl. In one embodiment, at least one R′ is methyl.

In one embodiment is provided a compound of any one of Formula (I), (II), or (III), or a pharmaceutically acceptable isomer, racemate, hydrate, solvate, isotope, or salt thereof, wherein L¹ is —(CR′R″)ᵤ—C≡C—(CR′R″)ᵥ—. In one embodiment, L¹ is —C≡C—.

In one embodiment is provided a compound of any one of Formula (I), (II), or (III), or a pharmaceutically acceptable isomer, racemate, hydrate, solvate, isotope, or salt thereof, wherein L¹ is —(CR′R″))ᵤ—O—(CR′R″)ᵥ—. In one embodiment, u is 0. In another embodiment, u is 1. In one embodiment, v is 0. In another embodiment, v is 1. In one embodiment, L¹ is —O—.

In one embodiment is provided a compound of any one of Formula (I), (II), or (III), or a pharmaceutically acceptable isomer, racemate, hydrate, solvate, isotope, or salt thereof, wherein L¹ is —(CR′R″))ᵤ—NR′—(CR′R″)ᵥ—. In one embodiment, u is 0. In another embodiment, u is 1. In one embodiment, v is 0. In another embodiment, v is 1. In one embodiment, L¹ is —NR′—. In one embodiment, R′ is H. In another embodiment, R′ is C₁₋₆ alkyl. In one embodiment, R′ is methyl, ethyl, or propyl.

In one embodiment is provided a compound of any one of Formula (I), (II), or (III), or a pharmaceutically acceptable isomer, racemate, hydrate, solvate, isotope, or salt thereof, wherein L¹ is —(CR′R″))ᵤ—C(O)—(CR′R″)ᵥ—. In one embodiment, u is 0. In another embodiment, u is 1. In one embodiment, v is 0. In another embodiment, v is 1. In one embodiment, L¹ is —C(O)—.

In one embodiment is provided a compound having the structure of Formula (III-A) or Formula (III-B):

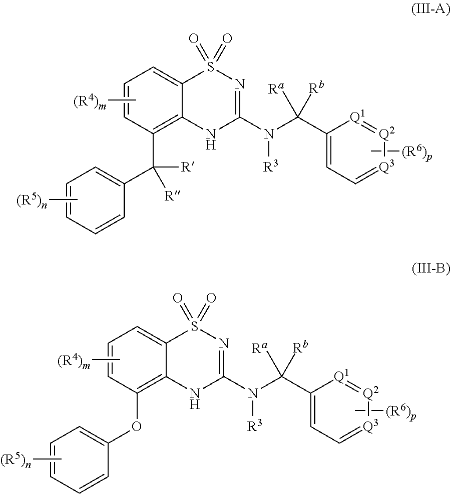

or a pharmaceutically acceptable isomer, racemate, hydrate, solvate, isotope, or salt thereof, wherein:
Q¹, Q², and Q³ are, at each occurrence, independently CR⁶ or N;
R³ is H or C₁₋₆ alkyl;
R⁴ is, at each occurrence, independently halo, —CN, —OR⁷, —N(R⁷)₂, C₁₋₆ alkyl, C₁₋₆ haloalkyl;
R⁵ is, at each occurrence, independently halo, —CN, —OR⁷, —N(R⁷)₂, —S(O)₂—R⁷, C₁₋₆ alkyl, C₁₋₆ haloalkyl, carbocycle, or heterocycle;
R⁶ is, at each occurrence, independently halo, —CN, —OR⁷, —N(R⁷)₂, —SR⁷, —C(O)OR⁷, —N(R⁸)C(O)R⁷, —N(R⁸)C(O)OR⁷, —S(O)₂—R⁷, —C₁₋₆ alkyl-R⁷, C₁₋₆ haloalkyl, carbocycle substituted with —(R⁷)ₓ, or heterocycle substituted with —(R⁷)ₓ;
R⁷ is, at each occurrence, independently H, —OR⁸, —N(R⁸)₂, —C(O)OR⁸, —NRC(O)OR⁸, —C₁₋₆ alkyl-R⁸, C₁₋₆ haloalkyl, carbocycle substituted with —(R⁸)_{y}, or heterocycle substituted with —(R⁸)_{y};
R⁸ is, at each occurrence, independently H, —OR, —NR₂, —C₁₋₆ alkyl-R, carbocycle substituted with —(R)_{z}, or heterocycle substituted with —(R)_{z};
R′ and R″ are, at each occurrence, independently H, halo, cyano, hydroxy, amino, C₁₋₆ alkyl, or C₁₋₆ alkenyl, or, independently, at each occurrence of R′ and R″ attached to the same carbon atom, R′ and R″, together with the carbon to which they are attached, form a cycloalkyl;
R^{a} and R^{b} are, at each occurrence, independently H, halo, cyano, hydroxy, amino, or C₁₋₆ alkyl or, independently, at each occurrence of R^{a} and R^{b} attached to the same carbon atom, R^{a} and R^{b}, together with the carbon to which they are attached, form a cycloalkyl;
R is, at each occurrence, independently H, halo, —CN, —OH, —NH₂, or C₁₋₆ alkyl
m is 0-5;
n is 0-5;
p is 0-5
x is 0-3;
y is 0-3; and
z is 0-3.

In one embodiment is provided a compound having the structure of Formula (III-A) or (III-B), or a pharmaceutically acceptable isomer, racemate, hydrate, solvate, isotope, or salt thereof, wherein Q¹, Q², and Q³ are each CR⁶. In another embodiment, Q¹ is N and Q² and Q³ are each CR⁶. In another embodiment, Q² is N and Q¹ and Q³ are each CR⁶. In another embodiment, Q³ is N and Q¹ and Q² are each CR⁶.

In one embodiment is provided a compound having the structure of Formula (III-A) or (III-B), or a pharmaceutically acceptable isomer, racemate, hydrate, solvate, isotope, or salt thereof, wherein R^{a} is H. In another embodiment, R^{a} is C₁₋₆ alkyl. In one embodiment, R^{a} is methyl.

In one embodiment is provided a compound having the structure of Formula (III-A) or (III-B), or a pharmaceutically acceptable isomer, racemate, hydrate, solvate, isotope, or salt thereof, wherein R^{b} is H. In another embodiment, R^{b} is C₁₋₆ alkyl. In one embodiment, R^{b} is methyl.

In one embodiment is provided a compound having the structure of Formula (III-A) or (III-B), or a pharmaceutically acceptable isomer, racemate, hydrate, solvate, isotope, or salt thereof, wherein R^{a} and R^{b}, together with the carbon atom to which they are attached, form a cycloalkyl. In one embodiment, R^{a} and R^{b}, together with the carbon atom to which they are attached, form cyclopropyl, cyclobutyl, or cyclopentyl.

In one embodiment is provided a compound having the structure of Formula (I), (II), (III), (III-A), or (III-B), or a pharmaceutically acceptable isomer, racemate, hydrate, solvate, isotope, or salt thereof, wherein m is 0. In another embodiment, m is 1 or 2. In another embodiment, m is 1. In another embodiment, m is 2.

In one embodiment is provided a compound having the structure of Formula (I), (II), (III), (III-A), or (III-B), or a pharmaceutically acceptable isomer, racemate, hydrate, solvate, isotope, or salt thereof, wherein R⁴ is C₁₋₆ alkyl. In one embodiment, at least one R⁴ is methyl.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁴ is halo. In one embodiment, at least one R⁴ is Cl or F.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁴ is —CN.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁴ is C₁₋₆ alkoxy. In one embodiment, at least one R⁴ is —OCH₃.

In one embodiment is provided a compound having the structure of Formula (I), (II), (III), (III-A), or (III-B), or a pharmaceutically acceptable isomer, racemate, hydrate, solvate, isotope, or salt thereof, wherein n is 0. In another embodiment, n is 1, 2, or 3. In one embodiment, n is 1. In one embodiment, n is 2. In one embodiment, n is 3.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁵ is C₁₋₆ alkyl. In one embodiment, at least one R⁵ is methyl, ethyl, propyl, or butyl. In one embodiment, at least one R⁵ is methyl. In one embodiment, at least one R⁵ is ethyl. In one embodiment, at least one R⁵ is propyl. In one embodiment, at least one R⁵ is butyl.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁵ is halo. In one embodiment, at least one R⁵ is Cl or F. In one embodiment, at least one R⁵ is Cl. In another embodiment, at least one R⁵ is F.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁵ is —CN.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁵ is C₁₋₆ alkoxy. In one embodiment, at least one R⁵ is methoxy.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁵ is carbocycle. In one embodiment, at least one R⁵ is phenyl.

In one embodiment is provided a compound having the structure of Formula (I), (II), (III), (III-A), or (III-B), or a pharmaceutically acceptable isomer, racemate, hydrate, solvate, isotope, or salt thereof, wherein p is 0. In another embodiment, p is 1, 2, or 3. In one embodiment, p is 1. In one embodiment, p is 2. In one embodiment, p is 3.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is halo. In one embodiment, at least one R⁶ is Cl or F. In one embodiment, at least one R⁶ is Cl. In another embodiment, at least one R⁶ is F.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is —CN.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is —OR⁷. In one embodiment, at least one R⁷ is H, —C₁₋₆ alkyl-R⁸, or C₁₋₆ haloalkyl. In one embodiment, at least one R⁷ is H. In one embodiment, at least one R⁷ is —C₁₋₆ alkyl-R⁸. In one embodiment, at least one R⁸ is H, —C₁₋₆ alkyl-R⁸, or carbocycle. In one embodiment, at least one R⁷ is C₁₋₆ haloalkyl. In one embodiment, at least one R⁶ is:

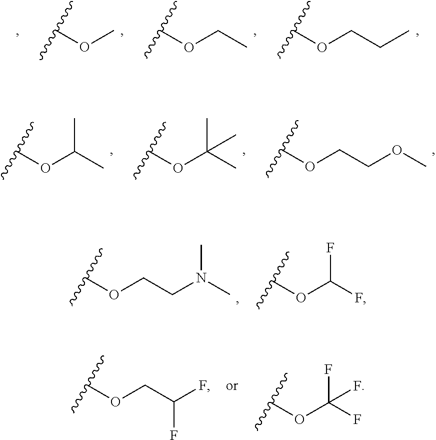

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is —N(R⁷)₂. In one embodiment, at least one R⁷ is H or —C₁₋₆ alkyl-R⁸. In one embodiment, at least one R⁸ is H, —C₁₋₆ alkyl-R⁸, or carbocycle. In one embodiment, at least one R⁶ is:

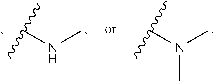

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is —SR⁷ or —S(O)₂R⁷. In one embodiment, at least one R⁷ is —N(R⁸)₂, —C₁₋₆ alkyl-R⁸, or carbocycle substituted with —(R⁸)_{y}. In one embodiment, at least one R⁸ is H, —C₁₋₆ alkyl-R⁸, or carbocycle. In one embodiment, at least one R⁶ is:

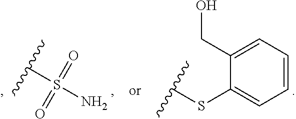 [SMILES: *S(N)(=O)=O ; *Sc1ccccc1CO]

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is —C(O)OR⁷, —N(R⁸)C(O)R⁷, —N(R⁸)C(O)OR⁷, or —C(O)N(R⁸)₂. In one embodiment, at least one R⁷ is H or —C₁₋₆ alkyl-R⁸. In one embodiment, at least one R⁸ is H, —C₁₋₆ alkyl-R⁸, or carbocycle. In one embodiment, at least one R⁸ is H, —C₁₋₆ alkyl-R⁸, or carbocycle. In one embodiment, at least one R⁶ is:

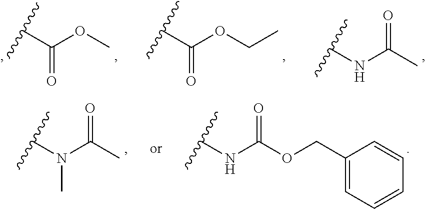

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is —C₁₋₆ alkyl-R⁷. In one embodiment, R⁷ is H. In one embodiment, at least one R⁶ is methyl, ethyl, propyl, or butyl. In another embodiment, R⁷ is —OR⁸, —N(R⁸)₂, —C(O)OR⁸, —NHC(O)OR⁸, or heterocycle substituted with —(R⁸)_{y}. In one embodiment, at least one R⁸ is H, —C₁₋₆ alkyl-R⁸, or carbocycle. In one embodiment, at least one R⁶ is:

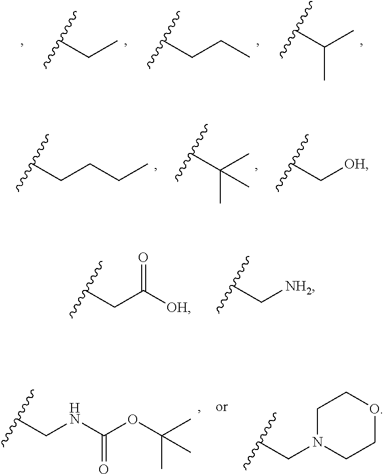

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is —C₁₋₆ haloalkyl. In one embodiment, at least one R⁶ is —CHF₂ or —CF₃.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is carbocycle substituted with —(R⁷)ₓ. In one embodiment, at least one R⁶ is phenyl substituted with —(R⁷)ₓ. In one embodiment, x is 0. In another embodiment, x is 1, 2, or 3. In one embodiment, x is 1. In one embodiment, x is 2. In one embodiment, x is 3.

In one embodiment is provided a compound having the structure of Formula (I), (II), (III), (III-A), or (III-B), or a pharmaceutically acceptable isomer, racemate, hydrate, solvate, isotope, or salt thereof, wherein at least one R⁶ is heterocycle substituted with —(R⁷)ₓ. In one embodiment, at least one R⁶ is pyrazolyl, thiophenyl, or tetrazolyl substituted with —(R⁷)ₓ. In one embodiment, x is 0. In another embodiment, x is 1, 2, or 3. In one embodiment, x is 1. In one embodiment, x is 2. In one embodiment, x is 3. In one embodiment, at least one R⁶ is pyrazolyl, thiophenyl, or tetrazolyl.

In one embodiment is provided a compound having the structure of Formula (I), (II), (III), (III-A), or (III-B), or a pharmaceutically acceptable isomer, racemate, hydrate, solvate, isotope, or salt thereof, wherein R³ and one R⁶, together with the atoms to which they are attached, form a ring. In one embodiment, R³ and one R⁶, together with the atoms to which they are attached, form a 6-membered ring fused with R².

In one embodiment is provided a compound having the structure of Formula (III-C), or Formula (III-D), Formula (III-E), or Formula (III-F):

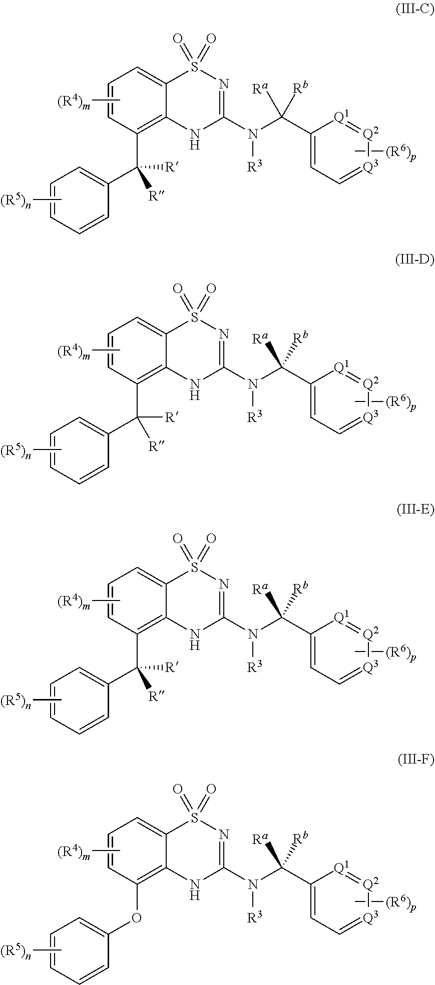

or a pharmaceutically acceptable isomer, racemate, hydrate, solvate, isotope, or salt thereof, wherein R′, R″, R^{a}, R^{b}, R³, R⁴, R⁵, R⁶, Q¹, Q², Q³, n, m, and p are as defined above in the context of Formulas (I), (II), (III), (III-A), or (III-B).

In another embodiments of Formulas (III-C), (III-D), (III-E) and (III-F), R′ is H.

In another embodiments of Formulas (III-C), (III-D), (III-E) and (III-F), R″ is H.

In another embodiments of Formulas (III-C), (III-D), (III-E) and (III-F), R′ is alkyl.

In another embodiments of Formulas (III-C), (III-D), (III-E) and (III-F), R′ is methyl.

In another embodiments of Formulas (III-C), (III-D), (III-E) and (III-F), R″ is alkyl.

In another embodiments of Formulas (III-C), (III-D), (III-E) and (III-F), R″ is methyl.

In another embodiments of Formulas (III-C), (III-D), (III-E) and (III-F), R′ and R″ are H.

In another embodiments of Formulas (III-C), (III-D), (III-E) and (III-F), R′ is alkyl and R″ is H.

In another embodiments of Formulas (III-C), (III-D), (III-E) and (III-F), R′ is methyl and R″ is H.

In another embodiments of Formulas (III-C), (III-D), (III-E) and (III-F), R′ is H and R″ is alky.

In another embodiments of Formulas (III-C), (III-D), (III-E) and (III-F), R′ is H and R″ is methyl.

In another embodiments of Formulas (III-C), (III-D), (III-E) and (III-F), R^{a} is H.

In another embodiments of Formulas (III-C), (III-D), (III-E) and (III-F), R^{b} is H.

In another embodiments of Formulas (III-C), (III-D), (III-E) and (III-F), R^{a} is alkyl.

In another embodiments of Formulas (III-C), (III-D), (III-E) and (III-F), R^{a} is methyl.

In another embodiments of Formulas (III-C), (III-D), (III-E) and (III-F), R^{b} is alkyl.

In another embodiments of Formulas (III-C), (III-D), (III-E) and (III-F), R^{b} is methyl.

In another embodiments of Formulas (III-C), (III-D), (III-E) and (III-F), R^{a} and R^{b} are H.

In another embodiments of Formulas (III-C), (III-D), (III-E) and (III-F), R^{a} is alky and R^{b} is H.

In another embodiments of Formulas (III-C), (III-D), (III-E) and (III-F), R^{a} is methyl and R^{b} is H.

In another embodiments of Formulas (III-C), (III-D), (III-E) and (III-F), R^{a} is H and R^{b} is alkyl.

In another embodiments of Formulas (III-C), (III-D), (III-E) and (III-F), R^{a} is H and R^{b} is methyl.

In another embodiments of Formulas (III-C), (III-D), (III-E) and (III-F), R′ is H, R″ is H, R^{a} is H and R^{b} is H.

In another embodiments of Formulas (III-C), (III-D), (III-E) and (III-F), R′ is alkyl, R″ is H, R^{a} is H and R^{b} is H.

In another embodiments of Formulas (II-C), (III-D), (III-E) and (III-F), R′ is methyl, R″ is H, R^{a} is H and R^{b} is H.

In another embodiments of Formulas (III-C), (III-D), (III-E) and (III-F), R′ is H, R″ is alkyl, R^{a} is H and R^{b} is H.

In another embodiments of Formulas (III-C), (III-D), (III-E) and (III-F), R′ is H, R″ is methyl, R^{a} is H and R^{b} is H.

In another embodiments of Formulas (III-C), (III-D), (III-E) and (III-F), R′ is H, R″ is H, R^{a} is alkyl and R^{b} is H.

In another embodiments of Formulas (III-C), (III-D), (III-E) and (III-F), R′ is H, R″ is H, R^{a} is methyl and R^{b} is H.

In another embodiments of Formulas (III-C), (III-D), (III-E) and (III-F), R′ is H, R″ is H, R^{a} is H and R^{b} is alkyl.

In another embodiments of Formulas (III-C), (III-D), (III-E) and (III-F), R′ is H, R″ is H, R^{a} is H and R^{b} is methyl.

Representative compounds of Formula (I), and Formulas (II), (III), (III-A), (III-B), (III-C), (III-D), (III-E) and (III-F) as applicable, include the compounds having the structure of those listed in Table 1 and Examples 1-20, below, as well as pharmaceutically acceptable isomers, racemates, tautomers, hydrates, solvates, isotopes, or salts thereof. To this end, representative compounds are identified herein by their respective “Compound Number”, which is sometimes abbreviated as “Compound No.”, “Cmpd. No.”, “Cpd. No.”, or “No.”

TABLE 1

| Cpd | | |
|---|---|---|
| No. | Structure | Name |
| 1-1 | 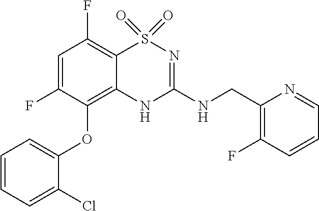 [SMILES: O=S1(=O)N=C(NCc2ncccc2F)Nc2c(Oc3ccccc3Cl)c(F)cc(F)c21] | 5-(2-chlorophenoxy)-6,8-difluoro- 3-(((3-fluoropyridin-2- yl)methyl)amino)-4H- benzo[e][1,2,4]thiadiazine 1,1- dioxide |
| 1-2 | 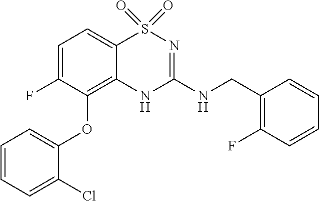 [SMILES: O=S1(=O)N=C(NCc2ccccc2F)Nc2c1ccc(F)c2Oc1ccccc1Cl] | 5-(2-chlorophenoxy)-6-fluoro-3- ((2-fluorobenzyl)amino)-4H- benzo[e][1,2,4]thiadiazine 1,1- dioxide |
| 1-3 | 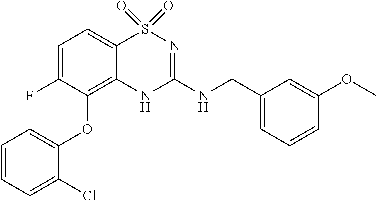 [SMILES: COc1cccc(CNC2=NS(=O)(=O)c3ccc(F)c(Oc4ccccc4Cl)c3N2)c1] | 5-(2-chlorophenoxy)-6-fluoro-3- ((3-methoxybenzyl)amino)-4H- benzo[e][1,2,4]thiadiazine 1,1- dioxide |
| 1-4 | 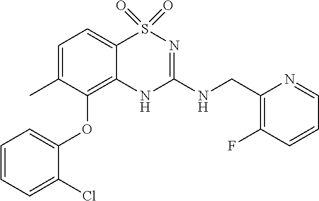 [SMILES: Cc1ccc2c(c1Oc1ccccc1Cl)NC(NCc1ncccc1F)=NS2(=O)=O] | 5-(2-chlorophenoxy)-3-(((3- fluoropyridin-2-yl)methyl)amino)- 6-methyl-4H- benzo[e][1,2,4]thiadiazine 1,1- dioxide |
| 1-5 | 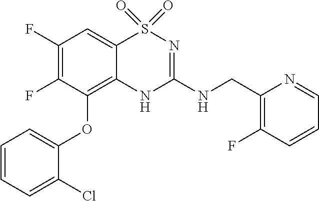 [SMILES: O=S1(=O)N=C(NCc2ncccc2F)Nc2c1cc(F)c(F)c2Oc1ccccc1Cl] | 5-(2-chlorophenoxy)-6,7-difluoro- 3-(((3-fluoropyridin-2- yl)methyl)amino)-4H- benzo[e][1,2,4]thiadiazine 1,1- dioxide |
| 1-6 | 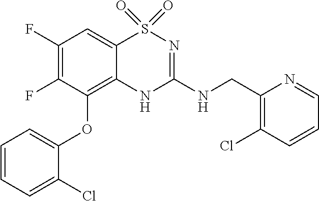 [SMILES: O=S1(=O)N=C(NCc2ncccc2Cl)Nc2c1cc(F)c(F)c2Oc1ccccc1Cl] | 5-(2-chlorophenoxy)-3-(((3- chloropyridin-2-yl)methyl)amino)- 6,7-difluoro-4H- benzo[e][1,2,4]thiadiazine 1,1- dioxide |
| 1-7 | 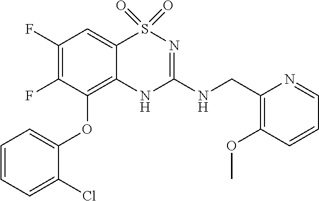 [SMILES: COc1cccnc1CNC1=NS(=O)(=O)c2cc(F)c(F)c(Oc3ccccc3Cl)c2N1] | 5-(2-chlorophenoxy)-6,7-difluoro- 3-(((3-methoxypyridin-2- yl)methyl)amino)-4H- benzo[e][1,2,4]thiadiazine 1,1- dioxide |
| 1-8 | 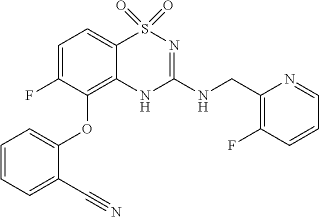 [SMILES: N#Cc1ccccc1Oc1c(F)ccc2c1NC(NCc1ncccc1F)=NS2(=O)=O] | 2-((6-fluoro-3-(((3-fluoropyridin- 2-yl)methyl)amino)-1,1-dioxido- 4H-benzo[e][1,2,4]thiadiazin-5- yl)oxy)benzonitrile |
| 1-9 | 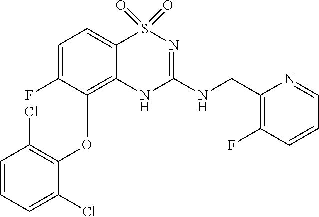 [SMILES: O=S1(=O)N=C(NCc2ncccc2F)Nc2c1ccc(F)c2Oc1c(Cl)cccc1Cl] | 5-(2,6-dichlorophenoxy)-6-fluoro- 3-(((3-fluoropyridin-2- yl)methyl)amino)-4H- benzo[e][1,2,4]thiadiazine 1,1- dioxide |
| 1-10 | 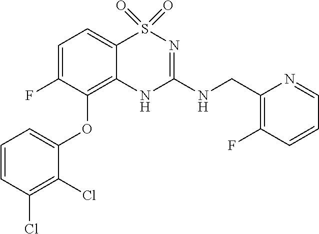 [SMILES: O=S1(=O)N=C(NCc2ncccc2F)Nc2c1ccc(F)c2Oc1cccc(Cl)c1Cl] | 5-(2,3-dichlorophenoxy)-6-fluoro- 3-(((3-fluoropyridin-2- yl)methyl)amino)-4H- benzo[e][1,2,4]thiadiazine 1,1- dioxide |
| 1-11 | 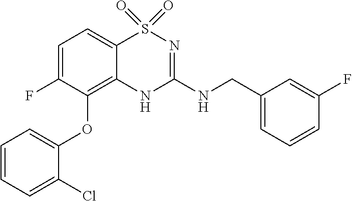 [SMILES: O=S1(=O)N=C(NCc2cccc(F)c2)Nc2c1ccc(F)c2Oc1ccccc1Cl] | 5-(2-chlorophenoxy)-6-fluoro-3- ((3-fluorobenzyl)amino)-4H- benzo[e][1,2,4]thiadiazine 1,1- dioxide |
| 1-12 | 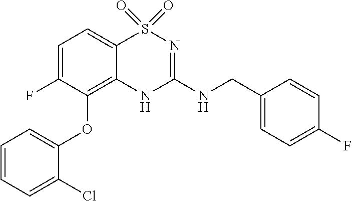 [SMILES: O=S1(=O)N=C(NCc2ccc(F)cc2)Nc2c1ccc(F)c2Oc1ccccc1Cl] | 5-(2-chlorophenoxy)-6-fluoro-3- ((4-fluorobenzyl)amino)-4H- benzo[e][1,2,4]thiadiazine 1,1- dioxide |
| 1-13 | 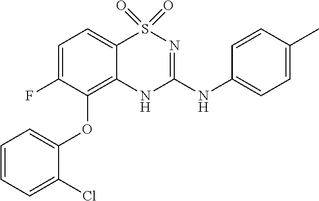 [SMILES: Cc1ccc(NC2=NS(=O)(=O)c3ccc(F)c(Oc4ccccc4Cl)c3N2)cc1] | 5-(2-chlorophenoxy)-6-fluoro-3- (p-tolylamino)-4H- benzo[e][1,2,4]thiadiazine 1,1- dioxide |
| 1-14 | 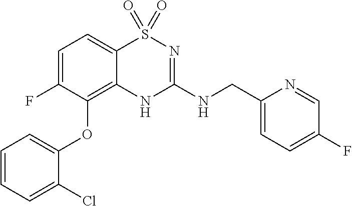 [SMILES: O=S1(=O)N=C(NCc2ccc(F)cn2)Nc2c1ccc(F)c2Oc1ccccc1Cl] | 5-(2-chlorophenoxy)-3-(((3,5- difluoropyridin-2- yl)methyl)amino)-6-fluoro-4H- benzo[e][1,2,4]thiadiazine 1,1- dioxide |
| 1-15 | 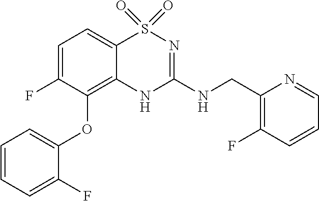 [SMILES: O=S1(=O)N=C(NCc2ncccc2F)Nc2c1ccc(F)c2Oc1ccccc1F] | 6-fluoro-5-(2-fluorophenoxy)-3- (((3-fluoropyridin-2- yl)methyl)amino)-4H- benzo[e][1,2,4]thiadiazine 1,1- dioxide |
| 1-16 | 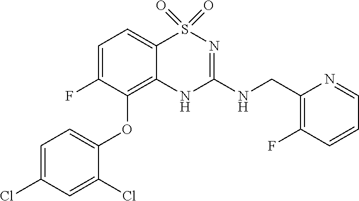 [SMILES: O=S1(=O)N=C(NCc2ncccc2F)Nc2c1ccc(F)c2Oc1ccc(Cl)cc1Cl] | 5-(2,4-dichlorophenoxy)-6-fluoro- 3-(((3-fluoropyridin-2- yl)methyl)amino)-4H- benzo[e][1,2,4]thiadiazine 1,1- dioxide |
| 1-17 | 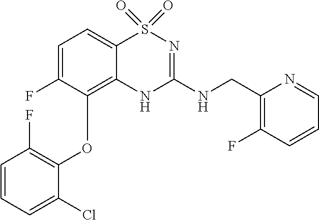 [SMILES: O=S1(=O)N=C(NCc2ncccc2F)Nc2c1ccc(F)c2Oc1c(F)cccc1Cl] | 5-(2-chloro-6-fluorophenoxy)-6- fluoro-3-(((3-fluoropyridin-2- yl)methyl)amino)-4H- benzo[e][1,2,4]thiadiazine 1,1- dioxide |
| 1-18 | 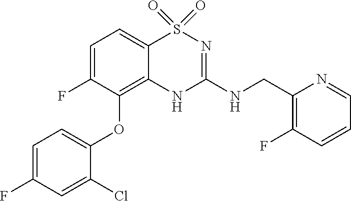 [SMILES: O=S1(=O)N=C(NCc2ncccc2F)Nc2c1ccc(F)c2Oc1ccc(F)cc1Cl] | 5-(2-chloro-4-fluorophenoxy)-6- fluoro-3-(((3-fluoropyridin-2- yl)methyl)amino)-4H- benzo[e][1,2,4]thiadiazine 1,1- dioxide |
| 1-19 | 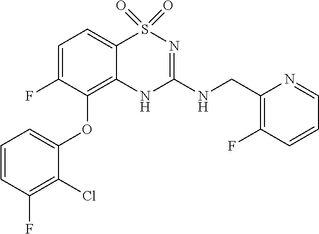 [SMILES: O=S1(=O)N=C(NCc2ncccc2F)Nc2c1ccc(F)c2Oc1cccc(F)c1Cl] | 5-(2-chloro-3-fluorophenoxy)-6- fluoro-3-(((3-fluoropyridin-2- yl)methyl)amino)-4H- benzo[e][1,2,4]thiadiazine 1,1- dioxide |
| 1-20 | 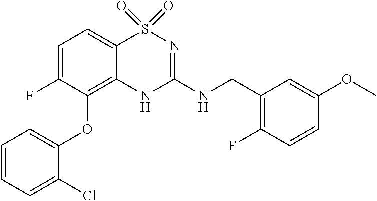 [SMILES: COc1ccc(F)c(CNC2=NS(=O)(=O)c3ccc(F)c(Oc4ccccc4Cl)c3N2)c1] | 5-(2-chlorophenoxy)-6-fluoro-3- ((2-fluoro-5- methoxybenzyl)amino)-4H- benzo[e][1,2,4]thiadiazine 1,1- dioxide |
| 1-21 | 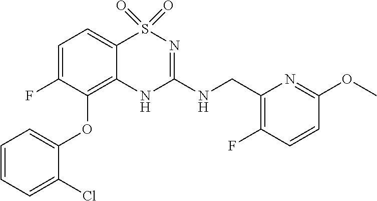 [SMILES: COc1ccc(F)c(CNC2=NS(=O)(=O)c3ccc(F)c(Oc4ccccc4Cl)c3N2)n1] | 5-(2-chlorophenoxy)-6-fluoro-3- (((3-fluoro-6-methoxypyridin-2- yl)methyl)amino)-4H- benzo[e][1,2,4]thiadiazine 1,1- dioxide |
| 1-22 | 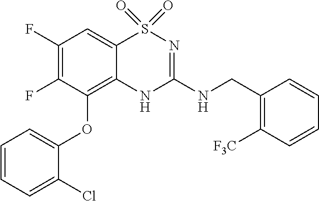 [SMILES: O=S1(=O)N=C(NCc2ccccc2C(F)(F)F)Nc2c1cc(F)c(F)c2Oc1ccccc1Cl] | 5-(2-chlorophenoxy)-6,7-difluoro- 3-((2- (trifluoromethyl)benzyl)amino)- 4H-benzo[e][1,2,4]thiadiazine 1,1- dioxide |
| 1-23 | 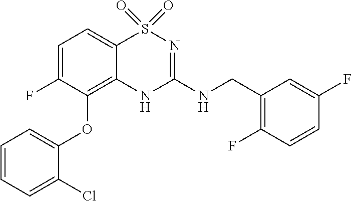 [SMILES: O=S1(=O)N=C(NCc2cc(F)ccc2F)Nc2c1ccc(F)c2Oc1ccccc1Cl] | 5-(2-chlorophenoxy)-3-((2,5- difluorobenzyl)amino)-6-fluoro- 4H-benzo[e][1,2,4]thiadiazine 1,1- dioxide |
| 1-24 | 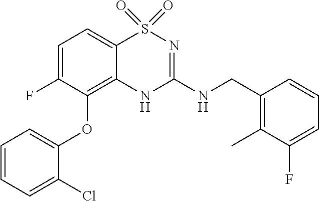 [SMILES: Cc1c(F)cccc1CNC1=NS(=O)(=O)c2ccc(F)c(Oc3ccccc3Cl)c2N1] | 5-(2-chlorophenoxy)-6-fluoro-3- ((3-fluoro-2-methylbenzyl)amino)- 4H-benzo[e][1,2,4]thiadiazine 1,1- dioxide |
| 1-25 | 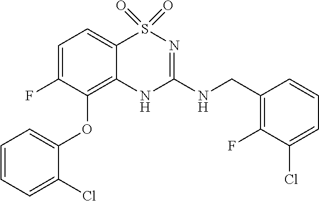 [SMILES: O=S1(=O)N=C(NCc2cccc(Cl)c2F)Nc2c1ccc(F)c2Oc1ccccc1Cl] | 3-((3-chloro-2- fluorobenzyl)amino)-5-(2- chlorophenoxy)-6-fluoro-4H- benzo[e][1,2,4]thiadiazine 1,1- dioxide |
| 1-26 | 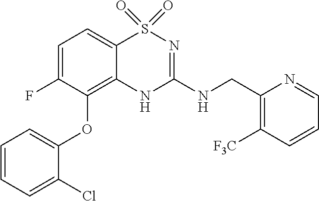 [SMILES: O=S1(=O)N=C(NCc2ncccc2C(F)(F)F)Nc2c1ccc(F)c2Oc1ccccc1Cl] | 5-(2-chlorophenoxy)-6-fluoro-3- (((3-(trifluoromethyl)pyridin-2- yl)methyl)amino)-4H- benzo[e][1,2,4]thiadiazine 1,1- dioxide |
| 1-27 | 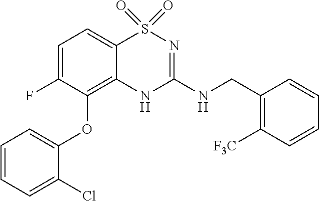 [SMILES: O=S1(=O)N=C(NCc2ccccc2C(F)(F)F)Nc2c1ccc(F)c2Oc1ccccc1Cl] | 5-(2-chlorophenoxy)-6-fluoro-3- ((2- (trifluoromethyl)benzyl)amino)- 4H-benzo[e][1,2,4]thiadiazine 1,1- dioxide |
| 1-28 | 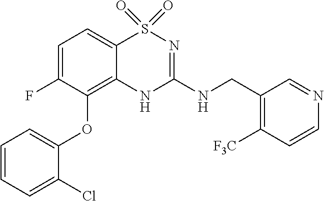 [SMILES: O=S1(=O)N=C(NCc2cnccc2C(F)(F)F)Nc2c1ccc(F)c2Oc1ccccc1Cl] | 5-(2-chlorophenoxy)-6-fluoro-3- (((4-(trifluoromethyl)pyridin-3- yl)methyl)amino)-4H- benzo[e][1,2,4]thiadiazine 1,1- dioxide |
| 1-29 | 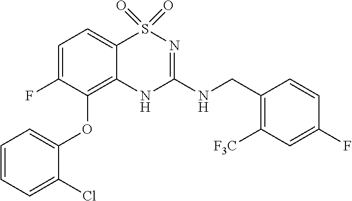 [SMILES: O=S1(=O)N=C(NCc2ccc(F)cc2C(F)(F)F)Nc2c1ccc(F)c2Oc1ccccc1Cl] | 5-(2-chlorophenoxy)-3-((2,4- difluorobenzyl)amino)-6-fluoro- 4H-benzo[e][1,2,4]thiadiazine 1,1- dioxide |
| 1-30 | 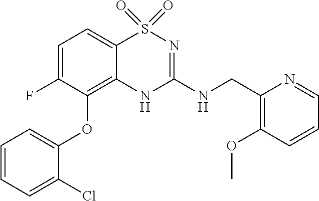 [SMILES: COc1cccnc1CNC1=NS(=O)(=O)c2ccc(F)c(Oc3ccccc3Cl)c2N1] | 5-(2-chlorophenoxy)-6-fluoro-3- (((3-methoxypyridin-2- yl)methyl)amino)-4H- benzo[e][1,2,4]thiadiazine 1,1- dioxide |
| 1-31 | 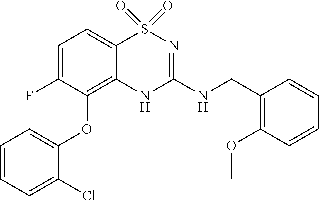 [SMILES: COc1ccccc1CNC1=NS(=O)(=O)c2ccc(F)c(Oc3ccccc3Cl)c2N1] | 5-(2-chlorophenoxy)-6-fluoro-3- ((2-methoxybenzyl)amino)-4H- benzo[e][1,2,4]thiadiazine 1,1- dioxide |
| 1-32 | 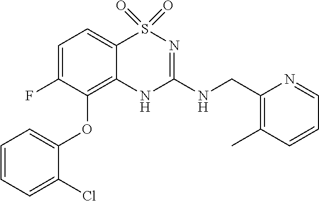 [SMILES: Cc1cccnc1CNC1=NS(=O)(=O)c2ccc(F)c(Oc3ccccc3Cl)c2N1] | 5-(2-chlorophenoxy)-6-fluoro-3- (((3-methylpyridin-2- yl)methyl)amino)-4H- benzo[e][1,2,4]thiadiazine 1,1- dioxide |
| 1-33 | 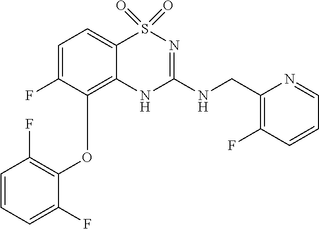 [SMILES: O=S1(=O)N=C(NCc2ncccc2F)Nc2c1ccc(F)c2Oc1c(F)cccc1F] | 5-(2,6-difluorophenoxy)-6-fluoro- 3-(((3-fluoropyridin-2- yl)methyl)amino)-4H- benzo[e][1,2,4]thiadiazine 1,1- dioxide |
| 1-34 | 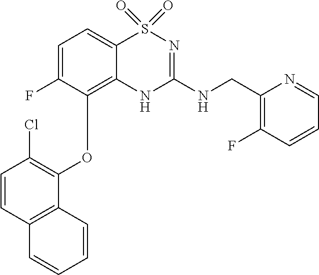 [SMILES: O=S1(=O)N=C(NCc2ncccc2F)Nc2c1ccc(F)c2Oc1c(Cl)ccc2ccccc12] | 5-((2-chloronaphthalen-1-yl)oxy)- 6-fluoro-3-(((3-fluoropyridin-2- yl)methyl)amino)-4H- benzo[e][1,2,4]thiadiazine 1,1- dioxide |
| 1-35 | 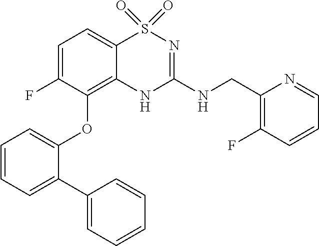 [SMILES: O=S1(=O)N=C(NCc2ncccc2F)Nc2c1ccc(F)c2Oc1ccccc1-c1ccccc1] | 5-([1,1′-biphenyl]-2-yloxy)-6- fluoro-3-(((3-fluoropyridin-2- yl)methyl)amino)-4H- benzo[e][1,2,4]thiadiazine 1,1- dioxide |
| 1-36 | 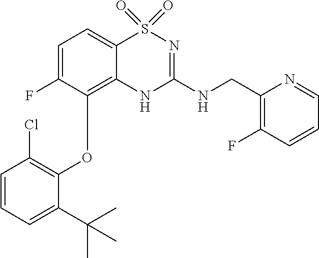 [SMILES: CC(C)(C)c1cccc(Cl)c1Oc1c(F)ccc2c1NC(NCc1ncccc1F)=NS2(=O)=O] | 5-(2-(tert-butyl)-6-chlorophenoxy)- 6-fluoro-3-(((3-fluoropyridin-2- yl)methyl)amino)-4H- benzo[e][1,2,4]thiadiazine 1,1- dioxide |
| 1-37 | 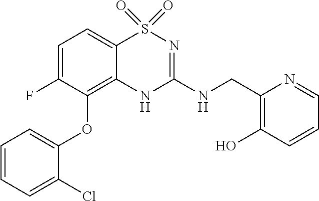 [SMILES: O=S1(=O)N=C(NCc2ncccc2O)Nc2c1ccc(F)c2Oc1ccccc1Cl] | 5-(2-chlorophenoxy)-6-fluoro-3- (((3-hydroxypyridin-2- yl)methyl)amino)-4H- benzo[e][1,2,4]thiadiazine 1,1- dioxide |
| 1-38 | 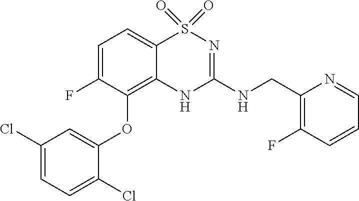 [SMILES: O=S1(=O)N=C(NCc2ncccc2F)Nc2c1ccc(F)c2Oc1cc(Cl)ccc1Cl] | 5-(2,5-dichlorophenoxy)-6-fluoro- 3-(((3-fluoropyridin-2- yl)methyl)amino)-4H- benzo[e][1,2,4]thiadiazine 1,1- dioxide |
| 1-39 | 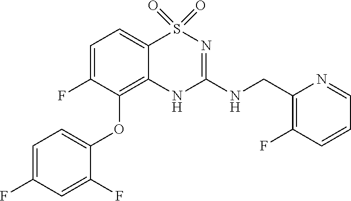 [SMILES: O=S1(=O)N=C(NCc2ncccc2F)Nc2c1ccc(F)c2Oc1ccc(F)cc1F] | 5-(2,4-difluorophenoxy)-6-fluoro- 3-(((3-fluoropyridin-2- yl)methyl)amino)-4H- benzo[e][1,2,4]thiadiazine 1,1- dioxide |
| 1-40 | 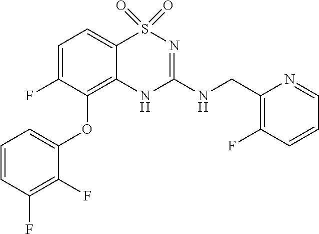 [SMILES: O=S1(=O)N=C(NCc2ncccc2F)Nc2c1ccc(F)c2Oc1cccc(F)c1F] | 5-(2,3-difluorophenoxy)-6-fluoro- 3-(((3-fluoropyridin-2- yl)methyl)amino)-4H- benzo[e][1,2,4]thiadiazine 1,1- dioxide |
| 1-41 | 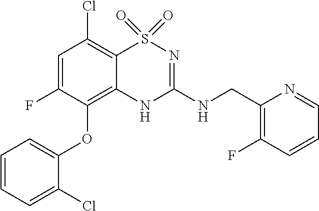 [SMILES: O=S1(=O)N=C(NCc2ncccc2F)Nc2c(Oc3ccccc3Cl)c(F)cc(Cl)c21] | 8-chloro-5-(2-chlorophenoxy)-6- fluoro-3-(((3-fluoropyridin-2- yl)methyl)amino)-4H- benzo[e][1,2,4]thiadiazine 1,1- dioxide |
| 1-42 | 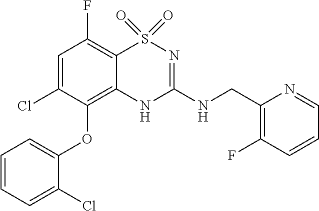 [SMILES: O=S1(=O)N=C(NCc2ncccc2F)Nc2c(Oc3ccccc3Cl)c(Cl)cc(F)c21] | 6-chloro-5-(2-chlorophenoxy)-8- fluoro-3-(((3-fluoropyridin-2- yl)methyl)amino)-4H- benzo[e][1,2,4]thiadiazine 1,1- dioxide |
| 1-43 | 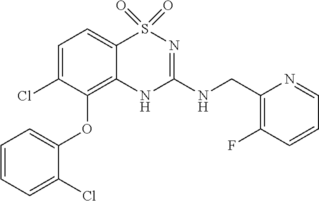 [SMILES: O=S1(=O)N=C(NCc2ncccc2F)Nc2c1ccc(Cl)c2Oc1ccccc1Cl] | 6-chloro-5-(2-chlorophenoxy)-3- (((3-fluoropyridin-2- yl)methyl)amino)-4H- benzo[e][1,2,4]thiadiazine 1,1- dioxide |
| 1-44 | 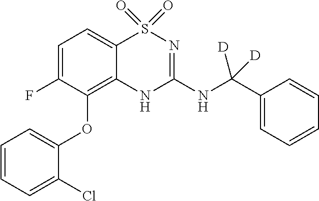 [SMILES: [2H]C([2H])(NC1=NS(=O)(=O)c2ccc(F)c(Oc3ccccc3Cl)c2N1)c1ccccc1] | 5-(2-chlorophenoxy)-6-fluoro-3- ((phenylmethyl-d2)amino)-4H- benzo[e][1,2,4]thiadiazine 1,1- dioxide |
| 1-45 | 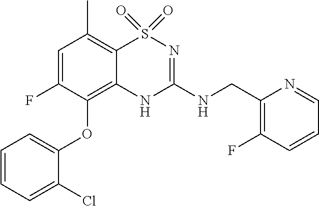 [SMILES: Cc1cc(F)c(Oc2ccccc2Cl)c2c1S(=O)(=O)N=C(NCc1ncccc1F)N2] | 5-(2-chlorophenoxy)-6-fluoro-3- (((3-fluoropyridin-2- yl)methyl)amino)-8-methyl-4H- benzo[e][1,2,4]thiadiazine 1,1- dioxide |
| 1-46 | 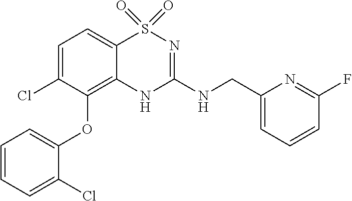 [SMILES: O=S1(=O)N=C(NCc2cccc(F)n2)Nc2c1ccc(Cl)c2Oc1ccccc1Cl] | 6-chloro-5-(2-chlorophenoxy)-3- (((6-fluoropyridin-2- yl)methyl)amino)-4H- benzo[e][1,2,4]thiadiazine 1,1- dioxide |
| 1-47 | 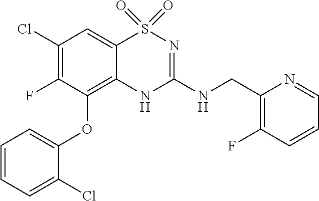 [SMILES: O=S1(=O)N=C(NCc2ncccc2F)Nc2c1cc(Cl)c(F)c2Oc1ccccc1Cl] | 7-chloro-5-(2-chlorophenoxy)-6- fluoro-3-(((3-fluoropyridin-2- yl)methyl)amino)-4H- benzo[e][1,2,4]thiadiazine 1,1- dioxide |
| 1-48 | 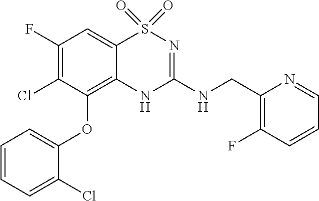 [SMILES: O=S1(=O)N=C(NCc2ncccc2F)Nc2c1cc(F)c(Cl)c2Oc1ccccc1Cl] | 6-chloro-5-(2-chlorophenoxy)-7- fluoro-3-(((3-fluoropyridin-2- yl)methyl)amino)-4H- benzo[e][1,2,4]thiadiazine 1,1- dioxide |
| 1-49 | 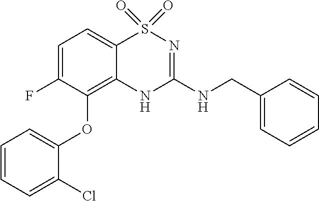 [SMILES: O=S1(=O)N=C(NCc2ccccc2)Nc2c1ccc(F)c2Oc1ccccc1Cl] | 3-(benzylamino)-5-(2- chlorophenoxy)-6-fluoro-4H- benzo[e][1,2,4]thiadiazine 1,1- dioxide |
| 1-50 | 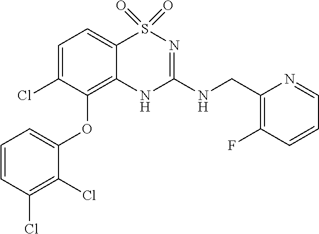 [SMILES: O=S1(=O)N=C(NCc2ncccc2F)Nc2c1ccc(Cl)c2Oc1cccc(Cl)c1Cl] | 6-chloro-5-(2,3-dichlorophenoxy)- 3-(((3-fluoropyridin-2- yl)methyl)amino)-4H- benzo[e][1,2,4]thiadiazine 1,1- dioxide |
| 1-51 | 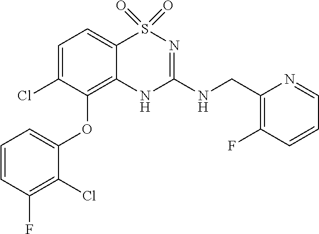 [SMILES: O=S1(=O)N=C(NCc2ncccc2F)Nc2c1ccc(Cl)c2Oc1cccc(F)c1Cl] | 6-chloro-5-(2-chloro-3- fluorophenoxy)-3-(((3- fluoropyridin-2-yl)methyl)amino)- 4H-benzo[e][1,2,4]thiadiazine 1,1- dioxide |
| 1-52 | 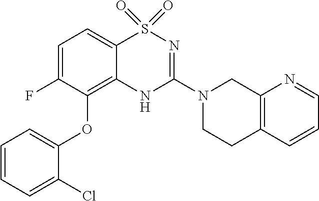 [SMILES: O=S1(=O)N=C(N2CCc3cccnc3C2)Nc2c1ccc(F)c2Oc1ccccc1Cl] | 5-(2-chlorophenoxy)-3-(5,8- dihydro-1,7-naphthyridin-7(6H)- yl)-6-fluoro-4H- benzo[e][1,2,4]thiadiazine 1,1- dioxide |
| 1-53 | 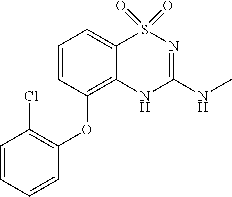 [SMILES: CNC1=NS(=O)(=O)c2cccc(Oc3ccccc3Cl)c2N1] | 5-(2-chlorophenoxy)-3- (methylamino)-4H- benzo[e][1,2,4]thiadiazine 1,1- dioxide |
| 1-54 | 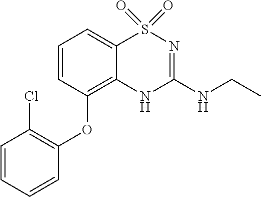 [SMILES: CCNC1=NS(=O)(=O)c2cccc(Oc3ccccc3Cl)c2N1] | 5-(2-chlorophenoxy)-3- (ethylamino)-4H- benzo[e][1,2,4]thiadiazine 1,1- dioxide |
| 1-55 | 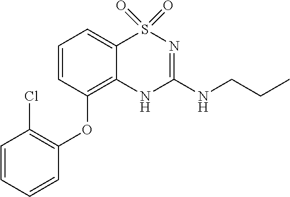 [SMILES: CCCNC1=NS(=O)(=O)c2cccc(Oc3ccccc3Cl)c2N1] | 5-(2-chlorophenoxy)-3- (propylamino)-4H- benzo[e][1,2,4]thiadiazine 1,1- dioxide |
| 1-56 | 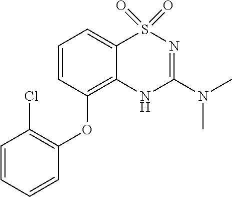 [SMILES: CN(C)C1=NS(=O)(=O)c2cccc(Oc3ccccc3Cl)c2N1] | 5-(2-chlorophenoxy)-3- (dimethylamino)-4H- benzo[e][1,2,4]thiadiazine 1,1- dioxide |
| 1-57 | 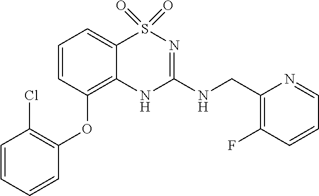 [SMILES: O=S1(=O)N=C(NCc2ncccc2F)Nc2c(Oc3ccccc3Cl)cccc21] | 5-(2-chlorophenoxy)-3-(((3- fluoropyridin-2-yl)methyl)amino)- 4H-benzo[e][1,2,4]thiadiazine 1,1- dioxide |
| 1-58 | 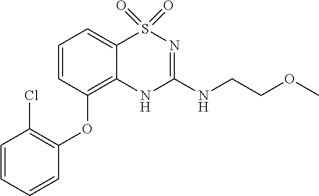 [SMILES: COCCNC1=NS(=O)(=O)c2cccc(Oc3ccccc3Cl)c2N1] | 5-(2-chlorophenoxy)-3-((2- methoxyethyl)amino)-4H- benzo[e][1,2,4]thiadiazine 1,1- dioxide |
| 1-59 | 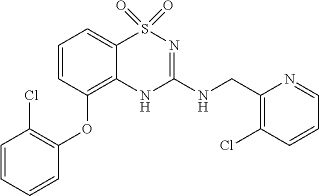 [SMILES: O=S1(=O)N=C(NCc2ncccc2Cl)Nc2c(Oc3ccccc3Cl)cccc21] | 5-(2-chlorophenoxy)-3-(((3- chloropyridin-2-yl)methyl)amino)- 4H-benzo[e][1,2,4]thiadiazine 1,1- dioxide |
| 1-60 | 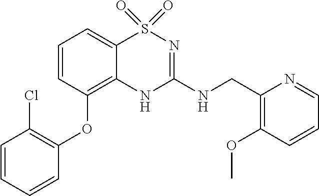 [SMILES: COc1cccnc1CNC1=NS(=O)(=O)c2cccc(Oc3ccccc3Cl)c2N1] | 5-(2-chlorophenoxy)-3-(((3- methoxypyridin-2- yl)methyl)amino)-4H- benzo[e][1,2,4]thiadiazine 1,1- dioxide |
| 1-61 | 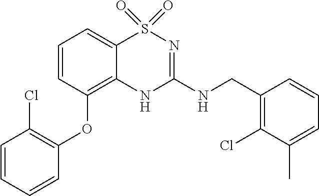 [SMILES: Cc1cccc(CNC2=NS(=O)(=O)c3cccc(Oc4ccccc4Cl)c3N2)c1Cl] | 3-((2-chloro-3- methylbenzyl)amino)-5-(2- chlorophenoxy)-4H- benzo[e][1,2,4]thiadiazine 1,1- dioxide |
| 1-62 | 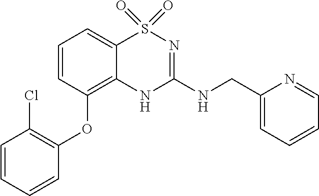 [SMILES: O=S1(=O)N=C(NCc2ccccn2)Nc2c(Oc3ccccc3Cl)cccc21] | 5-(2-chlorophenoxy)-3-((pyridin-2- ylmethyl)amino)-4H- benzo[e][1,2,4]thiadiazine 1,1- dioxide |
| 1-63 | 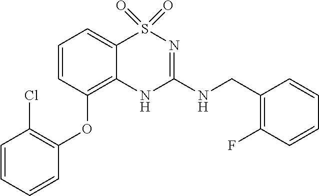 [SMILES: O=S1(=O)N=C(NCc2ccccc2F)Nc2c(Oc3ccccc3Cl)cccc21] | 5-(2-chlorophenoxy)-3-((2- fluorobenzyl)amino)-4H- benzo[e][1,2,4]thiadiazine 1,1- dioxide |
| 1-64 | 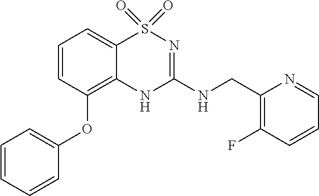 [SMILES: O=S1(=O)N=C(NCc2ncccc2F)Nc2c(Oc3ccccc3)cccc21] | 3-(((3-fluoropyridin-2- yl)methyl)amino)-5-phenoxy-4H- benzo[e][1,2,4]thiadiazine 1,1- dioxide |
| 1-65 | 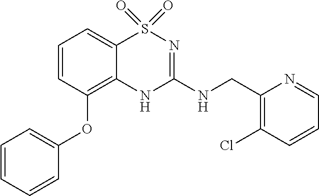 [SMILES: O=S1(=O)N=C(NCc2ncccc2Cl)Nc2c(Oc3ccccc3)cccc21] | 3-(((3-chloropyridin-2- yl)methyl)amino)-5-phenoxy-4H- benzo[e][1,2,4]thiadiazine 1,1- dioxide |
| 1-66 | 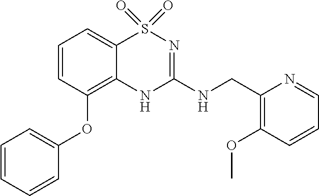 [SMILES: COc1cccnc1CNC1=NS(=O)(=O)c2cccc(Oc3ccccc3)c2N1] | 3-(((3-methoxypyridin-2- yl)methyl)amino)-5-phenoxy-4H- benzo[e][1,2,4]thiadiazine 1,1- dioxide |
| 1-67 | 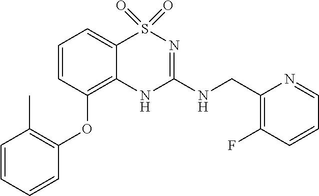 [SMILES: Cc1ccccc1Oc1cccc2c1NC(NCc1ncccc1F)=NS2(=O)=O] | 3-(((3-fluoropyridin-2- yl)methyl)amino)-5-(o-tolyloxy)- 4H-benzo[e][1,2,4]thiadiazine 1,1- dioxide |
| 1-68 | 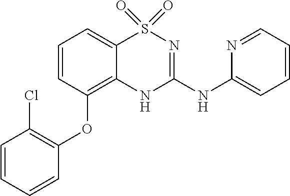 [SMILES: O=S1(=O)N=C(Nc2ccccn2)Nc2c(Oc3ccccc3Cl)cccc21] | 5-(2-chlorophenoxy)-3-(pyridin-2- ylamino)-4H- benzo[e][1,2,4]thiadiazine 1,1- dioxide |
| 1-69 | 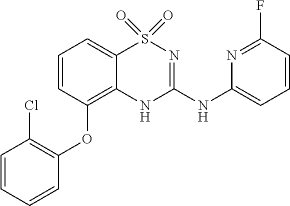 [SMILES: O=S1(=O)N=C(Nc2cccc(F)n2)Nc2c(Oc3ccccc3Cl)cccc21] | 5-(2-chlorophenoxy)-3-((6- fluoropyridin-2-yl)amino)-4H- benzo[e][1,2,4]thiadiazine 1,1- dioxide |
| 1-70 | 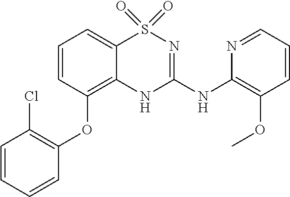 [SMILES: COc1cccnc1NC1=NS(=O)(=O)c2cccc(Oc3ccccc3Cl)c2N1] | 5-(2-chlorophenoxy)-3-((3- methoxypyridin-2-yl)amino)-4H- benzo[e][1,2,4]thiadiazine 1,1- dioxide |
| 1-71 | 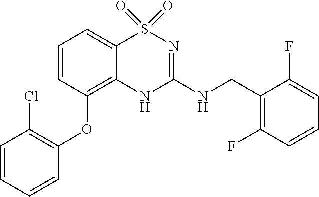 [SMILES: O=S1(=O)N=C(NCc2c(F)cccc2F)Nc2c(Oc3ccccc3Cl)cccc21] | 5-(2-chlorophenoxy)-3-((2,6- difluorobenzyl)amino)-4H- benzo[e][1,2,4]thiadiazine 1,1- dioxide |
| 1-72 | 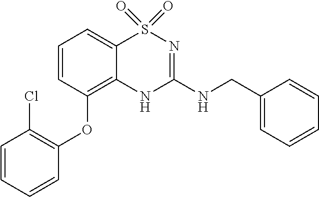 [SMILES: O=S1(=O)N=C(NCc2ccccc2)Nc2c(Oc3ccccc3Cl)cccc21] | 3-(benzylamino)-5-(2- chlorophenoxy)-4H- benzo[e][1,2,4]thiadiazine 1,1- dioxide |
| 1-73 | 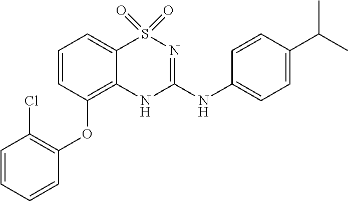 [SMILES: CC(C)c1ccc(NC2=NS(=O)(=O)c3cccc(Oc4ccccc4Cl)c3N2)cc1] | 5-(2-chlorophenoxy)-3-((4- isopropylphenyl)amino)-4H- benzo[e][1,2,4]thiadiazine 1,1- dioxide |
| 1-74 | 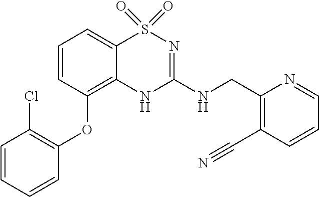 [SMILES: N#Cc1cccnc1CNC1=NS(=O)(=O)c2cccc(Oc3ccccc3Cl)c2N1] | 2-(((5-(2-chlorophenoxy)-1,1- dioxido-4H- benzo[e][1,2,4]thiadiazin-3- yl)amino)methyl)nicotinonitrile |
| 1-75 | 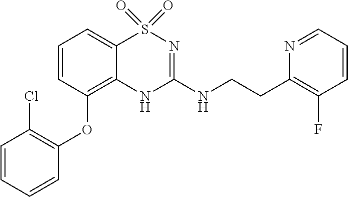 [SMILES: O=S1(=O)N=C(NCCc2ncccc2F)Nc2c(Oc3ccccc3Cl)cccc21] | 5-(2-chlorophenoxy)-3-((2-(3- fluoropyridin-2-yl)ethyl)amino)- 4H-benzo[e][1,2,4]thiadiazine 1,1- dioxide |
| 1-76 | 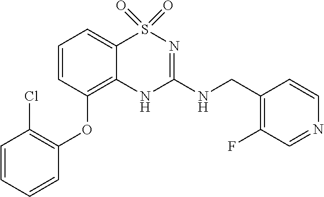 [SMILES: O=S1(=O)N=C(NCc2ccncc2F)Nc2c(Oc3ccccc3Cl)cccc21] | 5-(2-chlorophenoxy)-3-(((3- fluoropyridin-4-yl)methyl)amino)- 4H-benzo[e][1,2,4]thiadiazine 1,1- dioxide |
| 1-77 | 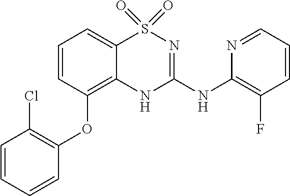 [SMILES: O=S1(=O)N=C(Nc2ncccc2F)Nc2c(Oc3ccccc3Cl)cccc21] | 5-(2-chlorophenoxy)-3-((3- fluoropyridin-2-yl)amino)-4H- benzo[e][1,2,4]thiadiazine 1,1- dioxide |
| 1-78 | 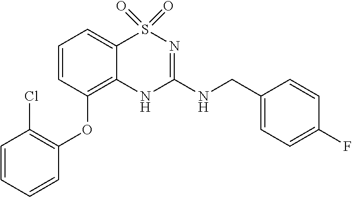 [SMILES: O=S1(=O)N=C(NCc2ccc(F)cc2)Nc2c(Oc3ccccc3Cl)cccc21] | 5-(2-chlorophenoxy)-3-((4- fluorobenzyl)amino)-4H- benzo[e][1,2,4]thiadiazine 1,1- dioxide |
| 1-79 | 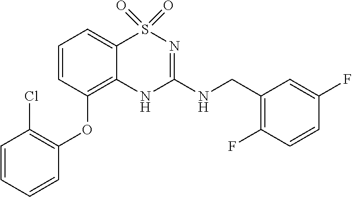 [SMILES: O=S1(=O)N=C(NCc2cc(F)ccc2F)Nc2c(Oc3ccccc3Cl)cccc21] | 5-(2-chlorophenoxy)-3-((2,5- difluorobenzyl)amino)-4H- benzo[e][1,2,4]thiadiazine 1,1- dioxide |
| 1-80 | 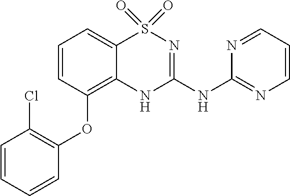 [SMILES: O=S1(=O)N=C(Nc2ncccn2)Nc2c(Oc3ccccc3Cl)cccc21] | 5-(2-chlorophenoxy)-3-(pyrimidin- 2-ylamino)-4H- benzo[e][1,2,4]thiadiazine 1,1- dioxide |
| 1-81 | 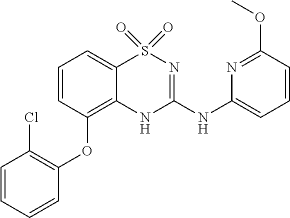 [SMILES: COc1cccc(NC2=NS(=O)(=O)c3cccc(Oc4ccccc4Cl)c3N2)n1] | 5-(2-chlorophenoxy)-3-((6- methoxypyridin-2-yl)amino)-4H- benzo[e][1,2,4]thiadiazine 1,1- dioxide |
| 1-82 | 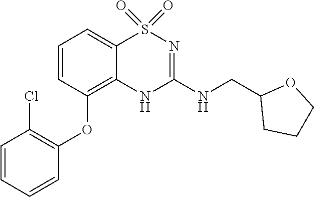 [SMILES: O=S1(=O)N=C(NCC2CCCO2)Nc2c(Oc3ccccc3Cl)cccc21] | 5-(2-chlorophenoxy)-3- (((tetrahydrofuran-2- yl)methyl)amino)-4H- benzo[e][1,2,4]thiadiazine 1,1- dioxide |
| 1-83 | 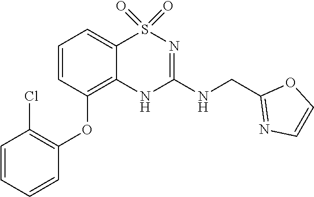 [SMILES: O=S1(=O)N=C(NCc2ncco2)Nc2c(Oc3ccccc3Cl)cccc21] | 5-(2-chlorophenoxy)-3-((oxazol-2- ylmethyl)amino)-4H- benzo[e][1,2,4]thiadiazine 1,1- dioxide |
| 1-84 | 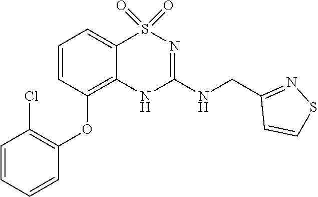 [SMILES: O=S1(=O)N=C(NCc2ccsn2)Nc2c(Oc3ccccc3Cl)cccc21] | 5-(2-chlorophenoxy)-3- ((isothiazol-3-ylmethyl)amino)- 4H-benzo[e][1,2,4]thiadiazine 1,1- dioxide |
| 1-85 | 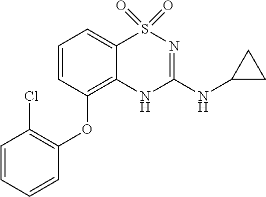 [SMILES: O=S1(=O)N=C(NC2CC2)Nc2c(Oc3ccccc3Cl)cccc21] | 5-(2-chlorophenoxy)-3- (cyclopropylamino)-4H- benzo[e][1,2,4]thiadiazine 1,1- dioxide |
| 1-86 | 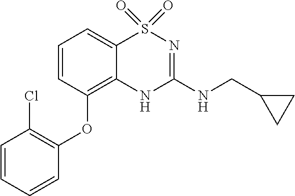 [SMILES: O=S1(=O)N=C(NCC2CC2)Nc2c(Oc3ccccc3Cl)cccc21] | 5-(2-chlorophenoxy)-3- ((cyclopropylmethyl)amino)-4H- benzo[e][1,2,4]thiadiazine 1,1- dioxide |
| 1-87 | 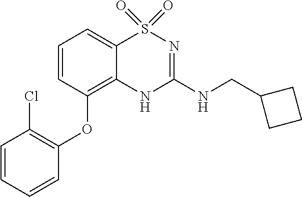 [SMILES: O=S1(=O)N=C(NCC2CCC2)Nc2c(Oc3ccccc3Cl)cccc21] | 5-(2-chlorophenoxy)-3- ((cyclobutylmethyl)amino)-4H- benzo[e][1,2,4]thiadiazine 1,1- dioxide |
| 1-88 | 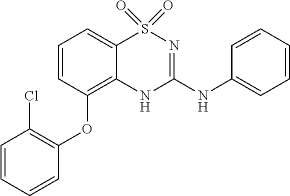 [SMILES: O=S1(=O)N=C(Nc2ccccc2)Nc2c(Oc3ccccc3Cl)cccc21] | 5-(2-chlorophenoxy)-3- (phenylamino)-4H- benzo[e][1,2,4]thiadiazine 1,1- dioxide |
| 1-89 | 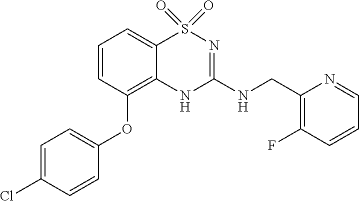 [SMILES: O=S1(=O)N=C(NCc2ncccc2F)Nc2c(Oc3ccc(Cl)cc3)cccc21] | 5-(4-chlorophenoxy)-3-(((3- fluoropyridin-2-yl)methyl)amino)- 4H-benzo[e][1,2,4]thiadiazine 1,1- dioxide |
| 1-90 | 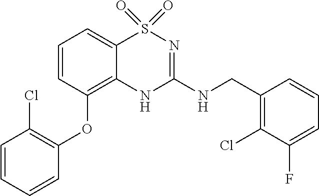 [SMILES: O=S1(=O)N=C(NCc2cccc(F)c2Cl)Nc2c(Oc3ccccc3Cl)cccc21] | 3-((2-chloro-3- fluorobenzyl)amino)-5-(2- chlorophenoxy)-4H- benzo[e][1,2,4]thiadiazine 1,1- dioxide |
| 1-91 | 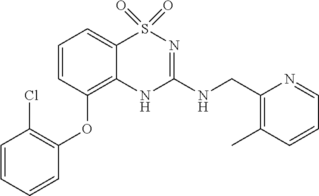 [SMILES: Cc1cccnc1CNC1=NS(=O)(=O)c2cccc(Oc3ccccc3Cl)c2N1] | 5-(2-chlorophenoxy)-3-(((3- methylpyridin-2-yl)methyl)amino)- 4H-benzo[e][1,2,4]thiadiazine 1,1- dioxide |
| 1-92 | 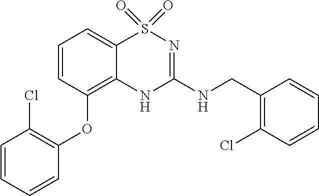 [SMILES: O=S1(=O)N=C(NCc2ccccc2Cl)Nc2c(Oc3ccccc3Cl)cccc21] | 3-((2-chlorobenzyl)amino)-5-(2- chlorophenoxy)-4H- benzo[e][1,2,4]thiadiazine 1,1- dioxide |
| 1-93 | 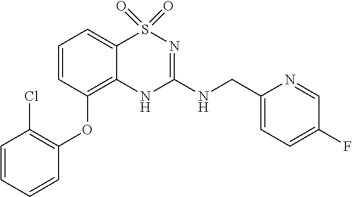 [SMILES: O=S1(=O)N=C(NCc2ccc(F)cn2)Nc2c(Oc3ccccc3Cl)cccc21] | 5-(2-chlorophenoxy)-3-(((5- fluoropyridin-2-yl)methyl)amino)- 4H-benzo[e][1,2,4]thiadiazine 1,1- dioxide |
| 1-94 | 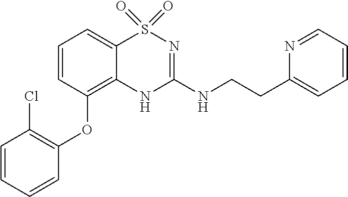 [SMILES: O=S1(=O)N=C(NCCc2ccccn2)Nc2c(Oc3ccccc3Cl)cccc21] | 5-(2-chlorophenoxy)-3-((2- (pyridin-2-yl)ethyl)amino)-4H- benzo[e][1,2,4]thiadiazine 1,1- dioxide |
| 1-95 | 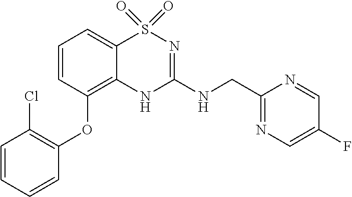 [SMILES: O=S1(=O)N=C(NCc2ncc(F)cn2)Nc2c(Oc3ccccc3Cl)cccc21] | 5-(2-chlorophenoxy)-3-(((5- fluoropyrimidin-2- yl)methyl)amino)-4H- benzo[e][1,2,4]thiadiazine 1,1- dioxide |
| 1-96 | 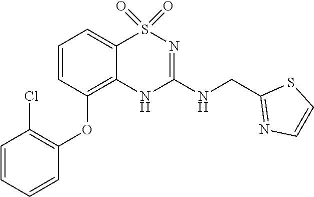 [SMILES: O=S1(=O)N=C(NCc2nccs2)Nc2c(Oc3ccccc3Cl)cccc21] | 5-(2-chlorophenoxy)-3-((thiazol-2- ylmethyl)amino)-4H- benzo[e][1,2,4]thiadiazine 1,1- dioxide |
| 1-97 | 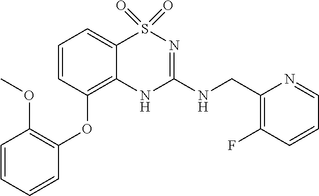 [SMILES: COc1ccccc1Oc1cccc2c1NC(NCc1ncccc1F)=NS2(=O)=O] | 3-(((3-fluoropyridin-2- yl)methyl)amino)-5-(2- methoxyphenoxy)-4H- benzo[e][1,2,4]thiadiazine 1,1- dioxide |
| 1-98 | 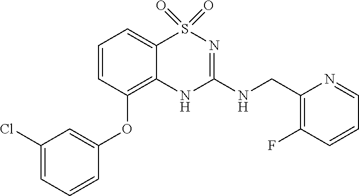 [SMILES: O=S1(=O)N=C(NCc2ncccc2F)Nc2c(Oc3cccc(Cl)c3)cccc21] | 5-(3-chlorophenoxy)-3-(((3- fluoropyridin-2-yl)methyl)amino)- 4H-benzo[e][1,2,4]thiadiazine 1,1- dioxide |
| 1-99 | 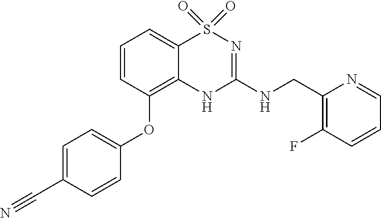 [SMILES: N#Cc1ccc(Oc2cccc3c2NC(NCc2ncccc2F)=NS3(=O)=O)cc1] | 4-((3-(((3-fluoropyridin-2- yl)methyl)amino)-1,1-dioxido-4H- benzo[e][1,2,4]thiadiazin-5- yl)oxy)benzonitrile |
| 1-100 | 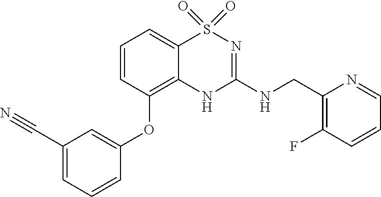 [SMILES: N#Cc1cccc(Oc2cccc3c2NC(NCc2ncccc2F)=NS3(=O)=O)c1] | 3-((3-(((3-fluoropyridin-2- yl)methyl)amino)-1,1-dioxido-4H- benzo[e][1,2,4]thiadiazin-5- yl)oxy)benzonitrile |
| 1-101 | 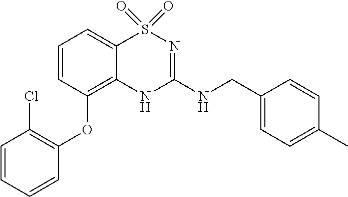 [SMILES: Cc1ccc(CNC2=NS(=O)(=O)c3cccc(Oc4ccccc4Cl)c3N2)cc1] | 5-(2-chlorophenoxy)-3-((4- methylbenzyl)amino)-4H- benzo[e][1,2,4]thiadiazine 1,1- dioxide |
| 1-102 | 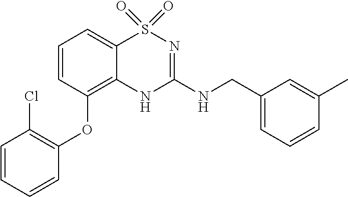 [SMILES: Cc1cccc(CNC2=NS(=O)(=O)c3cccc(Oc4ccccc4Cl)c3N2)c1] | 5-(2-chlorophenoxy)-3-((3- methylbenzyl)amino)-4H- benzo[e][1,2,4]thiadiazine 1,1- dioxide |
| 1-103 | 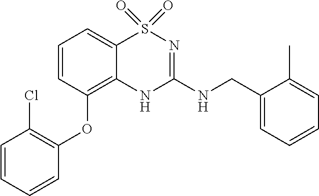 [SMILES: Cc1ccccc1CNC1=NS(=O)(=O)c2cccc(Oc3ccccc3Cl)c2N1] | 5-(2-chlorophenoxy)-3-((2- methylbenzyl)amino)-4H- benzo[e][1,2,4]thiadiazine 1,1- dioxide |
| 1-104 | 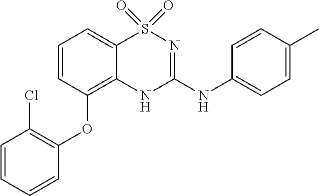 [SMILES: Cc1ccc(NC2=NS(=O)(=O)c3cccc(Oc4ccccc4Cl)c3N2)cc1] | 5-(2-chlorophenoxy)-3-(p- tolylamino)-4H- benzo[e][1,2,4]thiadiazine 1,1- dioxide |
| 1-105 | 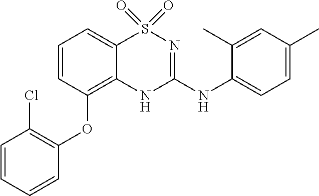 [SMILES: Cc1ccc(NC2=NS(=O)(=O)c3cccc(Oc4ccccc4Cl)c3N2)c(C)c1] | 5-(2-chlorophenoxy)-3-((2,4- dimethylphenyl)amino)-4H- benzo[e][1,2,4]thiadiazine 1,1- dioxide |
| 1-106 | 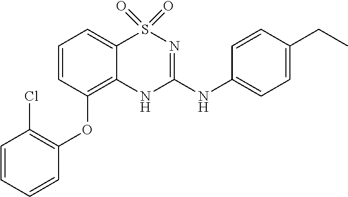 [SMILES: CCc1ccc(NC2=NS(=O)(=O)c3cccc(Oc4ccccc4Cl)c3N2)cc1] | 5-(2-chlorophenoxy)-3-((4- ethylphenyl)amino)-4H- benzo[e][1,2,4]thiadiazine 1,1- dioxide |
| 1-107 | 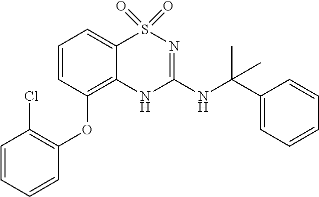 [SMILES: CC(C)(NC1=NS(=O)(=O)c2cccc(Oc3ccccc3Cl)c2N1)c1ccccc1] | 5-(2-chlorophenoxy)-3-((2- phenylpropan-2-yl)amino)-4H- benzo[e][1,2,4]thiadiazine 1,1- dioxide |
| 1-108 | 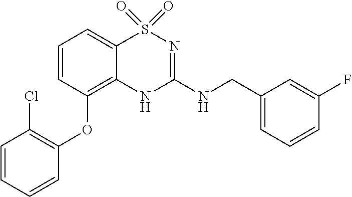 [SMILES: O=S1(=O)N=C(NCc2cccc(F)c2)Nc2c(Oc3ccccc3Cl)cccc21] | 5-(2-chlorophenoxy)-3-((3- fluorobenzyl)amino)-4H- benzo[e][1,2,4]thiadiazine 1,1- dioxide |
| 1-109 | 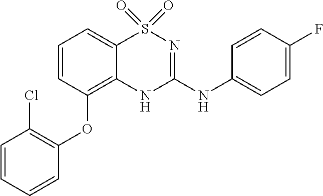 [SMILES: O=S1(=O)N=C(Nc2ccc(F)cc2)Nc2c(Oc3ccccc3Cl)cccc21] | 5-(2-chlorophenoxy)-3-((4- fluorophenyl)amino)-4H- benzo[e][1,2,4]thiadiazine 1,1- dioxide |
| 1-110 | 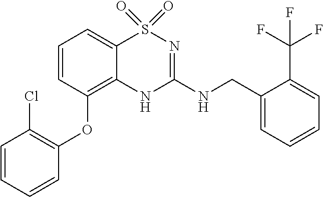 [SMILES: O=S1(=O)N=C(NCc2ccccc2C(F)(F)F)Nc2c(Oc3ccccc3Cl)cccc21] | 5-(2-chlorophenoxy)-3-((2- (trifluoromethyl)benzyl)amino)- 4H-benzo[e][1,2,4]thiadiazine 1,1- dioxide |
| 1-111 | 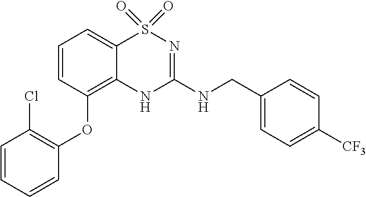 [SMILES: O=S1(=O)N=C(NCc2ccc(C(F)(F)F)cc2)Nc2c(Oc3ccccc3Cl)cccc21] | 5-(2-chlorophenoxy)-3-((4- (trifluoromethyl)benzyl)amino)- 4H-benzo[e][1,2,4]thiadiazine 1,1- dioxide |
| 1-112 | 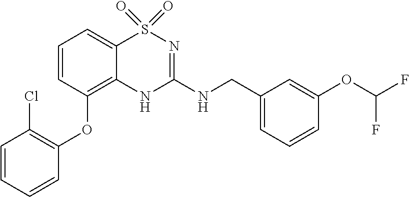 [SMILES: O=S1(=O)N=C(NCc2cccc(OC(F)F)c2)Nc2c(Oc3ccccc3Cl)cccc21] | 5-(2-chlorophenoxy)-3-((3- (difluoromethoxy)benzyl)amino)- 4H-benzo[e][1,2,4]thiadiazine 1,1- dioxide |
| 1-113 | 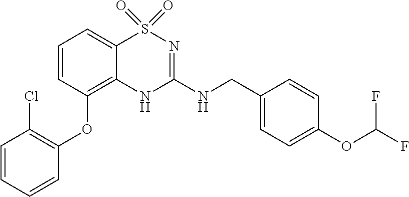 [SMILES: O=S1(=O)N=C(NCc2ccc(OC(F)F)cc2)Nc2c(Oc3ccccc3Cl)cccc21] | 5-(2-chlorophenoxy)-3-((4- (difluoromethoxy)benzyl)amino)- 4H-benzo[e][1,2,4]thiadiazine 1,1- dioxide |
| 1-114 | 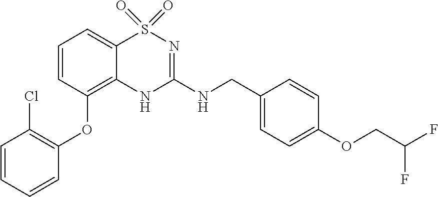 [SMILES: O=S1(=O)N=C(NCc2ccc(OCC(F)F)cc2)Nc2c(Oc3ccccc3Cl)cccc21] | 5-(2-chlorophenoxy)-3-((4-(2,2- difluoroethoxy)benzyl)amino)-4H- benzo[e][1,2,4]thiadiazine 1,1- dioxide |
| 1-115 | 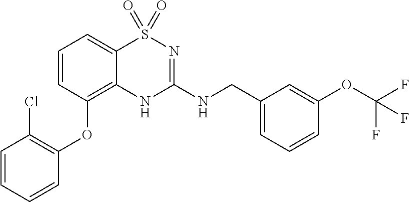 [SMILES: O=S1(=O)N=C(NCc2cccc(OC(F)(F)F)c2)Nc2c(Oc3ccccc3Cl)cccc21] | 5-(2-chlorophenoxy)-3-((3- (trifluoromethoxy)benzyl)amino)- 4H-benzo[e][1,2,4]thiadiazine 1,1- dioxide |
| 1-116 | 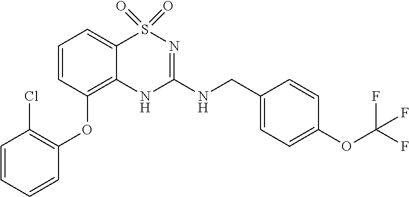 [SMILES: O=S1(=O)N=C(NCc2ccc(OC(F)(F)F)cc2)Nc2c(Oc3ccccc3Cl)cccc21] | 5-(2-chlorophenoxy)-3-((4- (trifluoromethoxy)benzyl)amino)- 4H-benzo[e][1,2,4]thiadiazine 1,1- dioxide |
| 1-117 | 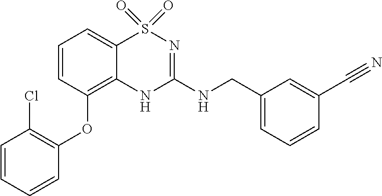 [SMILES: N#Cc1cccc(CNC2=NS(=O)(=O)c3cccc(Oc4ccccc4Cl)c3N2)c1] | 3-(((5-(2-chlorophenoxy)-1,1- dioxido-4H- benzo[e][1,2,4]thiadiazin-3- yl)amino)methyl)benzonitrile |
| 1-118 | 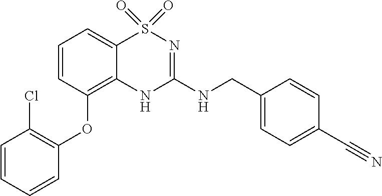 [SMILES: N#Cc1ccc(CNC2=NS(=O)(=O)c3cccc(Oc4ccccc4Cl)c3N2)cc1] | 4-(((5-(2-chlorophenoxy)-1,1- dioxido-4H- benzo[e][1,2,4]thiadiazin-3- yl)amino)methyl)benzonitrile |
| 1-119 | 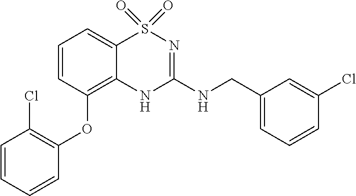 [SMILES: O=S1(=O)N=C(NCc2cccc(Cl)c2)Nc2c(Oc3ccccc3Cl)cccc21] | 3-((3-chlorobenzyl)amino)-5-(2- chlorophenoxy)-4H- benzo[e][1,2,4]thiadiazine 1,1- dioxide |
| 1-120 | 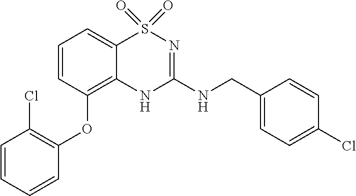 [SMILES: O=S1(=O)N=C(NCc2ccc(Cl)cc2)Nc2c(Oc3ccccc3Cl)cccc21] | 3-((4-chlorobenzyl)amino)-5-(2- chlorophenoxy)-4H- benzo[e][1,2,4]thiadiazine 1,1- dioxide |
| 1-121 | 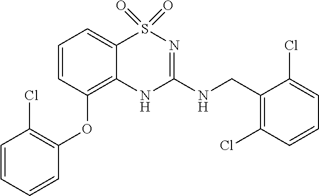 [SMILES: O=S1(=O)N=C(NCc2c(Cl)cccc2Cl)Nc2c(Oc3ccccc3Cl)cccc21] | 5-(2-chlorophenoxy)-3-((2,6- dichlorobenzyl)amino)-4H- benzo[e][1,2,4]thiadiazine 1,1- dioxide |
| 1-122 | 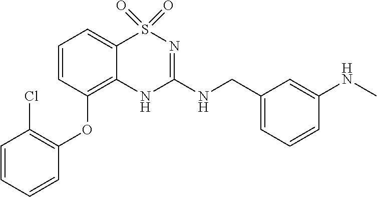 [SMILES: CNc1cccc(CNC2=NS(=O)(=O)c3cccc(Oc4ccccc4Cl)c3N2)c1] | 5-(2-chlorophenoxy)-3-((3- (methylamino)benzyl)amino)-4H- benzo[e][1,2,4]thiadiazine 1,1- dioxide |
| 1-123 | 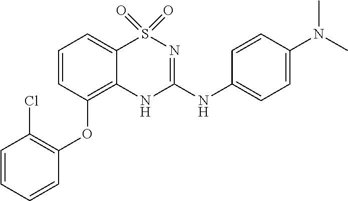 [SMILES: CN(C)c1ccc(NC2=NS(=O)(=O)c3cccc(Oc4ccccc4Cl)c3N2)cc1] | 5-(2-chlorophenoxy)-3-((4- (dimethylamino)phenyl)amino)- 4H-benzo[e][1,2,4]thiadiazine 1,1- dioxide |
| 1-124 | 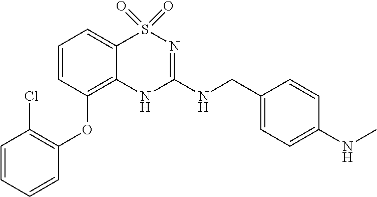 [SMILES: CNc1ccc(CNC2=NS(=O)(=O)c3cccc(Oc4ccccc4Cl)c3N2)cc1] | 5-(2-chlorophenoxy)-3-((4- (methylamino)benzyl)amino)-4H- benzo[e][1,2,4]thiadiazine 1,1- dioxide |
| 1-125 | 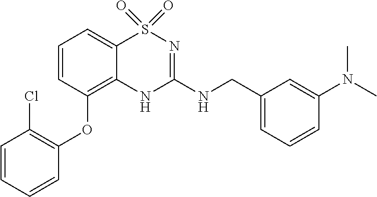 [SMILES: CN(C)c1cccc(CNC2=NS(=O)(=O)c3cccc(Oc4ccccc4Cl)c3N2)c1] | 5-(2-chlorophenoxy)-3-((3- (dimethylamino)benzyl)amino)- 4H-benzo[e][1,2,4]thiadiazine 1,1- dioxide |
| 1-126 | 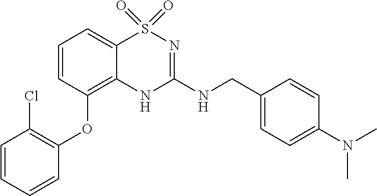 [SMILES: CN(C)c1ccc(CNC2=NS(=O)(=O)c3cccc(Oc4ccccc4Cl)c3N2)cc1] | 5-(2-chlorophenoxy)-3-((4- (dimethylamino)benzyl)amino)- 4H-benzo[e][1,2,4]thiadiazine 1,1- dioxide |
| 1-127 | 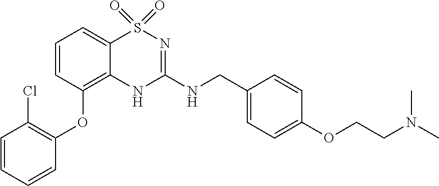 [SMILES: CN(C)CCOc1ccc(CNC2=NS(=O)(=O)c3cccc(Oc4ccccc4Cl)c3N2)cc1] | 5-(2-chlorophenoxy)-3-((4-(2- (dimethylamino)ethoxy)benzyl) amino)-4H-benzo[e] [1,2,4]thiadiazine 1,1-dioxide |
| 1-128 | 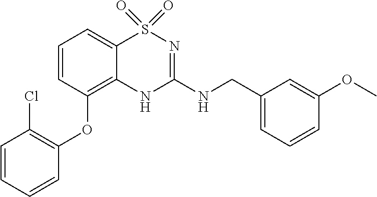 [SMILES: COc1cccc(CNC2=NS(=O)(=O)c3cccc(Oc4ccccc4Cl)c3N2)c1] | 5-(2-chlorophenoxy)-3-((3- methoxybenzyl)amino)-4H- benzo[e][1,2,4]thiadiazine 1,1- dioxide |
| 1-129 | 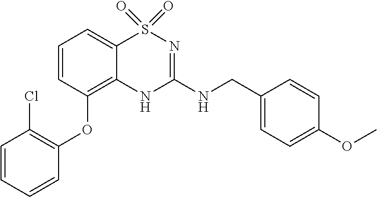 [SMILES: COc1ccc(CNC2=NS(=O)(=O)c3cccc(Oc4ccccc4Cl)c3N2)cc1] | 5-(2-chlorophenoxy)-3-((4- methoxybenzyl)amino)-4H- benzo[e][1,2,4]thiadiazine 1,1- dioxide |
| 1-130 | 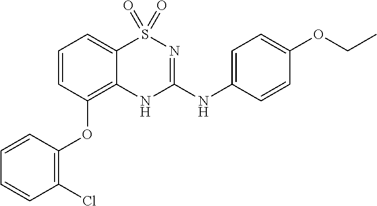 [SMILES: CCOc1ccc(NC2=NS(=O)(=O)c3cccc(Oc4ccccc4Cl)c3N2)cc1] | 5-(2-chlorophenoxy)-3-((4- ethoxyphenyl)amino)-4H- benzo[e][1,2,4]thiadiazine 1,1- dioxide |
| 1-131 | 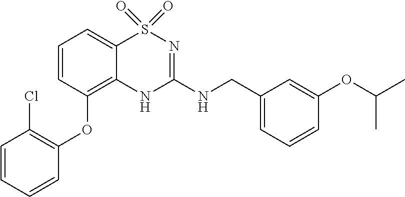 [SMILES: CC(C)Oc1cccc(CNC2=NS(=O)(=O)c3cccc(Oc4ccccc4Cl)c3N2)c1] | 5-(2-chlorophenoxy)-3-((3- isopropoxybenzyl)amino)-4H- benzo[e][1,2,4]thiadiazine 1,1- dioxide |
| 1-132 | 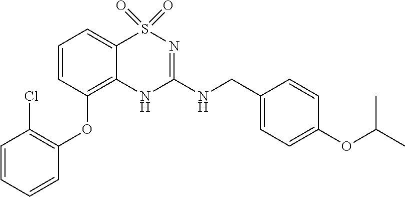 [SMILES: CC(C)Oc1ccc(CNC2=NS(=O)(=O)c3cccc(Oc4ccccc4Cl)c3N2)cc1] | 5-(2-chlorophenoxy)-3-((4- isopropoxybenzyl)amino)-4H- benzo[e][1,2,4]thiadiazine 1,1- dioxide |
| 1-133 | 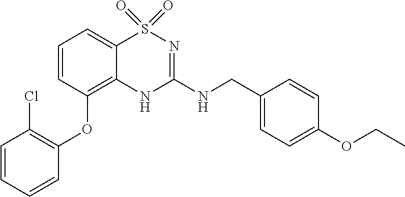 [SMILES: CCOc1ccc(CNC2=NS(=O)(=O)c3cccc(Oc4ccccc4Cl)c3N2)cc1] | 5-(2-chlorophenoxy)-3-((4- ethoxybenzyl)amino)-4H- benzo[e][1,2,4]thiadiazine 1,1- dioxide |
| 1-134 | 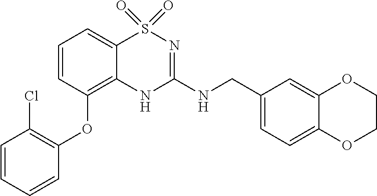 [SMILES: O=S1(=O)N=C(NCc2ccc3c(c2)OCCO3)Nc2c(Oc3ccccc3Cl)cccc21] | 5-(2-chlorophenoxy)-3-(((2,3- dihydrobenzo[b][1,4]dioxin-6- yl)methyl)amino)-4H- benzo[e][1,2,4]thiadiazine 1,1- dioxide |
| 1-135 | 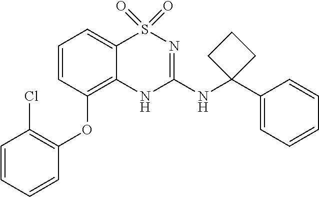 [SMILES: O=S1(=O)N=C(NC2(c3ccccc3)CCC2)Nc2c(Oc3ccccc3Cl)cccc21] | 5-(2-chlorophenoxy)-3-((1- phenylcyclobutyl)amino)-4H- benzo[e][1,2,4]thiadiazine 1,1- dioxide |
| 1-136 | 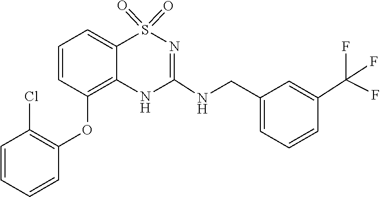 [SMILES: O=S1(=O)N=C(NCc2cccc(C(F)(F)F)c2)Nc2c(Oc3ccccc3Cl)cccc21] | 5-(2-chlorophenoxy)-3-((3- (trifluoromethyl)benzyl)amino)- 4H-benzo[e][1,2,4]thiadiazine 1,1- dioxide |
| 1-137 | 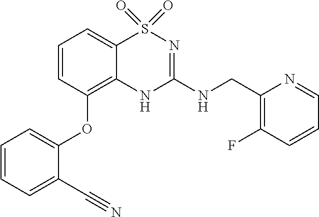 [SMILES: N#Cc1ccccc1Oc1cccc2c1NC(NCc1ncccc1F)=NS2(=O)=O] | 2-((3-(((3-fluoropyridin-2- yl)methyl)amino)-1,1-dioxido-4H- benzo[e][1,2,4]thiadiazin-5- yl)oxy)benzonitrile |
| 1-138 | 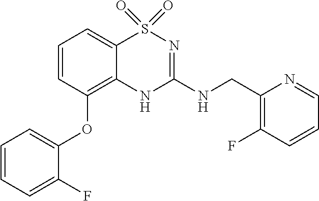 [SMILES: O=S1(=O)N=C(NCc2ncccc2F)Nc2c(Oc3ccccc3F)cccc21] | 5-(2-fluorophenoxy)-3-(((3- fluoropyridin-2-yl)methyl)amino)- 4H-benzo[e][1,2,4]thiadiazine 1,1- dioxide |
| 1-139 | 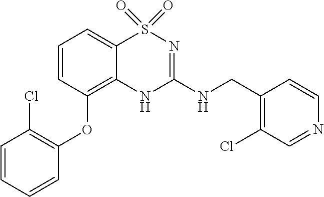 [SMILES: O=S1(=O)N=C(NCc2ccncc2Cl)Nc2c(Oc3ccccc3Cl)cccc21] | 5-(2-chlorophenoxy)-3-(((3- chloropyridin-4-yl)methyl)amino)- 4H-benzo[e][1,2,4]thiadiazine 1,1- dioxide |
| 1-140 | 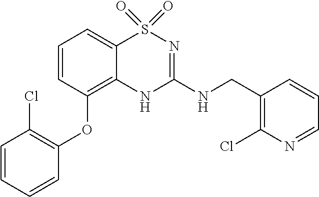 [SMILES: O=S1(=O)N=C(NCc2cccnc2Cl)Nc2c(Oc3ccccc3Cl)cccc21] | 5-(2-chlorophenoxy)-3-(((2- chloropyridin-3-yl)methyl)amino)- 4H-benzo[e][1,2,4]thiadiazine 1,1- dioxide |
| 1-141 | 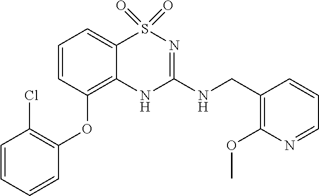 [SMILES: COc1ncccc1CNC1=NS(=O)(=O)c2cccc(Oc3ccccc3Cl)c2N1] | 5-(2-chlorophenoxy)-3-(((2- methoxypyridin-3- yl)methyl)amino)-4H- benzo[e][1,2,4]thiadiazine 1,1- dioxide |
| 1-142 | 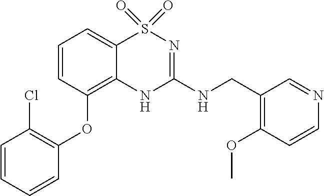 [SMILES: COc1ccncc1CNC1=NS(=O)(=O)c2cccc(Oc3ccccc3Cl)c2N1] | 5-(2-chlorophenoxy)-3-(((4- methoxypyridin-3- yl)methyl)amino)-4H- benzo[e][1,2,4]thiadiazine 1,1- dioxide |
| 1-143 | 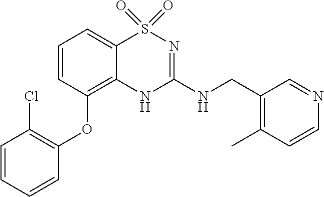 [SMILES: Cc1ccncc1CNC1=NS(=O)(=O)c2cccc(Oc3ccccc3Cl)c2N1] | 5-(2-chlorophenoxy)-3-(((4- methylpyridin-3-yl)methyl)amino)- 4H-benzo[e][1,2,4]thiadiazine 1,1- dioxide |
| 1-144 | 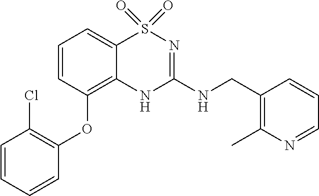 [SMILES: Cc1ncccc1CNC1=NS(=O)(=O)c2cccc(Oc3ccccc3Cl)c2N1] | 5-(2-chlorophenoxy)-3-(((2- methylpyridin-3-yl)methyl)amino)- 4H-benzo[e][1,2,4]thiadiazine 1,1- dioxide |
| 1-145 | 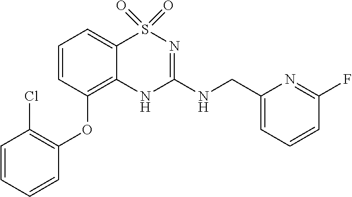 [SMILES: O=S1(=O)N=C(NCc2cccc(F)n2)Nc2c(Oc3ccccc3Cl)cccc21] | 5-(2-chlorophenoxy)-3-(((6- fluoropyridin-2-yl)methyl)amino)- 4H-benzo[e][1,2,4]thiadiazine 1,1- dioxide |
| 1-146 | 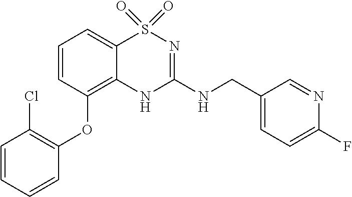 [SMILES: O=S1(=O)N=C(NCc2ccc(F)nc2)Nc2c(Oc3ccccc3Cl)cccc21] | 5-(2-chlorophenoxy)-3-(((6- fluoropyridin-3-yl)methyl)amino)- 4H-benzo[e][1,2,4]thiadiazine 1,1- dioxide |
| 1-147 | 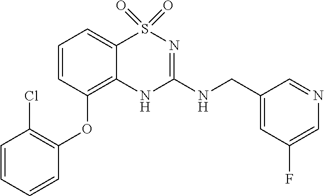 [SMILES: O=S1(=O)N=C(NCc2cncc(F)c2)Nc2c(Oc3ccccc3Cl)cccc21] | 5-(2-chlorophenoxy)-3-(((5- fluoropyridin-3-yl)methyl)amino)- 4H-benzo[e][1,2,4]thiadiazine 1,1- dioxide |
| 1-148 | 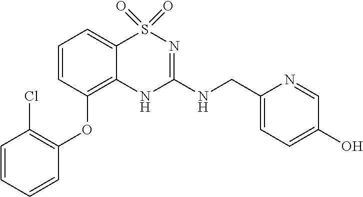 [SMILES: O=S1(=O)N=C(NCc2ccc(O)cn2)Nc2c(Oc3ccccc3Cl)cccc21] | 5-(2-chlorophenoxy)-3-(((5- hydroxypyridin-2- yl)methyl)amino)-4H- benzo[e][1,2,4]thiadiazine 1,1- dioxide |
| 1-149 | 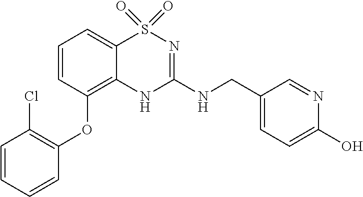 [SMILES: O=S1(=O)N=C(NCc2ccc(O)nc2)Nc2c(Oc3ccccc3Cl)cccc21] | 5-(2-chlorophenoxy)-3-(((6- hydroxypyridin-3- yl)methyl)amino)-4H- benzo[e][1,2,4]thiadiazine 1,1- dioxide |
| 1-150 | 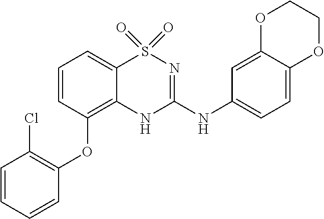 [SMILES: O=S1(=O)N=C(Nc2ccc3c(c2)OCCO3)Nc2c(Oc3ccccc3Cl)cccc21] | 5-(2-chlorophenoxy)-3-((2,3- dihydrobenzo[b][1,4]dioxin-6- yl)amino)-4H- benzo[e][1,2,4]thiadiazine 1,1- dioxide |
| 1-151 (racemic) | 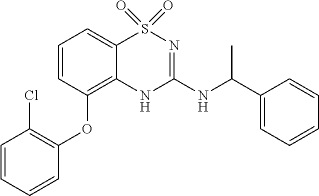 [SMILES: CC(NC1=NS(=O)(=O)c2cccc(Oc3ccccc3Cl)c2N1)c1ccccc1] | 5-(2-chlorophenoxy)-3-((1- phenylethyl)amino)-4H- benzo[e][1,2,4]thiadiazine 1,1- dioxide |
| 1-151 | 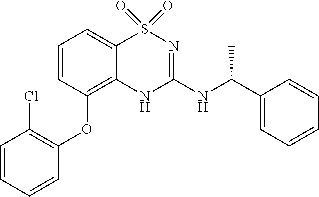 [SMILES: C[C@@H](NC1=NS(=O)(=O)c2cccc(Oc3ccccc3Cl)c2N1)c1ccccc1] | (R)-5-(2-chlorophenoxy)-3-((1- phenylethyl)amino)-4H- benzo[e][1,2,4]thiadiazine 1,1- dioxide |
| 1-152 | 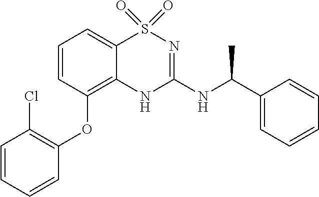 [SMILES: C[C@H](NC1=NS(=O)(=O)c2cccc(Oc3ccccc3Cl)c2N1)c1ccccc1] | (S)-5-(2-chlorophenoxy)-3-((1- phenylethyl)amino)-4H- benzo[e][1,2,4]thiadiazine 1,1- dioxide |
| 1-153 | 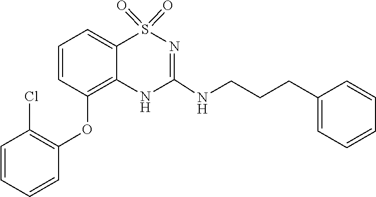 [SMILES: O=S1(=O)N=C(NCCCc2ccccc2)Nc2c(Oc3ccccc3Cl)cccc21] | 5-(2-chlorophenoxy)-3-((3- phenylpropyl)amino)-4H- benzo[e][1,2,4]thiadiazine 1,1- dioxide |
| 1-154 | 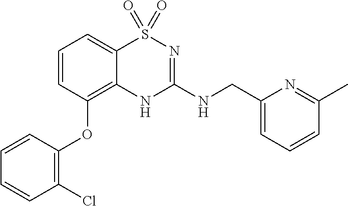 [SMILES: Cc1cccc(CNC2=NS(=O)(=O)c3cccc(Oc4ccccc4Cl)c3N2)n1] | 5-(2-chlorophenoxy)-3-(((6- methylpyridin-2-yl)methyl)amino)- 4H-benzo[e][1,2,4]thiadiazine 1,1- dioxide |
| 1-155 | 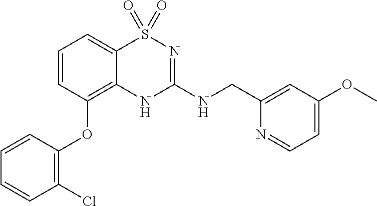 [SMILES: COc1ccnc(CNC2=NS(=O)(=O)c3cccc(Oc4ccccc4Cl)c3N2)c1] | 5-(2-chlorophenoxy)-3-(((4- methoxypyridin-2- yl)methyl)amino)-4H- benzo[e][1,2,4]thiadiazine 1,1- dioxide |
| 1-156 | 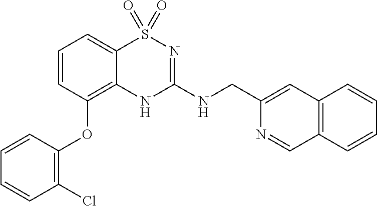 [SMILES: O=S1(=O)N=C(NCc2cc3ccccc3cn2)Nc2c(Oc3ccccc3Cl)cccc21] | 5-(2-chlorophenoxy)-3- ((isoquinolin-3-ylmethyl)amino)- 4H-benzo[e][1,2,4]thiadiazine 1,1- dioxide |
| 1-157 | 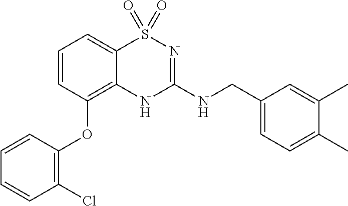 [SMILES: Cc1ccc(CNC2=NS(=O)(=O)c3cccc(Oc4ccccc4Cl)c3N2)cc1C] | 5-(2-chlorophenoxy)-3-((3,4- dimethylbenzyl)amino)-4H- benzo[e][1,2,4]thiadiazine 1,1- dioxide |
| 1-158 | 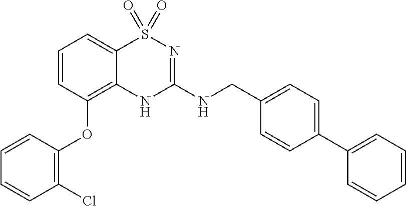 [SMILES: O=S1(=O)N=C(NCc2ccc(-c3ccccc3)cc2)Nc2c(Oc3ccccc3Cl)cccc21] | 3-(([1,1′-biphenyl]-4- ylmethyl)amino)-5-(2- chlorophenoxy)-4H- benzo[e][1,2,4]thiadiazine 1,1- dioxide |
| 1-159 | 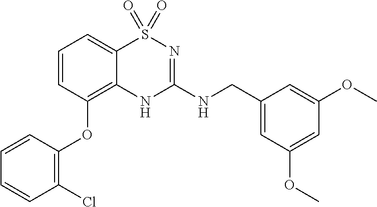 [SMILES: COc1cc(CNC2=NS(=O)(=O)c3cccc(Oc4ccccc4Cl)c3N2)cc(OC)c1] | 5-(2-chlorophenoxy)-3-((3,5- dimethoxybenzyl)amino)-4H- benzo[e][1,2,4]thiadiazine 1,1- dioxide |
| 1-160 | 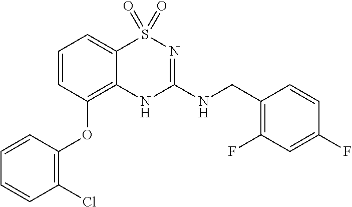 [SMILES: O=S1(=O)N=C(NCc2ccc(F)cc2F)Nc2c(Oc3ccccc3Cl)cccc21] | 5-(2-chlorophenoxy)-3-((2,4- difluorobenzyl)amino)-4H- benzo[e][1,2,4]thiadiazine 1,1- dioxide |
| 1-161 | 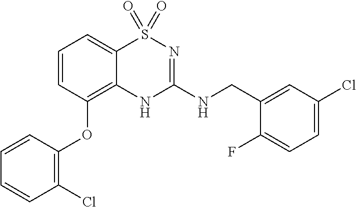 [SMILES: O=S1(=O)N=C(NCc2cc(Cl)ccc2F)Nc2c(Oc3ccccc3Cl)cccc21] | 3-((5-chloro-2- fluorobenzyl)amino)-5-(2- chlorophenoxy)-4H- benzo[e][1,2,4]thiadiazine 1,1- dioxide |
| 1-162 | 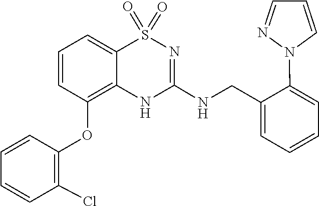 [SMILES: O=S1(=O)N=C(NCc2ccccc2-n2cccn2)Nc2c(Oc3ccccc3Cl)cccc21] | 3-((2-(1H-pyrazol-1- yl)benzyl)amino)-5-(2- chlorophenoxy)-4H- benzo[e][1,2,4]thiadiazine 1,1- dioxide |
| 1-163 | 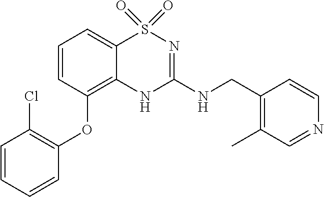 [SMILES: Cc1cnccc1CNC1=NS(=O)(=O)c2cccc(Oc3ccccc3Cl)c2N1] | 5-(2-chlorophenoxy)-3-(((3- methylpyridin-4-yl)methyl)amino)- 4H-benzo[e][1,2,4]thiadiazine 1,1- dioxide |
| 1-164 | 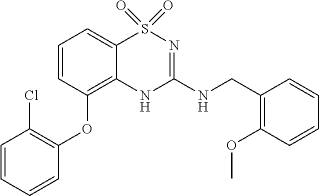 [SMILES: COc1ccccc1CNC1=NS(=O)(=O)c2cccc(Oc3ccccc3Cl)c2N1] | 5-(2-chlorophenoxy)-3-((2- methoxybenzyl)amino)-4H- benzo[e][1,2,4]thiadiazine 1,1- dioxide |
| 1-165 | 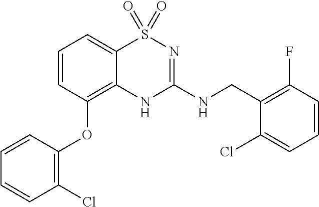 [SMILES: O=S1(=O)N=C(NCc2c(F)cccc2Cl)Nc2c(Oc3ccccc3Cl)cccc21] | 3-((2-chloro-6- fluorobenzyl)amino)-5-(2- chlorophenoxy)-4H- benzo[e][1,2,4]thiadiazine 1,1- dioxide |
| 1-166 | 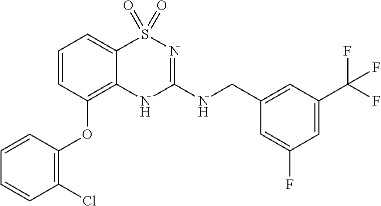 [SMILES: O=S1(=O)N=C(NCc2cc(F)cc(C(F)(F)F)c2)Nc2c(Oc3ccccc3Cl)cccc21] | 5-(2-chlorophenoxy)-3-((3-fluoro- 5-(trifluoromethyl)benzyl)amino)- 4H-benzo[e][1,2,4]thiadiazine 1,1- dioxide |
| 1-167 | 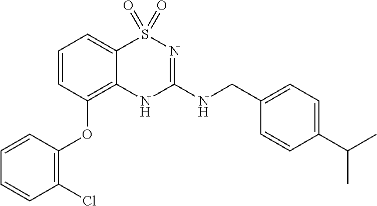 [SMILES: CC(C)c1ccc(CNC2=NS(=O)(=O)c3cccc(Oc4ccccc4Cl)c3N2)cc1] | 5-(2-chlorophenoxy)-3-((4- isopropylbenzyl)amino)-4H- benzo[e][1,2,4]thiadiazine 1,1- dioxide |
| 1-168 | 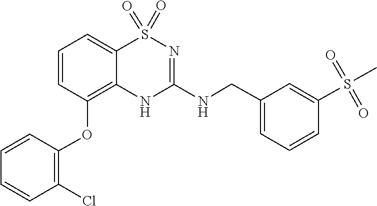 [SMILES: CS(=O)(=O)c1cccc(CNC2=NS(=O)(=O)c3cccc(Oc4ccccc4Cl)c3N2)c1] | 5-(2-chlorophenoxy)-3-((3- (methylsulfonyl)benzyl)amino)- 4H-benzo[e][1,2,4]thiadiazine 1,1- dioxide |
| 1-169 | 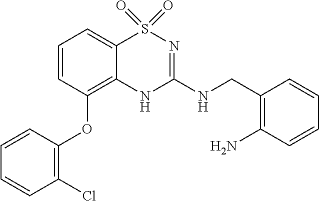 [SMILES: Nc1ccccc1CNC1=NS(=O)(=O)c2cccc(Oc3ccccc3Cl)c2N1] | 3-((2-aminobenzyl)amino)-5-(2- chlorophenoxy)-4H- benzo[e][1,2,4]thiadiazine 1,1- dioxide |
| 1-170 | 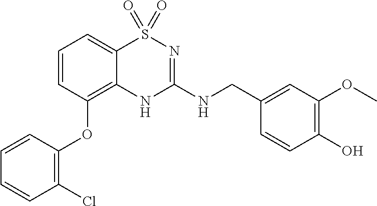 [SMILES: COc1cc(CNC2=NS(=O)(=O)c3cccc(Oc4ccccc4Cl)c3N2)ccc1O] | 5-(2-chlorophenoxy)-3-((4- hydroxy-3-methoxybenzyl)amino)- 4H-benzo[e][1,2,4]thiadiazine 1,1- dioxide |
| 1-171 | 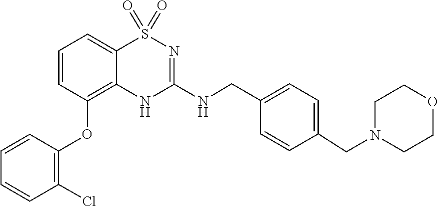 [SMILES: O=S1(=O)N=C(NCc2ccc(CN3CCOCC3)cc2)Nc2c(Oc3ccccc3Cl)cccc21] | 5-(2-chlorophenoxy)-3-((4- (morpholinomethyl)benzyl)amino)- 4H-benzo[e][1,2,4]thiadiazine 1,1-dioxide |
| 1-172 | 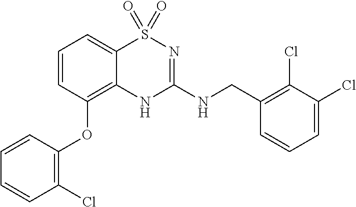 [SMILES: O=S1(=O)N=C(NCc2cccc(Cl)c2Cl)Nc2c(Oc3ccccc3Cl)cccc21] | 5-(2-chlorophenoxy)-3-((2,3- dichlorobenzyl)amino)-4H- benzo[e][1,2,4]thiadiazine 1,1- dioxide |
| 1-173 | 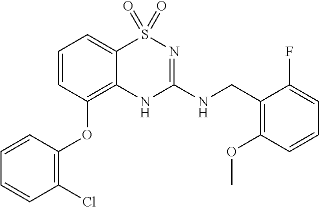 [SMILES: COc1cccc(F)c1CNC1=NS(=O)(=O)c2cccc(Oc3ccccc3Cl)c2N1] | 5-(2-chlorophenoxy)-3-((2-fluoro- 6-methoxybenzyl)amino)-4H- benzo[e][1,2,4]thiadiazine 1,1- dioxide |
| 1-174 | 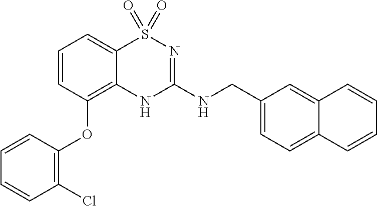 [SMILES: O=S1(=O)N=C(NCc2ccc3ccccc3c2)Nc2c(Oc3ccccc3Cl)cccc21] | 5-(2-chlorophenoxy)-3- ((naphthalen-2-ylmethyl)amino)- 4H-benzo[e][1,2,4]thiadiazine 1,1- dioxide |
| 1-175 | 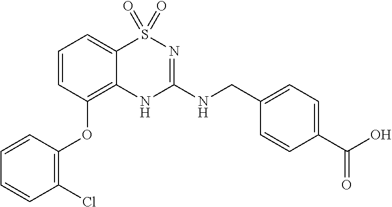 [SMILES: O=C(O)c1ccc(CNC2=NS(=O)(=O)c3cccc(Oc4ccccc4Cl)c3N2)cc1] | 4-(((5-(2-chlorophenoxy)-1,1- dioxido-4H- benzo[e][1,2,4]thiadiazin-3- yl)amino)methyl)benzoic acid |
| 1-176 | 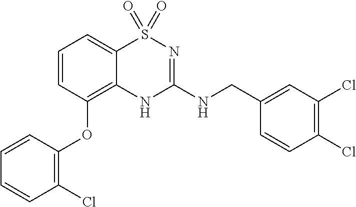 [SMILES: O=S1(=O)N=C(NCc2ccc(Cl)c(Cl)c2)Nc2c(Oc3ccccc3Cl)cccc21] | 5-(2-chlorophenoxy)-3-((3,4- dichlorobenzyl)amino)-4H- benzo[e][1,2,4]thiadiazine 1,1- dioxide |
| 1-177 | 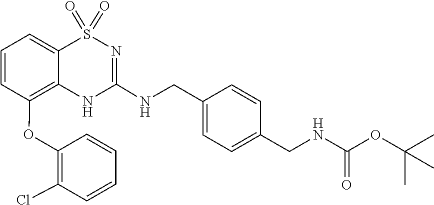 [SMILES: CC(C)(C)OC(=O)NCc1ccc(CNC2=NS(=O)(=O)c3cccc(Oc4ccccc4Cl)c3N2)cc1] | tert-butyl (4-(((5-(2- chlorophenoxy)-1,1-dioxido-4H- benzo[e][1,2,4]thiadiazin-3- yl)amino)methyl)benzyl)carbamate |
| 1-178 | 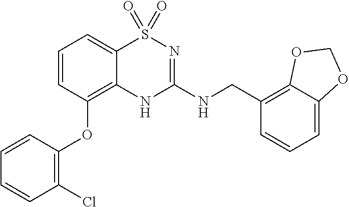 [SMILES: O=S1(=O)N=C(NCc2cccc3c2OCO3)Nc2c(Oc3ccccc3Cl)cccc21] | 3-((benzo[d][1,3]dioxol-4- ylmethyl)amino)-5-(2- chlorophenoxy)-4H- benzo[e][1,2,4]thiadiazine 1,1- dioxide |
| 1-179 | 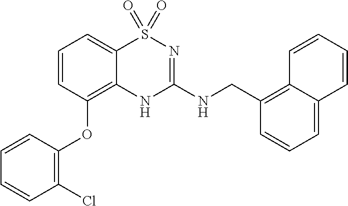 [SMILES: O=S1(=O)N=C(NCc2cccc3ccccc23)Nc2c(Oc3ccccc3Cl)cccc21] | 5-(2-chlorophenoxy)-3- ((naphthalen-1-ylmethyl)amino)- 4H-benzo[e][1,2,4]thiadiazine 1,1- dioxide |
| 1-180 | 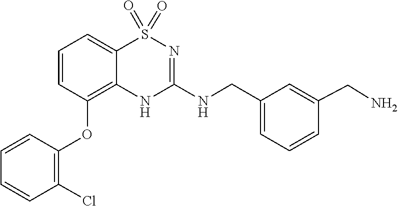 [SMILES: NCc1cccc(CNC2=NS(=O)(=O)c3cccc(Oc4ccccc4Cl)c3N2)c1] | 3-((3- (aminomethyl)benzyl)amino)-5-(2- chlorophenoxy)-4H- benzo[e][1,2,4]thiadiazine 1,1- dioxide |
| 1-181 | 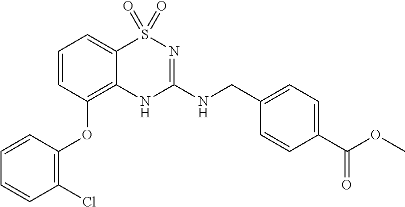 [SMILES: COC(=O)c1ccc(CNC2=NS(=O)(=O)c3cccc(Oc4ccccc4Cl)c3N2)cc1] | methyl 4-(((5-(2-chlorophenoxy)- 1,1-dioxido-4H- benzo[e][1,2,4]thiadiazin-3- yl)amino)methyl)benzoate |
| 1-182 | 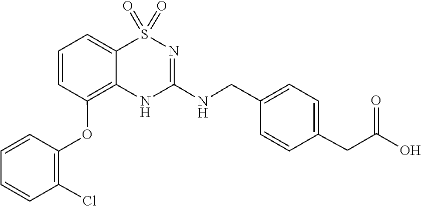 [SMILES: O=C(O)Cc1ccc(CNC2=NS(=O)(=O)c3cccc(Oc4ccccc4Cl)c3N2)cc1] | 2-(4-(((5-(2-chlorophenoxy)-1,1- dioxido-4H- benzo[e][1,2,4]thiadiazin-3- yl)amino)methyl)phenyl)acetic acid |
| 1-183 | 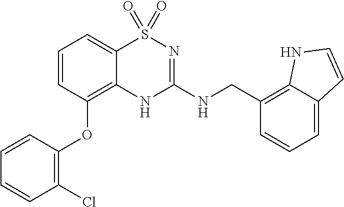 [SMILES: O=S1(=O)N=C(NCc2cccc3cc[nH]c23)Nc2c(Oc3ccccc3Cl)cccc21] | 3-(((1H-indol-7-yl)methyl)amino)- 5-(2-chlorophenoxy)-4H- benzo[e][1,2,4]thiadiazine 1,1- dioxide |
| 1-184 | 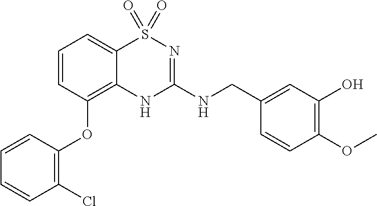 [SMILES: COc1ccc(CNC2=NS(=O)(=O)c3cccc(Oc4ccccc4Cl)c3N2)cc1O] | 5-(2-chlorophenoxy)-3-((3- hydroxy-4-methoxybenzyl)amino)- 4H-benzo[e][1,2,4]thiadiazine 1,1- dioxide |
| 1-185 | 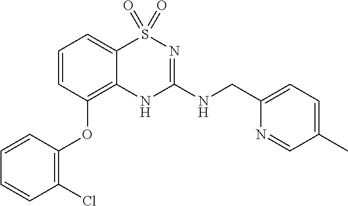 [SMILES: Cc1ccc(CNC2=NS(=O)(=O)c3cccc(Oc4ccccc4Cl)c3N2)nc1] | 5-(2-chlorophenoxy)-3-(((5- methylpyridin-2-yl)methyl)amino)- 4H-benzo[e][1,2,4]thiadiazine 1,1- dioxide |
| 1-186 | 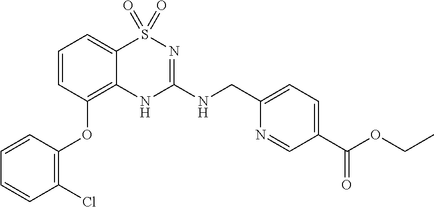 [SMILES: CCOC(=O)c1ccc(CNC2=NS(=O)(=O)c3cccc(Oc4ccccc4Cl)c3N2)nc1] | ethyl 6-(((5-(2-chlorophenoxy)- 1,1-dioxido-4H- benzo[e][1,2,4]thiadiazin-3- yl)amino)methyl)nicotinate |
| 1-187 | 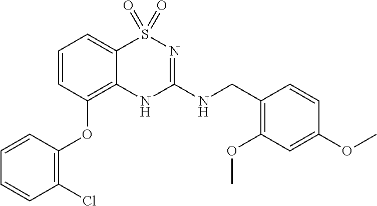 [SMILES: COc1ccc(CNC2=NS(=O)(=O)c3cccc(Oc4ccccc4Cl)c3N2)c(OC)c1] | 5-(2-chlorophenoxy)-3-((2,4- dimethoxybenzyl)amino)-4H- benzo[e][1,2,4]thiadiazine 1,1- dioxide |
| 1-188 | 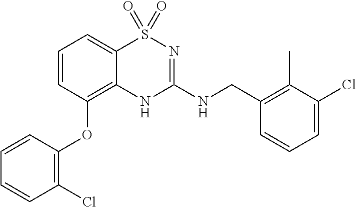 [SMILES: Cc1c(Cl)cccc1CNC1=NS(=O)(=O)c2cccc(Oc3ccccc3Cl)c2N1] | 3-((3-chloro-2- methylbenzyl)amino)-5-(2- chlorophenoxy)-4H- benzo[e][1,2,4]thiadiazine 1,1- dioxide |
| 1-189 | 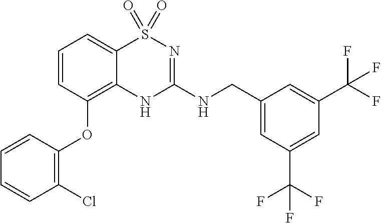 [SMILES: O=S1(=O)N=C(NCc2cc(C(F)(F)F)cc(C(F)(F)F)c2)Nc2c(Oc3ccccc3Cl)cccc21] | 3-((3,5- bis(trifluoromethyl)benzyl)amino)- 5-(2-chlorophenoxy)-4H- benzo[e][1,2,4]thiadiazine 1,1- dioxide |
| 1-190 | 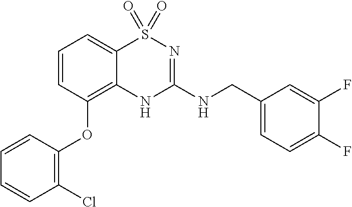 [SMILES: O=S1(=O)N=C(NCc2ccc(F)c(F)c2)Nc2c(Oc3ccccc3Cl)cccc21] | 5-(2-chlorophenoxy)-3-((3,4- difluorobenzyl)amino)-4H- benzo[e][1,2,4]thiadiazine 1,1- dioxide |
| 1-191 | 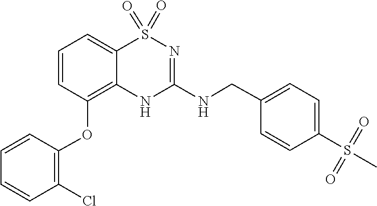 [SMILES: CS(=O)(=O)c1ccc(CNC2=NS(=O)(=O)c3cccc(Oc4ccccc4Cl)c3N2)cc1] | 5-(2-chlorophenoxy)-3-((4- (methylsulfonyl)benzyl)amino)- 4H-benzo[e][1,2,4]thiadiazine 1,1- dioxide |
| 1-192 | 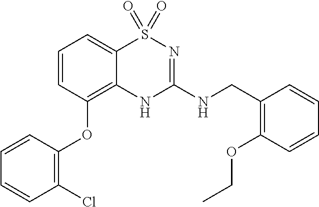 [SMILES: CCOc1ccccc1CNC1=NS(=O)(=O)c2cccc(Oc3ccccc3Cl)c2N1] | 5-(2-chlorophenoxy)-3-((2- ethoxybenzyl)amino)-4H- benzo[e][1,2,4]thiadiazine 1,1- dioxide |
| 1-193 | 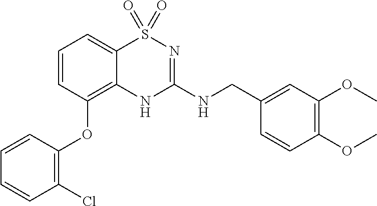 [SMILES: COc1ccc(CNC2=NS(=O)(=O)c3cccc(Oc4ccccc4Cl)c3N2)cc1OC] | 5-(2-chlorophenoxy)-3-((3,4- dimethoxybenzyl)amino)-4H- benzo[e][1,2,4]thiadiazine 1,1- dioxide |
| 1-194 | 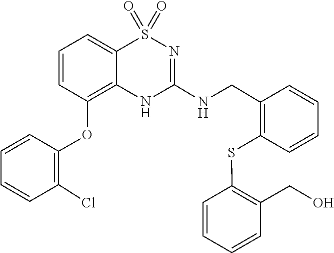 [SMILES: O=S1(=O)N=C(NCc2ccccc2Sc2ccccc2CO)Nc2c(Oc3ccccc3Cl)cccc21] | 5-(2-chlorophenoxy)-3-((2-((2- (hydroxymethyl)phenyl)thio) benzyl)amino)-4H- benzo[e][1,2,4]thiadiazine 1,1- dioxide |
| 1-195 | 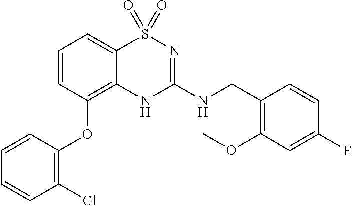 [SMILES: COc1cc(F)ccc1CNC1=NS(=O)(=O)c2cccc(Oc3ccccc3Cl)c2N1] | 5-(2-chlorophenoxy)-3-((4-fluoro- 2-methoxybenzyl)amino)-4H- benzo[e][1,2,4]thiadiazine 1,1- dioxide |
| 1-196 | 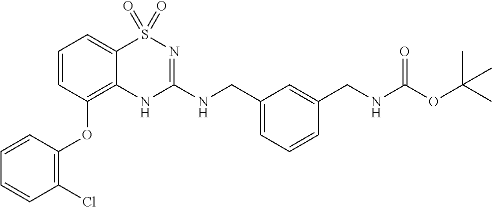 [SMILES: CC(C)(C)OC(=O)NCc1cccc(CNC2=NS(=O)(=O)c3cccc(Oc4ccccc4Cl)c3N2)c1] | tert-butyl (3-(((5-(2- chlorophenoxy)-1,1-dioxido-4H- benzo[e][1,2,4]thiadiazin-3- yl)amino)methyl)benzyl)carbamate |
| 1-197 | 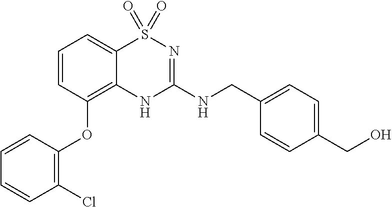 [SMILES: O=S1(=O)N=C(NCc2ccc(CO)cc2)Nc2c(Oc3ccccc3Cl)cccc21] | 5-(2-chlorophenoxy)-3-((4- (hydroxymethyl)benzyl)amino)- 4H-benzo[e][1,2,4]thiadiazine 1,1- dioxide |
| 1-198 | 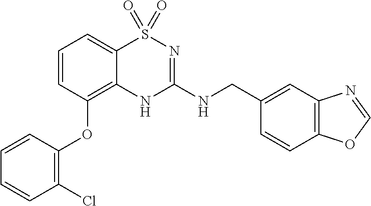 [SMILES: O=S1(=O)N=C(NCc2ccc3ocnc3c2)Nc2c(Oc3ccccc3Cl)cccc21] | 3-((benzo[d]oxazol-5- ylmethyl)amino)-5-(2- chlorophenoxy)-4H- benzo[e][1,2,4]thiadiazine 1,1- dioxide |
| 1-199 | 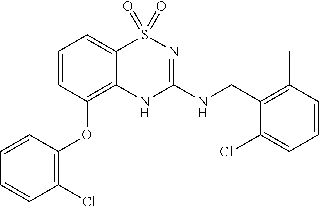 [SMILES: Cc1cccc(Cl)c1CNC1=NS(=O)(=O)c2cccc(Oc3ccccc3Cl)c2N1] | 3-((2-chloro-6- methylbenzyl)amino)-5-(2- chlorophenoxy)-4H- benzo[e][1,2,4]thiadiazine 1,1- dioxide |
| 1-200 | 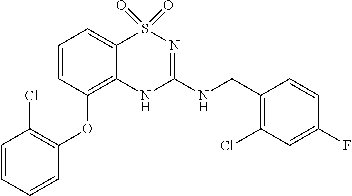 [SMILES: O=S1(=O)N=C(NCc2ccc(F)cc2Cl)Nc2c(Oc3ccccc3Cl)cccc21] | 3-((2-chloro-4- fluorobenzyl)amino)-5-(2- chlorophenoxy)-4H- benzo[e][1,2,4]thiadiazine 1,1- dioxide |
| 1-201 | 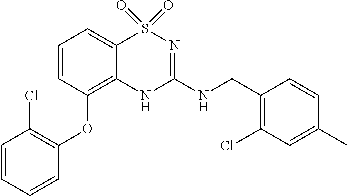 [SMILES: Cc1ccc(CNC2=NS(=O)(=O)c3cccc(Oc4ccccc4Cl)c3N2)c(Cl)c1] | 3-((2-chloro-4- methylbenzyl)amino)-5-(2- chlorophenoxy)-4H- benzo[e][1,2,4]thiadiazine 1,1- dioxide |
| 1-202 | 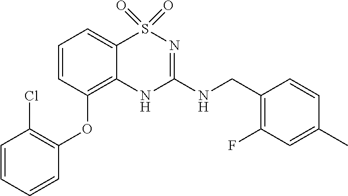 [SMILES: Cc1ccc(CNC2=NS(=O)(=O)c3cccc(Oc4ccccc4Cl)c3N2)c(F)c1] | 5-(2-chlorophenoxy)-3-((2-fluoro- 4-methylbenzyl)amino)-4H- benzo[e][1,2,4]thiadiazine 1,1- dioxide |
| 1-203 | 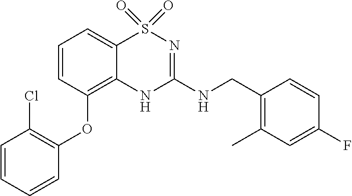 [SMILES: Cc1cc(F)ccc1CNC1=NS(=O)(=O)c2cccc(Oc3ccccc3Cl)c2N1] | 5-(2-chlorophenoxy)-3-((4-fluoro- 2-methylbenzyl)amino)-4H- benzo[e][1,2,4]thiadiazine 1,1- dioxide |
| 1-204 | 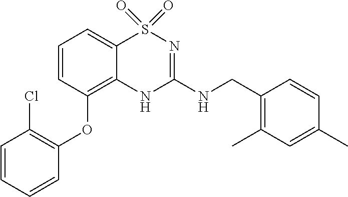 [SMILES: Cc1ccc(CNC2=NS(=O)(=O)c3cccc(Oc4ccccc4Cl)c3N2)c(C)c1] | 5-(2-chlorophenoxy)-3-((2,4- dimethylbenzyl)amino)-4H- benzo[e][1,2,4]thiadiazine 1,1- dioxide |
| 1-205 | 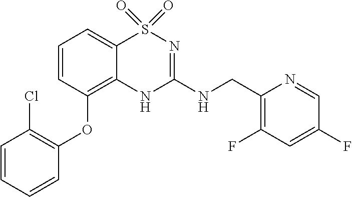 [SMILES: O=S1(=O)N=C(NCc2ncc(F)cc2F)Nc2c(Oc3ccccc3Cl)cccc21] | 5-(2-chlorophenoxy)-3-(((3,5- difluoropyridin-2- yl)methyl)amino)-4H- benzo[e][1,2,4]thiadiazine 1,1- dioxide |
| 1-206 | 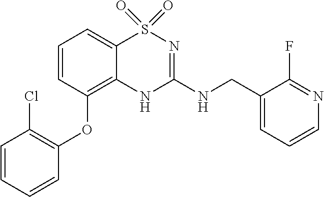 [SMILES: O=S1(=O)N=C(NCc2cccnc2F)Nc2c(Oc3ccccc3Cl)cccc21] | 5-(2-chlorophenoxy)-3-(((2- fluoropyridin-3-yl)methyl)amino)- 4H-benzo[e][1,2,4]thiadiazine 1,1- dioxide |
| 1-207 | 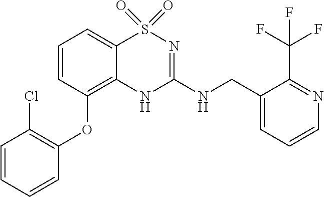 [SMILES: O=S1(=O)N=C(NCc2cccnc2C(F)(F)F)Nc2c(Oc3ccccc3Cl)cccc21] | 5-(2-chlorophenoxy)-3-(((2- (trifluoromethyl)pyridin-3- yl)methyl)amino)-4H- benzo[e][1,2,4]thiadiazine 1,1- dioxide |
| 1-208 | 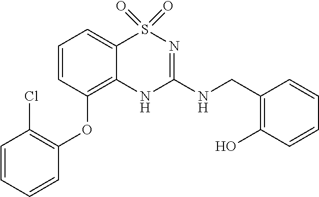 [SMILES: O=S1(=O)N=C(NCc2ccccc2O)Nc2c(Oc3ccccc3Cl)cccc21] | 5-(2-chlorophenoxy)-3-((2- hydroxybenzyl)amino)-4H- benzo[e][1,2,4]thiadiazine 1,1- dioxide |
| 1-209 | 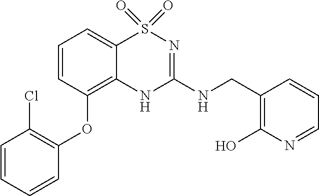 [SMILES: O=S1(=O)N=C(NCc2cccnc2O)Nc2c(Oc3ccccc3Cl)cccc21] | 5-(2-chlorophenoxy)-3-(((2- hydroxypyridin-3- yl)methyl)amino)-4H- benzo[e][1,2,4]thiadiazine 1,1- dioxide |
| 1-210 | 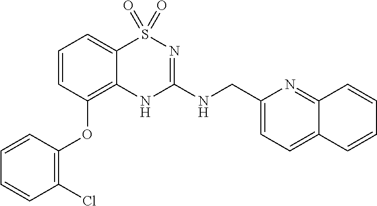 [SMILES: O=S1(=O)N=C(NCc2ccc3ccccc3n2)Nc2c(Oc3ccccc3Cl)cccc21] | 5-(2-chlorophenoxy)-3-((quinolin- 2-ylmethyl)amino)-4H- benzo[e][1,2,4]thiadiazine 1,1- dioxide |
| 1-211 | 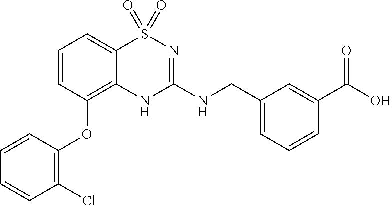 [SMILES: O=C(O)c1cccc(CNC2=NS(=O)(=O)c3cccc(Oc4ccccc4Cl)c3N2)c1] | 3-(((5-(2-chlorophenoxy)-1,1- dioxido-4H- benzo[e][1,2,4]thiadiazin-3- yl)amino)methyl)benzoic acid |
| 1-212 | 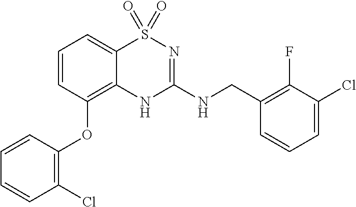 [SMILES: O=S1(=O)N=C(NCc2cccc(Cl)c2F)Nc2c(Oc3ccccc3Cl)cccc21] | 3-((3-chloro-2- fluorobenzyl)amino)-5-(2- chlorophenoxy)-4H- benzo[e][1,2,4]thiadiazine 1,1- dioxide |
| 1-213 | 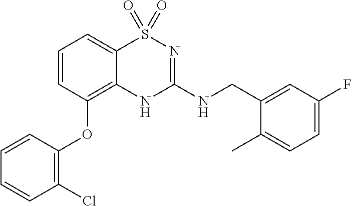 [SMILES: Cc1ccc(F)cc1CNC1=NS(=O)(=O)c2cccc(Oc3ccccc3Cl)c2N1] | 5-(2-chlorophenoxy)-3-((5-fluoro- 2-methylbenzyl)amino)-4H- benzo[e][1,2,4]thiadiazine 1,1- dioxide |
| 1-214 | 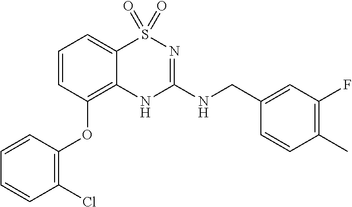 [SMILES: Cc1ccc(CNC2=NS(=O)(=O)c3cccc(Oc4ccccc4Cl)c3N2)cc1F] | 5-(2-chlorophenoxy)-3-((3-fluoro- 4-methylbenzyl)amino)-4H- benzo[e][1,2,4]thiadiazine 1,1- dioxide |
| 1-215 | 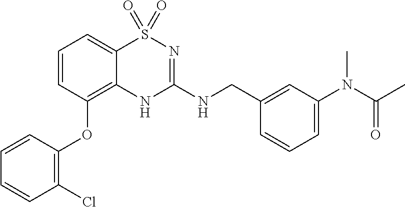 [SMILES: CC(=O)N(C)c1cccc(CNC2=NS(=O)(=O)c3cccc(Oc4ccccc4Cl)c3N2)c1] | N-(3-(((5-(2-chlorophenoxy)-1,1- dioxido-4H- benzo[e][1,2,4]thiadiazin-3- yl)amino)methyl)phenyl)-N- methylacetamide |
| 1-216 | 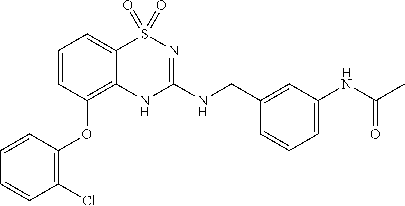 [SMILES: CC(=O)Nc1cccc(CNC2=NS(=O)(=O)c3cccc(Oc4ccccc4Cl)c3N2)c1] | N-(3-(((5-(2-chlorophenoxy)-1,1- dioxido-4H- benzo[e][1,2,4]thiadiazin-3- yl)amino)methyl)phenyl)acetamide |
| 1-217 | 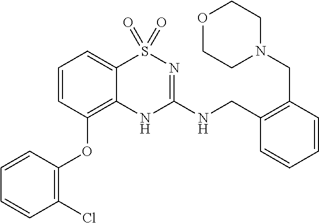 [SMILES: O=S1(=O)N=C(NCc2ccccc2CN2CCOCC2)Nc2c(Oc3ccccc3Cl)cccc21] | 5-(2-chlorophenoxy)-3-((2- (morpholinomethyl)benzyl)amino)- 4H-benzo[e][1,2,4]thiadiazine 1,1-dioxide |
| 1-218 | 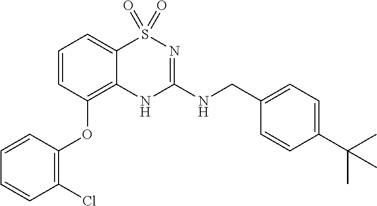 [SMILES: CC(C)(C)c1ccc(CNC2=NS(=O)(=O)c3cccc(Oc4ccccc4Cl)c3N2)cc1] | 3-((4-(tert-butyl)benzyl)amino)-5- (2-chlorophenoxy)-4H- benzo[e][1,2,4]thiadiazine 1,1- dioxide |
| 1-219 | 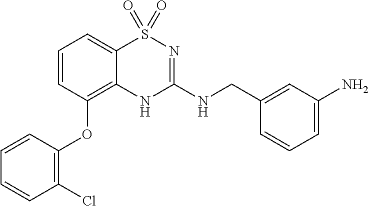 [SMILES: Nc1cccc(CNC2=NS(=O)(=O)c3cccc(Oc4ccccc4Cl)c3N2)c1] | 3-((3-aminobenzyl)amino)-5-(2- chlorophenoxy)-4H- benzo[e][1,2,4]thiadiazine 1,1- dioxide |
| 1-220 | 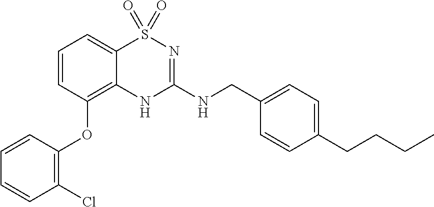 [SMILES: CCCCc1ccc(CNC2=NS(=O)(=O)c3cccc(Oc4ccccc4Cl)c3N2)cc1] | 3-((4-butylbenzyl)amino)-5-(2- chlorophenoxy)-4H- benzo[e][1,2,4]thiadiazine 1,1- dioxide |
| 1-221 | 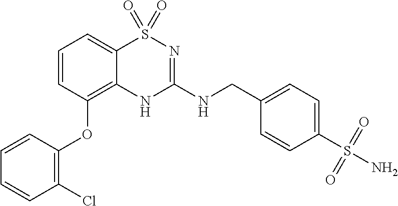 [SMILES: NS(=O)(=O)c1ccc(CNC2=NS(=O)(=O)c3cccc(Oc4ccccc4Cl)c3N2)cc1] | 4-(((5-(2-chlorophenoxy)-1,1- dioxido-4H- benzo[e][1,2,4]thiadiazin-3- yl)amino)methyl) benzenesulfonamide |
| 1-222 | 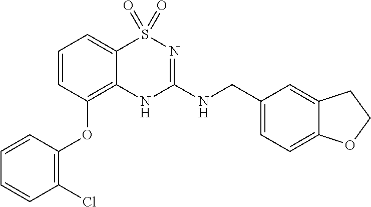 [SMILES: O=S1(=O)N=C(NCc2ccc3c(c2)CCO3)Nc2c(Oc3ccccc3Cl)cccc21] | 5-(2-chlorophenoxy)-3-(((2,3- dihydrobenzofuran-5- yl)methyl)amino)-4H- benzo[e][1,2,4]thiadiazine 1,1- dioxide |
| 1-223 | 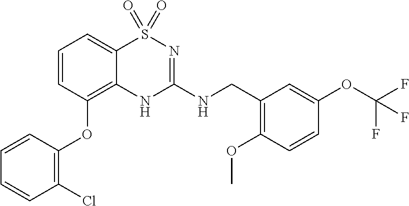 [SMILES: COc1ccc(OC(F)(F)F)cc1CNC1=NS(=O)(=O)c2cccc(Oc3ccccc3Cl)c2N1] | 5-(2-chlorophenoxy)-3-((2- methoxy-5- (trifluoromethoxy)benzyl)amino)- 4H-benzo[e][1,2,4]thiadiazine 1,1- dioxide |
| 1-224 | 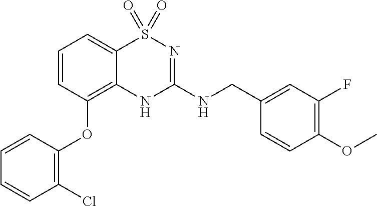 [SMILES: COc1ccc(CNC2=NS(=O)(=O)c3cccc(Oc4ccccc4Cl)c3N2)cc1F] | 5-(2-chlorophenoxy)-3-((3-fluoro- 4-methoxybenzyl)amino)-4H- benzo[e][1,2,4]thiadiazine 1,1- dioxide |
| 1-225 | 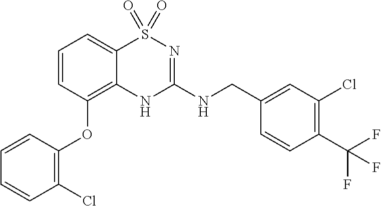 [SMILES: O=S1(=O)N=C(NCc2ccc(C(F)(F)F)c(Cl)c2)Nc2c(Oc3ccccc3Cl)cccc21] | 3-((3-chloro-4- (trifluoromethyl)benzyl)amino)-5- (2-chlorophenoxy)-4H- benzo[e][1,2,4]thiadiazine 1,1- dioxide |
| 1-226 | 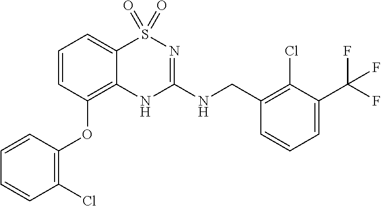 [SMILES: O=S1(=O)N=C(NCc2cccc(C(F)(F)F)c2Cl)Nc2c(Oc3ccccc3Cl)cccc21] | 3-((2-chloro-3- (trifluoromethyl)benzyl)amino)-5- (2-chlorophenoxy)-4H- benzo[e][1,2,4]thiadiazine 1,1- dioxide |
| 1-227 | 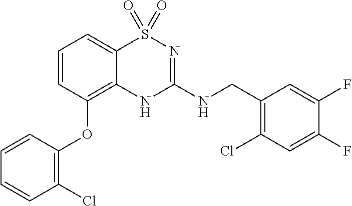 [SMILES: O=S1(=O)N=C(NCc2cc(F)c(F)cc2Cl)Nc2c(Oc3ccccc3Cl)cccc21] | 3-((2-chloro-4,5- difluorobenzyl)amino)-5-(2- chlorophenoxy)-4H- benzo[e][1,2,4]thiadiazine 1,1- dioxide |
| 1-228 | 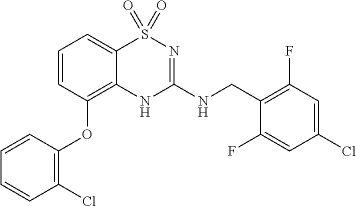 [SMILES: O=S1(=O)N=C(NCc2c(F)cc(Cl)cc2F)Nc2c(Oc3ccccc3Cl)cccc21] | 3-((4-chloro-2,6- difluorobenzyl)amino)-5-(2- chlorophenoxy)-4H- benzo[e][1,2,4]thiadiazine 1,1- dioxide |
| 1-229 | 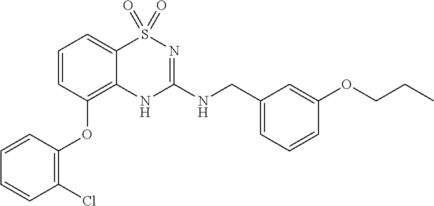 [SMILES: CCCOc1cccc(CNC2=NS(=O)(=O)c3cccc(Oc4ccccc4Cl)c3N2)c1] | 5-(2-chlorophenoxy)-3-((3- propoxybenzyl)amino)-4H- benzo[e][1,2,4]thiadiazine 1,1- dioxide |
| 1-230 | 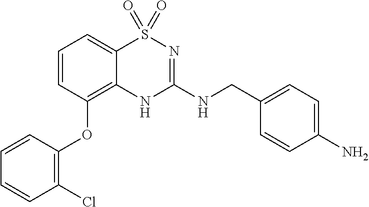 [SMILES: Nc1ccc(CNC2=NS(=O)(=O)c3cccc(Oc4ccccc4Cl)c3N2)cc1] | 3-((4-aminobenzyl)amino)-5-(2- chlorophenoxy)-4H- benzo[e][1,2,4]thiadiazine 1,1- dioxide |
| 1-231 | 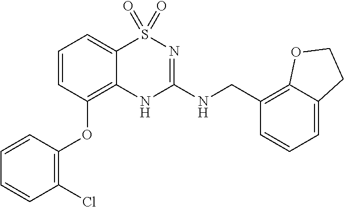 [SMILES: O=S1(=O)N=C(NCc2cccc3c2OCC3)Nc2c(Oc3ccccc3Cl)cccc21] | 5-(2-chlorophenoxy)-3-(((2,3- dihydrobenzofuran-7- yl)methyl)amino)-4H- benzo[e][1,2,4]thiadiazine 1,1- dioxide |
| 1-232 | 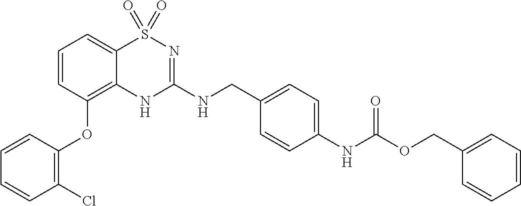 [SMILES: O=C(Nc1ccc(CNC2=NS(=O)(=O)c3cccc(Oc4ccccc4Cl)c3N2)cc1)OCc1ccccc1] | benzyl (4-(((5-(2-chlorophenoxy)- 1,1-dioxido-4H- benzo[e][1,2,4]thiadiazin-3- yl)amino)methyl)phenyl)carbamate |
| 1-233 | 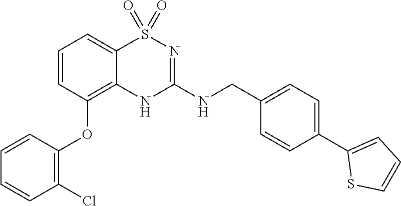 [SMILES: O=S1(=O)N=C(NCc2ccc(-c3cccs3)cc2)Nc2c(Oc3ccccc3Cl)cccc21] | 5-(2-chlorophenoxy)-3-((4- (thiophen-2-yl)benzyl)amino)-4H- benzo[e][1,2,4]thiadiazine 1,1- dioxide |
| 1-234 | 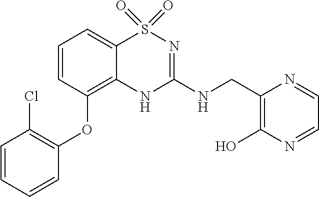 [SMILES: O=S1(=O)N=C(NCc2nccnc2O)Nc2c(Oc3ccccc3Cl)cccc21] | 5-(2-chlorophenoxy)-3-(((3- hydroxypyrazin-2- yl)methyl)amino)-4H- benzo[e][1,2,4]thiadiazine 1,1- dioxide |
| 1-235 | 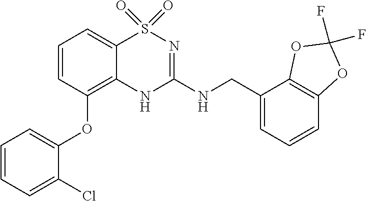 [SMILES: O=S1(=O)N=C(NCc2cccc3c2OC(F)(F)O3)Nc2c(Oc3ccccc3Cl)cccc21] | 5-(2-chlorophenoxy)-3-(((2,2- difluorobenzo[d][1,3]dioxol-4- yl)methyl)amino)-4H- benzo[e][1,2,4]thiadiazine 1,1- dioxide |
| 1-236 (racemic) | 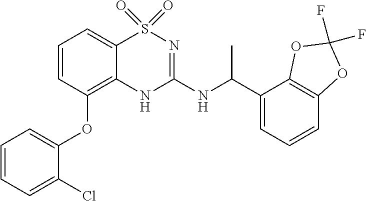 [SMILES: CC(NC1=NS(=O)(=O)c2cccc(Oc3ccccc3Cl)c2N1)c1cccc2c1OC(F)(F)O2] | 5-(2-chlorophenoxy)-3-((1-(2,2- difluorobenzo[d][1,3]dioxol-4- yl)ethyl)amino)-4H- benzo[e][1,2,4]thiadiazine 1,1- dioxide |
| 1-236 | 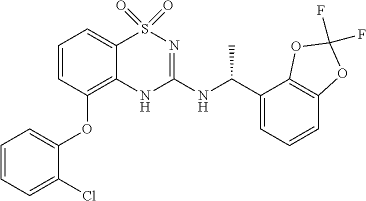 [SMILES: C[C@@H](NC1=NS(=O)(=O)c2cccc(Oc3ccccc3Cl)c2N1)c1cccc2c1OC(F)(F)O2] | (R)-5-(2-chlorophenoxy)-3-((1- (2,2-difluorobenzo[d][1,3]dioxol- 4-yl)ethyl)amino)-4H- benzo[e][1,2,4]thiadiazine 1,1- dioxide |
| 1-237 | 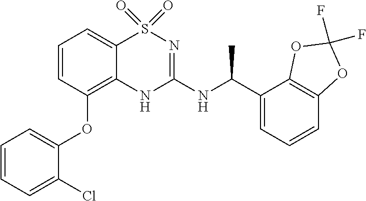 [SMILES: C[C@H](NC1=NS(=O)(=O)c2cccc(Oc3ccccc3Cl)c2N1)c1cccc2c1OC(F)(F)O2] | (S)-5-(2-chlorophenoxy)-3-((1- (2,2-difluorobenzo[d][1,3]dioxol- 4-yl)ethyl)amino)-4H- benzo[e][1,2,4]thiadiazine 1,1- dioxide |
| 1-238 | 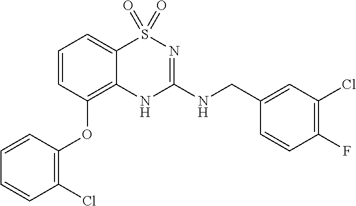 [SMILES: O=S1(=O)N=C(NCc2ccc(F)c(Cl)c2)Nc2c(Oc3ccccc3Cl)cccc21] | 3-((3-chloro-4- fluorobenzyl)amino)-5-(2- chlorophenoxy)-4H- benzo[e][1,2,4]thiadiazine 1,1- dioxide |
| 1-239 | 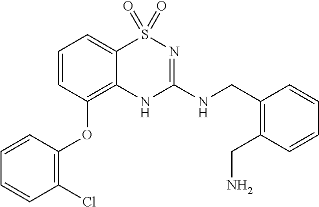 [SMILES: NCc1ccccc1CNC1=NS(=O)(=O)c2cccc(Oc3ccccc3Cl)c2N1] | 3-((2- (aminomethyl)benzyl)amino)-5-(2- chlorophenoxy)-4H- benzo[e][1,2,4]thiadiazine 1,1- dioxide |
| 1-240 | 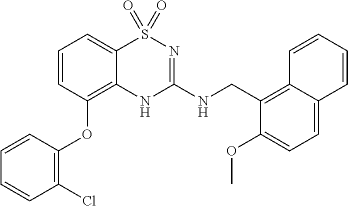 [SMILES: COc1ccc2ccccc2c1CNC1=NS(=O)(=O)c2cccc(Oc3ccccc3Cl)c2N1] | 5-(2-chlorophenoxy)-3-(((2- methoxynaphthalen-1- yl)methyl)amino)-4H- benzo[e][1,2,4]thiadiazine 1,1- dioxide |
| 1-241 | 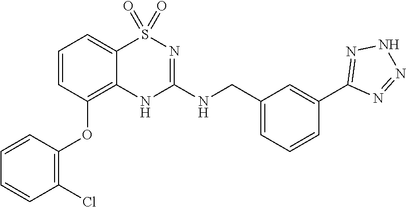 [SMILES: O=S1(=O)N=C(NCc2cccc(-c3nn[nH]n3)c2)Nc2c(Oc3ccccc3Cl)cccc21] | 3-((3-(2H-tetrazol-5- yl)benzyl)amino)-5-(2- chlorophenoxy)-4H- benzo[e][1,2,4]thiadiazine 1,1- dioxide |
| 1-242 | 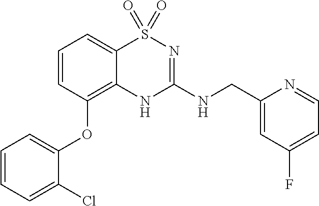 [SMILES: O=S1(=O)N=C(NCc2cc(F)ccn2)Nc2c(Oc3ccccc3Cl)cccc21] | 5-(2-chlorophenoxy)-3-(((4- fluoropyridin-2-yl)methyl)amino)- 4H-benzo[e][1,2,4]thiadiazine 1,1- dioxide |
| 1-243 | 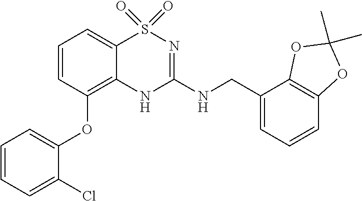 [SMILES: CC1(C)Oc2cccc(CNC3=NS(=O)(=O)c4cccc(Oc5ccccc5Cl)c4N3)c2O1] | 5-(2-chlorophenoxy)-3-(((2,2- dimethylbenzo[d][1,3]dioxol-4- yl)methyl)amino)-4H- benzo[e][1,2,4]thiadiazine 1,1- dioxide |
| 1-244 | 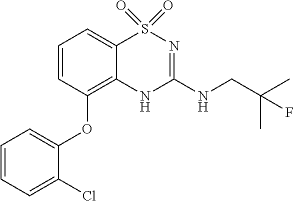 [SMILES: CC(C)(F)CNC1=NS(=O)(=O)c2cccc(Oc3ccccc3Cl)c2N1] | 5-(2-chlorophenoxy)-3-((2-fluoro- 2-methylpropyl)amino)-4H- benzo[e][1,2,4]thiadiazine 1,1- dioxide |
| 1-245 | 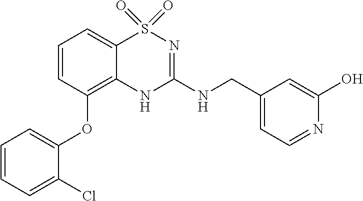 [SMILES: O=S1(=O)N=C(NCc2ccnc(O)c2)Nc2c(Oc3ccccc3Cl)cccc21] | 5-(2-chlorophenoxy)-3-(((2- hydroxypyridin-4- yl)methyl)amino)-4H- benzo[e][1,2,4]thiadiazine 1,1- dioxide |
| 1-246 | 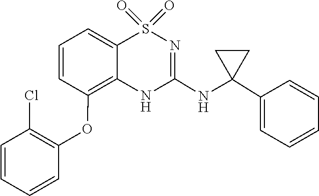 [SMILES: O=S1(=O)N=C(NC2(c3ccccc3)CC2)Nc2c(Oc3ccccc3Cl)cccc21] | 5-(2-chlorophenoxy)-3-((1- phenylcyclopropyl)amino)-4H- benzo[e][1,2,4]thiadiazine 1,1- dioxide |
| 1-247 | 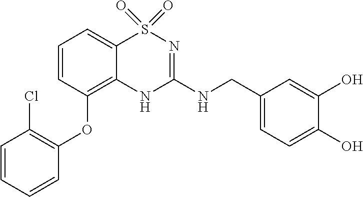 [SMILES: O=S1(=O)N=C(NCc2ccc(O)c(O)c2)Nc2c(Oc3ccccc3Cl)cccc21] | 5-(2-chlorophenoxy)-3-((3,4- dihydroxybenzyl)amino)-4H- benzo[e][1,2,4]thiadiazine 1,1- dioxide |
| 1-248 | 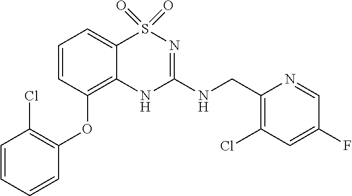 [SMILES: O=S1(=O)N=C(NCc2ncc(F)cc2Cl)Nc2c(Oc3ccccc3Cl)cccc21] | 3-(((3-chloro-5-fluoropyridin-2- yl)methyl)amino)-5-(2- chlorophenoxy)-4H- benzo[e][1,2,4]thiadiazine 1,1- dioxide |
| 1-249 | 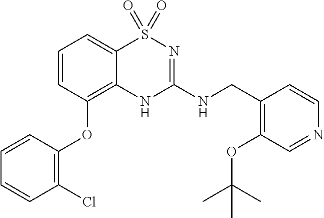 [SMILES: CC(C)(C)Oc1cnccc1CNC1=NS(=O)(=O)c2cccc(Oc3ccccc3Cl)c2N1] | 3-(((3-(tert-butoxy)pyridin-4- yl)methyl)amino)-5-(2- chlorophenoxy)-4H- benzo[e][1,2,4]thiadiazine 1,1- dioxide |
| 1-250 | 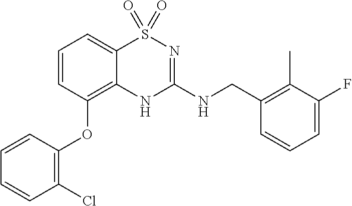 [SMILES: Cc1c(F)cccc1CNC1=NS(=O)(=O)c2cccc(Oc3ccccc3Cl)c2N1] | 5-(2-chlorophenoxy)-3-((3-fluoro- 2-methylbenzyl)amino)-4H- benzo[e][1,2,4]thiadiazine 1,1- dioxide |
| 1-251 | 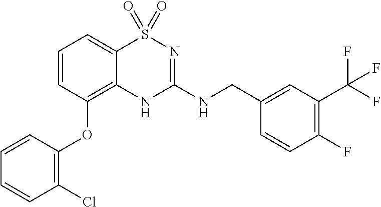 [SMILES: O=S1(=O)N=C(NCc2ccc(F)c(C(F)(F)F)c2)Nc2c(Oc3ccccc3Cl)cccc21] | 5-(2-chlorophenoxy)-3-((4-fluoro- 3-(trifluoromethyl)benzyl)amino)- 4H-benzo[e][1,2,4]thiadiazine 1,1- dioxide |
| 1-252 | 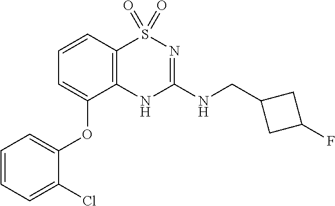 [SMILES: O=S1(=O)N=C(NCC2CC(F)C2)Nc2c(Oc3ccccc3Cl)cccc21] | 5-(2-chlorophenoxy)-3-(((3- fluorocyclobutyl)methyl)amino)- 4H-benzo[e][1,2,4]thiadiazine 1,1- dioxide |
| 1-253 | 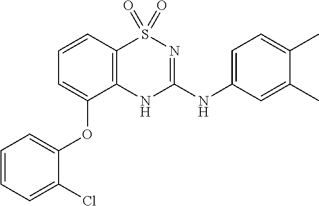 [SMILES: Cc1ccc(NC2=NS(=O)(=O)c3cccc(Oc4ccccc4Cl)c3N2)cc1C] | 5-(2-chlorophenoxy)-3-((3,4- dimethylphenyl)amino)-4H- benzo[e][1,2,4]thiadiazine 1,1- dioxide |
| 1-254 | 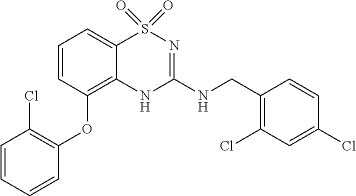 [SMILES: O=S1(=O)N=C(NCc2ccc(Cl)cc2Cl)Nc2c(Oc3ccccc3Cl)cccc21] | 5-(2-chlorophenoxy)-3-((2,4- dichlorobenzyl)amino)-4H- benzo[e][1,2,4]thiadiazine 1,1- dioxide |
| 1-255 | 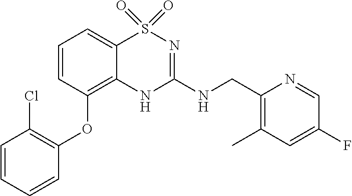 [SMILES: Cc1cc(F)cnc1CNC1=NS(=O)(=O)c2cccc(Oc3ccccc3Cl)c2N1] | 5-(2-chlorophenoxy)-3-(((5-fluoro- 3-methylpyridin-2- yl)methyl)amino)-4H- benzo[e][1,2,4]thiadiazine 1,1- dioxide |
| 1-256 | 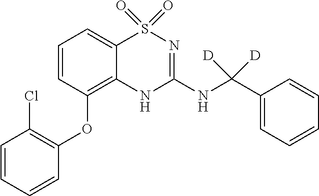 [SMILES: [2H]C([2H])(NC1=NS(=O)(=O)c2cccc(Oc3ccccc3Cl)c2N1)c1ccccc1] | 5-(2-chlorophenoxy)-3- ((phenylmethyl-d2)amino)-4H- benzo[e][1,2,4]thiadiazine 1,1- dioxide |
| 1-257 | 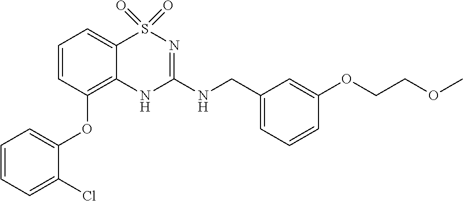 [SMILES: COCCOc1cccc(CNC2=NS(=O)(=O)c3cccc(Oc4ccccc4Cl)c3N2)c1] | 5-(2-chlorophenoxy)-3-((3-(2- methoxyethoxy)benzyl)amino)- 4H-benzo[e][1,2,4]thiadiazine 1,1- dioxide |
| 1-259 (racemic) | 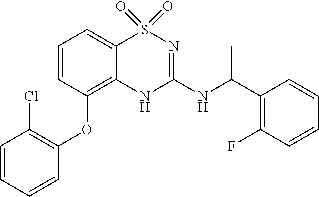 [SMILES: CC(NC1=NS(=O)(=O)c2cccc(Oc3ccccc3Cl)c2N1)c1ccccc1F] | 5-(2-chlorophenoxy)-3-((1-(2- fluorophenyl)ethyl)amino)-4H- benzo[e][1,2,4]thiadiazine 1,1- dioxide |
| 1-259 | 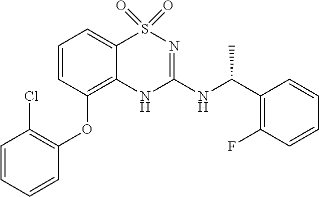 [SMILES: C[C@@H](NC1=NS(=O)(=O)c2cccc(Oc3ccccc3Cl)c2N1)c1ccccc1F] | (R)-5-(2-chlorophenoxy)-3-((1-(2- fluorophenyl)ethyl)amino)-4H- benzo[e][1,2,4]thiadiazine 1,1- dioxide |
| 1-260 | 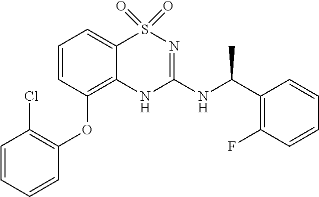 [SMILES: C[C@H](NC1=NS(=O)(=O)c2cccc(Oc3ccccc3Cl)c2N1)c1ccccc1F] | (S)-5-(2-chlorophenoxy)-3-((1-(2- fluorophenyl)ethyl)amino)-4H- benzo[e][1,2,4]thiadiazine 1,1- dioxide |
| 1-261 | 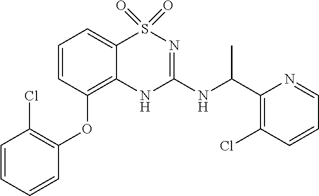 [SMILES: CC(NC1=NS(=O)(=O)c2cccc(Oc3ccccc3Cl)c2N1)c1ncccc1Cl] | 5-(2-chlorophenoxy)-3-((1-(3- chloropyridin-2-yl)ethyl)amino)- 4H-benzo[e][1,2,4]thiadiazine 1,1- dioxide |
| 1-261 (R) | 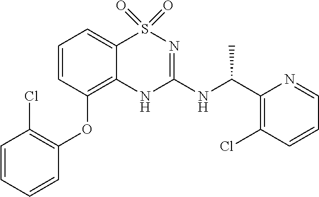 [SMILES: C[C@@H](NC1=NS(=O)(=O)c2cccc(Oc3ccccc3Cl)c2N1)c1ncccc1Cl] | (R)-5-(2-chlorophenoxy)-3-((1-(3- chloropyridin-2-yl)ethyl)amino)- 4H-benzo[e][1,2,4]thiadiazine 1,1- dioxide |
| 1-261 (S) | 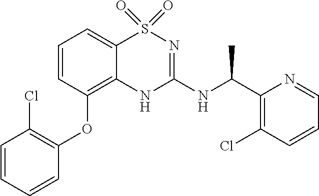 [SMILES: C[C@H](NC1=NS(=O)(=O)c2cccc(Oc3ccccc3Cl)c2N1)c1ncccc1Cl] | (S)-5-(2-chlorophenoxy)-3-((1-(3- chloropyridin-2-yl)ethyl)amino)- 4H-benzo[e][1,2,4]thiadiazine 1,1- dioxide |
| 1-262 | 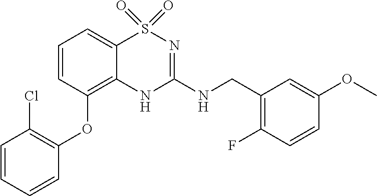 [SMILES: COc1ccc(F)c(CNC2=NS(=O)(=O)c3cccc(Oc4ccccc4Cl)c3N2)c1] | 5-(2-chlorophenoxy)-3-((2-fluoro- 5-methoxybenzyl)amino)-4H- benzo[e][1,2,4]thiadiazine 1,1- dioxide |
| 1-263 | 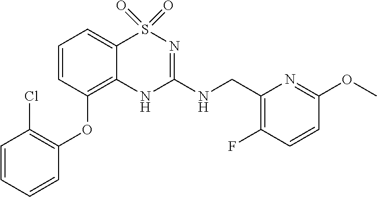 [SMILES: COc1ccc(F)c(CNC2=NS(=O)(=O)c3cccc(Oc4ccccc4Cl)c3N2)n1] | 5-(2-chlorophenoxy)-3-(((3-fluoro- 6-methoxypyridin-2- yl)methyl)amino)-4H- benzo[e][1,2,4]thiadiazine 1,1- dioxide |
| 1-264 | 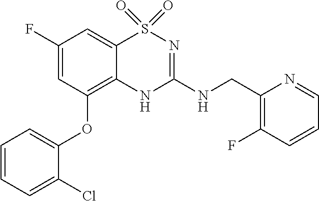 [SMILES: O=S1(=O)N=C(NCc2ncccc2F)Nc2c(Oc3ccccc3Cl)cc(F)cc21] | 5-(2-chlorophenoxy)-7-fluoro-3- (((3-fluoropyridin-2- yl)methyl)amino)-4H- benzo[e][1,2,4]thiadiazine 1,1- dioxide |
| 1-265 | 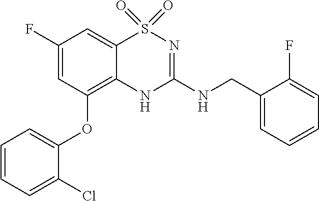 [SMILES: O=S1(=O)N=C(NCc2ccccc2F)Nc2c(Oc3ccccc3Cl)cc(F)cc21] | 5-(2-chlorophenoxy)-7-fluoro-3- ((2-fluorobenzyl)amino)-4H- benzo[e][1,2,4]thiadiazine 1,1- dioxide |
| 1-266 | 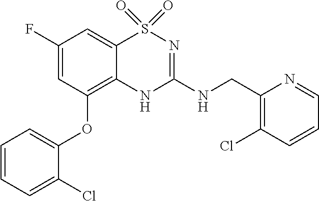 [SMILES: O=S1(=O)N=C(NCc2ncccc2Cl)Nc2c(Oc3ccccc3Cl)cc(F)cc21] | 5-(2-chlorophenoxy)-3-(((3- chloropyridin-2-yl)methyl)amino)- 7-fluoro-4H- benzo[e][1,2,4]thiadiazine 1,1- dioxide |
| 1-267 | 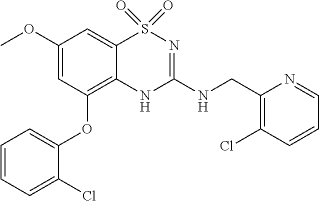 [SMILES: COc1cc(Oc2ccccc2Cl)c2c(c1)S(=O)(=O)N=C(NCc1ncccc1Cl)N2] | 5-(2-chlorophenoxy)-3-(((3- chloropyridin-2-yl)methyl)amino)- 7-methoxy-4H- benzo[e][1,2,4]thiadiazine 1,1- dioxide |
| 1-268 | 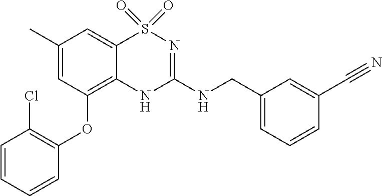 [SMILES: Cc1cc(Oc2ccccc2Cl)c2c(c1)S(=O)(=O)N=C(NCc1cccc(C#N)c1)N2] | 3-(((5-(2-chlorophenoxy)-7- methyl-1,1-dioxido-4H- benzo[e][1,2,4]thiadiazin-3- yl)amino)methyl)benzonitrile |
| 1-269 | 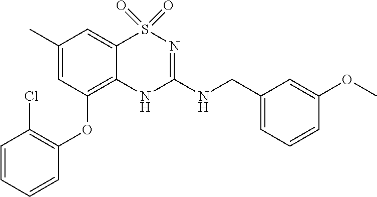 [SMILES: COc1cccc(CNC2=NS(=O)(=O)c3cc(C)cc(Oc4ccccc4Cl)c3N2)c1] | 5-(2-chlorophenoxy)-3-((3- methoxybenzyl)amino)-7-methyl- 4H-benzo[e][1,2,4]thiadiazine 1,1- dioxide |
| 1-270 | 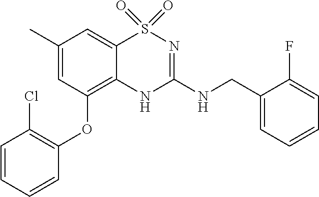 [SMILES: Cc1cc(Oc2ccccc2Cl)c2c(c1)S(=O)(=O)N=C(NCc1ccccc1F)N2] | 5-(2-chlorophenoxy)-3-((2- fluorobenzyl)amino)-7-methyl-4H- benzo[e][1,2,4]thiadiazine 1,1- dioxide |
| 1-271 | 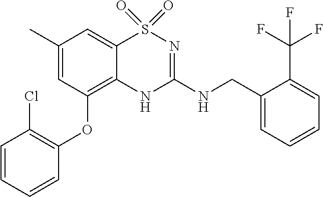 [SMILES: Cc1cc(Oc2ccccc2Cl)c2c(c1)S(=O)(=O)N=C(NCc1ccccc1C(F)(F)F)N2] | 5-(2-chlorophenoxy)-7-methyl-3- ((2- (trifluoromethyl)benzyl)amino)- 4H-benzo[e][1,2,4]thiadiazine 1,1- dioxide |
| 1-272 | 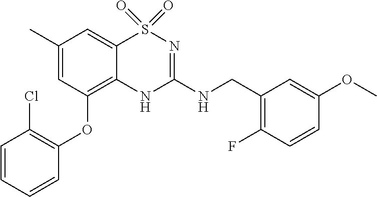 [SMILES: COc1ccc(F)c(CNC2=NS(=O)(=O)c3cc(C)cc(Oc4ccccc4Cl)c3N2)c1] | 5-(2-chlorophenoxy)-3-((2-fluoro- 5-methoxybenzyl)amino)-7- methyl-4H- benzo[e][1,2,4]thiadiazine 1,1- dioxide |
| 1-273 | 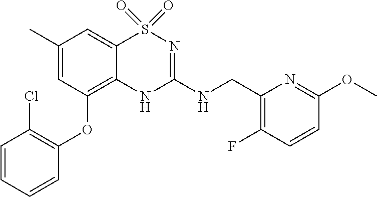 [SMILES: COc1ccc(F)c(CNC2=NS(=O)(=O)c3cc(C)cc(Oc4ccccc4Cl)c3N2)n1] | 5-(2-chlorophenoxy)-3-(((3-fluoro- 6-methoxypyridin-2- yl)methyl)amino)-7-methyl-4H- benzo[e][1,2,4]thiadiazine 1,1- dioxide |
| 1-274 | 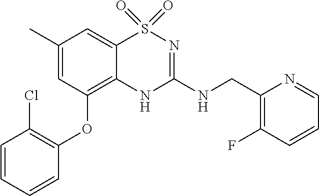 [SMILES: Cc1cc(Oc2ccccc2Cl)c2c(c1)S(=O)(=O)N=C(NCc1ncccc1F)N2] | 5-(2-chlorophenoxy)-3-(((3- fluoropyridin-2-yl)methyl)amino)- 7-methyl-4H- benzo[e][1,2,4]thiadiazine 1,1- dioxide |
| 1-275 | 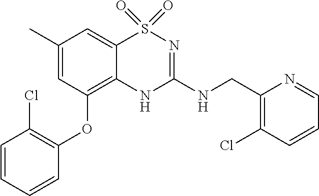 [SMILES: Cc1cc(Oc2ccccc2Cl)c2c(c1)S(=O)(=O)N=C(NCc1ncccc1Cl)N2] | 5-(2-chlorophenoxy)-3-(((3- chloropyridin-2-yl)methyl)amino)- 7-methyl-4H- benzo[e][1,2,4]thiadiazine 1,1- dioxide |
| 1-276 (racemic) | 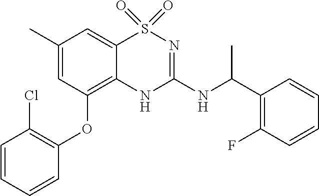 [SMILES: Cc1cc(Oc2ccccc2Cl)c2c(c1)S(=O)(=O)N=C(NC(C)c1ccccc1F)N2] | 5-(2-chlorophenoxy)-3-((1-(2- fluorophenyl)ethyl)amino)-7- methyl-4H- benzo[e][1,2,4]thiadiazine 1,1- dioxide |
| 1-276 | 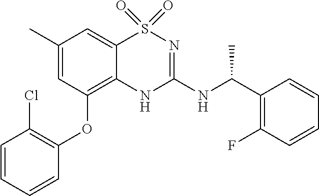 [SMILES: Cc1cc(Oc2ccccc2Cl)c2c(c1)S(=O)(=O)N=C(N[C@H](C)c1ccccc1F)N2] | (R)-5-(2-chlorophenoxy)-3-((1-(2- fluorophenyl)ethyl)amino)-7- methyl-4H- benzo[e][1,2,4]thiadiazine 1,1- dioxide |
| 1-276 (S) | 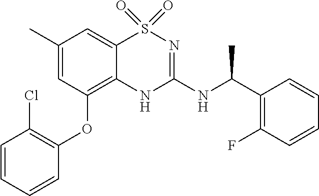 [SMILES: Cc1cc(Oc2ccccc2Cl)c2c(c1)S(=O)(=O)N=C(N[C@@H](C)c1ccccc1F)N2] | (S)-5-(2-chlorophenoxy)-3-((1-(2- fluorophenyl)ethyl)amino)-7- methyl-4H- benzo[e][1,2,4]thiadiazine 1,1- dioxide |
| 1-277 (racemic) | 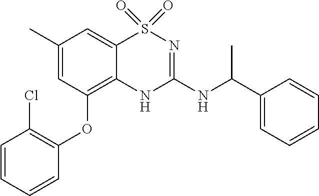 [SMILES: Cc1cc(Oc2ccccc2Cl)c2c(c1)S(=O)(=O)N=C(NC(C)c1ccccc1)N2] | 5-(2-chlorophenoxy)-7-methyl-3- ((1-phenylethyl)amino)-4H- benzo[e][1,2,4]thiadiazine 1,1- dioxide |
| 1-277 | 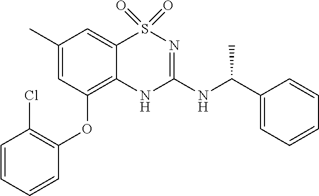 [SMILES: Cc1cc(Oc2ccccc2Cl)c2c(c1)S(=O)(=O)N=C(N[C@H](C)c1ccccc1)N2] | (R)-5-(2-chlorophenoxy)-7-methyl- 3-((1-phenylethyl)amino)-4H- benzo[e][1,2,4]thiadiazine 1,1- dioxide |
| 1-277 (S) | 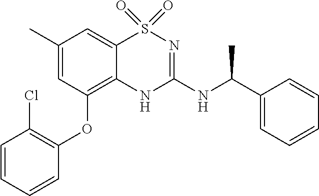 [SMILES: Cc1cc(Oc2ccccc2Cl)c2c(c1)S(=O)(=O)N=C(N[C@@H](C)c1ccccc1)N2] | (S)-5-(2-chlorophenoxy)-7-methyl- 3-((1-phenylethyl)amino)-4H- benzo[e][1,2,4]thiadiazine 1,1- dioxide |
| 1-279 | 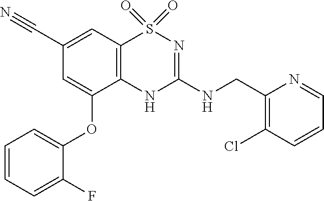 [SMILES: N#Cc1cc(Oc2ccccc2F)c2c(c1)S(=O)(=O)N=C(NCc1ncccc1Cl)N2] | 3-(((3-chloropyridin-2- yl)methyl)amino)-5-(2- fluorophenoxy)-4H- benzo[e][1,2,4]thiadiazine-7- carbonitrile 1,1-dioxide |
| 1-280 | 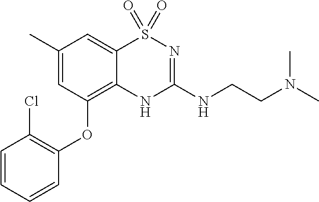 [SMILES: Cc1cc(Oc2ccccc2Cl)c2c(c1)S(=O)(=O)N=C(NCCN(C)C)N2] | 7-methyl-5-(2-chlorophenoxy)-3- (((2-dimethylamino)ethyl)amino)- 4H-benzo[e][1,2,4]thiadiazine 1,1- dioxide |
| 2-1 | 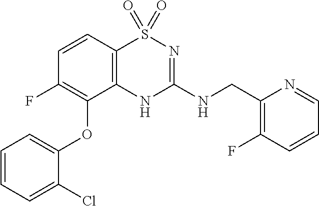 [SMILES: O=S1(=O)N=C(NCc2ncccc2F)Nc2c1ccc(F)c2Oc1ccccc1Cl] | 5-(2-chlorophenoxy)-6-fluoro-3- (((3-fluoropyridin-2- yl)methyl)amino)-4H- benzo[e][1,2,4]thiadiazine 1,1- dioxide |
| 2-2 | 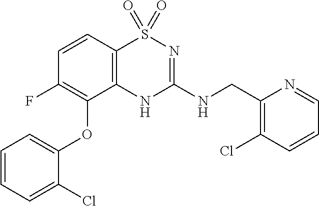 [SMILES: O=S1(=O)N=C(NCc2ncccc2Cl)Nc2c1ccc(F)c2Oc1ccccc1Cl] | 5-(2-chlorophenoxy)-3-(((3- chloropyridin-2-yl)methyl)amino)- 6-fluoro-4H- benzo[e][1,2,4]thiadiazine 1,1- dioxide |
| 2-3 (racemic) | 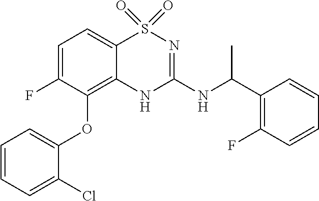 [SMILES: CC(NC1=NS(=O)(=O)c2ccc(F)c(Oc3ccccc3Cl)c2N1)c1ccccc1F] | 5-(2-chlorophenoxy)-6-fluoro-3- ((1-(2-fluorophenyl)ethyl)amino)- 4H-benzo[e][1,2,4]thiadiazine 1,1- dioxide |
| 2-3 | 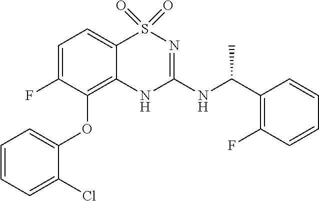 [SMILES: C[C@@H](NC1=NS(=O)(=O)c2ccc(F)c(Oc3ccccc3Cl)c2N1)c1ccccc1F] | (R)-5-(2-chlorophenoxy)-6-fluoro- 3-((1-(2- fluorophenyl)ethyl)amino)-4H- benzo[e][1,2,4]thiadiazine 1,1- dioxide |
| 2-3 (S) | 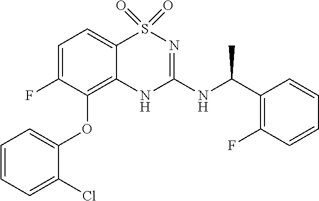 [SMILES: C[C@H](NC1=NS(=O)(=O)c2ccc(F)c(Oc3ccccc3Cl)c2N1)c1ccccc1F] | (S)-5-(2-chlorophenoxy)-6-fluoro- 3-((1-(2- fluorophenyl)ethyl)amino)-4H- benzo[e][1,2,4]thiadiazine 1,1- dioxide |
| 2-4 | 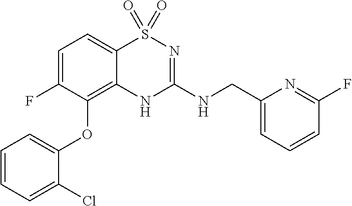 [SMILES: O=S1(=O)N=C(NCc2cccc(F)n2)Nc2c1ccc(F)c2Oc1ccccc1Cl] | 5-(2-chlorophenoxy)-6-fluoro-3- (((6-fluoropyridin-2- yl)methyl)amino)-4H- benzo[e][1,2,4]thiadiazine 1,1- dioxide |
| 2-5 | 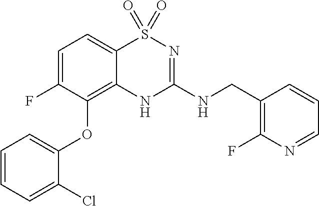 [SMILES: O=S1(=O)N=C(NCc2cccnc2F)Nc2c1ccc(F)c2Oc1ccccc1Cl] | 5-(2-chlorophenoxy)-6-fluoro-3- (((2-fluoropyridin-3- yl)methyl)amino)-4H- benzo[e][1,2,4]thiadiazine 1,1- dioxide |
| 2-6 | 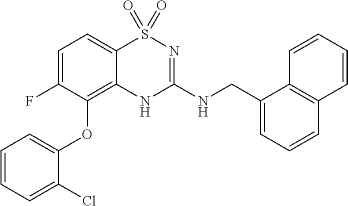 [SMILES: O=S1(=O)N=C(NCc2cccc3ccccc23)Nc2c1ccc(F)c2Oc1ccccc1Cl] | 5-(2-chlorophenoxy)-6-fluoro-3- ((naphthalen-1-ylmethyl)amino)- 4H-benzo[e][1,2,4]thiadiazine 1,1- dioxide |
| 2-7 | 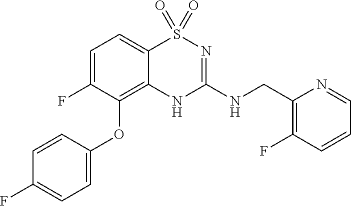 [SMILES: O=S1(=O)N=C(NCc2ncccc2F)Nc2c1ccc(F)c2Oc1ccc(F)cc1] | 6-fluoro-5-(4-fluorophenoxy)-3- (((3-fluoropyridin-2- yl)methyl)amino)-4H- benzo[e][1,2,4]thiadiazine 1,1- dioxide |
| 2-8 | 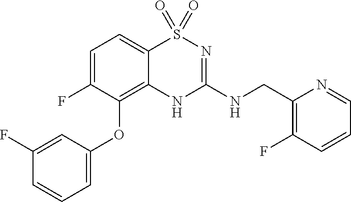 [SMILES: O=S1(=O)N=C(NCc2ncccc2F)Nc2c1ccc(F)c2Oc1cccc(F)c1] | 6-fluoro-5-(3-fluorophenoxy)-3- (((3-fluoropyridin-2- yl)methyl)amino)-4H- benzo[e][1,2,4]thiadiazine 1,1- dioxide |
| 2-9 | 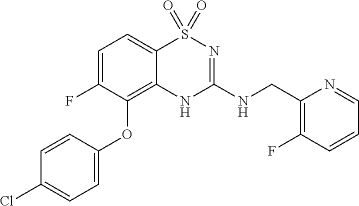 [SMILES: O=S1(=O)N=C(NCc2ncccc2F)Nc2c1ccc(F)c2Oc1ccc(Cl)cc1] | 5-(4-chlorophenoxy)-6-fluoro-3- (((3-fluoropyridin-2- yl)methyl)amino)-4H- benzo[e][1,2,4]thiadiazine 1,1- dioxide |
| 2-10 | 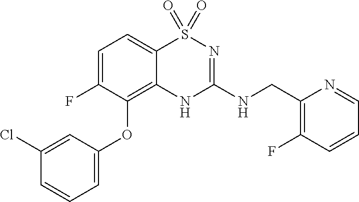 [SMILES: O=S1(=O)N=C(NCc2ncccc2F)Nc2c1ccc(F)c2Oc1cccc(Cl)c1] | 5-(3-chlorophenoxy)-6-fluoro-3- (((3-fluoropyridin-2- yl)methyl)amino)-4H- benzo[e][1,2,4]thiadiazine 1,1- dioxide |
| 2-11 | 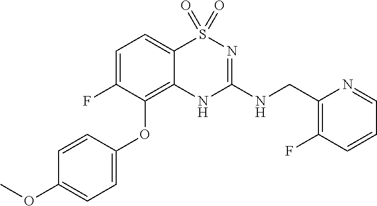 [SMILES: COc1ccc(Oc2c(F)ccc3c2NC(NCc2ncccc2F)=NS3(=O)=O)cc1] | 6-fluoro-3-(((3-fluoropyridin-2- yl)methyl)amino)-5-(4- methoxyphenoxy)-4H- benzo[e][1,2,4]thiadiazine 1,1- dioxide |
| 2-12 | 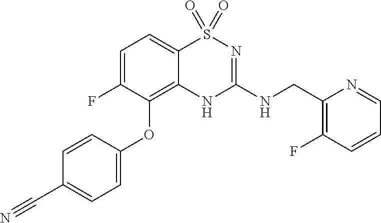 [SMILES: N#Cc1ccc(Oc2c(F)ccc3c2NC(NCc2ncccc2F)=NS3(=O)=O)cc1] | 4-((6-fluoro-3-(((3-fluoropyridin- 2-yl)methyl)amino)-1,1-dioxido- 4H-benzo[e][1,2,4]thiadiazin-5- yl)oxy)benzonitrile |
| 2-13 | 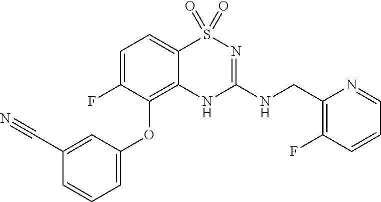 [SMILES: N#Cc1cccc(Oc2c(F)ccc3c2NC(NCc2ncccc2F)=NS3(=O)=O)c1] | 3-((6-fluoro-3-(((3-fluoropyridin- 2-yl)methyl)amino)-1,1-dioxido- 4H-benzo[e][1,2,4]thiadiazin-5- yl)oxy)benzonitrile |
| 2-14 | 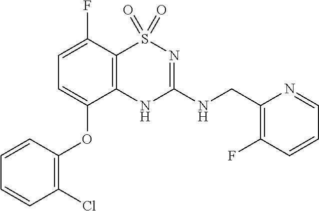 [SMILES: O=S1(=O)N=C(NCc2ncccc2F)Nc2c(Oc3ccccc3Cl)ccc(F)c21] | 3-(((3-fluoropyridin-2- yl)methyl)amino)-5-(2- chlorophenoxy)-8-fluoro-4H- benzo[e][1,2,4]thiadiazine-6- carbonitrile 1,1-dioxide |
| 2-15 | 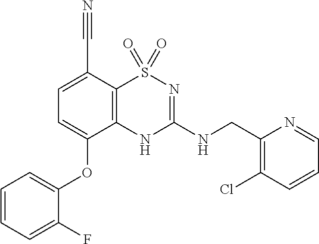 [SMILES: N#Cc1ccc(Oc2ccccc2F)c2c1S(=O)(=O)N=C(NCc1ncccc1Cl)N2] | 3-(((3-chloropyridin-2- yl)methyl)amino)-5-(2- fluorophenoxy)-8-cyano-4H- benzo[e][1,2,4]thiadiazine-6- carbonitrile 1,1-dioxide |
| 3-1 | 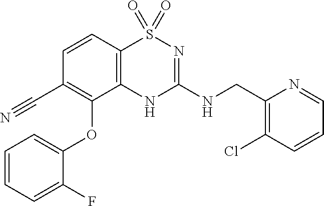 [SMILES: N#Cc1ccc2c(c1Oc1ccccc1F)NC(NCc1ncccc1Cl)=NS2(=O)=O] | 3-(((3-chloropyridin-2- yl)methyl)amino)-5-(2- fluorophenoxy)-4H- benzo[e][1,2,4]thiadiazine-6- carbonitrile 1,1-dioxide |
| 3-2 | 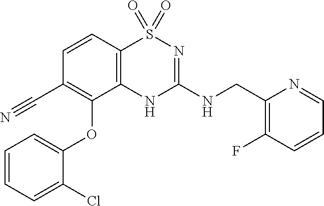 [SMILES: N#Cc1ccc2c(c1Oc1ccccc1Cl)NC(NCc1ncccc1F)=NS2(=O)=O] | 5-(2-chlorophenoxy)-3-(((3- fluoropyridin-2-yl)methyl)amino)- 4H-benzo[e][1,2,4]thiadiazine-6- carbonitrile 1,1-dioxide |
| 4-1 | 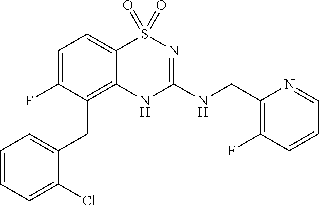 [SMILES: O=S1(=O)N=C(NCc2ncccc2F)Nc2c1ccc(F)c2Cc1ccccc1Cl] | 5-(2-chlorobenzyl)-6-fluoro-3-(((3- fluoropyridin-2-yl)methyl)amino)- 4H-benzo[e][1,2,4]thiadiazine 1,1- dioxide |
| 4-2 | 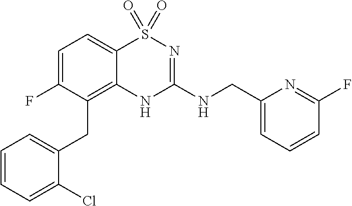 [SMILES: O=S1(=O)N=C(NCc2cccc(F)n2)Nc2c1ccc(F)c2Cc1ccccc1Cl] | 5-(2-chlorobenzyl)-6-fluoro-3-(((6- fluoropyridin-2-yl)methyl)amino)- 4H-benzo[e][1,2,4]thiadiazine 1,1- dioxide |
| 4-3 | 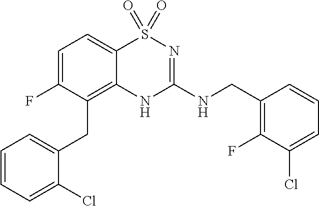 [SMILES: O=S1(=O)N=C(NCc2cccc(Cl)c2F)Nc2c1ccc(F)c2Cc1ccccc1Cl] | 3-((3-chloro-2- fluorobenzyl)amino)-5-(2- chlorobenzyl)-6-fluoro-4H- benzo[e][1,2,4]thiadiazine 1,1- dioxide |
| 4-4 | 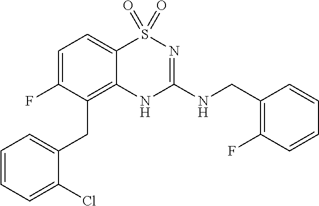 [SMILES: O=S1(=O)N=C(NCc2ccccc2F)Nc2c1ccc(F)c2Cc1ccccc1Cl] | 5-(2-chlorobenzyl)-6-fluoro-3-((2- fluorobenzyl)amino)-4H- benzo[e][1,2,4]thiadiazine 1,1- dioxide |
| 4-5 | 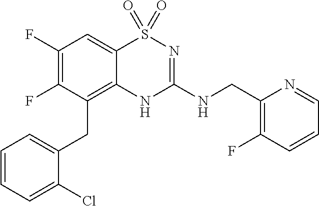 [SMILES: O=S1(=O)N=C(NCc2ncccc2F)Nc2c1cc(F)c(F)c2Cc1ccccc1Cl] | 5-(2-chlorobenzyl)-6,7-difluoro-3- (((3-fluoropyridin-2- yl)methyl)amino)-4H- benzo[e][1,2,4]thiadiazine 1,1- dioxide |
| 4-6 | 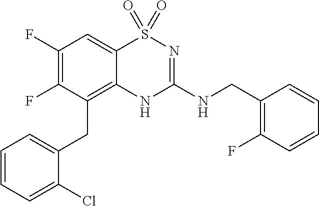 [SMILES: O=S1(=O)N=C(NCc2ccccc2F)Nc2c1cc(F)c(F)c2Cc1ccccc1Cl] | 5-(2-chlorobenzyl)-6,7-difluoro-3- ((2-fluorobenzyl)amino)-4H- benzo[e][1,2,4]thiadiazine 1,1- dioxide |
| 4-7 | 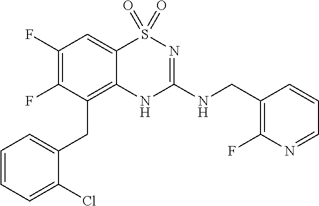 [SMILES: O=S1(=O)N=C(NCc2cccnc2F)Nc2c1cc(F)c(F)c2Cc1ccccc1Cl] | 5-(2-chlorobenzyl)-6,7-difluoro-3- (((2-fluoropyridin-3- yl)methyl)amino)-4H- benzo[e][1,2,4]thiadiazine 1,1- dioxide |
| 4-8 | 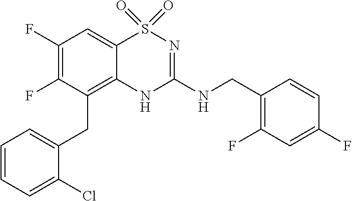 [SMILES: O=S1(=O)N=C(NCc2ccc(F)cc2F)Nc2c1cc(F)c(F)c2Cc1ccccc1Cl] | 5-(2-chlorobenzyl)-3-((2,4- difluorobenzyl)amino)-6,7- difluoro-4H- benzo[e][1,2,4]thiadiazine 1,1- dioxide |
| 4-9 | 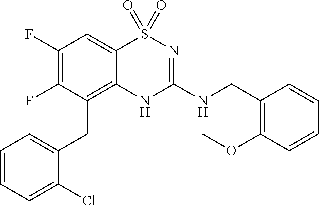 [SMILES: COc1ccccc1CNC1=NS(=O)(=O)c2cc(F)c(F)c(Cc3ccccc3Cl)c2N1] | 5-(2-chlorobenzyl)-6,7-difluoro-3- ((2-methoxybenzyl)amino)-4H- benzo[e][1,2,4]thiadiazine 1,1- dioxide |
| 4-10 | 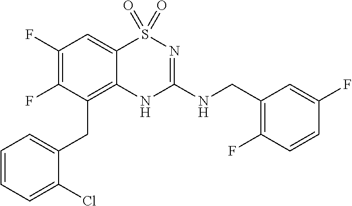 [SMILES: O=S1(=O)N=C(NCc2cc(F)ccc2F)Nc2c1cc(F)c(F)c2Cc1ccccc1Cl] | 5-(2-chlorobenzyl)-3-((2,5- difluorobenzyl)amino)-6,7- difluoro-4H- benzo[e][1,2,4]thiadiazine 1,1- dioxide |
| 4-11 | 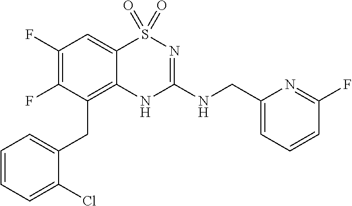 [SMILES: O=S1(=O)N=C(NCc2cccc(F)n2)Nc2c1cc(F)c(F)c2Cc1ccccc1Cl] | 5-(2-chlorobenzyl)-6,7-difluoro-3- (((6-fluoropyridin-2- yl)methyl)amino)-4H- benzo[e][1,2,4]thiadiazine 1,1- dioxide |
| 4-12 | 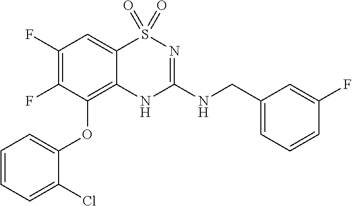 [SMILES: O=S1(=O)N=C(NCc2cccc(F)c2)Nc2c1cc(F)c(F)c2Oc1ccccc1Cl] | 5-(2-chlorobenzyl)-6,7-difluoro-3- ((3-fluorobenzyl)amino)-4H- benzo[e][1,2,4]thiadiazine 1,1- dioxide |
| 4-13 | 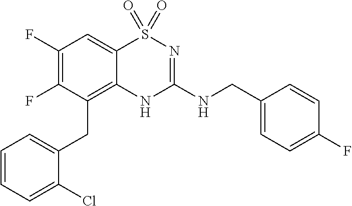 [SMILES: O=S1(=O)N=C(NCc2ccc(F)cc2)Nc2c1cc(F)c(F)c2Cc1ccccc1Cl] | 5-(2-chlorobenzyl)-6,7-difluoro-3- ((4-fluorobenzyl)amino)-4H- benzo[e][1,2,4]thiadiazine 1,1- dioxide |
| 4-14 | 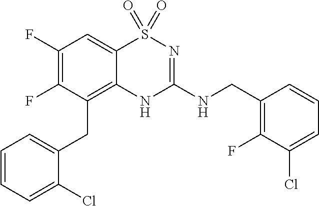 [SMILES: O=S1(=O)N=C(NCc2cccc(Cl)c2F)Nc2c1cc(F)c(F)c2Cc1ccccc1Cl] | 3-((3-chloro-2- fluorobenzyl)amino)-5-(2- chlorobenzyl)-6,7-difluoro-4H- benzo[e][1,2,4]thiadiazine 1,1- dioxide |
| 4-15 | 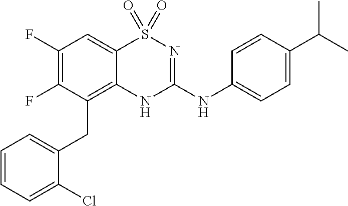 [SMILES: CC(C)c1ccc(NC2=NS(=O)(=O)c3cc(F)c(F)c(Cc4ccccc4Cl)c3N2)cc1] | 5-(2-chlorobenzyl)-6,7-difluoro-3- ((4-isopropylphenyl)amino)-4H- benzo[e][1,2,4]thiadiazine 1,1- dioxide |
| 4-16 | 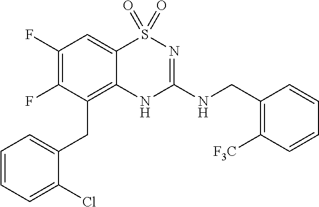 [SMILES: O=S1(=O)N=C(NCc2ccccc2C(F)(F)F)Nc2c1cc(F)c(F)c2Cc1ccccc1Cl] | 5-(2-chlorobenzyl)-6,7-difluoro-3- ((2- (trifluoromethyl)benzyl)amino)- 4H-benzo[e][1,2,4]thiadiazine 1,1- dioxide |
| 4-17 | 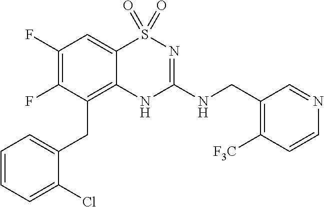 [SMILES: O=S1(=O)N=C(NCc2cnccc2C(F)(F)F)Nc2c1cc(F)c(F)c2Cc1ccccc1Cl] | 5-(2-chlorobenzyl)-6,7-difluoro-3- (((4-(trifluoromethyl)pyridin-3- yl)methyl)amino)-4H- benzo[e][1,2,4]thiadiazine 1,1- dioxide |
| 4-18 | 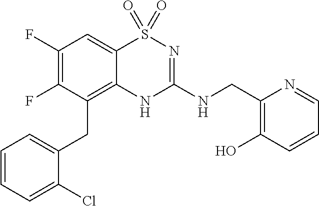 [SMILES: O=S1(=O)N=C(NCc2ncccc2O)Nc2c1cc(F)c(F)c2Cc1ccccc1Cl] | 5-(2-chlorobenzyl)-6,7-difluoro-3- (((3-hydroxypyridin-2- yl)methyl)amino)-4H- benzo[e][1,2,4]thiadiazine 1,1- dioxide |
| 4-19 | 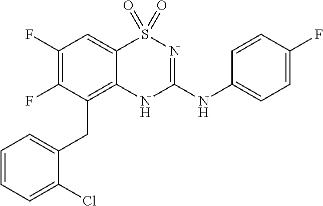 [SMILES: O=S1(=O)N=C(Nc2ccc(F)cc2)Nc2c1cc(F)c(F)c2Cc1ccccc1Cl] | 5-(2-chlorobenzyl)-6,7-difluoro-3- ((4-fluorophenyl)amino)-4H- benzo[e][1,2,4]thiadiazine 1,1- dioxide |
| 4-20 | 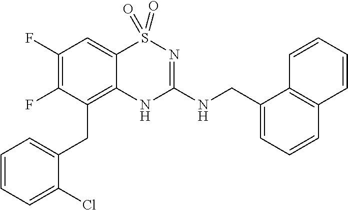 [SMILES: O=S1(=O)N=C(NCc2cccc3ccccc23)Nc2c1cc(F)c(F)c2Cc1ccccc1Cl] | 5-(2-chlorobenzyl)-6,7-difluoro-3- ((naphthalen-1-ylmethyl)amino)- 4H-benzo[e][1,2,4]thiadiazine 1,1- dioxide |
| 5-1 | 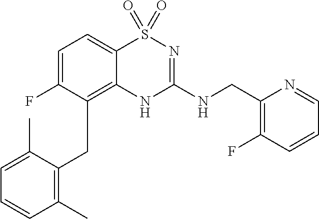 [SMILES: Cc1cccc(C)c1Cc1c(F)ccc2c1NC(NCc1ncccc1F)=NS2(=O)=O] | 5-(2,6-dimethylbenzyl)-6-fluoro-3- (((3-fluoropyridin-2- yl)methyl)amino)-4H- benzo[e][1,2,4]thiadiazine 1,1- dioxide |
| 6-1 | 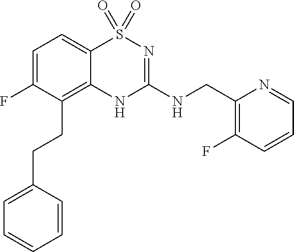 [SMILES: O=S1(=O)N=C(NCc2ncccc2F)Nc2c1ccc(F)c2CCc1ccccc1] | 6-fluoro-3-(((3-fluoropyridin-2- yl)methyl)amino)-5-phenethyl-4H- benzo[e][1,2,4]thiadiazine 1,1- dioxide |
| 6-2 | 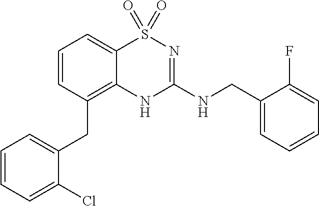 [SMILES: O=S1(=O)N=C(NCc2ccccc2F)Nc2c(Cc3ccccc3Cl)cccc21] | 5-(2-chlorobenzyl)-3-((2- fluorobenzyl)amino)-4H- benzo[e][1,2,4]thiadiazine 1,1- dioxide |
| 6-3 | 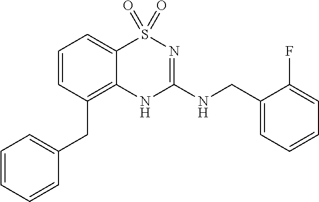 [SMILES: O=S1(=O)N=C(NCc2ccccc2F)Nc2c(Cc3ccccc3)cccc21] | 5-benzyl-3-((2- fluorobenzyl)amino)-4H- benzo[e][1,2,4]thiadiazine 1,1- dioxide |
| 6-4 | 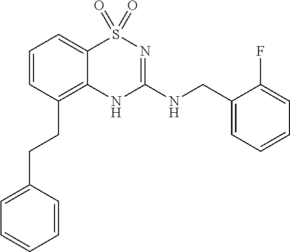 [SMILES: O=S1(=O)N=C(NCc2ccccc2F)Nc2c(CCc3ccccc3)cccc21] | 3-((2-fluorobenzyl)amino)-5- phenethyl-4H- benzo[e][1,2,4]thiadiazine 1,1- dioxide |
| 6-5 | 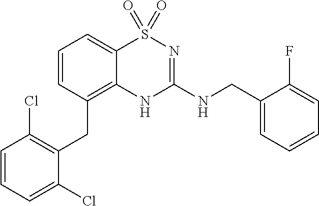 [SMILES: O=S1(=O)N=C(NCc2ccccc2F)Nc2c(Cc3c(Cl)cccc3Cl)cccc21] | 5-(2,6-dichlorobenzyl)-3-((2- fluorobenzyl)amino)-4H- benzo[e][1,2,4]thiadiazine 1,1- dioxide |
| 6-6 | 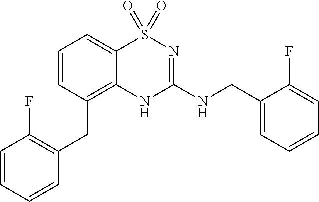 [SMILES: O=S1(=O)N=C(NCc2ccccc2F)Nc2c(Cc3ccccc3F)cccc21] | 5-(2-fluorobenzyl)-3-((2- fluorobenzyl)amino)-4H- benzo[e][1,2,4]thiadiazine 1,1- dioxide |
| 6-7 | 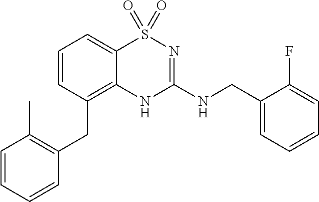 [SMILES: Cc1ccccc1Cc1cccc2c1NC(NCc1ccccc1F)=NS2(=O)=O] | 3-((2-fluorobenzyl)amino)-5-(2- methylbenzyl)-4H- benzo[e][1,2,4]thiadiazine 1,1- dioxide |
| 6-8 | 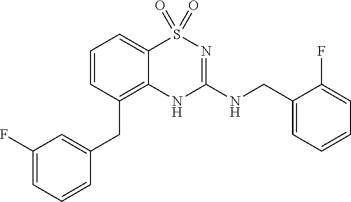 [SMILES: O=S1(=O)N=C(NCc2ccccc2F)Nc2c(Cc3cccc(F)c3)cccc21] | 5-(3-fluorobenzyl)-3-((2- fluorobenzyl)amino)-4H- benzo[e][1,2,4]thiadiazine 1,1- dioxide |
| 6-9 | 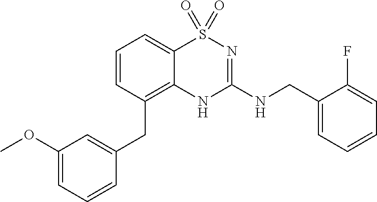 [SMILES: COc1cccc(Cc2cccc3c2NC(NCc2ccccc2F)=NS3(=O)=O)c1] | 3-((2-fluorobenzyl)amino)-5-(3- methoxybenzyl)-4H- benzo[e][1,2,4]thiadiazine 1,1- dioxide |
| 6-10 | 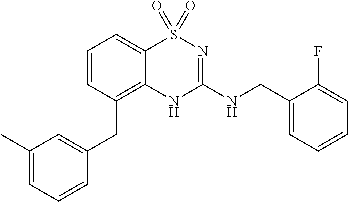 [SMILES: Cc1cccc(Cc2cccc3c2NC(NCc2ccccc2F)=NS3(=O)=O)c1] | 3-((2-fluorobenzyl)amino)-5-(3- methylbenzyl)-4H- benzo[e][1,2,4]thiadiazine 1,1- dioxide |
| 6-11 | 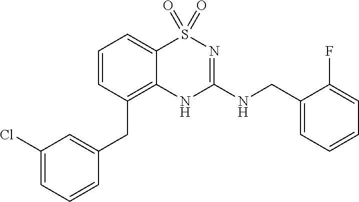 [SMILES: O=S1(=O)N=C(NCc2ccccc2F)Nc2c(Cc3cccc(Cl)c3)cccc21] | 5-(3-chlorobenzyl)-3-((2- fluorobenzyl)amino)-4H- benzo[e][1,2,4]thiadiazine 1,1- dioxide |
| 6-12 | 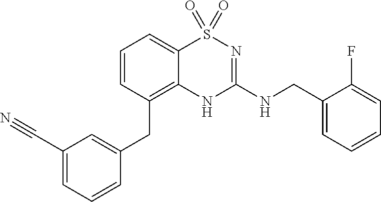 [SMILES: N#Cc1cccc(Cc2cccc3c2NC(NCc2ccccc2F)=NS3(=O)=O)c1] | 3-((3-((2-fluorobenzyl)amino)-1,1- dioxido-4H- benzo[e][1,2,4]thiadiazin-5- yl)methyl)benzonitrile |
| 6-13 | 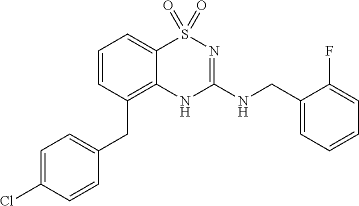 [SMILES: O=S1(=O)N=C(NCc2ccccc2F)Nc2c(Cc3ccc(Cl)cc3)cccc21] | 5-(4-chlorobenzyl)-3-((2- fluorobenzyl)amino)-4H- benzo[e][1,2,4]thiadiazine 1,1- dioxide |
| 6-14 | 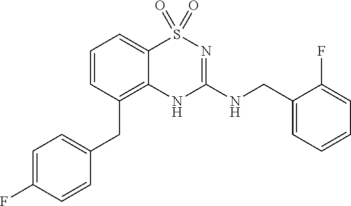 [SMILES: O=S1(=O)N=C(NCc2ccccc2F)Nc2c(Cc3ccc(F)cc3)cccc21] | 5-(4-fluorobenzyl)-3-((2- fluorobenzyl)amino)-4H- benzo[e][1,2,4]thiadiazine 1,1- dioxide |
| 6-15 | 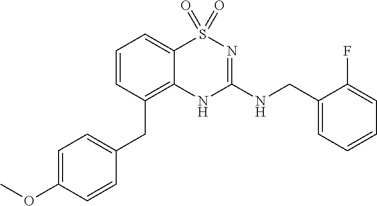 [SMILES: COc1ccc(Cc2cccc3c2NC(NCc2ccccc2F)=NS3(=O)=O)cc1] | 3-((2-fluorobenzyl)amino)-5-(4- methoxybenzyl)-4H- benzo[e][1,2,4]thiadiazine 1,1- dioxide |
| 6-16 | 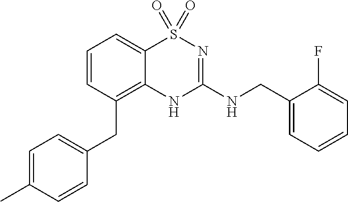 [SMILES: Cc1ccc(Cc2cccc3c2NC(NCc2ccccc2F)=NS3(=O)=O)cc1] | 3-((2-fluorobenzyl)amino)-5-(4- methylbenzyl)-4H- benzo[e][1,2,4]thiadiazine 1,1- dioxide |
| 6-17 | 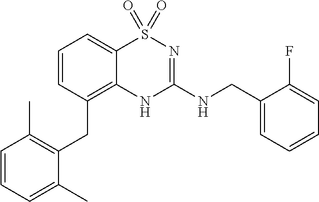 [SMILES: Cc1cccc(C)c1Cc1cccc2c1NC(NCc1ccccc1F)=NS2(=O)=O] | 5-(2,6-dimethylbenzyl)-3-((2- fluorobenzyl)amino)-4H- benzo[e][1,2,4]thiadiazine 1,1- dioxide |
| 6-18 | 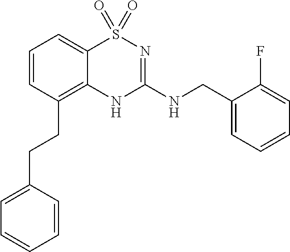 [SMILES: O=S1(=O)N=C(NCc2ccccc2F)Nc2c(CCc3ccccc3)cccc21] | 3-((2-fluorobenzyl)amino)-5- phenethyl-4H- benzo[e][1,2,4]thiadiazine 1,1- dioxide |
| 6-19 | 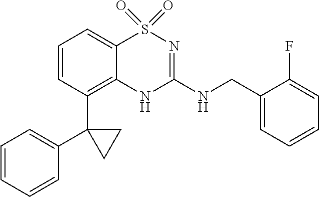 [SMILES: O=S1(=O)N=C(NCc2ccccc2F)Nc2c(C3(c4ccccc4)CC3)cccc21] | 3-((2-fluorobenzyl)amino)-5-(1- phenylcyclopropyl)-4H- benzo[e][1,2,4]thiadiazine 1,1- dioxide |
| 7-1 | 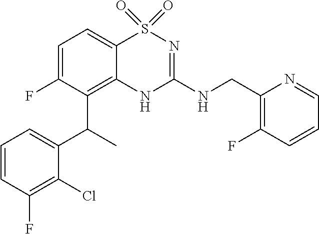 [SMILES: CC(c1cccc(F)c1Cl)c1c(F)ccc2c1NC(NCc1ncccc1F)=NS2(=O)=O] | 5-(1-(2-chloro-3- fluorophenyl)ethyl)-6-fluoro-3- (((3-fluoropyridin-2- yl)methyl)amino)-4H- benzo[e][1,2,4]thiadiazine 1,1- dioxide |
| 7-1 (S) | 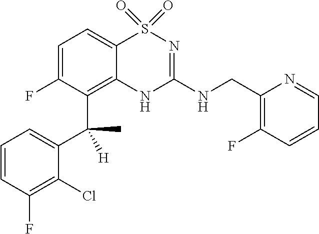 [SMILES: C[C@@H](c1cccc(F)c1Cl)c1c(F)ccc2c1NC(NCc1ncccc1F)=NS2(=O)=O] | (S)-5-(1-(2-chloro-3- fluorophenyl)ethyl)-6-fluoro-3- (((3-fluoropyridin-2- yl)methyl)amino)-4H- benzo[e][1,2,4]thiadiazine 1,1- dioxide |
| 7-1 (R) | 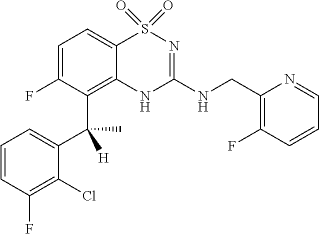 [SMILES: C[C@H](c1cccc(F)c1Cl)c1c(F)ccc2c1NC(NCc1ncccc1F)=NS2(=O)=O] | (R)-5-(1-(2-chloro-3- fluorophenyl)ethyl)-6-fluoro-3- (((3-fluoropyridin-2- yl)methyl)amino)-4H- benzo[e][1,2,4]thiadiazine 1,1- dioxide |
| 7-2 | 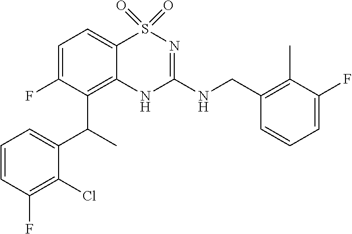 [SMILES: Cc1c(F)cccc1CNC1=NS(=O)(=O)c2ccc(F)c(C(C)c3cccc(F)c3Cl)c2N1] | 5-(1-(2-chloro-3- fluorophenyl)ethyl)-6-fluoro-3-((3- fluoro-2-methylbenzyl)amino)-4H- benzo[e][1,2,4]thiadiazine 1,1- dioxide |
| 7-2 (S) | 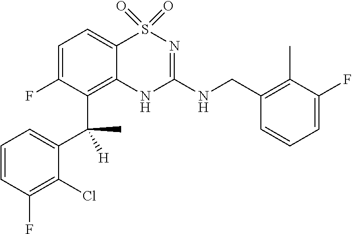 [SMILES: Cc1c(F)cccc1CNC1=NS(=O)(=O)c2ccc(F)c([C@@H](C)c3cccc(F)c3Cl)c2N1] | (S)-5-(1-(2-chloro-3- fluorophenyl)ethyl)-6-fluoro-3-((3- fluoro-2-methylbenzyl)amino)-4H- benzo[e][1,2,4]thiadiazine 1,1- dioxide |
| 7-2 (R) | 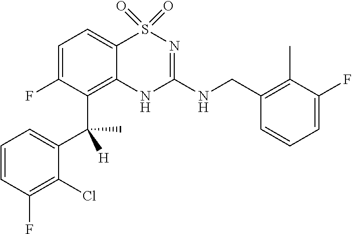 [SMILES: Cc1c(F)cccc1CNC1=NS(=O)(=O)c2ccc(F)c([C@H](C)c3cccc(F)c3Cl)c2N1] | (R)-5-(1-(2-chloro-3- fluorophenyl)ethyl)-6-fluoro-3-((3- fluoro-2-methylbenzyl)amino)-4H- benzo[e][1,2,4]thiadiazine 1,1- dioxide |
| 7-3 | 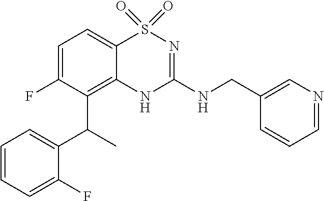 [SMILES: CC(c1ccccc1F)c1c(F)ccc2c1NC(NCc1cccnc1)=NS2(=O)=O] | 6-fluoro-5-(1-(2- fluorophenyl)ethyl)-3-((pyridin-3- ylmethyl)amino)-4H- benzo[e][1,2,4]thiadiazine 1,1- dioxide |
| 7-3 (S) | 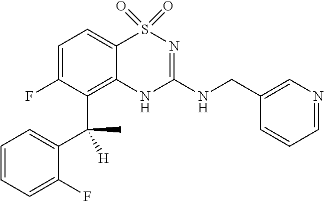 [SMILES: C[C@@H](c1ccccc1F)c1c(F)ccc2c1NC(NCc1cccnc1)=NS2(=O)=O] | (S)-6-fluoro-5-(1-(2- fluorophenyl)ethyl)-3-((pyridin-3- ylmethyl)amino)-4H- benzo[e][1,2,4]thiadiazine 1,1- dioxide |
| 7-3 (R) | 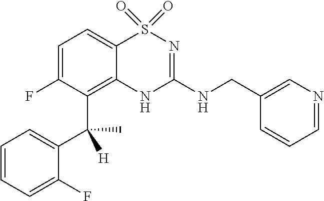 [SMILES: C[C@H](c1ccccc1F)c1c(F)ccc2c1NC(NCc1cccnc1)=NS2(=O)=O] | (R)-6-fluoro-5-(1-(2- fluorophenyl)ethyl)-3-((pyridin-3- ylmethyl)amino)-4H- benzo[e][1,2,4]thiadiazine 1,1- dioxide |
| 7-4 | 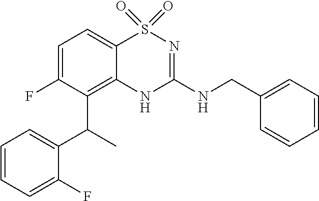 [SMILES: CC(c1ccccc1F)c1c(F)ccc2c1NC(NCc1ccccc1)=NS2(=O)=O] | 3-(benzylamino)-6-fluoro-5-(1-(2- fluorophenyl)ethyl)-4H- benzo[e][1,2,4]thiadiazine 1,1- dioxide |
| 7-4 (S) | 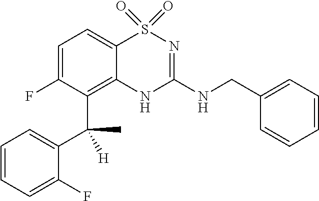 [SMILES: C[C@@H](c1ccccc1F)c1c(F)ccc2c1NC(NCc1ccccc1)=NS2(=O)=O] | (S)-3-(benzylamino)-6-fluoro-5-(1- (2-fluorophenyl)ethyl)-4H- benzo[e][1,2,4]thiadiazine 1,1- dioxide |
| 7-4 (R) | 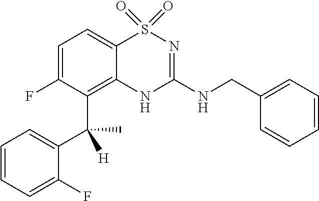 [SMILES: C[C@H](c1ccccc1F)c1c(F)ccc2c1NC(NCc1ccccc1)=NS2(=O)=O] | (R)-3-(benzylamino)-6-fluoro-5-(1- (2-fluorophenyl)ethyl)-4H- benzo[e][1,2,4]thiadiazine 1,1- dioxide |
| 7-5 | 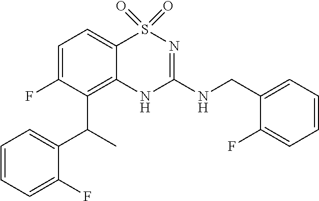 [SMILES: CC(c1ccccc1F)c1c(F)ccc2c1NC(NCc1ccccc1F)=NS2(=O)=O] | 6-fluoro-3-((2- fluorobenzyl)amino)-5-(1-(2- fluorophenyl)ethyl)-4H- benzo[e][1,2,4]thiadiazine 1,1- dioxide |
| 7-5 (S) | 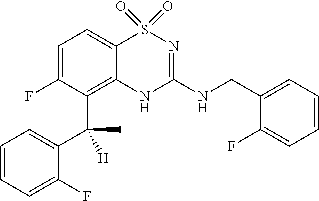 [SMILES: C[C@@H](c1ccccc1F)c1c(F)ccc2c1NC(NCc1ccccc1F)=NS2(=O)=O] | (S)-6-fluoro-3-((2- fluorobenzyl)amino)-5-(1-(2- fluorophenyl)ethyl)-4H- benzo[e][1,2,4]thiadiazine 1,1- dioxide |
| 7-5 (R) | 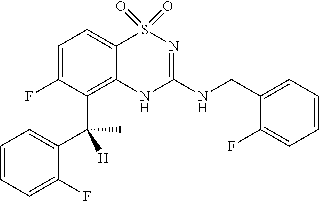 [SMILES: C[C@H](c1ccccc1F)c1c(F)ccc2c1NC(NCc1ccccc1F)=NS2(=O)=O] | (R)-6-fluoro-3-((2- fluorobenzyl)amino)-5-(1-(2- fluorophenyl)ethyl)-4H- benzo[e][1,2,4]thiadiazine 1,1- dioxide |
| 7-6 | 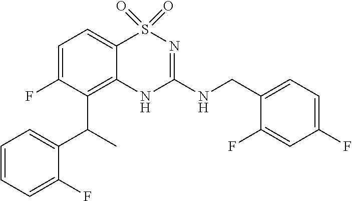 [SMILES: CC(c1ccccc1F)c1c(F)ccc2c1NC(NCc1ccc(F)cc1F)=NS2(=O)=O] | 3-((2,4-difluorobenzyl)amino)-6- fluoro-5-(1-(2-fluorophenyl)ethyl)- 4H-benzo[e][1,2,4]thiadiazine 1,1- dioxide |
| 7-6 (S) | 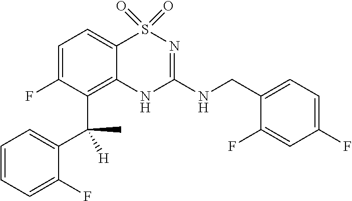 [SMILES: C[C@@H](c1ccccc1F)c1c(F)ccc2c1NC(NCc1ccc(F)cc1F)=NS2(=O)=O] | (S)-3-((2,4-difluorobenzyl)amino)- 6-fluoro-5-(1-(2- fluorophenyl)ethyl)-4H- benzo[e][1,2,4]thiadiazine 1,1- dioxide |
| 7-6 (R) | 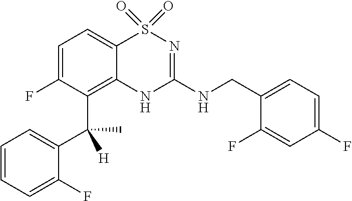 [SMILES: C[C@H](c1ccccc1F)c1c(F)ccc2c1NC(NCc1ccc(F)cc1F)=NS2(=O)=O] | (R)-3-((2,4-difluorobenzyl)amino)- 6-fluoro-5-(1-(2- fluorophenyl)ethyl)-4H- benzo[e][1,2,4]thiadiazine 1,1- dioxide |
| 7-7 | 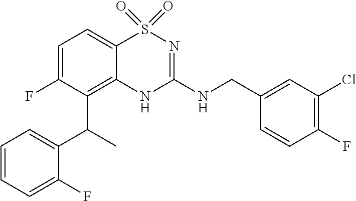 [SMILES: CC(c1ccccc1F)c1c(F)ccc2c1NC(NCc1ccc(F)c(Cl)c1)=NS2(=O)=O] | 3-((3-chloro-4- fluorobenzyl)amino)-6-fluoro-5-(1- (2-fluorophenyl)ethyl)-4H- benzo[e][1,2,4]thiadiazine 1,1- dioxide |
| 7-7 (S) | 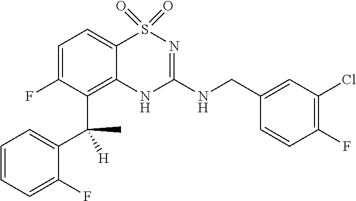 [SMILES: C[C@@H](c1ccccc1F)c1c(F)ccc2c1NC(NCc1ccc(F)c(Cl)c1)=NS2(=O)=O] | (S)- 3-((3-chloro-4- fluorobenzyl)amino)-6-fluoro-5-(1- (2-fluorophenyl)ethyl)-4H- benzo[e][1,2,4]thiadiazine 1,1- dioxide |
| 7-7 (R) | 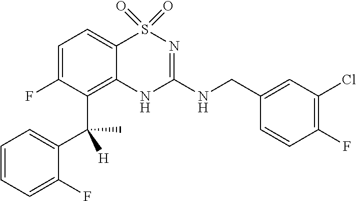 [SMILES: C[C@H](c1ccccc1F)c1c(F)ccc2c1NC(NCc1ccc(F)c(Cl)c1)=NS2(=O)=O] | (R)-3-((3-chloro-4- fluorobenzyl)amino)-6-fluoro-5-(1- (2-fluorophenyl)ethyl)-4H- benzo[e][1,2,4]thiadiazine 1,1- dioxide |
| 7-8 | 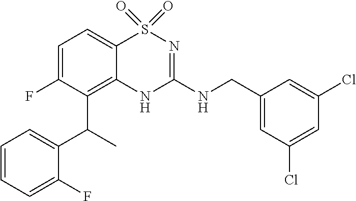 [SMILES: CC(c1ccccc1F)c1c(F)ccc2c1NC(NCc1cc(Cl)cc(Cl)c1)=NS2(=O)=O] | 3-((3,5-dichlorobenzyl)amino)-6- fluoro-5-(1-(2-fluorophenyl)ethyl)- 4H-benzo[e][1,2,4]thiadiazine 1,1- dioxide |
| 7-8 (S) | 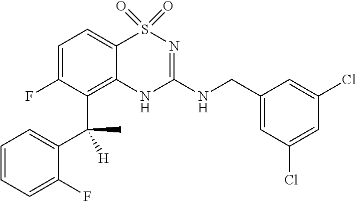 [SMILES: C[C@@H](c1ccccc1F)c1c(F)ccc2c1NC(NCc1cc(Cl)cc(Cl)c1)=NS2(=O)=O] | (S)-3-((3,5-dichlorobenzyl)amino)- 6-fluoro-5-(1-(2- fluorophenyl)ethyl)-4H- benzo[e][1,2,4]thiadiazine 1,1- dioxide |
| 7-8 (R) | 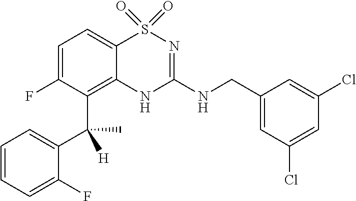 [SMILES: C[C@H](c1ccccc1F)c1c(F)ccc2c1NC(NCc1cc(Cl)cc(Cl)c1)=NS2(=O)=O] | (R)-3-((3,5-dichlorobenzyl)amino)- 6-fluoro-5-(1-(2- fluorophenyl)ethyl)-4H- benzo[e][1,2,4]thiadiazine 1,1- dioxide |
| 7-9 | 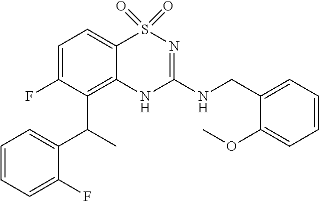 [SMILES: COc1ccccc1CNC1=NS(=O)(=O)c2ccc(F)c(C(C)c3ccccc3F)c2N1] | 6-fluoro-5-(1-(2- fluorophenyl)ethyl)-3-((2- methoxybenzyl)amino)-4H- benzo[e][1,2,4]thiadiazine 1,1- dioxide |
| 7-9 (S) | 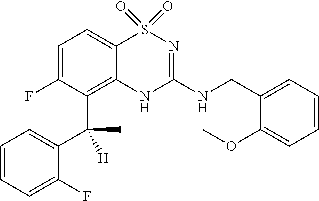 [SMILES: COc1ccccc1CNC1=NS(=O)(=O)c2ccc(F)c([C@@H](C)c3ccccc3F)c2N1] | (S)-6-fluoro-5-(1-(2- fluorophenyl)ethyl)-3-((2- methoxybenzyl)amino)-4H- benzo[e][1,2,4]thiadiazine 1,1- dioxide |
| 7-9 (R) | 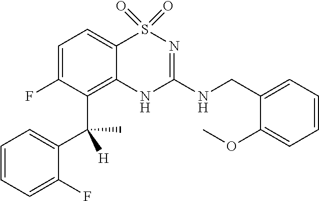 [SMILES: COc1ccccc1CNC1=NS(=O)(=O)c2ccc(F)c([C@H](C)c3ccccc3F)c2N1] | (R)-6-fluoro-5-(1-(2- fluorophenyl)ethyl)-3-((2- methoxybenzyl)amino)-4H- benzo[e][1,2,4]thiadiazine 1,1- dioxide |
| 7-10 | 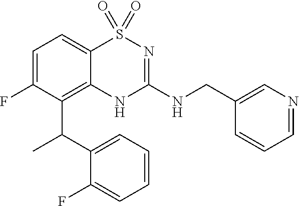 [SMILES: CC(c1ccccc1F)c1c(F)ccc2c1NC(NCc1cccnc1)=NS2(=O)=O] | 6-fluoro-5-(1-(2- fluorophenyl)ethyl)-3-((pyridin-3- ylmethyl)amino)-4H- benzo[e][1,2,4]thiadiazine 1,1- dioxide |
| 7-10 (S) | 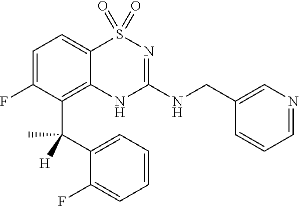 [SMILES: C[C@@H](c1ccccc1F)c1c(F)ccc2c1NC(NCc1cccnc1)=NS2(=O)=O] | (S)- 6-fluoro-5-(1-(2- fluorophenyl)ethyl)-3-((pyridin-3- ylmethyl)amino)-4H- benzo[e][1,2,4]thiadiazine 1,1- dioxide |
| 7-10 (R) | 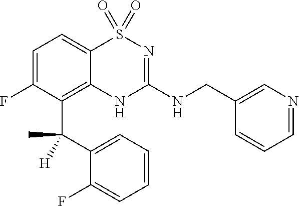 [SMILES: C[C@H](c1ccccc1F)c1c(F)ccc2c1NC(NCc1cccnc1)=NS2(=O)=O] | (R)-6-fluoro-5-(1-(2- fluorophenyl)ethyl)-3-((pyridin-3- ylmethyl)amino)-4H- benzo[e][1,2,4]thiadiazine 1,1- dioxide |
| 7-11 | 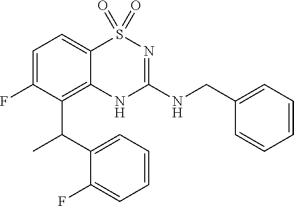 [SMILES: CC(c1ccccc1F)c1c(F)ccc2c1NC(NCc1ccccc1)=NS2(=O)=O] | 3-(benzylamino)-6-fluoro-5-(1-(2- fluorophenyl)ethyl)-4H- benzo[e][1,2,4]thiadiazine 1,1- dioxide |
| 7-11 (S) | 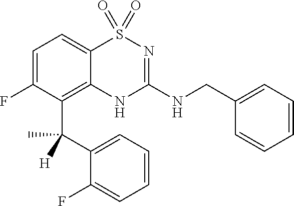 [SMILES: C[C@@H](c1ccccc1F)c1c(F)ccc2c1NC(NCc1ccccc1)=NS2(=O)=O] | (S)-3-(benzylamino)-6-fluoro-5-(1- (2-fluorophenyl)ethyl)-4H- benzo[e][1,2,4]thiadiazine 1,1- dioxide |
| 7-11 (R) | 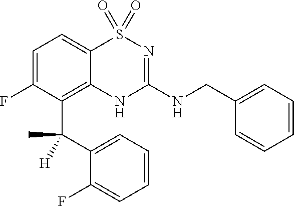 [SMILES: C[C@H](c1ccccc1F)c1c(F)ccc2c1NC(NCc1ccccc1)=NS2(=O)=O] | (R)-3-(benzylamino)-6-fluoro-5-(1- (2-fluorophenyl)ethyl)-4H- benzo[e][1,2,4]thiadiazine 1,1- dioxide |
| 7-12 | 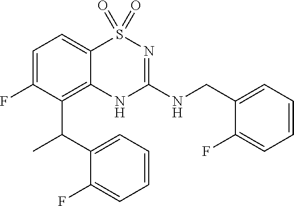 [SMILES: CC(c1ccccc1F)c1c(F)ccc2c1NC(NCc1ccccc1F)=NS2(=O)=O] | 6-fluoro-3-((2- fluorobenzyl)amino)-5-(1-(2- fluorophenyl)ethyl)-4H- benzo[e][1,2,4]thiadiazine 1,1- dioxide |
| 7-12 (S) | 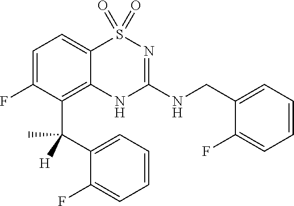 [SMILES: C[C@@H](c1ccccc1F)c1c(F)ccc2c1NC(NCc1ccccc1F)=NS2(=O)=O] | (S)-6-fluoro-3-((2- fluorobenzyl)amino)-5-(1-(2- fluorophenyl)ethyl)-4H- benzo[e][1,2,4]thiadiazine 1,1- dioxide |
| 7-12 (R) | 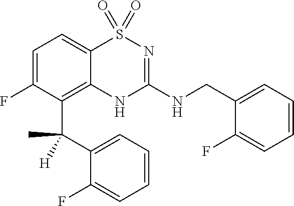 [SMILES: C[C@H](c1ccccc1F)c1c(F)ccc2c1NC(NCc1ccccc1F)=NS2(=O)=O] | (R)-6-fluoro-3-((2- fluorobenzyl)amino)-5-(1-(2- fluorophenyl)ethyl)-4H- benzo[e][1,2,4]thiadiazine 1,1- dioxide |
| 7-13 | 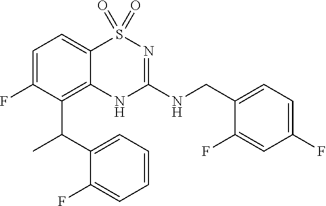 [SMILES: CC(c1ccccc1F)c1c(F)ccc2c1NC(NCc1ccc(F)cc1F)=NS2(=O)=O] | 3-((2,4-difluorobenzyl)amino)-6- fluoro-5-(1-(2-fluorophenyl)ethyl)- 4H-benzo[e][1,2,4]thiadiazine 1,1- dioxide |
| 7-13 (S) | 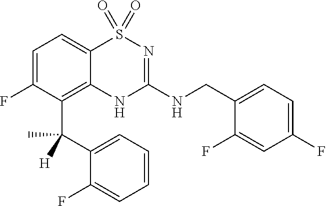 [SMILES: C[C@@H](c1ccccc1F)c1c(F)ccc2c1NC(NCc1ccc(F)cc1F)=NS2(=O)=O] | (S)-3-((2,4-difluorobenzyl)amino)- 6-fluoro-5-(1-(2- fluorophenyl)ethyl)-4H- benzo[e][1,2,4]thiadiazine 1,1- dioxide |
| 7-13 (R) | 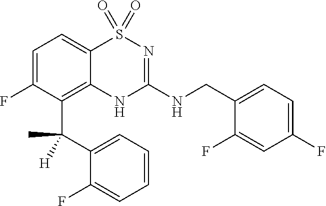 [SMILES: C[C@H](c1ccccc1F)c1c(F)ccc2c1NC(NCc1ccc(F)cc1F)=NS2(=O)=O] | (R)-3-((2,4-difluorobenzyl)amino)- 6-fluoro-5-(1-(2- fluorophenyl)ethyl)-4H- benzo[e][1,2,4]thiadiazine 1,1- dioxide |
| 7-14 | 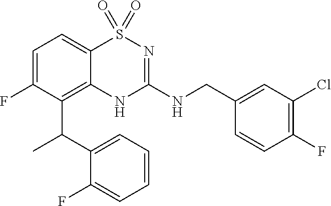 [SMILES: CC(c1ccccc1F)c1c(F)ccc2c1NC(NCc1ccc(F)c(Cl)c1)=NS2(=O)=O] | 3-((3-chloro-4- fluorobenzyl)amino)-6-fluoro-5-(1- (2-fluorophenyl)ethyl)-4H- benzo[e][1,2,4]thiadiazine 1,1- dioxide |
| 7-14 (S) | 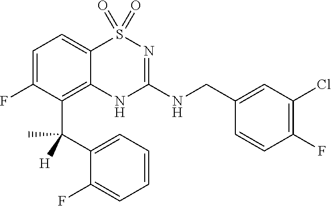 [SMILES: C[C@@H](c1ccccc1F)c1c(F)ccc2c1NC(NCc1ccc(F)c(Cl)c1)=NS2(=O)=O] | (S)- 3-((3-chloro-4- fluorobenzyl)amino)-6-fluoro-5-(1- (2-fluorophenyl)ethyl)-4H- benzo[e][1,2,4]thiadiazine 1,1- dioxide |
| 7-14 (R) | 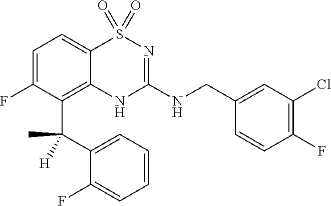 [SMILES: C[C@H](c1ccccc1F)c1c(F)ccc2c1NC(NCc1ccc(F)c(Cl)c1)=NS2(=O)=O] | (R)-3-((3-chloro-4- fluorobenzyl)amino)-6-fluoro-5-(1- (2-fluorophenyl)ethyl)-4H- benzo[e][1,2,4]thiadiazine 1,1- dioxide |
| 7-15 | 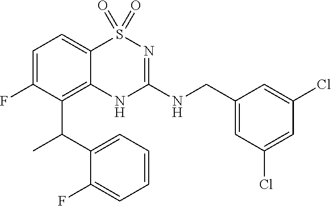 [SMILES: CC(c1ccccc1F)c1c(F)ccc2c1NC(NCc1cc(Cl)cc(Cl)c1)=NS2(=O)=O] | 3-((3,5-dichlorobenzyl)amino)-6- fluoro-5-(1-(2-fluorophenyl)ethyl)- 4H-benzo[e][1,2,4]thiadiazine 1,1- dioxide |
| 7-15 (S) | 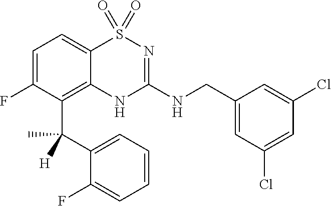 [SMILES: C[C@@H](c1ccccc1F)c1c(F)ccc2c1NC(NCc1cc(Cl)cc(Cl)c1)=NS2(=O)=O] | (S)-3-((3,5-dichlorobenzyl)amino)- 6-fluoro-5-(1-(2- fluorophenyl)ethyl)-4H- benzo[e][1,2,4]thiadiazine 1,1- dioxide |
| 7-15 (R) | 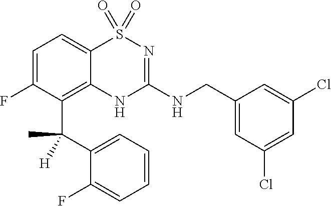 [SMILES: C[C@H](c1ccccc1F)c1c(F)ccc2c1NC(NCc1cc(Cl)cc(Cl)c1)=NS2(=O)=O] | (R)-3-((3,5-dichlorobenzyl)amino)- 6-fluoro-5-(1-(2- fluorophenyl)ethyl)-4H- benzo[e][1,2,4]thiadiazine 1,1- dioxide |
| 7-16 | 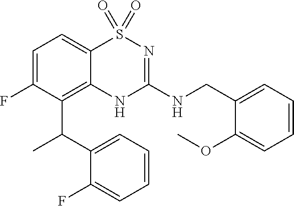 [SMILES: COc1ccccc1CNC1=NS(=O)(=O)c2ccc(F)c(C(C)c3ccccc3F)c2N1] | 6-fluoro-5-(1-(2- fluorophenyl)ethyl)-3-((2- methoxybenzyl)amino)-4H- benzo[e][1,2,4]thiadiazine 1,1- dioxide |
| 7-16 (S) | 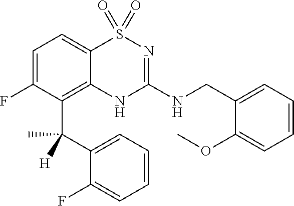 [SMILES: COc1ccccc1CNC1=NS(=O)(=O)c2ccc(F)c([C@@H](C)c3ccccc3F)c2N1] | (S)-6-fluoro-5-(1-(2- fluorophenyl)ethyl)-3-((2- methoxybenzyl)amino)-4H- benzo[e][1,2,4]thiadiazine 1,1- dioxide |
| 7-16 (R) | 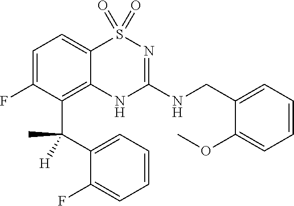 [SMILES: COc1ccccc1CNC1=NS(=O)(=O)c2ccc(F)c([C@H](C)c3ccccc3F)c2N1] | (R)-6-fluoro-5-(1-(2- fluorophenyl)ethyl)-3-((2- methoxybenzyl)amino)-4H- benzo[e][1,2,4]thiadiazine 1,1- dioxide |
| 7-17 | 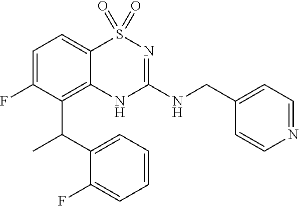 [SMILES: CC(c1ccccc1F)c1c(F)ccc2c1NC(NCc1ccncc1)=NS2(=O)=O] | 6-fluoro-5-(1-(2- fluorophenyl)ethyl)-3-((pyridin-4- ylmethyl)amino)-4H- benzo[e][1,2,4]thiadiazine 1,1- dioxide |
| 7-17 (S) | 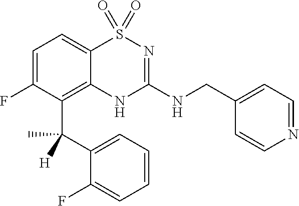 [SMILES: C[C@@H](c1ccccc1F)c1c(F)ccc2c1NC(NCc1ccncc1)=NS2(=O)=O] | (S)-6-fluoro-5-(1-(2- fluorophenyl)ethyl)-3-((pyridin-4- ylmethyl)amino)-4H- benzo[e][1,2,4]thiadiazine 1,1- dioxide |
| 7-17 (R) | 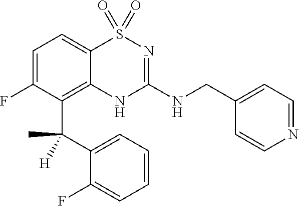 [SMILES: C[C@H](c1ccccc1F)c1c(F)ccc2c1NC(NCc1ccncc1)=NS2(=O)=O] | (R)-6-fluoro-5-(1-(2- fluorophenyl)ethyl)-3-((pyridin-4- ylmethyl)amino)-4H- benzo[e][1,2,4]thiadiazine 1,1- dioxide |
| 7-18 | 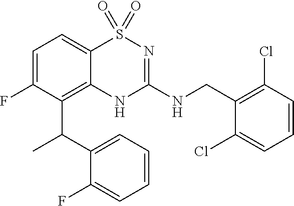 [SMILES: CC(c1ccccc1F)c1c(F)ccc2c1NC(NCc1c(Cl)cccc1Cl)=NS2(=O)=O] | 3-((2,6-dichlorobenzyl)amino)-6- fluoro-5-(1-(2-fluorophenyl)ethyl)- 4H-benzo[e][1,2,4]thiadiazine 1,1- dioxide |
| 7-18 (S) | 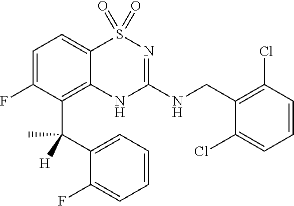 [SMILES: C[C@@H](c1ccccc1F)c1c(F)ccc2c1NC(NCc1c(Cl)cccc1Cl)=NS2(=O)=O] | (S)-3-((2,6-dichlorobenzyl)amino)- 6-fluoro-5-(1-(2- fluorophenyl)ethyl)-4H- benzo[e][1,2,4]thiadiazine 1,1- dioxide |
| 7-18 (R) | 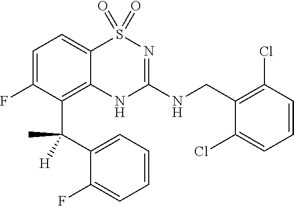 [SMILES: C[C@H](c1ccccc1F)c1c(F)ccc2c1NC(NCc1c(Cl)cccc1Cl)=NS2(=O)=O] | (R)-3-((2,6-dichlorobenzyl)amino)- 6-fluoro-5-(1-(2- fluorophenyl)ethyl)-4H- benzo[e][1,2,4]thiadiazine 1,1- dioxide |
| 7-19 | 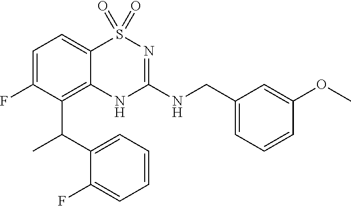 [SMILES: COc1cccc(CNC2=NS(=O)(=O)c3ccc(F)c(C(C)c4ccccc4F)c3N2)c1] | 6-fluoro-5-(1-(2- fluorophenyl)ethyl)-3-((3- methoxybenzyl)amino)-4H- benzo[e][1,2,4]thiadiazine 1,1- dioxide |
| 7-19 (S) | 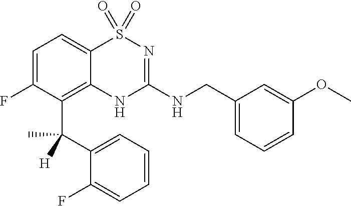 [SMILES: COc1cccc(CNC2=NS(=O)(=O)c3ccc(F)c([C@@H](C)c4ccccc4F)c3N2)c1] | (S)-6-fluoro-5-(1-(2- fluorophenyl)ethyl)-3-((3- methoxybenzyl)amino)-4H- benzo[e][1,2,4]thiadiazine 1,1- dioxide |
| 7-19 (R) | 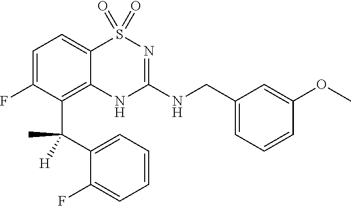 [SMILES: COc1cccc(CNC2=NS(=O)(=O)c3ccc(F)c([C@H](C)c4ccccc4F)c3N2)c1] | (R)-6-fluoro-5-(1-(2- fluorophenyl)ethyl)-3-((3- methoxybenzyl)amino)-4H- benzo[e][1,2,4]thiadiazine 1,1- dioxide |
| 7-20 | 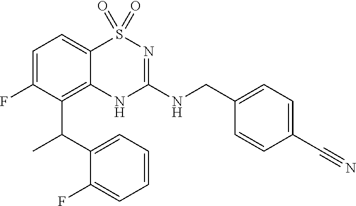 [SMILES: CC(c1ccccc1F)c1c(F)ccc2c1NC(NCc1ccc(C#N)cc1)=NS2(=O)=O] | 4-(((6-fluoro-5-(1-(2- fluorophenyl)ethyl)-1,1-dioxido- 4H-benzo[e][1,2,4]thiadiazin-3- yl)amino)methyl)benzonitrile |
| 7-20 (S) | 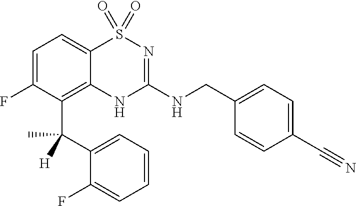 [SMILES: C[C@@H](c1ccccc1F)c1c(F)ccc2c1NC(NCc1ccc(C#N)cc1)=NS2(=O)=O] | (S)-4-(((6-fluoro-5-(1-(2- fluorophenyl)ethyl)-1,1-dioxido- 4H-benzo[e][1,2,4]thiadiazin-3- yl)amino)methyl)benzonitrile |
| 7-20 (R) | 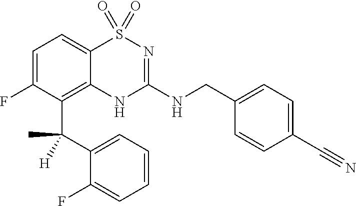 [SMILES: C[C@H](c1ccccc1F)c1c(F)ccc2c1NC(NCc1ccc(C#N)cc1)=NS2(=O)=O] | (R)-4-(((6-fluoro-5-(1-(2- fluorophenyl)ethyl)-1,1-dioxido- 4H-benzo[e][1,2,4]thiadiazin-3- yl)amino)methyl)benzonitrile |
| 7-21 | 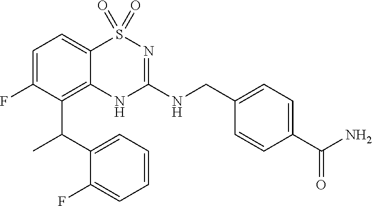 [SMILES: CC(c1ccccc1F)c1c(F)ccc2c1NC(NCc1ccc(C(N)=O)cc1)=NS2(=O)=O] | 4-(((6-fluoro-5-(1-(2- fluorophenyl)ethyl)-1,1-dioxido- 4H-benzo[e][1,2,4]thiadiazin-3- yl)amino)methyl)benzamide |
| 7-21 (S) | 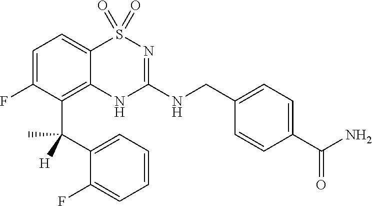 [SMILES: C[C@@H](c1ccccc1F)c1c(F)ccc2c1NC(NCc1ccc(C(N)=O)cc1)=NS2(=O)=O] | (S)-4-(((6-fluoro-5-(1-(2- fluorophenyl)ethyl)-1,1-dioxido- 4H-benzo[e][1,2,4]thiadiazin-3- yl)amino)methyl)benzamide |
| 7-21 (R) | 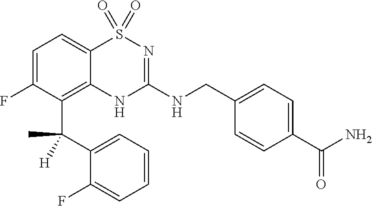 [SMILES: C[C@H](c1ccccc1F)c1c(F)ccc2c1NC(NCc1ccc(C(N)=O)cc1)=NS2(=O)=O] | (R)-4-(((6-fluoro-5-(1-(2- fluorophenyl)ethyl)-1,1-dioxido- 4H-benzo[e][1,2,4]thiadiazin-3- yl)amino)methyl)benzamide |
| 7-22 | 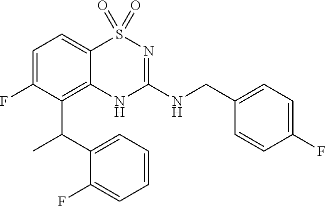 [SMILES: CC(c1ccccc1F)c1c(F)ccc2c1NC(NCc1ccc(F)cc1)=NS2(=O)=O] | 6-fluoro-3-((4- fluorobenzyl)amino)-5-(1-(2- fluorophenyl)ethyl)-4H- benzo[e][1,2,4]thiadiazine 1,1- dioxide |
| 7-22 (S) | 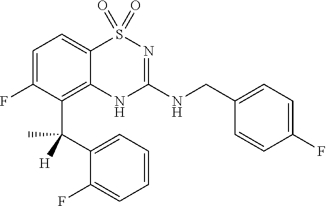 [SMILES: C[C@@H](c1ccccc1F)c1c(F)ccc2c1NC(NCc1ccc(F)cc1)=NS2(=O)=O] | (S)-6-fluoro-3-((4- fluorobenzyl)amino)-5-(1-(2- fluorophenyl)ethyl)-4H- benzo[e][1,2,4]thiadiazine 1,1- dioxide |
| 7-22 (R) | 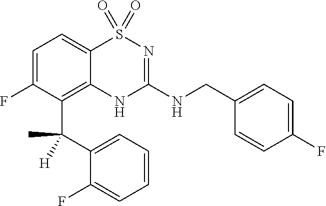 [SMILES: C[C@H](c1ccccc1F)c1c(F)ccc2c1NC(NCc1ccc(F)cc1)=NS2(=O)=O] | (R)-6-fluoro-3-((4- fluorobenzyl)amino)-5-(1-(2- fluorophenyl)ethyl)-4H- benzo[e][1,2,4]thiadiazine 1,1- dioxide |
| 7-23 | 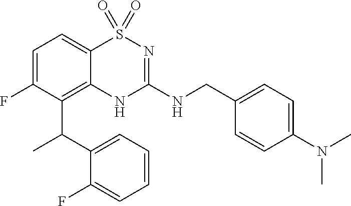 [SMILES: CC(c1ccccc1F)c1c(F)ccc2c1NC(NCc1ccc(N(C)C)cc1)=NS2(=O)=O] | 3-((4- (dimethylamino)benzyl)amino)-6- fluoro-5-(1-(2-fluorophenyl)ethyl)- 4H-benzo[e][1,2,4]thiadiazine 1,1- dioxide |
| 7-23 (S) | 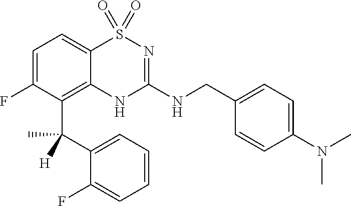 [SMILES: C[C@@H](c1ccccc1F)c1c(F)ccc2c1NC(NCc1ccc(N(C)C)cc1)=NS2(=O)=O] | (S)-3-((4- (dimethylamino)benzyl)amino)-6- fluoro-5-(1-(2-fluorophenyl)ethyl)- 4H-benzo[e][1,2,4]thiadiazine 1,1- dioxide |
| 7-23 (R) | 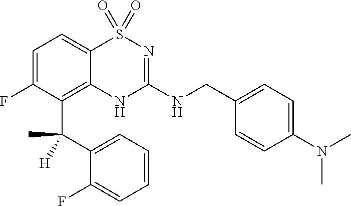 [SMILES: C[C@H](c1ccccc1F)c1c(F)ccc2c1NC(NCc1ccc(N(C)C)cc1)=NS2(=O)=O] | (R)-3-((4- (dimethylamino)benzyl)amino)-6- fluoro-5-(1-(2-fluorophenyl)ethyl)- 4H-benzo[e][1,2,4]thiadiazine 1,1- dioxide |
| 7-24 | 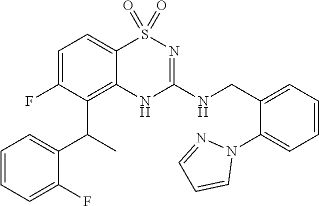 [SMILES: CC(c1ccccc1F)c1c(F)ccc2c1NC(NCc1ccccc1-n1cccn1)=NS2(=O)=O] | 3-((2-(1H-pyrazol-1- yl)benzyl)amino)-6-fluoro-5-(1-(2- fluorophenyl)ethyl)-4H- benzo[e][1,2,4]thiadiazine 1,1- dioxide |
| 7-24 (S) | 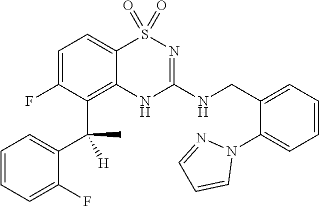 [SMILES: C[C@@H](c1ccccc1F)c1c(F)ccc2c1NC(NCc1ccccc1-n1cccn1)=NS2(=O)=O] | (S)-3-((2-(1H-pyrazol-1- yl)benzyl)amino)-6-fluoro-5-(1-(2- fluorophenyl)ethyl)-4H- benzo[e][1,2,4]thiadiazine 1,1- dioxide |
| 7-24 (R) | 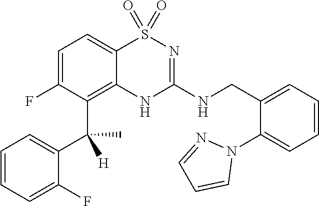 [SMILES: C[C@H](c1ccccc1F)c1c(F)ccc2c1NC(NCc1ccccc1-n1cccn1)=NS2(=O)=O] | (R)-3-((2-(1H-pyrazol-1- yl)benzyl)amino)-6-fluoro-5-(1-(2- fluorophenyl)ethyl)-4H- benzo[e][1,2,4]thiadiazine 1,1- dioxide |
| 7-25 | 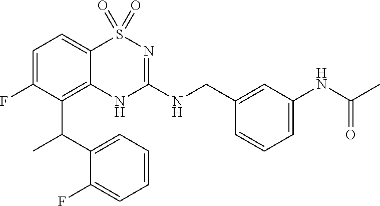 [SMILES: CC(=O)Nc1cccc(CNC2=NS(=O)(=O)c3ccc(F)c(C(C)c4ccccc4F)c3N2)c1] | N-(3-(((6-fluoro-5-(1-(2- fluorophenyl)ethyl)-1,1-dioxido- 4H-benzo[e][1,2,4]thiadiazin-3- yl)amino)methyl)phenyl)acetamide |
| 7-25 (S) | 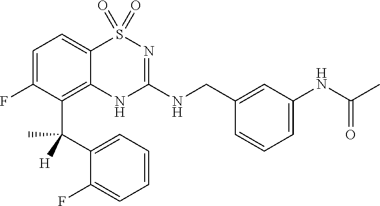 [SMILES: CC(=O)Nc1cccc(CNC2=NS(=O)(=O)c3ccc(F)c([C@@H](C)c4ccccc4F)c3N2)c1] | (S)-N-(3-(((6-fluoro-5-(1-(2- fluorophenyl)ethyl)-1,1-dioxido- 4H-benzo[e][1,2,4]thiadiazin-3- yl)amino)methyl)phenyl)acetamide |
| 7-25 (R) | 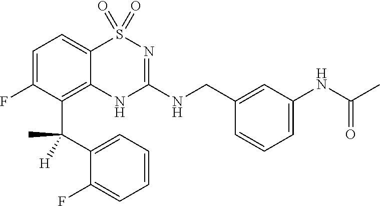 [SMILES: CC(=O)Nc1cccc(CNC2=NS(=O)(=O)c3ccc(F)c([C@H](C)c4ccccc4F)c3N2)c1] | (R)-N-(3-(((6-fluoro-5-(1-(2- fluorophenyl)ethyl)-1,1-dioxido- 4H-benzo[e][1,2,4]thiadiazin-3- yl)amino)methyl)phenyl)acetamide |
| 7-26 | 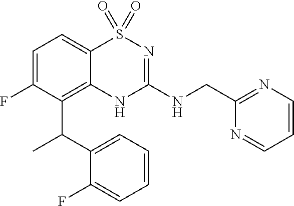 [SMILES: CC(c1ccccc1F)c1c(F)ccc2c1NC(NCc1ncccn1)=NS2(=O)=O] | 6-fluoro-5-(1-(2- fluorophenyl)ethyl)-3-((pyrimidin- 2-ylmethyl)amino)-4H- benzo[e][1,2,4]thiadiazine 1,1- dioxide |
| 7-26 (S) | 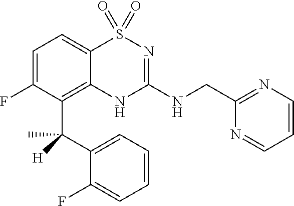 [SMILES: C[C@@H](c1ccccc1F)c1c(F)ccc2c1NC(NCc1ncccn1)=NS2(=O)=O] | (S)-6-fluoro-5-(1-(2- fluorophenyl)ethyl)-3-((pyrimidin- 2-ylmethyl)amino)-4H- benzo[e][1,2,4]thiadiazine 1,1- dioxide |
| 7-26 (R) | 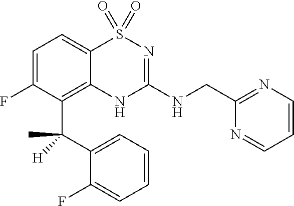 [SMILES: C[C@H](c1ccccc1F)c1c(F)ccc2c1NC(NCc1ncccn1)=NS2(=O)=O] | (R)-6-fluoro-5-(1-(2- fluorophenyl)ethyl)-3-((pyrimidin- 2-ylmethyl)amino)-4H- benzo[e][1,2,4]thiadiazine 1,1- dioxide |
| 7-27 | 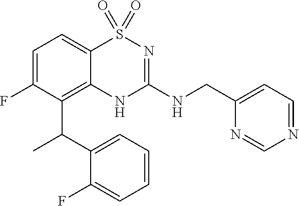 [SMILES: CC(c1ccccc1F)c1c(F)ccc2c1NC(NCc1ccncn1)=NS2(=O)=O] | 6-fluoro-5-(1-(2- fluorophenyl)ethyl)-3-((pyrimidin- 4-ylmethyl)amino)-4H- benzo[e][1,2,4]thiadiazine 1,1- dioxide |
| 7-27 (S) | 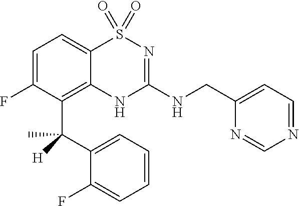 [SMILES: C[C@@H](c1ccccc1F)c1c(F)ccc2c1NC(NCc1ccncn1)=NS2(=O)=O] | (S)-6-fluoro-5-(1-(2- fluorophenyl)ethyl)-3-((pyrimidin- 4-ylmethyl)amino)-4H- benzo[e][1,2,4]thiadiazine 1,1- dioxide |
| 7-27 (R) | 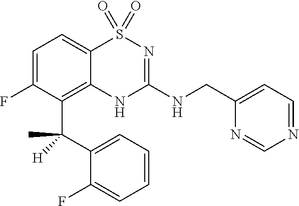 [SMILES: C[C@H](c1ccccc1F)c1c(F)ccc2c1NC(NCc1ccncn1)=NS2(=O)=O] | (R)-6-fluoro-5-(1-(2- fluorophenyl)ethyl)-3-((pyrimidin- 4-ylmethyl)amino)-4H- benzo[e][1,2,4]thiadiazine 1,1- dioxide |
| 7-28 | 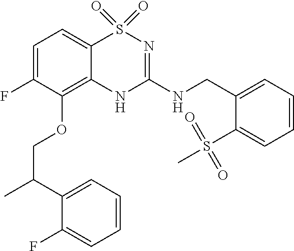 [SMILES: CC(COc1c(F)ccc2c1NC(NCc1ccccc1S(C)(=O)=O)=NS2(=O)=O)c1ccccc1F] | 6-fluoro-5-(1-(2- fluorophenyl)ethyl)-3-((2- (methylsulfonyl)benzyl)amino)- 4H-benzo[e][1,2,4]thiadiazine 1,1- dioxide |
| 7-28 (S) | 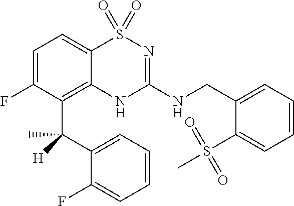 [SMILES: C[C@@H](c1ccccc1F)c1c(F)ccc2c1NC(NCc1ccccc1S(C)(=O)=O)=NS2(=O)=O] | (S)-6-fluoro-5-(1-(2- fluorophenyl)ethyl)-3-((2- (methylsulfonyl)benzyl)amino)- 4H-benzo[e][1,2,4]thiadiazine 1,1- dioxide |
| 7-28 (R) | 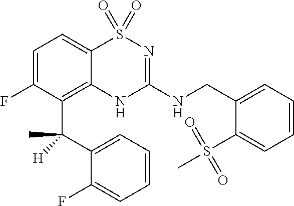 [SMILES: C[C@H](c1ccccc1F)c1c(F)ccc2c1NC(NCc1ccccc1S(C)(=O)=O)=NS2(=O)=O] | (R)-6-fluoro-5-(1-(2- fluorophenyl)ethyl)-3-((2- (methylsulfonyl)benzyl)amino)- 4H-benzo[e][1,2,4]thiadiazine 1,1- dioxide |
| 7-29 | 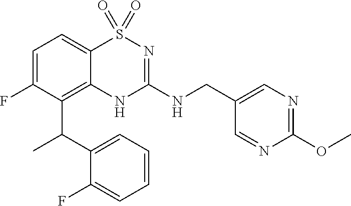 [SMILES: COc1ncc(CNC2=NS(=O)(=O)c3ccc(F)c(C(C)c4ccccc4F)c3N2)cn1] | 6-fluoro-5-(1-(2- fluorophenyl)ethyl)-3-(((2- methoxypyrimidin-5- yl)methyl)amino)-4H- benzo[e][1,2,4]thiadiazine 1,1- dioxide |
| 7-29 (S) | 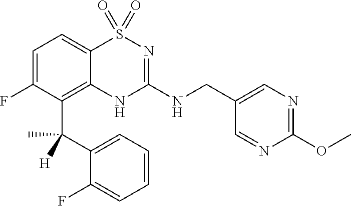 [SMILES: COc1ncc(CNC2=NS(=O)(=O)c3ccc(F)c([C@@H](C)c4ccccc4F)c3N2)cn1] | (S)-6-fluoro-5-(1-(2- fluorophenyl)ethyl)-3-(((2- methoxypyrimidin-5- yl)methyl)amino)-4H- benzo[e][1,2,4]thiadiazine 1,1- dioxide |
| 7-29 (R) | 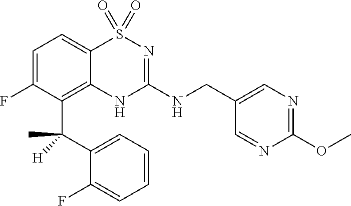 [SMILES: COc1ncc(CNC2=NS(=O)(=O)c3ccc(F)c([C@H](C)c4ccccc4F)c3N2)cn1] | (R)-6-fluoro-5-(1-(2- fluorophenyl)ethyl)-3-(((2- methoxypyrimidin-5- yl)methyl)amino)-4H- benzo[e][1,2,4]thiadiazine 1,1- dioxide |
| 7-30 | 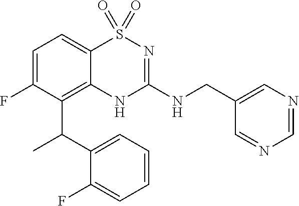 [SMILES: CC(c1ccccc1F)c1c(F)ccc2c1NC(NCc1cncnc1)=NS2(=O)=O] | 6-fluoro-5-(1-(2- fluorophenyl)ethyl)-3-((pyrimidin- 5-ylmethyl)amino)-4H- benzo[e][1,2,4]thiadiazine 1,1- dioxide |
| 7-30 (S) | 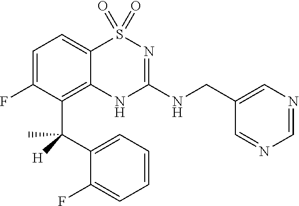 [SMILES: C[C@@H](c1ccccc1F)c1c(F)ccc2c1NC(NCc1cncnc1)=NS2(=O)=O] | (S)-6-fluoro-5-(1-(2- fluorophenyl)ethyl)-3-((pyrimidin- 5-ylmethyl)amino)-4H- benzo[e][1,2,4]thiadiazine 1,1- dioxide |
| 7-30 (R) | 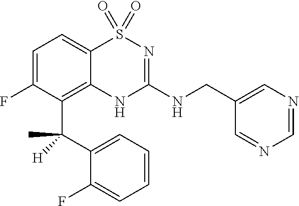 [SMILES: C[C@H](c1ccccc1F)c1c(F)ccc2c1NC(NCc1cncnc1)=NS2(=O)=O] | (R)-6-fluoro-5-(1-(2- fluorophenyl)ethyl)-3-((pyrimidin- 5-ylmethyl)amino)-4H- benzo[e][1,2,4]thiadiazine 1,1- dioxide |
| 7-31 | 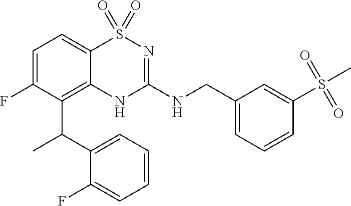 [SMILES: CC(c1ccccc1F)c1c(F)ccc2c1NC(NCc1cccc(S(C)(=O)=O)c1)=NS2(=O)=O] | 6-fluoro-5-(1-(2- fluorophenyl)ethyl)-3-((3- (methylsulfonyl)benzyl)amino)- 4H-benzo[e][1,2,4]thiadiazine 1,1- dioxide |
| 7-31 (S) | 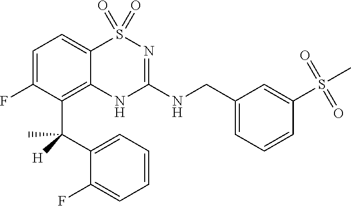 [SMILES: C[C@@H](c1ccccc1F)c1c(F)ccc2c1NC(NCc1cccc(S(C)(=O)=O)c1)=NS2(=O)=O] | (S)-6-fluoro-5-(1-(2- fluorophenyl)ethyl)-3-((3- (methylsulfonyl)benzyl)amino)- 4H-benzo[e][1,2,4]thiadiazine 1,1- dioxide |
| 7-31 (R) | 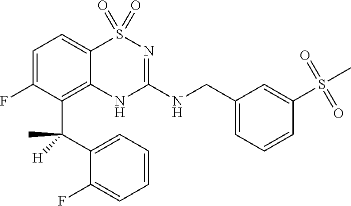 [SMILES: C[C@H](c1ccccc1F)c1c(F)ccc2c1NC(NCc1cccc(S(C)(=O)=O)c1)=NS2(=O)=O] | (R)-6-fluoro-5-(1-(2- fluorophenyl)ethyl)-3-((3- (methylsulfonyl)benzyl)amino)- 4H-benzo[e][1,2,4]thiadiazine 1,1- dioxide |
| 7-32 | 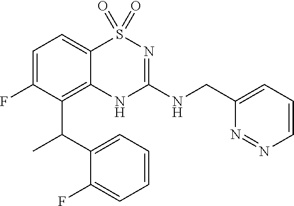 [SMILES: CC(c1ccccc1F)c1c(F)ccc2c1NC(NCc1cccnn1)=NS2(=O)=O] | 6-fluoro-5-(1-(2- fluorophenyl)ethyl)-3-((pyridazin- 3-ylmethyl)amino)-4H- benzo[e][1,2,4]thiadiazine 1,1- dioxide |
| 7-32 (S) | 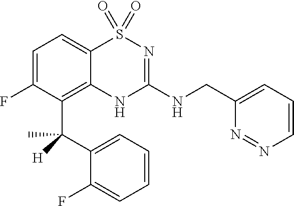 [SMILES: C[C@@H](c1ccccc1F)c1c(F)ccc2c1NC(NCc1cccnn1)=NS2(=O)=O] | (S)-6-fluoro-5-(1-(2- fluorophenyl)ethyl)-3-((pyridazin- 3-ylmethyl)amino)-4H- benzo[e][1,2,4]thiadiazine 1,1- dioxide |
| 7-32 (R) | 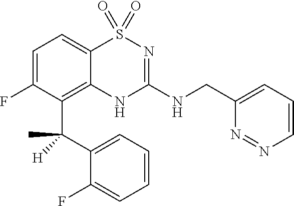 [SMILES: C[C@H](c1ccccc1F)c1c(F)ccc2c1NC(NCc1cccnn1)=NS2(=O)=O] | (R)-6-fluoro-5-(1-(2- fluorophenyl)ethyl)-3-((pyridazin- 3-ylmethyl)amino)-4H- benzo[e][1,2,4]thiadiazine 1,1- dioxide |
| 7-33 | 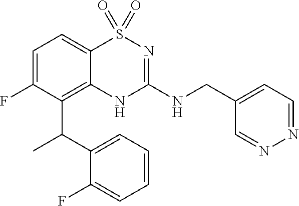 [SMILES: CC(c1ccccc1F)c1c(F)ccc2c1NC(NCc1ccnnc1)=NS2(=O)=O] | 6-fluoro-5-(1-(2- fluorophenyl)ethyl)-3-((pyridazin- 4-ylmethyl)amino)-4H- benzo[e][1,2,4]thiadiazine 1,1- dioxide |
| 7-33 (S) | 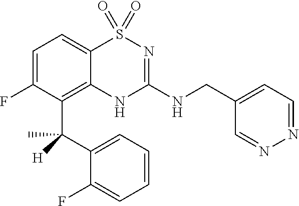 [SMILES: C[C@@H](c1ccccc1F)c1c(F)ccc2c1NC(NCc1ccnnc1)=NS2(=O)=O] | (S)- 6-fluoro-5-(1-(2- fluorophenyl)ethyl)-3-((pyridazin- 4-ylmethyl)amino)-4H- benzo[e][1,2,4]thiadiazine 1,1- dioxide |
| 7-33 (R) | 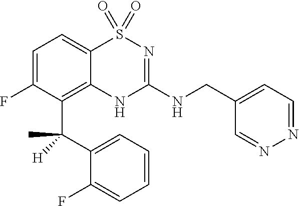 [SMILES: C[C@H](c1ccccc1F)c1c(F)ccc2c1NC(NCc1ccnnc1)=NS2(=O)=O] | (R)-6-fluoro-5-(1-(2- fluorophenyl)ethyl)-3-((pyridazin- 4-ylmethyl)amino)-4H- benzo[e][1,2,4]thiadiazine 1,1- dioxide |
| 7-34 | 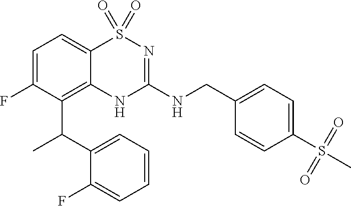 [SMILES: CC(c1ccccc1F)c1c(F)ccc2c1NC(NCc1ccc(S(C)(=O)=O)cc1)=NS2(=O)=O] | 6-fluoro-5-(1-(2- fluorophenyl)ethyl)-3-((4- (methylsulfonyl)benzyl)amino)- 4H-benzo[e][1,2,4]thiadiazine 1,1- dioxide |
| 7-34 (S) | 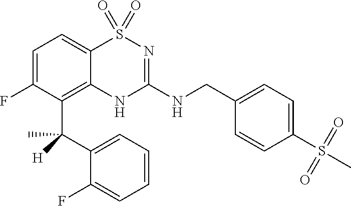 [SMILES: C[C@@H](c1ccccc1F)c1c(F)ccc2c1NC(NCc1ccc(S(C)(=O)=O)cc1)=NS2(=O)=O] | (S)-6-fluoro-5-(1-(2- fluorophenyl)ethyl)-3-((4- (methylsulfonyl)benzyl)amino)- 4H-benzo[e][1,2,4]thiadiazine 1,1- dioxide |
| 7-34 (R) | 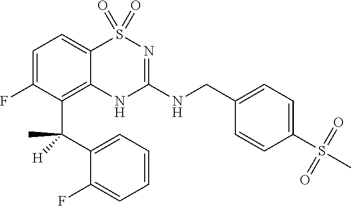 [SMILES: C[C@H](c1ccccc1F)c1c(F)ccc2c1NC(NCc1ccc(S(C)(=O)=O)cc1)=NS2(=O)=O] | (R)-6-fluoro-5-(1-(2- fluorophenyl)ethyl)-3-((4- (methylsulfonyl)benzyl)amino)- 4H-benzo[e][1,2,4]thiadiazine 1,1- dioxide |
| 7-35 | 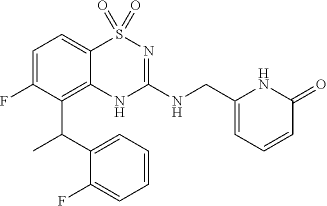 [SMILES: CC(c1ccccc1F)c1c(F)ccc2c1NC(NCc1cccc(=O)[nH]1)=NS2(=O)=O] | 6-(((6-fluoro-5-(1-(2- fluorophenyl)ethyl)-1,1-dioxido- 4H-benzo[e][1,2,4]thiadiazin-3- yl)amino)methyl)pyridin-2(1H)- one |
| 7-35 (S) | 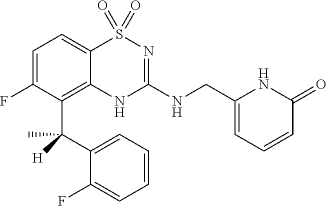 [SMILES: C[C@@H](c1ccccc1F)c1c(F)ccc2c1NC(NCc1cccc(=O)[nH]1)=NS2(=O)=O] | (S)-6-(((6-fluoro-5-(1-(2- fluorophenyl)ethyl)-1,1-dioxido- 4H-benzo[e][1,2,4]thiadiazin-3- yl)amino)methyl)pyridin-2(1H)- one |
| 7-35 (R) | 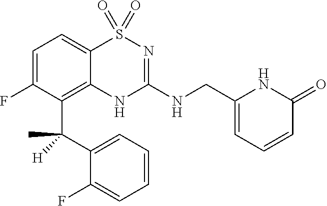 [SMILES: C[C@H](c1ccccc1F)c1c(F)ccc2c1NC(NCc1cccc(=O)[nH]1)=NS2(=O)=O] | (R)-6-(((6-fluoro-5-(1-(2- fluorophenyl)ethyl)-1,1-dioxido- 4H-benzo[e][1,2,4]thiadiazin-3- yl)amino)methyl)pyridin-2(1H)- one |
| 7-36 | 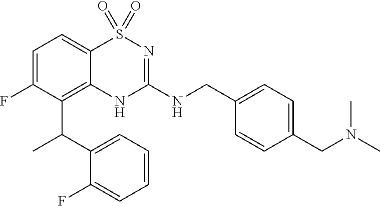 [SMILES: CC(c1ccccc1F)c1c(F)ccc2c1NC(NCc1ccc(CN(C)C)cc1)=NS2(=O)=O] | 3-((4- ((dimethylamino)methyl)benzyl) amino)-6-fluoro-5-(1-(2- fluorophenyl)ethyl)-4H- benzo[e][1,2,4]thiadiazine 1,1- dioxide |
| 7-36 (S) | 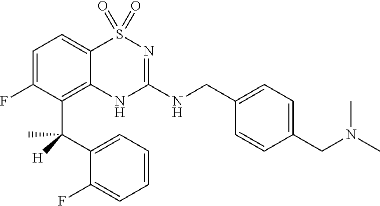 [SMILES: C[C@@H](c1ccccc1F)c1c(F)ccc2c1NC(NCc1ccc(CN(C)C)cc1)=NS2(=O)=O] | (S)-3-((4- ((dimethylamino)methyl)benzyl) amino)-6-fluoro-5-(1-(2- fluorophenyl)ethyl)-4H- benzo[e][1,2,4]thiadiazine 1,1- dioxide |
| 7-36 (R) | 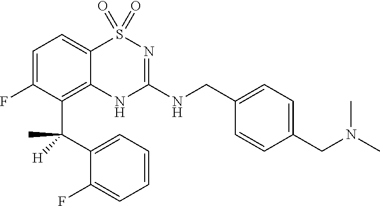 [SMILES: C[C@H](c1ccccc1F)c1c(F)ccc2c1NC(NCc1ccc(CN(C)C)cc1)=NS2(=O)=O] | (R)-3-((4- ((dimethylamino)methyl)benzyl) amino)-6-fluoro-5-(1-(2- fluorophenyl)ethyl)-4H- benzo[e][1,2,4]thiadiazine 1,1- dioxide |
| 7-37 | 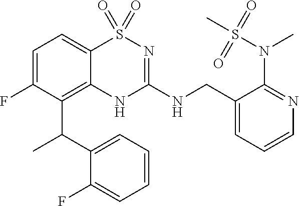 [SMILES: CC(c1ccccc1F)c1c(F)ccc2c1NC(NCc1cccnc1N(C)S(C)(=O)=O)=NS2(=O)=O] | N-(3-(((6-fluoro-5-(1-(2- fluorophenyl)ethyl)-1,1-dioxido- 4H-benzo[e][1,2,4]thiadiazin-3- yl)amino)methyl)pyridin-2-yl)-N- methylmethanesulfonamide |
| 7-37 (S) | 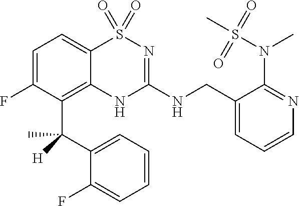 [SMILES: C[C@@H](c1ccccc1F)c1c(F)ccc2c1NC(NCc1cccnc1N(C)S(C)(=O)=O)=NS2(=O)=O] | (S)-N-(3-(((6-fluoro-5-(1-(2- fluorophenyl)ethyl)-1,1-dioxido- 4H-benzo[e][1,2,4]thiadiazin-3- yl)amino)methyl)pyridin-2-yl)-N- methylmethanesulfonamide |
| 7-37 (R) | 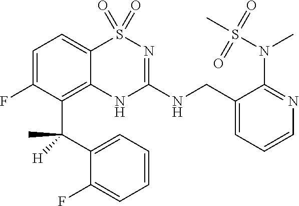 [SMILES: C[C@H](c1ccccc1F)c1c(F)ccc2c1NC(NCc1cccnc1N(C)S(C)(=O)=O)=NS2(=O)=O] | (R)-N-(3-(((6-fluoro-5-(1-(2- fluorophenyl)ethyl)-1,1-dioxido- 4H-benzo[e][1,2,4]thiadiazin-3- yl)amino)methyl)pyridin-2-yl)-N- methylmethanesulfonamide |
| 7-38 | 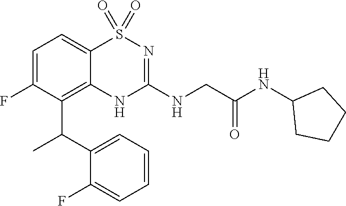 [SMILES: CC(c1ccccc1F)c1c(F)ccc2c1NC(NCC(=O)NC1CCCC1)=NS2(=O)=O] | N-cyclopentyl-2-((6-fluoro-5-(1- (2-fluorophenyl)ethyl)-1,1- dioxido-4H- benzo[e][1,2,4]thiadiazin-3- yl)amino)acetamide |
| 7-38 (S) | 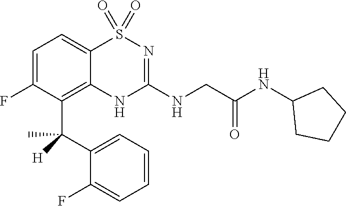 [SMILES: C[C@@H](c1ccccc1F)c1c(F)ccc2c1NC(NCC(=O)NC1CCCC1)=NS2(=O)=O] | (S)-N-cyclopentyl-2-((6-fluoro-5- (1-(2-fluorophenyl)ethyl)-1,1- dioxido-4H- benzo[e][1,2,4]thiadiazin-3- yl)amino)acetamide |
| 7-38 (R) | 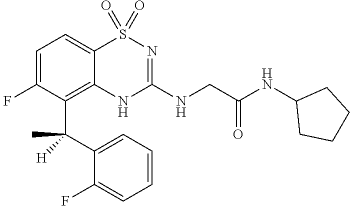 [SMILES: C[C@H](c1ccccc1F)c1c(F)ccc2c1NC(NCC(=O)NC1CCCC1)=NS2(=O)=O] | (R)-N-cyclopentyl-2-((6-fluoro-5- (1-(2-fluorophenyl)ethyl)-1,1- dioxido-4H- benzo[e][1,2,4]thiadiazin-3- yl)amino)acetamide |
| 7-39 | 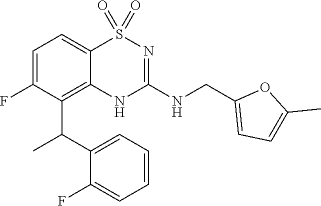 [SMILES: Cc1ccc(CNC2=NS(=O)(=O)c3ccc(F)c(C(C)c4ccccc4F)c3N2)o1] | 6-fluoro-5-(1-(2- fluorophenyl)ethyl)-3-(((5- methylfuran-2-yl)methyl)amino)- 4H-benzo[e][1,2,4]thiadiazine 1,1- dioxide |
| 7-39 (S) | 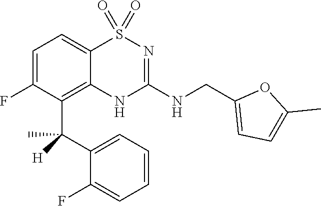 [SMILES: Cc1ccc(CNC2=NS(=O)(=O)c3ccc(F)c([C@@H](C)c4ccccc4F)c3N2)o1] | (S)-6-fluoro-5-(1-(2- fluorophenyl)ethyl)-3-(((5- methylfuran-2-yl)methyl)amino)- 4H-benzo[e][1,2,4]thiadiazine 1,1- dioxide |
| 7-39 (R) | 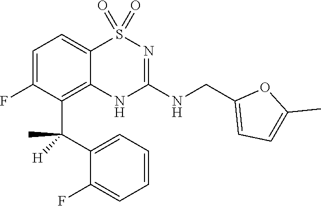 [SMILES: Cc1ccc(CNC2=NS(=O)(=O)c3ccc(F)c([C@H](C)c4ccccc4F)c3N2)o1] | (R)-6-fluoro-5-(1-(2- fluorophenyl)ethyl)-3-(((5- methylfuran-2-yl)methyl)amino)- 4H-benzo[e][1,2,4]thiadiazine 1,1- dioxide |
| 7-40 | 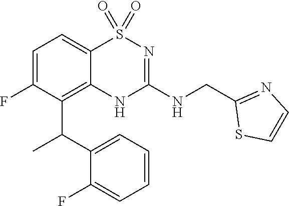 [SMILES: CC(c1ccccc1F)c1c(F)ccc2c1NC(NCc1nccs1)=NS2(=O)=O] | 6-fluoro-5-(1-(2- fluorophenyl)ethyl)-3-((thiazol-2- ylmethyl)amino)-4H- benzo[e][1,2,4]thiadiazine 1,1- dioxide |
| 7-40 (S) | 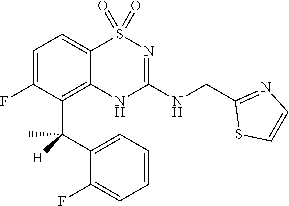 [SMILES: C[C@@H](c1ccccc1F)c1c(F)ccc2c1NC(NCc1nccs1)=NS2(=O)=O] | (S)-6-fluoro-5-(1-(2- fluorophenyl)ethyl)-3-((thiazol-2- ylmethyl)amino)-4H- benzo[e][1,2,4]thiadiazine 1,1- dioxide |
| 7-40 (R) | 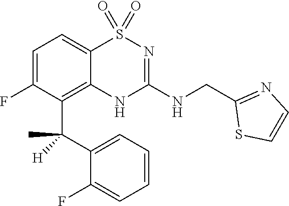 [SMILES: C[C@H](c1ccccc1F)c1c(F)ccc2c1NC(NCc1nccs1)=NS2(=O)=O] | (R)-6-fluoro-5-(1-(2- fluorophenyl)ethyl)-3-((thiazol-2- ylmethyl)amino)-4H- benzo[e][1,2,4]thiadiazine 1,1- dioxide |
| 7-41 | 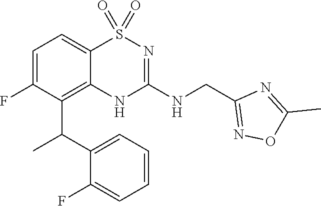 [SMILES: Cc1nc(CNC2=NS(=O)(=O)c3ccc(F)c(C(C)c4ccccc4F)c3N2)no1] | 6-fluoro-5-(1-(2- fluorophenyl)ethyl)-3-(((5-methyl- 1,2,4-oxadiazol-3- yl)methyl)amino)-4H- benzo[e][1,2,4]thiadiazine 1,1- dioxide |
| 7-41 (S) | 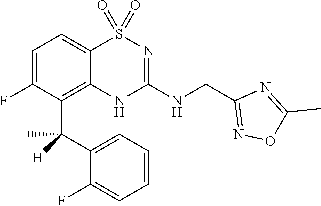 [SMILES: Cc1nc(CNC2=NS(=O)(=O)c3ccc(F)c([C@@H](C)c4ccccc4F)c3N2)no1] | (S)-6-fluoro-5-(1-(2- fluorophenyl)ethyl)-3-(((5-methyl- 1,2,4-oxadiazol-3- yl)methyl)amino)-4H- benzo[e][1,2,4]thiadiazine 1,1- dioxide |
| 7-41 (R) | 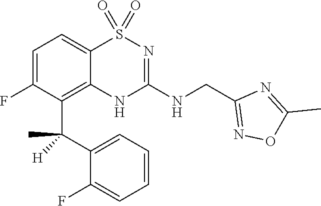 [SMILES: Cc1nc(CNC2=NS(=O)(=O)c3ccc(F)c([C@H](C)c4ccccc4F)c3N2)no1] | (R)-6-fluoro-5-(1-(2- fluorophenyl)ethyl)-3-(((5-methyl- 1,2,4-oxadiazol-3- yl)methyl)amino)-4H- benzo[e][1,2,4]thiadiazine 1,1- dioxide |
| 7-42 | 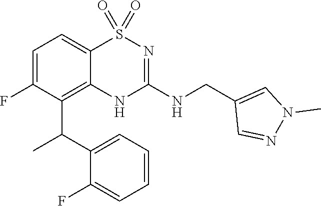 [SMILES: CC(c1ccccc1F)c1c(F)ccc2c1NC(NCc1cnn(C)c1)=NS2(=O)=O] | 6-fluoro-5-(1-(2- fluorophenyl)ethyl)-3-(((1-methyl- 1H-pyrazol-4-yl)methyl)amino)- 4H-benzo[e][1,2,4]thiadiazine 1,1- dioxide |
| 7-42 (S) | 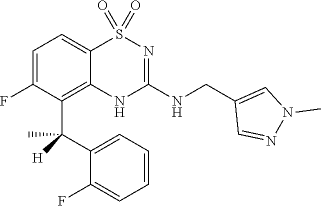 [SMILES: C[C@@H](c1ccccc1F)c1c(F)ccc2c1NC(NCc1cnn(C)c1)=NS2(=O)=O] | (S)-6-fluoro-5-(1-(2- fluorophenyl)ethyl)-3-(((1-methyl- 1H-pyrazol-4-yl)methyl)amino)- 4H-benzo[e][1,2,4]thiadiazine 1,1- dioxide |
| 7-42 (R) | 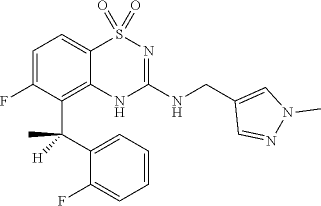 [SMILES: C[C@H](c1ccccc1F)c1c(F)ccc2c1NC(NCc1cnn(C)c1)=NS2(=O)=O] | (R)-6-fluoro-5-(1-(2- fluorophenyl)ethyl)-3-(((1-methyl- 1H-pyrazol-4-yl)methyl)amino)- 4H-benzo[e][1,2,4]thiadiazine 1,1- dioxide |
| 7-43 | 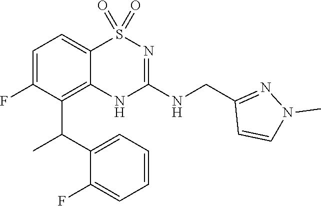 [SMILES: CC(c1ccccc1F)c1c(F)ccc2c1NC(NCc1ccn(C)n1)=NS2(=O)=O] | 6-fluoro-5-(1-(2- fluorophenyl)ethyl)-3-(((1-methyl- 1H-pyrazol-3-yl)methyl)amino)- 4H-benzo[e][1,2,4]thiadiazine 1,1- dioxide |
| 7-43 (S) | 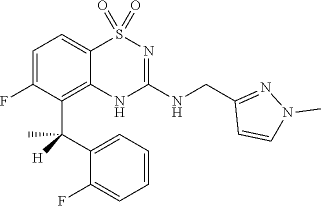 [SMILES: C[C@@H](c1ccccc1F)c1c(F)ccc2c1NC(NCc1ccn(C)n1)=NS2(=O)=O] | (S)-6-fluoro-5-(1-(2- fluorophenyl)ethyl)-3-(((1-methyl- 1H-pyrazol-3-yl)methyl)amino)- 4H-benzo[e][1,2,4]thiadiazine 1,1- dioxide |
| 7-43 (R) | 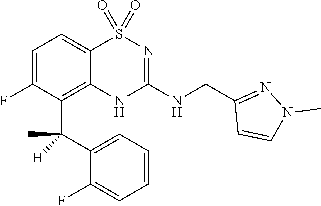 [SMILES: C[C@H](c1ccccc1F)c1c(F)ccc2c1NC(NCc1ccn(C)n1)=NS2(=O)=O] | (R)-6-fluoro-5-(1-(2- fluorophenyl)ethyl)-3-(((1-methyl- 1H-pyrazol-3-yl)methyl)amino)- 4H-benzo[e][1,2,4]thiadiazine 1,1- dioxide |
| 7-44 | 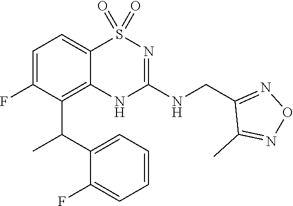 [SMILES: Cc1nonc1CNC1=NS(=O)(=O)c2ccc(F)c(C(C)c3ccccc3F)c2N1] | 6-fluoro-5-(1-(2- fluorophenyl)ethyl)-3-(((4-methyl- 1,2,5-oxadiazol-3- yl)methyl)amino)-4H- benzo[e][1,2,4]thiadiazine 1,1- dioxide |
| 7-44 (S) | 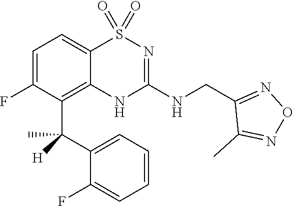 [SMILES: Cc1nonc1CNC1=NS(=O)(=O)c2ccc(F)c([C@@H](C)c3ccccc3F)c2N1] | (S)-6-fluoro-5-(1-(2- fluorophenyl)ethyl)-3-(((4-methyl- 1,2,5-oxadiazol-3- yl)methyl)amino)-4H- benzo[e][1,2,4]thiadiazine 1,1- dioxide |
| 7-44 (R) | 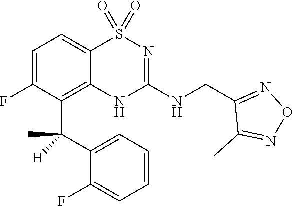 [SMILES: Cc1nonc1CNC1=NS(=O)(=O)c2ccc(F)c([C@H](C)c3ccccc3F)c2N1] | (R)-6-fluoro-5-(1-(2- fluorophenyl)ethyl)-3-(((4-methyl- 1,2,5-oxadiazol-3- yl)methyl)amino)-4H- benzo[e][1,2,4]thiadiazine 1,1- dioxide |
| 7-45 | 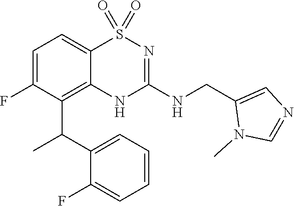 [SMILES: CC(c1ccccc1F)c1c(F)ccc2c1NC(NCc1cncn1C)=NS2(=O)=O] | 6-fluoro-5-(1-(2- fluorophenyl)ethyl)-3-(((1-methyl- 1H-imidazol-5-yl)methyl)amino)- 4H-benzo[e][1,2,4]thiadiazine 1,1- dioxide |
| 7-45 (S) | 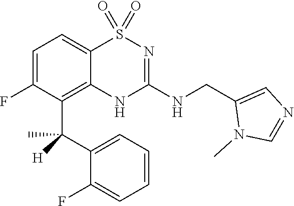 [SMILES: C[C@@H](c1ccccc1F)c1c(F)ccc2c1NC(NCc1cncn1C)=NS2(=O)=O] | (S)-6-fluoro-5-(1-(2- fluorophenyl)ethyl)-3-(((1-methyl- 1H-imidazol-5-yl)methyl)amino)- 4H-benzo[e][1,2,4]thiadiazine 1,1- dioxide |
| 7-45 (R) | 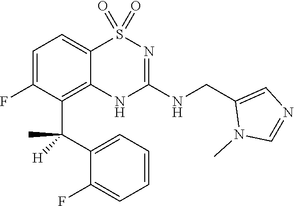 [SMILES: C[C@H](c1ccccc1F)c1c(F)ccc2c1NC(NCc1cncn1C)=NS2(=O)=O] | (R)-6-fluoro-5-(1-(2- fluorophenyl)ethyl)-3-(((1-methyl- 1H-imidazol-5-yl)methyl)amino)- 4H-benzo[e][1,2,4]thiadiazine 1,1- dioxide |
| 7-46 | 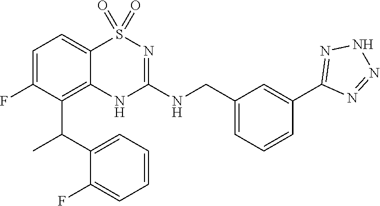 [SMILES: CC(c1ccccc1F)c1c(F)ccc2c1NC(NCc1cccc(-c3nn[nH]n3)c1)=NS2(=O)=O] | 3-((3-(2H-tetrazol-5- yl)benzyl)amino)-6-fluoro-5-(1-(2- fluorophenyl)ethyl)-4H- benzo[e][1,2,4]thiadiazine 1,1- dioxide |
| 7-46 (S) | 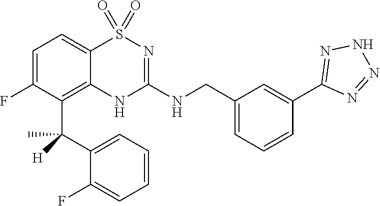 [SMILES: C[C@@H](c1ccccc1F)c1c(F)ccc2c1NC(NCc1cccc(-c3nn[nH]n3)c1)=NS2(=O)=O] | (S)-3-((3-(2H-tetrazol-5- yl)benzyl)amino)-6-fluoro-5-(1-(2- fluorophenyl)ethyl)-4H- benzo[e][1,2,4]thiadiazine 1,1- dioxide |
| 7-46 (R) | 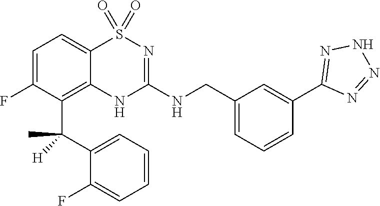 [SMILES: C[C@H](c1ccccc1F)c1c(F)ccc2c1NC(NCc1cccc(-c3nn[nH]n3)c1)=NS2(=O)=O] | (R)-3-((3-(2H-tetrazol-5- yl)benzyl)amino)-6-fluoro-5-(1-(2- fluorophenyl)ethyl)-4H- benzo[e][1,2,4]thiadiazine 1,1- dioxide |
| 7-47 | 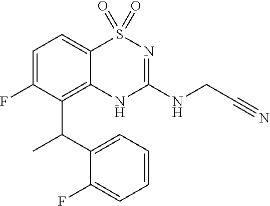 [SMILES: CC(c1ccccc1F)c1c(F)ccc2c1NC(NCC#N)=NS2(=O)=O] | 2-((6-fluoro-5-(1-(2- fluorophenyl)ethyl)-1,1-dioxido- 4H-benzo[e][1,2,4]thiadiazin-3- yl)amino)acetonitrile |
| 7-47 (S) | 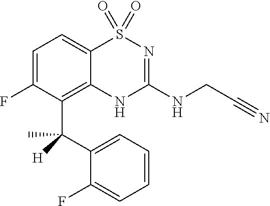 [SMILES: C[C@@H](c1ccccc1F)c1c(F)ccc2c1NC(NCC#N)=NS2(=O)=O] | (S)-2-((6-fluoro-5-(1-(2- fluorophenyl)ethyl)-1,1-dioxido- 4H-benzo[e][1,2,4]thiadiazin-3- yl)amino)acetonitrile |
| 7-47 (R) | 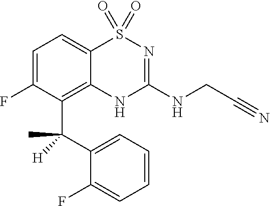 [SMILES: C[C@H](c1ccccc1F)c1c(F)ccc2c1NC(NCC#N)=NS2(=O)=O] | (R)-2-((6-fluoro-5-(1-(2- fluorophenyl)ethyl)-1,1-dioxido- 4H-benzo[e][1,2,4]thiadiazin-3- yl)amino)acetonitrile |
| 7-48 | 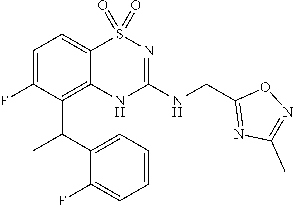 [SMILES: Cc1noc(CNC2=NS(=O)(=O)c3ccc(F)c(C(C)c4ccccc4F)c3N2)n1] | 6-fluoro-5-(1-(2- fluorophenyl)ethyl)-3-(((3-methyl- 1,2,4-oxadiazol-5- yl)methyl)amino)-4H- benzo[e][1,2,4]thiadiazine 1,1- dioxide |
| 7-48 (S) | 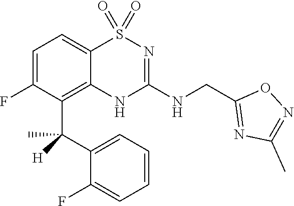 [SMILES: Cc1noc(CNC2=NS(=O)(=O)c3ccc(F)c([C@@H](C)c4ccccc4F)c3N2)n1] | (S)-6-fluoro-5-(1-(2- fluorophenyl)ethyl)-3-(((3-methyl- 1,2,4-oxadiazol-5- yl)methyl)amino)-4H- benzo[e][1,2,4]thiadiazine 1,1- dioxide |
| 7-48 (R) | 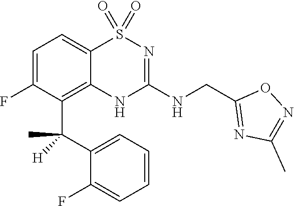 [SMILES: Cc1noc(CNC2=NS(=O)(=O)c3ccc(F)c([C@H](C)c4ccccc4F)c3N2)n1] | (R)-6-fluoro-5-(1-(2- fluorophenyl)ethyl)-3-(((3-methyl- 1,2,4-oxadiazol-5- yl)methyl)amino)-4H- benzo[e][1,2,4]thiadiazine 1,1- dioxide |
| 7-49 | 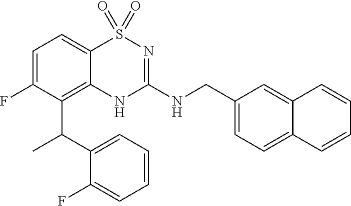 [SMILES: CC(c1ccccc1F)c1c(F)ccc2c1NC(NCc1ccc3ccccc3c1)=NS2(=O)=O] | 6-fluoro-5-(1-(2- fluorophenyl)ethyl)-3- ((naphthalen-2-ylmethyl)amino)- 4H-benzo[e][1,2,4]thiadiazine 1,1- dioxide |
| 7-49 (S) | 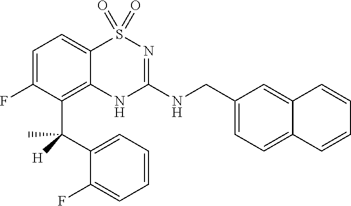 [SMILES: C[C@@H](c1ccccc1F)c1c(F)ccc2c1NC(NCc1ccc3ccccc3c1)=NS2(=O)=O] | (S)-6-fluoro-5-(1-(2- fluorophenyl)ethyl)-3- ((naphthalen-2-ylmethyl)amino)- 4H-benzo[e][1,2,4]thiadiazine 1,1- dioxide |
| 7-49 (R) | 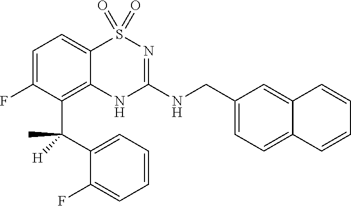 [SMILES: C[C@H](c1ccccc1F)c1c(F)ccc2c1NC(NCc1ccc3ccccc3c1)=NS2(=O)=O] | (R)-6-fluoro-5-(1-(2- fluorophenyl)ethyl)-3- ((naphthalen-2-ylmethyl)amino)- 4H-benzo[e][1,2,4]thiadiazine 1,1- dioxide |
| 7-50 | 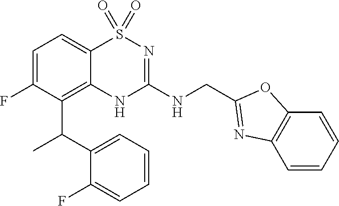 [SMILES: CC(c1ccccc1F)c1c(F)ccc2c1NC(NCc1nc3ccccc3o1)=NS2(=O)=O] | 3-((benzo[d]oxazol-2- ylmethyl)amino)-6-fluoro-5-(1-(2- fluorophenyl)ethyl)-4H- benzo[e][1,2,4]thiadiazine 1,1- dioxide |
| 7-50 (S) | 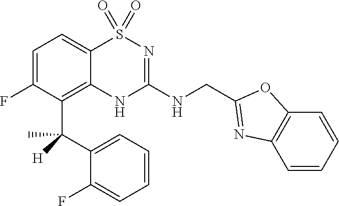 [SMILES: C[C@@H](c1ccccc1F)c1c(F)ccc2c1NC(NCc1nc3ccccc3o1)=NS2(=O)=O] | (S)-3-((benzo[d]oxazol-2- ylmethyl)amino)-6-fluoro-5-(1-(2- fluorophenyl)ethyl)-4H- benzo[e][1,2,4]thiadiazine 1,1- dioxide |
| 7-50 (R) | 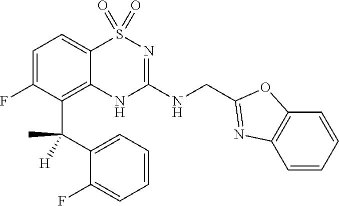 [SMILES: C[C@H](c1ccccc1F)c1c(F)ccc2c1NC(NCc1nc3ccccc3o1)=NS2(=O)=O] | (R)-3-((benzo[d]oxazol-2- ylmethyl)amino)-6-fluoro-5-(1-(2- fluorophenyl)ethyl)-4H- benzo[e][1,2,4]thiadiazine 1,1- dioxide |
| 7-51 | 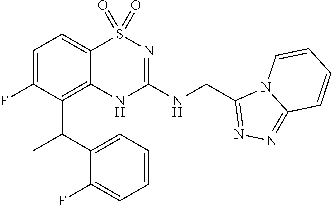 [SMILES: CC(c1ccccc1F)c1c(F)ccc2c1NC(NCc1nnc3ccccn13)=NS2(=O)=O] | 3-(([1,2,4]triazolo[4,3-a]pyridin-3- ylmethyl)amino)-6-fluoro-5-(1-(2- fluorophenyl)ethyl)-4H- benzo[e][1,2,4]thiadiazine 1,1- dioxide |
| 7-51 (S) | 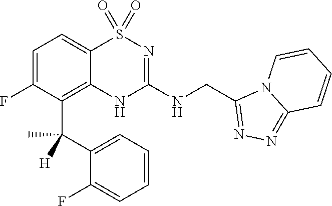 [SMILES: C[C@@H](c1ccccc1F)c1c(F)ccc2c1NC(NCc1nnc3ccccn13)=NS2(=O)=O] | (S)-3-((benzo[d]oxazol-2- ylmethyl)amino)-6-fluoro-5-(1-(2- fluorophenyl)ethyl)-4H- benzo[e][1,2,4]thiadiazine 1,1- dioxide |
| 7-51 (R) | 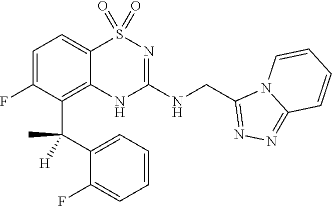 [SMILES: C[C@H](c1ccccc1F)c1c(F)ccc2c1NC(NCc1nnc3ccccn13)=NS2(=O)=O] | (R)-3-((benzo[d]oxazol-2- ylmethyl)amino)-6-fluoro-5-(1-(2- fluorophenyl)ethyl)-4H- benzo[e][1,2,4]thiadiazine 1,1- dioxide |
| 7-52 | 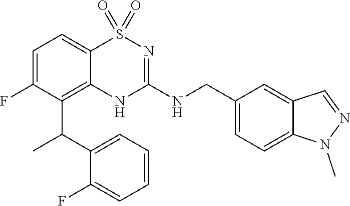 [SMILES: CC(c1ccccc1F)c1c(F)ccc2c1NC(NCc1ccc3c(cnn3C)c1)=NS2(=O)=O] | 6-fluoro-5-(1-(2- fluorophenyl)ethyl)-3-(((1-methyl- 1H-indazol-5-yl)methyl)amino)- 4H-benzo[e][1,2,4]thiadiazine 1,1- dioxide |
| 7-52 (S) | 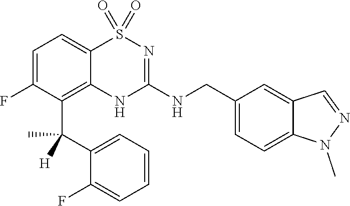 [SMILES: C[C@@H](c1ccccc1F)c1c(F)ccc2c1NC(NCc1ccc3c(cnn3C)c1)=NS2(=O)=O] | (S)-6-fluoro-5-(1-(2- fluorophenyl)ethyl)-3-(((1-methyl- 1H-indazol-5-yl)methyl)amino)- 4H-benzo[e][1,2,4]thiadiazine 1,1- dioxide |
| 7-52 (R) | 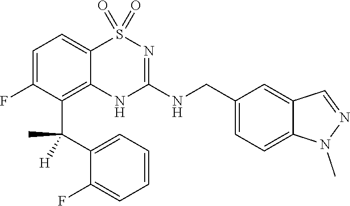 [SMILES: C[C@H](c1ccccc1F)c1c(F)ccc2c1NC(NCc1ccc3c(cnn3C)c1)=NS2(=O)=O] | (R)-6-fluoro-5-(1-(2- fluorophenyl)ethyl)-3-(((1-methyl- 1H-indazol-5-yl)methyl)amino)- 4H-benzo[e][1,2,4]thiadiazine 1,1- dioxide |
| 7-53 | 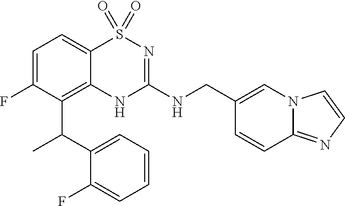 [SMILES: CC(c1ccccc1F)c1c(F)ccc2c1NC(NCc1ccc3nccn3c1)=NS2(=O)=O] | 6-fluoro-5-(1-(2- fluorophenyl)ethyl)-3- ((imidazo[1,2-a]pyridin-6- ylmethyl)amino)-4H- benzo[e][1,2,4]thiadiazine 1,1- dioxide |
| 7-53 (S) | 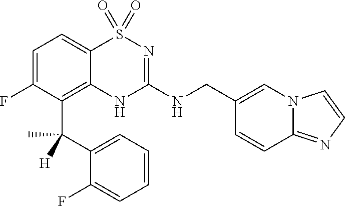 [SMILES: C[C@@H](c1ccccc1F)c1c(F)ccc2c1NC(NCc1ccc3nccn3c1)=NS2(=O)=O] | (S)-6-fluoro-5-(1-(2- fluorophenyl)ethyl)-3- ((imidazo[1,2-a]pyridin-6- ylmethyl)amino)-4H- benzo[e][1,2,4]thiadiazine 1,1- dioxide |
| 7-53 (R) | 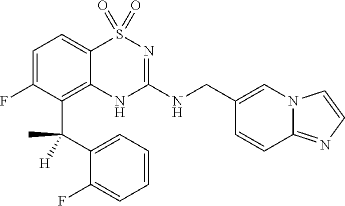 [SMILES: C[C@H](c1ccccc1F)c1c(F)ccc2c1NC(NCc1ccc3nccn3c1)=NS2(=O)=O] | (R)-6-fluoro-5-(1-(2- fluorophenyl)ethyl)-3- ((imidazo[1,2-a]pyridin-6- ylmethyl)amino)-4H- benzo[e][1,2,4]thiadiazine 1,1- dioxide |
| 7-54 | 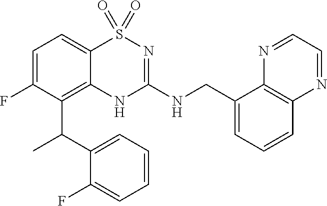 [SMILES: CC(c1ccccc1F)c1c(F)ccc2c1NC(NCc1cccc3nccnc13)=NS2(=O)=O] | 6-fluoro-5-(1-(2- fluorophenyl)ethyl)-3- ((quinoxalin-5-ylmethyl)amino)- 4H-benzo[e][1,2,4]thiadiazine 1,1- dioxide |
| 7-54 (S) | 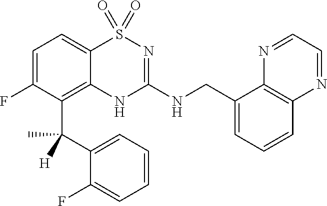 [SMILES: C[C@@H](c1ccccc1F)c1c(F)ccc2c1NC(NCc1cccc3nccnc13)=NS2(=O)=O] | (S)-6-fluoro-5-(1-(2- fluorophenyl)ethyl)-3- ((quinoxalin-5-ylmethyl)amino)- 4H-benzo[e][1,2,4]thiadiazine 1,1- dioxide |
| 7-54 (R) | 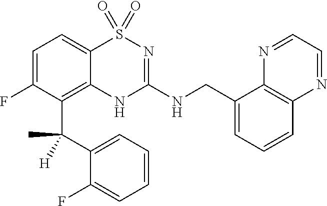 [SMILES: C[C@H](c1ccccc1F)c1c(F)ccc2c1NC(NCc1cccc3nccnc13)=NS2(=O)=O] | (R)-6-fluoro-5-(1-(2- fluorophenyl)ethyl)-3- ((quinoxalin-5-ylmethyl)amino)- 4H-benzo[e][1,2,4]thiadiazine 1,1- dioxide |
| 7-55 | 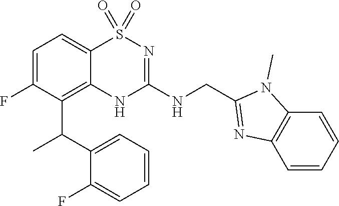 [SMILES: CC(c1ccccc1F)c1c(F)ccc2c1NC(NCc1nc3ccccc3n1C)=NS2(=O)=O] | 6-fluoro-5-(1-(2- fluorophenyl)ethyl)-3-(((1-methyl- 1H-benzo[d]imidazol-2- yl)methyl)amino)-4H- benzo[e][1,2,4]thiadiazine 1,1- dioxide |
| 7-55 (S) | 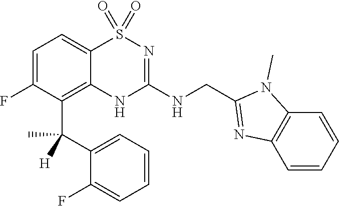 [SMILES: C[C@@H](c1ccccc1F)c1c(F)ccc2c1NC(NCc1nc3ccccc3n1C)=NS2(=O)=O] | (S)-6-fluoro-5-(1-(2- fluorophenyl)ethyl)-3-(((1-methyl- 1H-benzo[d]imidazol-2- yl)methyl)amino)-4H- benzo[e][1,2,4]thiadiazine 1,1- dioxide |
| 7-55 (R) | 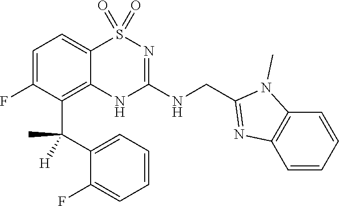 [SMILES: C[C@H](c1ccccc1F)c1c(F)ccc2c1NC(NCc1nc3ccccc3n1C)=NS2(=O)=O] | (R)-6-fluoro-5-(1-(2- fluorophenyl)ethyl)-3-(((1-methyl- 1H-benzo[d]imidazol-2- yl)methyl)amino)-4H- benzo[e][1,2,4]thiadiazine 1,1- dioxide |
| 7-56 | 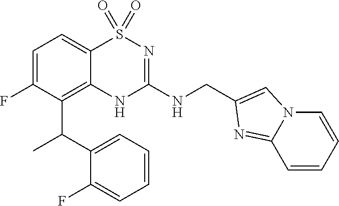 [SMILES: CC(c1ccccc1F)c1c(F)ccc2c1NC(NCc1cn3ccccc3n1)=NS2(=O)=O] | 6-fluoro-5-(1-(2- fluorophenyl)ethyl)-3- ((imidazo[1,2-a]pyridin-2- ylmethyl)amino)-4H- benzo[e][1,2,4]thiadiazine 1,1- dioxide |
| 7-56 (S) | 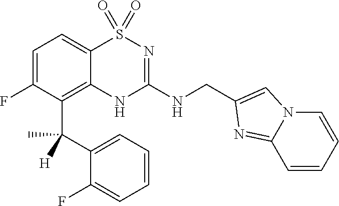 [SMILES: C[C@@H](c1ccccc1F)c1c(F)ccc2c1NC(NCc1cn3ccccc3n1)=NS2(=O)=O] | (S)-6-fluoro-5-(1-(2- fluorophenyl)ethyl)-3- ((imidazo[1,2-a]pyridin-2- ylmethyl)amino)-4H- benzo[e][1,2,4]thiadiazine 1,1- dioxide |
| 7-56 (R) | 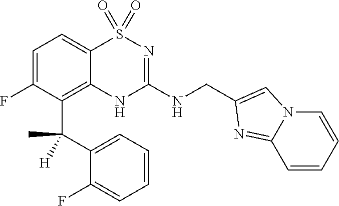 [SMILES: C[C@H](c1ccccc1F)c1c(F)ccc2c1NC(NCc1cn3ccccc3n1)=NS2(=O)=O] | (R)-6-fluoro-5-(1-(2- fluorophenyl)ethyl)-3- ((imidazo[1,2-a]pyridin-2- ylmethyl)amino)-4H- benzo[e][1,2,4]thiadiazine 1,1- dioxide |
| 7-57 | 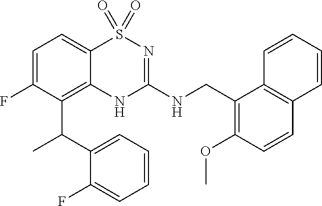 [SMILES: COc1ccc2ccccc2c1CNC1=NS(=O)(=O)c2ccc(F)c(C(C)c3ccccc3F)c2N1] | 6-fluoro-5-(1-(2- fluorophenyl)ethyl)-3-(((2- methoxynaphthalen-1- yl)methyl)amino)-4H- benzo[e][1,2,4]thiadiazine 1,1- dioxide |
| 7-57 (S) | 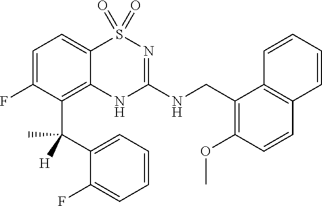 [SMILES: COc1ccc2ccccc2c1CNC1=NS(=O)(=O)c2ccc(F)c([C@@H](C)c3ccccc3F)c2N1] | (S)-6-fluoro-5-(1-(2- fluorophenyl)ethyl)-3-(((2- methoxynaphthalen-1- yl)methyl)amino)-4H- benzo[e][1,2,4]thiadiazine 1,1- dioxide |
| 7-57 (R) | 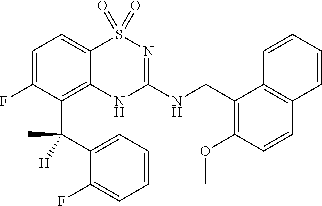 [SMILES: COc1ccc2ccccc2c1CNC1=NS(=O)(=O)c2ccc(F)c([C@H](C)c3ccccc3F)c2N1] | (R)-6-fluoro-5-(1-(2- fluorophenyl)ethyl)-3-(((2- methoxynaphthalen-1- yl)methyl)amino)-4H- benzo[e][1,2,4]thiadiazine 1,1- dioxide |
| 7-58 | 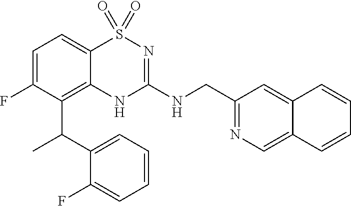 [SMILES: CC(c1ccccc1F)c1c(F)ccc2c1NC(NCc1cc3ccccc3cn1)=NS2(=O)=O] | 6-fluoro-5-(1-(2- fluorophenyl)ethyl)-3- ((isoquinolin-3-ylmethyl)amino)- 4H-benzo[e][1,2,4]thiadiazine 1,1- dioxide |
| 7-58 (S) | 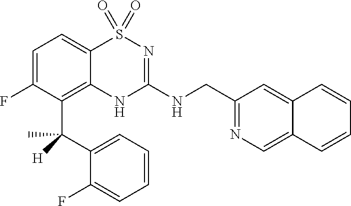 [SMILES: C[C@@H](c1ccccc1F)c1c(F)ccc2c1NC(NCc1cc3ccccc3cn1)=NS2(=O)=O] | (S)-6-fluoro-5-(1-(2- fluorophenyl)ethyl)-3- ((isoquinolin-3-ylmethyl)amino)- 4H-benzo[e][1,2,4]thiadiazine 1,1- dioxide |
| 7-58 (R) | 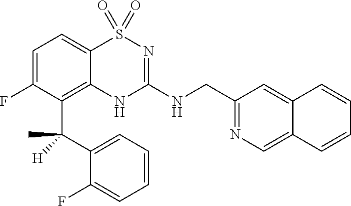 [SMILES: C[C@H](c1ccccc1F)c1c(F)ccc2c1NC(NCc1cc3ccccc3cn1)=NS2(=O)=O] | (R)-6-fluoro-5-(1-(2- fluorophenyl)ethyl)-3- ((isoquinolin-3-ylmethyl)amino)- 4H-benzo[e][1,2,4]thiadiazine 1,1- dioxide |
| 8-1 | 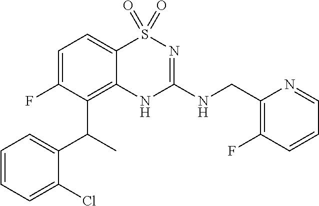 [SMILES: CC(c1ccccc1Cl)c1c(F)ccc2c1NC(NCc1ncccc1F)=NS2(=O)=O] | 5-(1-(2-chlorophenyl)ethyl)-6- fluoro-3-(((3-fluoropyridin-2- yl)methyl)amino)-4H- benzo[e][1,2,4]thiadiazine 1,1- dioxide |
| 8-1 (S) | 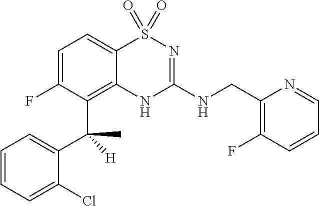 [SMILES: C[C@@H](c1ccccc1Cl)c1c(F)ccc2c1NC(NCc1ncccc1F)=NS2(=O)=O] | (S)-5-(1-(2-chlorophenyl)ethyl)-6- fluoro-3-(((3-fluoropyridin-2- yl)methyl)amino)-4H- benzo[e][1,2,4]thiadiazine 1,1- dioxide |
| 8-1 (R) | 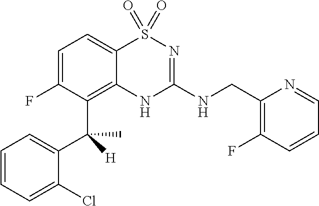 [SMILES: C[C@H](c1ccccc1Cl)c1c(F)ccc2c1NC(NCc1ncccc1F)=NS2(=O)=O] | (R)-5-(1-(2-chlorophenyl)ethyl)-6- fluoro-3-(((3-fluoropyridin-2- yl)methyl)amino)-4H- benzo[e][1,2,4]thiadiazine 1,1- dioxide |
| 8-2 | 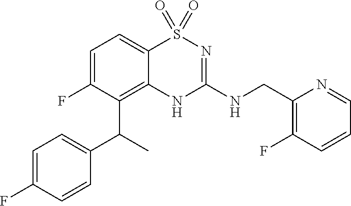 [SMILES: CC(c1ccc(F)cc1)c1c(F)ccc2c1NC(NCc1ncccc1F)=NS2(=O)=O] | 6-fluoro-5-(1-(4- fluorophenyl)ethyl)-3-(((3- fluoropyridin-2-yl)methyl)amino)- 4H-benzo[e][1,2,4]thiadiazine 1,1- dioxide |
| 8-2 (S) | 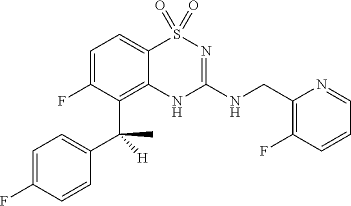 [SMILES: C[C@@H](c1ccc(F)cc1)c1c(F)ccc2c1NC(NCc1ncccc1F)=NS2(=O)=O] | (S)-6-fluoro-5-(1-(4- fluorophenyl)ethyl)-3-(((3- fluoropyridin-2-yl)methyl)amino)- 4H-benzo[e][1,2,4]thiadiazine 1,1- dioxide |
| 8-2 (R) | 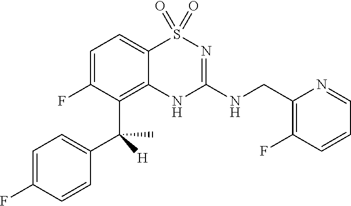 [SMILES: C[C@H](c1ccc(F)cc1)c1c(F)ccc2c1NC(NCc1ncccc1F)=NS2(=O)=O] | (R)-6-fluoro-5-(1-(4- fluorophenyl)ethyl)-3-(((3- fluoropyridin-2-yl)methyl)amino)- 4H-benzo[e][1,2,4]thiadiazine 1,1- dioxide |
| 8-3 | 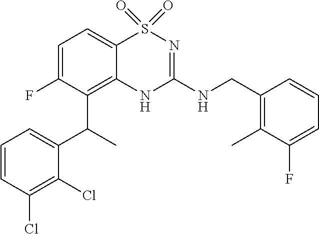 [SMILES: Cc1c(F)cccc1CNC1=NS(=O)(=O)c2ccc(F)c(C(C)c3cccc(Cl)c3Cl)c2N1] | 5-(1-(2,3-dichlorophenyl)ethyl)-6- fluoro-3-((3-fluoro-2- methylbenzyl)amino)-4H- benzo[e][1,2,4]thiadiazine 1,1- dioxide |
| 8-3 (S) | 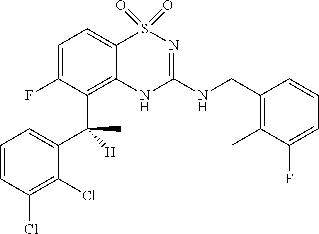 [SMILES: Cc1c(F)cccc1CNC1=NS(=O)(=O)c2ccc(F)c([C@@H](C)c3cccc(Cl)c3Cl)c2N1] | (S)-5-(1-(2,3- dichlorophenyl)ethyl)-6-fluoro-3- ((3-fluoro-2-methylbenzyl)amino)- 4H-benzo[e][1,2,4]thiadiazine 1,1- dioxide |
| 8-3 (R) | 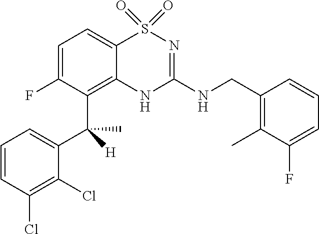 [SMILES: Cc1c(F)cccc1CNC1=NS(=O)(=O)c2ccc(F)c([C@H](C)c3cccc(Cl)c3Cl)c2N1] | (R)-5-(1-(2,3- dichlorophenyl)ethyl)-6-fluoro-3- ((3-fluoro-2-methylbenzyl)amino)- 4H-benzo[e][1,2,4]thiadiazine 1,1- dioxide |
| 9-1 | 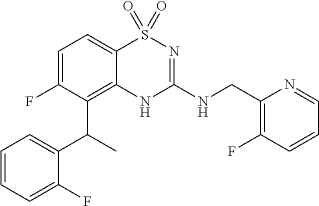 [SMILES: CC(c1ccccc1F)c1c(F)ccc2c1NC(NCc1ncccc1F)=NS2(=O)=O] | 6-fluoro-5-(1-(2- fluorophenyl)ethyl)-3-(((3- fluoropyridin-2-yl)methyl)amino)- 4H-benzo[e][1,2,4]thiadiazine 1,1- dioxide |
| 9-1 (S) | 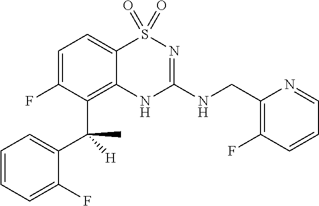 [SMILES: C[C@@H](c1ccccc1F)c1c(F)ccc2c1NC(NCc1ncccc1F)=NS2(=O)=O] | (S)-6-fluoro-5-(1-(2- fluorophenyl)ethyl)-3-(((3- fluoropyridin-2-yl)methyl)amino)- 4H-benzo[e][1,2,4]thiadiazine 1,1- dioxide |
| 9-1 (R) | 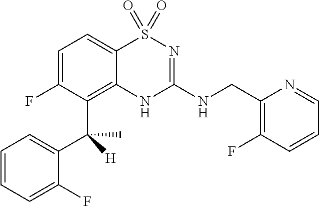 [SMILES: C[C@H](c1ccccc1F)c1c(F)ccc2c1NC(NCc1ncccc1F)=NS2(=O)=O] | (R)-6-fluoro-5-(1-(2- fluorophenyl)ethyl)-3-(((3- fluoropyridin-2-yl)methyl)amino)- 4H-benzo[e][1,2,4]thiadiazine 1,1- dioxide |
| 9-2 | 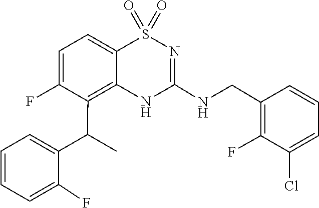 [SMILES: CC(c1ccccc1F)c1c(F)ccc2c1NC(NCc1cccc(Cl)c1F)=NS2(=O)=O] | 3-((3-chloro-2- fluorobenzyl)amino)-6-fluoro-5-(1- (2-fluorophenyl)ethyl)-4H- benzo[e][1,2,4]thiadiazine 1,1- dioxide |
| 9-2 (S) | 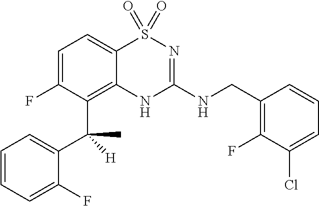 [SMILES: C[C@@H](c1ccccc1F)c1c(F)ccc2c1NC(NCc1cccc(Cl)c1F)=NS2(=O)=O] | (S)-3-((3-chloro-2- fluorobenzyl)amino)-6-fluoro-5-(1- (2-fluorophenyl)ethyl)-4H- benzo[e][1,2,4]thiadiazine 1,1- dioxide |
| 9-2 (R) | 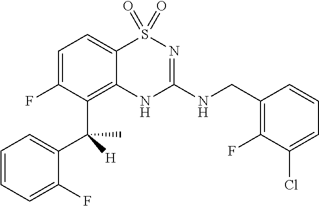 [SMILES: C[C@H](c1ccccc1F)c1c(F)ccc2c1NC(NCc1cccc(Cl)c1F)=NS2(=O)=O] | (R)-3-((3-chloro-2- fluorobenzyl)amino)-6-fluoro-5-(1- (2-fluorophenyl)ethyl)-4H- benzo[e][1,2,4]thiadiazine 1,1- dioxide |
| 9-3 | 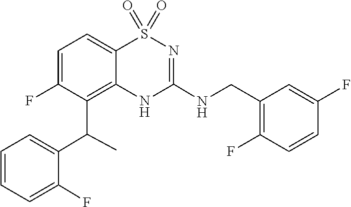 [SMILES: CC(c1ccccc1F)c1c(F)ccc2c1NC(NCc1cc(F)ccc1F)=NS2(=O)=O] | 3-((2,5-difluorobenzyl)amino)-6- fluoro-5-(1-(2-fluorophenyl)ethyl)- 4H-benzo[e][1,2,4]thiadiazine 1,1- dioxide |
| 9-3 (S) | 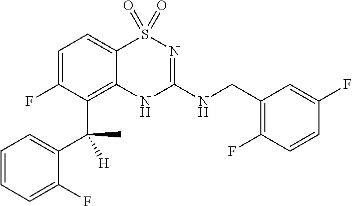 [SMILES: C[C@@H](c1ccccc1F)c1c(F)ccc2c1NC(NCc1cc(F)ccc1F)=NS2(=O)=O] | (S)-3-((2,5-difluorobenzyl)amino)- 6-fluoro-5-(1-(2- fluorophenyl)ethyl)-4H- benzo[e][1,2,4]thiadiazine 1,1- dioxide |
| 9-3 (R) | 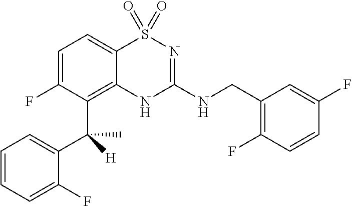 [SMILES: C[C@H](c1ccccc1F)c1c(F)ccc2c1NC(NCc1cc(F)ccc1F)=NS2(=O)=O] | (R)-3-((2,5-difluorobenzyl)amino)- 6-fluoro-5-(1-(2- fluorophenyl)ethyl)-4H- benzo[e][1,2,4]thiadiazine 1,1- dioxide |
| 9-4 | 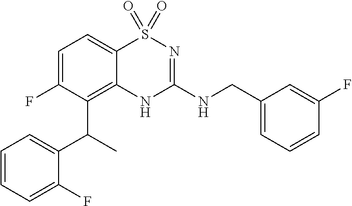 [SMILES: CC(c1ccccc1F)c1c(F)ccc2c1NC(NCc1cccc(F)c1)=NS2(=O)=O] | 6-fluoro-3-((3- fluorobenzyl)amino)-5-(1-(2- fluorophenyl)ethyl)-4H- benzo[e][1,2,4]thiadiazine 1,1- dioxide |
| 9-4 (S) | 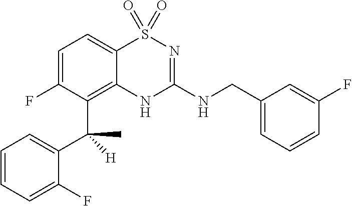 [SMILES: C[C@@H](c1ccccc1F)c1c(F)ccc2c1NC(NCc1cccc(F)c1)=NS2(=O)=O] | (S)-6-fluoro-3-((3- fluorobenzyl)amino)-5-(1-(2- fluorophenyl)ethyl)-4H- benzo[e][1,2,4]thiadiazine 1,1- dioxide |
| 9-4 (R) | 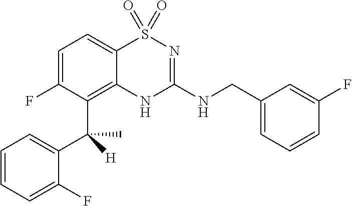 [SMILES: C[C@H](c1ccccc1F)c1c(F)ccc2c1NC(NCc1cccc(F)c1)=NS2(=O)=O] | (R)-6-fluoro-3-((3- fluorobenzyl)amino)-5-(1-(2- fluorophenyl)ethyl)-4H- benzo[e][1,2,4]thiadiazine 1,1- dioxide |
| 9-5 | 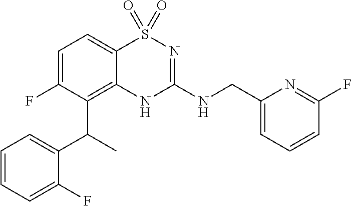 [SMILES: CC(c1ccccc1F)c1c(F)ccc2c1NC(NCc1cccc(F)n1)=NS2(=O)=O] | 6-fluoro-5-(1-(2- fluorophenyl)ethyl)-3-(((6- fluoropyridin-2-yl)methyl)amino)- 4H-benzo[e][1,2,4]thiadiazine 1,1- dioxide |
| 9-5 (S) | 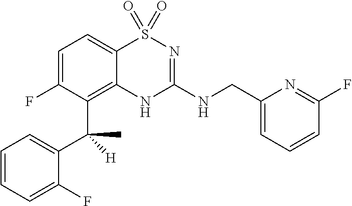 [SMILES: C[C@@H](c1ccccc1F)c1c(F)ccc2c1NC(NCc1cccc(F)n1)=NS2(=O)=O] | (S)-6-fluoro-5-(1-(2- fluorophenyl)ethyl)-3-(((6- fluoropyridin-2-yl)methyl)amino)- 4H-benzo[e][1,2,4]thiadiazine 1,1- dioxide |
| 9-5 (R) | 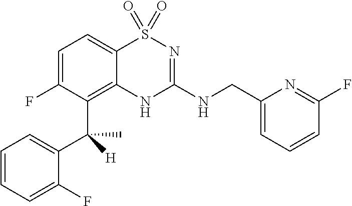 [SMILES: C[C@H](c1ccccc1F)c1c(F)ccc2c1NC(NCc1cccc(F)n1)=NS2(=O)=O] | (R)-6-fluoro-5-(1-(2- fluorophenyl)ethyl)-3-(((6- fluoropyridin-2-yl)methyl)amino)- 4H-benzo[e][1,2,4]thiadiazine 1,1- dioxide |
| 9-6 | 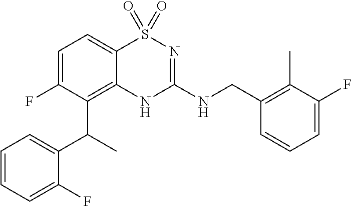 [SMILES: Cc1c(F)cccc1CNC1=NS(=O)(=O)c2ccc(F)c(C(C)c3ccccc3F)c2N1] | 6-fluoro-3-((3-fluoro-2- methylbenzyl)amino)-5-(1-(2- fluorophenyl)ethyl)-4H- benzo[e][1,2,4]thiadiazine 1,1- dioxide |
| 9-6 (S) | 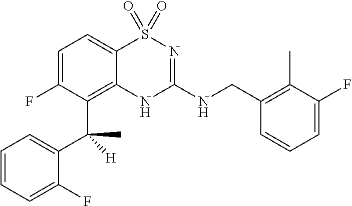 [SMILES: Cc1c(F)cccc1CNC1=NS(=O)(=O)c2ccc(F)c([C@@H](C)c3ccccc3F)c2N1] | (S)-6-fluoro-3-((3-fluoro-2- methylbenzyl)amino)-5-(1-(2- fluorophenyl)ethyl)-4H- benzo[e][1,2,4]thiadiazine 1,1- dioxide |
| 9-6 (R) | 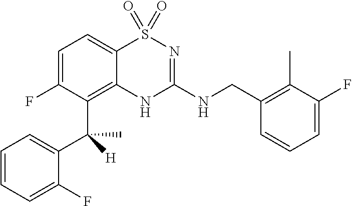 [SMILES: Cc1c(F)cccc1CNC1=NS(=O)(=O)c2ccc(F)c([C@H](C)c3ccccc3F)c2N1] | (R)-6-fluoro-3-((3-fluoro-2- methylbenzyl)amino)-5-(1-(2- fluorophenyl)ethyl)-4H- benzo[e][1,2,4]thiadiazine 1,1- dioxide |
| 9-7 | 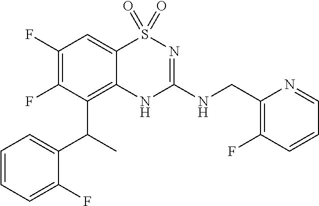 [SMILES: CC(c1ccccc1F)c1c(F)c(F)cc2c1NC(NCc1ncccc1F)=NS2(=O)=O] | 6,7-difluoro-5-(1-(2- fluorophenyl)ethyl)-3-(((3- fluoropyridin-2-yl)methyl)amino)- 4H-benzo[e][1,2,4]thiadiazine 1,1- dioxide |
| 9-7 (S) | 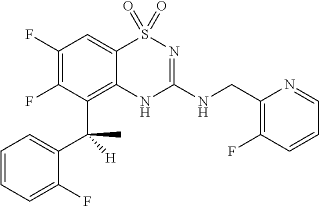 [SMILES: C[C@@H](c1ccccc1F)c1c(F)c(F)cc2c1NC(NCc1ncccc1F)=NS2(=O)=O] | (S)-6,7-difluoro-5-(1-(2- fluorophenyl)ethyl)-3-(((3- fluoropyridin-2-yl)methyl)amino)- 4H-benzo[e][1,2,4]thiadiazine 1,1- dioxide |
| 9-7 (R) | 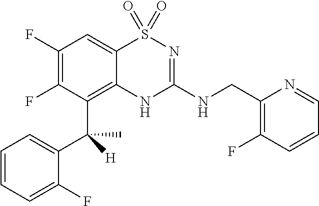 [SMILES: C[C@H](c1ccccc1F)c1c(F)c(F)cc2c1NC(NCc1ncccc1F)=NS2(=O)=O] | (R)-6,7-difluoro-5-(1-(2- fluorophenyl)ethyl)-3-(((3- fluoropyridin-2-yl)methyl)amino)- 4H-benzo[e][1,2,4]thiadiazine 1,1- dioxide |
| 10-1 | 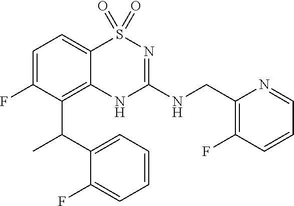 [SMILES: CC(c1ccccc1F)c1c(F)ccc2c1NC(NCc1ncccc1F)=NS2(=O)=O] | 6-fluoro-5-(1-(2- fluorophenyl)ethyl)-3-(((3- fluoropyridin-2-yl)methyl)amino)- 4H-benzo[e][1,2,4]thiadiazine 1,1- dioxide |
| 10-1A | 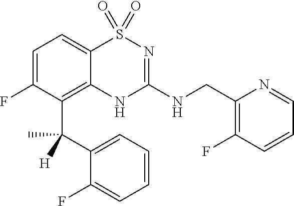 [SMILES: C[C@@H](c1ccccc1F)c1c(F)ccc2c1NC(NCc1ncccc1F)=NS2(=O)=O] | (S)-6-fluoro-5-(1-(2- fluorophenyl)ethyl)-3-(((3- fluoropyridin-2-yl)methyl)amino)- 4H-benzo[e][1,2,4]thiadiazine 1,1- dioxide |
| 10-1B | 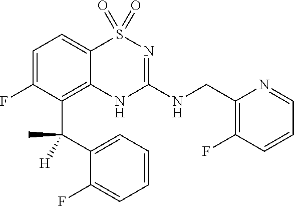 [SMILES: C[C@H](c1ccccc1F)c1c(F)ccc2c1NC(NCc1ncccc1F)=NS2(=O)=O] | (R)-6-fluoro-5-(1-(2- fluorophenyl)ethyl)-3-(((3- fluoropyridin-2-yl)methyl)amino)- 4H-benzo[e][1,2,4]thiadiazine 1,1- dioxide |
| 10-2 | 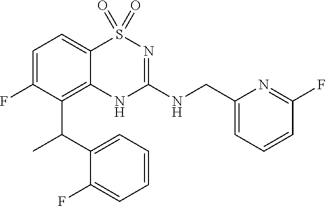 [SMILES: CC(c1ccccc1F)c1c(F)ccc2c1NC(NCc1cccc(F)n1)=NS2(=O)=O] | 6-fluoro-5-(1-(2- fluorophenyl)ethyl)-3-(((6- fluoropyridin-2-yl)methyl)amino)- 4H-benzo[e][1,2,4]thiadiazine 1,1- dioxide |
| 10-2A | 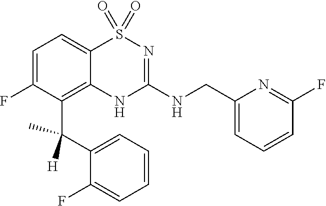 [SMILES: C[C@@H](c1ccccc1F)c1c(F)ccc2c1NC(NCc1cccc(F)n1)=NS2(=O)=O] | (S)-6-fluoro-5-(1-(2- fluorophenyl)ethyl)-3-(((6- fluoropyridin-2-yl)methyl)amino)- 4H-benzo[e][1,2,4]thiadiazine 1,1- dioxide |
| 10-2B | 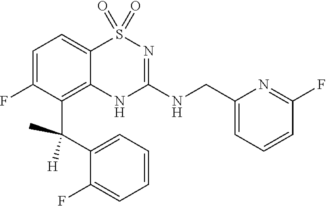 [SMILES: C[C@H](c1ccccc1F)c1c(F)ccc2c1NC(NCc1cccc(F)n1)=NS2(=O)=O] | (R)-6-fluoro-5-(1-(2- fluorophenyl)ethyl)-3-(((6- fluoropyridin-2-yl)methyl)amino)- 4H-benzo[e][1,2,4]thiadiazine 1,1- dioxide |
| 10-3 | 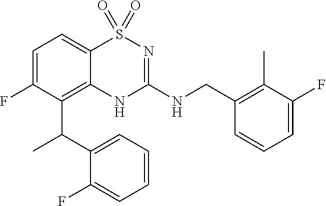 [SMILES: Cc1c(F)cccc1CNC1=NS(=O)(=O)c2ccc(F)c(C(C)c3ccccc3F)c2N1] | 6-fluoro-3-((3-fluoro-2- methylbenzyl)amino)-5-(1-(2- fluorophenyl)ethyl)-4H- benzo[e][1,2,4]thiadiazine 1,1- dioxide |
| 10-3A | 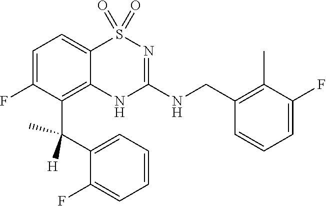 [SMILES: Cc1c(F)cccc1CNC1=NS(=O)(=O)c2ccc(F)c([C@@H](C)c3ccccc3F)c2N1] | (S)-6-fluoro-3-((3-fluoro-2- methylbenzyl)amino)-5-(1-(2- fluorophenyl)ethyl)-4H- benzo[e][1,2,4]thiadiazine 1,1- dioxide |
| 10-3B | 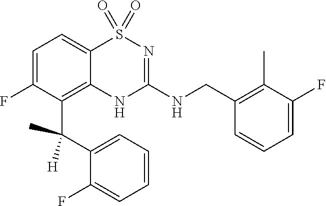 [SMILES: Cc1c(F)cccc1CNC1=NS(=O)(=O)c2ccc(F)c([C@H](C)c3ccccc3F)c2N1] | (R)-6-fluoro-3-((3-fluoro-2- methylbenzyl)amino)-5-(1-(2- fluorophenyl)ethyl)-4H- benzo[e][1,2,4]thiadiazine 1,1- dioxide |
| 10-4 | 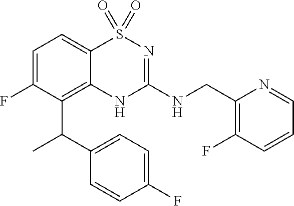 [SMILES: CC(c1ccc(F)cc1)c1c(F)ccc2c1NC(NCc1ncccc1F)=NS2(=O)=O] | 6-fluoro-5-(1-(4- fluorophenyl)ethyl)-3-(((3- fluoropyridin-2-yl)methyl)amino)- 4H-benzo[e][1,2,4]thiadiazine 1,1- dioxide |
| 10-4A | 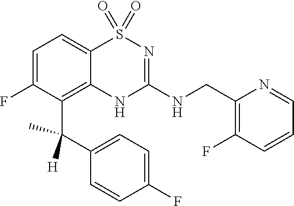 [SMILES: C[C@@H](c1ccc(F)cc1)c1c(F)ccc2c1NC(NCc1ncccc1F)=NS2(=O)=O] | (S)-6-fluoro-5-(1-(4- fluorophenyl)ethyl)-3-(((3- fluoropyridin-2-yl)methyl)amino)- 4H-benzo[e][1,2,4]thiadiazine 1,1- dioxide |
| 10-4B | 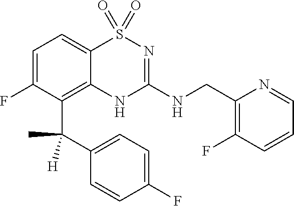 [SMILES: C[C@H](c1ccc(F)cc1)c1c(F)ccc2c1NC(NCc1ncccc1F)=NS2(=O)=O] | (R)-6-fluoro-5-(1-(4- fluorophenyl)ethyl)-3-(((3- fluoropyridin-2-yl)methyl)amino)- 4H-benzo[e][1,2,4]thiadiazine 1,1- dioxide |
| 10-5 | 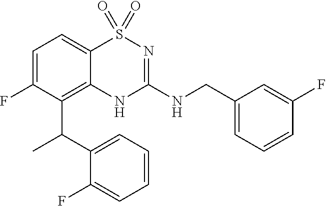 [SMILES: CC(c1ccccc1F)c1c(F)ccc2c1NC(NCc1cccc(F)c1)=NS2(=O)=O] | 6-fluoro-3-((3- fluorobenzyl)amino)-5-(1-(2- fluorophenyl)ethyl)-4H- benzo[e][1,2,4]thiadiazine 1,1- dioxide |
| 10-5A | 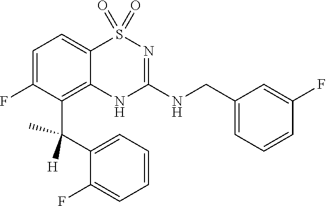 [SMILES: C[C@@H](c1ccccc1F)c1c(F)ccc2c1NC(NCc1cccc(F)c1)=NS2(=O)=O] | (S)-6-fluoro-3-((3- fluorobenzyl)amino)-5-(1-(2- fluorophenyl)ethyl)-4H- benzo[e][1,2,4]thiadiazine 1,1- dioxide |
| 10-5B | 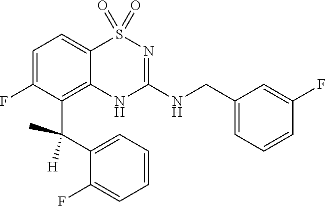 [SMILES: C[C@H](c1ccccc1F)c1c(F)ccc2c1NC(NCc1cccc(F)c1)=NS2(=O)=O] | (R)-6-fluoro-3-((3- fluorobenzyl)amino)-5-(1-(2- fluorophenyl)ethyl)-4H- benzo[e][1,2,4]thiadiazine 1,1- dioxide |
| 10-6 | 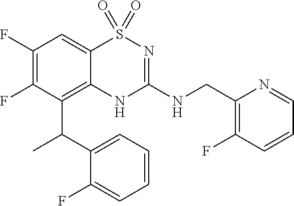 [SMILES: CC(c1ccccc1F)c1c(F)c(F)cc2c1NC(NCc1ncccc1F)=NS2(=O)=O] | 6,7-difluoro-5-(1-(2- fluorophenyl)ethyl)-3-(((3- fluoropyridin-2-yl)methyl)amino)- 4H-benzo[e][1,2,4]thiadiazine 1,1- dioxide |
| 10-6A | 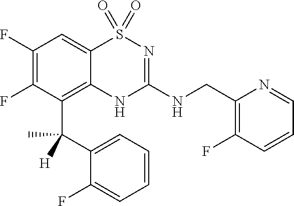 [SMILES: C[C@@H](c1ccccc1F)c1c(F)c(F)cc2c1NC(NCc1ncccc1F)=NS2(=O)=O] | (S)-6,7-difluoro-5-(1-(2- fluorophenyl)ethyl)-3-(((3- fluoropyridin-2-yl)methyl)amino)- 4H-benzo[e][1,2,4]thiadiazine 1,1- dioxide |
| 10-6B | 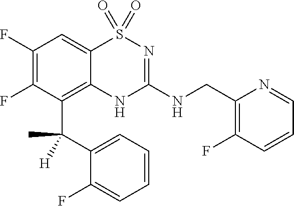 [SMILES: C[C@H](c1ccccc1F)c1c(F)c(F)cc2c1NC(NCc1ncccc1F)=NS2(=O)=O] | (R)-6,7-difluoro-5-(1-(2- fluorophenyl)ethyl)-3-(((3- fluoropyridin-2-yl)methyl)amino)- 4H-benzo[e][1,2,4]thiadiazine 1,1- dioxide |
| 11-1 | 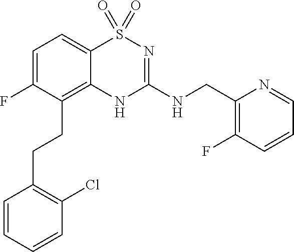 [SMILES: O=S1(=O)N=C(NCc2ncccc2F)Nc2c1ccc(F)c2CCc1ccccc1Cl] | 5-(2-chlorophenethyl)-6-fluoro-3- (((3-fluoropyridin-2- yl)methyl)amino)-4H- benzo[e][1,2,4]thiadiazine 1,1- dioxide |
| 12-1 | 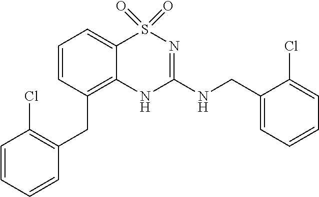 [SMILES: O=S1(=O)N=C(NCc2ccccc2Cl)Nc2c(Cc3ccccc3Cl)cccc21] | 5-(2-chlorobenzyl)-3-((2- chlorobenzyl)amino)-4H- benzo[e][1,2,4]thiadiazine 1,1- dioxide |
| 12-2 | 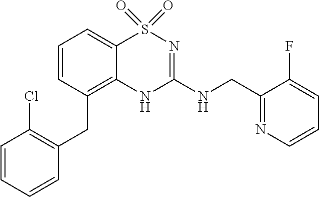 [SMILES: O=S1(=O)N=C(NCc2ncccc2F)Nc2c(Cc3ccccc3Cl)cccc21] | 5-(2-chlorobenzyl)-3-(((3- fluoropyridin-2-yl)methyl)amino)- 4H-benzo[e][1,2,4]thiadiazine 1,1- dioxide |
| 12-3 | 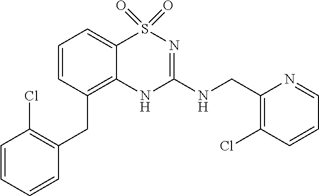 [SMILES: O=S1(=O)N=C(NCc2ncccc2Cl)Nc2c(Cc3ccccc3Cl)cccc21] | 5-(2-chlorobenzyl)-3-(((3- chloropyridin-2-yl)methyl)amino)- 4H-benzo[e][1,2,4]thiadiazine 1,1- dioxide |
| 12-4 | 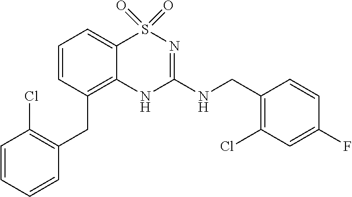 [SMILES: O=S1(=O)N=C(NCc2ccc(F)cc2Cl)Nc2c(Cc3ccccc3Cl)cccc21] | 3-((2-chloro-4- fluorobenzyl)amino)-5-(2- chlorobenzyl)-4H- benzo[e][1,2,4]thiadiazine 1,1- dioxide |
| 12-5 | 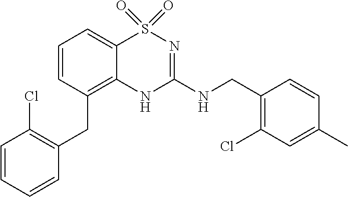 [SMILES: Cc1ccc(CNC2=NS(=O)(=O)c3cccc(Cc4ccccc4Cl)c3N2)c(Cl)c1] | 3-((2-chloro-4- methylbenzyl)amino)-5-(2- chlorobenzyl)-4H- benzo[e][1,2,4]thiadiazine 1,1- dioxide |
| 12-6 | 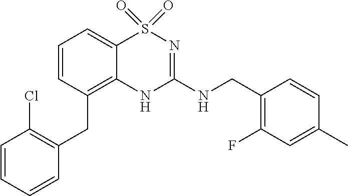 [SMILES: Cc1ccc(CNC2=NS(=O)(=O)c3cccc(Cc4ccccc4Cl)c3N2)c(F)c1] | 5-(2-chlorobenzyl)-3-((2-fluoro-4- methylbenzyl)amino)-4H- benzo[e][1,2,4]thiadiazine 1,1- dioxide |
| 12-7 | 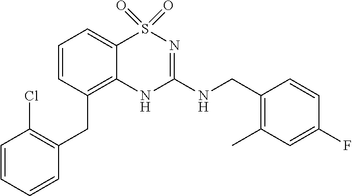 [SMILES: Cc1cc(F)ccc1CNC1=NS(=O)(=O)c2cccc(Cc3ccccc3Cl)c2N1] | 5-(2-chlorobenzyl)-3-((4-fluoro-2- methylbenzyl)amino)-4H- benzo[e][1,2,4]thiadiazine 1,1- dioxide |
| 12-8 | 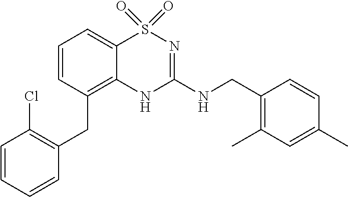 [SMILES: Cc1ccc(CNC2=NS(=O)(=O)c3cccc(Cc4ccccc4Cl)c3N2)c(C)c1] | 5-(2-chlorobenzyl)-3-((2,4- dimethylbenzyl)amino)-4H- benzo[e][1,2,4]thiadiazine 1,1- dioxide |
| 12-9 | 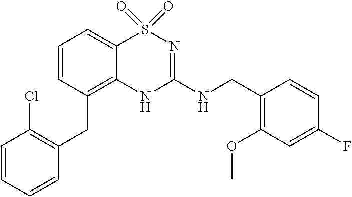 [SMILES: COc1cc(F)ccc1CNC1=NS(=O)(=O)c2cccc(Cc3ccccc3Cl)c2N1] | 5-(2-chlorobenzyl)-3-((4-fluoro-2- methoxybenzyl)amino)-4H- benzo[e][1,2,4]thiadiazine 1,1- dioxide |
| 12-10 | 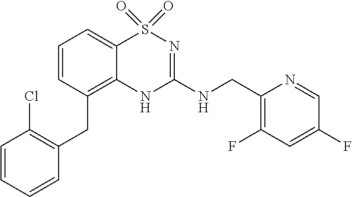 [SMILES: O=S1(=O)N=C(NCc2ncc(F)cc2F)Nc2c(Cc3ccccc3Cl)cccc21] | 5-(2-chlorobenzyl)-3-(((3,5- difluoropyridin-2- yl)methyl)amino)-4H- benzo[e][1,2,4]thiadiazine 1,1- dioxide |
| 12-11 | 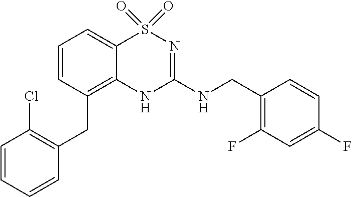 [SMILES: O=S1(=O)N=C(NCc2ccc(F)cc2F)Nc2c(Cc3ccccc3Cl)cccc21] | 5-(2-chlorobenzyl)-3-((2,4- difluorobenzyl)amino)-4H- benzo[e][1,2,4]thiadiazine 1,1- dioxide |
| 12-12 | 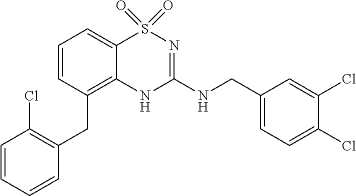 [SMILES: O=S1(=O)N=C(NCc2ccc(Cl)c(Cl)c2)Nc2c(Cc3ccccc3Cl)cccc21] | 5-(2-chlorobenzyl)-3-((3,4- dichlorobenzyl)amino)-4H- benzo[e][1,2,4]thiadiazine 1,1- dioxide |
| 12-13 | 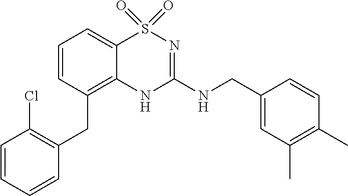 [SMILES: Cc1ccc(CNC2=NS(=O)(=O)c3cccc(Cc4ccccc4Cl)c3N2)cc1C] | 5-(2-chlorobenzyl)-3-((3,4- dimethylbenzyl)amino)-4H- benzo[e][1,2,4]thiadiazine 1,1- dioxide |
| 12-14 | 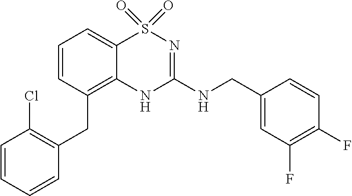 [SMILES: O=S1(=O)N=C(NCc2ccc(F)c(F)c2)Nc2c(Cc3ccccc3Cl)cccc21] | 5-(2-chlorobenzyl)-3-((3,4- difluorobenzyl)amino)-4H- benzo[e][1,2,4]thiadiazine 1,1- dioxide |
| 12-15 | 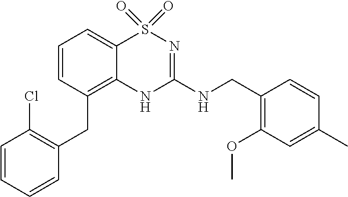 [SMILES: COc1cc(C)ccc1CNC1=NS(=O)(=O)c2cccc(Cc3ccccc3Cl)c2N1] | 5-(2-chlorobenzyl)-3-((2-methoxy- 4-methylbenzyl)amino)-4H- benzo[e][1,2,4]thiadiazine 1,1- dioxide |
| 12-16 | 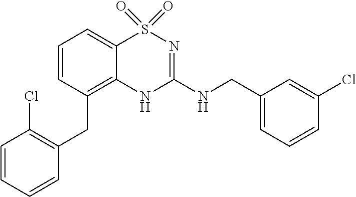 [SMILES: O=S1(=O)N=C(NCc2cccc(Cl)c2)Nc2c(Cc3ccccc3Cl)cccc21] | 5-(2-chlorobenzyl)-3-((3- chlorobenzyl)amino)-4H- benzo[e][1,2,4]thiadiazine 1,1- dioxide |
| 12-17 | 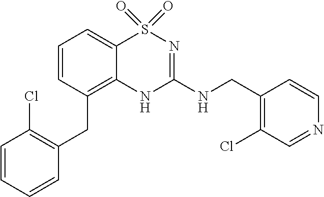 [SMILES: O=S1(=O)N=C(NCc2ccncc2Cl)Nc2c(Cc3ccccc3Cl)cccc21] | 5-(2-chlorobenzyl)-3-(((3- chloropyridin-4-yl)methyl)amino)- 4H-benzo[e][1,2,4]thiadiazine 1,1- dioxide |
| 12-18 | 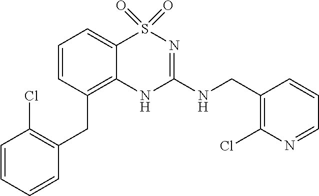 [SMILES: O=S1(=O)N=C(NCc2cccnc2Cl)Nc2c(Cc3ccccc3Cl)cccc21] | 5-(2-chlorobenzyl)-3-(((2- chloropyridin-3-yl)methyl)amino)- 4H-benzo[e][1,2,4]thiadiazine 1,1- dioxide |
| 12-19 | 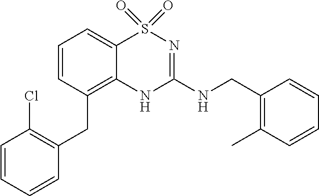 [SMILES: Cc1ccccc1CNC1=NS(=O)(=O)c2cccc(Cc3ccccc3Cl)c2N1] | 5-(2-chlorobenzyl)-3-((2- methylbenzyl)amino)-4H- benzo[e][1,2,4]thiadiazine 1,1- dioxide |
| 12-20 | 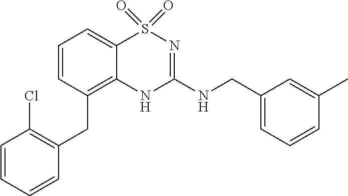 [SMILES: Cc1cccc(CNC2=NS(=O)(=O)c3cccc(Cc4ccccc4Cl)c3N2)c1] | 5-(2-chlorobenzyl)-3-((3- methylbenzyl)amino)-4H- benzo[e][1,2,4]thiadiazine 1,1- dioxide |
| 12-21 | 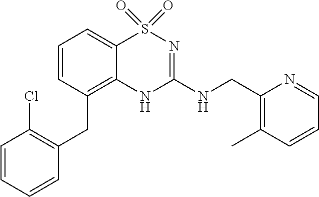 [SMILES: Cc1cccnc1CNC1=NS(=O)(=O)c2cccc(Cc3ccccc3Cl)c2N1] | 5-(2-chlorobenzyl)-3-(((3- methylpyridin-2-yl)methyl)amino)- 4H-benzo[e][1,2,4]thiadiazine 1,1- dioxide |
| 12-22 | 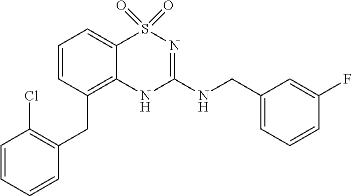 [SMILES: O=S1(=O)N=C(NCc2cccc(F)c2)Nc2c(Cc3ccccc3Cl)cccc21] | 5-(2-chlorobenzyl)-3-((3- fluorobenzyl)amino)-4H- benzo[e][1,2,4]thiadiazine 1,1- dioxide |
| 12-23 | 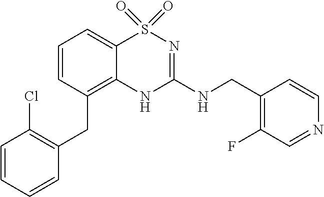 [SMILES: O=S1(=O)N=C(NCc2ccncc2F)Nc2c(Cc3ccccc3Cl)cccc21] | 5-(2-chlorobenzyl)-3-(((3- fluoropyridin-4-yl)methyl)amino)- 4H-benzo[e][1,2,4]thiadiazine 1,1- dioxide |
| 12-24 | 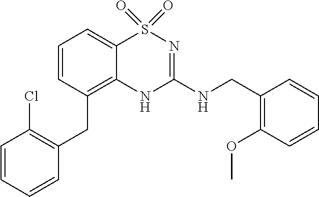 [SMILES: COc1ccccc1CNC1=NS(=O)(=O)c2cccc(Cc3ccccc3Cl)c2N1] | 5-(2-chlorobenzyl)-3-((2- methoxybenzyl)amino)-4H- benzo[e][1,2,4]thiadiazine 1,1- dioxide |
| 12-25 | 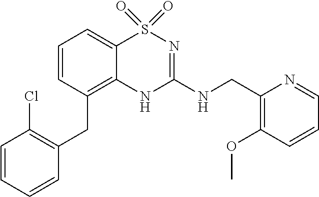 [SMILES: COc1cccnc1CNC1=NS(=O)(=O)c2cccc(Cc3ccccc3Cl)c2N1] | 5-(2-chlorobenzyl)-3-(((3- methoxypyridin-2- yl)methyl)amino)-4H- benzo[e][1,2,4]thiadiazine 1,1- dioxide |
| 12-26 | 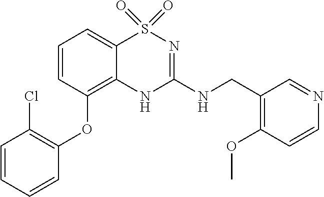 [SMILES: COc1ccncc1CNC1=NS(=O)(=O)c2cccc(Oc3ccccc3Cl)c2N1] | 5-(2-chlorobenzyl)-3-(((4- methoxypyridin-3- yl)methyl)amino)-4H- benzo[e][1,2,4]thiadiazine 1,1- dioxide |
| 12-27 | 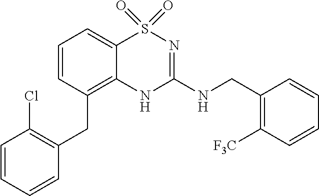 [SMILES: O=S1(=O)N=C(NCc2ccccc2C(F)(F)F)Nc2c(Cc3ccccc3Cl)cccc21] | 5-(2-chlorobenzyl)-3-((2- (trifluoromethyl)benzyl)amino)- 4H-benzo[e][1,2,4]thiadiazine 1,1- dioxide |
| 12-28 | 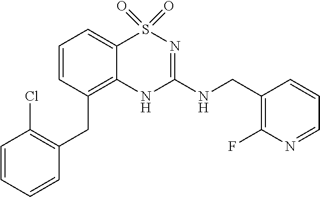 [SMILES: O=S1(=O)N=C(NCc2cccnc2F)Nc2c(Cc3ccccc3Cl)cccc21] | 5-(2-chlorobenzyl)-3-(((2- fluoropyridin-3-yl)methyl)amino)- 4H-benzo[e][1,2,4]thiadiazine 1,1- dioxide |
| 12-29 | 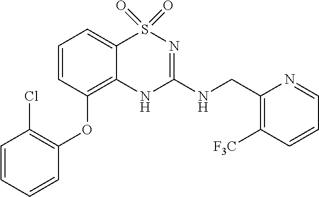 [SMILES: O=S1(=O)N=C(NCc2ncccc2C(F)(F)F)Nc2c(Oc3ccccc3Cl)cccc21] | 5-(2-chlorobenzyl)-3-(((3- (trifluoromethyl)pyridin-2- yl)methyl)amino)-4H- benzo[e][1,2,4]thiadiazine 1,1- dioxide |
| 12-30 | 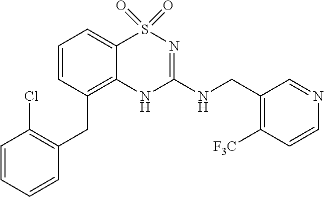 [SMILES: O=S1(=O)N=C(NCc2cnccc2C(F)(F)F)Nc2c(Cc3ccccc3Cl)cccc21] | 5-(2-chlorobenzyl)-3-(((4- (trifluoromethyl)pyridin-3- yl)methyl)amino)-4H- benzo[e][1,2,4]thiadiazine 1,1- dioxide |
| 12-31 | 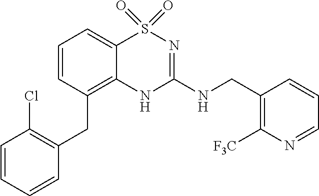 [SMILES: O=S1(=O)N=C(NCc2cccnc2C(F)(F)F)Nc2c(Cc3ccccc3Cl)cccc21] | 5-(2-chlorobenzyl)-3-(((2- (trifluoromethyl)pyridin-3- yl)methyl)amino)-4H- benzo[e][1,2,4]thiadiazine 1,1- dioxide |
| 12-32 | 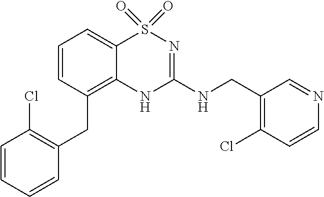 [SMILES: O=S1(=O)N=C(NCc2cnccc2Cl)Nc2c(Cc3ccccc3Cl)cccc21] | 5-(2-chlorobenzyl)-3-(((4- chloropyridin-3-yl)methyl)amino)- 4H-benzo[e][1,2,4]thiadiazine 1,1- dioxide |
| 13-1 | 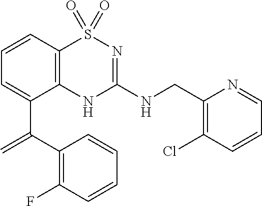 [SMILES: C=C(c1ccccc1F)c1cccc2c1NC(NCc1ncccc1Cl)=NS2(=O)=O] | 3-(((3-chloropyridin-2- yl)methyl)amino)-5-(1-(2- fluorophenyl)vinyl)-4H- benzo[e][1,2,4]thiadiazine 1,1- dioxide |
| 13-2 | 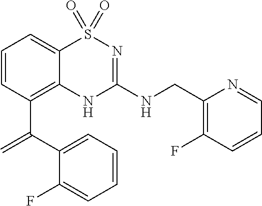 [SMILES: C=C(c1ccccc1F)c1cccc2c1NC(NCc1ncccc1F)=NS2(=O)=O] | 5-(1-(2-fluorophenyl)vinyl)-3-(((3- fluoropyridin-2-yl)methyl)amino)- 4H-benzo[e][1,2,4]thiadiazine 1,1- dioxide |
| 13-3 | 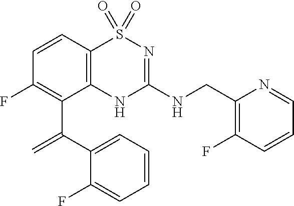 [SMILES: C=C(c1ccccc1F)c1c(F)ccc2c1NC(NCc1ncccc1F)=NS2(=O)=O] | 6-fluoro-5-(1-(2- fluorophenyl)vinyl)-3-(((3- fluoropyridin-2-yl)methyl)amino)- 4H-benzo[e][1,2,4]thiadiazine 1,1- dioxide |
| 14-1 (racemic) | 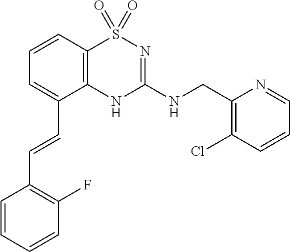 [SMILES: O=S1(=O)N=C(NCc2ncccc2Cl)Nc2c(/C=C/c3ccccc3F)cccc21] | 3-(((3-chloropyridin-2- yl)methyl)amino)-5-(2- fluorostyryl)-4H- benzo[e][1,2,4]thiadiazine 1,1- dioxide |
| 14-1 | 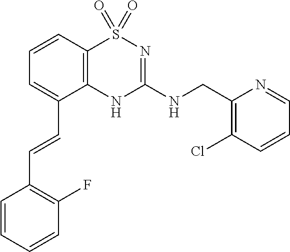 [SMILES: O=S1(=O)N=C(NCc2ncccc2Cl)Nc2c(/C=C/c3ccccc3F)cccc21] | (E)-3-(((3-chloropyridin-2- yl)methyl)amino)-5-(2- fluorostyryl)-4H- benzo[e][1,2,4]thiadiazine 1,1- dioxide |
| 14-1 (Z) | 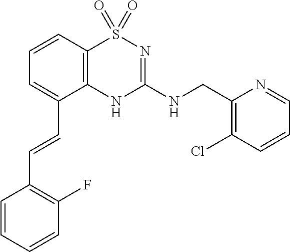 [SMILES: O=S1(=O)N=C(NCc2ncccc2Cl)Nc2c(/C=C/c3ccccc3F)cccc21] | (Z)-3-(((3-chloropyridin-2- yl)methyl)amino)-5-(2- fluorostyryl)-4H- benzo[e][1,2,4]thiadiazine 1,1- dioxide |
| 14-2 (racemic) | 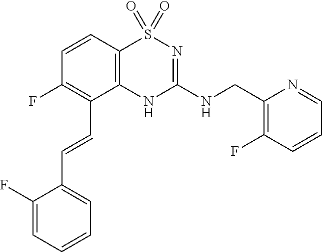 [SMILES: O=S1(=O)N=C(NCc2ncccc2F)Nc2c1ccc(F)c2/C=C/c1ccccc1F] | 6-fluoro-3-(((3-fluoropyridin-2- yl)methyl)amino)-5-(2- fluorostyryl)-4H- benzo[e][1,2,4]thiadiazine 1,1- dioxide |
| 14-2 | 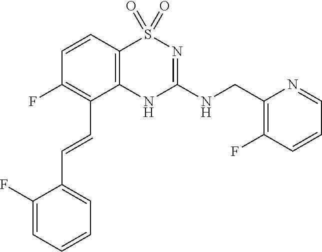 [SMILES: O=S1(=O)N=C(NCc2ncccc2F)Nc2c1ccc(F)c2/C=C/c1ccccc1F] | (E)-6-fluoro-3-(((3-fluoropyridin- 2-yl)methyl)amino)-5-(2- fluorostyryl)-4H- benzo[e][1,2,4]thiadiazine 1,1- dioxide |
| 14-2 (Z) | 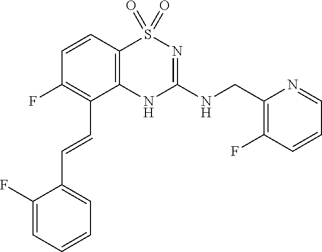 [SMILES: O=S1(=O)N=C(NCc2ncccc2F)Nc2c1ccc(F)c2/C=C/c1ccccc1F] | (Z)-6-fluoro-3-(((3-fluoropyridin- 2-yl)methyl)amino)-5-(2- fluorostyryl)-4H- benzo[e][1,2,4]thiadiazine 1,1- dioxide |
| 14-3 (racemic) | 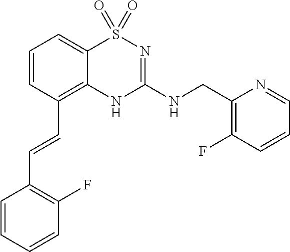 [SMILES: O=S1(=O)N=C(NCc2ncccc2F)Nc2c(/C=C/c3ccccc3F)cccc21] | 3-(((3-fluoropyridin-2- yl)methyl)amino)-5-(2- fluorostyryl)-4H- benzo[e][1,2,4]thiadiazine 1,1- dioxide |
| 14-3 | 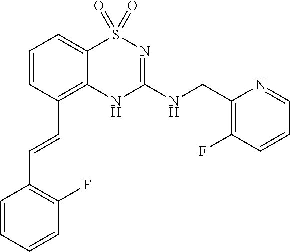 [SMILES: O=S1(=O)N=C(NCc2ncccc2F)Nc2c(/C=C/c3ccccc3F)cccc21] | (E)-3-(((3-fluoropyridin-2- yl)methyl)amino)-5-(2- fluorostyryl)-4H- benzo[e][1,2,4]thiadiazine 1,1- dioxide |
| 14-3 (Z) | 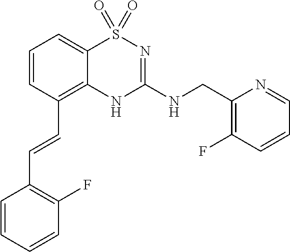 [SMILES: O=S1(=O)N=C(NCc2ncccc2F)Nc2c(/C=C/c3ccccc3F)cccc21] | (Z)-3-(((3-fluoropyridin-2- yl)methyl)amino)-5-(2- fluorostyryl)-4H- benzo[e][1,2,4]thiadiazine 1,1- dioxide |
| 15-1 | 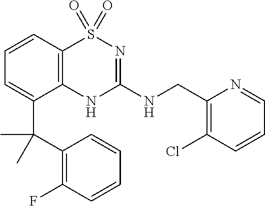 [SMILES: CC(C)(c1ccccc1F)c1cccc2c1NC(NCc1ncccc1Cl)=NS2(=O)=O] | 3-(((3-chloropyridin-2- yl)methyl)amino)-5-(2-(2- fluorophenyl)propan-2-yl)-4H- benzo[e][1,2,4]thiadiazine 1,1- dioxide |
| 16-1 | 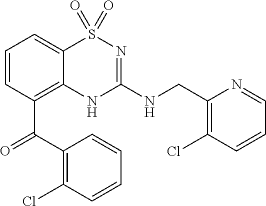 [SMILES: O=C(c1ccccc1Cl)c1cccc2c1NC(NCc1ncccc1Cl)=NS2(=O)=O] | (2-chlorophenyl)(3-(((3- chloropyridin-2-yl)methyl)amino)- 1,1-dioxido-4H- benzo[e][1,2,4]thiadiazin-5- yl)methanone |
| 16-2 | 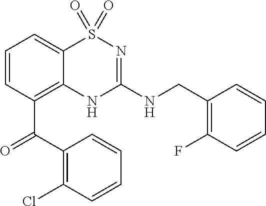 [SMILES: O=C(c1ccccc1Cl)c1cccc2c1NC(NCc1ccccc1F)=NS2(=O)=O] | (2-chlorophenyl)(3-((2- fluorobenzyl)amino)-1,1-dioxido- 4H-benzo[e][1,2,4]thiadiazin-5- yl)methanone |
| 16-3 | 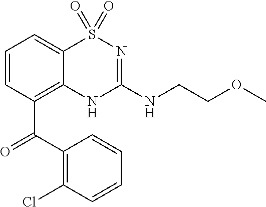 [SMILES: COCCNC1=NS(=O)(=O)c2cccc(C(=O)c3ccccc3Cl)c2N1] | (2-chlorophenyl)(3-((2- methoxyethyl)amino)-1,1-dioxido- 4H-benzo[e][1,2,4]thiadiazin-5- yl)methanone |
| 16-4 | 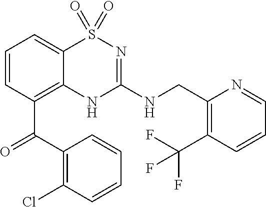 [SMILES: O=C(c1ccccc1Cl)c1cccc2c1NC(NCc1ncccc1C(F)(F)F)=NS2(=O)=O] | (2-chlorophenyl)(1,1-dioxido-3- (((3-(trifluoromethyl)pyridin-2- yl)methyl)amino)-4H- benzo[e][1,2,4]thiadiazin-5- yl)methanone |
| 16-5 | 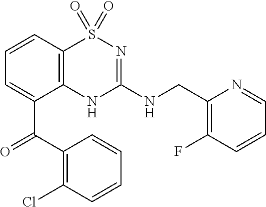 [SMILES: O=C(c1ccccc1Cl)c1cccc2c1NC(NCc1ncccc1F)=NS2(=O)=O] | (2-chlorophenyl)(3-(((3- fluoropyridin-2-yl)methyl)amino)- 1,1-dioxido-4H- benzo[e][1,2,4]thiadiazin-5- yl)methanone |
| 16-6 | 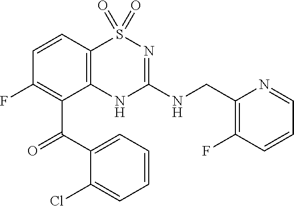 [SMILES: O=C(c1ccccc1Cl)c1c(F)ccc2c1NC(NCc1ncccc1F)=NS2(=O)=O] | (2-chlorophenyl)(6-fluoro-3-(((3- fluoropyridin-2-yl)methyl)amino)- 1,1-dioxido-4H- benzo[e][1,2,4]thiadiazin-5- yl)methanone |
| 17-1 | 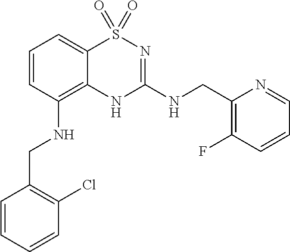 [SMILES: O=S1(=O)N=C(NCc2ncccc2F)Nc2c(NCc3ccccc3Cl)cccc21] | 5-((2-chlorobenzyl)amino)-3-(((3- fluoropyridin-2-yl)methyl)amino)- 4H-benzo[e][1,2,4]thiadiazine 1,1- dioxide |
| 18-1 | 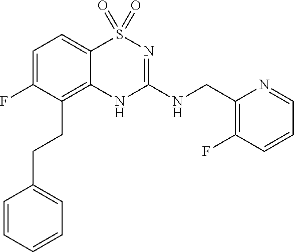 [SMILES: O=S1(=O)N=C(NCc2ncccc2F)Nc2c1ccc(F)c2CCc1ccccc1] | 6-fluoro-3-(((3-fluoropyridin-2- yl)methyl)amino)-5-phenethyl-4H- benzo[e][1,2,4]thiadiazine 1,1- dioxide |
| 19-1 | 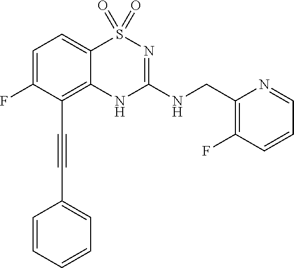 [SMILES: O=S1(=O)N=C(NCc2ncccc2F)Nc2c1ccc(F)c2C#Cc1ccccc1] | 6-fluoro-3-(((3-fluoropyridin-2- yl)methyl)amino)-5- (phenylethynyl)-4H- benzo[e][1,2,4]thiadiazine 1,1- dioxide |
| 20-1 | 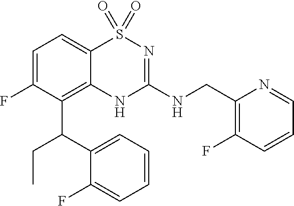 [SMILES: CCC(c1ccccc1F)c1c(F)ccc2c1NC(NCc1ncccc1F)=NS2(=O)=O] | 6-fluoro-5-(1-(2- fluorophenyl)propyl)-3-(((3- fluoropyridin-2-yl)methyl)amino)- 4H-benzo[e][1,2,4]thiadiazine 1,1- dioxide |
| 20-1 (S) | 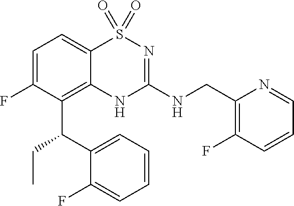 [SMILES: CC[C@@H](c1ccccc1F)c1c(F)ccc2c1NC(NCc1ncccc1F)=NS2(=O)=O] | (S)-6-fluoro-5-(1-(2- fluorophenyl)propyl)-3-(((3- fluoropyridin-2-yl)methyl)amino)- 4H-benzo[e][1,2,4]thiadiazine 1,1- dioxide |
| 20-1 (R) | 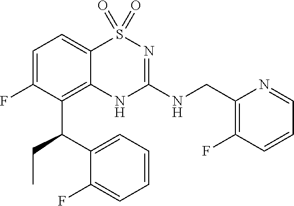 [SMILES: CC[C@H](c1ccccc1F)c1c(F)ccc2c1NC(NCc1ncccc1F)=NS2(=O)=O] | (R)-6-fluoro-5-(1-(2- fluorophenyl)propyl)-3-(((3- fluoropyridin-2-yl)methyl)amino)- 4H-benzo[e][1,2,4]thiadiazine 1,1- dioxide |
| 21-1 | 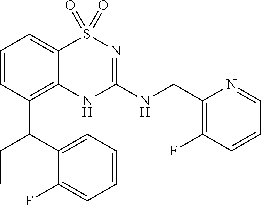 [SMILES: CCC(c1ccccc1F)c1cccc2c1NC(NCc1ncccc1F)=NS2(=O)=O] | 5-(1-(2-fluorophenyl)propyl)-3- (((3-fluoropyridin-2- yl)methyl)amino)-4H- benzo[e][1,2,4]thiadiazine 1,1- dioxide |
| 21-1 (S) | 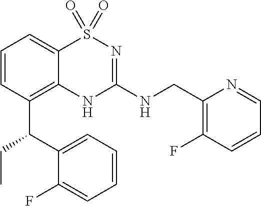 [SMILES: CC[C@@H](c1ccccc1F)c1cccc2c1NC(NCc1ncccc1F)=NS2(=O)=O] | (S)- 5-(1-(2-fluorophenyl)propyl)- 3-(((3-fluoropyridin-2- yl)methyl)amino)-4H- benzo[e][1,2,4]thiadiazine 1,1- dioxide |
| 21-1 (R) | 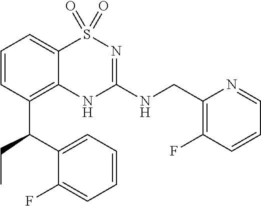 [SMILES: CC[C@H](c1ccccc1F)c1cccc2c1NC(NCc1ncccc1F)=NS2(=O)=O] | (R)-5-(1-(2-fluorophenyl)propyl)- 3-(((3-fluoropyridin-2- yl)methyl)amino)-4H- benzo[e][1,2,4]thiadiazine 1,1- dioxide |
| 22-1 | 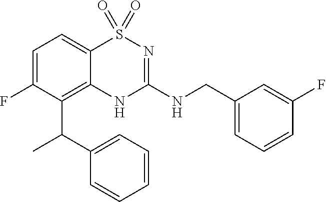 [SMILES: CC(c1ccccc1)c1c(F)ccc2c1NC(NCc1cccc(F)c1)=NS2(=O)=O] | 6-fluoro-3-((3- fluorobenzyl)amino)-5-(1- phenylethyl)-4H- benzo[e][1,2,4]thiadiazine 1,1- dioxide |
| 22-1 (S) | 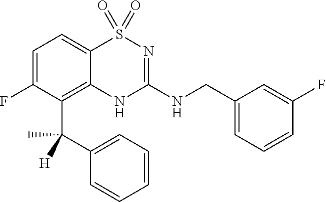 [SMILES: C[C@@H](c1ccccc1)c1c(F)ccc2c1NC(NCc1cccc(F)c1)=NS2(=O)=O] | (S)- 6-fluoro-3-((3- fluorobenzyl)amino)-5-(1- phenylethyl)-4H- benzo[e][1,2,4]thiadiazine 1,1- dioxide |
| 22-1 (R) | 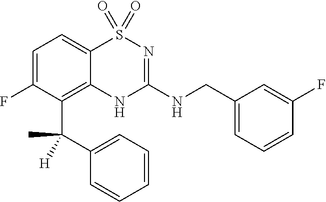 [SMILES: C[C@H](c1ccccc1)c1c(F)ccc2c1NC(NCc1cccc(F)c1)=NS2(=O)=O] | (R)-6-fluoro-3-((3- fluorobenzyl)amino)-5-(1- phenylethyl)-4H- benzo[e][1,2,4]thiadiazine 1,1- dioxide |
| 22-2 | 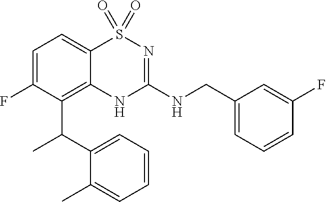 [SMILES: Cc1ccccc1C(C)c1c(F)ccc2c1NC(NCc1cccc(F)c1)=NS2(=O)=O] | 6-fluoro-3-((3- fluorobenzyl)amino)-5-(1-(o- tolyl)ethyl)-4H- benzo[e][1,2,4]thiadiazine 1,1- dioxide |
| 22-2 (S) | 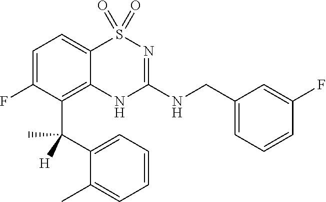 [SMILES: Cc1ccccc1[C@H](C)c1c(F)ccc2c1NC(NCc1cccc(F)c1)=NS2(=O)=O] | (S)-6-fluoro-3-((3- fluorobenzyl)amino)-5-(1-(o- tolyl)ethyl)-4H- benzo[e][1,2,4]thiadiazine 1,1- dioxide |
| 22-2 (R) | 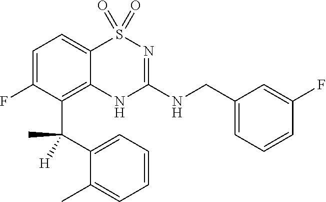 [SMILES: Cc1ccccc1[C@@H](C)c1c(F)ccc2c1NC(NCc1cccc(F)c1)=NS2(=O)=O] | (R)-6-fluoro-3-((3- fluorobenzyl)amino)-5-(1-(o- tolyl)ethyl)-4H- benzo[e][1,2,4]thiadiazine 1,1- dioxide |
| 22-3 | 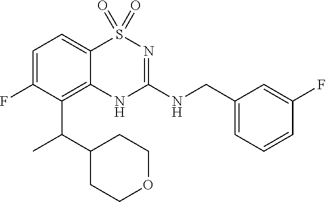 [SMILES: CC(c1c(F)ccc2c1NC(NCc1cccc(F)c1)=NS2(=O)=O)C1CCOCC1] | 6-fluoro-3-((3- fluorobenzyl)amino)-5-(1- (tetrahydro-2H-pyran-4-yl)ethyl)- 4H-benzo[e][1,2,4]thiadiazine 1,1- dioxide |
| 22-3 (S) | 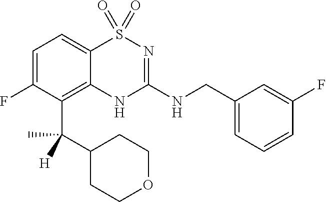 [SMILES: C[C@H](c1c(F)ccc2c1NC(NCc1cccc(F)c1)=NS2(=O)=O)C1CCOCC1] | (S)-6-fluoro-3-((3- fluorobenzyl)amino)-5-(1- (tetrahydro-2H-pyran-4-yl)ethyl)- 4H-benzo[e][1,2,4]thiadiazine 1,1- dioxide |
| 22-3 (R) | 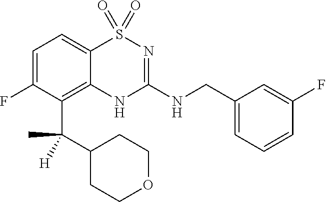 [SMILES: C[C@@H](c1c(F)ccc2c1NC(NCc1cccc(F)c1)=NS2(=O)=O)C1CCOCC1] | (R)-6-fluoro-3-((3- fluorobenzyl)amino)-5-(1- (tetrahydro-2H-pyran-4-yl)ethyl)- 4H-benzo[e][1,2,4]thiadiazine 1,1- dioxide |
| 22-4 | 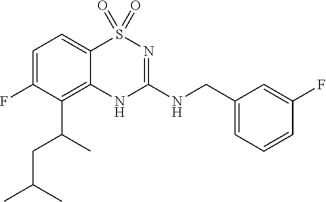 [SMILES: CC(C)CC(C)c1c(F)ccc2c1NC(NCc1cccc(F)c1)=NS2(=O)=O] | 6-fluoro-3-((3- fluorobenzyl)amino)-5-(4- methylpentan-2-yl)-4H- benzo[e][1,2,4]thiadiazine 1,1- dioxide |
| 22-4 (S) | 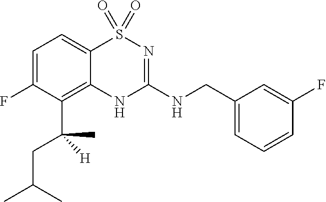 [SMILES: CC(C)C[C@H](C)c1c(F)ccc2c1NC(NCc1cccc(F)c1)=NS2(=O)=O] | (S)-6-fluoro-3-((3- fluorobenzyl)amino)-5-(4- methylpentan-2-yl)-4H- benzo[e][1,2,4]thiadiazine 1,1- dioxide |
| 22-4 (R) | 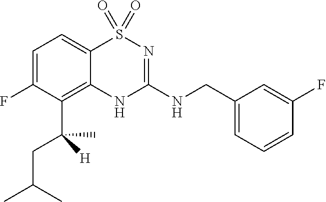 [SMILES: CC(C)C[C@@H](C)c1c(F)ccc2c1NC(NCc1cccc(F)c1)=NS2(=O)=O] | (R)-6-fluoro-3-((3- fluorobenzyl)amino)-5-(4- methylpentan-2-yl)-4H- benzo[e][1,2,4]thiadiazine 1,1- dioxide |
| 22-5 | 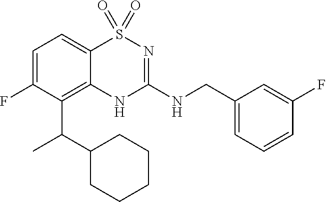 [SMILES: CC(c1c(F)ccc2c1NC(NCc1cccc(F)c1)=NS2(=O)=O)C1CCCCC1] | 5-(1-cyclohexylethyl)-6-fluoro-3- ((3-fluorobenzyl)amino)-4H- benzo[e][1,2,4]thiadiazine 1,1- dioxide |
| 22-5 (S) | 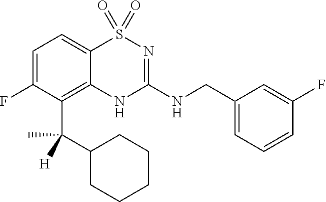 [SMILES: C[C@H](c1c(F)ccc2c1NC(NCc1cccc(F)c1)=NS2(=O)=O)C1CCCCC1] | (S)-5-(1-cyclohexylethyl)-6-fluoro- 3-((3-fluorobenzyl)amino)-4H- benzo[e][1,2,4]thiadiazine 1,1- dioxide |
| 22-5 (R) | 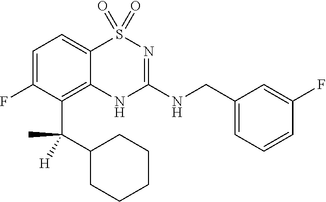 [SMILES: C[C@@H](c1c(F)ccc2c1NC(NCc1cccc(F)c1)=NS2(=O)=O)C1CCCCC1] | (R)-5-(1-cyclohexylethyl)-6- fluoro-3-((3-fluorobenzyl)amino)- 4H-benzo[e][1,2,4]thiadiazine 1,1- dioxide |
| 22-6 | 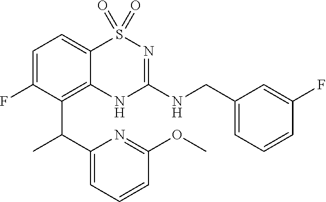 [SMILES: COc1cccc(C(C)c2c(F)ccc3c2NC(NCc2cccc(F)c2)=NS3(=O)=O)n1] | 6-fluoro-3-((3- fluorobenzyl)amino)-5-(1-(6- methoxypyridin-2-yl)ethyl)-4H- benzo[e][1,2,4]thiadiazine 1,1- dioxide |
| 22-6 (S) | 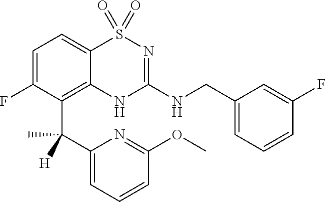 [SMILES: COc1cccc([C@H](C)c2c(F)ccc3c2NC(NCc2cccc(F)c2)=NS3(=O)=O)n1] | (S)-6-fluoro-3-((3- fluorobenzyl)amino)-5-(1-(6- methoxypyridin-2-yl)ethyl)-4H- benzo[e][1,2,4]thiadiazine 1,1- dioxide |
| 22-6 (R) | 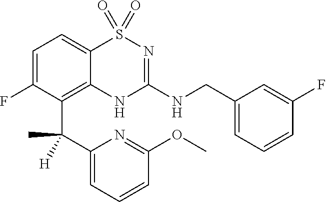 [SMILES: COc1cccc([C@@H](C)c2c(F)ccc3c2NC(NCc2cccc(F)c2)=NS3(=O)=O)n1] | (R)-6-fluoro-3-((3- fluorobenzyl)amino)-5-(1-(6- methoxypyridin-2-yl)ethyl)-4H- benzo[e][1,2,4]thiadiazine 1,1- dioxide |
| 22-7 | 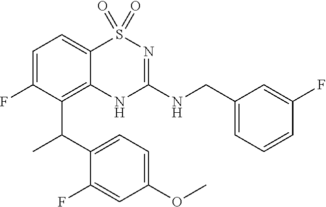 [SMILES: COc1ccc(C(C)c2c(F)ccc3c2NC(NCc2cccc(F)c2)=NS3(=O)=O)c(F)c1] | 6-fluoro-5-(1-(2-fluoro-4- methoxyphenyl)ethyl)-3-((3- fluorobenzyl)amino)-4H- benzo[e][1,2,4]thiadiazine 1,1- dioxide |
| 22-7 (S) | 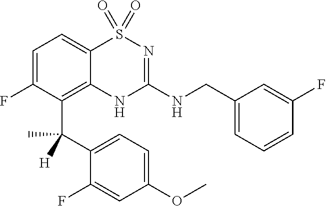 [SMILES: COc1ccc([C@H](C)c2c(F)ccc3c2NC(NCc2cccc(F)c2)=NS3(=O)=O)c(F)c1] | (S)-6-fluoro-5-(1-(2-fluoro-4- methoxyphenyl)ethyl)-3-((3- fluorobenzyl)amino)-4H- benzo[e][1,2,4]thiadiazine 1,1- dioxide |
| 22-7 (R) | 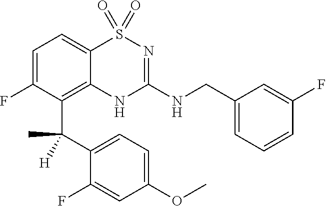 [SMILES: COc1ccc([C@@H](C)c2c(F)ccc3c2NC(NCc2cccc(F)c2)=NS3(=O)=O)c(F)c1] | (R)-6-fluoro-5-(1-(2-fluoro-4- methoxyphenyl)ethyl)-3-((3- fluorobenzyl)amino)-4H- benzo[e][1,2,4]thiadiazine 1,1- dioxide |
| 22-8 | 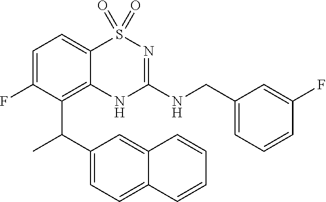 [SMILES: CC(c1ccc2ccccc2c1)c1c(F)ccc2c1NC(NCc1cccc(F)c1)=NS2(=O)=O] | 6-fluoro-3-((3- fluorobenzyl)amino)-5-(1- (naphthalen-2-yl)ethyl)-4H- benzo[e][1,2,4]thiadiazine 1,1- dioxide |
| 22-8 (S) | 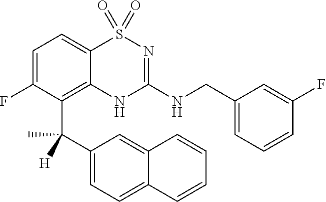 [SMILES: C[C@@H](c1ccc2ccccc2c1)c1c(F)ccc2c1NC(NCc1cccc(F)c1)=NS2(=O)=O] | (S)- 6-fluoro-3-((3- fluorobenzyl)amino)-5-(1- (naphthalen-2-yl)ethyl)-4H- benzo[e][1,2,4]thiadiazine 1,1- dioxide |
| 22-8 (R) | 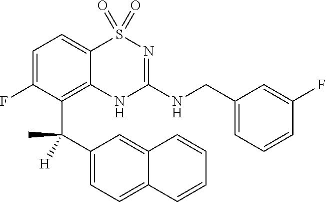 [SMILES: C[C@H](c1ccc2ccccc2c1)c1c(F)ccc2c1NC(NCc1cccc(F)c1)=NS2(=O)=O] | (R)-6-fluoro-3-((3- fluorobenzyl)amino)-5-(1- (naphthalen-2-yl)ethyl)-4H- benzo[e][1,2,4]thiadiazine 1,1- dioxide |
| 22-9 | 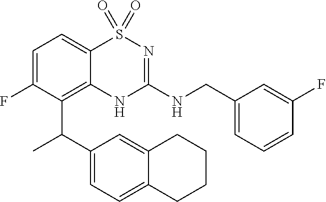 [SMILES: CC(c1ccc2c(c1)CCCC2)c1c(F)ccc2c1NC(NCc1cccc(F)c1)=NS2(=O)=O] | 6-fluoro-3-((3- fluorobenzyl)amino)-5-(1-(5,6,7,8- tetrahydronaphthalen-2-yl)ethyl)- 4H-benzo[e][1,2,4]thiadiazine 1,1- dioxide |
| 22-9 (S) | 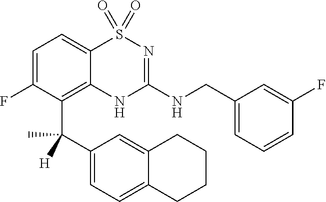 [SMILES: C[C@@H](c1ccc2c(c1)CCCC2)c1c(F)ccc2c1NC(NCc1cccc(F)c1)=NS2(=O)=O] | (S)-6-fluoro-3-((3- fluorobenzyl)amino)-5-(1-(5,6,7,8- tetrahydronaphthalen-2-yl)ethyl)- 4H-benzo[e][1,2,4]thiadiazine 1,1- dioxide |
| 22-9 (R) | 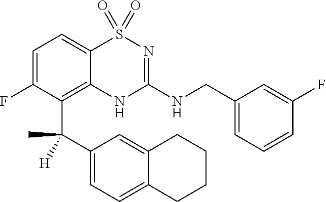 [SMILES: C[C@H](c1ccc2c(c1)CCCC2)c1c(F)ccc2c1NC(NCc1cccc(F)c1)=NS2(=O)=O] | (R)-6-fluoro-3-((3- fluorobenzyl)amino)-5-(1-(5,6,7,8- tetrahydronaphthalen-2-yl)ethyl)- 4H-benzo[e][1,2,4]thiadiazine 1,1- dioxide |
| 22-10 | 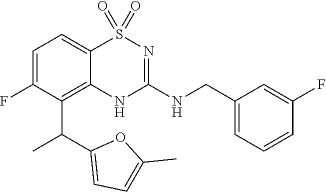 [SMILES: Cc1ccc(C(C)c2c(F)ccc3c2NC(NCc2cccc(F)c2)=NS3(=O)=O)o1] | 6-fluoro-3-((3- fluorobenzyl)amino)-5-(1-(5- methylfuran-2-yl)ethyl)-4H- benzo[e][1,2,4]thiadiazine 1,1- dioxide |
| 22-10 (S) | 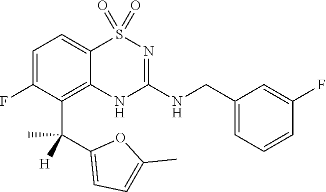 [SMILES: Cc1ccc([C@H](C)c2c(F)ccc3c2NC(NCc2cccc(F)c2)=NS3(=O)=O)o1] | (S)-6-fluoro-3-((3- fluorobenzyl)amino)-5-(1-(5- methylfuran-2-yl)ethyl)-4H- benzo[e][1,2,4]thiadiazine 1,1- dioxide |
| 23-10 (R) | 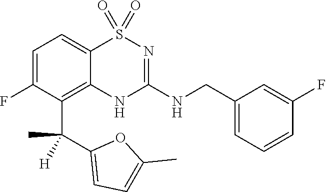 [SMILES: Cc1ccc([C@@H](C)c2c(F)ccc3c2NC(NCc2cccc(F)c2)=NS3(=O)=O)o1] | (R)-6-fluoro-3-((3- fluorobenzyl)amino)-5-(1-(5- methylfuran-2-yl)ethyl)-4H- benzo[e][1,2,4]thiadiazine 1,1- dioxide |
| 22-11 | 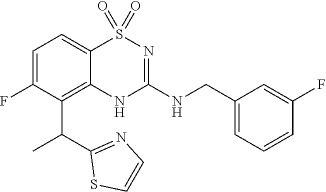 [SMILES: CC(c1nccs1)c1c(F)ccc2c1NC(NCc1cccc(F)c1)=NS2(=O)=O] | 6-fluoro-3-((3- fluorobenzyl)amino)-5-(1-(thiazol- 2-yl)ethyl)-4H- benzo[e][1,2,4]thiadiazine 1,1- dioxide |
| 22-11 (S) | 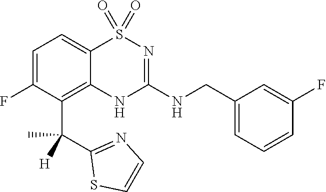 [SMILES: C[C@@H](c1nccs1)c1c(F)ccc2c1NC(NCc1cccc(F)c1)=NS2(=O)=O] | (S)-6-fluoro-3-((3- fluorobenzyl)amino)-5-(1-(thiazol- 2-yl)ethyl)-4H- benzo[e][1,2,4]thiadiazine 1,1- dioxide |
| 22-11 (R) | 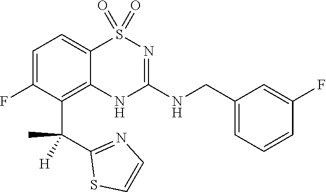 [SMILES: C[C@H](c1nccs1)c1c(F)ccc2c1NC(NCc1cccc(F)c1)=NS2(=O)=O] | (R)-6-fluoro-3-((3- fluorobenzyl)amino)-5-(1-(thiazol- 2-yl)ethyl)-4H- benzo[e][1,2,4]thiadiazine 1,1- dioxide |
| 22-12 | 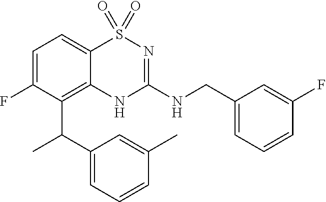 [SMILES: Cc1cccc(C(C)c2c(F)ccc3c2NC(NCc2cccc(F)c2)=NS3(=O)=O)c1] | 6-fluoro-3-((3- fluorobenzyl)amino)-5-(1-(m- tolyl)ethyl)-4H- benzo[e][1,2,4]thiadiazine 1,1- dioxide |
| 22-12 (S) | 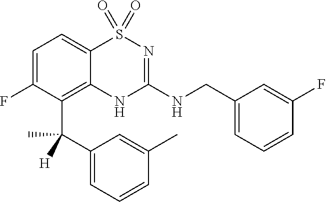 [SMILES: Cc1cccc([C@H](C)c2c(F)ccc3c2NC(NCc2cccc(F)c2)=NS3(=O)=O)c1] | (S)-6-fluoro-3-((3- fluorobenzyl)amino)-5-(1-(m- tolyl)ethyl)-4H- benzo[e][1,2,4]thiadiazine 1,1- dioxide |
| 22-12 (R) | 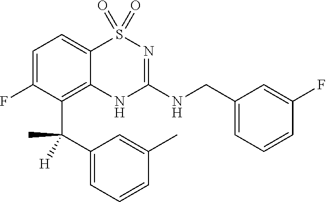 [SMILES: Cc1cccc([C@@H](C)c2c(F)ccc3c2NC(NCc2cccc(F)c2)=NS3(=O)=O)c1] | (R)-6-fluoro-3-((3- fluorobenzyl)amino)-5-(1-(m- tolyl)ethyl)-4H- benzo[e][1,2,4]thiadiazine 1,1- dioxide |
| 22-13 | 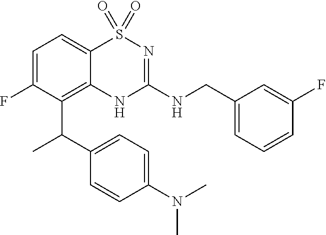 [SMILES: CC(c1ccc(N(C)C)cc1)c1c(F)ccc2c1NC(NCc1cccc(F)c1)=NS2(=O)=O] | 5-(1-(4- (dimethylamino)phenyl)ethyl)-6- fluoro-3-((3-fluorobenzyl)amino)- 4H-benzo[e][1,2,4]thiadiazine 1,1- dioxide |
| 22-13 (S) | 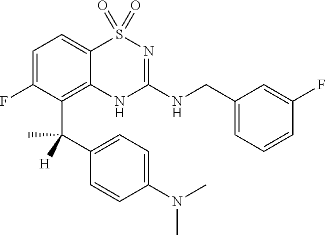 [SMILES: C[C@@H](c1ccc(N(C)C)cc1)c1c(F)ccc2c1NC(NCc1cccc(F)c1)=NS2(=O)=O] | (S)- 5-(1-(4- (dimethylamino)phenyl)ethyl)-6- fluoro-3-((3-fluorobenzyl)amino)- 4H-benzo[e][1,2,4]thiadiazine 1,1- dioxide |
| 22-13 (R) | 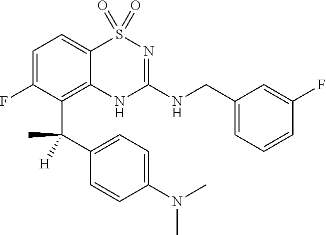 [SMILES: C[C@H](c1ccc(N(C)C)cc1)c1c(F)ccc2c1NC(NCc1cccc(F)c1)=NS2(=O)=O] | (R)-5-(1-(4- (dimethylamino)phenyl)ethyl)-6- fluoro-3-((3-fluorobenzyl)amino)- 4H-benzo[e][1,2,4]thiadiazine 1,1- dioxide |
| 22-14 | 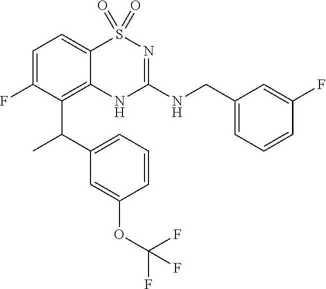 [SMILES: CC(c1cccc(OC(F)(F)F)c1)c1c(F)ccc2c1NC(NCc1cccc(F)c1)=NS2(=O)=O] | 6-fluoro-3-((3- fluorobenzyl)amino)-5-(1-(3- (trifluoromethoxy)phenyl)ethyl)- 4H-benzo[e][1,2,4]thiadiazine 1,1- dioxide |
| 22-14 (S) | 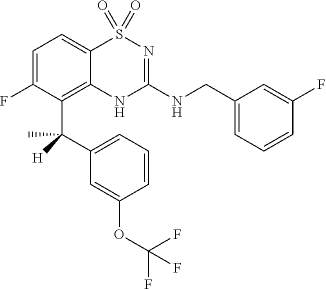 [SMILES: C[C@@H](c1cccc(OC(F)(F)F)c1)c1c(F)ccc2c1NC(NCc1cccc(F)c1)=NS2(=O)=O] | (S)-6-fluoro-3-((3- fluorobenzyl)amino)-5-(1-(3- (trifluoromethoxy)phenyl)ethyl)- 4H-benzo[e][1,2,4]thiadiazine 1,1- dioxide |
| 22-14 (R) | 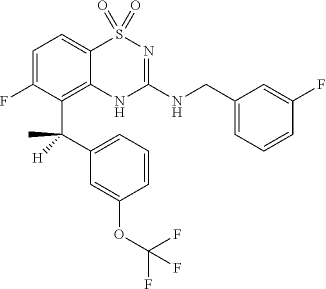 [SMILES: C[C@H](c1cccc(OC(F)(F)F)c1)c1c(F)ccc2c1NC(NCc1cccc(F)c1)=NS2(=O)=O] | (R)-6-fluoro-3-((3- fluorobenzyl)amino)-5-(1-(3- (trifluoromethoxy)phenyl)ethyl)- 4H-benzo[e][1,2,4]thiadiazine 1,1- dioxide |
| 22-15 | 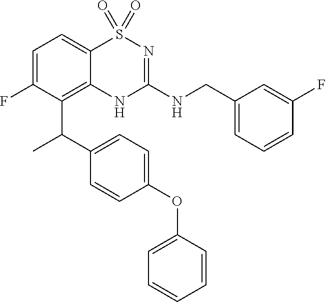 [SMILES: CC(c1ccc(Oc2ccccc2)cc1)c1c(F)ccc2c1NC(NCc1cccc(F)c1)=NS2(=O)=O] | 6-fluoro-3-((3- fluorobenzyl)amino)-5-(1-(4- phenoxyphenyl)ethyl)-4H- benzo[e][1,2,4]thiadiazine 1,1- dioxide |
| 22-15 (S) | 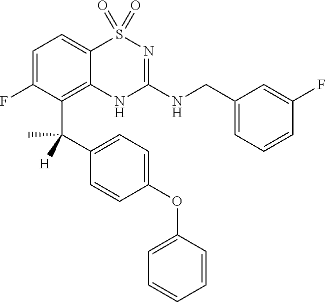 [SMILES: C[C@@H](c1ccc(Oc2ccccc2)cc1)c1c(F)ccc2c1NC(NCc1cccc(F)c1)=NS2(=O)=O] | (S)-6-fluoro-3-((3- fluorobenzyl)amino)-5-(1-(4- phenoxyphenyl)ethyl)-4H- benzo[e][1,2,4]thiadiazine 1,1- dioxide |
| 22-15 (R) | 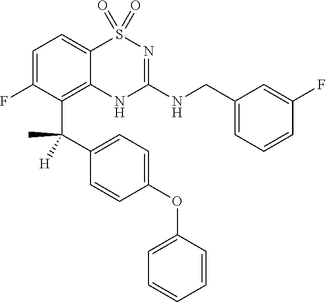 [SMILES: C[C@H](c1ccc(Oc2ccccc2)cc1)c1c(F)ccc2c1NC(NCc1cccc(F)c1)=NS2(=O)=O] | (R)-6-fluoro-3-((3- fluorobenzyl)amino)-5-(1-(4- phenoxyphenyl)ethyl)-4H- benzo[e][1,2,4]thiadiazine 1,1- dioxide |
| 22-16 | 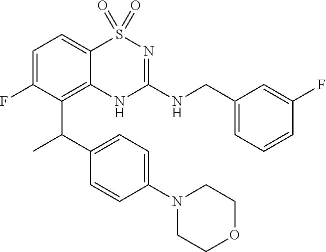 [SMILES: CC(c1ccc(N2CCOCC2)cc1)c1c(F)ccc2c1NC(NCc1cccc(F)c1)=NS2(=O)=O] | 6-fluoro-3-((3- fluorobenzyl)amino)-5-(1-(4- morpholinophenyl)ethyl)-4H- benzo[e][1,2,4]thiadiazine 1,1- dioxide |
| 22-16 (S) | 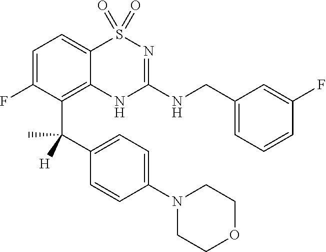 [SMILES: C[C@@H](c1ccc(N2CCOCC2)cc1)c1c(F)ccc2c1NC(NCc1cccc(F)c1)=NS2(=O)=O] | (S)-6-fluoro-3-((3- fluorobenzyl)amino)-5-(1-(4- morpholinophenyl)ethyl)-4H- benzo[e][1,2,4]thiadiazine 1,1- dioxide |
| 22-16 (R) | 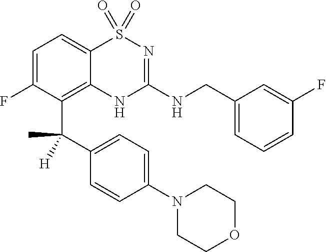 [SMILES: C[C@H](c1ccc(N2CCOCC2)cc1)c1c(F)ccc2c1NC(NCc1cccc(F)c1)=NS2(=O)=O] | (R)-6-fluoro-3-((3- fluorobenzyl)amino)-5-(1-(4- morpholinophenyl)ethyl)-4H- benzo[e][1,2,4]thiadiazine 1,1- dioxide |
| 22-17 | 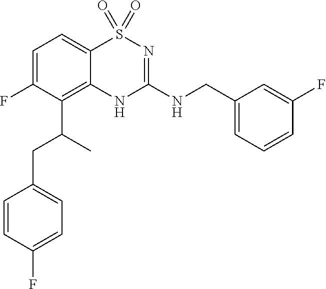 [SMILES: CC(Cc1ccc(F)cc1)c1c(F)ccc2c1NC(NCc1cccc(F)c1)=NS2(=O)=O] | 6-fluoro-3-((3- fluorobenzyl)amino)-5-(1-(4- fluorophenyl)propan-2-yl)-4H- benzo[e][1,2,4]thiadiazine 1,1- dioxide |
| 22-17 (S) | 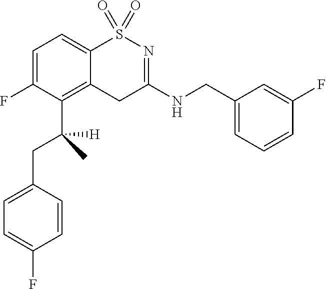 [SMILES: C[C@@H](Cc1ccc(F)cc1)c1c(F)ccc2c1CC(NCc1cccc(F)c1)=NS2(=O)=O] | (S)-6-fluoro-3-((3- fluorobenzyl)amino)-5-(1-(4- fluorophenyl)propan-2-yl)-4H- benzo[e][1,2,4]thiadiazine 1,1- dioxide |
| 22-17 (R) | 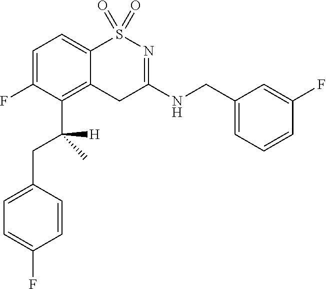 [SMILES: C[C@H](Cc1ccc(F)cc1)c1c(F)ccc2c1CC(NCc1cccc(F)c1)=NS2(=O)=O] | (R)-6-fluoro-3-((3- fluorobenzyl)amino)-5-(1-(4- fluorophenyl)propan-2-yl)-4H- benzo[e][1,2,4]thiadiazine 1,1- dioxide |
| 22-18 | 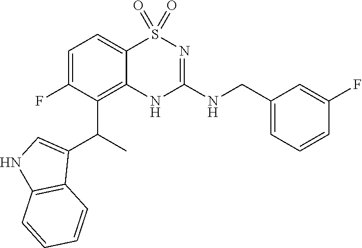 [SMILES: CC(c1c(F)ccc2c1NC(NCc1cccc(F)c1)=NS2(=O)=O)c1c[nH]c2ccccc12] | 5-(1-(1H-indol-3-yl)ethyl)-6- fluoro-3-((3-fluorobenzyl)amino)- 4H-benzo[e][1,2,4]thiadiazine 1,1- dioxide |
| 22-18 (S) | 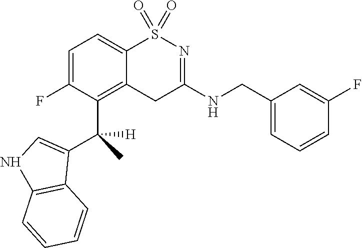 [SMILES: C[C@H](c1c(F)ccc2c1CC(NCc1cccc(F)c1)=NS2(=O)=O)c1c[nH]c2ccccc12] | (S)-5-(1-(1H-indol-3-yl)ethyl)-6- fluoro-3-((3-fluorobenzyl)amino)- 4H-benzo[e][1,2,4]thiadiazine 1,1- dioxide |
| 22-18 (R) | 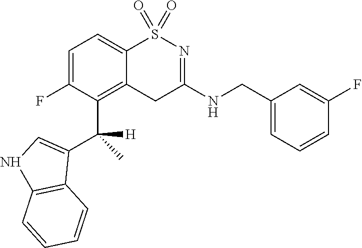 [SMILES: C[C@@H](c1c(F)ccc2c1CC(NCc1cccc(F)c1)=NS2(=O)=O)c1c[nH]c2ccccc12] | (R)-5-(1-(1H-indol-3-yl)ethyl)-6- fluoro-3-((3-fluorobenzyl)amino)- 4H-benzo[e][1,2,4]thiadiazine 1,1- dioxide |
| 22-19 | 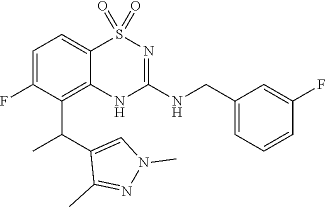 [SMILES: Cc1nn(C)cc1C(C)c1c(F)ccc2c1NC(NCc1cccc(F)c1)=NS2(=O)=O] | 5-(1-(1,3-dimethyl-1H-pyrazol-4- yl)ethyl)-6-fluoro-3-((3- fluorobenzyl)amino)-4H- benzo[e][1,2,4]thiadiazine 1,1- dioxide |
| 22-19 (S) | 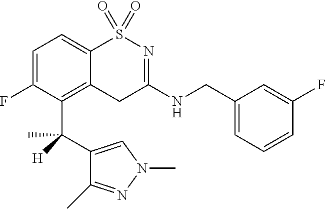 [SMILES: Cc1nn(C)cc1[C@H](C)c1c(F)ccc2c1CC(NCc1cccc(F)c1)=NS2(=O)=O] | (S)-5-(1-(1,3-dimethyl-1H-pyrazol- 4-yl)ethyl)-6-fluoro-3-((3- fluorobenzyl)amino)-4H- benzo[e][1,2,4]thiadiazine 1,1- dioxide |
| 22-19 (R) | 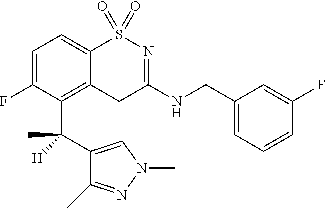 [SMILES: Cc1nn(C)cc1[C@@H](C)c1c(F)ccc2c1CC(NCc1cccc(F)c1)=NS2(=O)=O] | (R)-5-(1-(1,3-dimethyl-1H- pyrazol-4-yl)ethyl)-6-fluoro-3-((3- fluorobenzyl)amino)-4H- benzo[e][1,2,4]thiadiazine 1,1- dioxide |
| 22-20 | 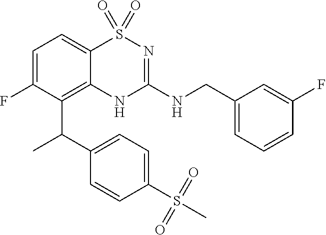 [SMILES: CC(c1ccc(S(C)(=O)=O)cc1)c1c(F)ccc2c1NC(NCc1cccc(F)c1)=NS2(=O)=O] | 6-fluoro-3-((3- fluorobenzyl)amino)-5-(1-(4- (methylsulfonyl)phenyl)ethyl)-4H- benzo[e][1,2,4]thiadiazine 1,1- dioxide |
| 22-20 (S) | 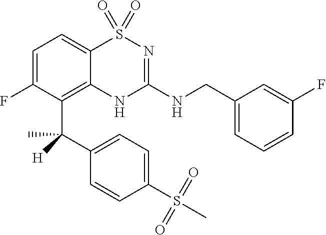 [SMILES: C[C@@H](c1ccc(S(C)(=O)=O)cc1)c1c(F)ccc2c1NC(NCc1cccc(F)c1)=NS2(=O)=O] | (S)-6-fluoro-3-((3- fluorobenzyl)amino)-5-(1-(4- (methylsulfonyl)phenyl)ethyl)-4H- benzo[e][1,2,4]thiadiazine 1,1- dioxide |
| 22-20 (R) | 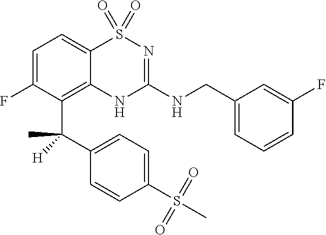 [SMILES: C[C@H](c1ccc(S(C)(=O)=O)cc1)c1c(F)ccc2c1NC(NCc1cccc(F)c1)=NS2(=O)=O] | (R)-6-fluoro-3-((3- fluorobenzyl)amino)-5-(1-(4- (methylsulfonyl)phenyl)ethyl)-4H- benzo[e][1,2,4]thiadiazine 1,1- dioxide |
| 22-21 | 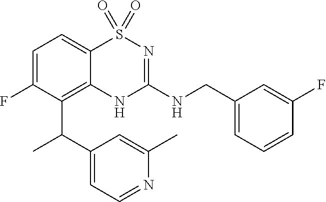 [SMILES: Cc1cc(C(C)c2c(F)ccc3c2NC(NCc2cccc(F)c2)=NS3(=O)=O)ccn1] | 6-fluoro-3-((3- fluorobenzyl)amino)-5-(1-(2- methylpyridin-4-yl)ethyl)-4H- benzo[e][1,2,4]thiadiazine 1,1- dioxide |
| 22-21 (S) | 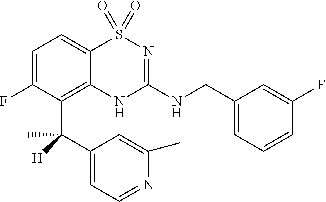 [SMILES: Cc1cc([C@H](C)c2c(F)ccc3c2NC(NCc2cccc(F)c2)=NS3(=O)=O)ccn1] | (S)-6-fluoro-3-((3- fluorobenzyl)amino)-5-(1-(2- methylpyridin-4-yl)ethyl)-4H- benzo[e][1,2,4]thiadiazine 1,1- dioxide |
| 22-21 (R) | 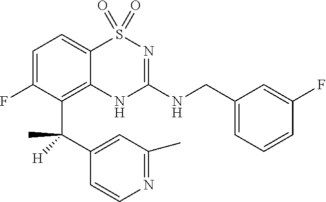 [SMILES: Cc1cc([C@@H](C)c2c(F)ccc3c2NC(NCc2cccc(F)c2)=NS3(=O)=O)ccn1] | (R)-6-fluoro-3-((3- fluorobenzyl)amino)-5-(1-(2- methylpyridin-4-yl)ethyl)-4H- benzo[e][1,2,4]thiadiazine 1,1- dioxide |
| 22-22 | 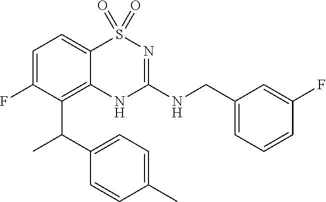 [SMILES: Cc1ccc(C(C)c2c(F)ccc3c2NC(NCc2cccc(F)c2)=NS3(=O)=O)cc1] | 6-fluoro-3-((3- fluorobenzyl)amino)-5-(1-(p- tolyl)ethyl)-4H- benzo[e][1,2,4]thiadiazine 1,1- dioxide |
| 22-22 (S) | 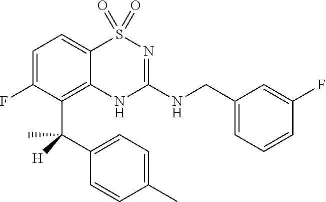 [SMILES: Cc1ccc([C@H](C)c2c(F)ccc3c2NC(NCc2cccc(F)c2)=NS3(=O)=O)cc1] | (S)-6-fluoro-3-((3- fluorobenzyl)amino)-5-(1-(p- tolyl)ethyl)-4H- benzo[e][1,2,4]thiadiazine 1,1- dioxide |
| 22-22 (R) | 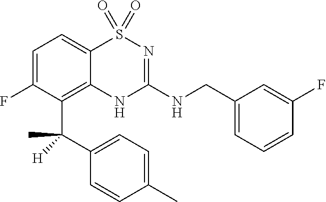 [SMILES: Cc1ccc([C@@H](C)c2c(F)ccc3c2NC(NCc2cccc(F)c2)=NS3(=O)=O)cc1] | (R)-6-fluoro-3-((3- fluorobenzyl)amino)-5-(1-(p- tolyl)ethyl)-4H- benzo[e][1,2,4]thiadiazine 1,1- dioxide |
| 22-23 | 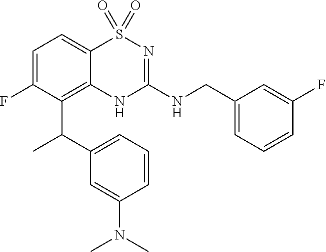 [SMILES: CC(c1cccc(N(C)C)c1)c1c(F)ccc2c1NC(NCc1cccc(F)c1)=NS2(=O)=O] | 5-(1-(3- (dimethylamino)phenyl)ethyl)-6- fluoro-3-((3-fluorobenzyl)amino)- 4H-benzo[e][1,2,4]thiadiazine 1,1- dioxide |
| 22-23 (S) | 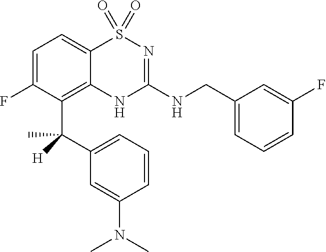 [SMILES: C[C@@H](c1cccc(N(C)C)c1)c1c(F)ccc2c1NC(NCc1cccc(F)c1)=NS2(=O)=O] | (S)-5-(1-(3- (dimethylamino)phenyl)ethyl)-6- fluoro-3-((3-fluorobenzyl)amino)- 4H-benzo[e][1,2,4]thiadiazine 1,1- dioxide |
| 22-23 (R) | 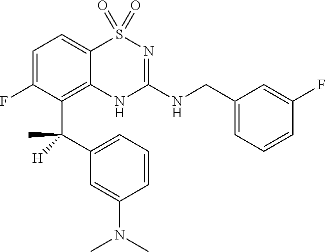 [SMILES: C[C@H](c1cccc(N(C)C)c1)c1c(F)ccc2c1NC(NCc1cccc(F)c1)=NS2(=O)=O] | (R)-5-(1-(3- (dimethylamino)phenyl)ethyl)-6- fluoro-3-((3-fluorobenzyl)amino)- 4H-benzo[e][1,2,4]thiadiazine 1,1- dioxide |
| 22-24 | 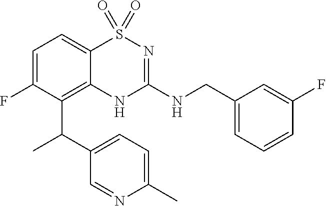 [SMILES: Cc1ccc(C(C)c2c(F)ccc3c2NC(NCc2cccc(F)c2)=NS3(=O)=O)cn1] | 6-fluoro-3-((3- fluorobenzyl)amino)-5-(1-(6- methylpyridin-3-yl)ethyl)-4H- benzo[e][1,2,4]thiadiazine 1,1- dioxide |
| 22-24 (S) | 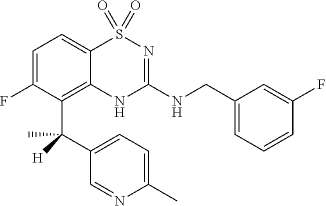 [SMILES: Cc1ccc([C@H](C)c2c(F)ccc3c2NC(NCc2cccc(F)c2)=NS3(=O)=O)cn1] | (S)-6-fluoro-3-((3- fluorobenzyl)amino)-5-(1-(6- methylpyridin-3-yl)ethyl)-4H- benzo[e][1,2,4]thiadiazine 1,1- dioxide |
| 22-24 (R) | 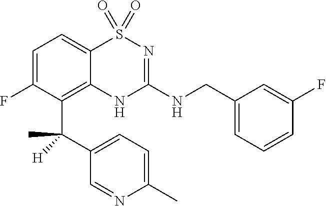 [SMILES: Cc1ccc([C@@H](C)c2c(F)ccc3c2NC(NCc2cccc(F)c2)=NS3(=O)=O)cn1] | (R)-6-fluoro-3-((3- fluorobenzyl)amino)-5-(1-(6- methylpyridin-3-yl)ethyl)-4H- benzo[e][1,2,4]thiadiazine 1,1- dioxide |
| 22-25 | 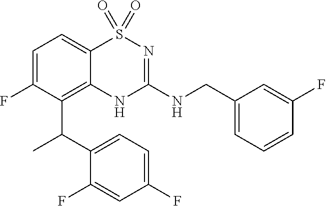 [SMILES: CC(c1ccc(F)cc1F)c1c(F)ccc2c1NC(NCc1cccc(F)c1)=NS2(=O)=O] | 5-(1-(2,4-difluorophenyl)ethyl)-6- fluoro-3-((3-fluorobenzyl)amino)- 4H-benzo[e][1,2,4]thiadiazine 1,1- dioxide |
| 22-25 (S) | 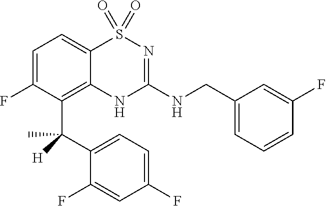 [SMILES: C[C@@H](c1ccc(F)cc1F)c1c(F)ccc2c1NC(NCc1cccc(F)c1)=NS2(=O)=O] | (S)-5-(1-(2,4- difluorophenyl)ethyl)-6-fluoro-3- ((3-fluorobenzyl)amino)-4H- benzo[e][1,2,4]thiadiazine 1,1- dioxide |
| 22-25 (R) | 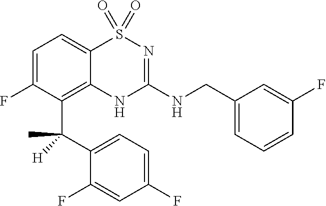 [SMILES: C[C@H](c1ccc(F)cc1F)c1c(F)ccc2c1NC(NCc1cccc(F)c1)=NS2(=O)=O] | (R)-5-(1-(2,4- difluorophenyl)ethyl)-6-fluoro-3- ((3-fluorobenzyl)amino)-4H- benzo[e][1,2,4]thiadiazine 1,1- dioxide |
| 22-26 | 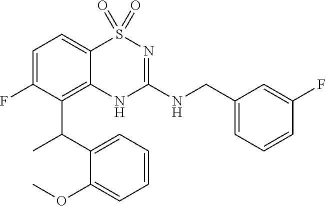 [SMILES: COc1ccccc1C(C)c1c(F)ccc2c1NC(NCc1cccc(F)c1)=NS2(=O)=O] | 6-fluoro-3-((3- fluorobenzyl)amino)-5-(1-(2- methoxyphenyl)ethyl)-4H- benzo[e][1,2,4]thiadiazine 1,1- dioxide |
| 22-26 (S) | 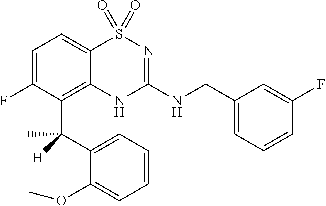 [SMILES: COc1ccccc1[C@H](C)c1c(F)ccc2c1NC(NCc1cccc(F)c1)=NS2(=O)=O] | (S)- 6-fluoro-3-((3- fluorobenzyl)amino)-5-(1-(2- methoxyphenyl)ethyl)-4H- benzo[e][1,2,4]thiadiazine 1,1- dioxide |
| 22-26 (R) | 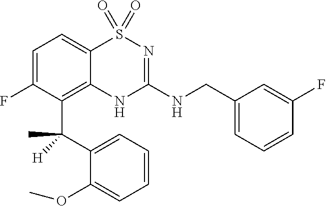 [SMILES: COc1ccccc1[C@@H](C)c1c(F)ccc2c1NC(NCc1cccc(F)c1)=NS2(=O)=O] | (R)-6-fluoro-3-((3- fluorobenzyl)amino)-5-(1-(2- methoxyphenyl)ethyl)-4H- benzo[e][1,2,4]thiadiazine 1,1- dioxide |
| 22-27 | 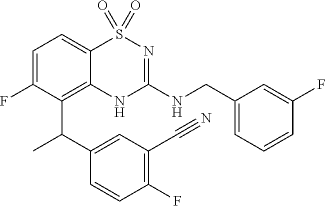 [SMILES: CC(c1ccc(F)c(C#N)c1)c1c(F)ccc2c1NC(NCc1cccc(F)c1)=NS2(=O)=O] | 2-fluoro-5-(1-(6-fluoro-3-((3- fluorobenzyl)amino)-1,1-dioxido- 4H-benzo[e][1,2,4]thiadiazin-5- yl)ethyl)benzonitrile |
| 22-27 (S) | 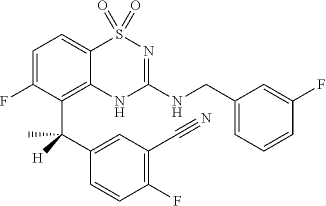 [SMILES: C[C@@H](c1ccc(F)c(C#N)c1)c1c(F)ccc2c1NC(NCc1cccc(F)c1)=NS2(=O)=O] | (S)-2-fluoro-5-(1-(6-fluoro-3-((3- fluorobenzyl)amino)-1,1-dioxido- 4H-benzo[e][1,2,4]thiadiazin-5- yl)ethyl)benzonitrile |
| 22-27 (R) | 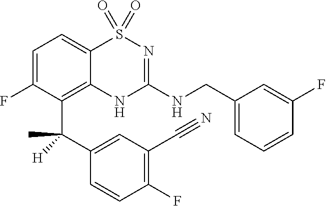 [SMILES: C[C@H](c1ccc(F)c(C#N)c1)c1c(F)ccc2c1NC(NCc1cccc(F)c1)=NS2(=O)=O] | (R)-2-fluoro-5-(1-(6-fluoro-3-((3- fluorobenzyl)amino)-1,1-dioxido- 4H-benzo[e][1,2,4]thiadiazin-5- yl)ethyl)benzonitrile |
| 22-28 | 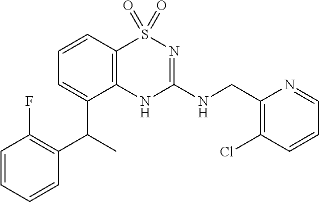 [SMILES: CC(c1ccccc1F)c1cccc2c1NC(NCc1ncccc1Cl)=NS2(=O)=O] | 5-(1-(2-fluorophenyl)ethyl)-3-(((3- chloropyridin-2-yl)methyl)amino)- 4H-benzo[e][1,2,4]thiadiazine 1,1- dioxide |
| 22-28 (S) | 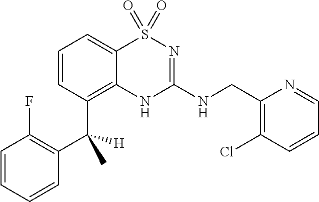 [SMILES: C[C@@H](c1ccccc1F)c1cccc2c1NC(NCc1ncccc1Cl)=NS2(=O)=O] | (S)-5-(1-(2-fluorophenyl)ethyl)-3- (((3-chloropyridin-2- yl)methyl)amino)-4H- benzo[e][1,2,4]thiadiazine 1,1- dioxide |
| 22-28 (R) | 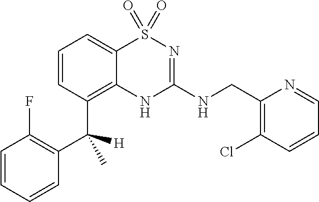 [SMILES: C[C@H](c1ccccc1F)c1cccc2c1NC(NCc1ncccc1Cl)=NS2(=O)=O] | (R)-5-(1-(2-fluorophenyl)ethyl)-3- (((3-chloropyridin-2- yl)methyl)amino)-4H- benzo[e][1,2,4]thiadiazine 1,1- dioxide |
| 22-29 | 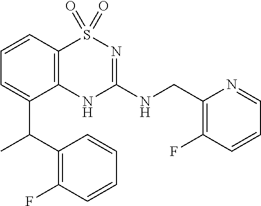 [SMILES: CC(c1ccccc1F)c1cccc2c1NC(NCc1ncccc1F)=NS2(=O)=O] | 5-(1-(2-fluorophenyl)ethyl)-3-(((3- fluoropyridin-2-yl)methyl)amino)- 4H-benzo[e][1,2,4]thiadiazine 1,1- dioxide |
| 22-29 (S) | 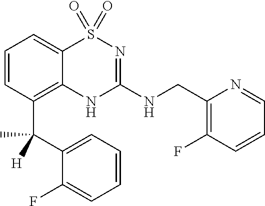 [SMILES: C[C@@H](c1ccccc1F)c1cccc2c1NC(NCc1ncccc1F)=NS2(=O)=O] | (S)-5-(1-(2-fluorophenyl)ethyl)-3- (((3-fluoropyridin-2- yl)methyl)amino)-4H- benzo[e][1,2,4]thiadiazine 1,1- dioxide |
| 22-29 (R) | 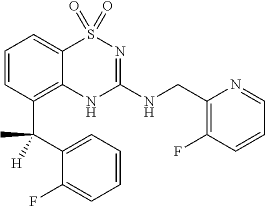 [SMILES: C[C@H](c1ccccc1F)c1cccc2c1NC(NCc1ncccc1F)=NS2(=O)=O] | (R)-5-(1-(2-fluorophenyl)ethyl)-3- (((3-fluoropyridin-2- yl)methyl)amino)-4H- benzo[e][1,2,4]thiadiazine 1,1- dioxide |
| 23-1 (racemic) | 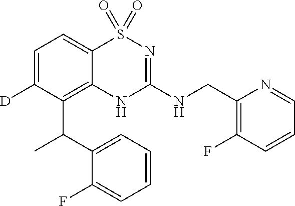 [SMILES: [2H]c1ccc2c(c1C(C)c1ccccc1F)NC(NCc1ncccc1F)=NS2(=O)=O] | 5-(1-(2-fluorophenyl)ethyl)-3-(((3- fluoropyridin-2-yl)methyl)amino)- 4H-benzo[e][1,2,4]thiadiazine 1,1- dioxide-6-d |
| 23-1 | 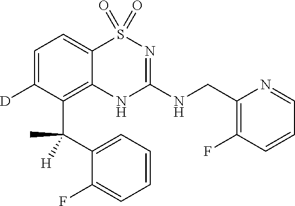 [SMILES: [2H]c1ccc2c(c1[C@H](C)c1ccccc1F)NC(NCc1ncccc1F)=NS2(=O)=O] | (S)-5-(1-(2-fluorophenyl)ethyl)-3- (((3-fluoropyridin-2- yl)methyl)amino)-4H- benzo[e][1,2,4]thiadiazine 1,1- dioxide-6-d |
| 23-1 (R) | 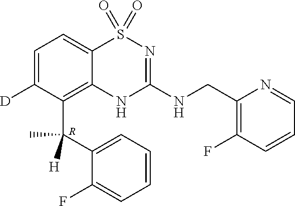 | (R)-5-(1-(2-fluorophenyl)ethyl)-3- (((3-fluoropyridin-2- yl)methyl)amino)-4H- benzo[e][1,2,4]thiadiazine 1,1- dioxide-6-d |
| 24-1 (racemic) | 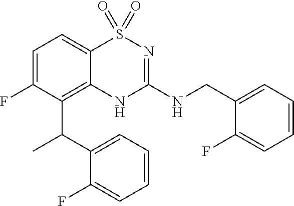 [SMILES: CC(c1ccccc1F)c1c(F)ccc2c1NC(NCc1ccccc1F)=NS2(=O)=O] | 6-fluoro-3-((2- fluorobenzyl)amino)-5-(1-(2- fluorophenyl)ethyl)-4H- benzo[e][1,2,4]thiadiazine 1,1- dioxide |
| 24-1 | 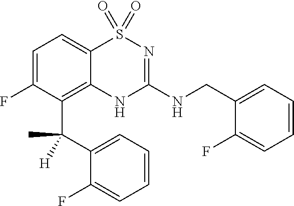 [SMILES: C[C@H](c1ccccc1F)c1c(F)ccc2c1NC(NCc1ccccc1F)=NS2(=O)=O] | (R)-6-fluoro-3-((2- fluorobenzyl)amino)-5-(1-(2- fluorophenyl)ethyl)-4H- benzo[e][1,2,4]thiadiazine 1,1- dioxide |
| 24-1 (S) | 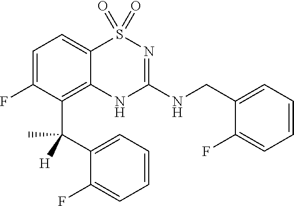 [SMILES: C[C@@H](c1ccccc1F)c1c(F)ccc2c1NC(NCc1ccccc1F)=NS2(=O)=O] | (S)-6-fluoro-3-((2- fluorobenzyl)amino)-5-(1-(2- fluorophenyl)ethyl)-4H- benzo[e][1,2,4]thiadiazine 1,1- dioxide |
| 24-2 (racemic) | 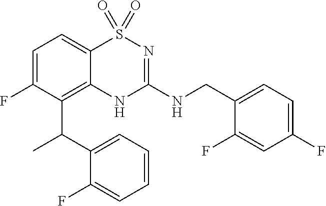 [SMILES: CC(c1ccccc1F)c1c(F)ccc2c1NC(NCc1ccc(F)cc1F)=NS2(=O)=O] | 3-((2,4-difluorobenzyl)amino)-6- fluoro-5-(1-(2-fluorophenyl)ethyl)- 4H-benzo[e][1,2,4]thiadiazine 1,1- dioxide |
| 24-2 | 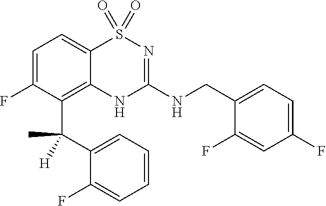 [SMILES: C[C@H](c1ccccc1F)c1c(F)ccc2c1NC(NCc1ccc(F)cc1F)=NS2(=O)=O] | (R)-3-((2,4-difluorobenzyl)amino)- 6-fluoro-5-(1-(2- fluorophenyl)ethyl)-4H- benzo[e][1,2,4]thiadiazine 1,1- dioxide |
| 24-2 (S) | 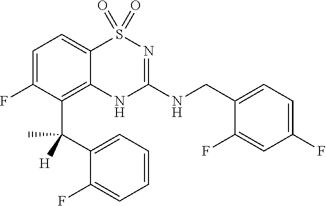 [SMILES: C[C@@H](c1ccccc1F)c1c(F)ccc2c1NC(NCc1ccc(F)cc1F)=NS2(=O)=O] | (S)-3-((2,4-difluorobenzyl)amino)- 6-fluoro-5-(1-(2- fluorophenyl)ethyl)-4H- benzo[e][1,2,4]thiadiazine 1,1- dioxide |
| 24-3 (racemic) | 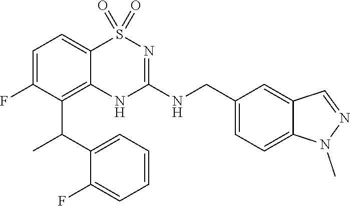 [SMILES: CC(c1ccccc1F)c1c(F)ccc2c1NC(NCc1ccc3c(cnn3C)c1)=NS2(=O)=O] | 6-fluoro-5-(1-(2- fluorophenyl)ethyl)-3-(((1-methyl- 1H-indazol-5-yl)methyl)amino)- 4H-benzo[e][1,2,4]thiadiazine 1,1- dioxide |
| 24-3 | 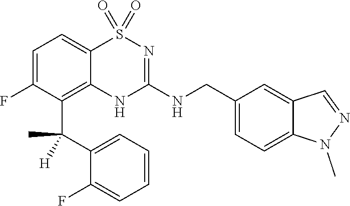 [SMILES: C[C@H](c1ccccc1F)c1c(F)ccc2c1NC(NCc1ccc3c(cnn3C)c1)=NS2(=O)=O] | (R)-6-fluoro-5-(1-(2- fluorophenyl)ethyl)-3-(((1-methyl- 1H-indazol-5-yl)methyl)amino)- 4H-benzo[e][1,2,4]thiadiazine 1,1- dioxide |
| 24-3 (S) | 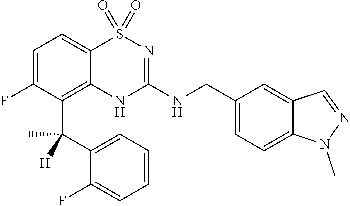 [SMILES: C[C@@H](c1ccccc1F)c1c(F)ccc2c1NC(NCc1ccc3c(cnn3C)c1)=NS2(=O)=O] | (S)-6-fluoro-5-(1-(2- fluorophenyl)ethyl)-3-(((1-methyl- 1H-indazol-5-yl)methyl)amino)- 4H-benzo[e][1,2,4]thiadiazine 1,1- dioxide |
| 24-4 (racemic) | 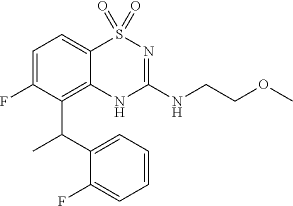 [SMILES: COCCNC1=NS(=O)(=O)c2ccc(F)c(C(C)c3ccccc3F)c2N1] | 6-fluoro-5-(1-(2- fluorophenyl)ethyl)-3-((2- methoxyethyl)amino-4H- benzo[e][1,2,4]thiadiazine 1,1- dioxide |
| 24-4 | 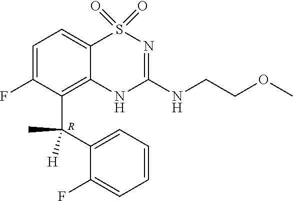 | (R)-6-fluoro-5-(1-(2- luorophenyl)ethyl)-3-((2- methoxyethyl)amino-4H- benzo[e][1,2,4]thiadiazine 1,1- dioxide |
| 24-4 (S) | 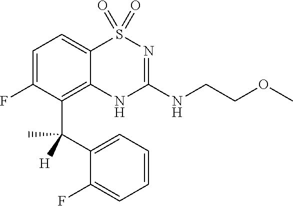 [SMILES: COCCNC1=NS(=O)(=O)c2ccc(F)c([C@@H](C)c3ccccc3F)c2N1] | (S)-6-fluoro-5-(1-(2- fluorophenyl)ethyl)-3-((2- methoxyethyl)amino-4H- benzo[e][1,2,4]thiadiazine 1,1- dioxide |
| 24-5 (racemic) | 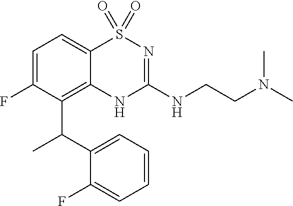 [SMILES: CC(c1ccccc1F)c1c(F)ccc2c1NC(NCCN(C)C)=NS2(=O)=O] | 6-fluoro-5-(1-(2- fluorophenyl)ethyl-3-(((2- dimethylamino)ethyl)amino)-4H- benzo[e][1,2,4]thiadiazine 1,1- dioxide |
| 24-5 | 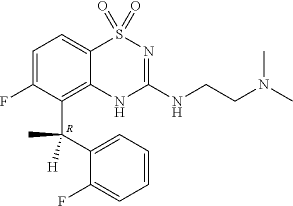 | (R)-6-fluoro-5-(1-(2- fluorophenyl)ethyl-3-(((2- dimethylamino)ethyl)amino)-4H- benzo[e][1,2,4]thiadiazine 1,1- dioxide |
| 24-5 (S) | 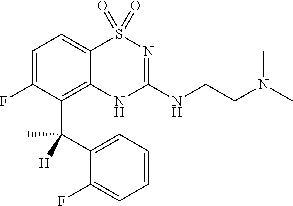 [SMILES: C[C@@H](c1ccccc1F)c1c(F)ccc2c1NC(NCCN(C)C)=NS2(=O)=O] | (S)-6-fluoro-5-(1-(2- fluorophenyl)ethyl-3-(((2- dimethylamino)ethyl)amino)-4H- benzo[e][1,2,4]thiadiazine 1,1- dioxide |
| 24-6 (racemic) | 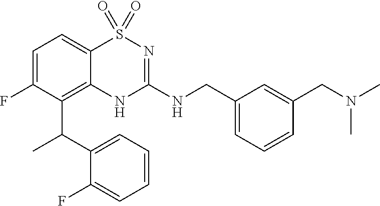 [SMILES: CC(c1ccccc1F)c1c(F)ccc2c1NC(NCc1cccc(CN(C)C)c1)=NS2(=O)=O] | 3-((3- ((dimethylamino)methyl)benzyl) amino)-6-fluoro-5-(1-(2- fluorophenyl)ethyl)-4H- benzo[e][1,2,4]thiadiazine 1,1- dioxide |
| 24-6 | 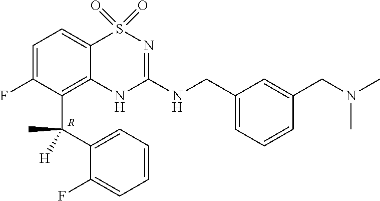 | (R)-3-((3- ((dimethylamino)methyl)benzyl) amino)-6-fluoro-5-(1-(2- fluorophenyl)ethyl)-4H- benzo[e][1,2,4]thiadiazine 1,1- dioxide |
| 24-6 (S) | 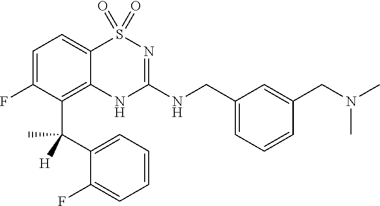 [SMILES: C[C@@H](c1ccccc1F)c1c(F)ccc2c1NC(NCc1cccc(CN(C)C)c1)=NS2(=O)=O] | (S)-3-((3- ((dimethylamino)methyl)benzyl) amino)-6-fluoro-5-(1-(2- fluorophenyl)ethyl)-4H- benzo[e][1,2,4]thiadiazine 1,1- dioxide |
| 24-7 (racemic) | 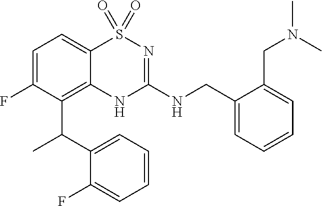 [SMILES: CC(c1ccccc1F)c1c(F)ccc2c1NC(NCc1ccccc1CN(C)C)=NS2(=O)=O] | 3-((2- ((dimethylamino)methyl)benzyl) amino)-6-fluoro-5-(1-(2- fluorophenyl)ethyl)-4H- benzo[e][1,2,4]thiadiazine 1,1- dioxide |
| 24-7 | 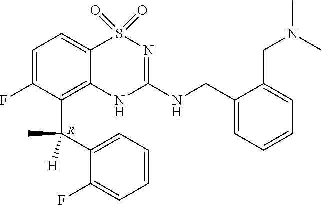 | (R)-3-((2- ((dimethylamino)methyl)benzyl) amino)-6-fluoro-5-(1-(2- fluorophenyl)ethyl)-4H- benzo[e][1,2,4]thiadiazine 1,1- dioxide |
| 24-7 (S) | 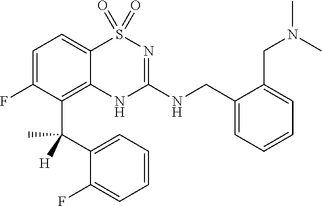 [SMILES: C[C@@H](c1ccccc1F)c1c(F)ccc2c1NC(NCc1ccccc1CN(C)C)=NS2(=O)=O] | (S)-3-((2- ((dimethylamino)methyl)benzyl) amino)-6-fluoro-5-(1-(2- fluorophenyl)ethyl)-4H- benzo[e][1,2,4]thiadiazine 1,1- dioxide |
| 24-8 (racemic) | 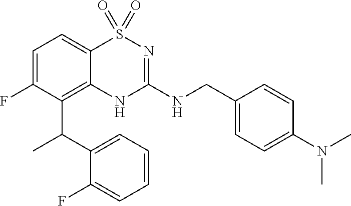 [SMILES: CC(c1ccccc1F)c1c(F)ccc2c1NC(NCc1ccc(N(C)C)cc1)=NS2(=O)=O] | 3-((4- (dimethylamino)benzyl)amino)-6- fluoro-5-(1-(2-fluorophenyl)ethyl)- 4H-benzo[e][1,2,4]thiadiazine 1,1- dioxide |
| 24-8 | 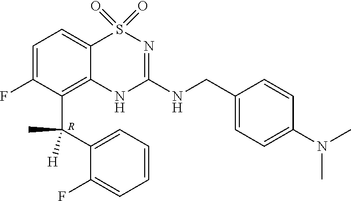 | (R)-3-((4- (dimethylamino)benzyl)amino)-6- fluoro-5-(1-(2-fluorophenyl)ethyl)- 4H-benzo[e][1,2,4]thiadiazine 1,1- dioxide |
| 24-8 (S) | 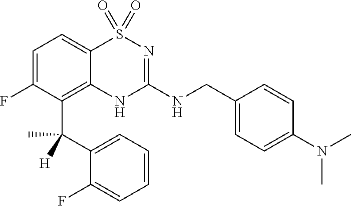 [SMILES: C[C@@H](c1ccccc1F)c1c(F)ccc2c1NC(NCc1ccc(N(C)C)cc1)=NS2(=O)=O] | (S)-3-((4- (dimethylamino)benzyl)amino)-6- fluoro-5-(1-(2-fluorophenyl)ethyl)- 4H-benzo[e][1,2,4]thiadiazine 1,1- dioxide |

“Isomer” is used herein to encompass all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized to be substantially free of their enantiomeric or diastereomeric partners, and these are all within the scope of certain embodiments of the disclosure. However, when a particular stereochemistry or isomeric form is indicated herein, the particular form is meant to indicate an assumed stereochemistry or isomeric form unless it is specifically indicated that the particular stereochemistry or isomeric form has been definitively determined. The isomers resulting from the presence of a chiral center comprise a pair of non-superimposable isomers that are called “enantiomers”. Single enantiomers of a pure compound are optically active (i.e., they can rotate the plane of plane polarized light and designated R or S). The term also encompasses isomers arising from substitution patterns across double bonds, in particular (E)- and (Z)-isomers, or cis- and trans-isomers. E-Z configuration describes the absolute stereochemistry across double bonds having two, three or four substituents. Following the Cahn-Ingold-Prelog priority rules (CIP rules), each substituent on a double bond is assigned a priority, and the positions of the higher of the two substituents on each carbon determined. If the two groups of higher priority are on the same side of the double bond (cis to each other), the bond is assigned Z (“zusammen”, German for “together”). If the two groups of higher priority are on opposite sides of the double bond (trans to each other), the bond is assigned E (“entgegen”, German for “opposite”). Each isomer may be isolated separately or exist as mixtures. The mixtures may be predominantly one isomer, e.g. 99.9%, or 99% or 90%, predominantly the other isomer, enriched in one or the other of the isomer (e.g. an 80/20 mixture, or a 40/60 mixture), or be approximately equal mixtures.

“Isolated optical isomer” means a compound which has been substantially purified from the corresponding optical isomer(s) of the same formula. For example, the isolated isomer may be at least about 80%, at least 80% or at least 85% pure. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out).

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of Formula (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has several isotopes, fluorine 19 is longest-lived. Thus, an isotope of a compound having the structure of Formula (I) includes, but not limited to, compounds of Formula (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

In one embodiment is provided an isotope of a compound of Table 1 or of any one of Formulas (I), (II), (III), (III-A), or (III-B). In one embodiment, the isotope comprises at least one deuterium atom. In another embodiment, an isotope is provided wherein L² is —CD₂-. In another embodiment, an isotope is provided wherein at least one R⁴, R⁵, or R⁶ is D.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers to an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the disclosure include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, Phydroxybutyric, salicylic, -galactaric, and galacturonic acid.

Although pharmaceutically unacceptable salts are not generally useful as medicaments, such salts may be useful, for example as intermediates in the Synthesis of the compounds described herein, for example in their purification by recrystallization.

In some embodiments, the compounds are pharmaceutically acceptable salts. In some embodiments, the compounds are isomers. In some embodiments, the compounds are racemates. In some embodiments, the compounds are solvates. In some embodiments, the compounds are hydrates. In some embodiments, the compounds are isotopes.

### Pharmaceutical Compositions

In certain embodiment, also disclosed herein are pharmaceutical compositions comprising a compound as described herein, or a pharmaceutically acceptable salt, solvate, hydrate, isomer, tautomer, racemate or isotope thereof. In some embodiments, the pharmaceutical compositions further comprise a pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, homolog or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21st Ed., Gennaro, Ed., Lippencott Williams & Wilkins (2005) and in The United States Pharmacopeia: The National Formulary (USP 36 NF31), published in 2013.

In other embodiments, there are provided methods of making a composition of a compound described herein including formulating a compound of the disclosure with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation.

As used herein, the term “pharmaceutically acceptable carrier” refers to any ingredient other than the disclosed compounds, or a pharmaceutically acceptable isomer, racemate, hydrate, solvate, homolog or salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the disclosure to the appropriate or desired site of action, such as oral, nasal, pulmonary, buccal, subdermal, intradermal, transdermal, or parenteral, e.g., rectal, depot, subcutaneous, intravenous, intraurethral, intramuscular, intranasal, ophthalmic solution, or an ointment, the oral route being preferred.

Dosage forms can be administered once a day, or more than once a day, such as twice or thrice daily. Alternatively, dosage forms can be administered less frequently than daily, such as every other day, or weekly, if found to be advisable by a prescribing physician. Dosing regimens include, for example, dose titration to the extent necessary or useful for the indication to be treated, thus allowing the patient's body to adapt to the treatment and/or to minimize or avoid unwanted side effects associated with the treatment. Other dosage forms include delayed or controlled-release forms. Suitable dosage regimens and/or forms include those set out, for example, in the latest edition of the Physicians' Desk Reference, incorporated herein by reference.

Modulating MRGPRX2 Activity and Treating Diseases Associated with MRGPRX2

In certain embodiments, described herein, are methods for modulating the activity of at least one Mas-related G protein coupled receptor (MRGPR) comprising contacting the MRGPR with a compound as described herein, or a pharmaceutically acceptable salt, solvate, hydrate, isomer, tautomer, racemate, isotope, or composition thereof. In some embodiments, the MRGPR is MRGPRX2. In certain embodiments, methods of treating a subject having a disease or disorder associated with the activity of MRGPRX2 are disclosed, the method comprising administering to a subject in need thereof a pharmaceutically effective amount of a compound as described herein, or a pharmaceutically acceptable salt, solvate, hydrate, isomer, tautomer, racemate, isotope, or composition thereof.

“Modulating” MRGPRX2 means that the compound interacts with MRGPRX2 or MRGPRX2 orthologs in a manner such that it functions as an inverse agonist to the receptor, and/or as a competitive antagonist to the receptor. In one embodiment, such modulation is partially or fully selective against other MRGPRs, such as MRGPR X1, X3, X4 and/or D.

“MRGPR” refers to one or more of the Mas-related G protein coupled receptors, which are a group of orphan receptors with limited expression in very specialized tissues (e.g., in mast cells and dorsal root ganglia) and barrier tissues. There are eight related receptors in this class expressed in humans. Of these, four members (MRGPRD, E, F and G) have readily identifiable orthologs in mammals. The other four receptors (MRGPR X1, X2, X3 and X4) have no single ortholog, based on homology, in non-human species. Among rodent receptors, mouse mrgprb2 and rat mrgprb3 correspond functionally to human MRGPRX2 on mast cells.

“MRGPRX2,” also referred to as “MRGX2,” or “MGRG3,” refers to a member of the MRGPR family that is expressed on mast cells and capable of mediating IgE independent activation (e.g., mast cell degranulation) in response to ligand binding. An exemplary human MRGPRX2 amino acid sequence is set forth in Uniprot Q96LB1.

As used herein, the term “administering” or “administration” refers to providing a compound, a pharmaceutical composition comprising the same, to a subject by any acceptable means or route, including (for example) by oral, parenteral (e.g., intravenous), or topical administration.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed. The terms cover the treatment of a disease-state in a mammal, particularly in a human, and include: (a) preventing the disease-state from occurring in a mammal, in particular, when such mammal is predisposed to the disease state but has not yet been diagnosed as having it; (b) inhibiting the disease-state, i.e., arresting its development; and/or (c) relieving the disease-state, i.e., causing regression of the disease state.

As used herein, the term “subject” refers to an animal (e.g., a mammal, such as a human, dog or horse), and thus veterinary use is an application specifically contemplated herein. A subject to be treated according to the methods described herein may be one who has been diagnosed with a MRGPRX2 dependent condition or MRGPRX2 ortholog dependent condition, such as a pseudo-allergic reaction, an itch associated condition, a pain associated condition, a cancer associated condition, an inflammatory or autoimmune disorder. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition. The term “patient” may be used interchangeably with the term “subject.” A subject may refer to an adult or pediatric subject.

The Federal Food, Drug, and Cosmetic Act defines “pediatric” as a subject aged 21 or younger at the time of their diagnosis or treatment. Pediatric subpopulations are further characterized as: (i) neonates—from birth through the first 28 days of life; (ii) infants—from 29 days to less than 2 years; (iii) children—2 years to less than 12 years; and (iv) adolescents—aged 12 through 21. Despite the definition, depending on the susceptible patient population and clinical trial evaluation, an approved regulatory label may include phrasing that specifically modifies the range of a pediatric population, such as, for example, pediatric patients up to 22 years of age.

As used herein, the term “effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

As used herein, the term “therapeutically effective amount” or “pharmaceutically effective amount” is intended to include an amount of a compound of the present invention alone or an amount of a compound of the present invention in combination with other active ingredients effective to act as a modulator of MRGPRX2 or effective to treat or prevent a disease or disorder associated with MRGPRX2.

As used herein, the term “contacting” refers to the bringing together of indicated moieties in an in vitro system or an in vivo system. For example, “contacting” the MRGPRX2 ortholog with a compound of Formula (I) includes the administration of a compound of the present invention to an individual or patient, such as a human, having MRGPRX2, as well as, for example, introducing a compound of Formula (I) into a sample containing a cellular or purified preparation containing MRGPRX2.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the phrase “MRGPRX2 dependent condition” means a condition where the activation, over sensitization, or desensitization of MRGPRX2 or its orthologs by a natural or synthetic ligand initiates, mediates, sustains, or augments a pathological condition. For example, it is known that some cationic peptidergic drugs cause pseudo-allergic reactions in patients where MRGPRX2 is sensitive to (or activated by) secretagogues, cationic peptidergic drugs, including Icatibant, Leuprolide, or Ganirelix, neutral and anionic peptidergic drugs (e.g., Exenatide, Glucagon, Liraglutide, Enfuvirtide, Colistimethate), non-steroidal agonist (atracurium mivacurium), non-steroidal antagonist drugs, neuropeptides, and antimicrobial peptides. Moreover, overexpression of MRGPRX2 and/or overactivity MRGPRX2 may also render mast cells more susceptible to activation by endogenous and/or exogenous ligands. Without being limited by theory, it is to be understood that by modulating MRGPRX2, pseudo-allergic reactions, itch, pain, inflammatory or autoimmune disorders can be eased.

In some embodiments, the MRGPRX2 dependent condition is a condition that is caused by IgE independent activation of MRGPRX2 or its orthologs. IgE independent activation of MRGPRX2 is capable of inducing mast cell degranulation. For example, IgE independent mast cell activation is associated with some cases of chronic urticaria and other mast cell mediated conditions, which are not responsive to current anti-IgE or antihistamine therapies. Thus, the compounds of the present disclosure may be used for treating an MRGPRX2 dependent condition caused by IgE independent activation of MRGPRX2 and that would benefit from modulating MRGPRX2.

In some embodiments, the MRGPRX2 dependent condition is an itch associated condition, a pain associated condition, a cancer associated condition, a pseudo-allergic reaction, or an autoimmune or inflammatory disorder in humans or other mammals.

As used herein the phrase “pseudo-allergic reaction” refers to an IgE-independent allergic reaction, characterized by histamine release, inflammation, airway contraction, or any combination thereof. A pseudo-allergic reaction may be an anaphylactic reaction. A pseudo-allergic reaction may be caused by a range of cationic substances, collectively called basic secretagogues, including inflammatory peptides and drugs associated with allergic-type reactions. Thus, in one embodiment, the method of present invention is provided to treat a pseudo-allergic reaction, such as pseudo-allergic reactions caused by secretagogues, cationic peptidergic drugs, anionic peptidergic drugs, neutral peptidergic drugs, non-steroidal antagonist drugs, neuropeptides, and antimicrobial peptides. In one embodiment, the pseudo-allergic reaction is caused by MCD peptide, Substance P, VIP, PACAP, dynorphin, somatostatin, Compound 48/80, Cortistatin-14, Mastoparan, Melettin, Cathelicidin Peptides, Ciprofloxacin, Vancomycin, Leuprolide, Goserelin, Histrelin, Triptorelin, Cetrorelix, Ganirelix, Degarelix, Octreotide, Lanreotide, Pasireotide, Sermorelin, Tesamorelin, Icatibant, Glatiramer Acetate, Teriparatide, Pramlintide, Bleomycin, Exenatide, Glucagon, Liraglutide, Enfuvirtide, Colistimethate, Succinylcholine, Tubocurarine, Atracurium, Mivacurium, And Rocuronium.

As used herein, the phrase “itch associated condition” means pruritus (including acute and chronic pruritus) associated with any condition. The itch sensation can originate, e.g., from the peripheral nervous system (e.g., dermal or neuropathic itch) or from the central nervous system (e.g., neuropathic, neurogenic or psychogenic itch). Thus, in one embodiment, the method of present invention is provided to treat an itch associated condition, such as acne, allergic blepharitis, anaphylaxis, anaphylactoid drug reactions, anaphylactic shock, anemia, atopic dermatitis, bullous pemphigoid, burn healing, candidiasis, chicken pox, cholestatic pruritis, chronic itch, chronic urticaria, contact dermatitis, cutaneous amyloidosis, dermatitis herpetiformis, diabetes, drug allergy, dry skin, dyshidrotic dermatitis, ectopic eczema, end-stage renal failure, eosinophilic fasciitis, epidermolysis bullosa, erythrasma, food allergy, folliculitis, fungal skin infection, hemodialysis, hemorrhoids, herpes, HIV infection, Hodgkin's disease, hyperthyroidism, iodinated contrast dye allergy, iron deficiency anemia, kidney disease, leukemia, porphyria, lymphoma, mast cell activation syndrome, malignancy, mastocytosis, multiple myeloma, neurodermatitis, ocular itch, onchocerciasis, Paget's disease, pediculosis, polycythemia rubra vera, prurigo nodularis, lichen planus, lichen sclerosis, pruritus ani, pseudo-allergic reactions, pseudorabies, psoriasis, rectal prolapse, rosacea, sarcoidosis granulomas, scabies, schistosomiasis, scleroderma, severe stress, Stasia dermatitis, swimmer's itch, thyroid disease, tinea cruris, uremic pruritus, urticaria and wound healing.

As used herein, the phrase “pain associated condition” means any pain due to a medical condition. Thus, in one embodiment, the method of present invention is provided to treat a pain associated condition, such as acute pain, advanced prostate cancer, AIDS-related pain, ankylosing spondylitis, arachnoiditis, arthritis, arthrofibrosis, ataxic cerebral palsy, autoimmune atrophic gastritis, avascular necrosis, back pain, Behcet's Disease (Syndrome), bladder pain, burning mouth syndrome, bursitis, cancer pain, carpal tunnel, cauda equina syndrome, central pain syndrome, cerebral palsy, cervical stenosis, Charcot-Marie-Tooth (CMT) disease, chronic fatigue syndrome (CFS), chronic functional abdominal pain (CFAP), chronic pain, chronic pancreatitis, chronic pelvic pain syndrome, collapsed lung (pneumothorax), complex regional pain syndrome (RSD), corneal neuropathic pain, Crohn's disease, degenerative disc disease, dental pain, Dercum's disease, dermatomyositis, diabetic peripheral neuropathy (DPN), dystonia, Ehlers-Danlos syndrome (EDS), endometriosis, eosinophilia-myalgia syndrome (EMS), erythromelalgia, fibromyalgia, gout, headaches, herniated disc, hydrocephalus, intercostal neuralgia, interstitial cystitis, irritable bowel syndrome (IBS), juvenile dermatosis and dermatomyositis, knee injury, leg pain, loin pain-haematuria syndrome, lupus, Lyme disease, medullary sponge kidney (MSK), meralgia paresthetica, mesothelioma, migraine, musculoskeletal pain, myofascial pain, myositis, neck pain, neuropathic pain, occipital neuralgia, osteoarthritis, Paget's disease, Parsonage Turner syndrome, pelvic pain, periodontitis pain, peripheral neuropathy, phantom limb pain, pinched nerve, polycystic kidney disease, polymyalgia rheumatica, polymyositis, porphyria, post herniorrhaphy pain syndrome, post mastectomy, postoperative pain, pain syndrome, post stroke pain, post thoracotomy pain syndrome, postherpetic neuralgia (shingles), post-polio syndrome, primary lateral sclerosis, psoriatic arthritis, pudendal neuralgia, radiculopathy, Raynaud's Disease, rheumatoid arthritis (RA), sacroiliac joint dysfunction, sarcoidosis, Scheuermann's Kyphosis disease, sciatica, scoliosis, shingles (herpes zoster), Sjogren's syndrome, spasmodic torticollis, sphincter of Oddi dysfunction, spinal cerebellum ataxia (SCA Ataxia), spinal cord injury, spinal stenosis, syringomyelia, Tarlov cysts, transverse myelitis, trigeminal neuralgia, ulcerative colitis, vascular pain and vulvodynia.

As used herein, the term “autoimmune disorder”, or “inflammatory disorder” means a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues. Thus, in one embodiment, the method of present invention is provided to treat an autoimmune disorder, such as chronic inflammation, mast cell activation syndrome, Multiple Sclerosis, Steven Johnson's Syndrome, Toxic Epidermal Necrolysis, acne vulgaris, appendicitis, bladder infection, bursitis, cutaneous lupus, colitis, cystitis, dermatitis, phlebitis, reflex sympathetic dystrophy/complex regional pain syndrome (rsd/crps), rhinitis, rosacea, tendonitis, tonsillitis, sinusitis, psoriasis, graft-versus-host disease, reactive airway disorder, asthma, airway infection, allergic rhinitis, autoinflammatory disease, celiac disease, chronic prostatitis, Crohn's Disease, diverticulitis, endometrial pain, epithelial intestinal disorder, glomerulonephritis, hidradenitis suppurativa, hypersensitivities, interstitial cystitis, intestinal disorder, inflammatory bowel disease, irritable bowel syndrome, lupus erythematous, meningitis, otitis, pelvic inflammatory disease, reperfusion injury, rheumatic fever, rheumatoid arthritis, sarcoidosis, transplant rejection, psoriasis, lung inflammation, chronic obstructive pulmonary disease, permanent sputum eosinophilia, eosinophilic leukemia, eosinophilic esophagitis, eosinophilic gastritis, eosinophilic duodenitis, eosinophilic gastroenteritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, schwannomatosis, tubulointerstitial nephritis, ulcerative colitis, glomerulonephritis, diabetic nephropathy, allograft rejection, amyloidosis, renovascular ischemia, reflux nephropathy, polycystic kidney disease, liver fibrosis/cirrhosis, autoimmune liver disease, biliary atresia, acute and chronic Hepatitis B and C virus, Liver tumors and cancer, alcoholic liver disease, polycystic liver disease, liver cholangiocarcinoma, neuromyelitis optica spectrum disorder, cardiovascular disease, inflammation induced by bacterial or viral infection, inflammation associated with SARS-CoV-2 infection or its variants and Coronavirus Disease 2019 (COVID-19), acute respiratory distress syndrome, pneumonia, Long/Long-Term/Chronic COVID, Post-Acute Sequelae of COVID-19 (PASC), myalgic encephalomyelitis/chronic fatigue syndrome (ME/CFS “Brain Fog”) and vasculitis.

As used herein the phrase “cancer associated condition” means any disease arising from the proliferation of malignant cancerous cells. Thus, in one embodiment, the method of present invention is provided to treat a cancer/tumor associated condition, such as adenoid cystic carcinoma, adrenal gland tumor, amyloidosis, anal cancer, appendix cancer, astrocytoma, ataxia-telangiectasia, Beckwith Wiedemann syndrome, Birt-Hogg-Dubee syndrome, bone cancer, brain stem glioma, brain tumor, breast cancer (inflammatory, metastatic, male), cholangiocarcinoma, prostrate, basal cell, melanoma, colon, colorectal, bladder, kidney cancer, lacrimal gland cancer, laryngeal and hypopharyngeal cancer, lung cancer (non-small cell, small cell), leukemia (acute lymphoblastic, acute lymphocytic, acute myeloid, B cell prolymphocytic, chronic lymphocytic, chronic myeloid, chronic T cell lymphocytic, eosinophilic), Liver Cancer, Li-Fraumeni syndrome, lymphoma (Hodgkin and non-Hodgkin), lynch syndrome, mastocytosis, medulloblastoma, meningioma, mesothelioma, multiple endocrine neoplasia, multiple myeloma, MUTYH-associated polyposis, myelodysplastic syndrome, nasal cavity and paranasal sinus cancer, neuroblastoma, neuroendocrine tumors, neurofibromatosis, penile cancer, parathyroid cancer, ovarian fallopian tube and peritoneal cancer, osteosarcoma, pituitary gland tumor, pleuropulmonary blastoma, oral and oropharyngeal, thyroid, uterine, pancreatic, carney complex, brain and spinal cord cancer, cervical cancer, Cowden syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor, germ cell tumor, gestational trophoblastic disease, head and neck cancer, hereditary breast and ovarian cancer, hereditary diffuse gastric cancer, hereditary, leiomyomatosis and renal cell cancer, hereditary pancreatitis, hereditary papillary renal carcinoma, hereditary mixed polyposis syndrome, HIV/AIDS related cancers, retinoblastoma, rhabdomyosarcoma, salivary gland cancer, Kaposi sarcoma, small bowel cancer, stomach cancer, testicular cancer, thymoma and thymic carcinoma, thyroid cancer, vaginal cancer, culver cancer, Werner syndrome and Xeroderma pigmentosum.

In another embodiment, a method of treating a subject having an itch associated condition is provided, the method comprising administering to the subject a pharmaceutically effective amount of a compound having structure (I) or a pharmaceutically acceptable salt, isomer, hydrate, solvate or isotope thereof, or a pharmaceutical composition thereof. In one embodiment, the itch associated condition is urticaria, pruritus, atopic dermatitis, dry skin, psoriasis, contact dermatitis, or eczema. In another embodiment, a method of treating a subject having an inflammation or autoimmune associated condition is provided, the method comprising administering to the subject a pharmaceutically effective amount of a compound having structure (I) or a pharmaceutically acceptable salt, isomer, hydrate, solvate or isotope thereof, or a pharmaceutical composition thereof. In one embodiment, the inflammation or autoimmune associated condition is sinusitis, asthma, rosacea, or endometriosis.

In another embodiment, a method of treating a subject having a pain associated condition is provided, the method comprising administering to the subject a pharmaceutically effective amount of a compound having structure (I) or a pharmaceutically acceptable salt, isomer, hydrate, solvate or isotope thereof, or a pharmaceutical composition thereof. In one embodiment, the pain associated condition is chronic pelvic pain syndrome, endometriosis pain, fibromyalgia, migraine or postoperative pain.

### Combination Therapy

In another embodiment, the method of treating a subject having a MRGPRX2 dependent condition (e.g., an itch associated condition, a pain associated condition, a pseudo-allergic reaction, or an inflammatory or autoimmune disorder) described herein further comprises administering to the subject a pharmaceutically effective amount of a second therapeutic agent. In one embodiment, the itch associated condition is a pseudo-allergic condition.

In one embodiment, the second therapeutic agent is an antihistamine, such as an H1 receptor antagonist or an H2 receptor antagonist. In one embodiment, the second therapeutic agent is an H1 receptor antagonist antihistamine, such as levocetirizine, loratadine, fexofenadine, cetirizine, desloratadine, olopatadine, diphenhydramine, cyproheptadine or hydroxyzine pamoate. In one embodiment, the second therapeutic agent is a H2 receptor antagonist, such as cimetidine, nizatidine, ranitidine or famotidine. In one embodiment, the second therapeutic agent is a leukotriene receptor antagonist or leukotriene synthesis inhibitor, such as Montelukast, Zafirlukast, Pranlukast, or 5-lipoxygenase inhibitor (e.g., Zileuton, hypericum perforatum). In one embodiment, the second therapeutic agent is an immunomodulatory agent such as Omalizumab or immunoglobulin therapy. In one embodiment, the second therapeutic agent is a corticosteroid, such as hydrocortisone, cortisone, betamethasone, triamcinolone, prednisone, prednisolone, or fludrocortisone. In one embodiment, the second therapeutic agent is a tricyclic antidepressant that can relieve itch such as doxepin, amitriptyline or nortriptyline. In one embodiment, the second therapeutic agent is an anti-inflammatory drug such as dapsone, sulfasalazine, hydroxychloroquine or colchicine. In one embodiment, the second therapeutic agent is an immunosuppressant such as cyclosporine, methotrexate, mycophenolic acid or tacromilus.

The second therapeutic agent may be administered simultaneously, separately, or sequentially with the compounds of the present disclosure. If administered simultaneously, the second therapeutic agent and compound of the present disclosure may be administered in separate dosage forms or in the same dosage form.

### Routes of Administration

The compounds of this invention can be administered for any of the uses described herein by any suitable means, for example, orally, such as tablets, capsules (each of which includes sustained release or timed release formulations), pills, powders, granules, elixirs, tinctures, suspensions (including nanosuspensions, micro suspensions, spray-dried dispersions), syrups, and emulsions; sublingually; buccally; parenterally, such as by subcutaneous, intravenous, intramuscular, or intratarsal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

### Kits

The present invention also includes pharmaceutical kits useful, for example, in the treatment or prevention of MRGPRX2 associated diseases or disorders, and other diseases referred to herein, which include one or more containers containing a pharmaceutical composition comprising a therapeutically effective amount of a compound of Formula (I). Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

### Compound Synthesis

Compounds having the structure of Formulas (I) can be synthesized using standard synthetic techniques known to those of skill in the art.

Modifications to the methods described herein will be apparent to one skilled in the art. To this end, the reactions, processes, and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary. Generally, suitable solvents are protic or aprotic solvents which are substantially non-reactive with the reactants, the intermediates, or products at the temperatures at which the reactions are carried out (i.e., temperatures which may range from the freezing to boiling temperatures). A given reaction may be carried out in one solvent or a mixture of more than one solvent. Depending on the particular reaction, suitable solvents for a particular work-up following the reaction may be employed.

Unless otherwise indicated, conventional methods of mass spectroscopy (MS), liquid chromatography-mass spectroscopy (LCMS), NMR, HPLC, protein chemistry, biochemistry, recombinant DNA techniques, and pharmacology are employed. Compounds are prepared using standard organic chemistry techniques such as those described in, for example, March's Advanced Organic Chemistry, 7th Edition, John Wiley and Sons, Inc (2013). Alternate reaction conditions for the synthetic transformations described herein may be employed such as variation of solvent, reaction temperature, reaction time, as well as different chemical reagents and other reaction conditions. As necessary, the use of appropriate protecting groups may be required. The incorporation and cleavage of such groups may be carried out using standard methods described in Peter G. M. Wuts and Theodora W. Green, Protecting Groups in Organic Synthesis, 4th Edition, Wiley-Interscience. (2006). All starting materials and reagents are commercially available or readily prepared.

### EXAMPLES

### General Methods

¹H NMR (400 MHz) spectra were obtained in solution of deuterochloroform (CDCl₃), deuteromethanol (CD₃OD) or dimethyl sulfoxide-D₆ (DMSO-D₆). HPLC retention times, purities and mass spectra (LCMS) were obtained using one of the following methods:

Purity Method 1: Shimadzu system equipped with a Halo C18, 5 μm, 3.0×30 mm column using H₂O with 0.0375% trifluoroacetic acid as mobile phase A, and acetonitrile with 0.01875% trifluoroacetic acid as mobile phase B. The gradient was 5-95% mobile phase B over 0.5 min, held at 95% mobile phase B for 0.3 min, returned to 5% mobile phase B for 0.205 min. The flow rate was 1.5 mL/min.

Purity Method 2: Shimadzu system equipped with a Kinetex EVO C18, 5 μm, 2.1×30 mm column using H₂O with 0.025% NH₄OH as mobile phase A, and acetonitrile as mobile phase B. The gradient was 0-60% mobile phase B over 0.8 min, held at 60% mobile phase B for 0.4 min, returned to 0% mobile phase B for 0.35 min. The flow rate was 1.5 mL/min.

Purity Method 3: Agilent 1260 Infinity II System equipped with an Agilent Poroshell 120 EC-18, 2.7 μm, 4.6×100 mm column, using H₂O with 0.1% formic acid as mobile phase A, and MeCN with 0.1% formic acid as mobile phase B. The gradient was 10-95% mobile phase B over 12 min, held at 95% for 2 min, then returned to 10% mobile phase B over 1 min. The flow rate was 1 mL/min. An ESI detector in positive mode was used.

Purity Method 4: Agilent 1260 Infinity II System equipped with an Agilent Poroshell 120 EC-18, 2.7 μm, 4.6×100 mm column, using H₂O with 0.1% formic acid as mobile phase A, and MeCN with 0.1% formic acid as mobile phase B. The gradient was 5-95% mobile phase B over 5 min, 95-20% mobile phase B over 1.5 min, then returned to 10% mobile phase B over 0.5 min. The flow rate was 1 mL/min. An ESI detector in positive mode was used.

Chiral Purity Method 1: Shimadzu system equipped with a Chiralcel OJ-3, 3 μm, 4.6×50 mm column at 35° C. column temp using MeOH (0.05% DEA) in CO₂ from 5% to 40% gradient over 3 min. The flow rate was 3 mL/min. A PDA detector was used.

Chiral Purity Method 2: Shimadzu system equipped with a Chiralcel OJ-3, 3 μm, 4.6×50 mm column at 35° C. column temp using EtOH (0.05% DEA) in CO₂ from 5% to 40% gradient over 3 min. The flow rate was 3 mL/min. A PDA detector was used.

The pyridine, dichloromethane (DCM), tetrahydrofuran (THF), and toluene used in the procedures were from Aldrich Sure-Seal bottles kept under nitrogen (N₂). All reactions were stirred magnetically, and temperatures are external reaction temperatures. Chromatographies were typically carried out using a Combiflash Rf flash purification system (Teledyne Isco) equipped with Redisep (Teledyne Isco) Rf Gold Normal-Phase silica gel (SiO₂) columns or by using a similar system.

Preparative HPLC purifications were typically performed using one of the following systems: 1) Waters System equipped with a Waters 2489 uv/vis detector, an Aquity QDA detector, a Waters xBridge Prep C18 5 μm OBD, 30×150 mm column, and eluting with various gradients of H₂O/MeCN (0.1% formic acid) at a 30 mL/min flow rate, 2) Teledyne Isco ACCQPrep® HP150 UV system equipped with a Waters xBridge Prep C18 5 μm OBD, 30×150 mm column, and eluting with various gradients of H₂O/MeCN (0.1% formic acid) at a 42.5 mL/min flow rate, or 3) column: Phenomenex Synergi C18 150×30 mm-4 m; mobile phase: [H₂O (0.225% formic acid)-MeCN]; B %: 55%-85%, 12 min) and were typically concentrated using a Genevac EZ-2.

The following additional abbreviations are used: ethyl acetate (EA), triethylamine (TEA), dimethyl sulfoxide (DMSO), silica gel (SiO₂), azobisisobutyronitrile (AIBN), diisobutylaluminium hydride (DIBAL), trifluoroacetic acid (TFA), 4-dimethylaminopyridine (DMAP), diphenylphosphoryl azide (DPPA), benzoyl peroxide (BPO), 1,1′-bis(diphenylphosphino)ferrocene (dppf), bis(pinacolato)diboron (B₂pin₂), tetrahydrofuran (THF), 1,4-diazabicyclo[2.2.2]octane bis(sulfur dioxide) adduct (DABSO), hexafluorophosphate azabenzotriazole tetramethyl uronium (HATU), hydroxybenzotriazole (HOBt), N-methyl morpholine (NMM), N-Bromosuccinimide (NBS), diisopropylethyl amine (DIPEA), diethyl azodicarboxylate (DEAD), diisopropyl azodicarboxylate (DIAD), 2-[2-(dicyclohexylphosphino)phenyl]-N-methylindole (CM-Phos), triflic acid (TfOH), 1-ethyl-3-(3-dimethylaminopropyl) carbodiimide hydrochloride (EDC), isopropanol (IPA), dimethylformamide (DMF), dimethyl acetamide (DMA), dichloromethane (DCM), 1,2-dichloroethane (DCE), acetonitrile (MeCN or ACN), 1,1′-thiocarbonyldiimidazole (TCDI), petroleum ether (PE), not determined (ND), retention time (RT), molecular weight (mw), room temperature (rt), hour (h), and not applicable (N/A).

### Example 1

### Synthesis of Representative Compound 1-15

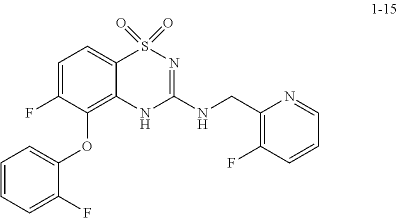 [SMILES: O=S1(=O)N=C(NCc2ncccc2F)Nc2c1ccc(F)c2Oc1ccccc1F]

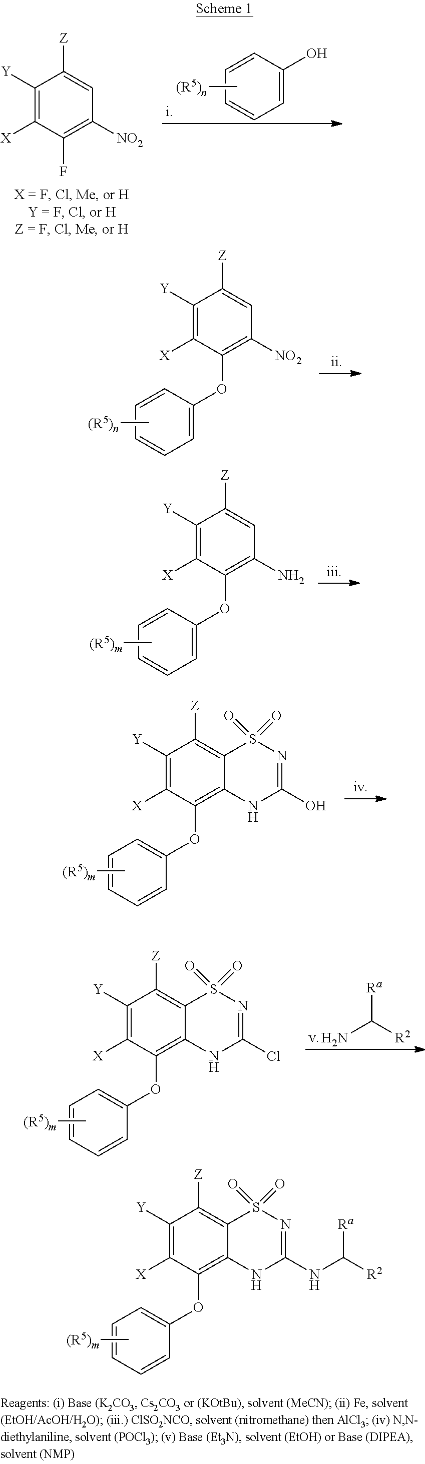

### Step 1-1. Synthesis of 1-fluoro-2-(2-fluorophenoxy)-3-nitrobenzene (INT-1A)

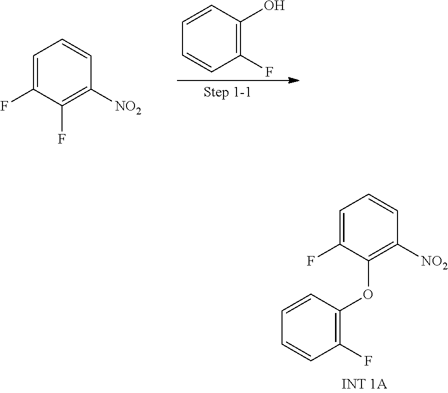 [SMILES: O=[N+]([O-])c1cccc(F)c1F ; Oc1ccccc1F ; O=[N+]([O-])c1cccc(F)c1Oc1ccccc1F]

To 2-fluorophenol (0.500 g, 1 Eq, 4.46 mmol) in MeCN (10 mL) was added Potassium carbonate (616 mg, 261 μL, 1 Eq, 4.46 mmol) and 1,2-difluoro-3-nitrobenzene (710 mg, 1 Eq, 4.46 mmol). The reaction vial was capped and heated at 80° C. overnight. After the reaction was completed, water (10 mL) was added. The aqueous layer was extracted with EtOAc (3×10 mL), and the organics were concentrated in vacuo to afford crude material. The crude material was purified by silica gel chromatography (0-50% EtOAc/hexanes) to afford 1-fluoro-2-(2-fluorophenoxy)-3-nitrobenzene INT 1A (1.04 g, 4.14 mmol, 93% yield).

### Step 1-2. Synthesis of 3-fluoro-2-(2-fluorophenoxy)aniline (INT-1B)

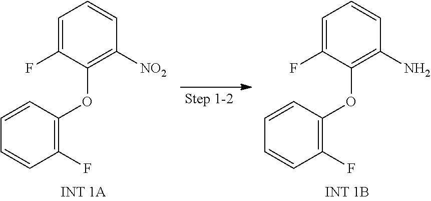 [SMILES: O=[N+]([O-])c1cccc(F)c1Oc1ccccc1F ; Nc1cccc(F)c1Oc1ccccc1F]

To a vigorously stirred solution of INT 1A (1.04 g, 1 Eq, 4.14 mmol) in EtOH (6 mL), H2O (6 mL), and AcOH (6 mL) was added iron powder (925 mg, 4 Eq, 16.6 mmol). The reaction vial was capped and heated at 100° C. overnight. The reaction mixture was filtered through Celite, rinsed with EtOAc, concentrated in vacuo, and purified by silica gel chromatography (0-100% EtOAc/hexanes) to afford 3-fluoro-2-(2-fluorophenoxy)aniline INT 1B (871 mg, 3.94 mmol, 95% yield).

### Step 1-3. Synthesis of 6-fluoro-5-(2-fluorophenoxy)-3-hydroxy-4H-benzo[e][1,2,4]thiadiazine 1,1-dioxide (INT 1C)

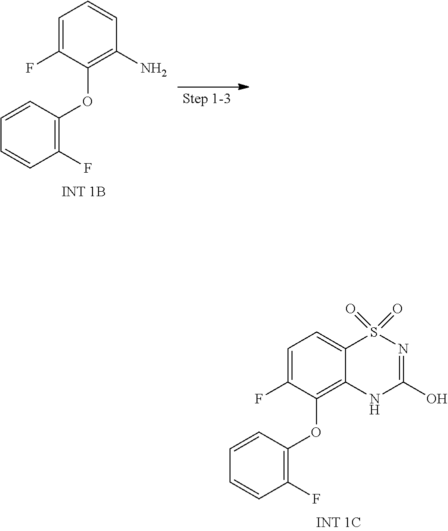 [SMILES: Nc1cccc(F)c1Oc1ccccc1F ; O=S1(=O)N=C(O)Nc2c1ccc(F)c2Oc1ccccc1F]

To a solution of sulfurisocyanatidic chloride (668 mg, 1.2 Eq, 4.72 mmol) in nitromethane (15 mL) at 0° C. was added dropwise a solution of INT 1B (870 mg, 1 Eq, 3.93 mmol) in nitromethane (15 mL). The reaction mixture was stirred at 0° C. for 30 minutes. Aluminum trichloride (1.57 g, 3 Eq, 11.8 mmol) was added and the reaction mixture was heated to 105° C. for 45 minutes. The reaction mixture was cooled some and slowly poured onto ice water (50 mL). The aqueous layer was extracted with EtOAc (3×50 mL) and concentrated in vacuo to afford crude material. The crude material was purified by silica gel chromatography (0ã100% 10% MeOH in EtOAc/hexanes) to afford 6-fluoro-5-(2-fluorophenoxy)-3-hydroxy-4H-benzo[e][1,2,4]thiadiazine 1,1-dioxide INT 1C (590 mg, 1.81 mmol, 46% yield)

### Step 1-4. Synthesis of 3-chloro-6-fluoro-5-(2-fluorophenoxy)-4H-benzo[e][1,2,4]thiadiazine 1,1-dioxide (INT 1D)

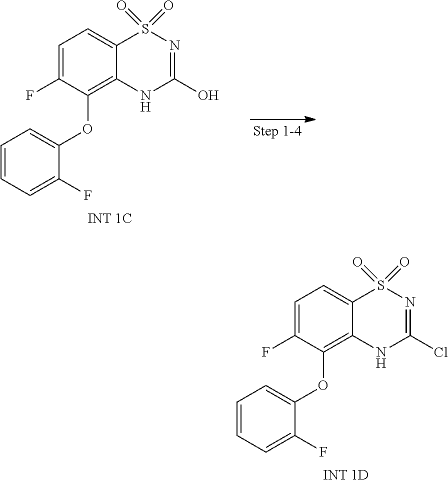 [SMILES: O=S1(=O)N=C(O)Nc2c1ccc(F)c2Oc1ccccc1F ; O=S1(=O)N=C(Cl)Nc2c1ccc(F)c2Oc1ccccc1F]

To a stirring solution of INT 1C (590 mg, 1 Eq, 1.81 mmol) was added Phosphorus oxychloride (11.1 g, 6.67 mL, 40 Eq, 72.3 mmol) and N,N-diethylaniline- (270 mg, 289 μL, 1 Eq, 1.81 mmol). The reaction mixture was stirred and heated at 125° C. for 18 h. The reaction mixture was concentrated in vacuo and purified by silica gel chromatography (0 to 100% 10% MeOH in EtOAc/hexanes) to afford 3-chloro-6-fluoro-5-(2-fluorophenoxy)-4H-benzo[e][1,2,4]thiadiazine 1,1-dioxide INT 1D (549.8 mg, 1.595 mmol, 88% yield).

### Step 1-5. Synthesis of 6-fluoro-5-(2-fluorophenoxy)-3-(((3-fluoropyridin-2-yl)methyl)amino)-4H-benzo[e][1,2,4]thiadiazine 1,1-dioxide (Compound 1-15)

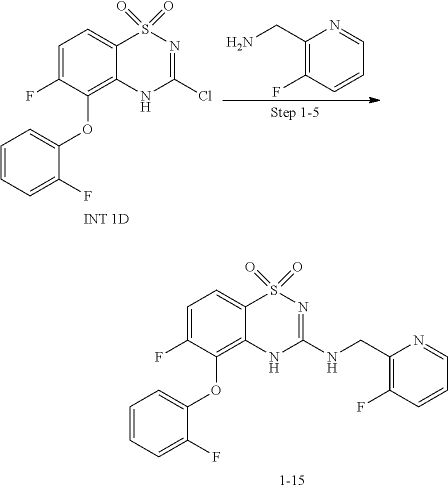 [SMILES: O=S1(=O)N=C(Cl)Nc2c1ccc(F)c2Oc1ccccc1F ; NCc1ncccc1F ; O=S1(=O)N=C(NCc2ncccc2F)Nc2c1ccc(F)c2Oc1ccccc1F]

To INT 1D (80 mg, 1 Eq, 0.23 mmol) in EtOH (2 mL) was added triethylamine (70 mg, 97 μL, 3 Eq, 0.70 mmol) and (3-fluoropyridin-2-yl)methanamine (59 mg, 2 Eq, 0.46 mmol). The reaction vial was capped and heated at 65° C. for 15 h. The reaction mixture was concentrated in vacuo and purified by reversed phase HPLC (35-55% 0.1% formic acid in MeCN/0.1% formic acid in H₂O) that was lyophilized to afford 6-fluoro-5-(2-fluorophenoxy)-3-(((3-fluoropyridin-2-yl)methyl)amino)-4H-benzo[e][1,2,4]thiadiazine 1,1-dioxide 1-15 (19 mg, 43 μmol, 18%). LCMS (m/z) calculated for C₁₉H₁₃F₃N₄O₃S: 434.0; found 435.0 [M+H]+, tR=7.797 min (Escient Purity 1). ¹H NMR (400 MHz, DMSO-d6) δ 10.87 (s, 1H), 8.43 (d, J=4.6 Hz, 1H), 8.02 (s, 1H), 7.78 (t, J=9.2 Hz, 1H), 7.74-7.60 (m, 1H), 7.56-7.36 (m, 2H), 7.29 (t, J=9.7 Hz, 1H), 7.17 (dt, J=15.5, 6.8 Hz, 2H), 6.99 (t, J=8.2 Hz, 1H), 4.68 (d, J=4.7 Hz, 2H). ¹⁹F NMR (376 MHz, DMSO-d₆) δ−123.16, −126.49, −133.79.

### Synthesis of Scheme 1 Compounds

Alternatively for Step 1-4, the following compounds were synthesized without the addition of N,N-diethylaniline: 1-59, 1-60, 1-61, 1-64, 1-65, 1-66, 1-67, 1-89, 1-99, 1-100, 1-101, 1-102, 1-103, 1-104, 1-105, 1-106, 1-107, 1-108, 1-109, 1-110, 1-111, 1-112, 1-113, 1-114, 1-115, 1-116, 1-117, 1-118, 1-119, 1-120, 1-121, 1-122, 1-123, 1-124, 1-125, 1-126, 1-127, 1-128, 1-129, 1-130, 1-131, 1-132, 1-133, 1-134, 1-135, 1-136, 1-137, 1-138, 1-139, 1-140, 1-141, 1-142, 1-143, 1-144, 1-145, 1-146, 1-147, 1-148, 1-149, 1-150, 1-151, 1-152, 1-153, 1-163, 1-164.

Alternatively for Step 1-5, the following compounds were made using 3 eq of DIPEA in NMP at 90° C. for 3 h: 1-62, 1-63, 1-68, 1-69, 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-90, 1-91, 1-92, 1-93, 1-94, 1-95, 1-96.

Alternatively for Step 1-5, the following compounds were made using 3 eq of DIPEA in NMP at 120° C. for 3 h: 1-2, 1-3, 1-4, 1-5, 1-6, 1-7, 1-11, 1-12, 1-13, 1-14, 1-20, 1-21, 1-23, 1-24, 1-25, 1-26, 1-27, 1-28, 1-29, 1-30, 1-31, 1-32, 1-49, 1-52, 1-22, 1-101, 1-102, 1-103, 1-104, 1-105, 1-106, 1-107, 1-108, 1-109, 1-110, 1-111, 1-112, 1-113, 1-114, 1-115, 1-116, 1-117, 1-118, 1-119, 1-120, 1-121, 1-122, 1-123, 1-124, 1-125, 1-126, 1-127, 1-128, 1-129, 1-130, 1-131, 1-132, 1-133, 1-134, 1-135, 1-136, 1-139, 1-140, 1-141, 1-142, 1-143, 1-144, 1-145, 1-146, 1-147, 1-148, 1-149, 1-150, 1-151, 1-152, 1-153, 1-163, 1-164.

Alternatively for Step 1-5, the following compounds were made using 3 eq of DIPEA in NMP at 130° C. for 18 h: 1-200, 1-201, 1-202, 1-203, 1-204, 1-205, 1-206, 1-207, 1-208, 1-209, 1-234, 1-246, 1-247, 1-248, 1-249, 1-254, 1-259, 1-260, 1-261, 1-262, 1-268, 1-269, 1-270, 1-271, 1-272, 1-273, 1-274, 1-275, 1-276, 1-277, 1-278, 1-279.

Alternatively for Step 1-5, the following compounds were made using 3 eq of DIPEA in DMA at 130° C. for 16 h: 1-64, 1-65, 1-66, 1-67, 1-89.

Alternatively for Step 1-5, the following compounds were made using 3 eq of DIPEA in DMF at 120° C. for 2 h: 1-97, 1-98.

Alternatively for Step 1-5, the following compounds were made using 3 eq of DIPEA in THF at 70° C. for 18 h: 1-53, 1-59, 1-60, 1-61, 1-99, 1-100, 1-137, 1-138.

The compounds listed in Table 2 were made using the procedures of Scheme 1.

TABLE 2

| | | Purity | Exact | | | |
|---|---|---|---|---|---|---|
| Cpd | | RT | mass | obsvd | | |
| No. | Structure | (min) | (g/mol) | m/z | Ion | Mthd |
| 1-1 | 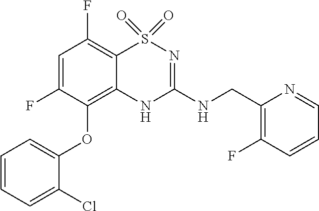 [SMILES: O=S1(=O)N=C(NCc2ncccc2F)Nc2c(Oc3ccccc3Cl)c(F)cc(F)c21] | 8.359 | 468 | 469 | [M + H]⁺ | 3 |
| 1-2 | 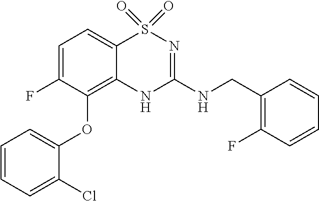 [SMILES: O=S1(=O)N=C(NCc2ccccc2F)Nc2c1ccc(F)c2Oc1ccccc1Cl] | 9.068 | 449 | 450 | [M + H]⁺ | 3 |
| 1-3 | 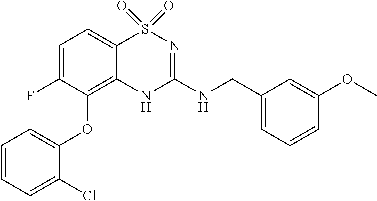 [SMILES: COc1cccc(CNC2=NS(=O)(=O)c3ccc(F)c(Oc4ccccc4Cl)c3N2)c1] | 9.096 | 461 | 462 | [M + H]⁺ | 3 |
| 1-4 | 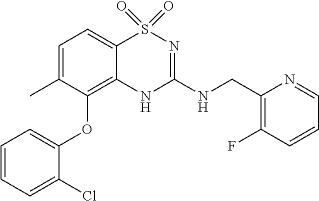 [SMILES: Cc1ccc2c(c1Oc1ccccc1Cl)NC(NCc1ncccc1F)=NS2(=O)=O] | 8.27 | 446 | 447 | [M + H]⁺ | 3 |
| 1-5 | 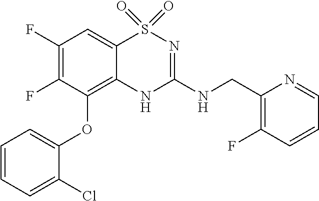 [SMILES: O=S1(=O)N=C(NCc2ncccc2F)Nc2c1cc(F)c(F)c2Oc1ccccc1Cl] | 8.673 | 468 | 469 | [M + H]⁺ | 3 |
| 1-6 | 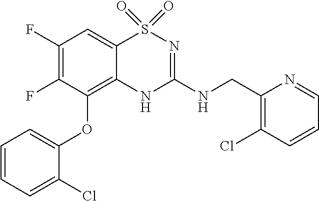 [SMILES: O=S1(=O)N=C(NCc2ncccc2Cl)Nc2c1cc(F)c(F)c2Oc1ccccc1Cl] | 9.262 | 484 | 486 | [M + H]⁺ | 3 |
| 1-7 | 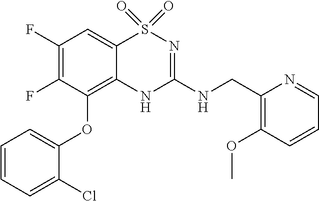 [SMILES: COc1cccnc1CNC1=NS(=O)(=O)c2cc(F)c(F)c(Oc3ccccc3Cl)c2N1] | 8.145 | 480 | 481 | [M + H]⁺ | 3 |
| 1-8 | 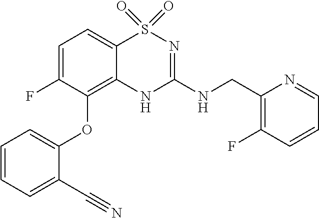 [SMILES: N#Cc1ccccc1Oc1c(F)ccc2c1NC(NCc1ncccc1F)=NS2(=O)=O] | 7.35 | 441 | 442 | [M + H]⁺ | 3 |
| 1-9 | 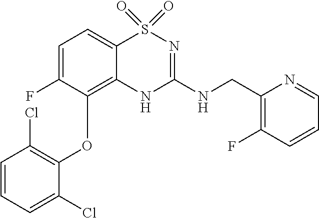 [SMILES: O=S1(=O)N=C(NCc2ncccc2F)Nc2c1ccc(F)c2Oc1c(Cl)cccc1Cl] | 8.394 | 484 | 485 | [M + H]⁺ | 3 |
| 1-10 | 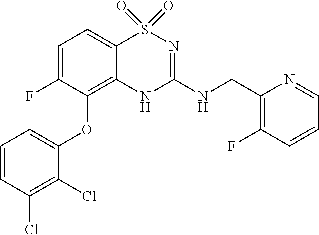 [SMILES: O=S1(=O)N=C(NCc2ncccc2F)Nc2c1ccc(F)c2Oc1cccc(Cl)c1Cl] | 9.023 | 484 | 485 | [M + H]⁺ | 3 |
| 1-11 | 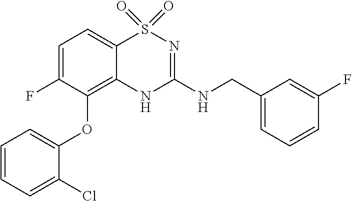 [SMILES: O=S1(=O)N=C(NCc2cccc(F)c2)Nc2c1ccc(F)c2Oc1ccccc1Cl] | 9.159 | 449 | 450 | [M + H]⁺ | 3 |
| 1-12 | 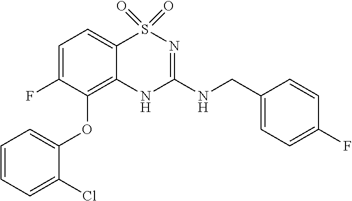 [SMILES: O=S1(=O)N=C(NCc2ccc(F)cc2)Nc2c1ccc(F)c2Oc1ccccc1Cl] | 9.212 | 449 | 450 | [M + H]⁺ | 3 |
| 1-13 | 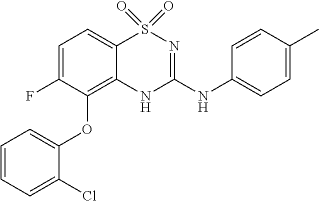 [SMILES: Cc1ccc(NC2=NS(=O)(=O)c3ccc(F)c(Oc4ccccc4Cl)c3N2)cc1] | 9.445 | 431 | 432 | [M + H]⁺ | 3 |
| 1-14 | 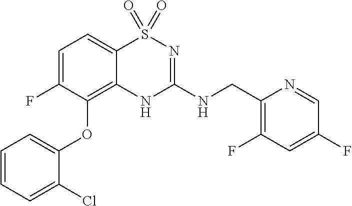 [SMILES: O=S1(=O)N=C(NCc2ncc(F)cc2F)Nc2c1ccc(F)c2Oc1ccccc1Cl] | 8.398 | 468 | 469 | [M + H]⁺ | 3 |
| 1-15 | 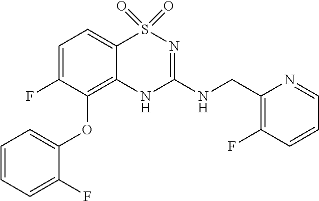 [SMILES: O=S1(=O)N=C(NCc2ncccc2F)Nc2c1ccc(F)c2Oc1ccccc1F] | 7.797 | 434 | 435 | [M + H]⁺ | 3 |
| 1-16 | 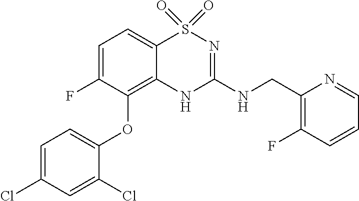 [SMILES: O=S1(=O)N=C(NCc2ncccc2F)Nc2c1ccc(F)c2Oc1ccc(Cl)cc1Cl] | 9.161 | 484 | 485 | [M + H]⁺ | 3 |
| 1-17 | 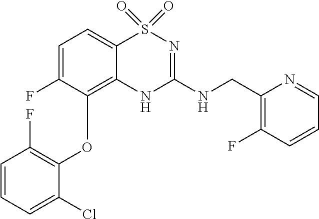 [SMILES: O=S1(=O)N=C(NCc2ncccc2F)Nc2c1ccc(F)c2Oc1c(F)cccc1Cl] | 8.101 | 468 | 469 | [M + H]⁺ | 3 |
| 1-18 | 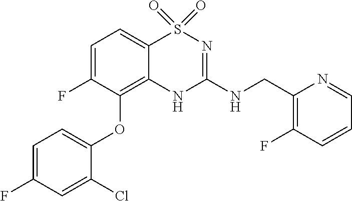 [SMILES: O=S1(=O)N=C(NCc2ncccc2F)Nc2c1ccc(F)c2Oc1ccc(F)cc1Cl] | 8.519 | 468 | 469 | [M + H]⁺ | 3 |
| 1-19 | 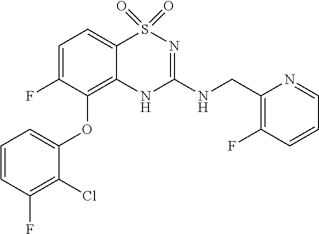 [SMILES: O=S1(=O)N=C(NCc2ncccc2F)Nc2c1ccc(F)c2Oc1cccc(F)c1Cl] | 8.521 | 468 | 469 | [M + H]⁺ | 3 |
| 1-20 | 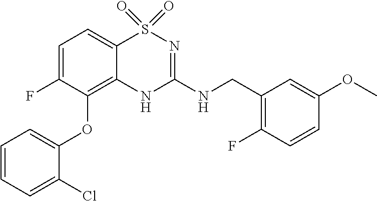 [SMILES: COc1ccc(F)c(CNC2=NS(=O)(=O)c3ccc(F)c(Oc4ccccc4Cl)c3N2)c1] | 9.296 | 479 | 480 | [M + H]⁺ | 3 |
| 1-21 | 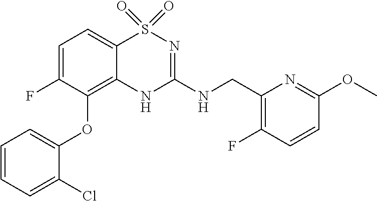 [SMILES: COc1ccc(F)c(CNC2=NS(=O)(=O)c3ccc(F)c(Oc4ccccc4Cl)c3N2)n1] | 9.162 | 480 | 481 | [M + H]⁺ | 3 |
| 1-22 | 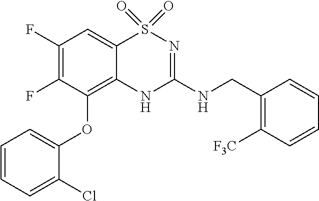 [SMILES: O=S1(=O)N=C(NCc2ccccc2C(F)(F)F)Nc2c1cc(F)c(F)c2Oc1ccccc1Cl] | 5.463 | 517 | 518 | [M + H]⁺ | 3 |
| 1-23 | 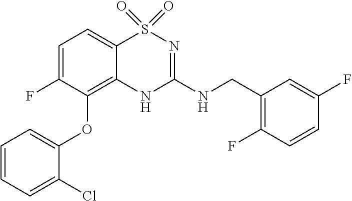 [SMILES: O=S1(=O)N=C(NCc2cc(F)ccc2F)Nc2c1ccc(F)c2Oc1ccccc1Cl] | 9.157 | 467 | 468 | [M + H]⁺ | 3 |
| 1-24 | 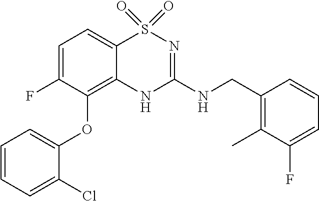 [SMILES: Cc1c(F)cccc1CNC1=NS(=O)(=O)c2ccc(F)c(Oc3ccccc3Cl)c2N1] | 9.477 | 463 | 464 | [M + H]⁺ | m 3 |
| 1-25 | 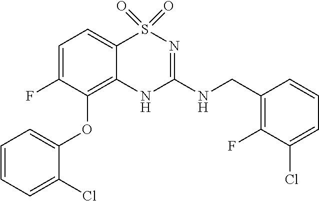 [SMILES: O=S1(=O)N=C(NCc2cccc(Cl)c2F)Nc2c1ccc(F)c2Oc1ccccc1Cl] | 9.566 | 483 | 484 | [M + H]⁺ | 3 |
| 1-26 | 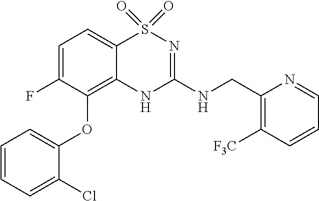 [SMILES: O=S1(=O)N=C(NCc2ncccc2C(F)(F)F)Nc2c1ccc(F)c2Oc1ccccc1Cl] | 9.2 | 500 | 501 | [M + H]⁺ | 3 |
| 1-27 | 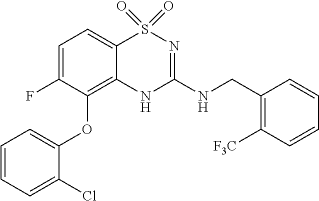 [SMILES: O=S1(=O)N=C(NCc2ccccc2C(F)(F)F)Nc2c1ccc(F)c2Oc1ccccc1Cl] | 9.687 | 499 | 500 | [M + H]⁺ | 3 |
| 1-28 | 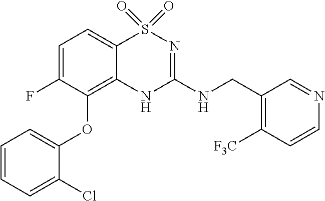 [SMILES: O=S1(=O)N=C(NCc2cnccc2C(F)(F)F)Nc2c1ccc(F)c2Oc1ccccc1Cl] | 8.401 | 500 | 501 | [M + H]⁺ | 3 |
| 1-29 | 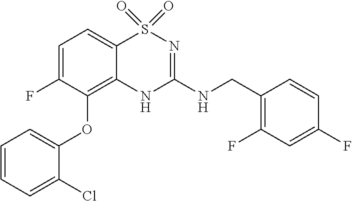 [SMILES: O=S1(=O)N=C(NCc2ccc(F)cc2F)Nc2c1ccc(F)c2Oc1ccccc1Cl] | 9.22 | 467 | 468 | [M + H]⁺ | 3 |
| 1-30 | 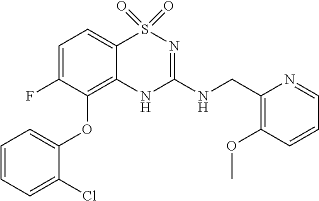 [SMILES: COc1cccnc1CNC1=NS(=O)(=O)c2ccc(F)c(Oc3ccccc3Cl)c2N1] | 7.669 | 462 | 463 | [M + H]⁺ | 3 |
| 1-31 | 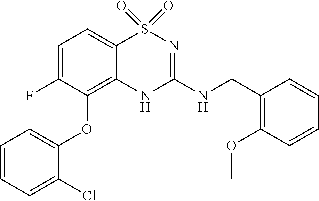 [SMILES: COc1ccccc1CNC1=NS(=O)(=O)c2ccc(F)c(Oc3ccccc3Cl)c2N1] | 9.323 | 461 | 462 | [M + H]⁺ | 3 |
| 1-32 | 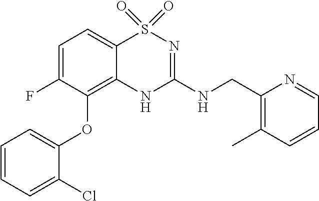 [SMILES: Cc1cccnc1CNC1=NS(=O)(=O)c2ccc(F)c(Oc3ccccc3Cl)c2N1] | 6.562 | 446 | 447 | [M + H]⁺ | 3 |
| 1-33 | 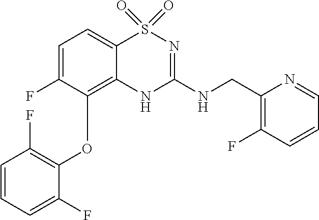 [SMILES: O=S1(=O)N=C(NCc2ncccc2F)Nc2c1ccc(F)c2Oc1c(F)cccc1F] | 7.735 | 452 | 453 | [M + H]⁺ | 3 |
| 1-34 | 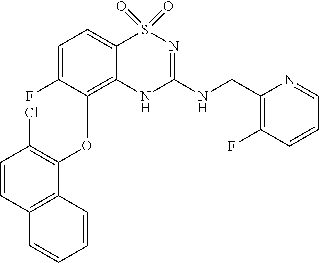 [SMILES: O=S1(=O)N=C(NCc2ncccc2F)Nc2c1ccc(F)c2Oc1c(Cl)ccc2ccccc12] | 9.012 | 500 | 501 | [M + H]⁺ | 3 |
| 1-35 | 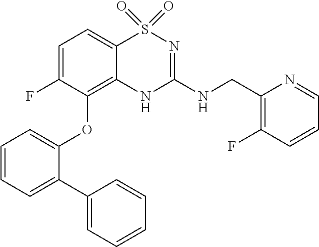 [SMILES: O=S1(=O)N=C(NCc2ncccc2F)Nc2c1ccc(F)c2Oc1ccccc1-c1ccccc1] | 9.294 | 492 | 493 | [M + H]⁺ | 3 |
| 1-36 | 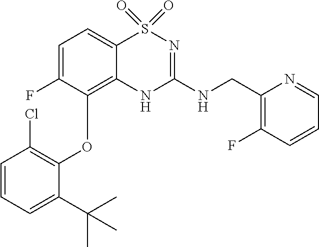 [SMILES: CC(C)(C)c1cccc(Cl)c1Oc1c(F)ccc2c1NC(NCc1ncccc1F)=NS2(=O)=O] | 9.861 | 506 | 507 | [M + H]⁺ | 3 |
| 1-37 | 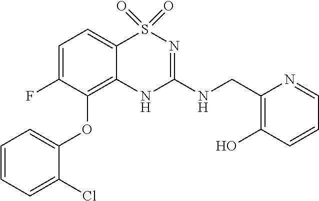 [SMILES: O=S1(=O)N=C(NCc2ncccc2O)Nc2c1ccc(F)c2Oc1ccccc1Cl] | 6.088 | 448 | 449 | [M + H]⁺ | 3 |
| 1-38 | 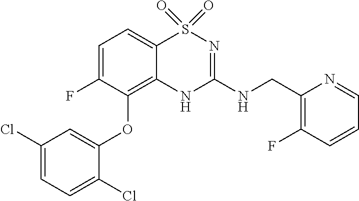 [SMILES: O=S1(=O)N=C(NCc2ncccc2F)Nc2c1ccc(F)c2Oc1cc(Cl)ccc1Cl] | 8.814 | 484 | 485 | [M + H]⁺ | 3 |
| 1-39 | 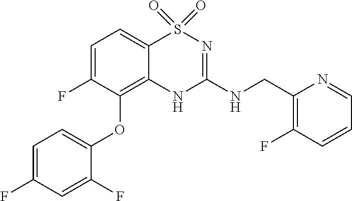 [SMILES: O=S1(=O)N=C(NCc2ncccc2F)Nc2c1ccc(F)c2Oc1ccc(F)cc1F] | 8.05 | 452 | 453 | [M + H]⁺ | 3 |
| 1-40 | 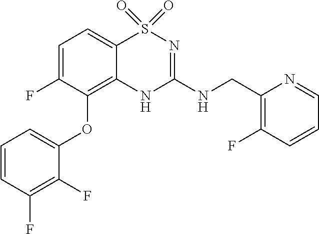 [SMILES: O=S1(=O)N=C(NCc2ncccc2F)Nc2c1ccc(F)c2Oc1cccc(F)c1F] | 8.126 | 452 | 453 | [M + H]⁺ | 3 |
| 1-41 | 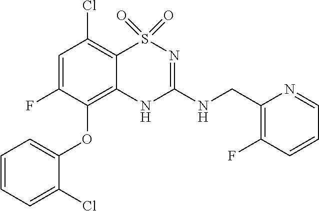 [SMILES: O=S1(=O)N=C(NCc2ncccc2F)Nc2c(Oc3ccccc3Cl)c(F)cc(Cl)c21] | 8.812 | 484 | 485 | [M + H]⁺ | 3 |
| 1-42 | 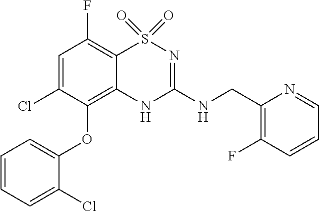 [SMILES: O=S1(=O)N=C(NCc2ncccc2F)Nc2c(Oc3ccccc3Cl)c(Cl)cc(F)c21] | 9.014 | 484 | 485 | [M + H]⁺ | 3 |
| 1-43 | 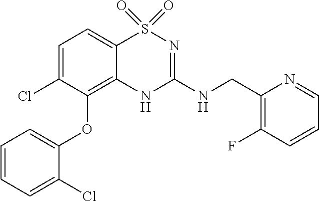 [SMILES: O=S1(=O)N=C(NCc2ncccc2F)Nc2c1ccc(Cl)c2Oc1ccccc1Cl] | 8.824 | 466 | 467 | [M + H]⁺ | 3 |
| 1-44 | 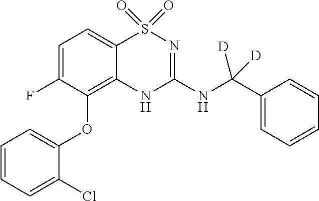 [SMILES: [2H]C([2H])(NC1=NS(=O)(=O)c2ccc(F)c(Oc3ccccc3Cl)c2N1)c1ccccc1] | 9.091 | 433 | 434 | [M + H]⁺ | 3 |
| 1-45 | 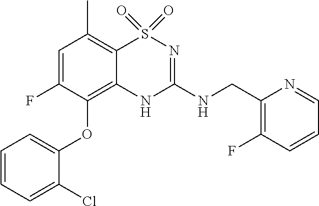 [SMILES: Cc1cc(F)c(Oc2ccccc2Cl)c2c1S(=O)(=O)N=C(NCc1ncccc1F)N2] | 8.796 | 464 | 465 | [M + H]⁺ | 3 |
| 1-46 | 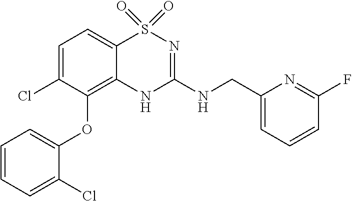 [SMILES: O=S1(=O)N=C(NCc2cccc(F)n2)Nc2c1ccc(Cl)c2Oc1ccccc1Cl] | 8.966 | 466 | 467 | [M + H]⁺ | 3 |
| 1-47 | 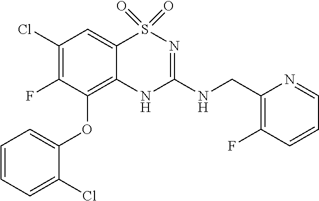 [SMILES: O=S1(=O)N=C(NCc2ncccc2F)Nc2c1cc(Cl)c(F)c2Oc1ccccc1Cl] | 8.303 | 484 | 485 | [M + H]⁺ | 3 |
| 1-48 | 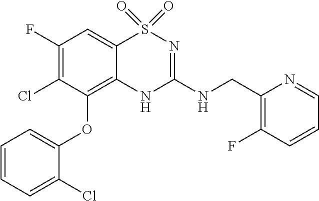 [SMILES: O=S1(=O)N=C(NCc2ncccc2F)Nc2c1cc(F)c(Cl)c2Oc1ccccc1Cl] | 9.263 | 484 | 485 | [M + H]⁺ | 3 |
| 1-49 | 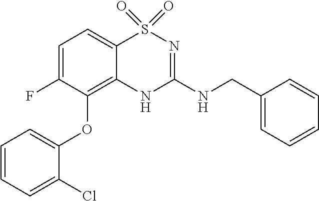 [SMILES: O=S1(=O)N=C(NCc2ccccc2)Nc2c1ccc(F)c2Oc1ccccc1Cl] | 9.077 | 431 | 432 | [M + H]⁺ | 3 |
| 1-50 | 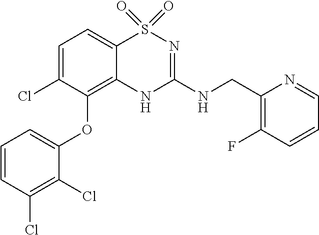 [SMILES: O=S1(=O)N=C(NCc2ncccc2F)Nc2c1ccc(Cl)c2Oc1cccc(Cl)c1Cl] | 9.606 | 500 | 501 | [M + H]⁺ | 3 |
| 1-51 | 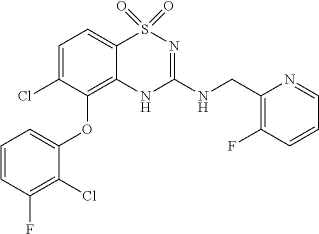 [SMILES: O=S1(=O)N=C(NCc2ncccc2F)Nc2c1ccc(Cl)c2Oc1cccc(F)c1Cl] | 9.062 | 484 | 485 | [M + H]⁺ | 3 |
| 1-52 | 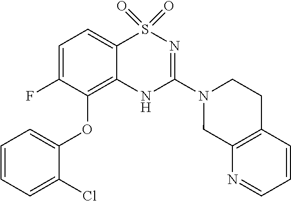 [SMILES: O=S1(=O)N=C(N2CCc3cccnc3C2)Nc2c1ccc(F)c2Oc1ccccc1Cl] | 7.483 | 458 | 459 | [M + H]⁺ | 3 |
| 1-53 | 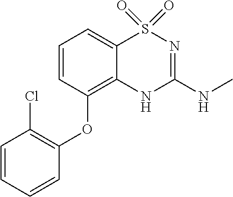 [SMILES: CNC1=NS(=O)(=O)c2cccc(Oc3ccccc3Cl)c2N1] | 6.915 | 337 | 338.2 | [M + H]⁺ | 3 |
| 1-54 | 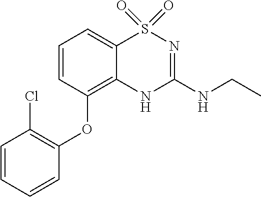 [SMILES: CCNC1=NS(=O)(=O)c2cccc(Oc3ccccc3Cl)c2N1] | 7.518 | 351 | 352.2 | [M + H]⁺ | 3 |
| 1-55 | 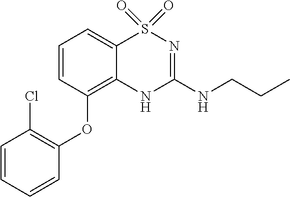 [SMILES: CCCNC1=NS(=O)(=O)c2cccc(Oc3ccccc3Cl)c2N1] | 8.107 | 365 | 366 | [M + H]⁺ | 3 |
| 1-56 | 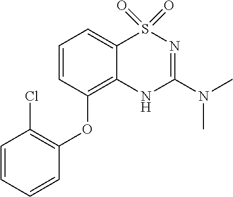 [SMILES: CN(C)C1=NS(=O)(=O)c2cccc(Oc3ccccc3Cl)c2N1] | 7.542 | 351 | 352 | [M + H]⁺ | 3 |
| 1-57 | 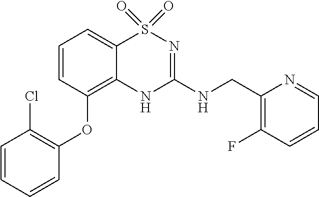 [SMILES: O=S1(=O)N=C(NCc2ncccc2F)Nc2c(Oc3ccccc3Cl)cccc21] | 7.946 | 432 | 433 | [M + H]⁺ | 3 |
| 1-58 | 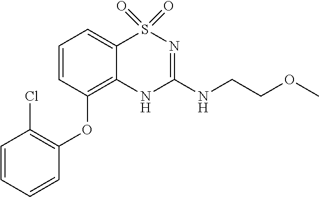 [SMILES: COCCNC1=NS(=O)(=O)c2cccc(Oc3ccccc3Cl)c2N1] | 7.368 | 381 | 382 | [M + H]⁺ | 3 |
| 1-59 | 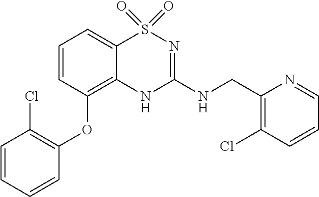 [SMILES: O=S1(=O)N=C(NCc2ncccc2Cl)Nc2c(Oc3ccccc3Cl)cccc21] | 8.549 | 448 | 449.2 | [M + H]⁺ | 3 |
| 1-60 | 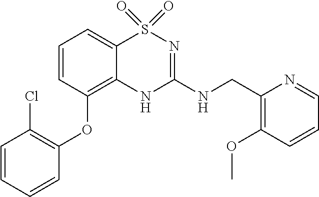 [SMILES: COc1cccnc1CNC1=NS(=O)(=O)c2cccc(Oc3ccccc3Cl)c2N1] | 7.031 | 444 | 445 | [M + H]⁺ | 3 |
| 1-61 | 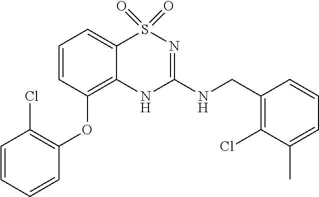 [SMILES: Cc1cccc(CNC2=NS(=O)(=O)c3cccc(Oc4ccccc4Cl)c3N2)c1Cl] | 9.656 | 461 | 462.2 | [M + H]⁺ | 3 |
| 1-62 | 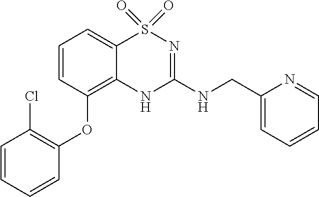 [SMILES: O=S1(=O)N=C(NCc2ccccn2)Nc2c(Oc3ccccc3Cl)cccc21] | 6.008 | 414 | 415.2 | [M + H]⁺ | 3 |
| 1-63 | 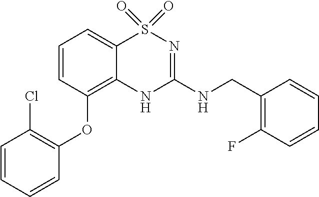 [SMILES: O=S1(=O)N=C(NCc2ccccc2F)Nc2c(Oc3ccccc3Cl)cccc21] | 8.803 | 431 | 432 | [M + H]⁺ | 3 |
| 1-64 | 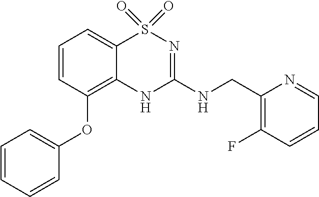 [SMILES: O=S1(=O)N=C(NCc2ncccc2F)Nc2c(Oc3ccccc3)cccc21] | 7.509 | 398 | 399 | [M + H]⁺ | 3 |
| 1-65 | 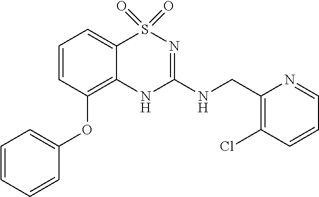 [SMILES: O=S1(=O)N=C(NCc2ncccc2Cl)Nc2c(Oc3ccccc3)cccc21] | 8.141 | 414 | 415 | [M + H]⁺ | 3 |
| 1-66 | 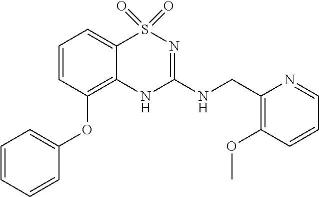 [SMILES: COc1cccnc1CNC1=NS(=O)(=O)c2cccc(Oc3ccccc3)c2N1] | 8.474 | 410 | 411 | [M + H]⁺ | 3 |
| 1-67 | 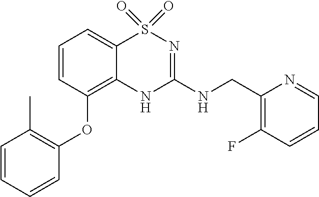 [SMILES: Cc1ccccc1Oc1cccc2c1NC(NCc1ncccc1F)=NS2(=O)=O] | 7.988, 8.059 | 412 | 413 | [M + H]⁺ | 3 |
| 1-68 | 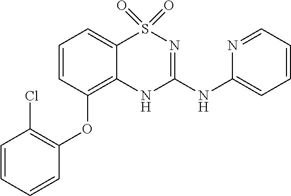 [SMILES: O=S1(=O)N=C(Nc2ccccn2)Nc2c(Oc3ccccc3Cl)cccc21] | 8.337 | 400 | 401 | [M + H]⁺ | 3 |
| 1-69 | 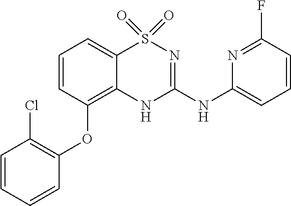 [SMILES: O=S1(=O)N=C(Nc2cccc(F)n2)Nc2c(Oc3ccccc3Cl)cccc21] | 8.907 | 418 | 419 | [M + H]⁺ | 3 |
| 1-70 | 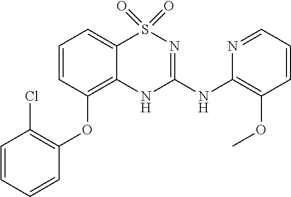 [SMILES: COc1cccnc1NC1=NS(=O)(=O)c2cccc(Oc3ccccc3Cl)c2N1] | 7.914 | 430 | 431 | [M + H]+ | 3 |
| 1-71 | 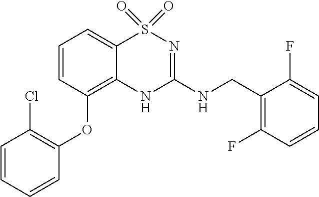 [SMILES: O=S1(=O)N=C(NCc2c(F)cccc2F)Nc2c(Oc3ccccc3Cl)cccc21] | 8.84 | 449 | 450 | [M + H]⁺ | 3 |
| 1-72 | 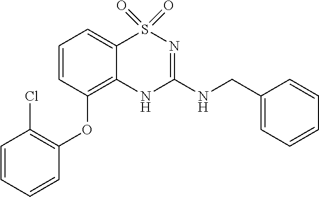 [SMILES: O=S1(=O)N=C(NCc2ccccc2)Nc2c(Oc3ccccc3Cl)cccc21] | 8.757 | 413 | 414 | [M + H]⁺ | 3 |
| 1-73 | 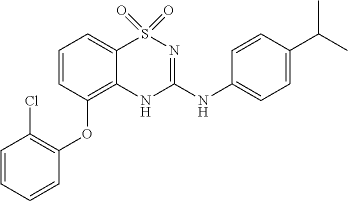 [SMILES: CC(C)c1ccc(NC2=NS(=O)(=O)c3cccc(Oc4ccccc4Cl)c3N2)cc1] | 10.119 | 441 | 442 | [M + H]⁺ | 3 |
| 1-74 | 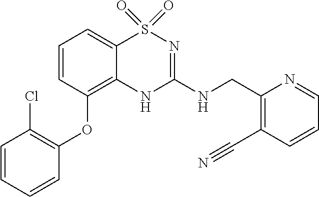 [SMILES: N#Cc1cccnc1CNC1=NS(=O)(=O)c2cccc(Oc3ccccc3Cl)c2N1] | 7.032 | 439 | 440 | [M + H]⁺ | 3 |
| 1-75 | 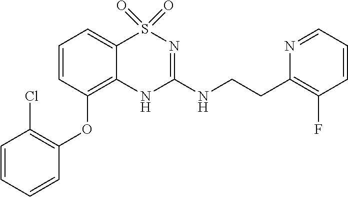 [SMILES: O=S1(=O)N=C(NCCc2ncccc2F)Nc2c(Oc3ccccc3Cl)cccc21] | 7.144 | 446 | 447 | [M + H]⁺ | 3 |
| 1-76 | 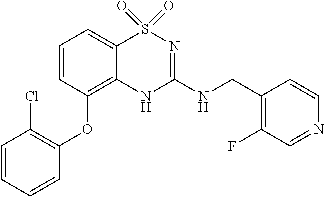 [SMILES: O=S1(=O)N=C(NCc2ccncc2F)Nc2c(Oc3ccccc3Cl)cccc21] | 7.085 | 432 | 433 | [M + H]⁺ | 3 |
| 1-77 | 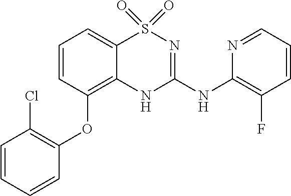 [SMILES: O=S1(=O)N=C(Nc2ncccc2F)Nc2c(Oc3ccccc3Cl)cccc21] | 7.605 | 418 | 419 | [M + H]⁺ | 3 |
| 1-78 | 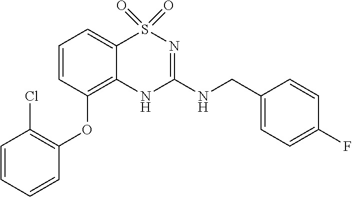 [SMILES: O=S1(=O)N=C(NCc2ccc(F)cc2)Nc2c(Oc3ccccc3Cl)cccc21] | 8.858 | 431 | 432 | [M + H]⁺ | 3 |
| 1-79 | 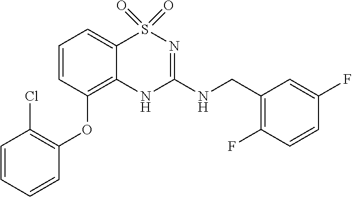 [SMILES: O=S1(=O)N=C(NCc2cc(F)ccc2F)Nc2c(Oc3ccccc3Cl)cccc21] | 8.932 | 449 | 450 | [M + H]⁺ | 3 |
| 1-80 | 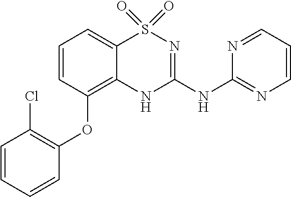 [SMILES: O=S1(=O)N=C(Nc2ncccn2)Nc2c(Oc3ccccc3Cl)cccc21] | 7.995 | 401 | 402 | [M + H]⁺ | 3 |
| 1-81 | 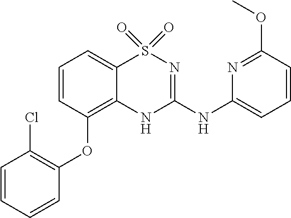 [SMILES: COc1cccc(NC2=NS(=O)(=O)c3cccc(Oc4ccccc4Cl)c3N2)n1] | 9.337 | 430 | 431 | [M + H]⁺ | 3 |
| 1-82 | 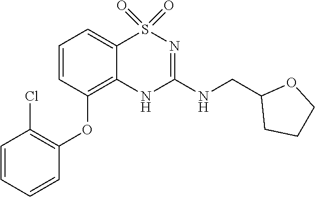 [SMILES: O=S1(=O)N=C(NCC2CCCO2)Nc2c(Oc3ccccc3Cl)cccc21] | 4.759 | 407 | 408 | [M + H]⁺ | 3 |
| 1-83 | 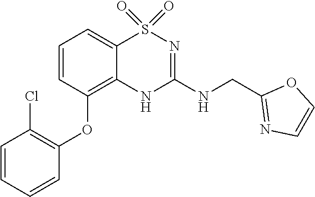 [SMILES: O=S1(=O)N=C(NCc2ncco2)Nc2c(Oc3ccccc3Cl)cccc21] | 6.992 | 404 | 405 | [M + H]⁺ | 3 |
| 1-84 | 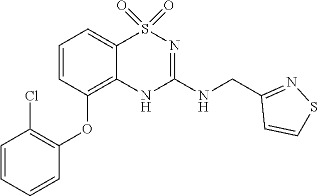 [SMILES: O=S1(=O)N=C(NCc2ccsn2)Nc2c(Oc3ccccc3Cl)cccc21] | 7.734 | 420 | 421 | [M + H]⁺ | 3 |
| 1-85 | 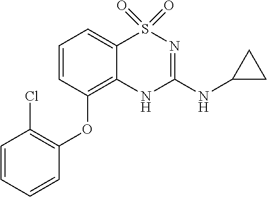 [SMILES: O=S1(=O)N=C(NC2CC2)Nc2c(Oc3ccccc3Cl)cccc21] | 7.708 | 363 | 364 | [M + H]⁺ | 3 |
| 1-86 | 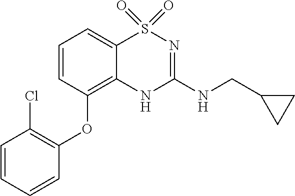 [SMILES: O=S1(=O)N=C(NCC2CC2)Nc2c(Oc3ccccc3Cl)cccc21] | 8.245 | 377 | 378 | [M + H]⁺ | 3 |
| 1-87 | 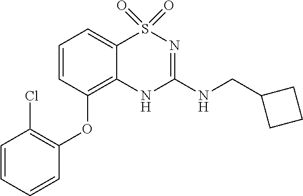 [SMILES: O=S1(=O)N=C(NCC2CCC2)Nc2c(Oc3ccccc3Cl)cccc21] | 8.855 | 391 | 392 | [M + H]⁺ | 3 |
| 1-88 | 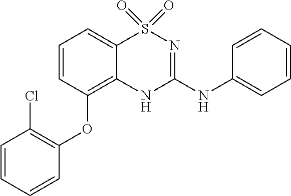 [SMILES: O=S1(=O)N=C(Nc2ccccc2)Nc2c(Oc3ccccc3Cl)cccc21] | 8.692 | 399 | 400 | [M + H]⁺ | 3 |
| 1-89 | 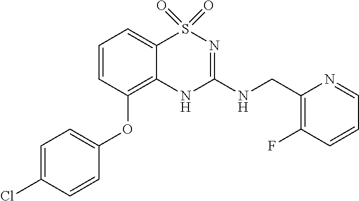 [SMILES: O=S1(=O)N=C(NCc2ncccc2F)Nc2c(Oc3ccc(Cl)cc3)cccc21] | 8.198 | 432 | 433 | [M + H]⁺ | 3 |
| 1-90 | 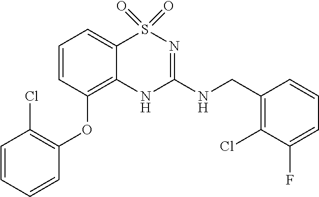 [SMILES: O=S1(=O)N=C(NCc2cccc(F)c2Cl)Nc2c(Oc3ccccc3Cl)cccc21] | 9.354 | 465 | 466 | [M + H]⁺ | 3 |
| 1-91 | 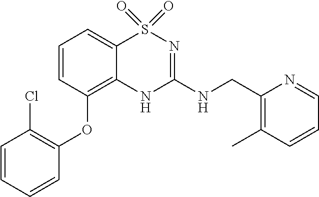 [SMILES: Cc1cccnc1CNC1=NS(=O)(=O)c2cccc(Oc3ccccc3Cl)c2N1] | 5.924 | 428 | 429 | [M + H]⁺ | 3 |
| 1-92 | 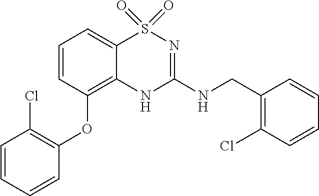 [SMILES: O=S1(=O)N=C(NCc2ccccc2Cl)Nc2c(Oc3ccccc3Cl)cccc21] | 9.227 | 447 | 448 | [M + H]⁺ | 3 |
| 1-93 | 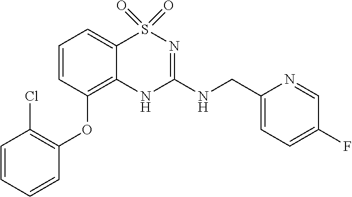 [SMILES: O=S1(=O)N=C(NCc2ccc(F)cn2)Nc2c(Oc3ccccc3Cl)cccc21] | 7.947 | 432 | 433 | [M + H]⁺ | 3 |
| 1-94 | 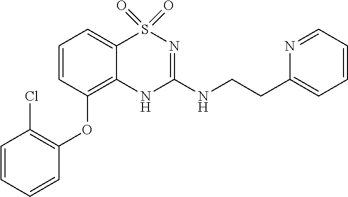 [SMILES: O=S1(=O)N=C(NCCc2ccccn2)Nc2c(Oc3ccccc3Cl)cccc21] | 5.1 | 428 | 429 | [M + H]⁺ | 3 |
| 1-95 | 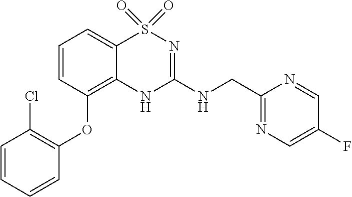 [SMILES: O=S1(=O)N=C(NCc2ncc(F)cn2)Nc2c(Oc3ccccc3Cl)cccc21] | 7.271 | 433 | 434 | [M + H]⁺ | 3 |
| 1-96 | 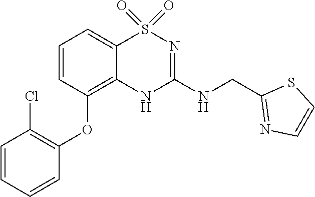 [SMILES: O=S1(=O)N=C(NCc2nccs2)Nc2c(Oc3ccccc3Cl)cccc21] | 7.2 | 420 | 421 | [M + H]⁺ | 3 |
| 1-97 | 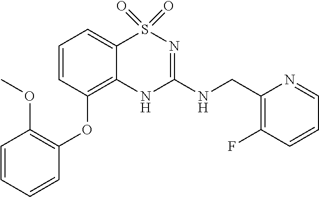 [SMILES: COc1ccccc1Oc1cccc2c1NC(NCc1ncccc1F)=NS2(=O)=O] | 7.312 | 428 | 429 | [M + H]⁺ | 3 |
| 1-98 | 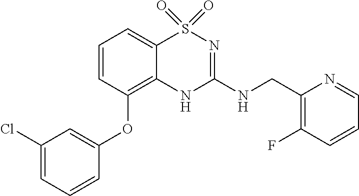 [SMILES: O=S1(=O)N=C(NCc2ncccc2F)Nc2c(Oc3cccc(Cl)c3)cccc21] | 8.159 | 432 | 433 | [M + H]⁺ | 3 |
| 1-99 | 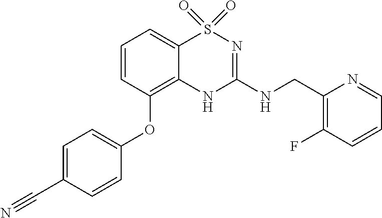 [SMILES: N#Cc1ccc(Oc2cccc3c2NC(NCc2ncccc2F)=NS3(=O)=O)cc1] | 6.93 | 423 | 424 | [M + H]⁺ | 3 |
| 1-100 | 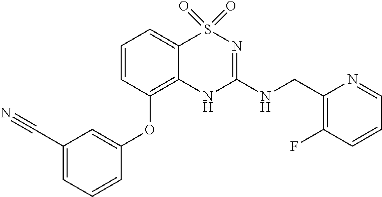 [SMILES: N#Cc1cccc(Oc2cccc3c2NC(NCc2ncccc2F)=NS3(=O)=O)c1] | 6.991 | 423 | 424 | [M + H]⁺ | 3 |
| 1-101 | 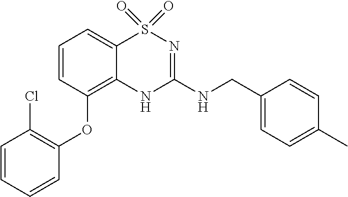 [SMILES: Cc1ccc(CNC2=NS(=O)(=O)c3cccc(Oc4ccccc4Cl)c3N2)cc1] | 9.149 | 427 | 428 | [M + H]⁺ | 3 |
| 1-102 | 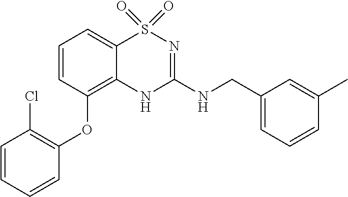 [SMILES: Cc1cccc(CNC2=NS(=O)(=O)c3cccc(Oc4ccccc4Cl)c3N2)c1] | 9.185 | 427 | 428 | [M + H]⁺ | 3 |
| 1-103 | 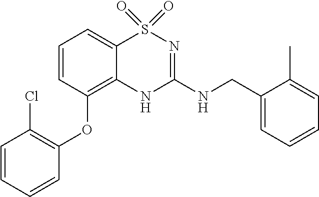 [SMILES: Cc1ccccc1CNC1=NS(=O)(=O)c2cccc(Oc3ccccc3Cl)c2N1] | 9.071 | 427 | 428 | [M + H]⁺ | 3 |
| 1-104 | 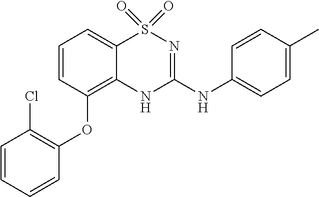 [SMILES: Cc1ccc(NC2=NS(=O)(=O)c3cccc(Oc4ccccc4Cl)c3N2)cc1] | 9.092 | 413 | 414 | [M + H]⁺ | 3 |
| 1-105 | 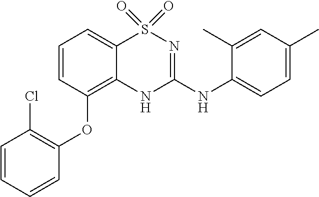 [SMILES: Cc1ccc(NC2=NS(=O)(=O)c3cccc(Oc4ccccc4Cl)c3N2)c(C)c1] | 9.281 | 427 | 428 | [M + H]⁺ | 3 |
| 1-106 | 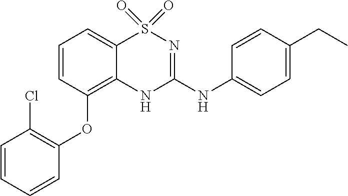 [SMILES: CCc1ccc(NC2=NS(=O)(=O)c3cccc(Oc4ccccc4Cl)c3N2)cc1] | 9.616 | 427 | 428 | [M + H]⁺ | 3 |
| 1-107 | 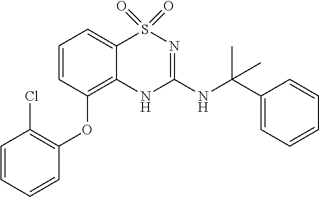 [SMILES: CC(C)(NC1=NS(=O)(=O)c2cccc(Oc3ccccc3Cl)c2N1)c1ccccc1] | 8.04 | 441 | 442.1 | [M + H]⁺ | 3 |
| 1-108 | 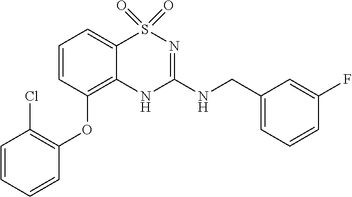 [SMILES: O=S1(=O)N=C(NCc2cccc(F)c2)Nc2c(Oc3ccccc3Cl)cccc21] | 8.734 | 431 | 432 | [M + H]⁺ | 3 |
| 1-109 | 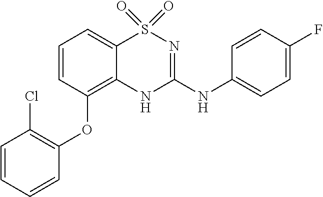 [SMILES: O=S1(=O)N=C(Nc2ccc(F)cc2)Nc2c(Oc3ccccc3Cl)cccc21] | 8.735 | 417 | 418 | [M + H]⁺ | 3 |
| 1-110 | 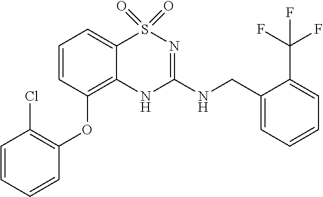 [SMILES: O=S1(=O)N=C(NCc2ccccc2C(F)(F)F)Nc2c(Oc3ccccc3Cl)cccc21] | 9.298 | 481 | 482 | [M + H]⁺ | 3 |
| 1-111 | 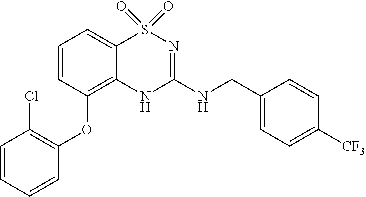 [SMILES: O=S1(=O)N=C(NCc2ccc(C(F)(F)F)cc2)Nc2c(Oc3ccccc3Cl)cccc21] | 9.478 | 481 | 482 | [M + H]⁺ | 3 |
| 1-112 | 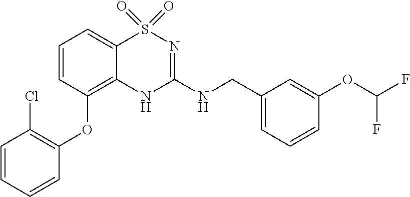 [SMILES: O=S1(=O)N=C(NCc2cccc(OC(F)F)c2)Nc2c(Oc3ccccc3Cl)cccc21] | 9.049 | 479 | 480 | [M + H]⁺ | 3 |
| 1-113 | 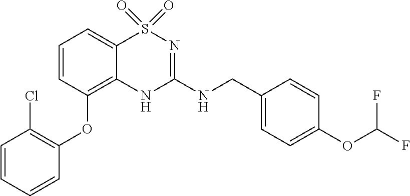 [SMILES: O=S1(=O)N=C(NCc2ccc(OC(F)F)cc2)Nc2c(Oc3ccccc3Cl)cccc21] | 9.034 | 479 | 480 | [M + H]⁺ | 3 |
| 1-114 | 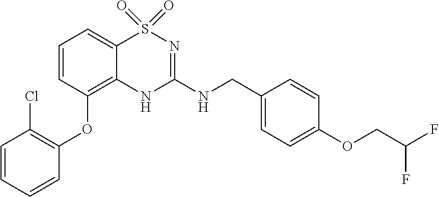 [SMILES: O=S1(=O)N=C(NCc2ccc(OCC(F)F)cc2)Nc2c(Oc3ccccc3Cl)cccc21] | 9.035 | 493 | 494 | [M + H]⁺ | 3 |
| 1-115 | 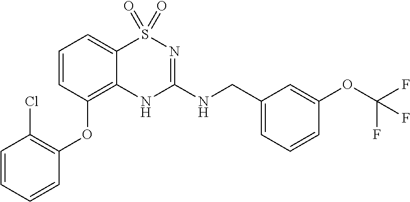 [SMILES: O=S1(=O)N=C(NCc2cccc(OC(F)(F)F)c2)Nc2c(Oc3ccccc3Cl)cccc21] | 9.646 | 497 | 498 | [M + H]⁺ | 3 |
| 1-116 | 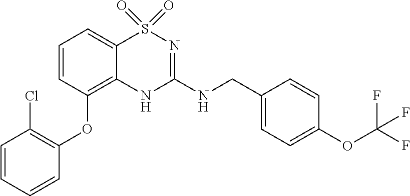 [SMILES: O=S1(=O)N=C(NCc2ccc(OC(F)(F)F)cc2)Nc2c(Oc3ccccc3Cl)cccc21] | 9.689 | 497 | 498 | [M + H]⁺ | 3 |
| 1-117 | 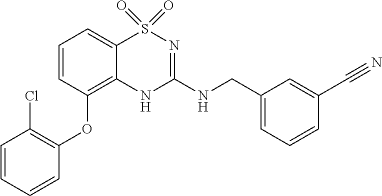 [SMILES: N#Cc1cccc(CNC2=NS(=O)(=O)c3cccc(Oc4ccccc4Cl)c3N2)c1] | 8.439 | 438 | 439 | [M + H]⁺ | 3 |
| 1-118 | 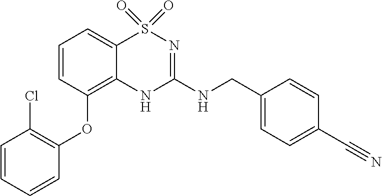 [SMILES: N#Cc1ccc(CNC2=NS(=O)(=O)c3cccc(Oc4ccccc4Cl)c3N2)cc1] | 8.222 | 438 | 439 | [M + H]⁺ | 3 |
| 1-119 | 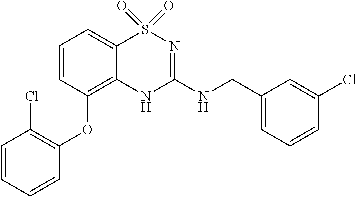 [SMILES: O=S1(=O)N=C(NCc2cccc(Cl)c2)Nc2c(Oc3ccccc3Cl)cccc21] | 9.413 | 447 | 448 | [M + H]⁺ | 3 |
| 1-120 | 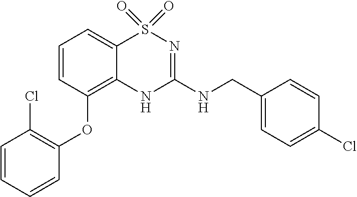 [SMILES: O=S1(=O)N=C(NCc2ccc(Cl)cc2)Nc2c(Oc3ccccc3Cl)cccc21] | 9.197 | 447 | 448 | [M + H]⁺ | 3 |
| 1-121 | 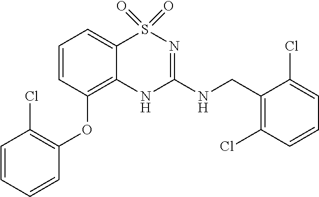 [SMILES: O=S1(=O)N=C(NCc2c(Cl)cccc2Cl)Nc2c(Oc3ccccc3Cl)cccc21] | 9.336 | 481 | 482 | [M + H]⁺ | 3 |
| 1-122 | 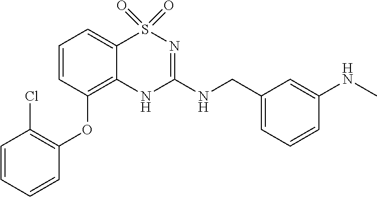 [SMILES: CNc1cccc(CNC2=NS(=O)(=O)c3cccc(Oc4ccccc4Cl)c3N2)c1] | 6.597 | 442 | 443 | [M + H]⁺ | 3 |
| 1-123 | 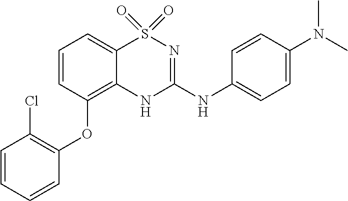 [SMILES: CN(C)c1ccc(NC2=NS(=O)(=O)c3cccc(Oc4ccccc4Cl)c3N2)cc1] | 6.743 | 442 | 443 | [M + H]⁺ | 3 |
| 1-124 | 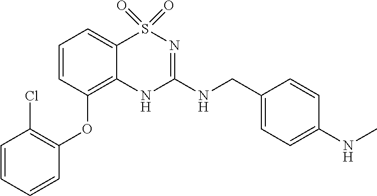 [SMILES: CNc1ccc(CNC2=NS(=O)(=O)c3cccc(Oc4ccccc4Cl)c3N2)cc1] | 6.186 | 442 | 443 | [M + H]⁺ | 3 |
| 1-125 | 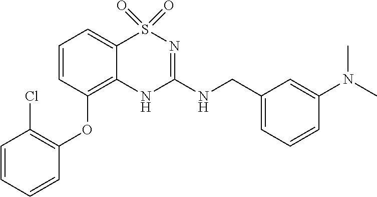 [SMILES: CN(C)c1cccc(CNC2=NS(=O)(=O)c3cccc(Oc4ccccc4Cl)c3N2)c1] | 6.801 | 456 | 457 | [M + H]⁺ | 3 |
| 1-126 | 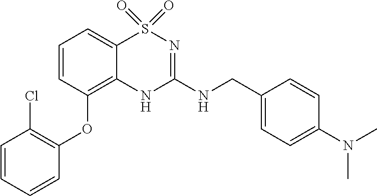 [SMILES: CN(C)c1ccc(CNC2=NS(=O)(=O)c3cccc(Oc4ccccc4Cl)c3N2)cc1] | 6.528 | 456 | 457 | [M + H]⁺ | 3 |
| 1-127 | 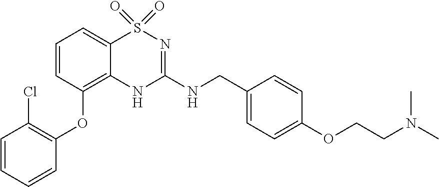 [SMILES: CN(C)CCOc1ccc(CNC2=NS(=O)(=O)c3cccc(Oc4ccccc4Cl)c3N2)cc1] | 5.771 | 500 | 501.2 | [M + H]⁺ | 3 |
| 1-128 | 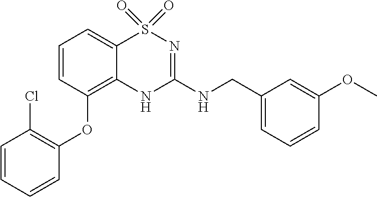 [SMILES: COc1cccc(CNC2=NS(=O)(=O)c3cccc(Oc4ccccc4Cl)c3N2)c1] | 8.722 | 443 | 444 | [M + H]⁺ | 3 |
| 1-129 | 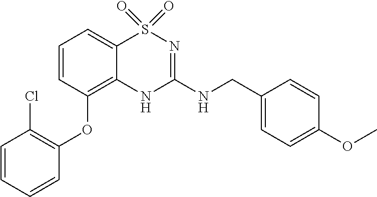 [SMILES: COc1ccc(CNC2=NS(=O)(=O)c3cccc(Oc4ccccc4Cl)c3N2)cc1] | 8.689 | 443 | 444 | [M + H]⁺ | 3 |
| 1-130 | 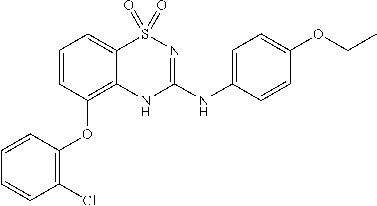 [SMILES: CCOc1ccc(NC2=NS(=O)(=O)c3cccc(Oc4ccccc4Cl)c3N2)cc1] | 9.082 | 443 | 444 | [M + H]⁺ | 3 |
| 1-131 | 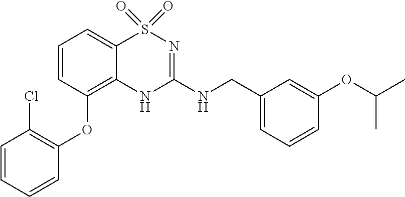 [SMILES: CC(C)Oc1cccc(CNC2=NS(=O)(=O)c3cccc(Oc4ccccc4Cl)c3N2)c1] | 9.604 | 471 | 472 | [M + H]⁺ | 3 |
| 1-132 | 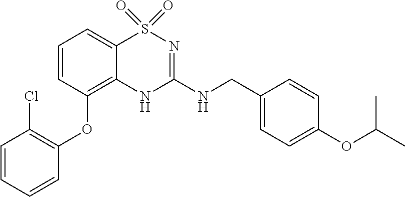 [SMILES: CC(C)Oc1ccc(CNC2=NS(=O)(=O)c3cccc(Oc4ccccc4Cl)c3N2)cc1] | 9.607 | 471 | 472 | [M + H]⁺ | 3 |
| 1-133 | 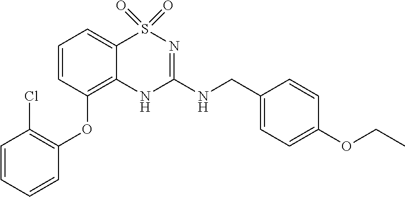 [SMILES: CCOc1ccc(CNC2=NS(=O)(=O)c3cccc(Oc4ccccc4Cl)c3N2)cc1] | 9.19 | 457 | 458 | [M + H]⁺ | 3 |
| 1-134 | 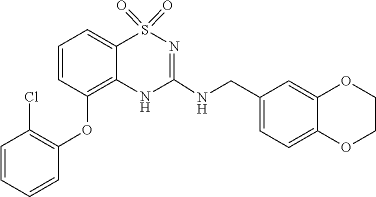 [SMILES: O=S1(=O)N=C(NCc2ccc3c(c2)OCCO3)Nc2c(Oc3ccccc3Cl)cccc21] | 8.584 | 471 | 472 | [M + H]⁺ | 3 |
| 1-135 | 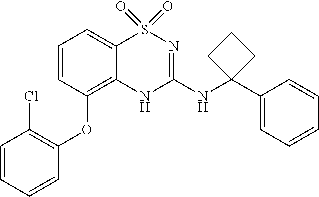 [SMILES: O=S1(=O)N=C(NC2(c3ccccc3)CCC2)Nc2c(Oc3ccccc3Cl)cccc21] | 9.584 | 453 | 454 | [M + H]⁺ | 3 |
| 1-136 | 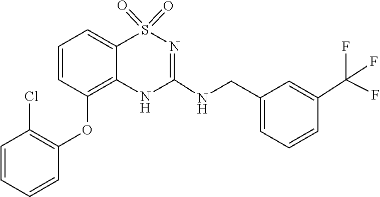 [SMILES: O=S1(=O)N=C(NCc2cccc(C(F)(F)F)c2)Nc2c(Oc3ccccc3Cl)cccc21] | 9.412 | 481 | 482 | [M + H]⁺ | 3 |
| 1-137 | 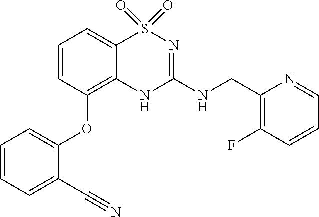 [SMILES: N#Cc1ccccc1Oc1cccc2c1NC(NCc1ncccc1F)=NS2(=O)=O] | 6.827 | 423 | 424 | [M + H]⁺ | 3 |
| 1-138 | 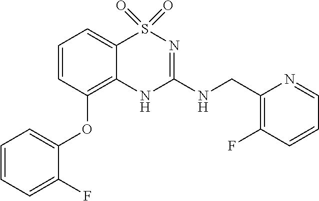 [SMILES: O=S1(=O)N=C(NCc2ncccc2F)Nc2c(Oc3ccccc3F)cccc21] | 7.336 | 416 | 417 | [M + H]⁺ | 3 |
| 1-139 | 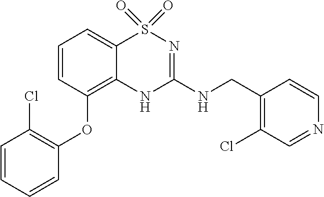 [SMILES: O=S1(=O)N=C(NCc2ccncc2Cl)Nc2c(Oc3ccccc3Cl)cccc21] | 7.4 | 448 | 449 | [M + H]⁺ | 3 |
| 1-140 | 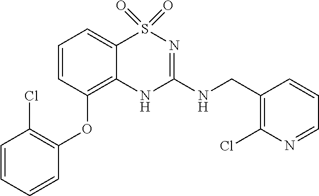 [SMILES: O=S1(=O)N=C(NCc2cccnc2Cl)Nc2c(Oc3ccccc3Cl)cccc21] | 7.746 | 448 | 449 | [M + H]⁺ | 3 |
| 1-141 | 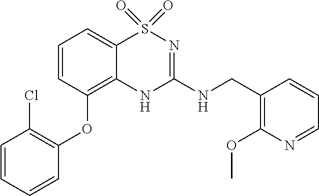 [SMILES: COc1ncccc1CNC1=NS(=O)(=O)c2cccc(Oc3ccccc3Cl)c2N1] | 8.039 | 444 | 445 | [M + H]⁺ | 3 |
| 1-142 | 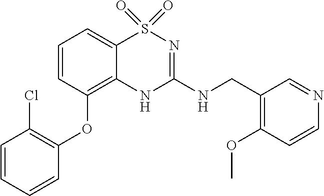 [SMILES: COc1ccncc1CNC1=NS(=O)(=O)c2cccc(Oc3ccccc3Cl)c2N1] | 5.623 | 444 | 445 | [M + H]⁺ | 3 |
| 1-143 | 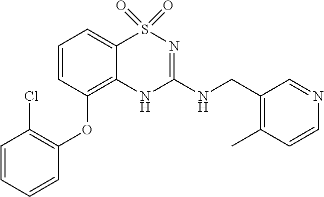 [SMILES: Cc1ccncc1CNC1=NS(=O)(=O)c2cccc(Oc3ccccc3Cl)c2N1] | 5.151 | 428 | 429 | [M + H]⁺ | 3 |
| 1-144 | 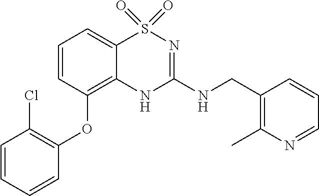 [SMILES: Cc1ncccc1CNC1=NS(=O)(=O)c2cccc(Oc3ccccc3Cl)c2N1] | 5.175 | 428 | 429 | [M + H]⁺ | 3 |
| 1-145 | 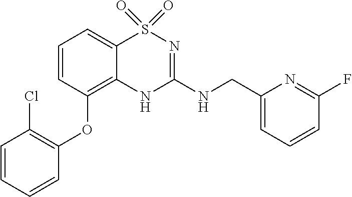 [SMILES: O=S1(=O)N=C(NCc2cccc(F)n2)Nc2c(Oc3ccccc3Cl)cccc21] | 8.075 | 432 | 433 | [M + H]⁺ | 3 |
| 1-146 | 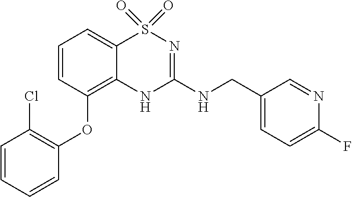 [SMILES: O=S1(=O)N=C(NCc2ccc(F)nc2)Nc2c(Oc3ccccc3Cl)cccc21] | 7.77 | 432 | 433 | [M + H]⁺ | 3 |
| 1-147 | 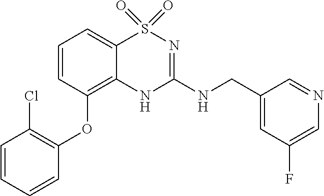 [SMILES: O=S1(=O)N=C(NCc2cncc(F)c2)Nc2c(Oc3ccccc3Cl)cccc21] | 7.429 | 432 | 433 | [M + H]⁺ | 3 |
| 1-148 | 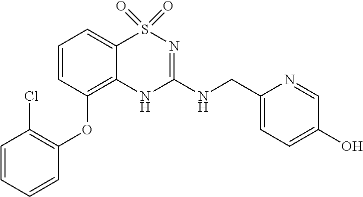 [SMILES: O=S1(=O)N=C(NCc2ccc(O)cn2)Nc2c(Oc3ccccc3Cl)cccc21] | 5.645 | 430 | 431 | [M + H]⁺ | 3 |
| 1-149 | 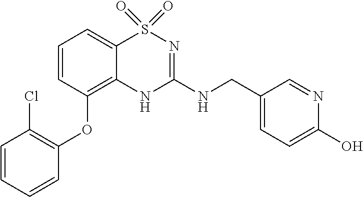 [SMILES: O=S1(=O)N=C(NCc2ccc(O)nc2)Nc2c(Oc3ccccc3Cl)cccc21] | 5.944 | 430 | 431 | [M + H]⁺ | 3 |
| 1-150 | 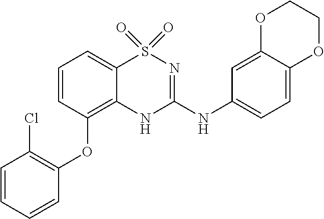 [SMILES: O=S1(=O)N=C(Nc2ccc3c(c2)OCCO3)Nc2c(Oc3ccccc3Cl)cccc21] | 8.553 | 457 | 458 | [M + H]⁺ | 3 |
| 1-151 | 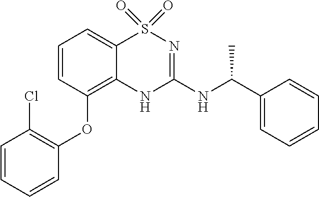 [SMILES: C[C@@H](NC1=NS(=O)(=O)c2cccc(Oc3ccccc3Cl)c2N1)c1ccccc1] | 9.083 | 427 | 428 | [M + H]⁺ | 3 |
| 1-152 | 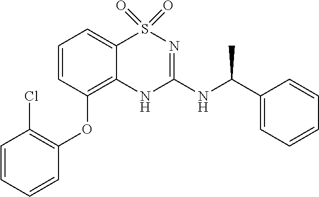 [SMILES: C[C@H](NC1=NS(=O)(=O)c2cccc(Oc3ccccc3Cl)c2N1)c1ccccc1] | 9.065 | 427 | 428 | [M + H]⁺ | 3 |
| 1-153 | 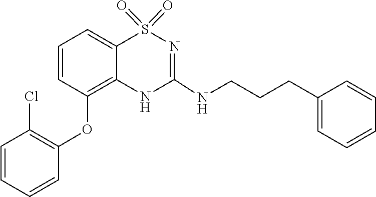 [SMILES: O=S1(=O)N=C(NCCCc2ccccc2)Nc2c(Oc3ccccc3Cl)cccc21] | 9.544 | 441 | 442 | [M + H]⁺ | 3 |
| 1-154 | 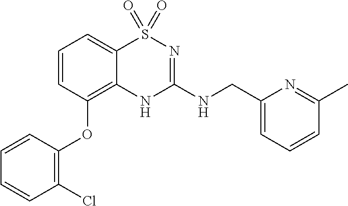 [SMILES: Cc1cccc(CNC2=NS(=O)(=O)c3cccc(Oc4ccccc4Cl)c3N2)n1] | 0.578 | 428 | 429.1 | [M + H]⁺ | 1 |
| 1-155 | 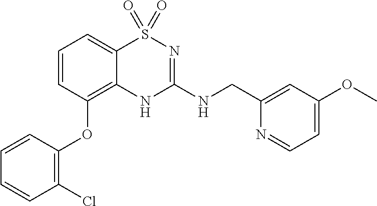 [SMILES: COc1ccnc(CNC2=NS(=O)(=O)c3cccc(Oc4ccccc4Cl)c3N2)c1] | 0.579 | 444 | 445.1 | [M + H]⁺ | 1 |
| 1-156 | 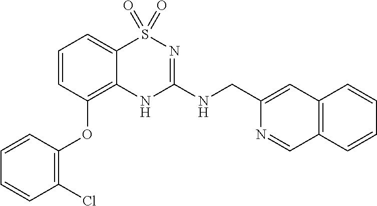 [SMILES: O=S1(=O)N=C(NCc2cc3ccccc3cn2)Nc2c(Oc3ccccc3Cl)cccc21] | 0.641 | 464 | 465.1 | [M + H]⁺ | 1 |
| 1-157 | 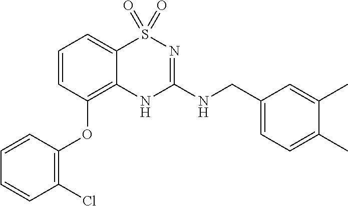 [SMILES: Cc1ccc(CNC2=NS(=O)(=O)c3cccc(Oc4ccccc4Cl)c3N2)cc1C] | 0.795 | 441 | 442.1 | [M + H]⁺ | 1 |
| 1-158 | 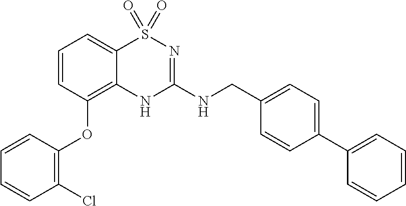 [SMILES: O=S1(=O)N=C(NCc2ccc(-c3ccccc3)cc2)Nc2c(Oc3ccccc3Cl)cccc21] | 0.828 | 489 | 490.1 | [M + H]⁺ | 1 |
| 1-159 | 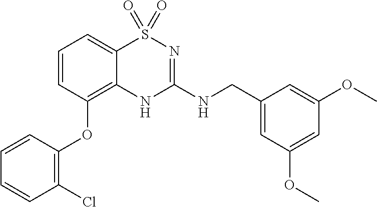 [SMILES: COc1cc(CNC2=NS(=O)(=O)c3cccc(Oc4ccccc4Cl)c3N2)cc(OC)c1] | 0.754 | 473 | 474.1 | [M + H]⁺ | 1 |
| 1-160 | 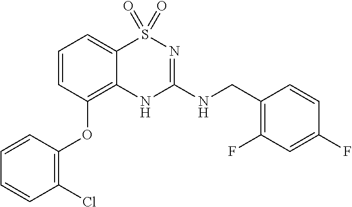 [SMILES: O=S1(=O)N=C(NCc2ccc(F)cc2F)Nc2c(Oc3ccccc3Cl)cccc21] | 0.765 | 449 | 450.1 | [M + H]⁺ | 1 |
| 1-161 | 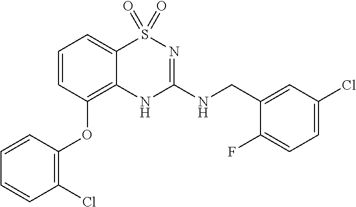 [SMILES: O=S1(=O)N=C(NCc2cc(Cl)ccc2F)Nc2c(Oc3ccccc3Cl)cccc21] | 0.780 | 465 | 468 | | 1 |
| 1-162 | 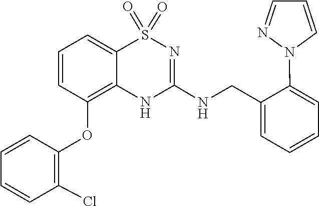 [SMILES: O=S1(=O)N=C(NCc2ccccc2-n2cccn2)Nc2c(Oc3ccccc3Cl)cccc21] | 0.540 | 479 | 480.2 | [M + H]⁺ | 1 |
| 1-163 | 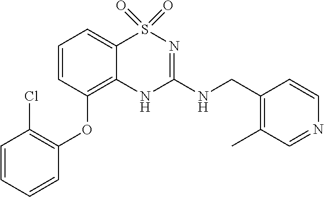 [SMILES: Cc1cnccc1CNC1=NS(=O)(=O)c2cccc(Oc3ccccc3Cl)c2N1] | 5.075 | 428 | 429 | [M + H]⁺ | 3 |
| 1-164 | 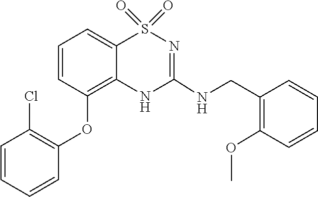 [SMILES: COc1ccccc1CNC1=NS(=O)(=O)c2cccc(Oc3ccccc3Cl)c2N1] | 9.32 | 443 | 444 | [M + H]⁺ | 3 |
| 1-165 | 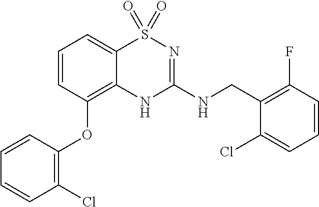 [SMILES: O=S1(=O)N=C(NCc2c(F)cccc2Cl)Nc2c(Oc3ccccc3Cl)cccc21] | 0.545 | 465 | 466 | [M + H]⁺ | 1 |
| 1-166 | 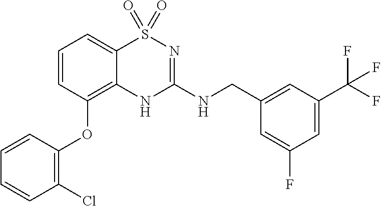 [SMILES: O=S1(=O)N=C(NCc2cc(F)cc(C(F)(F)F)c2)Nc2c(Oc3ccccc3Cl)cccc21] | 0.568 | 499 | 500.2 | [M + H]⁺ | 1 |
| 1-167 | 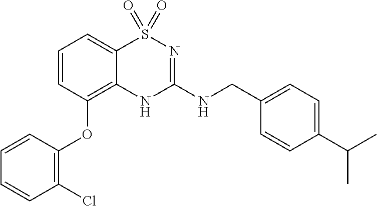 [SMILES: CC(C)c1ccc(CNC2=NS(=O)(=O)c3cccc(Oc4ccccc4Cl)c3N2)cc1] | 0.593 | 455 | 456.3 | [M + H]⁺ | 1 |
| 1-168 | 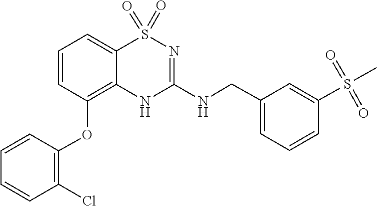 [SMILES: CS(=O)(=O)c1cccc(CNC2=NS(=O)(=O)c3cccc(Oc4ccccc4Cl)c3N2)c1] | 0.493 | 491 | 492.2 | [M + H]⁺ | 1 |
| 1-169 | 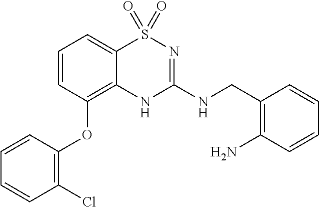 [SMILES: Nc1ccccc1CNC1=NS(=O)(=O)c2cccc(Oc3ccccc3Cl)c2N1] | 0.468 | 428 | 429.2 | [M + H]⁺ | 1 |
| 1-170 | 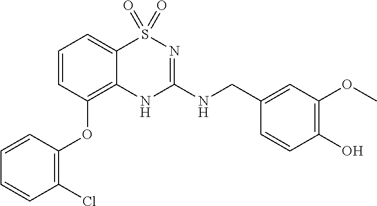 [SMILES: COc1cc(CNC2=NS(=O)(=O)c3cccc(Oc4ccccc4Cl)c3N2)ccc1O] | 0.493 | 459 | 460.2 | [M + H]⁺ | 1 |
| 1-171 | 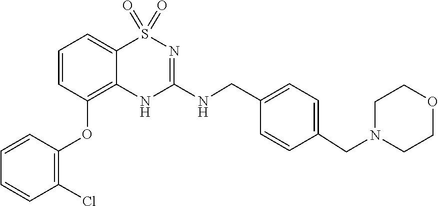 [SMILES: O=S1(=O)N=C(NCc2ccc(CN3CCOCC3)cc2)Nc2c(Oc3ccccc3Cl)cccc21] | 0.427 | 512 | 513.1 | [M + H]⁺ | 1 |
| 1-172 | 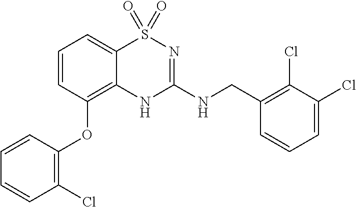 [SMILES: O=S1(=O)N=C(NCc2cccc(Cl)c2Cl)Nc2c(Oc3ccccc3Cl)cccc21] | 0.577 | 481 | 482.1 | [M + H]⁺ | 1 |
| 1-173 | 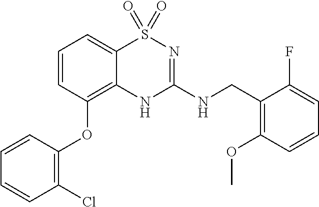 [SMILES: COc1cccc(F)c1CNC1=NS(=O)(=O)c2cccc(Oc3ccccc3Cl)c2N1] | 0.544 | 461 | 462.1 | [M + H]⁺ | 1 |
| 1-174 | 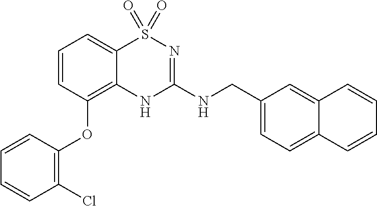 [SMILES: O=S1(=O)N=C(NCc2ccc3ccccc3c2)Nc2c(Oc3ccccc3Cl)cccc21] | 0.577 | 463 | 464 | [M + H]⁺ | 1 |
| 1-175 | 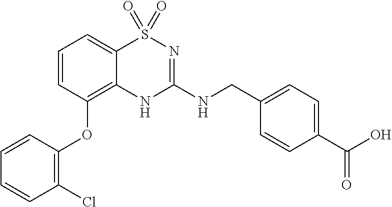 [SMILES: O=C(O)c1ccc(CNC2=NS(=O)(=O)c3cccc(Oc4ccccc4Cl)c3N2)cc1] | 0.485 | 457 | 458 | [M + H]⁺ | 1 |
| 1-176 | 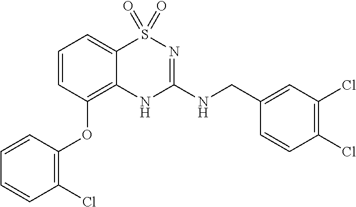 [SMILES: O=S1(=O)N=C(NCc2ccc(Cl)c(Cl)c2)Nc2c(Oc3ccccc3Cl)cccc21] | 0.585 | 481 | 482 | [M + H]⁺ | 1 |
| 1-177 | 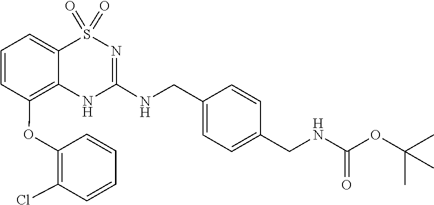 [SMILES: CC(C)(C)OC(=O)NCc1ccc(CNC2=NS(=O)(=O)c3cccc(Oc4ccccc4Cl)c3N2)cc1] | 0.568 | 542 | 443 | [M + H]⁺ | 1 |
| 1-178 | 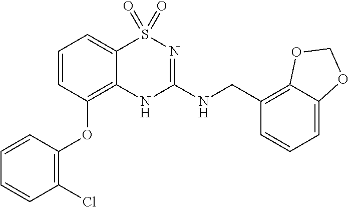 [SMILES: O=S1(=O)N=C(NCc2cccc3c2OCO3)Nc2c(Oc3ccccc3Cl)cccc21] | 0.529 | 457 | 458.1 | [M + H]⁺ | 1 |
| 1-179 | 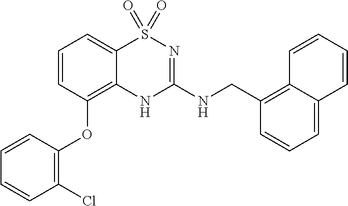 [SMILES: O=S1(=O)N=C(NCc2cccc3ccccc23)Nc2c(Oc3ccccc3Cl)cccc21] | 0.563 | 463 | 464.1 | [M + H]⁺ | 1 |
| 1-180 | 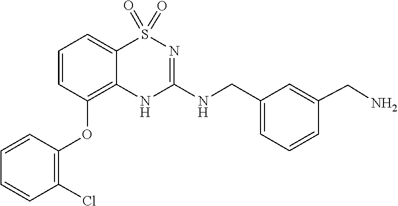 [SMILES: NCc1cccc(CNC2=NS(=O)(=O)c3cccc(Oc4ccccc4Cl)c3N2)c1] | 0.425 | 442 | 443.1 | [M + H]+ | 1 |
| 1-181 | 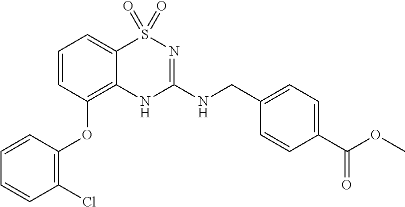 [SMILES: COC(=O)c1ccc(CNC2=NS(=O)(=O)c3cccc(Oc4ccccc4Cl)c3N2)cc1] | 0.529 | 471 | 472.1 | [M + H]⁺ | 1 |
| 1-182 | 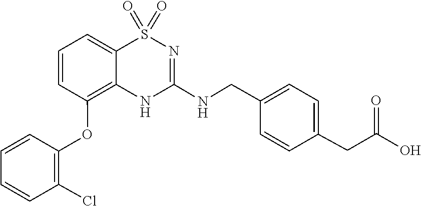 [SMILES: O=C(O)Cc1ccc(CNC2=NS(=O)(=O)c3cccc(Oc4ccccc4Cl)c3N2)cc1] | 0.493 | 471 | 472 | [M + H]⁺ | 1 |
| 1-183 | 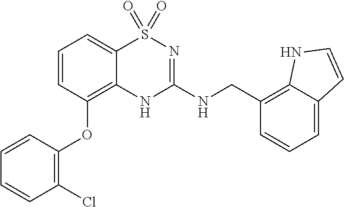 [SMILES: O=S1(=O)N=C(NCc2cccc3cc[nH]c23)Nc2c(Oc3ccccc3Cl)cccc21] | 0.549 | 452 | 453 | [M + H]⁺ | 1 |
| 1-184 | 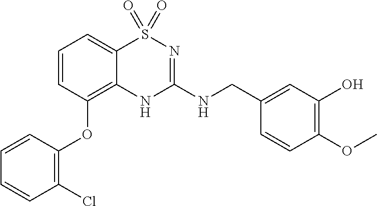 [SMILES: COc1ccc(CNC2=NS(=O)(=O)c3cccc(Oc4ccccc4Cl)c3N2)cc1O] | 0.492 | 459 | 460 | [M + H]+ | 1 |
| 1-185 | 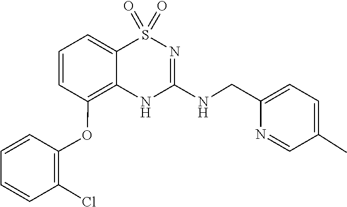 [SMILES: Cc1ccc(CNC2=NS(=O)(=O)c3cccc(Oc4ccccc4Cl)c3N2)nc1] | 0.435 | 428 | 429.1 | [M + H]⁺ | 1 |
| 1-186 | 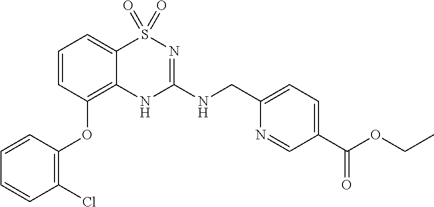 [SMILES: CCOC(=O)c1ccc(CNC2=NS(=O)(=O)c3cccc(Oc4ccccc4Cl)c3N2)nc1] | 0.493 | 486 | 487.2 | [M + H]⁺ | 1 |
| 1-187 | 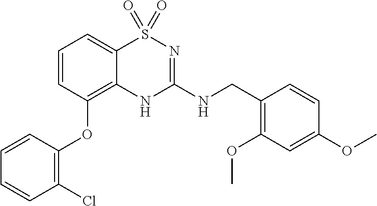 [SMILES: COc1ccc(CNC2=NS(=O)(=O)c3cccc(Oc4ccccc4Cl)c3N2)c(OC)c1] | 0.552 | 473 | 474.1 | [M + H]⁺ | 1 |
| 1-188 | 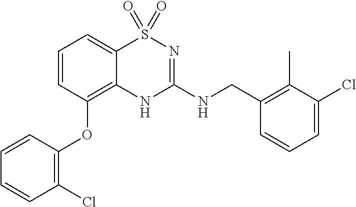 [SMILES: Cc1c(Cl)cccc1CNC1=NS(=O)(=O)c2cccc(Oc3ccccc3Cl)c2N1] | 0.568 | 461 | 462.1 | [M + H]⁺ | 1 |
| 1-189 | 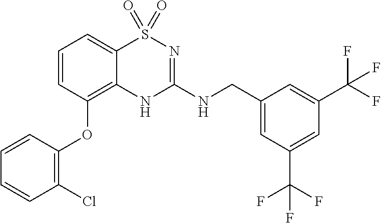 [SMILES: O=S1(=O)N=C(NCc2cc(C(F)(F)F)cc(C(F)(F)F)c2)Nc2c(Oc3ccccc3Cl)cccc21] | 0.593 | 549 | 550.1 | [M + H]⁺ | 1 |
| 1-190 | 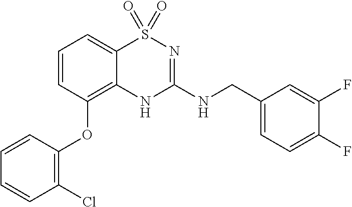 [SMILES: O=S1(=O)N=C(NCc2ccc(F)c(F)c2)Nc2c(Oc3ccccc3Cl)cccc21] | 0.543 | 449 | 450.1 | [M + H]⁺ | 1 |
| 1-191 | 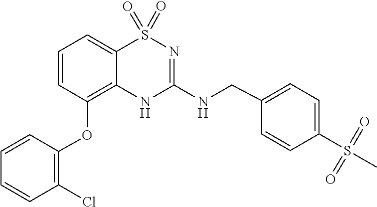 [SMILES: CS(=O)(=O)c1ccc(CNC2=NS(=O)(=O)c3cccc(Oc4ccccc4Cl)c3N2)cc1] | 0.493 | 491 | 492.1 | [M + H]⁺ | 1 |
| 1-192 | 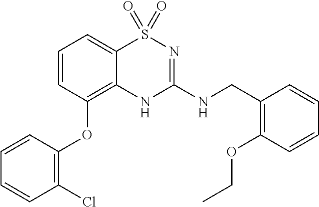 [SMILES: CCOc1ccccc1CNC1=NS(=O)(=O)c2cccc(Oc3ccccc3Cl)c2N1] | 0.568 | 457 | 458.1 | [M + H]⁺ | 1 |
| 1-193 | 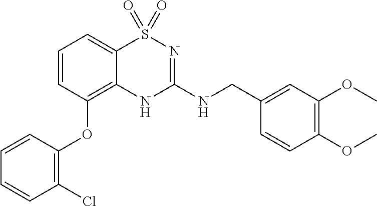 [SMILES: COc1ccc(CNC2=NS(=O)(=O)c3cccc(Oc4ccccc4Cl)c3N2)cc1OC] | 0.518 | 473.0 | 474.1 | [M + H]⁺ | 1 |
| 1-194 | 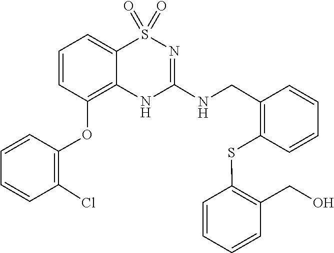 [SMILES: O=S1(=O)N=C(NCc2ccccc2Sc2ccccc2CO)Nc2c(Oc3ccccc3Cl)cccc21] | 0.552 | 551.0 | 552.1 | [M + H]⁺ | 1 |
| 1-195 | 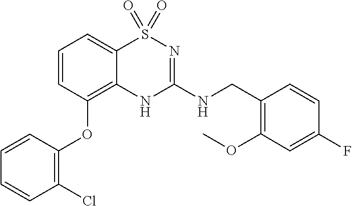 [SMILES: COc1cc(F)ccc1CNC1=NS(=O)(=O)c2cccc(Oc3ccccc3Cl)c2N1] | 0.552 | 461 | 462 | [M + H]⁺ | 1 |
| 1-196 | 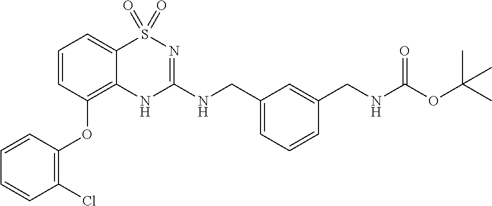 [SMILES: CC(C)(C)OC(=O)NCc1cccc(CNC2=NS(=O)(=O)c3cccc(Oc4ccccc4Cl)c3N2)c1] | 0.558 | 542.1 | 443.1 | [M − Boc + H]⁺ | 1 |
| 1-197 | 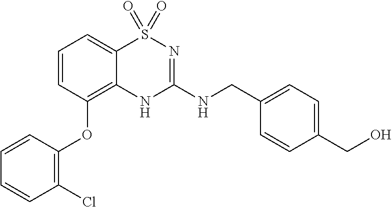 [SMILES: O=S1(=O)N=C(NCc2ccc(CO)cc2)Nc2c(Oc3ccccc3Cl)cccc21] | 0.477 | 443 | 444 | [M + H]⁺ | 1 |
| 1-198 | 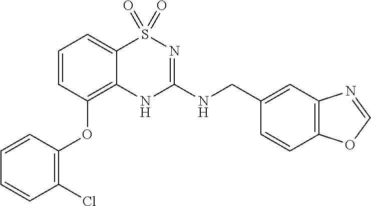 [SMILES: O=S1(=O)N=C(NCc2ccc3ocnc3c2)Nc2c(Oc3ccccc3Cl)cccc21] | 0.493 | 454 | 455.1 | [M + H]⁺ | 1 |
| 1-199 | 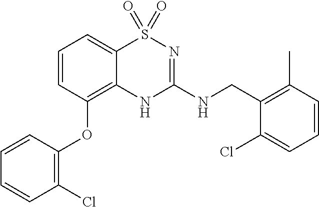 [SMILES: Cc1cccc(Cl)c1CNC1=NS(=O)(=O)c2cccc(Oc3ccccc3Cl)c2N1] | 0.577 | 461 | 462.1 | [M + H]⁺ | 1 |
| 1-200 | 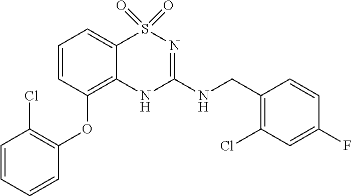 [SMILES: O=S1(=O)N=C(NCc2ccc(F)cc2Cl)Nc2c(Oc3ccccc3Cl)cccc21] | 9.222 | 465 | 466 | [M + H]⁺ | 3 |
| 1-201 | 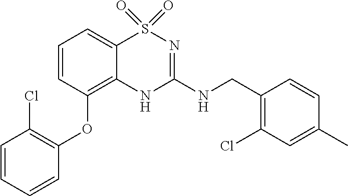 [SMILES: Cc1ccc(CNC2=NS(=O)(=O)c3cccc(Oc4ccccc4Cl)c3N2)c(Cl)c1] | 9.678 | 461 | 462 | [M + H]⁺ | 3 |
| 1-202 | 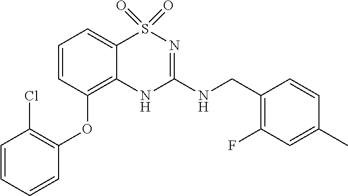 [SMILES: Cc1ccc(CNC2=NS(=O)(=O)c3cccc(Oc4ccccc4Cl)c3N2)c(F)c1] | 9.202 | 445 | 446 | [M + H]⁺ | 3 |
| 1-203 | 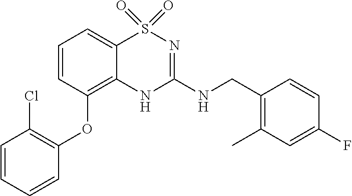 [SMILES: Cc1cc(F)ccc1CNC1=NS(=O)(=O)c2cccc(Oc3ccccc3Cl)c2N1] | 9.103 | 445 | 446 | [M + H]⁺ | 3 |
| 1-204 | 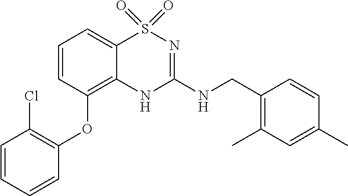 [SMILES: Cc1ccc(CNC2=NS(=O)(=O)c3cccc(Oc4ccccc4Cl)c3N2)c(C)c1] | 9.517 | 441 | 442 | [M + H]⁺ | 3 |
| 1-205 | 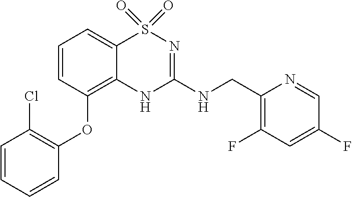 [SMILES: O=S1(=O)N=C(NCc2ncc(F)cc2F)Nc2c(Oc3ccccc3Cl)cccc21] | 8.212 | 450 | 451 | [M + H]⁺ | 3 |
| 1-206 | 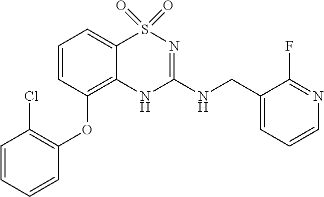 [SMILES: O=S1(=O)N=C(NCc2cccnc2F)Nc2c(Oc3ccccc3Cl)cccc21] | 7.572 | 432 | 433 | [M + H]⁺ | 3 |
| 1-207 | 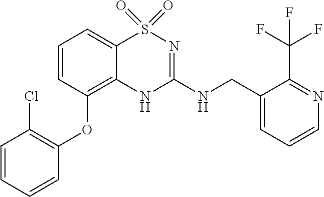 [SMILES: O=S1(=O)N=C(NCc2cccnc2C(F)(F)F)Nc2c(Oc3ccccc3Cl)cccc21] | 8.284 | 482 | 483 | [M + H]⁺ | 3 |
| 1-208 | 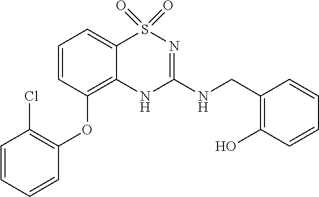 [SMILES: O=S1(=O)N=C(NCc2ccccc2O)Nc2c(Oc3ccccc3Cl)cccc21] | 8.055 | 429 | 430 | [M + H]⁺ | 3 |
| 1-209 | 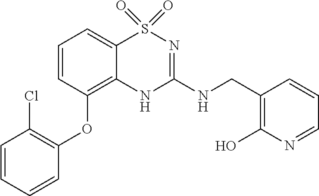 [SMILES: O=S1(=O)N=C(NCc2cccnc2O)Nc2c(Oc3ccccc3Cl)cccc21] | 6.25 | 430 | 431 | [M + H]⁺ | 3 |
| 1-210 | 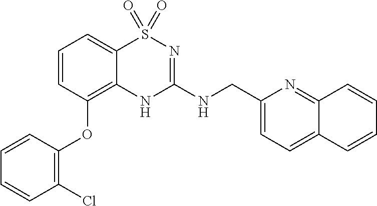 [SMILES: O=S1(=O)N=C(NCc2ccc3ccccc3n2)Nc2c(Oc3ccccc3Cl)cccc21] | 0.485 | 464 | 465.1 | [M + H]⁺ | 1 |
| 1-211 | 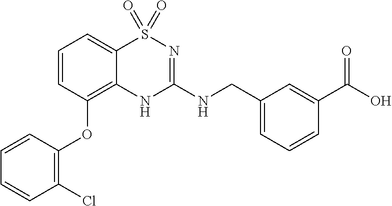 [SMILES: O=C(O)c1cccc(CNC2=NS(=O)(=O)c3cccc(Oc4ccccc4Cl)c3N2)c1] | 0.487 | 457 | 458.1 | [M + H]⁺ | 1 |
| 1-212 | 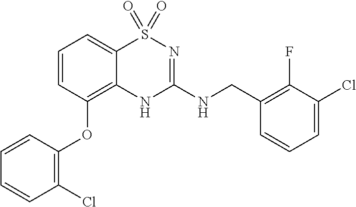 [SMILES: O=S1(=O)N=C(NCc2cccc(Cl)c2F)Nc2c(Oc3ccccc3Cl)cccc21] | 0.560 | 465 | 466.1 | [M + H]⁺ | 1 |
| 1-213 | 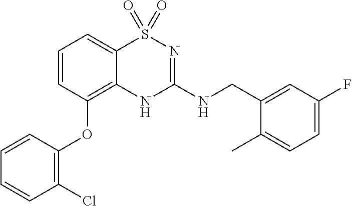 [SMILES: Cc1ccc(F)cc1CNC1=NS(=O)(=O)c2cccc(Oc3ccccc3Cl)c2N1] | 0.552 | 445 | 446.2 | [M + H]⁺ | 1 |
| 1-214 | 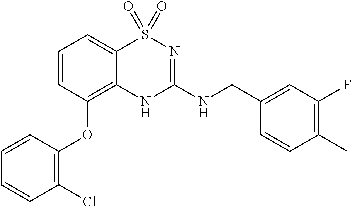 [SMILES: Cc1ccc(CNC2=NS(=O)(=O)c3cccc(Oc4ccccc4Cl)c3N2)cc1F] | 0.560 | 445 | 446.1 | [M + H]⁺ | 1 |
| 1-215 | 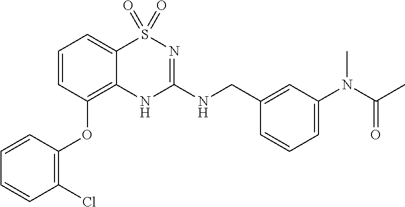 [SMILES: CC(=O)N(C)c1cccc(CNC2=NS(=O)(=O)c3cccc(Oc4ccccc4Cl)c3N2)c1] | 0.483 | 484 | 485.2 | [M + H]⁺ | 1 |
| 1-216 | 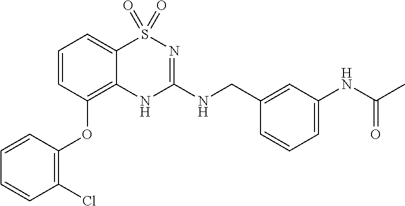 [SMILES: CC(=O)Nc1cccc(CNC2=NS(=O)(=O)c3cccc(Oc4ccccc4Cl)c3N2)c1] | 0.485 | 470 | 471.1 | [M + H]⁺ | 1 |
| 1-217 | 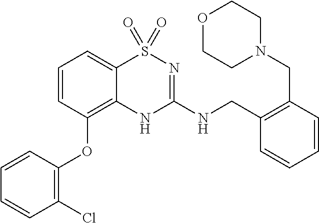 [SMILES: O=S1(=O)N=C(NCc2ccccc2CN2CCOCC2)Nc2c(Oc3ccccc3Cl)cccc21] | 0.444 | 512 | 513.2 | [M + H]⁺ | 1 |
| 1-218 | 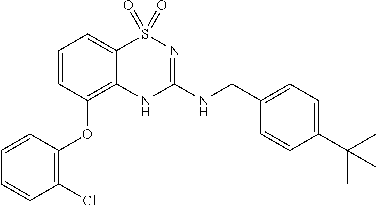 [SMILES: CC(C)(C)c1ccc(CNC2=NS(=O)(=O)c3cccc(Oc4ccccc4Cl)c3N2)cc1] | 0.608 | 469 | 470.2 | [M + H]⁺ | 1 |
| 1-219 | 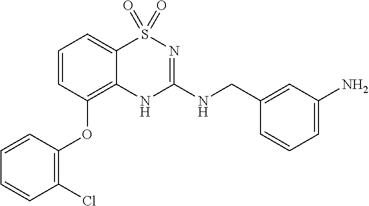 [SMILES: Nc1cccc(CNC2=NS(=O)(=O)c3cccc(Oc4ccccc4Cl)c3N2)c1] | 0.435 | 428 | 429.2 | [M + H]⁺ | 1 |
| 1-220 | 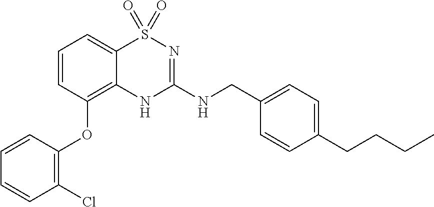 [SMILES: CCCCc1ccc(CNC2=NS(=O)(=O)c3cccc(Oc4ccccc4Cl)c3N2)cc1] | 0.615 | 469 | 470.3 | [M + H]⁺ | 1 |
| 1-221 | 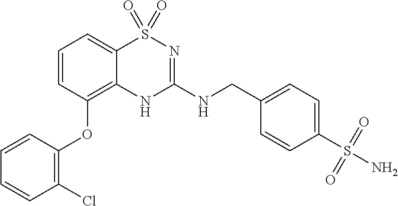 [SMILES: NS(=O)(=O)c1ccc(CNC2=NS(=O)(=O)c3cccc(Oc4ccccc4Cl)c3N2)cc1] | 0.479 | 492 | 493.1 | [M + H]⁺ | 1 |
| 1-222 | 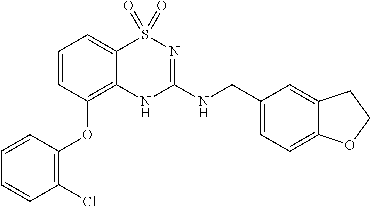 [SMILES: O=S1(=O)N=C(NCc2ccc3c(c2)CCO3)Nc2c(Oc3ccccc3Cl)cccc21] | 0.532 | 455 | 456.2 | [M + H]⁺ | 1 |
| 1-223 | 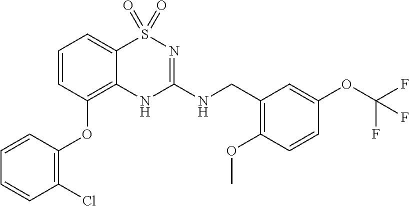 [SMILES: COc1ccc(OC(F)(F)F)cc1CNC1=NS(=O)(=O)c2cccc(Oc3ccccc3Cl)c2N1] | 0.578 | 527 | 528.2 | [M + H]⁺ | 1 |
| 1-224 | 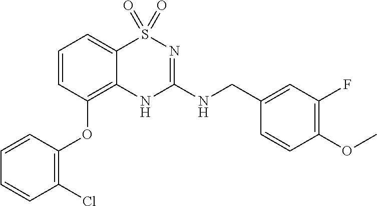 [SMILES: COc1ccc(CNC2=NS(=O)(=O)c3cccc(Oc4ccccc4Cl)c3N2)cc1F] | 0.537 | 461 | 462.2 | [M + H]⁺ | 1 |
| 1-225 | 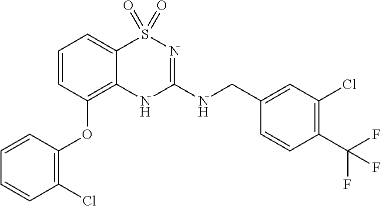 [SMILES: O=S1(=O)N=C(NCc2ccc(C(F)(F)F)c(Cl)c2)Nc2c(Oc3ccccc3Cl)cccc21] | 0.585 | 515 | 516.1 | [M + H]⁺ | 1 |
| 1-226 | 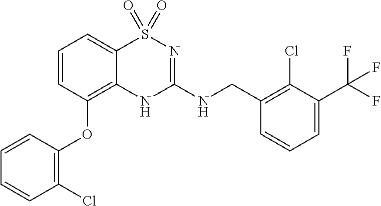 [SMILES: O=S1(=O)N=C(NCc2cccc(C(F)(F)F)c2Cl)Nc2c(Oc3ccccc3Cl)cccc21] | 0.575 | 515 | 516.1 | [M + H]⁺ | 1 |
| 1-227 | 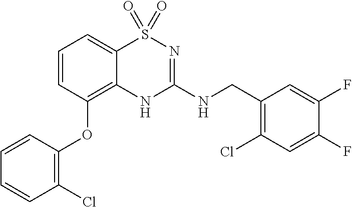 [SMILES: O=S1(=O)N=C(NCc2cc(F)c(F)cc2Cl)Nc2c(Oc3ccccc3Cl)cccc21] | 0.565 | 483 | 484.1 | [M + H]⁺ | 1 |
| 1-228 | 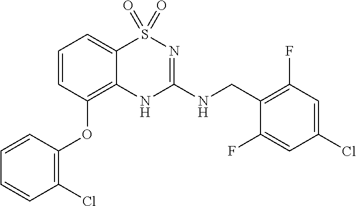 [SMILES: O=S1(=O)N=C(NCc2c(F)cc(Cl)cc2F)Nc2c(Oc3ccccc3Cl)cccc21] | 0.567 | 483 | 484.1 | [M + H]⁺ | 1 |
| 1-229 | 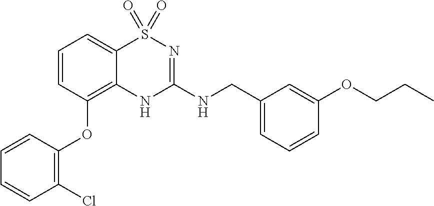 [SMILES: CCCOc1cccc(CNC2=NS(=O)(=O)c3cccc(Oc4ccccc4Cl)c3N2)c1] | 0.585 | 471 | 472.2 | [M + H]⁺ | 1 |
| 1-230 | 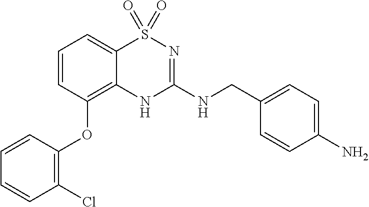 [SMILES: Nc1ccc(CNC2=NS(=O)(=O)c3cccc(Oc4ccccc4Cl)c3N2)cc1] | 0.430 | 428 | 429.1 | [M + H]⁺ | 1 |
| 1-231 | 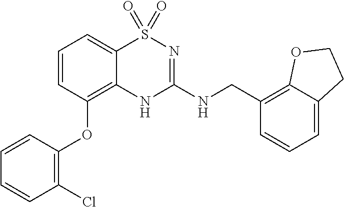 [SMILES: O=S1(=O)N=C(NCc2cccc3c2OCC3)Nc2c(Oc3ccccc3Cl)cccc21] | 0.537 | 455 | 456.2 | [M + H]⁺ | 1 |
| 1-232 | 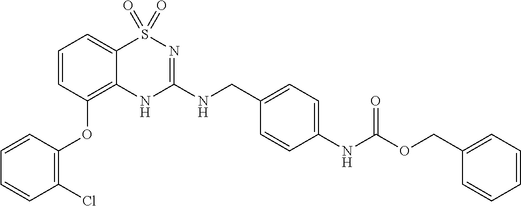 [SMILES: O=C(Nc1ccc(CNC2=NS(=O)(=O)c3cccc(Oc4ccccc4Cl)c3N2)cc1)OCc1ccccc1] | 0.570 | 562 | 563.3 | [M + H]⁺ | 1 |
| 1-233 | 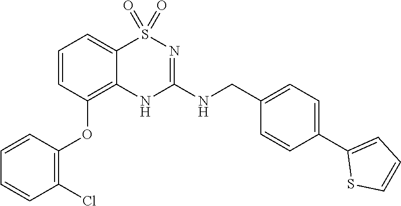 [SMILES: O=S1(=O)N=C(NCc2ccc(-c3cccs3)cc2)Nc2c(Oc3ccccc3Cl)cccc21] | 0.589 | 495 | 496.1 | [M + H]⁺ | 1 |
| 1-234 | 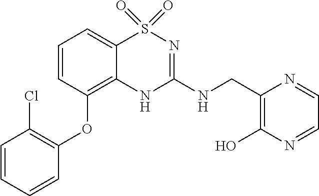 [SMILES: O=S1(=O)N=C(NCc2nccnc2O)Nc2c(Oc3ccccc3Cl)cccc21] | 6.221 | 431 | 432 | [M + H]⁺ | 3 |
| 1-235 | 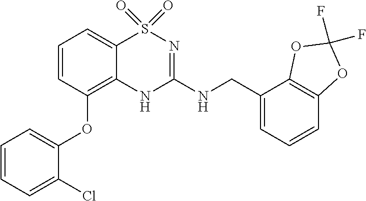 [SMILES: O=S1(=O)N=C(NCc2cccc3c2OC(F)(F)O3)Nc2c(Oc3ccccc3Cl)cccc21] | 9.336 | 493 | 494 | [M + H]⁺ | 3 |
| 1-236 | 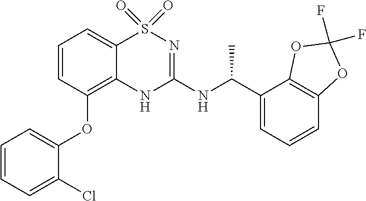 [SMILES: C[C@@H](NC1=NS(=O)(=O)c2cccc(Oc3ccccc3Cl)c2N1)c1cccc2c1OC(F)(F)O2] | 9.707 | 507 | 508 | [M + H]⁺ | 3 |
| 1-237 | 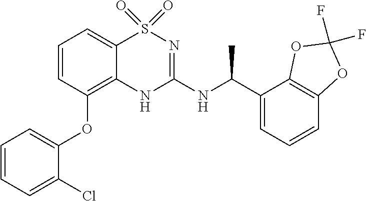 [SMILES: C[C@H](NC1=NS(=O)(=O)c2cccc(Oc3ccccc3Cl)c2N1)c1cccc2c1OC(F)(F)O2] | 9.695 | 507 | 508 | [M + H]⁺ | 3 |
| 1-238 | 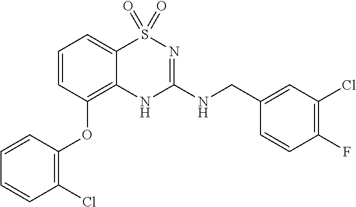 [SMILES: O=S1(=O)N=C(NCc2ccc(F)c(Cl)c2)Nc2c(Oc3ccccc3Cl)cccc21] | 0.558 | 465 | 466.1 | [M + H]⁺ | 1 |
| 1-239 | 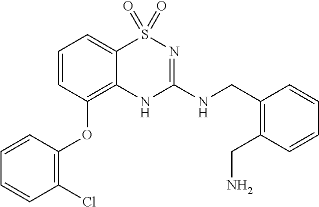 [SMILES: NCc1ccccc1CNC1=NS(=O)(=O)c2cccc(Oc3ccccc3Cl)c2N1] | 0.424 | 442 | 443.1 | [M + H]⁺ | 1 |
| 1-240 | 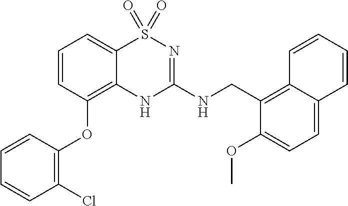 [SMILES: COc1ccc2ccccc2c1CNC1=NS(=O)(=O)c2cccc(Oc3ccccc3Cl)c2N1] | 0.581 | 493 | 494.2 | [M + H]⁺ | 11 |
| 1-241 | 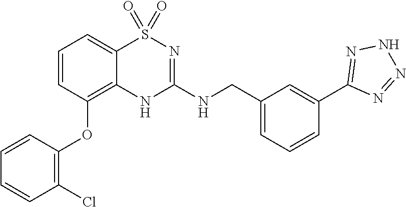 [SMILES: O=S1(=O)N=C(NCc2cccc(-c3nn[nH]n3)c2)Nc2c(Oc3ccccc3Cl)cccc21] | 0.481 | 481 | 482.3 | [M + H]⁺ | 1 |
| 1-242 | 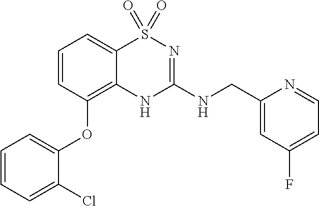 [SMILES: O=S1(=O)N=C(NCc2cc(F)ccn2)Nc2c(Oc3ccccc3Cl)cccc21] | 7.366 | 432 | 433 | [M + H]⁺ | 3 |
| 1-243 | 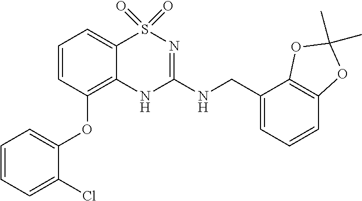 [SMILES: CC1(C)Oc2cccc(CNC3=NS(=O)(=O)c4cccc(Oc5ccccc5Cl)c4N3)c2O1] | 9.332 | 485 | 486 | [M + H]⁺ | 3 |
| 1-244 | 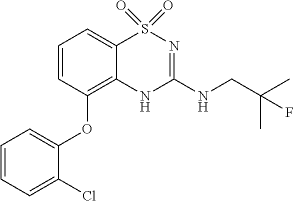 [SMILES: CC(C)(F)CNC1=NS(=O)(=O)c2cccc(Oc3ccccc3Cl)c2N1] | 8.135 | 397 | 398 | [M + H]⁺ | 3 |
| 1-245 | 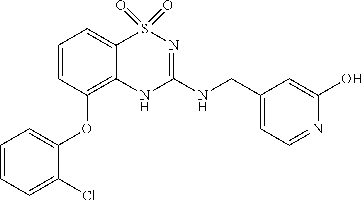 [SMILES: O=S1(=O)N=C(NCc2ccnc(O)c2)Nc2c(Oc3ccccc3Cl)cccc21] | 5.87 | 430 | 431 | [M + H]⁺ | 3 |
| 1-246 | 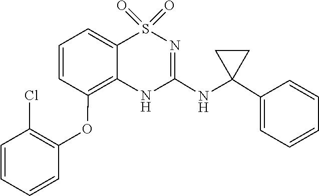 [SMILES: O=S1(=O)N=C(NC2(c3ccccc3)CC2)Nc2c(Oc3ccccc3Cl)cccc21] | 9.168 | 439 | 440 | [M + H]⁺ | 3 |
| 1-247 | 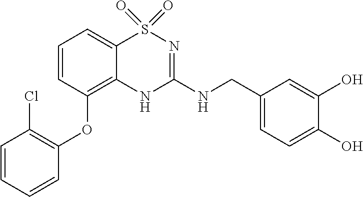 [SMILES: O=S1(=O)N=C(NCc2ccc(O)c(O)c2)Nc2c(Oc3ccccc3Cl)cccc21] | 9.168 | 445 | 446 | [M + H]⁺ | 3 |
| 1-248 | 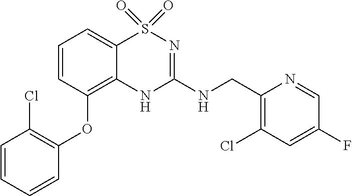 [SMILES: O=S1(=O)N=C(NCc2ncc(F)cc2Cl)Nc2c(Oc3ccccc3Cl)cccc21] | 8.801 | 466 | 467 | [M + H]⁺ | 3 |
| 1-249 | 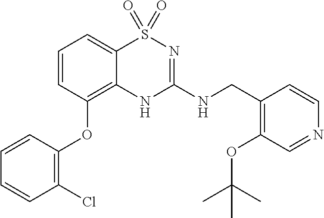 [SMILES: CC(C)(C)Oc1cnccc1CNC1=NS(=O)(=O)c2cccc(Oc3ccccc3Cl)c2N1] | 6.244 | 486 | 487 | [M + H]⁺ | 3 |
| 1-250 | 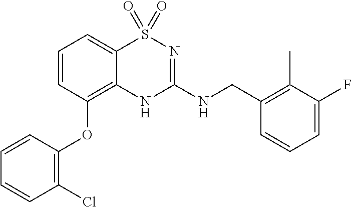 [SMILES: Cc1c(F)cccc1CNC1=NS(=O)(=O)c2cccc(Oc3ccccc3Cl)c2N1] | 0.553 | 445 | 446.3 | [M + H]⁺ | 1 |
| 1-251 | 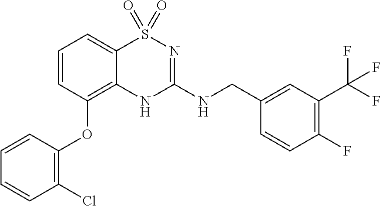 [SMILES: O=S1(=O)N=C(NCc2ccc(F)c(C(F)(F)F)c2)Nc2c(Oc3ccccc3Cl)cccc21] | 0.571 | 499 | 500.2 | [M + H]⁺ | 1 |
| 1-252 | 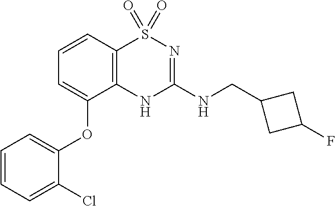 [SMILES: O=S1(=O)N=C(NCC2CC(F)C2)Nc2c(Oc3ccccc3Cl)cccc21] | 0.516 | 409 | 410.3 | [M + H]⁺ | 1 |
| 1-253 | 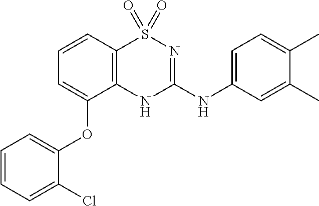 [SMILES: Cc1ccc(NC2=NS(=O)(=O)c3cccc(Oc4ccccc4Cl)c3N2)cc1C] | 0.558 | 427 | 428.3 | [M + H]⁺ | 1 |
| 1-254 | 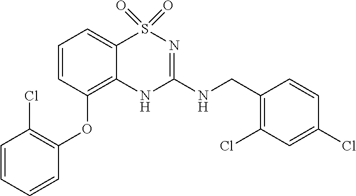 [SMILES: O=S1(=O)N=C(NCc2ccc(Cl)cc2Cl)Nc2c(Oc3ccccc3Cl)cccc21] | 9.882 | 481 | 481 | [M + H]⁺ | 3 |
| 1-255 | 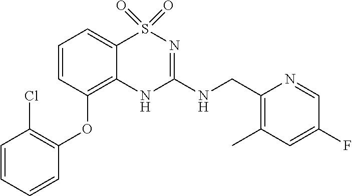 [SMILES: Cc1cc(F)cnc1CNC1=NS(=O)(=O)c2cccc(Oc3ccccc3Cl)c2N1] | 8.355 | 446 | 447 | [M + H]⁺ | 3 |
| 1-256 | 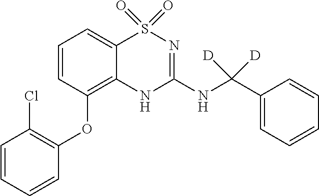 [SMILES: [2H]C([2H])(NC1=NS(=O)(=O)c2cccc(Oc3ccccc3Cl)c2N1)c1ccccc1] | 8.764 | 415 | 416 | [M + H]⁺ | 3 |
| 1-257 | 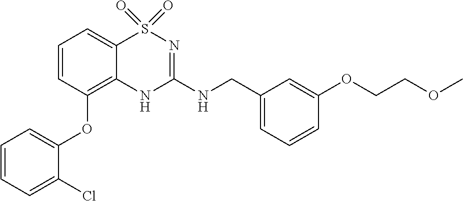 [SMILES: COCCOc1cccc(CNC2=NS(=O)(=O)c3cccc(Oc4ccccc4Cl)c3N2)c1] | 8.51 | 487 | 488 | [M + H]⁺ | 3 |
| 1-258 | 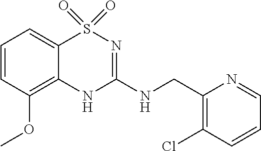 [SMILES: COc1cccc2c1NC(NCc1ncccc1Cl)=NS2(=O)=O] | 5.837 | 352 | 353 | [M + H]⁺ | 3 |
| 1-259 | 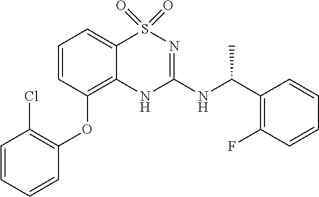 [SMILES: C[C@@H](NC1=NS(=O)(=O)c2cccc(Oc3ccccc3Cl)c2N1)c1ccccc1F] | 8.922 | 445 | 446 | [M + H]⁺ | 3 |
| 1-260 | 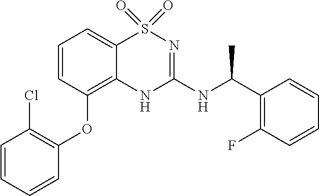 [SMILES: C[C@H](NC1=NS(=O)(=O)c2cccc(Oc3ccccc3Cl)c2N1)c1ccccc1F] | 9.149 | 445 | 446 | [M + H]⁺ | 3 |
| 1-261 | 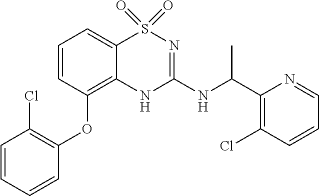 [SMILES: CC(NC1=NS(=O)(=O)c2cccc(Oc3ccccc3Cl)c2N1)c1ncccc1Cl] | 9.159 | 462 | 463 | [M + H]⁺ | 3 |
| 1-262 | 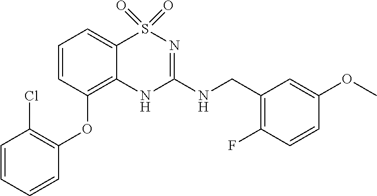 [SMILES: COc1ccc(F)c(CNC2=NS(=O)(=O)c3cccc(Oc4ccccc4Cl)c3N2)c1] | 8.866 | 461 | 462 | [M + H]⁺ | 3 |
| 1-263 | 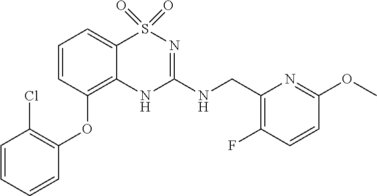 [SMILES: COc1ccc(F)c(CNC2=NS(=O)(=O)c3cccc(Oc4ccccc4Cl)c3N2)n1] | 8.766 | 462 | 463 | [M + H]⁺ | 3 |
| 1-264 | 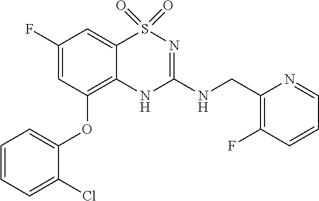 [SMILES: O=S1(=O)N=C(NCc2ncccc2F)Nc2c(Oc3ccccc3Cl)cc(F)cc21] | 8.256 | 450 | 451 | [M + H]⁺ | 3 |
| 1-265 | 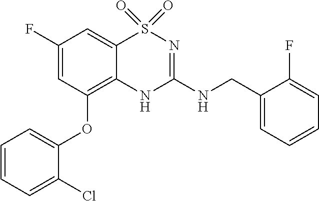 [SMILES: O=S1(=O)N=C(NCc2ccccc2F)Nc2c(Oc3ccccc3Cl)cc(F)cc21] | 9.07 | 449 | 450 | [M + H]⁺ | 3 |
| 1-266 | 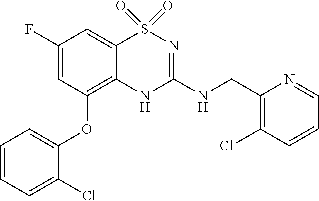 [SMILES: O=S1(=O)N=C(NCc2ncccc2Cl)Nc2c(Oc3ccccc3Cl)cc(F)cc21] | 8.91 | 466 | 467 | [M + H]⁺ | 3 |
| 1-267 | 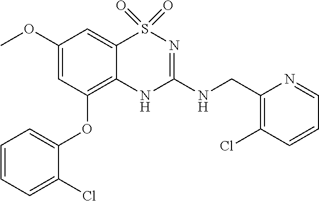 [SMILES: COc1cc(Oc2ccccc2Cl)c2c(c1)S(=O)(=O)N=C(NCc1ncccc1Cl)N2] | 8.676 | 478 | 479 | [M + H]⁺ | 3 |
| 1-268 | 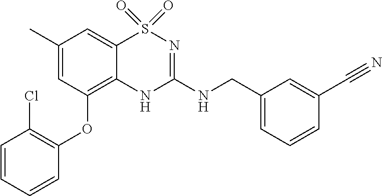 [SMILES: Cc1cc(Oc2ccccc2Cl)c2c(c1)S(=O)(=O)N=C(NCc1cccc(C#N)c1)N2] | 8.731 | 452 | 453 | [M + H]⁺ | 3 |
| 1-269 | 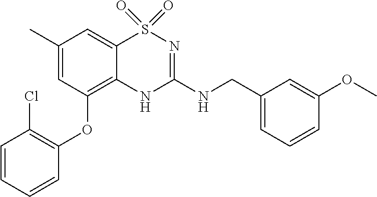 [SMILES: COc1cccc(CNC2=NS(=O)(=O)c3cc(C)cc(Oc4ccccc4Cl)c3N2)c1] | 9.135 | 457 | 458 | [M + H]⁺ | 3 |
| 1-270 | 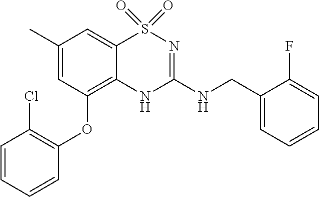 [SMILES: Cc1cc(Oc2ccccc2Cl)c2c(c1)S(=O)(=O)N=C(NCc1ccccc1F)N2] | 9.226 | 445 | 446 | [M + H]⁺ | 3 |
| 1-271 | 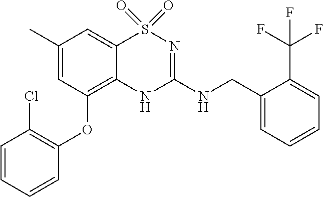 [SMILES: Cc1cc(Oc2ccccc2Cl)c2c(c1)S(=O)(=O)N=C(NCc1ccccc1C(F)(F)F)N2] | 9.765 | 495 | 496 | [M + H]⁺ | 3 |
| 1-272 | 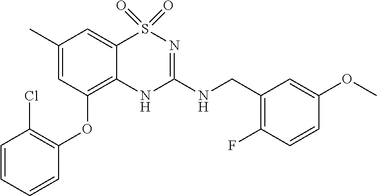 [SMILES: COc1ccc(F)c(CNC2=NS(=O)(=O)c3cc(C)cc(Oc4ccccc4Cl)c3N2)c1] | 9.221 | 475 | 476 | [M + H]⁺ | 3 |
| 1-273 | 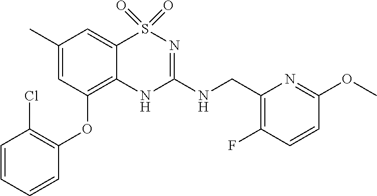 [SMILES: COc1ccc(F)c(CNC2=NS(=O)(=O)c3cc(C)cc(Oc4ccccc4Cl)c3N2)n1] | 9.228 | 476 | 477 | [M + H]⁺ | 3 |
| 1-274 | 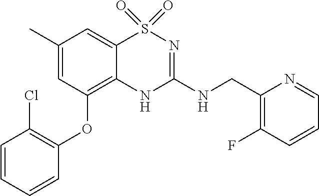 [SMILES: Cc1cc(Oc2ccccc2Cl)c2c(c1)S(=O)(=O)N=C(NCc1ncccc1F)N2] | 8.349 | 446 | 447 | [M + H]⁺ | 3 |
| 1-275 | 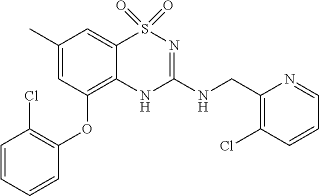 [SMILES: Cc1cc(Oc2ccccc2Cl)c2c(c1)S(=O)(=O)N=C(NCc1ncccc1Cl)N2] | 8.89 | 462 | 463 | [M + H]⁺ | 3 |
| 1-276 | 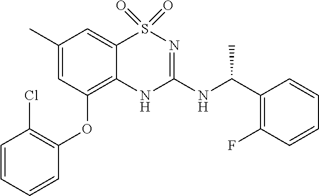 [SMILES: Cc1cc(Oc2ccccc2Cl)c2c(c1)S(=O)(=O)N=C(N[C@H](C)c1ccccc1F)N2] | 9.534 | 459 | 460 | [M + H]⁺ | 3 |
| 1-277 | 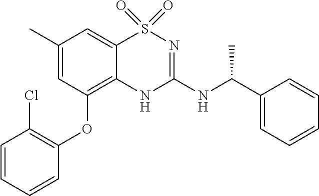 [SMILES: Cc1cc(Oc2ccccc2Cl)c2c(c1)S(=O)(=O)N=C(N[C@H](C)c1ccccc1)N2] | 9.619 | 441 | 441 | [M] | 3 |
| 1-278 | 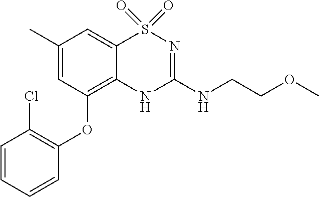 [SMILES: COCCNC1=NS(=O)(=O)c2cc(C)cc(Oc3ccccc3Cl)c2N1] | 7.769 | 395 | 396 | [M + H]⁺ | 3 |
| 1-279 | 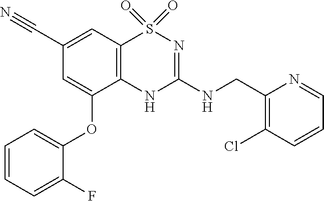 [SMILES: N#Cc1cc(Oc2ccccc2F)c2c(c1)S(=O)(=O)N=C(NCc1ncccc1Cl)N2] | 0.511 | 457 | 458 | [M + H]⁺ | 1 |
| 1-280 | 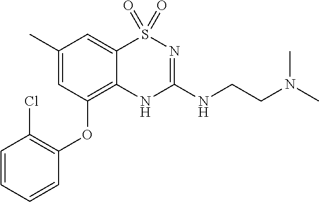 [SMILES: Cc1cc(Oc2ccccc2Cl)c2c(c1)S(=O)(=O)N=C(NCCN(C)C)N2] | 5.172 | 408.1 | 409.2 | [M + H]⁺ | 3 |

### Example 2

### Synthesis of Representative Compound 2-1

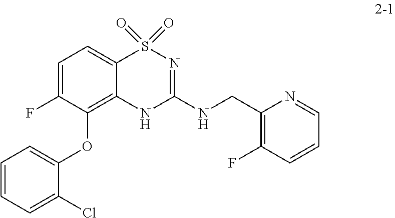 [SMILES: O=S1(=O)N=C(NCc2ncccc2F)Nc2c1ccc(F)c2Oc1ccccc1Cl]

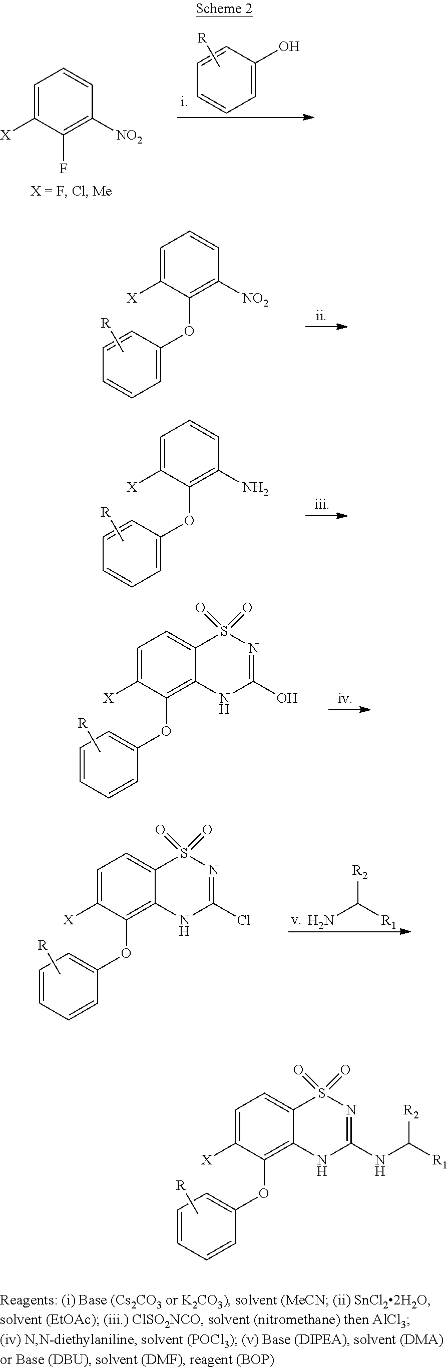

### Step 2-1. Synthesis of 2-(2-chlorophenoxy)-1-fluoro-3-nitrobenzene (INT 2A)

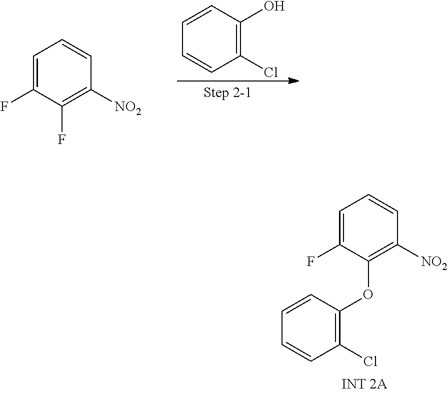 [SMILES: O=[N+]([O-])c1cccc(F)c1F ; Oc1ccccc1Cl ; O=[N+]([O-])c1cccc(F)c1Oc1ccccc1Cl]

To a stirring solution of 1,2-difluoro-3-nitro-benzene (3.0 g, 18.86 mmol, 8.19 mL, 1.2 eq) and 2-chlorophenol (2.02 g, 15.71 mmol, 1.60 mL, 1 eq) in MeCN (200 mL) was added Cs₂CO₃ (10.2 g, 31.4 mmol, 2 eq). The reaction mixture was heated and stirred at 80° C. for 14 h. The reaction mixture was cooled to room temperature, filtered through Celite, and concentrated in vacuo to afford crude material. The crude material was purified by silica gel chromatography (0-3% EtOAc/petroleum ether) to afford 2-(2-chlorophenoxy)-1-fluoro-3-nitro-benzene INT 2A (3.1 g, 11.6 mmol, 74% yield). ¹H NMR (400 MHz, DMSO-d6) δ=8.04 (td, J=1.5, 8.4 Hz, 1H), 7.88 (ddd, J=1.5, 8.6, 10.3 Hz, 1H), 7.65-7.58 (m, 2H), 7.31-7.24 (m, 1H), 7.16 (dt, J=1.4, 7.7 Hz, 1H), 6.87 (d, J=8.1 Hz, 1H).

### Step 2-2. Synthesis of 2-(2-chlorophenoxy)-3-fluoroaniline (INT 2B)

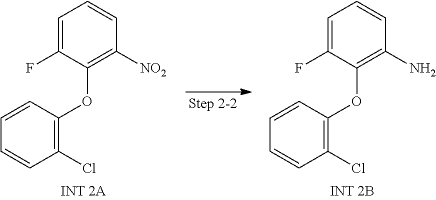 [SMILES: O=[N+]([O-])c1cccc(F)c1Oc1ccccc1Cl ; Nc1cccc(F)c1Oc1ccccc1Cl]

To a solution of 2-(2-chlorophenoxy)-1-fluoro-3-nitro-benzene INT 2A (3.1 g, 11.6 mmol, 1 eq) in EA (25 mL), was added SnCl₂·2H₂O (15.7 g, 69.5 mmol, 6 eq). The mixture was heated and stirred at 50° C. for 16 h. The reaction mixture was cooled to room temperature, filtered through a pad of Celite, and concentrated in vacuo to afford crude material. The crude material was purified by silica gel chromatography (0-5% EtOAc/petroleum ether) to afford 2-(2-chlorophenoxy)-3-fluoroaniline INT 2B (2.5 g, 10.2 mmol, 88% yield). LCMS (m/z) calculated for C₁₂H₉ClFNO: 237.0; found 238.0 [M+H]+, tR=0.543 min (Purity Method 1).

### Step 2-3. Synthesis of 5-(2-chlorophenoxy)-6-fluoro-3-hydroxy-4H-benzo[e][1,2,4]thiadiazine 1,1-dioxide (INT 2C)

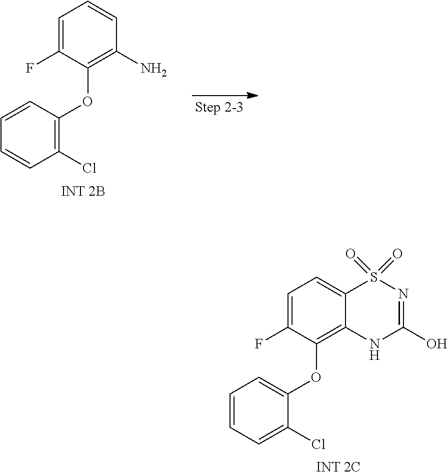 [SMILES: Nc1cccc(F)c1Oc1ccccc1Cl ; O=S1(=O)N=C(O)Nc2c1ccc(F)c2Oc1ccccc1Cl]

To a solution of N-(oxomethylene)sulfamoyl chloride INT 2B (2.28 g, 16.1 mmol, 1.40 mL, 1.5 eq) in nitrobenzene (30 mL) at 0° C., was added 2-(2-chlorophenoxy)-3-fluoro-aniline (2.5 g, 10.5 mmol, 3.2 mL, 1 eq). The mixture was stirred at 0° C. for 0.5 h. Then, AlCl₃ (2.10 g, 15.8 mmol, 862 μL, 1.5 eq) was added. The mixture was heated and stirred at 120° C. for 1.5h. The reaction solution was cooled to room temperature and then diluted with aqueous saturated NH₄Cl solution (100 mL). The aqueous phase was extracted with ethyl acetate (3×100 mL). The combined organic layers were washed with brine (80 mL), dried over anhydrous sodium sulfate, filtered, and concentrated in vacuo to give crude material. The crude material was diluted with petroleum ether (300 mL) and then stirred for 30 min. The brown oil eventually formed a brown precipitate that was collected by vacuum filtration. The filter cake was washed with petroleum ether (200 mL) and then dried under vacuum to afford 5-(2-chlorophenoxy)-6-fluoro-3-hydroxy-4H-benzo[e][1,2,4]thiadiazine 1,1-dioxide (INT 2C) (3.0 g, 5.16 mmol, 49% yield) that was used without further purification.

### Step 2-4. Synthesis of 3-chloro-5-(2-chlorophenoxy)-6-fluoro-4H-benzo[e][1,2,4]thiadiazine 1,1-dioxide (INT 2D)

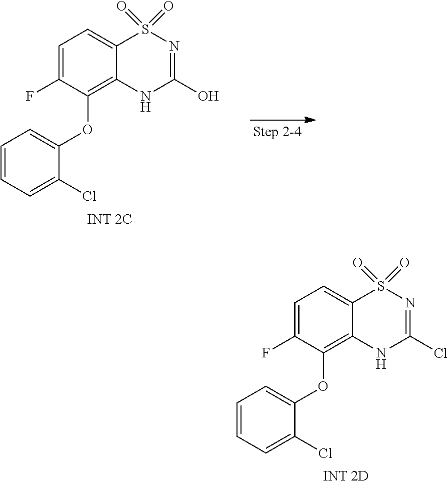 [SMILES: O=S1(=O)N=C(O)Nc2c1ccc(F)c2Oc1ccccc1Cl ; O=S1(=O)N=C(Cl)Nc2c1ccc(F)c2Oc1ccccc1Cl]

To a solution of 5-(2-chlorophenoxy)-6-fluoro-3-hydroxy-4H-benzo[e][1,2,4]thiadiazine 1,1-dioxide INT 2C (1.5 g, 4.38 mmol, 1 eq) in POCl₃ (10 mL), was added N,N-diethylaniline (980 mg, 6.56 mmol, 1.05 mL, 1.5 eq). The mixture was heated and stirred at 120° C. for 16 h. The reaction mixture was cooled to room temperature and poured into water (150 mL) portionwise while the temperature was kept below 35° C. The obtained mixture was stirred for 1 h. The mixture was then extracted with EtOAc (3×100 mL). The combined organics were washed with brine (2×60 mL), dried over Na₂SO₄, filtered, and concentrated in vacuo to give a residue. The aqueous mixture was adjusted to pH=7 by KOH and then discarded. The crude residue was purified by silica gel chromatography (0300% EtOAc/petroleum ether) to afford 3-chloro-5-(2-chlorophenoxy)-6-fluoro-4H-benzo[e][1,2,4]thiadiazine 1,1-dioxide INT 2D (630 mg, 1.73 mmol, 39% yield). LCMS (m/z) calculated for C₁₃H₇Cl₂FN₂O_{3S}: 360.0; found 361.0 [M+H]+, tR=0.527 min (Purity Method 1).

### Step 2-5. Synthesis of 5-(2-chlorophenoxy)-6-fluoro-3-(((3-fluoropyridin-2-yl)methyl)amino)-4H-benzo[e][1,2,4]thiadiazine 1,1-dioxide (Compound 2-1)

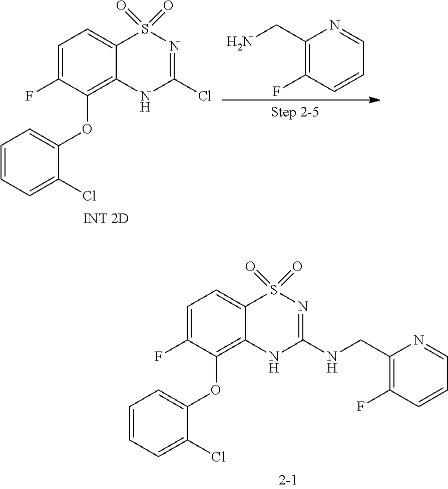 [SMILES: O=S1(=O)N=C(Cl)Nc2c1ccc(F)c2Oc1ccccc1Cl ; NCc1ncccc1F ; O=S1(=O)N=C(NCc2ncccc2F)Nc2c1ccc(F)c2Oc1ccccc1Cl]

To a solution of (3-fluoro-2-pyridyl)methanamine (124 mg, 623 μmol, 1.5 eq, 2HCl) and DIPEA (161 mg, 1.25 mmol, 217 μL, 3 eq) in DMA (2 mL), was added 3-chloro-5-(2-chlorophenoxy)-6-fluoro-4H-benzo[e][1,2,4]thiadiazine 1,1-dioxide INT 2D (150 mg, 415 μmol, 1 eq). The reaction mixture was heated and stirred at 120° C. for 14 h. After returning to room temperature, the crude mixture was filtered through filter paper and concentrated in vacuo. The crude residue was purified by preparative HPLC (column: Phenomenex Luna C18 150×25 mm×10 μm; 38%-68% MeCN and 0.1% formic acid in water over 10 min) and lyophilized to afford 5-(2-chlorophenoxy)-6-fluoro-3-(((3-fluoropyridin-2-yl)methyl)amino)-4H-benzo[e][1,2,4]thiadiazine 1,1-dioxide 2-1 (74 mg, 156 μmol, 38% yield). LCMS (m/z) calculated for C₁₉H₁₃ClF₂N₄O₃S: 450.0; found 451.0 [M+H]+, tR=0.516 min (Purity Method 1). ¹H NMR (400 MHz, DMSO-d6) δ=10.80 (br s, 1H), 8.43 (br d, J=4.0 Hz, 1H), 8.07 (br s, 1H), 7.82-7.74 (m, 1H), 7.70 (dd, J=5.2, 8.8 Hz, 1H), 7.63 (dd, J=1.5, 7.9 Hz, 1H), 7.48 (td, J=4.4, 8.5 Hz, 1H), 7.34-7.25 (m, 2H), 7.24-7.16 (m, 1H), 6.93 (d, J=8.0 Hz, 1H), 4.69 (br d, J=3.8 Hz, 2H).

### Synthesis of Scheme 2 Compounds

Alternatively for step 2-1, the following compounds were made using K₂CO₃ as base instead of Cs₂CO₃: 2-7.

Alternatively for step 2-5, the following compounds were made using THE as solvent instead of DMA at a reaction temperature of 60° C.: 2-3, 2-9, 2-13.

Alternatively for step 2-5, the following compounds were made using 1.3 Eq of BOP reagent with 3.4 Eq of DBU as base and DMF as solvent at 80° C. for 15 h: 2-7.

Alternatively for step 2-5, the following compounds were made using 1.3 Eq of BOP reagent with 3.4 Eq of DBU as base and MeCN as solvent at 80° C. for 15 h: 2-12.

The compounds listed in Table 3 were made using the procedures of Scheme 2.

TABLE 3

| | | Purity | Exact | | | |
|---|---|---|---|---|---|---|
| Cpd | | RT | mass | observed | | Purity |
| No. | Structure | (min) | (g/mol) | m/z | Ion | Method |
| 2-1 | 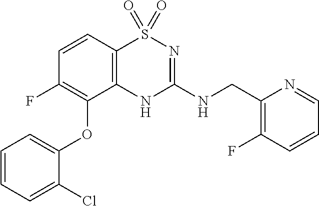 [SMILES: O=S1(=O)N=C(NCc2ncccc2F)Nc2c1ccc(F)c2Oc1ccccc1Cl] | 0.516 | 450.0 | 451.1 | [M + H]⁺ | 1 |
| 2-2 | 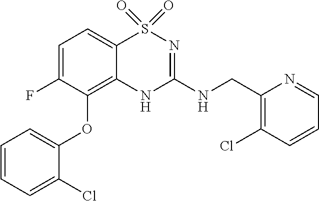 [SMILES: O=S1(=O)N=C(NCc2ncccc2Cl)Nc2c1ccc(F)c2Oc1ccccc1Cl] | 0.532 | 466.0 | 467.2 | [M + H]⁺ | 1 |
| 2-3 | 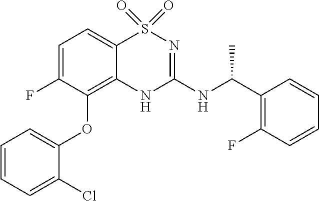 [SMILES: C[C@@H](NC1=NS(=O)(=O)c2ccc(F)c(Oc3ccccc3Cl)c2N1)c1ccccc1F] | 0.551 | 463.1 | 464.3 | [M + H]⁺ | 1 |
| 2-4 | 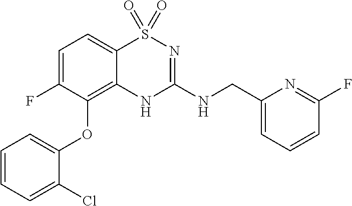 [SMILES: O=S1(=O)N=C(NCc2cccc(F)n2)Nc2c1ccc(F)c2Oc1ccccc1Cl] | 0.515 | 450.0 | 451.0 | [M + H]⁺ | 1 |
| 2-5 | 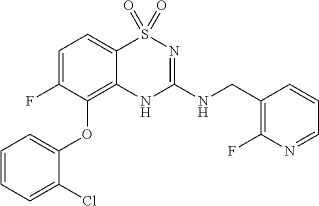 [SMILES: O=S1(=O)N=C(NCc2cccnc2F)Nc2c1ccc(F)c2Oc1ccccc1Cl] | 0.504 | 450.0 | 451.1 | [M + H]⁺ | 1 |
| 2-6 | 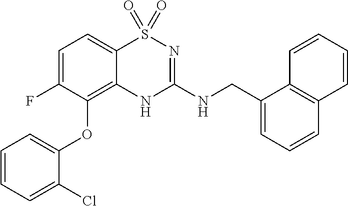 [SMILES: O=S1(=O)N=C(NCc2cccc3ccccc23)Nc2c1ccc(F)c2Oc1ccccc1Cl] | 0.566 | 481.1 | 482.1 | [M + H]⁺ | 1 |
| 2-7 | 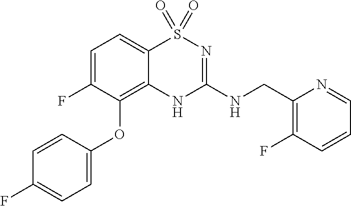 [SMILES: O=S1(=O)N=C(NCc2ncccc2F)Nc2c1ccc(F)c2Oc1ccc(F)cc1] | 0.433 | 434.1 | 435.1 | [M + H]⁺ | 1 |
| 2-8 | 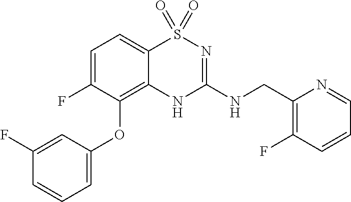 [SMILES: O=S1(=O)N=C(NCc2ncccc2F)Nc2c1ccc(F)c2Oc1cccc(F)c1] | 0.503 | 434.1 | 435.1 | [M + H]⁺ | 1 |
| 2-9 | 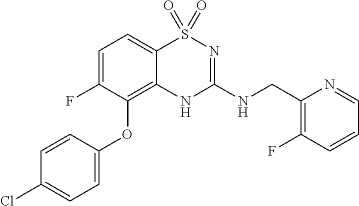 [SMILES: O=S1(=O)N=C(NCc2ncccc2F)Nc2c1ccc(F)c2Oc1ccc(Cl)cc1] | 0.529 | 450.0 | 451.1 | [M + H]⁺ | 1 |
| 2-10 | 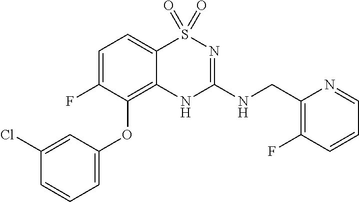 [SMILES: O=S1(=O)N=C(NCc2ncccc2F)Nc2c1ccc(F)c2Oc1cccc(Cl)c1] | 0.511 | 450.0 | 451.0 | [M + H]⁺ | 1 |
| 2-11 | 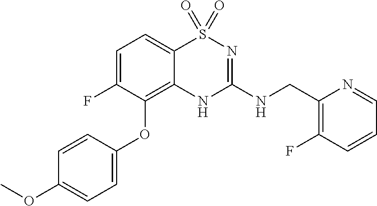 [SMILES: COc1ccc(Oc2c(F)ccc3c2NC(NCc2ncccc2F)=NS3(=O)=O)cc1] | 0.490 | 446.1 | 447.1 | [M + H]⁺ | 1 |
| 2-12 | 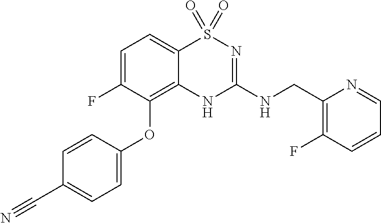 [SMILES: N#Cc1ccc(Oc2c(F)ccc3c2NC(NCc2ncccc2F)=NS3(=O)=O)cc1] | 0.513 | 441.1 | 442.2 | [M + H]⁺ | 2 |
| 2-13 | 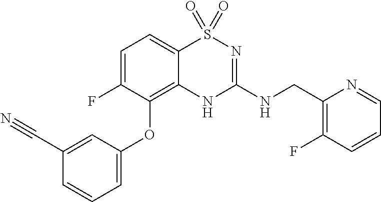 [SMILES: N#Cc1cccc(Oc2c(F)ccc3c2NC(NCc2ncccc2F)=NS3(=O)=O)c1] | 0.487 | 441.1 | 442.1 | [M + H]⁺ | 1 |
| 2-14 | 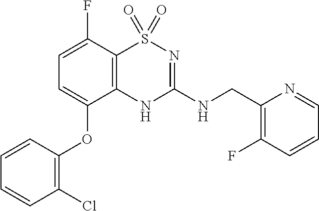 [SMILES: O=S1(=O)N=C(NCc2ncccc2F)Nc2c(Oc3ccccc3Cl)ccc(F)c21] | 0.512 | 450.1 | 451.1 | [M + H]⁺ | 1 |
| 2-15 | 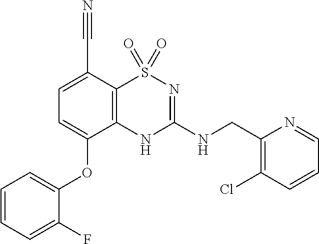 [SMILES: N#Cc1ccc(Oc2ccccc2F)c2c1S(=O)(=O)N=C(NCc1ncccc1Cl)N2] | 0.505 | 457.0 | 458.3 | [M + H]⁺ | 1 |

### Example 3

### Synthesis of Representative Compound 3-1

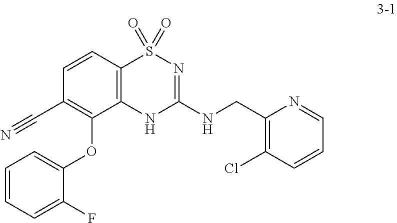 [SMILES: N#Cc1ccc2c(c1Oc1ccccc1F)NC(NCc1ncccc1Cl)=NS2(=O)=O]

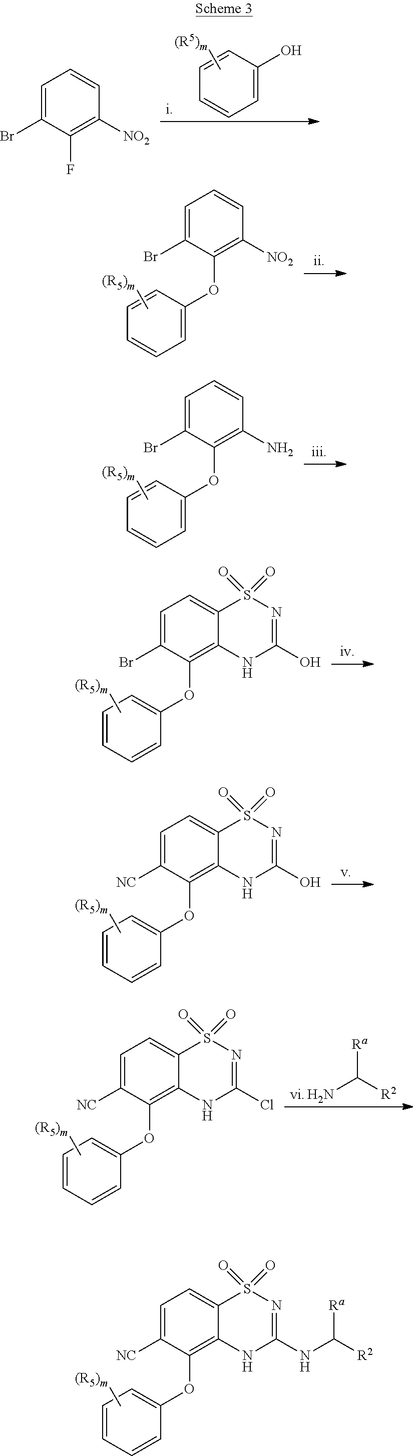

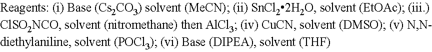

### Step 3-1. Synthesis of 1-bromo-2-(2-fluorophenoxy)-3-nitrobenzene (INT3A)

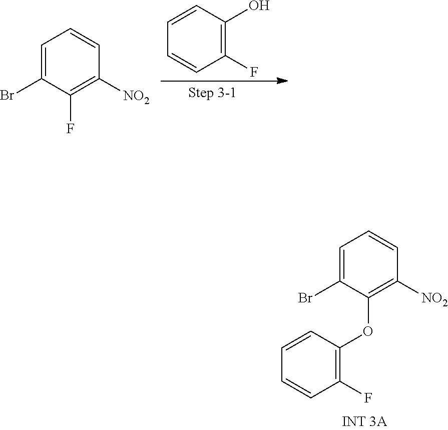 [SMILES: O=[N+]([O-])c1cccc(Br)c1F ; Oc1ccccc1F ; O=[N+]([O-])c1cccc(Br)c1Oc1ccccc1F]

To a solution of 1-bromo-2-fluoro-3-nitro-benzene (5.0 g, 23 mmol, 1 eq) and 2-fluorophenol (2.68 g, 23.9 mmol, 2.21 mL, 1.05 eq) in MeCN (50 mL), was added Cs₂CO₃ (16.3 g, 50.0 mmol, 2.2 eq). The reaction mixture was heated and stirred at 80° C. for 14 h. Upon cooling, the crude mixture was filtered through a pad of Celite and concentrated in vacuo. The crude residue was purified by silica gel chromatography (0á100% EtOAc/petroleum ether) to afford 1-bromo-2-(2-fluorophenoxy)-3-nitrobenzene (INT3A) (5.5 g, 15.0 mmol, 66% yield) that was used without further purification. ¹H NMR (400 MHz, DMSO-d6) δ=8.19 (d, J=8.1 Hz, 2H), 7.55 (t, J=8.2 Hz, 1H), 7.44-7.35 (m, 1H), 7.18-7.06 (m, 2H), 6.72 (dt, J=2.1, 8.2 Hz, 1H).

### Step 3-2. Synthesis of 3-bromo-2-(2-fluorophenoxy)aniline (INT3B)

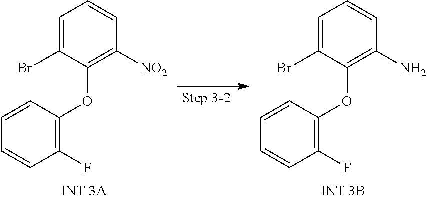 [SMILES: O=[N+]([O-])c1cccc(Br)c1Oc1ccccc1F ; Nc1cccc(Br)c1Oc1ccccc1F]

To a solution of 1-bromo-2-(2-fluorophenoxy)-3-nitro-benzene INT 3A (5.5 g, 17.6 mmol, 1 eq) in EtOAc (50 mL), was added SnCl₂·2H₂O (19.88 g, 88.12 mmol, 5 eq). The mixture was heated and stirred at 50° C. for 3 h. The reaction mixture was poured into an aq. sat. NaHCO₃ solution (300 mL) while stirring. The mixture was extracted with EtOAc (2×200 mL). The combined organic phase was washed with brine (3×80 mL), dried over Na₂SO₄, filtered, and concentrated in vacuo to give a residue. The crude residue was purified by silica gel chromatography (0 to 15% EtOAc/petroleum ether) to afford 3-bromo-2-(2-fluorophenoxy)aniline (4.5 g, 13.4 mmol, 76% yield) INT 3B that was used without further purification. ¹H NMR (400 MHz, DMSO-d6) δ=7.35-7.27 (m, 1H), 7.06-6.97 (m, 2H), 6.96-6.91 (m, 1H), 6.82 (d, J=8.1 Hz, 2H), 6.52-6.44 (m, 1H), 5.35 (s, 2H).

### Step 3-3. Synthesis of 6-bromo-5-(2-fluorophenoxy)-3-hydroxy-4H-benzo[e][1,2,4]thiadiazine 1,1-dioxide (INT 3C)

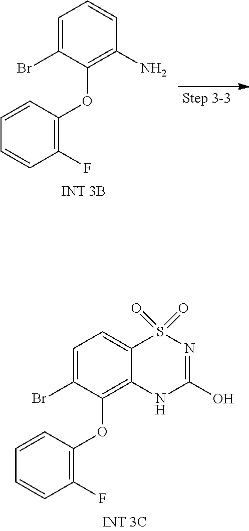 [SMILES: Nc1cccc(Br)c1Oc1ccccc1F ; O=S1(=O)N=C(O)Nc2c1ccc(Br)c2Oc1ccccc1F]

To a solution of N-(oxomethylene)sulfamoyl chloride (3.42 g, 24.2 mmol, 2.1 mL, 1.52 eq) in nitrobenzene (50 mL) at 0° C., was added 3-bromo-2-(2-fluorophenoxy)aniline INT 3B (4.5 g, 16 mmol, 1 eq). The mixture was stirred at 0° C. for 0.5 hr. Then, AlCl₃ (3.2 g, 24.0 mmol, 1.31 mL, 1.5 eq) was added. The mixture was heated and stirred at 120° C. for 1.5 h. The reaction solution was cooled to room temperature and then diluted with saturated NH₄Cl aqueous solution (500 mL). The aqueous layer was extracted with ethyl acetate (3×200 mL). The combined organic phase was washed with brine (200 mL), dried over anhydrous sodium sulfate, filtered and concentrated in vacuo to give a crude brown oil. The obtained brown oil was diluted with petroleum ether (300 mL) and then stirred for 30 min. The brown oil eventually formed a brown precipitate that was collected by vacuum filtration. The filter cake was washed with petroleum ether (200 mL) and then dried under vacuum to give a yellow solid. The yellow solid was triturated in EtOH (10 mL) and collected by filtration to afford 6-bromo-5-(2-fluorophenoxy)-1,1-dioxo-4H-1,2,4-benzothiadiazin-3-ol INT 3C (3.9 g, 9.0 mmol, 56% yield) that was used without further purification. ¹H NMR (400 MHz, DMSO-d6) δ=11.47 (s, 1H), 7.74-7.63 (m, 2H), 7.41-7.33 (m, 1H), 7.14-6.99 (m, 2H), 6.59 (t, J=8.0 Hz, 1H).

### Step 3-4. Synthesis of 5-(2-fluorophenoxy)-3-hydroxy-4H-benzo[e][1,2,4]thiadiazine-6-carbonitrile 1,1-dioxide (INT3D)

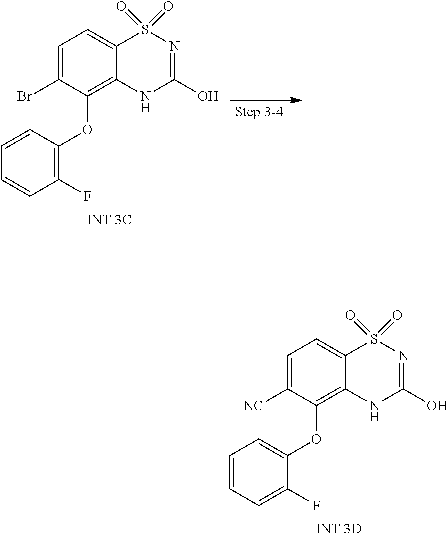 [SMILES: O=S1(=O)N=C(O)Nc2c1ccc(Br)c2Oc1ccccc1F ; N#Cc1ccc2c(c1Oc1ccccc1F)NC(O)=NS2(=O)=O]

To a solution of 6-bromo-5-(2-fluorophenoxy)-1,1-dioxo-4H-1,2,4-benzothiadiazin-3-ol INT 3C (1.58 g, 4.08 mmol, 1 eq) in DMSO (15 mL), was added CuCN (804 mg, 8.98 mmol, 1.96 mL, 2.2 eq). The mixture was stirred at 100° C. for 14 h. The mixture was then heated and stirred at 110° C. for 3 h. The reaction mixture was poured into ammonia water (1%, 40 mL) and a brown precipitate was formed. The mixture was filtered. The filtrate was extracted with EtOAc (2×30 mL). The aqueous phase was acidified with iN HCl to pH=5. The mixture was extracted with EtOAc (2×30 mL). The combined organic layer was washed with brine (3×30 mL), dried over Na₂SO₄, filtered, and concentrated under vacuum to give 5-(2-fluorophenoxy)-3-hydroxy-1,1-dioxo-4H-1,2,4-benzothiadiazine-6-carbonitrile INT 3D (500 mg, 945 mol, 23% yield) that was used without further purification.

### Step 3-5. Synthesis of 3-chloro-5-(2-fluorophenoxy)-4H-benzo[e][1,2,4]thiadiazine-6-carbonitrile 1,1-dioxide (INT 3E)

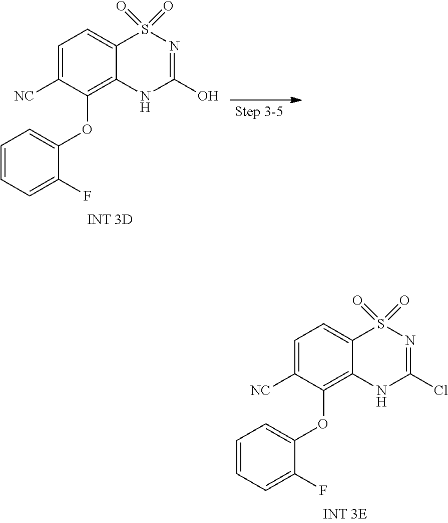 [SMILES: N#Cc1ccc2c(c1Oc1ccccc1F)NC(O)=NS2(=O)=O ; N#Cc1ccc2c(c1Oc1ccccc1F)NC(Cl)=NS2(=O)=O]

To a solution of 5-(2-fluorophenoxy)-3-hydroxy-1,1-dioxo-4H-1,2,4-benzothiadiazine-6-carbonitrile INT 3D (500 mg, 1.50 mmol, 1 eq) in POCl₃ (5 mL), was added N,N-diethylaniline (373 mg, 2.50 mmol, 0.4 mL, 1.67 eq). The reaction mixture was heated and stirred at 120° C. for 2 h. The reaction solution was poured into water (50 mL) portionwise while the temperature was kept below 35° C. The obtained mixture was stirred for 1 h. The mixture was extracted with EtOAc (3×50 mL). The combined organic layer was washed with brine (2×60 mL), dried over Na₂SO₄, filtered, and concentrated in vacuo to give crude material. The crude material was purified by prep-TLC silica gel (3:1 EtOAc/petroleum ether) to afford 3-chloro-5-(2-fluorophenoxy)-1,1-dioxo-4H-1,2,4-benzothiadiazine-6-carbonitrile INT 3E (150 mg, 332 mol, 22% yield). The aqueous mixture was adjusted to pH=7 by KOH and then discarded.

### Step 3-6. Synthesis of 3-(((3-chloropyridin-2-yl)methyl)amino)-5-(2-fluorophenoxy)-4H-benzo[e][1,2,4]thiadiazine-6-carbonitrile 1,1-dioxide (Compound 3-1)

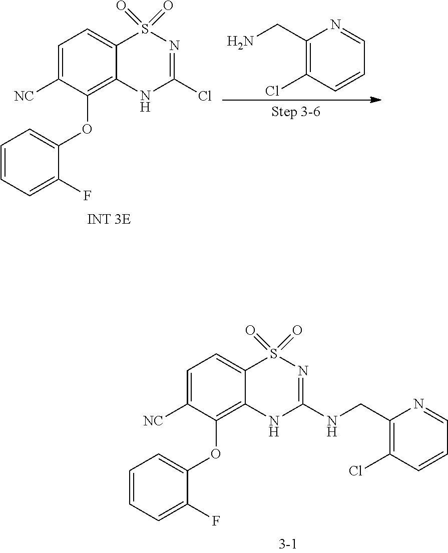 [SMILES: N#Cc1ccc2c(c1Oc1ccccc1F)NC(Cl)=NS2(=O)=O ; NCc1ncccc1Cl ; N#Cc1ccc2c(c1Oc1ccccc1F)NC(NCc1ncccc1Cl)=NS2(=O)=O]

To a solution of (3-chloro-2-pyridyl)methanamine INT 3E (182 mg, 1.28 mmol, 3 eq) and 3-chloro-5-(2-fluorophenoxy)-1,1-dioxo-4H-1,2,4-benzothiadiazine-6-carbonitrile (150 mg, 426 mol, 1 eq) in THE (5 mL) was added DIPEA (275.58 mg, 2.13 mmol, 371.40 μL, 5 eq). The mixture was stirred at 25° C. for 16 h. The reaction mixture was poured into water (30 mL). The product was extracted with EtOAc (2×30 mL). The combined organic layer was washed with brine (3×30 mL), dried over Na₂SO₄, filtered, and concentrated in vacuo to give a residue. The residue was purified by preparative HPLC (column: Waters Xbridge 150×25 mm×5 um; mobile phase: [A-B: water(NH₄HCO₃)-ACN]; gradient: 28%-58% B over 9 min) and lyophilized to afford 3-(((3-chloropyridin-2-yl)methyl)amino)-5-(2-fluorophenoxy)-4H-benzo[e][1,2,4]thiadiazine-6-carbonitrile 1,1-dioxide (3-1) (33.3 mg, 72.7 μmol, 17% yield). LCMS (m/z) calculated for C₂₀H₁₃ClF₂N₅O₃S: 457.0; found 458.2 [M+H]+, tR=0.516 min (Purity Method 1). ¹H NMR (400 MHz, DMSO-d6) δ=11.13 (s, 2H), 8.54 (br d, J=4.4 Hz, 1H), 8.23 (br t, J=4.1 Hz, 1H), 8.00 (d, J=8.1 Hz, 1H), 7.81-7.69 (m, 2H), 7.48-7.41 (m, 2H), 7.26 (br d, J=1.0 Hz, 1H), 7.27-7.20 (m, 1H), 7.17 (t, J=7.8 Hz, 1H), 7.05-6.96 (m, 1H), 4.70 (d, J=4.4 Hz, 2H).

¹H NMR (400 MHz, METHANOL-d4) δ=8.53-8.42 (m, 1H), 7.87 (br d, J=7.9 Hz, 1H), 7.79 (br d, J=8.3 Hz, 1H), 7.68-7.53 (m, 1H), 7.38-7.26 (m, 2H), 7.25-7.06 (m, 2H), 6.99-6.88 (m, 1H), 4.77 (br s, 2H).

### Synthesis of Scheme 3 Compounds

Alternatively for step 3-5, the following compounds were made using 1.3 Eq of BOP reagent with 2 Eq of DBU as base and DMF as solvent at 80° C. for 15 h: 3-2.

The compounds listed in Table 4 were made using the procedures of Scheme 3.

TABLE 4

| | | Purity | Exact | | | |
|---|---|---|---|---|---|---|
| Cpd | | RT | mass | observed | | Purity |
| No. | Structure | (min) | (g/mol) | m/z | Ion | Method |
| 3-1 | 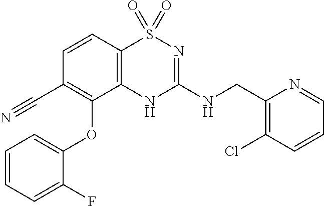 [SMILES: N#Cc1ccc2c(c1Oc1ccccc1F)NC(NCc1ncccc1Cl)=NS2(=O)=O] | 0.512 | 457.0 | 458.2 | [M + H]⁺ | 1 |
| 3-2 | 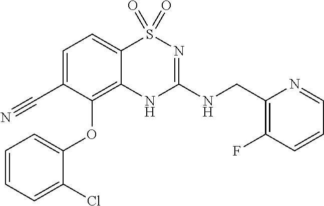 [SMILES: N#Cc1ccc2c(c1Oc1ccccc1Cl)NC(NCc1ncccc1F)=NS2(=O)=O] | 0.505 | 457.0 | 458.1 | [M + H]⁺ | 1 |

### Example 4

### Synthesis of Representative Compound 4-1

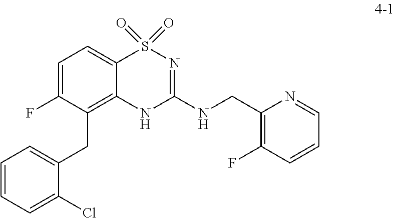 [SMILES: O=S1(=O)N=C(NCc2ncccc2F)Nc2c1ccc(F)c2Cc1ccccc1Cl]

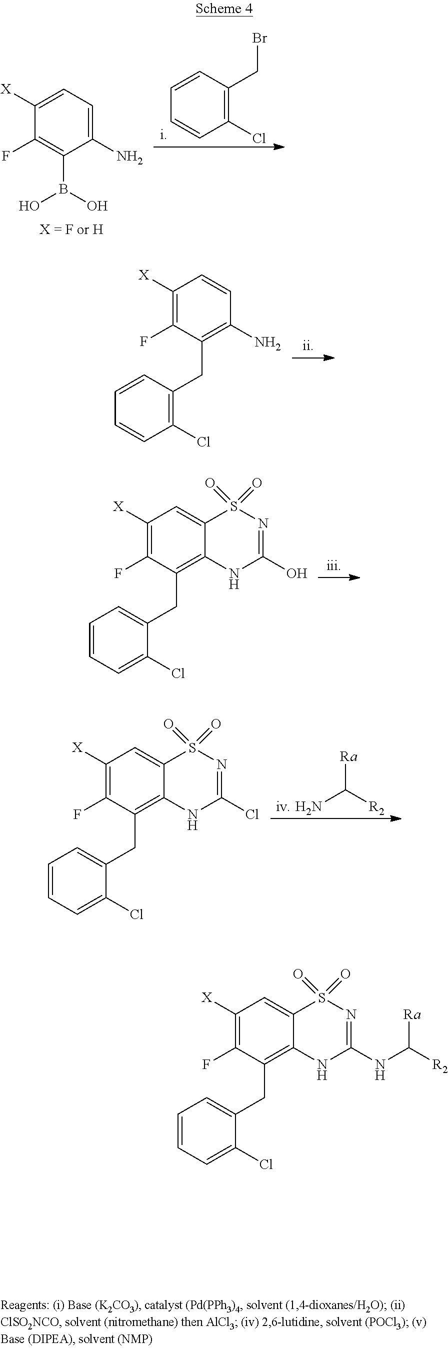

### Step 4-1. Synthesis of 2-(2-chlorobenzyl)-3-fluoroaniline (INT 4A)

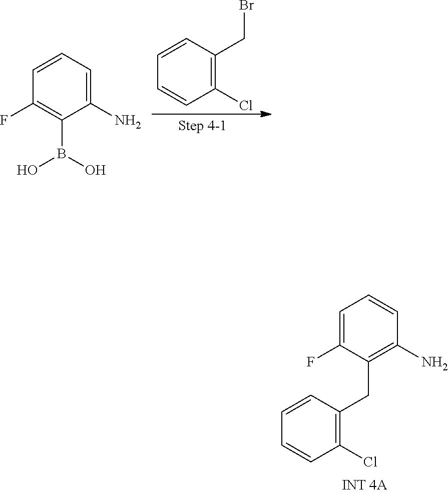 [SMILES: Nc1cccc(F)c1B(O)O ; Clc1ccccc1CBr ; Nc1cccc(F)c1Cc1ccccc1Cl]

To a solution of (2-amino-6-fluorophenyl)boronic acid (980 mg, 1.3 Eq, 6.32 mmol) and 1-(bromomethyl)-2-chlorobenzene (1000 mg, 1 Eq, 4.9 mmol) in 1,4-dioxane (6 mL) and water (4 mL), potassium carbonate (2.02 g, 3 Eq, 14.6 mmol) and tetrakis(triphenylphosphine)palladium(0) (56 mg, 0.01 Eq, 49 mol) were added. The reaction mixture was degassed and then heated at 100° C. while stirring for 3 h. The reaction mixture was passed through a syringe filter, concentrated in vacuo and purified by silica gel chromatography (0á100% 10% MeOH in EtOAc/hexanes) to afford 2-(2-chlorobenzyl)-3-fluoroaniline INT 4A (789 mg, 3.35 mmol, 69% yield). LCMS (m/z) calculated for C₁₃H₁₁ClFN: 235.0; found 236.1 [M+H]+, tR=5.67 min (Purity method 4). ¹H NMR (400 MHz, DMSO) δ 7.56-7.33 (m, 1H), 7.21 (tt, J=7.4, 5.5 Hz, 2H), 7.01 (td, J=8.1, 6.7 Hz, 1H), 6.80 (dd, J=7.0, 2.3 Hz, 1H), 6.54 (d, J=8.1 Hz, 1H), 6.36 (ddd, J=9.5, 8.1, 1.1 Hz, 1H), 5.26 (s, 2H), 3.92 (d, J=1.9 Hz, 2H).

### Step 4-2. Synthesis of 5-(2-chlorobenzyl)-6-fluoro-3-hydroxy-4H-benzo[e][1,2,4]thiadiazine 1,1-dioxide (INT 4B)

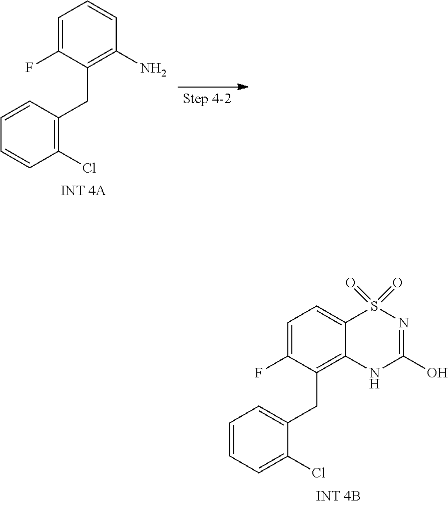 [SMILES: Nc1cccc(F)c1Cc1ccccc1Cl ; O=S1(=O)N=C(O)Nc2c1ccc(F)c2Cc1ccccc1Cl]

To sulfurisocyanatidic chloride (540 mg, 1.2 Eq, 3.82 mmol) was added nitromethane (5 mL). The mixture was cooled to 0° C. A solution of INT 4A (750 mg, 1 Eq, 3.18 mmol) in nitromethane (5 mL) was added dropwise. The reaction mixture was stirred at 0° C. for 15 minutes and then aluminum trichloride (1.70 g, 4 Eq, 12.7 mmol) was added. The reaction mixture was heated to 115° C. for 3 hours. The reaction mixture was cooled to room temperature and poured into ice cold water (20 mL). The resultant precipitate was filtered, washed with water (3×5 mL) then hexane (3×5 mL). The crude material was purified by silica gel chromatography (0á100% 10% MeOH in EtOAc/hexanes) to afford obtained 5-(2-chlorobenzyl)-6-fluoro-3-hydroxy-4H-benzo[e][1,2,4]thiadiazine 1,1-dioxide INT 4B (451 mg, 1.32 mmol, 42% yield).

### Step 4-3. Synthesis of 3-chloro-5-(2-chlorobenzyl)-6-fluoro-4H-benzo[e][1,2,4]thiadiazine 1,1-dioxide (INT 4C)

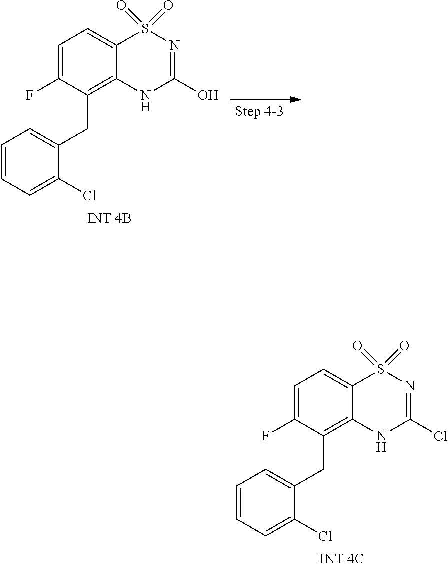 [SMILES: O=S1(=O)N=C(O)Nc2c1ccc(F)c2Cc1ccccc1Cl ; O=S1(=O)N=C(Cl)Nc2c1ccc(F)c2Cc1ccccc1Cl]

To INT 4B (550 mg, 1 Eq, 1.61 mmol) in a sealed tube was added POCl₃ (2.47 g, 1.50 mL, 10 Eq, 16.1 mmol) and 2,6-dimethylpyridine (173 mg, 1 Eq, 1.61 mmol). The tube was sealed and heated at 105° C. while stirring for 18 h. The reaction mixture was concentrated in vacuo to afford 3-chloro-5-(2-chlorobenzyl)-6-fluoro-4H-benzo[e][1,2,4]thiadiazine 1,1-dioxide INT 4C that was used without further purification. LCMS (m/z) calculated for C₁₄H₉Cl₂FN₂O₂S: 358.0; found 358.2 [M]⁺, t_{R}=5.04 min (Purity method 3).

### Step 4-4. Synthesis of 6-fluoro-5-(2-fluorobenzyl)-3-(((3-fluoropyridin-2-yl)methyl)amino)-4H-benzo[e][1,2,4]thiadiazine 1,1-dioxide (Compound 4-1)

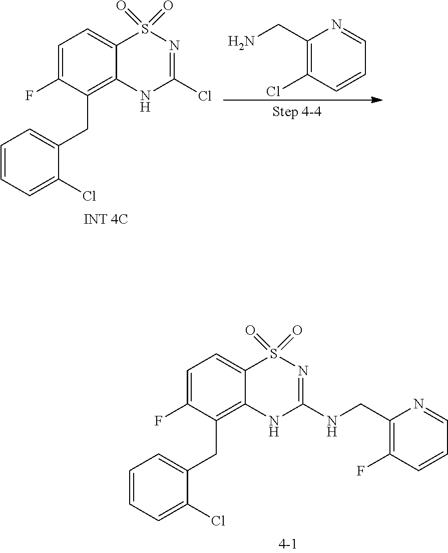 [SMILES: O=S1(=O)N=C(Cl)Nc2c1ccc(F)c2Cc1ccccc1Cl ; NCc1ncccc1Cl ; O=S1(=O)N=C(NCc2ncccc2F)Nc2c1ccc(F)c2Cc1ccccc1Cl]

To a solution of 3-chloro-5-(2-chlorobenzyl)-6-fluoro-4H-benzo[e][1,2,4]thiadiazine 1,1-dioxide INT 4C (20 mg, 1 Eq, 56 mol) in NMP (2 mL) in a sealed tube was added (3-fluoropyridin-2-yl)methanamine (8.4 mg, 1.2 Eq, 67 mol) and N-ethyl-N-isopropylpropan-2-amine (22 mg, 3 Eq, 0.17 mmol). The tube was sealed and heated at 120° C. while stirring for 3 h. The reaction mixture was cooled to room temperature and purified by reversed phase HPLC (A-B gradient: 35-55%, A: 0.1% formic acid in MeCN and B: 0.1% formic acid in H₂O) to afford 5-(2-chlorobenzyl)-6-fluoro-3-(((3-fluoropyridin-2-yl)methyl)amino)-4H-benzo[e][1,2,4]thiadiazine 1,1-dioxide 4-1 (9.3 mg, 21 μmol, 37% yield). LCMS (m/z) calculated for C₂₀H₁₅CF₂N₄O₂S: 448.1, found 449.0 [M+H]⁺, t_{R}=8.537 μm (Purity method 3). ¹H NMR (400 MHz, DMSO) δ 10.30 (s, 1H), 8.42 (s, 1H), 7.96 (s, 1H), 7.79 (ddd, J=10.9, 7.7, 5.3 Hz, 2H), 7.55 (dd, J=7.6, 1.6 Hz, 1H), 7.48 (dt, J=8.7, 4.5 Hz, 1H), 7.38-7.10 (m, 3H), 6.83-6.60 (m, 1H), 4.67 (d, J=5.2 Hz, 2H), 4.19 (s, 2H).

### Synthesis of Scheme 4 Compounds

The compounds listed in Table 5 were made using the procedures of Scheme 4.

TABLE 5

| | | Purity | Exact | | | |
|---|---|---|---|---|---|---|
| Cpd | | RT | mass | observed | | Purity |
| No. | Structure | (min) | (g/mol) | m/z | Ion | Method |
| 4-1 | 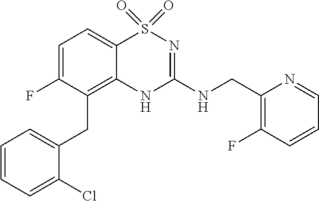 [SMILES: O=S1(=O)N=C(NCc2ncccc2F)Nc2c1ccc(F)c2Cc1ccccc1Cl] | 8.582 | 448.1 | 449 | [M + H]⁺ | 3 |
| 4-2 | 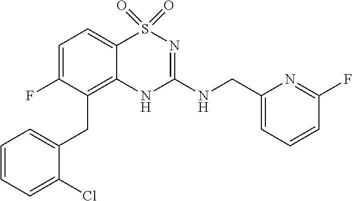 [SMILES: O=S1(=O)N=C(NCc2cccc(F)n2)Nc2c1ccc(F)c2Cc1ccccc1Cl] | 8.701 | 448.1 | 449 | [M + H]⁺ | 3 |
| 4-3 | 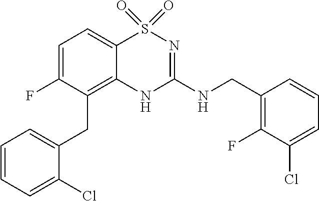 [SMILES: O=S1(=O)N=C(NCc2cccc(Cl)c2F)Nc2c1ccc(F)c2Cc1ccccc1Cl] | 9.797 | 481.0 | 482 | [M + H]⁺ | 3 |
| 4-4 | 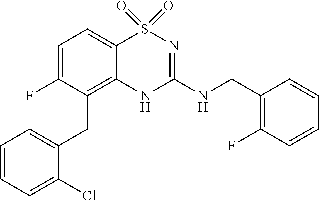 [SMILES: O=S1(=O)N=C(NCc2ccccc2F)Nc2c1ccc(F)c2Cc1ccccc1Cl] | 9.308 | 447.1 | 448 | [M + H]⁺ | 3 |
| 4-5 | 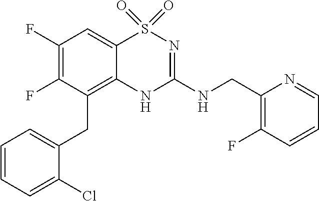 [SMILES: O=S1(=O)N=C(NCc2ncccc2F)Nc2c1cc(F)c(F)c2Cc1ccccc1Cl] | 9.068 | 466.0 | 467 | [M + H]⁺ | 3 |
| 4-6 | 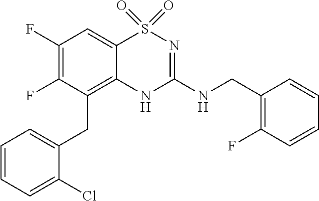 [SMILES: O=S1(=O)N=C(NCc2ccccc2F)Nc2c1cc(F)c(F)c2Cc1ccccc1Cl] | 9.739 | 465.1 | 466 | [M + H]⁺ | 3 |
| 4-7 | 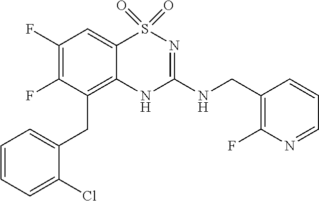 [SMILES: O=S1(=O)N=C(NCc2cccnc2F)Nc2c1cc(F)c(F)c2Cc1ccccc1Cl] | 8.716 | 466.0 | 467 | [M + H]⁺ | 3 |
| 4-8 | 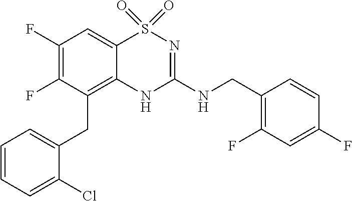 [SMILES: O=S1(=O)N=C(NCc2ccc(F)cc2F)Nc2c1cc(F)c(F)c2Cc1ccccc1Cl] | 9.886 | 483.0 | 484 | [M + H]⁺ | 3 |
| 4-9 | 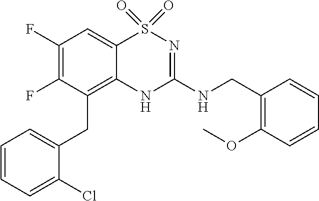 [SMILES: COc1ccccc1CNC1=NS(=O)(=O)c2cc(F)c(F)c(Cc3ccccc3Cl)c2N1] | 8.742 | 477.1 | 479 | [M + H]⁺ | 3 |
| 4-10 | 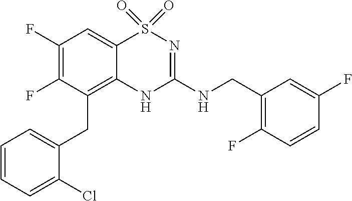 [SMILES: O=S1(=O)N=C(NCc2cc(F)ccc2F)Nc2c1cc(F)c(F)c2Cc1ccccc1Cl] | 9.769 | 483.0 | 484 | [M + H]⁺ | 3 |
| 4-11 | 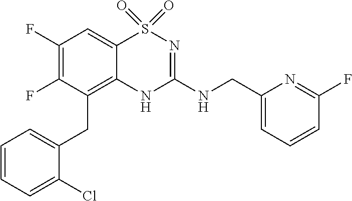 [SMILES: O=S1(=O)N=C(NCc2cccc(F)n2)Nc2c1cc(F)c(F)c2Cc1ccccc1Cl] | 8.207 | 466.0 | 467 | [M + H]⁺ | 3 |
| 4-12 | 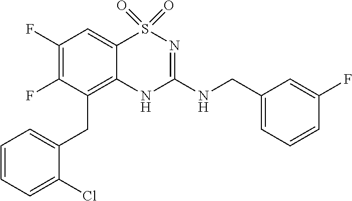 [SMILES: O=S1(=O)N=C(NCc2cccc(F)c2)Nc2c1cc(F)c(F)c2Cc1ccccc1Cl] | 9.776 | 465.1 | 466 | [M + H]⁺ | 3 |
| 4-13 | 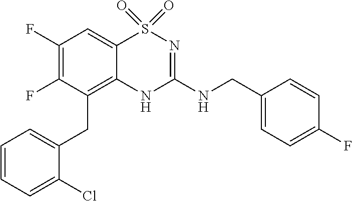 [SMILES: O=S1(=O)N=C(NCc2ccc(F)cc2)Nc2c1cc(F)c(F)c2Cc1ccccc1Cl] | 9.72 | 465.1 | 466 | [M + H]⁺ | 3 |
| 4-14 | 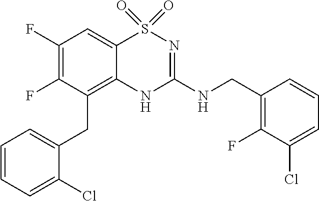 [SMILES: O=S1(=O)N=C(NCc2cccc(Cl)c2F)Nc2c1cc(F)c(F)c2Cc1ccccc1Cl] | 10.129 | 499.0 | 500 | [M + H]⁺ | 3 |
| 4-15 | 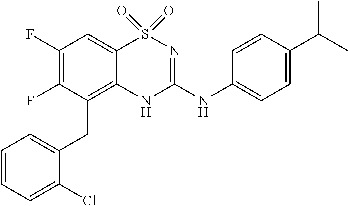 [SMILES: CC(C)c1ccc(NC2=NS(=O)(=O)c3cc(F)c(F)c(Cc4ccccc4Cl)c3N2)cc1] | 10.842 | 475.1 | 476 | [M + H]⁺ | 3 |
| 4-16 | 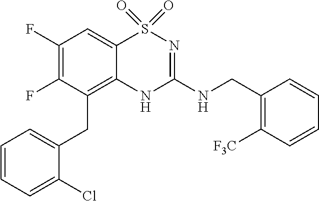 [SMILES: O=S1(=O)N=C(NCc2ccccc2C(F)(F)F)Nc2c1cc(F)c(F)c2Cc1ccccc1Cl] | 10.282 | 515.0 | 516 | [M + H]⁺ | 3 |
| 4-17 | 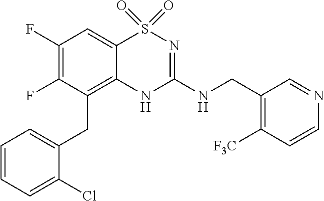 [SMILES: O=S1(=O)N=C(NCc2cnccc2C(F)(F)F)Nc2c1cc(F)c(F)c2Cc1ccccc1Cl] | 9.049 | 516.0 | 517 | [M + H]⁺ | 3 |
| 4-18 | 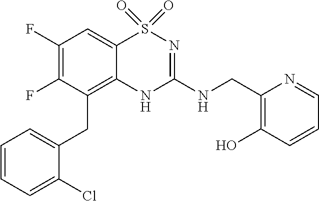 [SMILES: O=S1(=O)N=C(NCc2ncccc2O)Nc2c1cc(F)c(F)c2Cc1ccccc1Cl] | 7.143 | 464.1 | 465 | [M + H]⁺ | 3 |
| 4-19 | 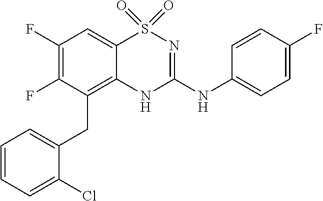 [SMILES: O=S1(=O)N=C(Nc2ccc(F)cc2)Nc2c1cc(F)c(F)c2Cc1ccccc1Cl] | 9.639 | 451.0 | 452 | [M + H]⁺ | 3 |
| 4-20 | 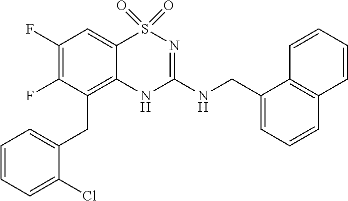 [SMILES: O=S1(=O)N=C(NCc2cccc3ccccc23)Nc2c1cc(F)c(F)c2Cc1ccccc1Cl] | 10.344 | 497.1 | 498.2 | [M + H]⁺ | 3 |

### Example 5

### Synthesis of Representative Compound 5-1

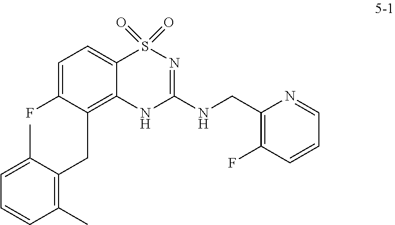 [SMILES: Cc1cccc(C)c1Cc1c(F)ccc2c1NC(NCc1ncccc1F)=NS2(=O)=O]

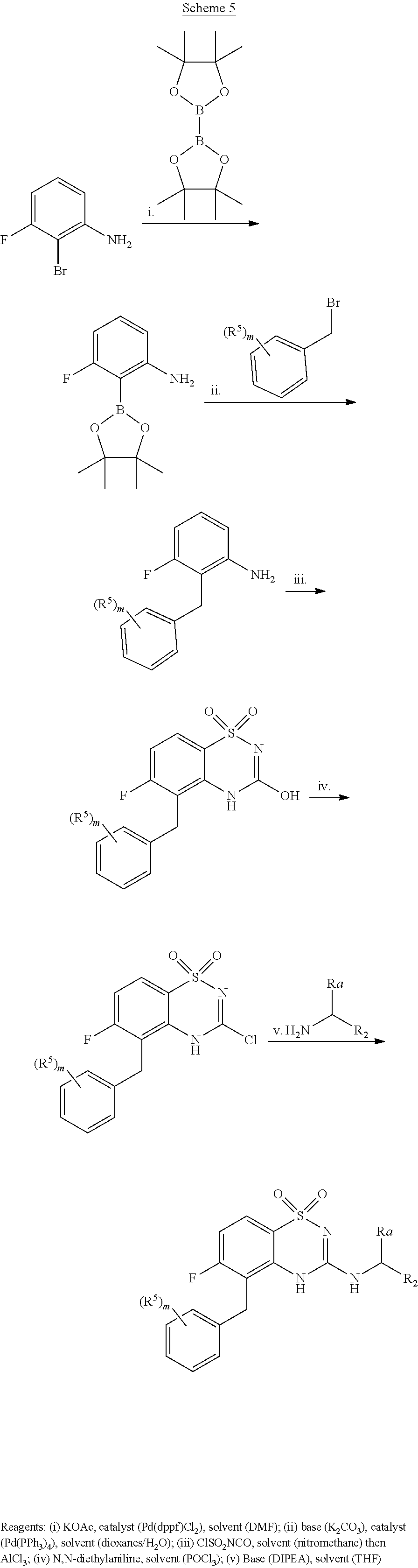

### Step 5-1. Synthesis of 3-fluoro-2-(4,4,5,5-tetramethyl-1,3,2-dioxaborolan-2-yl)aniline (INT 5A)

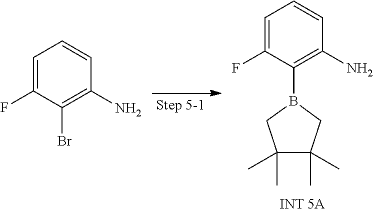 [SMILES: Nc1cccc(F)c1Br ; CC1(C)CB(c2c(N)cccc2F)CC1(C)C]

To a solution of 2-bromo-3-fluoro-aniline (10 g, 52.6 mmol, 1 eq) and 4,4,5,5-tetramethyl-2-(4,4,5,5-tetramethyl1,3,2-dioxaborolan-2-yl)-1,3,2-dioxaborolane (26.7 g, 105 mmol, 2 eq) in DMF (200 mL) were added KOAc (25.8 g, 263 mmol, 5 eq) and Pd(dppf)Cl₂ (3.85 g, 5.26 mmol, 0.1 eq). The reaction mixture was heated and stirred at 80° C. for 15 h. The reaction mixture was filtered and concentrated in vacuo to give a residue. The residue was purified by silica gel chromatography (0á50% EtOAc and petroleum ether) to afford 3-fluoro-2-(4,4,5,5-tetramethyl-1,3,2-dioxaborolan-2-yl)aniline INT 5A (12 g, 45.6 mmol, 87% yield) that was used without further purification. ¹H NMR (400 MHz, DMSO-d6) δ=7.14-7.06 (m, 1H), 6.39 (d, J=8.3 Hz, 1H), 6.15 (dd, J=8.3, 9.6 Hz, 1H), 5.83 (s, 2H), 1.28 (s, 12H).

### Step 5-2. Synthesis of 2-(2,6-dimethylbenzyl)-3-fluoroaniline (INT 5B)

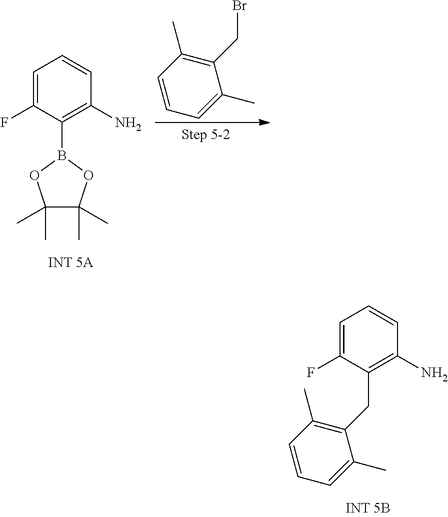

To a solution of 2-(bromomethyl)-1,3-dimethyl-benzene (5.04 g, 25.3 mmol, 1.2 eq) and 3-fluoro-2-(4,4,5,5-tetramethyl-1,3,2-dioxaborolan-2-yl)aniline INT 5A (5.0 g, 21.1 mmol, 1 eq) in dioxane (40 mL) and H₂O (10 mL) were added K₂CO₃ (5.83 g, 42.2 mmol, 2 eq) and Pd(PPh₃)₄ (2.44 g, 2.11 mmol, 0.1 eq). The mixture was heated and stirred at 80° C. for 15 h. The reaction mixture was cooled, filtered and concentrated in vacuo to give a residue. The residue was purified by silica gel chromatography (0á100% EtOAc/petroleum ether) to afford 2-(2,6-dimethylbenzyl)-3-fluoroaniline INT 5B (800 mg, 3.14 mmol, 15% yield) that was used without further purification.

### Step 5-3. Synthesis of 5-(2,6-dimethylbenzyl)-6-fluoro-3-hydroxy-4H-benzo[e][1,2,4]thiadiazine 1,1-dioxide (INT 5C)

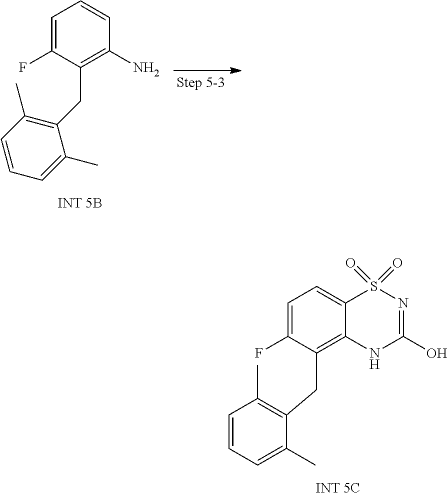 [SMILES: Cc1cccc(C)c1Cc1c(N)cccc1F ; Cc1cccc(C)c1Cc1c(F)ccc2c1NC(O)=NS2(=O)=O]

To a solution of N-(oxomethylene)sulfamoyl chloride (648.13 mg, 4.58 mmol, 398.60 μL, 1.4 eq) in nitrobenzene (5 mL) was added 2-[(2,6-dimethylphenyl)methyl]-3-fluoro-aniline (750 mg, 3.27 mmol, 1 eq) at 0° C. for 0.5 h. Then AlCl₃ (654.23 mg, 4.91 mmol, 268.13 L, 1.5 eq) was added. The mixture was stirred at 60° C. for 1.5 h. The reaction mixture was quenched by the addition of water 50 mL, and then adjusted to pH>7 with sodium hydroxide. A brown precipitate was formed. It was collected by filtration and dried under high vacuum to afford 5-(2,6-dimethylbenzyl)-6-fluoro-3-hydroxy-4H-benzo[e][1,2,4]thiadiazine 1,1-dioxide INT 5C (400 mg, crude) that was used without further purification.

### Step 5-4. Synthesis of 3-chloro-5-(2,6-dimethylbenzyl)-6-fluoro-4H-benzo[e][1,2,4]thiadiazine 1,1-dioxide (INT 5D)

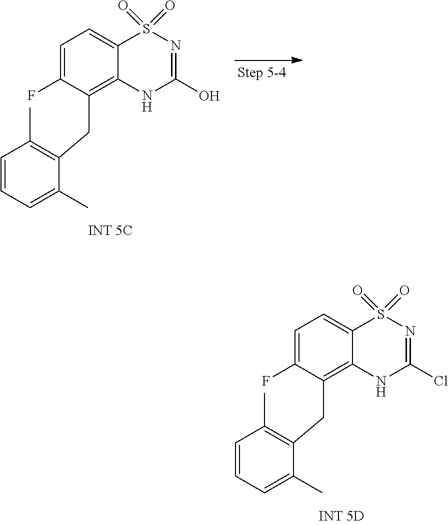 [SMILES: Cc1cccc(C)c1Cc1c(F)ccc2c1NC(O)=NS2(=O)=O ; Cc1cccc(C)c1Cc1c(F)ccc2c1NC(Cl)=NS2(=O)=O]

A mixture of 5-[(2,6-dimethylphenyl)methyl]-6-fluoro-1,1-dioxo-4H-1,2,4-benzothiadiazin-3-ol INT 5C (330 mg, 986.95 mol, 1 eq) and N,N-diethylaniline (307.88 mg, 2.06 mmol, 329.99 μL, 2.09 eq) in POCl₃ (5 mL) was heated and stirred at 120° C. for 4 h. The mixture was concentrated in vacuo, the residue was slowly quenched by saturated NaHCO₃ aqueous solution to pH ˜8, then extracted EtOAc (3×30 mL). The combined organic layer was dried over anhydrous sodium sulfate, filtered, and concentrated in vacuo. The crude was purified by prep-TLC (2/1 EtOAc/petroleum ether) to afford 3-chloro-5-(2,6-dimethylbenzyl)-6-fluoro-4H-benzo[e][1,2,4]thiadiazine 1,1-dioxide INT 5D (150 mg, 140 μmol, 14% yield) that was used without further purification.

### Step 5-5. Synthesis of 5-(2,6-dimethylbenzyl)-6-fluoro-3-(((3-fluoropyridin-2-yl)methyl)amino)-4H-benzo[e][1,2,4]thiadiazine 1,1-dioxide (Compound 5-1)

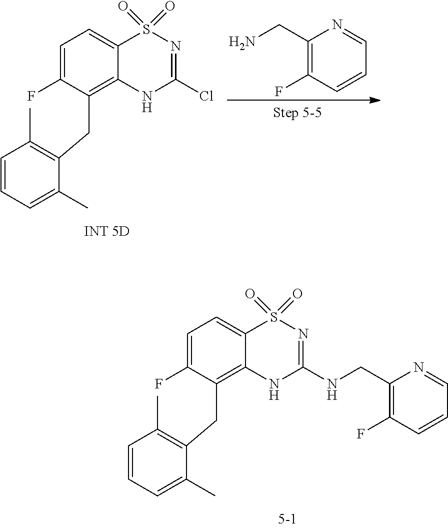 [SMILES: Cc1cccc(C)c1Cc1c(F)ccc2c1NC(Cl)=NS2(=O)=O ; NCc1ncccc1F ; Cc1cccc(C)c1Cc1c(F)ccc2c1NC(NCc1ncccc1F)=NS2(=O)=O]

To a solution of 3-chloro-5-[(2,6-dimethylphenyl)methyl]-6-fluoro-4H-1,2,4-benzothiadiazine 1,1-dioxide INT 5D (150.0 mg, 140.30 mol, 1 eq) in THE (5 mL) and DIPEA (35 mg, 271 mol, 47.2 μL, 1.93 eq) was added (3-fluoro-2-pyridyl)methanamine (25 mg, 198 mol, 1.41 eq). The reaction mixture was heated and stirred at 80° C. for 15 h. The crude was purified by prep-HPLC (column: Waters Xbridge 150×25 mm×5 um; A-B mobile phase: [water (NH₄HCO₃)-ACN]; gradient: 35%-65% B over 9 min) to afford 5-(2,6-dimethylbenzyl)-6-fluoro-3-(((3-fluoropyridin-2-yl)methyl)amino)-4H-benzo[e][1,2,4]thiadiazine 1,1-dioxide 5-1 (15 mg, 33 mol, 24% yield). LCMS (m/z) calculated for C₂₂H₂₀F₂N₄O₂S: 442.1; found 443.2 [M+H]⁺, t_{R}=0.580 min (Purity Method 2). ¹H NMR (400 MHz, DMSO-d6) δ=10.53 (br s, 1H), 8.47 (d, J=4.6 Hz, 1H), 8.17 (br s, 1H), 7.80 (t, J=9.3 Hz, 1H), 7.64 (br dd, J=5.6, 8.3 Hz, 1H), 7.49 (td, J=4.4, 8.4 Hz, 1H), 7.07-6.90 (m, 4H), 4.72 (br d, J=4.0 Hz, 2H), 4.08 (s, 2H), 2.18 (s, 6H).

¹⁹F NMR (376 MHz, DMSO-d6) δ=−106.26 (s, 1F), −126.41 (s, 1F).

### Synthesis of Scheme 5 Compounds

The compounds listed in Table 6 were made using the procedures of Scheme 5.

TABLE 6

| | | Purity | Exact | | | |
|---|---|---|---|---|---|---|
| Cpd | | RT | mass | observed | | Purity |
| No. | Structure | (min) | (g/mol) | m/z | Ion | Method |
| 5-1 | 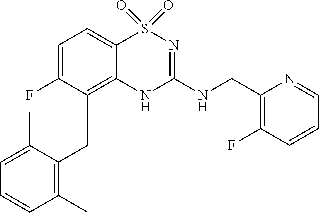 [SMILES: Cc1cccc(C)c1Cc1c(F)ccc2c1NC(NCc1ncccc1F)=NS2(=O)=O] | 0.580 | 442.1 | 443.2 | [M + H]⁺ | 2 |

### Example 6

### Synthesis of Representative Compound 6-1

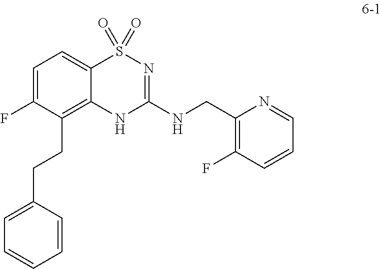 [SMILES: O=S1(=O)N=C(NCc2ncccc2F)Nc2c1ccc(F)c2CCc1ccccc1]

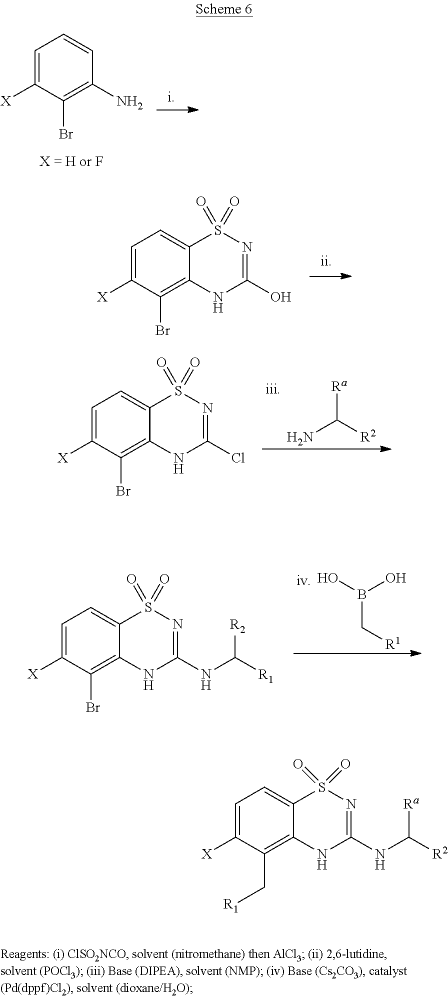

### Step 6-1. Synthesis of 5-bromo-6-fluoro-3-hydroxy-4H-benzo[e][1,2,4]thiadiazine 1,1-dioxide (INT 6A)

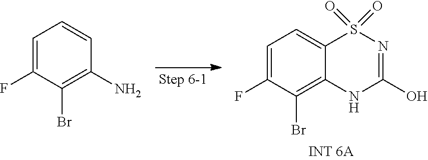 [SMILES: Nc1cccc(F)c1Br ; O=S1(=O)N=C(O)Nc2c1ccc(F)c2Br]

To a solution of sulfurisocyanatidic chloride (0.89 g, 1.2 Eq, 6.3 mmol) in nitromethane (15 mL) at 0° C. was added a solution of 2-bromo-3-fluoroaniline (1.0 g, 1 Eq, 5.3 mmol) in nitromethane (15 mL) dropwise. The reaction mixture was stirred at 0° C. for 15 minutes and then aluminum trichloride (2.1 g, 3 Eq, 16 mmol) was added. The reaction mixture was heated at 105° C. for 3 h. The reaction mixture was cooled to room temperature and added to ice cold water (50 mL). The resultant precipitate was filtered, washed with water (3×15 mL) and hexane (3×15 mL) to afford 5-bromo-6-fluoro-3-hydroxy-4H-benzo[e][1,2,4]thiadiazine 1,1-dioxide (INT 6A) that was used without further purification.

### Step 6-2. Synthesis of 5-bromo-3-chloro-6-fluoro-4H-benzo[e][1,2,4]thiadiazine 1,1-dioxide (INT 6B)

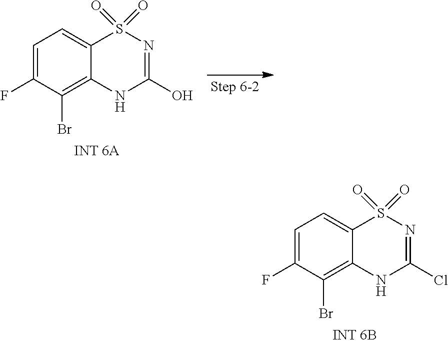 [SMILES: O=S1(=O)N=C(O)Nc2c1ccc(F)c2Br ; O=S1(=O)N=C(Cl)Nc2c1ccc(F)c2Br]

To crude 5-bromo-3-chloro-6-fluoro-4H-benzo[e][1,2,4]thiadiazine 1,1-dioxide INT 6A was added POCl₃ (7.4 mL, 15 Eq, 79 mmol) and 2,6-lutidine (0.73 mL, 1.2 equivalents, 6.3 mmol). The reaction vessel was sealed and heated at 130° C. with stirring overnight. The reaction mixture was cooled to room temperature and poured into ice cold water (30 mL). The resultant precipitate was filtered, washed with water (3×10 mL) and hexane (3×10 mL) to afford 5-bromo-3-chloro-6-fluoro-4H-benzo[e][1,2,4]thiadiazine 1,1-dioxide INT 6B (0.83 g, 2.6 mmol, 49% yield) that was used without further purification. LCMS (m/z) calculated for C₇H₃BrClFN₂O₂S: 311.9; found 331.0 [M+H]⁺, t_{R}=3.67 min (Purity Method 4). ¹H NMR (400 MHz, DMSO) δ 9.40 (s, 1H), 7.71 (dd, J=8.7, 6.1 Hz, 1H), 7.21 (t, J=8.6 Hz, 1H).

### Step 6-3. Synthesis of 5-bromo-6-fluoro-3-(((3-fluoropyridin-2-yl)methyl)amino)-4H-benzo[e][1,2,4]thiadiazine 1,1-dioxide (INT 6C)

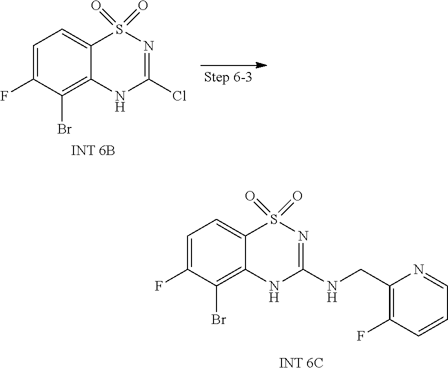 [SMILES: O=S1(=O)N=C(Cl)Nc2c1ccc(F)c2Br ; O=S1(=O)N=C(NCc2ncccc2F)Nc2c1ccc(F)c2Br]

To a stirring solution of 5-bromo-3-chloro-6-fluoro-4H-benzo[e][1,2,4]thiadiazine 1,1-dioxide INT 6B (100 mg, 1 Eq, 319 mol) in NMP (2 mL) was added (3-fluoropyridin-2-yl)methanamine (40.2 mg, 1 Eq, 319 mol). The reaction vial was capped and heated at 100° C. while stirring for 3 h. The reaction mixture was cooled to room temperature and purified by reversed phase HPLC (Gradient: 35-55% A; A-B mobile phase: [0.1% formic acid in MeCN-0.1% formic acid in H₂O]) to afford 5-bromo-6-fluoro-3-(((3-fluoropyridin-2-yl)methyl)amino)-4H-benzo[e][1,2,4]thiadiazine 1,1-dioxide INT 6C (38 mg, 94 mol, 30% yield). LCMS (m/z) calculated for C₁₃H₉BrF₂N₄O₂S: 402.0; found 402.2 [M+H]⁺, t_{R}=9.391 min (Purity Method 3). ¹H NMR (400 MHz, DMSO) δ 10.30 (s, 1H), 8.64 (s, 1H), 8.47 (d, J=4.7 Hz, 1H), 7.79 (t, J=9.1 Hz, 2H), 7.49 (dt, J=8.6, 4.5 Hz, 1H), 7.31 (t, J=8.6 Hz, 1H), 4.70 (dd, J=5.0, 1.8 Hz, 2H).

### Step 6-4. Synthesis of 6-fluoro-3-(((3-fluoropyridin-2-yl)methyl)amino)-5-phenethyl-4H-benzo[e][1,2,4]thiadiazine 1,1-dioxide (Compound 6-1)

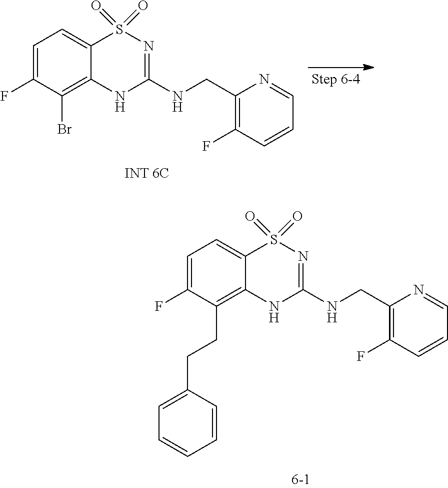 [SMILES: O=S1(=O)N=C(NCc2ncccc2F)Nc2c1ccc(F)c2Br ; O=S1(=O)N=C(NCc2ncccc2F)Nc2c1ccc(F)c2CCc1ccccc1]

To a solution of 5-bromo-6-fluoro-3-(((3-fluoropyridin-2-yl)methyl)amino)-4H-benzo[e][1,2,4]thiadiazine 1,1-dioxide INT 6C (15 mg, 1 Eq, 37 mol) in 1,4-dioxane (2 mL) and water (2 mL) was added cesium carbonate (24 mg, 2 Eq, 74 mol), phenethylboronic acid (17 mg, 3 Eq, 0.11 mmol), Pd(dppf)Cl₂ (2.7 mg, 0.1 Eq, 3.7 mol). The mixture was degassed and heated at 90° C. for 3 h while stirring. The reaction mixture was cooled to room temperature, passed through a syringe filter, and concentrated in vacuo to afford crude material. The crude material was purified by reversed phase HPLC (Gradient: 35-55% A; A-B mobile phase: [0.1% formic acid in MeCN-0.1% formic acid in H₂O]) to afford 6-fluoro-3-(((3-fluoropyridin-2-yl)methyl)amino)-5-phenethyl-4H-benzo[e][1,2,4]thiadiazine 1,1-dioxide 6-1 (0.7 mg, 2 μmol, 4% yield). LCMS (m/z) calculated for C₂₁H₁₈F₂N₄O₂S: 428.1; found 429.0 [M+H]⁺, t_{R}=8.66 min (Purity Method 3).

### Synthesis of Scheme 6 Compounds

Alternatively for Step 6-4, the following compounds were made using 2 Eq. of boronic acid, 3 Eq. of potassium phosphate tribasic, and 0.1 Eq. Pd(PPh₃)₄ at 110° C. for 14h: 6-2, 6-4, 6-5, 6-18.

The compounds listed in Table 7 were made using the procedures of Scheme 6.

TABLE 7

| | | Purity | Exact | | | |
|---|---|---|---|---|---|---|
| Cpd | | RT | mass | observed | | Purity |
| No. | Structure | (min) | (g/mol) | m/z | Ion | Method |
| 6-1 | 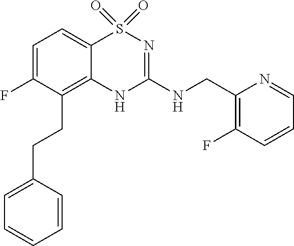 [SMILES: O=S1(=O)N=C(NCc2ncccc2F)Nc2c1ccc(F)c2CCc1ccccc1] | 8.696 | 428 | 429 | [M + H]⁺ | 3 |
| 6-2 | 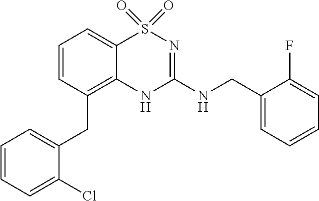 [SMILES: O=S1(=O)N=C(NCc2ccccc2F)Nc2c(Cc3ccccc3Cl)cccc21] | 8.867 | 429 | 430 | [M + H]⁺ | 3 |
| 6-3 | 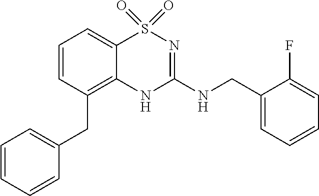 [SMILES: O=S1(=O)N=C(NCc2ccccc2F)Nc2c(Cc3ccccc3)cccc21] | 8.465 | 395 | 396 | [M + H]⁺ | 3 |
| 6-4 | 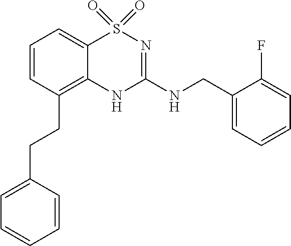 [SMILES: O=S1(=O)N=C(NCc2ccccc2F)Nc2c(CCc3ccccc3)cccc21] | 8.781 | 409 | 410 | [M + H]⁺ | 3 |
| 6-5 | 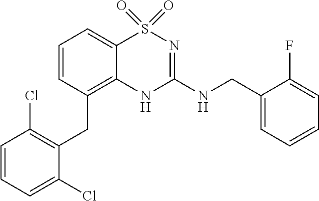 [SMILES: O=S1(=O)N=C(NCc2ccccc2F)Nc2c(Cc3c(Cl)cccc3Cl)cccc21] | 9.031 | 463 | 464 | [M + H]⁺ | 3 |
| 6-6 | 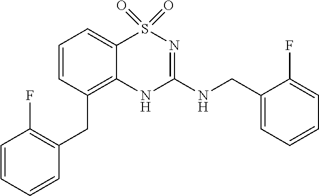 [SMILES: O=S1(=O)N=C(NCc2ccccc2F)Nc2c(Cc3ccccc3F)cccc21] | 8.437 | 413 | 414 | [M + H]⁺ | 3 |
| 6-7 | 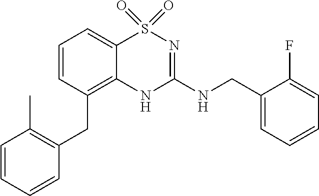 [SMILES: Cc1ccccc1Cc1cccc2c1NC(NCc1ccccc1F)=NS2(=O)=O] | 8.904 | 409 | 410 | [M + H]⁺ | 3 |
| 6-8 | 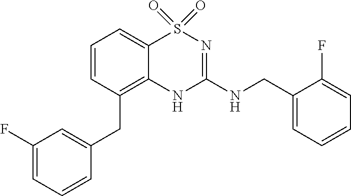 [SMILES: O=S1(=O)N=C(NCc2ccccc2F)Nc2c(Cc3cccc(F)c3)cccc21] | 8.472 | 413 | 414 | [M + H]⁺ | 3 |
| 6-9 | 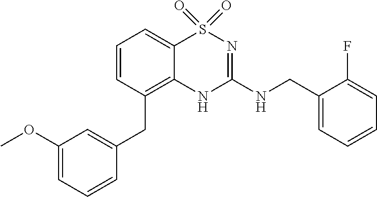 [SMILES: COc1cccc(Cc2cccc3c2NC(NCc2ccccc2F)=NS3(=O)=O)c1] | 8.378 | 425 | 426 | [M + H]⁺ | 3 |
| 6-10 | 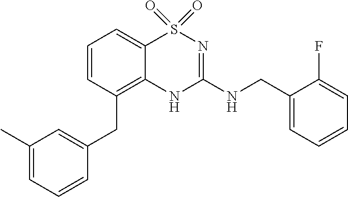 [SMILES: Cc1cccc(Cc2cccc3c2NC(NCc2ccccc2F)=NS3(=O)=O)c1] | 8.908 | 409 | 410 | [M + H]⁺ | 3 |
| 6-11 | 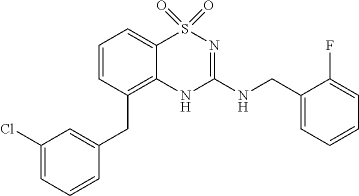 [SMILES: O=S1(=O)N=C(NCc2ccccc2F)Nc2c(Cc3cccc(Cl)c3)cccc21] | 8.947 | 429 | 430 | [M + H]⁺ | 3 |
| 6-12 | 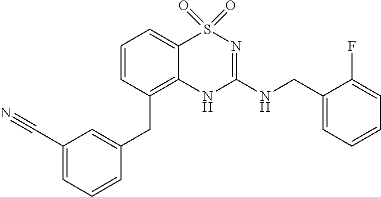 [SMILES: N#Cc1cccc(Cc2cccc3c2NC(NCc2ccccc2F)=NS3(=O)=O)c1] | 7.878 | 420 | 421 | [M + H]⁺ | 3 |
| 6-13 | 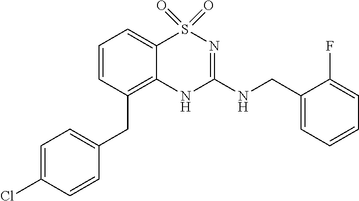 [SMILES: O=S1(=O)N=C(NCc2ccccc2F)Nc2c(Cc3ccc(Cl)cc3)cccc21] | 9.011 | 429 | 430 | [M + H]⁺ | 3 |
| 6-14 | 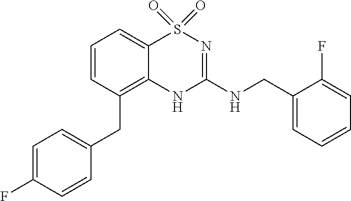 [SMILES: O=S1(=O)N=C(NCc2ccccc2F)Nc2c(Cc3ccc(F)cc3)cccc21] | 8.471 | 413 | 414 | [M + H]⁺ | 3 |
| 6-15 | 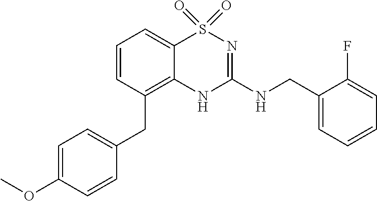 [SMILES: COc1ccc(Cc2cccc3c2NC(NCc2ccccc2F)=NS3(=O)=O)cc1] | 8.306 | 425 | 426 | [M + H]⁺ | 3 |
| 6-16 | 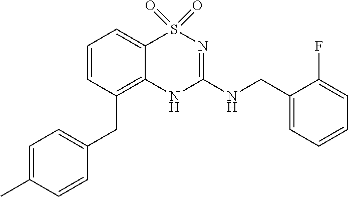 [SMILES: Cc1ccc(Cc2cccc3c2NC(NCc2ccccc2F)=NS3(=O)=O)cc1] | 8.915 | 409 | 410 | [M + H]⁺ | 3 |
| 6-17 | 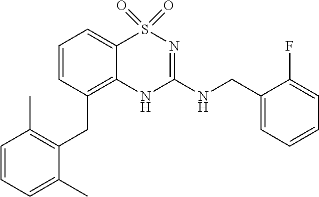 [SMILES: Cc1cccc(C)c1Cc1cccc2c1NC(NCc1ccccc1F)=NS2(=O)=O] | 9.362 | 423 | 424 | [M + H]⁺ | 3 |
| 6-18 | 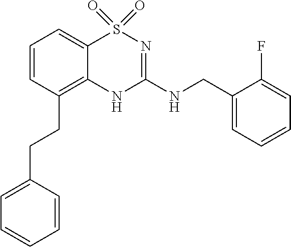 [SMILES: O=S1(=O)N=C(NCc2ccccc2F)Nc2c(CCc3ccccc3)cccc21] | 8.781 | 409 | 410 | [M + H]⁺ | 3 |
| 6-19 | 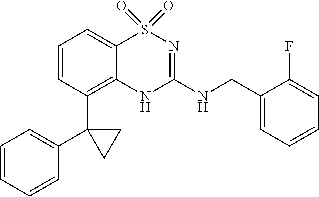 [SMILES: O=S1(=O)N=C(NCc2ccccc2F)Nc2c(C3(c4ccccc4)CC3)cccc21] | 8.852 | 421 | 422 | [M + H]⁺ | 3 |

### Example 7

### Synthesis of Representative Compound 7-7

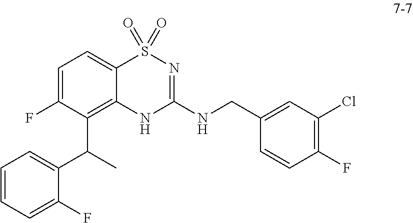 [SMILES: CC(c1ccccc1F)c1c(F)ccc2c1NC(NCc1ccc(F)c(Cl)c1)=NS2(=O)=O]

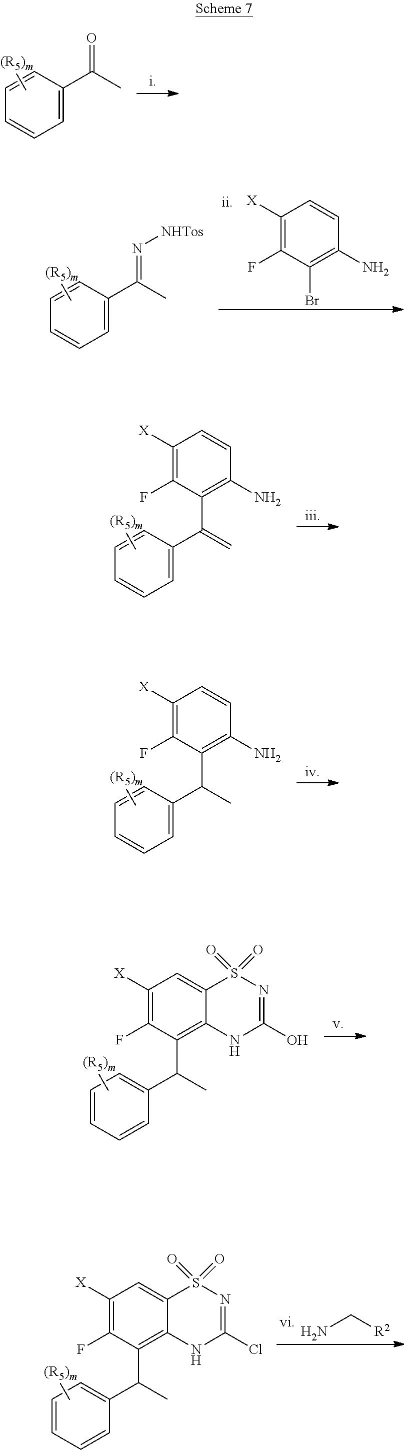

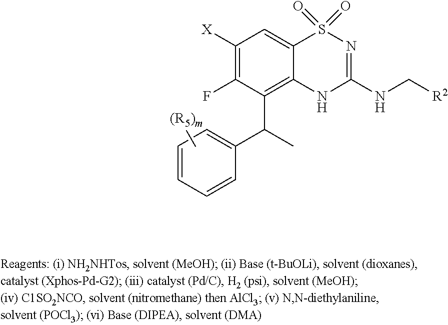

### Step 7-1. Synthesis of (E)-N′-(1-(2-fluorophenyl)ethylidene)-4-methylbenzenesulfonohydrazide-(INT 7A)

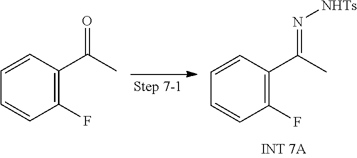

To a solution of 1-(2-fluorophenyl)ethanone (50.0 g, 362 mmol, 44.0 mL, 1 eq) in MeOH (500 mL), was added 4-methylbenzenesulfonohydrazide (68.1 g, 366 mmol, 1.01 eq). The mixture was heated and stirred at 60° C. for 14 h. The reaction mixture was concentrated in vacuo. The residue was triturated with MeOH (100 mL) and collected by filtration to afford (E)-N′-(1-(2-fluorophenyl)ethylidene)-4-methylbenzenesulfonohydrazide INT 7A (80 g, 261 mmol, 72% yield). ¹H NMR (400 MHz, DMSO-d6) δ=10.66 (s, 1H), 7.78 (d, J=8.3 Hz, 2H), 7.49-7.38 (m, 3H), 7.37-7.26 (m, 1H), 7.26-7.09 (m, 2H), 2.37 (s, 3H), 2.18 (d, J=2.5 Hz, 3H).

### Step 7-2. Synthesis of 3-fluoro-2-(1-(2-fluorophenyl)vinyl)aniline (INT 7B)

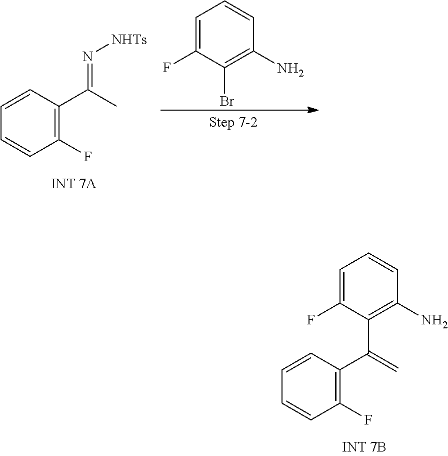

To a mixture of 2-bromo-3-fluoro-aniline (10 g, 52.6 mmol, 1 eq), N-[(E)-1-(2-fluorophenyl)ethylideneamino]-4-methyl-benzenesulfonamide INT 7A (20.0 g, 65.3 mmol, 1.24 eq) and t-BuOLi (8.50 g, 106 mmol, 9.57 mL, 2.02 eq) in dioxane (600 mL) was added XPhos-Pd-G2 (4.00 g, 5.08 mmol, 0.10 eq), then it was stirred at 100° C. for 15 h. The mixture was concentrated in vacuo. The residue was added to 200 mL of water. The aqueous layer was extracted with ethyl acetate (3×200 mL). The combined organic layer was dried over anhydrous sodium sulfate, filtered, and concentrated in vacuo. The residue was purified by silica gel chromatography (0 to 10% EtOAc/petroleum ether) to afford 3-fluoro-2-(1-(2-fluorophenyl)vinyl)aniline INT 7B (21.5 g, 87.4 mmol, 83% yield).

### Step 7-3. Synthesis of 3-fluoro-2-(1-(2-fluorophenyl)ethyl)aniline (INT 7C)

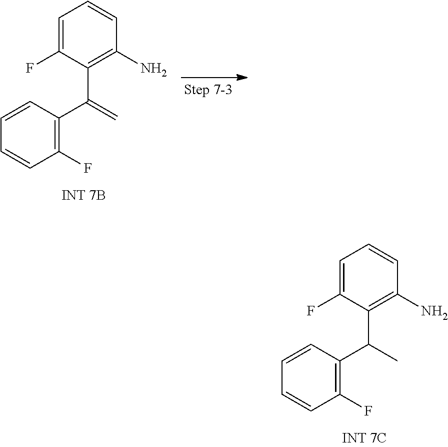 [SMILES: C=C(c1ccccc1F)c1c(N)cccc1F ; CC(c1ccccc1F)c1c(N)cccc1F]

A mixture of 3-fluoro-2-[1-(2-fluorophenyl)vinyl]aniline INT 7B (25 g, 108 mmol, 1 eq) and 10% Pd/C (2.5 g) in MeOH (300 mL) was degassed and purged with N₂ 3 times. The reaction mixture was stirred at 50° C. for 45 h under an H2 atmosphere (50 psi). The reaction mixture was filtered and concentrated in vacuo to afford 3-fluoro-2-(1-(2-fluorophenyl)ethyl)aniline INT 7C (16.9 g, 63.9 mmol, 70% yield) that was used without further purification. LCMS (m/z) calculated for C₁₄H₁₃F₂N: 233.1; found 234.2 [M+H]⁺, t_{R}=0.552 min (Purity Method 1).

### Step 7-4. Synthesis of 6-fluoro-5-(1-(2-fluorophenyl)ethyl)-3-hydroxy-4H-benzo[e][1,2,4]thiadiazine 1,1-dioxide (INT 7D)

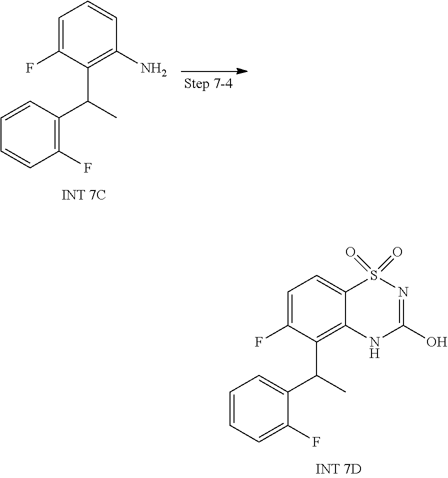 [SMILES: CC(c1ccccc1F)c1c(N)cccc1F ; CC(c1ccccc1F)c1c(F)ccc2c1NC(O)=NS2(=O)=O]

To a solution of 3-fluoro-2-[1-(2-fluorophenyl)ethyl]aniline INT 7C (4.0 g, 17.2 mmol, 1 eq) in Nitrosobenzene (60 mL) at 0° C., was added N-(oxomethylene)sulfamoyl chloride (3.59 g, 25.3 mmol, 2.20 mL, 1.48 eq). The mixture was stirred at 0° C. for 0.5 h. Then AlCl₃ (3.44 g, 25.8 mmol, 1.41 mL, 1.5 eq) was added. The mixture was heated and stirred at 60° C. for 1.5 h. The reaction solution was cooled to room temperature and then diluted with saturated NH₄Cl aqueous solution (300 mL). The aqueous phase was extracted with EtOAc (3×200 mL). The combined organic layers were washed with brine (100 mL), dried over anhydrous sodium sulfate, filtered and concentrated in vacuo. The crude oil was diluted with petroleum ether (600 mL) and then stirred for 30 min. The brown oil eventually formed a brown precipitate that was collected by vacuum filtration. The filter cake was washed with petroleum ether (200 mL) and dried under vacuum to give 6-fluoro-5-(1-(2-fluorophenyl)ethyl)-3-hydroxy-4H-benzo[e][1,2,4]thiadiazine 1,1-dioxide INT 7D 1 (4.5 g, 11.8 mmol, 69% yield) that was used without further purification. LCMS (m/z) calculated for C₁₅H₁₂F₂N₂O₃S: 338.1; found 337.0 [M−H], t_{R}=0.629 min (Purity Method 2).

### Step 7-5. Synthesis of 3-chloro-6-fluoro-5-(1-(2-fluorophenyl)ethyl)-4H-benzo[e][1,2,4]thiadiazine 1,1-dioxide (INT 7E)

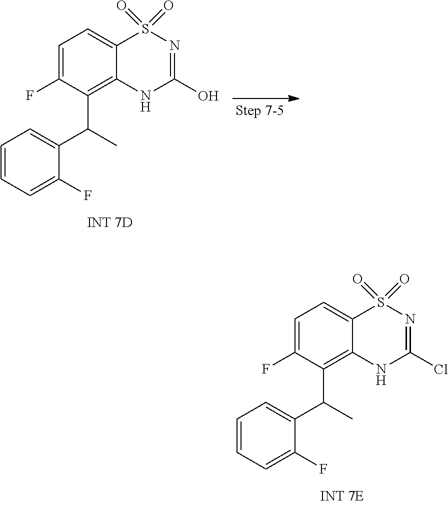 [SMILES: CC(c1ccccc1F)c1c(F)ccc2c1NC(O)=NS2(=O)=O ; CC(c1ccccc1F)c1c(F)ccc2c1NC(Cl)=NS2(=O)=O]

To a solution of 6-fluoro-5-[1-(2-fluorophenyl)ethyl]-1,1-dioxo-4H-1,2,4-benzothiadiazin-3-ol INT 7D (1.5 g, 4.4 mmol, 1 eq) in POCl₃ (10 mL), was added N,N-diethylaniline (1.03 g, 6.88 mmol, 1.10 mL, 1.55 eq). The reaction mixture was heated and stirred at 120° C. for 16 h. The reaction mixture was cooled to room temperature and poured into water (100 mL) portionwise while the temperature was kept below 35° C. The obtained mixture was stirred for 1 hr. The mixture was extracted with EtOAc (3×60 mL). The combined organic phase was washed with brine (2×60 mL), dried over Na₂SO₄, filtered, and concentrated in vacuo to give a residue. The aqueous mixture was adjusted to pH=7 by KOH and then discarded. The crude residue was purified by silica gel chromatography (0 to 65% EtOAc/petroleum ether) to afford 3-chloro-6-fluoro-5-(1-(2-fluorophenyl)ethyl)-4H-benzo[e][1,2,4]thiadiazine 1,1-dioxide (INT 7E) (1.1 g, 2.6 mmol, 59% yield) that was used without further purification. LCMS (m/z) calculated for C₁₅H₁₁ClF₂N₂O₂S: 356.0; found 355.0 [M−H], t_{R}=0.538 min (Purity Method 2).

### Step 7-6. Synthesis of 3-((3-chloro-4-fluorobenzyl)amino)-6-fluoro-5-(1-(2-fluorophenyl)ethyl)-4H-benzo[e][1,2,4]thiadiazine 1,1-dioxide (Compound 7-7)

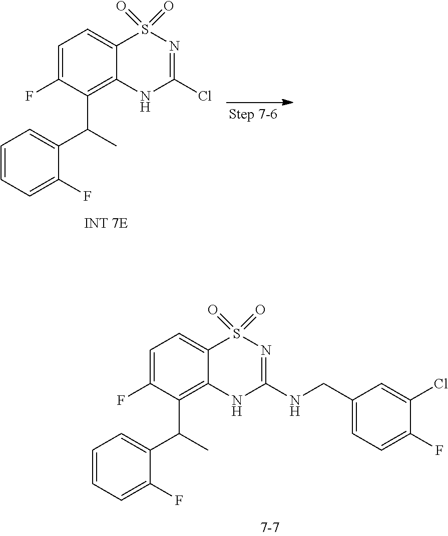 [SMILES: CC(c1ccccc1F)c1c(F)ccc2c1NC(Cl)=NS2(=O)=O ; CC(c1ccccc1F)c1c(F)ccc2c1NC(NCc1ccc(F)c(Cl)c1)=NS2(=O)=O]

To a stirring solution of (3-chloro-4-fluoro-phenyl)methanamine (34 mg, 210 mol, 1.5 eq) in DMA (1 mL) was added 3-chloro-6-fluoro-5-[1-(2-fluorophenyl)ethyl]-4H-1,2,4-benzothiadiazine 1,1-dioxide INT 7E (50 mg, 140 mol, 1 eq) and DIPEA (54 mg, 420 mol, 73 L, 3 eq). The mixture was stirred at 120° C. for 2 h. The crude mixture was filtered and concentrated in vacuo. The crude residue was purified by preparative HPLC (column: Welch Xtimate C18 150×25 mm×5 um; gradient: 43%-73% MeCN/0.1% formic acid in water over 10 min) and lyophilized to afford 3-((3-chloro-4-fluorobenzyl)amino)-6-fluoro-5-(1-(2-fluorophenyl)ethyl)-4H-benzo[e][1,2,4]thiadiazine 1,1-dioxide 7-7 (13.7 mg, 28.3 mol, 20% yield). LCMS (m/z) calculated for C₂₂H₁₇ClF₃N₃O₂S: 479.1; found 480.1 [M+H]⁺, t_{R}=0.540 min (Purity Method 1). ¹H NMR (400 MHz, DMSO-d6) δ=10.33 (s, 1H), 7.75 (br t, J=5.2 Hz, 1H), 7.68-7.59 (m, 2H), 7.55 (br t, J=7.7 Hz, 1H), 7.49-7.37 (m, 2H), 7.35-7.27 (m, 1H), 7.27-7.21 (m, 1H), 7.09 (dd, J=8.3, 10.0 Hz, 1H), 7.01 (dd, J=8.9, 11.1 Hz, 1H), 4.65 (q, J=6.7 Hz, 1H), 4.59-4.44 (m, 2H), 1.69 (d, J=7.1 Hz, 3H).

### Synthesis of Scheme 7 Compounds

Alternatively for step 7-3, the following compounds were made using 0.2 Eq of tris(triphenylphosphine)rhodium(I) chloride in THE with H2 (50 psi) at a reaction temperature of 80° C. for 72 h: 7-2.

Alternatively for step 7-3, the following compounds were made using 0.05 Eq of PtO₂ in MeOH with H2 (1 atm) at room temperature for 24 h: 7-1.

The compounds listed in Table 8 were made using the procedures of Scheme 7.

TABLE 8

| | | Purity | Exact | | | |
|---|---|---|---|---|---|---|
| Cpd | | RT | mass | observed | | Purity |
| No. | Structure | (min) | (g/mol) | m/z | Ion | Method |
| 7-1 | 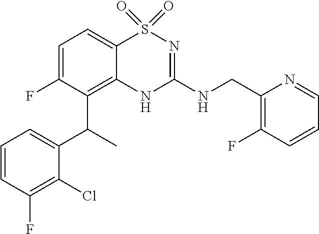 [SMILES: CC(c1cccc(F)c1Cl)c1c(F)ccc2c1NC(NCc1ncccc1F)=NS2(=O)=O] | 0.514 | 480.1 | 481.3 | [M + H]⁺ | 1 |
| 7-2 | 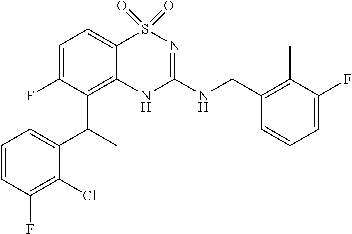 [SMILES: Cc1c(F)cccc1CNC1=NS(=O)(=O)c2ccc(F)c(C(C)c3cccc(F)c3Cl)c2N1] | 0.554 | 493.1 | 494 | [M + H]⁺ | 1 |
| 7-3 | 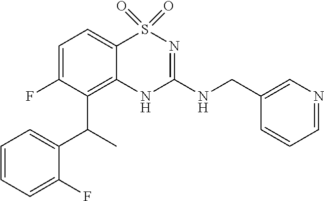 [SMILES: CC(c1ccccc1F)c1c(F)ccc2c1NC(NCc1cccnc1)=NS2(=O)=O] | 0.412 | 428.1 | 429.1 | [M + H]⁺ | 1 |
| 7-4 | 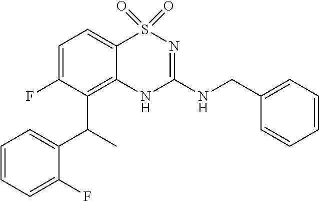 [SMILES: CC(c1ccccc1F)c1c(F)ccc2c1NC(NCc1ccccc1)=NS2(=O)=O] | 0.521 | 427.1 | 428.1 | [M + H]⁺ | 1 |
| 7-5 | 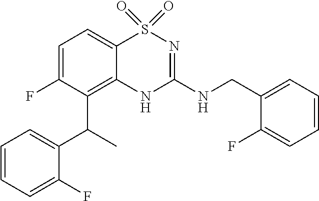 [SMILES: CC(c1ccccc1F)c1c(F)ccc2c1NC(NCc1ccccc1F)=NS2(=O)=O] | 0.529 | 445.1 | 446.1 | [M + H]⁺ | 1 |
| 7-6 | 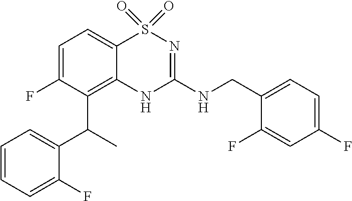 [SMILES: CC(c1ccccc1F)c1c(F)ccc2c1NC(NCc1ccc(F)cc1F)=NS2(=O)=O] | 0.531 | 463.1 | 464.1 | [M + H]⁺ | 1 |
| 7-7 | 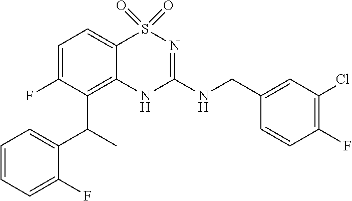 [SMILES: CC(c1ccccc1F)c1c(F)ccc2c1NC(NCc1ccc(F)c(Cl)c1)=NS2(=O)=O] | 0.540 | 479.1 | 480.1 | [M + H]⁺ | 1 |
| 7-8 | 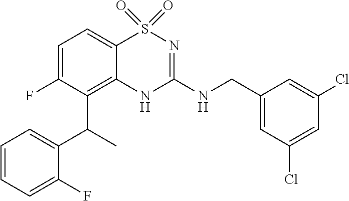 [SMILES: CC(c1ccccc1F)c1c(F)ccc2c1NC(NCc1cc(Cl)cc(Cl)c1)=NS2(=O)=O] | 0.562 | 495.0 | 496 | [M + H]⁺ | 3 |
| 7-9 | 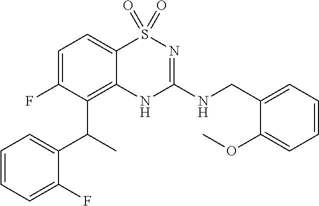 [SMILES: COc1ccccc1CNC1=NS(=O)(=O)c2ccc(F)c(C(C)c3ccccc3F)c2N1] | 0.530 | 457.1 | 458.1 | [M + H]⁺ | 1 |
| 7-10 | 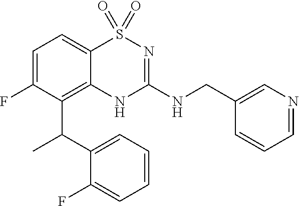 [SMILES: CC(c1ccccc1F)c1c(F)ccc2c1NC(NCc1cccnc1)=NS2(=O)=O] | 0.412 | 428.1 | 429.1 | [M + H]⁺ | 1 |
| 7-11 | 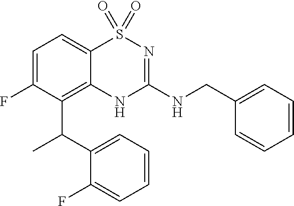 [SMILES: CC(c1ccccc1F)c1c(F)ccc2c1NC(NCc1ccccc1)=NS2(=O)=O] | 0.521 | 427.1 | 428.1 | [M + H]⁺ | 1 |
| 7-12 | 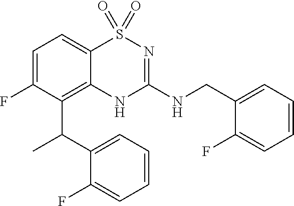 [SMILES: CC(c1ccccc1F)c1c(F)ccc2c1NC(NCc1ccccc1F)=NS2(=O)=O] | 0.529 | 445.1 | 446.1 | [M + H]⁺ | 1 |
| 7-13 | 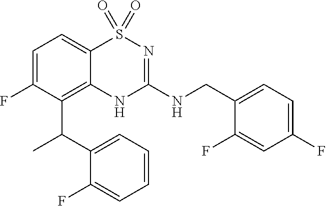 [SMILES: CC(c1ccccc1F)c1c(F)ccc2c1NC(NCc1ccc(F)cc1F)=NS2(=O)=O] | 0.531 | 463.1 | 464.1 | [M + H]⁺ | 1 |
| 7-14 | 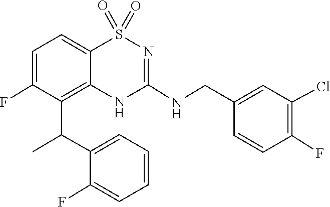 [SMILES: CC(c1ccccc1F)c1c(F)ccc2c1NC(NCc1ccc(F)c(Cl)c1)=NS2(=O)=O] | 0.540 | 479.1 | 480.1 | [M + H]⁺ | 1 |
| 7-15 | 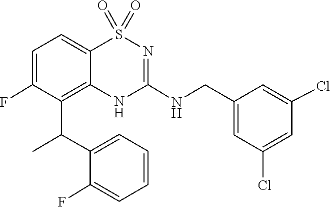 [SMILES: CC(c1ccccc1F)c1c(F)ccc2c1NC(NCc1cc(Cl)cc(Cl)c1)=NS2(=O)=O] | 10.006 | 495.0 | 496.0 | [M + H]⁺ | 3 |
| 7-16 | 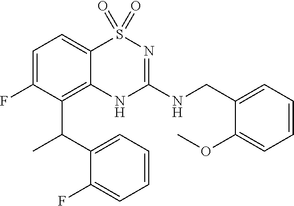 [SMILES: COc1ccccc1CNC1=NS(=O)(=O)c2ccc(F)c(C(C)c3ccccc3F)c2N1] | 0.530 | 457.1 | 458.1 | [M + H]⁺ | 1 |
| 7-17 | 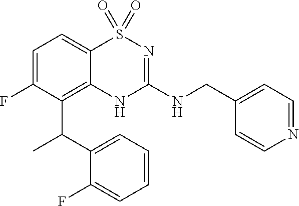 [SMILES: CC(c1ccccc1F)c1c(F)ccc2c1NC(NCc1ccncc1)=NS2(=O)=O] | 0.430 | 428.1 | 429.1 | [M + H]⁺ | 1 |
| 7-18 | 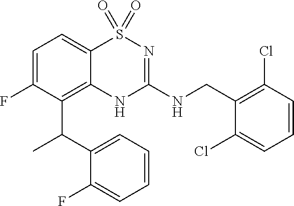 [SMILES: CC(c1ccccc1F)c1c(F)ccc2c1NC(NCc1c(Cl)cccc1Cl)=NS2(=O)=O] | 0.549 | 495.0 | 496.0 | [M + H]⁺ | 1 |
| 7-19 | 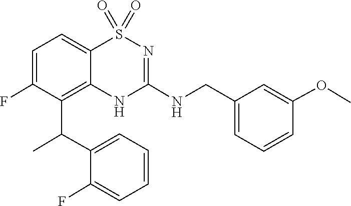 [SMILES: COc1cccc(CNC2=NS(=O)(=O)c3ccc(F)c(C(C)c4ccccc4F)c3N2)c1] | 0.523 | 457.1 | 458.1 | [M + H]⁺ | 1 |
| 7-20 | 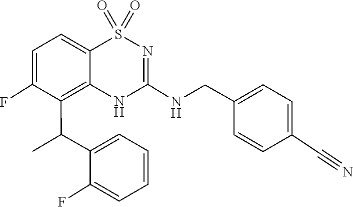 [SMILES: CC(c1ccccc1F)c1c(F)ccc2c1NC(NCc1ccc(C#N)cc1)=NS2(=O)=O] | 0.503 | 452.1 | 453.0 | [M + H]⁺ | 1 |
| 7-21 | 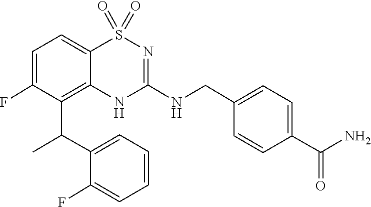 [SMILES: CC(c1ccccc1F)c1c(F)ccc2c1NC(NCc1ccc(C(N)=O)cc1)=NS2(=O)=O] | 0.459 | 470.1 | 471.1 | [M + H]⁺ | 1 |
| 7-22 | 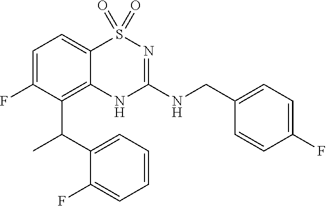 [SMILES: CC(c1ccccc1F)c1c(F)ccc2c1NC(NCc1ccc(F)cc1)=NS2(=O)=O] | 0.525 | 445.1 | 446.0 | [M + H]⁺ | 1 |
| 7-23 | 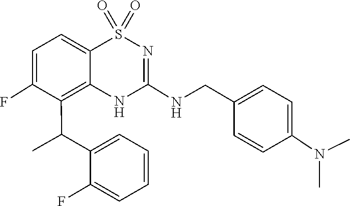 [SMILES: CC(c1ccccc1F)c1c(F)ccc2c1NC(NCc1ccc(N(C)C)cc1)=NS2(=O)=O] | 0.445 | 470.2 | 471.0 | [M + H]⁺ | 1 |
| 7-24 | 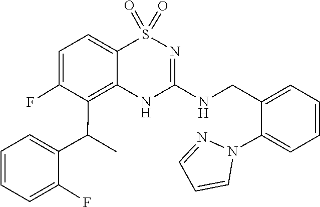 [SMILES: CC(c1ccccc1F)c1c(F)ccc2c1NC(NCc1ccccc1-n1cccn1)=NS2(=O)=O] | 0.520 | 493.1 | 494.1 | [M + H]⁺ | 1 |
| 7-25 | 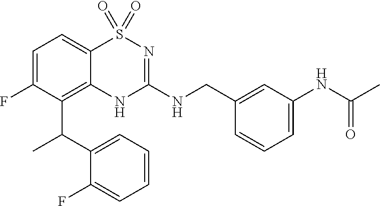 [SMILES: CC(=O)Nc1cccc(CNC2=NS(=O)(=O)c3ccc(F)c(C(C)c4ccccc4F)c3N2)c1] | 0.482 | 484.1 | 485.1 | [M + H]⁺ | 1 |
| 7-26 | 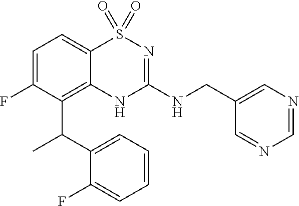 [SMILES: CC(c1ccccc1F)c1c(F)ccc2c1NC(NCc1cncnc1)=NS2(=O)=O] | 0.465 | 429.1 | 430.1 | [M + H]⁺ | 1 |
| 7-27 | 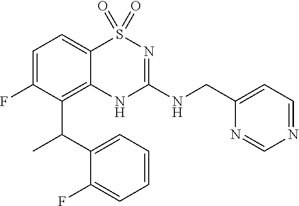 [SMILES: CC(c1ccccc1F)c1c(F)ccc2c1NC(NCc1ccncn1)=NS2(=O)=O] | 0.472 | 429.1 | 430.0 | [M + H]⁺ | 1 |
| 7-28 | 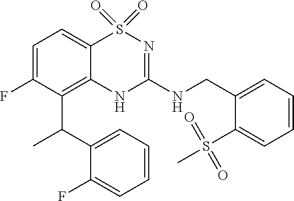 [SMILES: CC(c1ccccc1F)c1c(F)ccc2c1NC(NCc1ccccc1S(C)(=O)=O)=NS2(=O)=O] | 0.518 | 505.1 | 505.9 | [M + H]⁺ | 1 |
| 7-29 | 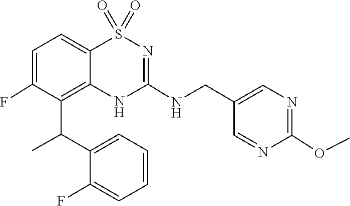 [SMILES: COc1ncc(CNC2=NS(=O)(=O)c3ccc(F)c(C(C)c4ccccc4F)c3N2)cn1] | 0.486 | 459.1 | 460.0 | [M + H]⁺ | 1 |
| 7-30 | 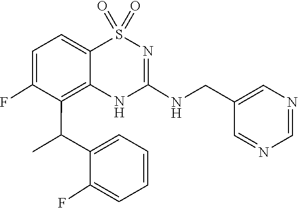 [SMILES: CC(c1ccccc1F)c1c(F)ccc2c1NC(NCc1cncnc1)=NS2(=O)=O] | 0.466 | 429.1 | 430.0 | [M + H]⁺ | 1 |
| 7-31 | 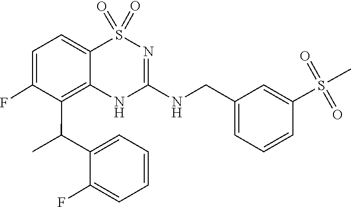 [SMILES: CC(c1ccccc1F)c1c(F)ccc2c1NC(NCc1cccc(S(C)(=O)=O)c1)=NS2(=O)=O] | 0.499 | 505.1 | 505.9 | [M + H]⁺ | 1 |
| 7-32 | 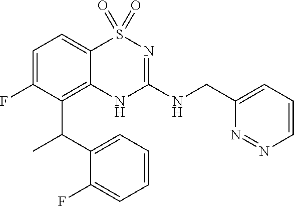 [SMILES: CC(c1ccccc1F)c1c(F)ccc2c1NC(NCc1cccnn1)=NS2(=O)=O] | 0.461 | 429.1 | 430.1 | [M + H]⁺ | 1 |
| 7-33 | 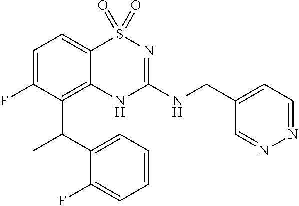 [SMILES: CC(c1ccccc1F)c1c(F)ccc2c1NC(NCc1ccnnc1)=NS2(=O)=O] | 0.461 | 429.1 | 430.2 | [M + H]⁺ | 1 |
| 7-34 | 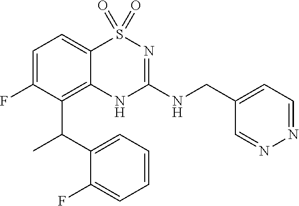 | 0.500 | 505.1 | 506.0 | [M + H]⁺ | 1 |
| 7-35 | 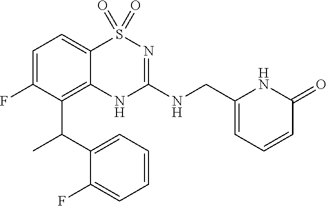 [SMILES: CC(c1ccccc1F)c1c(F)ccc2c1NC(NCc1cccc(=O)[nH]1)=NS2(=O)=O] | 0.461 | 444.1 | 445.0 | [M + H]⁺ | 1 |
| 7-36 | 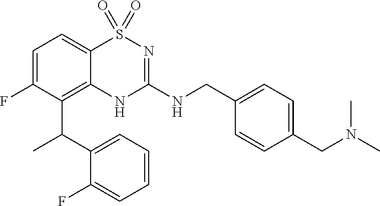 [SMILES: CC(c1ccccc1F)c1c(F)ccc2c1NC(NCc1ccc(CN(C)C)cc1)=NS2(=O)=O] | 0.453 | 484.2 | 485.1 | [M + H]⁺ | 1 |
| 7-37 | 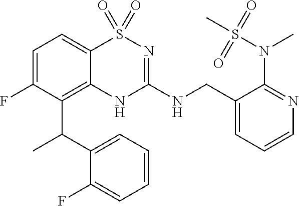 [SMILES: CC(c1ccccc1F)c1c(F)ccc2c1NC(NCc1cccnc1N(C)S(C)(=O)=O)=NS2(=O)=O] | 0.503 | 535.1 | 536.1 | [M + H]⁺ | 1 |
| 7-38 | 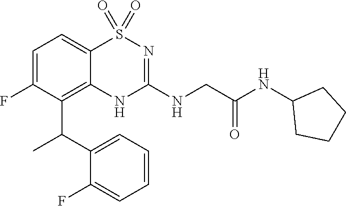 [SMILES: CC(c1ccccc1F)c1c(F)ccc2c1NC(NCC(=O)NC1CCCC1)=NS2(=O)=O] | 0.510 | 462.2 | 463.1 | [M + H]⁺ | 1 |
| 7-39 | 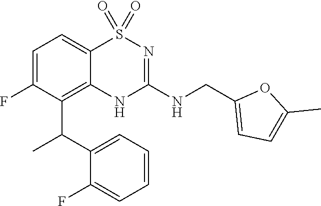 [SMILES: Cc1ccc(CNC2=NS(=O)(=O)c3ccc(F)c(C(C)c4ccccc4F)c3N2)o1] | 0.536 | 431.1 | 432.1 | [M + H]⁺ | 1 |
| 7-40 | 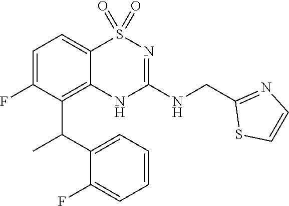 [SMILES: CC(c1ccccc1F)c1c(F)ccc2c1NC(NCc1nccs1)=NS2(=O)=O] | 0.493 | 434.1 | 435.0 | [M + H]⁺ | 1 |
| 7-41 | 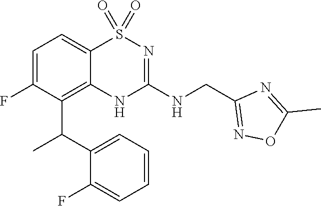 [SMILES: Cc1nc(CNC2=NS(=O)(=O)c3ccc(F)c(C(C)c4ccccc4F)c3N2)no1] | 0.482 | 433.1 | 434.0 | [M + H]⁺ | 1 |
| 7-42 | 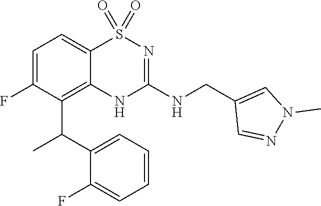 [SMILES: CC(c1ccccc1F)c1c(F)ccc2c1NC(NCc1cnn(C)c1)=NS2(=O)=O] | 0.482 | 431.1 | 432.1 | [M + H]⁺ | 1 |
| 7-43 | 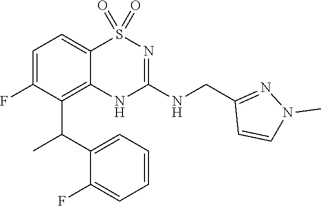 [SMILES: CC(c1ccccc1F)c1c(F)ccc2c1NC(NCc1ccn(C)n1)=NS2(=O)=O] | 0.486 | 431.1 | 432.0 | [M + H]⁺ | 1 |
| 7-44 | 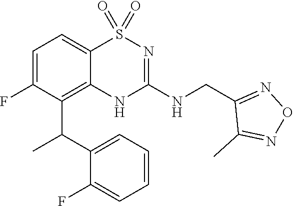 [SMILES: Cc1nonc1CNC1=NS(=O)(=O)c2ccc(F)c(C(C)c3ccccc3F)c2N1] | 0.504 | 433.1 | 434.0 | [M + H]⁺ | 1 |
| 7-45 | 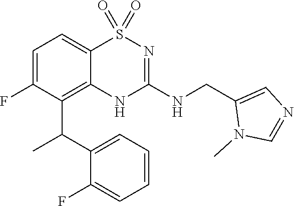 [SMILES: CC(c1ccccc1F)c1c(F)ccc2c1NC(NCc1cncn1C)=NS2(=O)=O] | 0.434 | 431.1 | 432.0 | [M + H]⁺ | 1 |
| 7-46 | 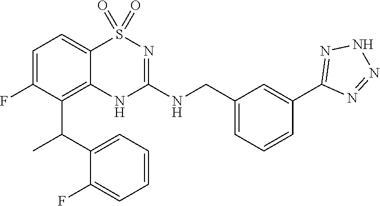 [SMILES: CC(c1ccccc1F)c1c(F)ccc2c1NC(NCc1cccc(-c3nn[nH]n3)c1)=NS2(=O)=O] | 0.493 | 495.1 | 496.1 | [M + H]⁺ | 1 |
| 7-47 | 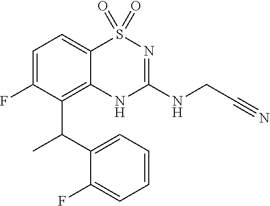 [SMILES: CC(c1ccccc1F)c1c(F)ccc2c1NC(NCC#N)=NS2(=O)=O] | 0.484 | 376.1 | 377.0 | [M + H]⁺ | 1 |
| 7-48 | 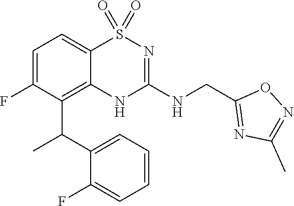 [SMILES: Cc1noc(CNC2=NS(=O)(=O)c3ccc(F)c(C(C)c4ccccc4F)c3N2)n1] | 0.487 | 433.1 | 434.0 | [M + H]⁺ | 1 |
| 7-49 | 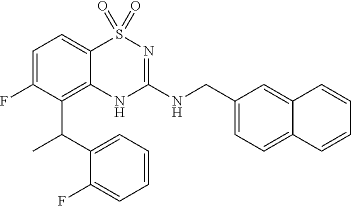 [SMILES: CC(c1ccccc1F)c1c(F)ccc2c1NC(NCc1ccc3ccccc3c1)=NS2(=O)=O] | 0.56 | 477.1 | 478.1 | [M + H]⁺ | 1 |
| 7-50 | 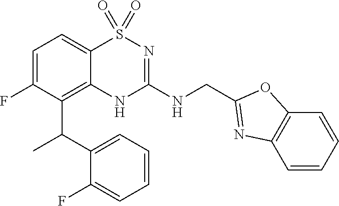 [SMILES: CC(c1ccccc1F)c1c(F)ccc2c1NC(NCc1nc3ccccc3o1)=NS2(=O)=O] | 0.519 | 468.1 | 469.0 | [M + H]⁺ | 1 |
| 7-51 | 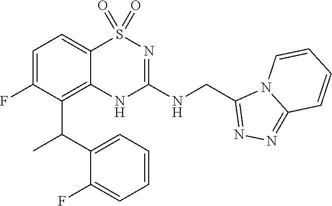 [SMILES: CC(c1ccccc1F)c1c(F)ccc2c1NC(NCc1nnc3ccccn13)=NS2(=O)=O] | 0.461 | 468.1 | 469.1 | [M + H]⁺ | 1 |
| 7-52 | 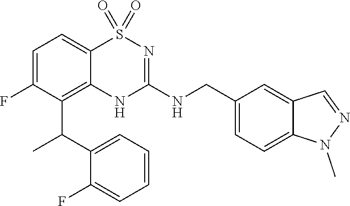 [SMILES: CC(c1ccccc1F)c1c(F)ccc2c1NC(NCc1ccc3c(cnn3C)c1)=NS2(=O)=O] | 0.511 | 481.1 | 482.1 | [M + H]⁺ | 1 |
| 7-53 | 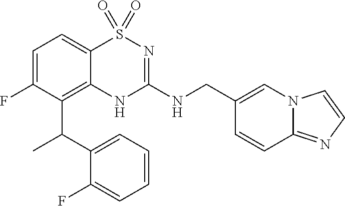 [SMILES: CC(c1ccccc1F)c1c(F)ccc2c1NC(NCc1ccc3nccn3c1)=NS2(=O)=O] | 0.449 | 467.1 | 468.1 | [M + H]⁺ | 1 |
| 7-54 | 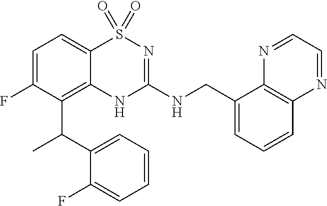 [SMILES: CC(c1ccccc1F)c1c(F)ccc2c1NC(NCc1cccc3nccnc13)=NS2(=O)=O] | 0.514 | 479.1 | 480.1 | [M + H]⁺ | 1 |
| 7-55 | 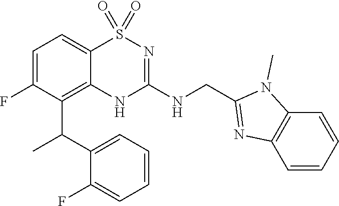 [SMILES: CC(c1ccccc1F)c1c(F)ccc2c1NC(NCc1nc3ccccc3n1C)=NS2(=O)=O] | 0.469 | 481.1 | 482.1 | [M + H]⁺ | 1 |
| 7-56 | 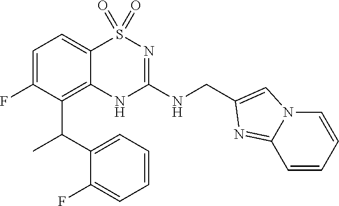 [SMILES: CC(c1ccccc1F)c1c(F)ccc2c1NC(NCc1cn3ccccc3n1)=NS2(=O)=O] | 0.457 | 467.1 | 468.1 | [M + H]⁺ | 1 |
| 7-57 | 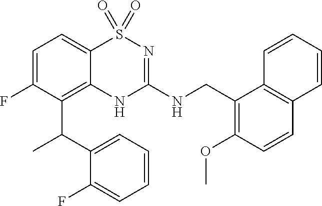 [SMILES: COc1ccc2ccccc2c1CNC1=NS(=O)(=O)c2ccc(F)c(C(C)c3ccccc3F)c2N1] | 0.57 | 507.1 | 508.1 | [M + H]⁺ | 1 |
| 7-58 | 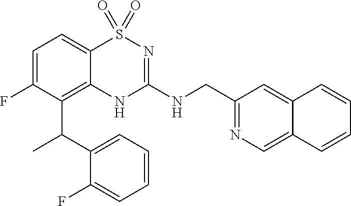 [SMILES: CC(c1ccccc1F)c1c(F)ccc2c1NC(NCc1cc3ccccc3cn1)=NS2(=O)=O] | 0.495 | 478.1 | 479.1 | [M + H]⁺ | 1 |

### Example 8

### Synthesis of Representative Compound 8-3

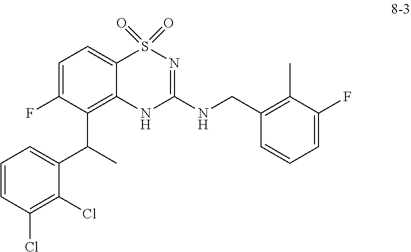 [SMILES: Cc1c(F)cccc1CNC1=NS(=O)(=O)c2ccc(F)c(C(C)c3cccc(Cl)c3Cl)c2N1]

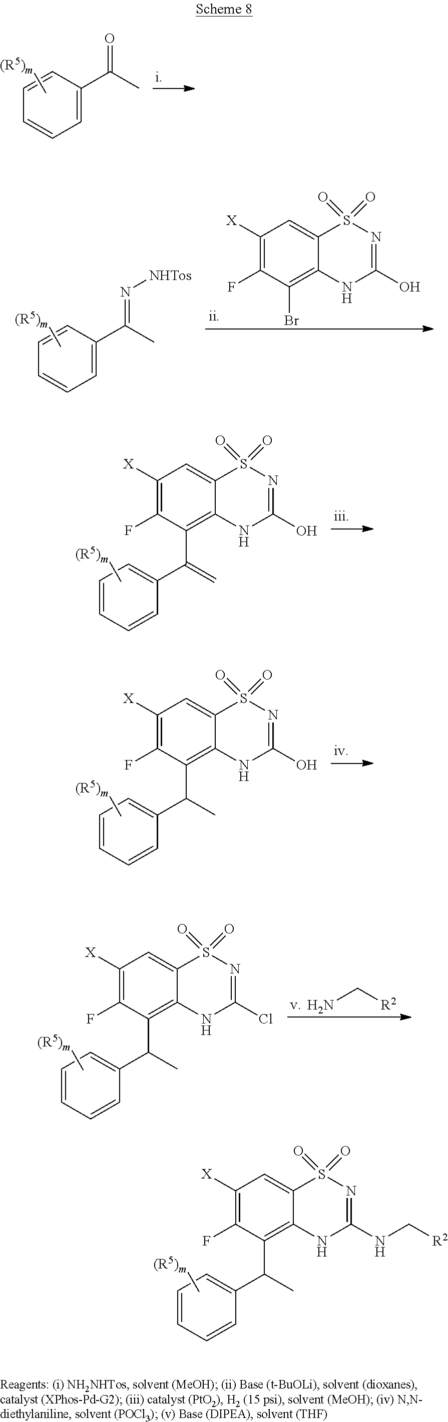

### Step 8-1. Synthesis (E)-N′-(1-(2,3-dichlorophenyl)ethylidene)-4-methylbenzenesulfonohydrazide (INT 8A)

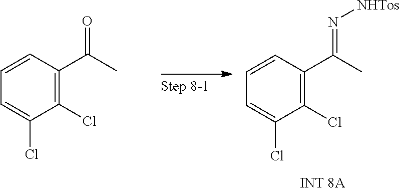

To a solution of 1-(2,3-dichlorophenyl)ethanone (2.0 g, 10.6 mmol, 1 eq) in MeOH (20 mL) was added 4-methylbenzenesulfonohydrazide (2.17 g, 11.6 mmol, 1.1 eq). The reaction mixture was heated and stirred at 60° C. for 15 h. The reaction mixture was cooled to room temperature and the resultant precipitate was filtered. The precipitate was washed with MeOH and dried under high vacuum to afford (E)-N′-(1-(2,3-dichlorophenyl)ethylidene)-4-methylbenzenesulfonohydrazide INT 8A (3.3 g, 9.13 mmol, 86% yield). LCMS (m/z) calculated for C₁₅H₁₄Cl₂N₂O₂S: 355.0; found 480.1 [M−H], t_{R}=0.443 min (Purity Method 1). ¹H NMR (400 MHz, DMSO-d6) δ=10.77 (s, 1H), 7.74 (d, J=8.3 Hz, 2H), 7.63 (dd, J=1.5, 8.1 Hz, 1H), 7.43-7.31 (m, 3H), 7.15 (dd, J=1.5, 7.6 Hz, 1H), 2.37 (s, 3H), 2.16 (s, 3H).

### Step 8-2. Synthesis 5-(1-(2,3-dichlorophenyl)vinyl)-6-fluoro-3-hydroxy-4H-benzo[e][1,2,4]thiadiazine 1,1-dioxide (INT 8B)

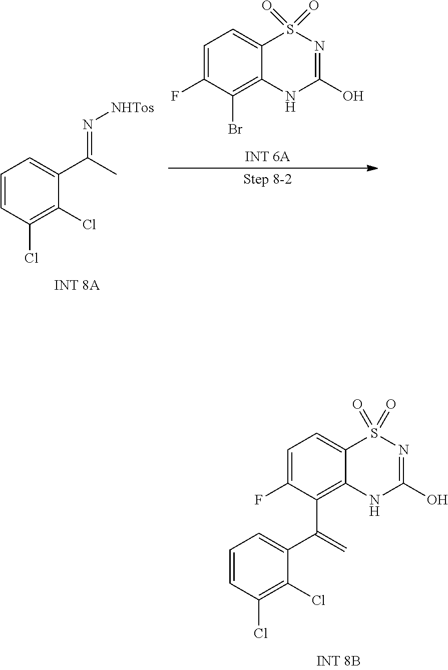

To a solution of N-[(E)-1-(2,3-dichlorophenyl)ethylideneamino]-4-methyl-benzenesulfonamide INT 8A (1.54 g, 4.32 mmol, 1.5 eq) and 5-bromo-6-fluoro-1,1-dioxo-4H-1,2,4-benzothiadiazin-3-ol INT 6A (850 mg, 2.88 mmol, 1 eq) in dioxane (16 mL) was added lithium, 2-methylpropan-2-olate (692 mg, 8.64 mmol, 779 μL, 3 eq) and XPhos-Pd-G2 (227 mg, 288 mol, 0.1 eq). The reaction mixture was heated and stirred at 100° C. for 15 h. After returning to room temperature, water (15 mL) was added to the reaction mixture. The aqueous layer was extracted with EtOAc (3×15 mL). The aqueous phase was adjusted to pH=5 by diluted hydrochloric acid and extracted with ethyl acetate (3×15 mL). The combined organics were washed with aqueous saturated brine solution (2×10 mL), dried over anhydrous sodium sulfate, filtered, and concentrated in vacuo to give a crude residue. The residue was purified by prep-HPLC (column: Waters Xbridge Prep OBD C18 150×40 mm×10 um; A-B mobile phase: [water(NH₄HCO₃)-ACN]; gradient: 10%-40% B over 15 min) to afford 5-(1-(2,3-dichlorophenyl)vinyl)-6-fluoro-3-hydroxy-4H-benzo[e][1,2,4]thiadiazine 1,1-dioxide INT 8B (180 mg, 440 mol, 15% yield). LCMS (m/z) calculated for C₁₅H₉Cl₂FN₂O₃S: 386.0; found 384.9 [M−H], t_{R}=0.518 min (Purity Method 2).

### Step 8-3. Synthesis 5-(1-(2,3-dichlorophenyl)ethyl)-6-fluoro-3-hydroxy-4H-benzo[e][1,2,4]thiadiazine 1,1-dioxide (INT 8C)

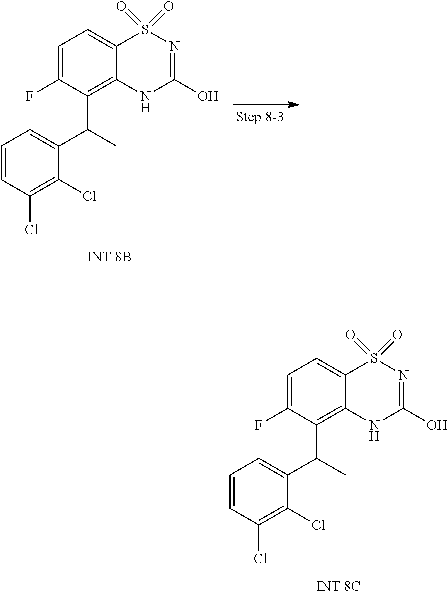 [SMILES: CC(c1cccc(Cl)c1Cl)c1c(F)ccc2c1NC(O)=NS2(=O)=O ; CC(c1cccc(Cl)c1Cl)c1c(F)ccc2c1NC(O)=NS2(=O)=O]

A solution of 5-[1-(2,3-dichlorophenyl)vinyl]-6-fluoro-1,1-dioxo-4H-1,2,4-benzothiadiazin-3-ol INT 8B (340 mg, 878 mol, 1 eq) in MeOH (20 mL) was degassed and purged with N₂ 3 times. PtO₂ (200 mg) was added and degassed and purged with N₂ 3 times. The reaction mixture was then put under an H2 atmosphere (15 psi) and stirred for 4 h at room temperature. The reaction mixture was concentrated in vacuo to give 5-(1-(2,3-dichlorophenyl)ethyl)-6-fluoro-3-hydroxy-4H-benzo[e][1,2,4]thiadiazine 1,1-dioxide INT 8C that was used without further purification. LCMS (m/z) calculated for C₁₅H₁₁Cl₂FN₂O₃S: 388.0; found 386.8 [M−H], t_{R}=0.514 min (Purity Method 2).

### Step 8-4. Synthesis 3-chloro-5-(1-(2,3-dichlorophenyl)ethyl)-6-fluoro-4H-benzo[e][1,2,4]thiadiazine 1,1-dioxide (INT 8D)

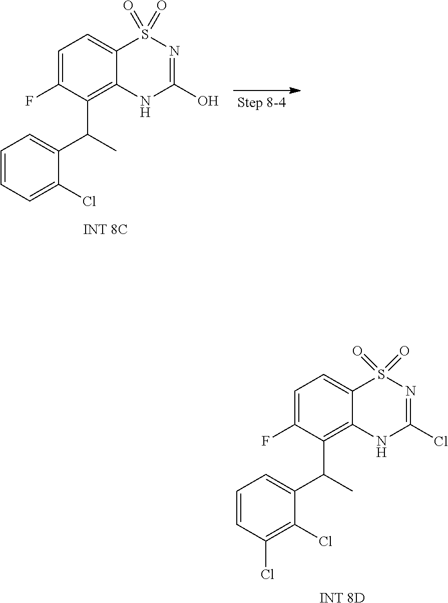 [SMILES: CC(c1ccccc1Cl)c1c(F)ccc2c1NC(O)=NS2(=O)=O ; CC(c1cccc(Cl)c1Cl)c1c(F)ccc2c1NC(Cl)=NS2(=O)=O ; "INT 8D"]

To a solution of 5-[1-(2,3-dichlorophenyl)ethyl]-6-fluoro-1,1-dioxo-4H-1,2,4-benzothiadiazin-3-ol INT 8C (280 mg, 719 mol, 1 eq) in POCl₃ (2 mL) was added N,N-diethylaniline (161 mg, 1.08 mmol, 173 μL, 1.5 eq). The mixture was heated and stirred at 120° C. for 2 h. The reaction mixture was cooled to room temperature and quenched by the addition of sodium bicarbonate solution (30 mL). The aqueous layer was extracted with EtOAc (3×30 mL), and the combined organic layer was washed with brine (2×20 mL), filtered, and concentrated in vacuo to give a crude residue. The crude residue was purified by silica gel chromatography (0á100% EtOAc/petroleum ether) to afford 3-chloro-5-(1-(2,3-dichlorophenyl)ethyl)-6-fluoro-4H-benzo[e][1,2,4]thiadiazine 1,1-dioxide INT 8D (220 mg, 453 mol, 63% yield) that was used without further purification. LCMS (m/z) calculated for C₁₅H₁₀Cl₃FN₂O₂S: 406.0; found 404.9 [M−H], t_{R}=0.587 min (Purity Method 2).

### Step 8-5. Synthesis of 5-(1-(2,3-dichlorophenyl)ethyl)-6-fluoro-3-((3-fluoro-2-methylbenzyl)amino)-4H-benzo[e][1,2,4]thiadiazine 1,1-dioxide (Compound 8-3)

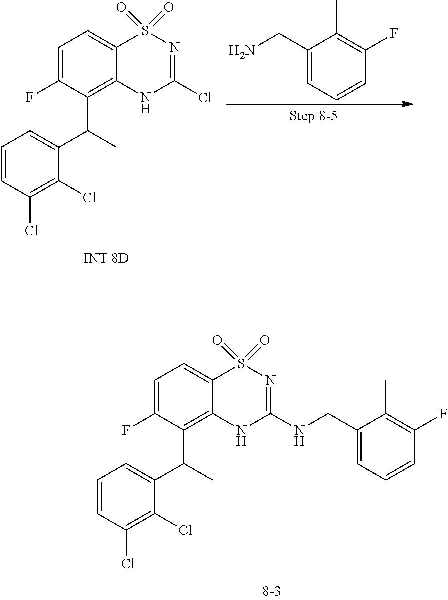

To a solution of 3-chloro-5-[1-(2,3-dichlorophenyl)ethyl]-6-fluoro-4H-1,2,4-benzothiadiazine 1,1-dioxide INT 8D (220 mg, 540 mol, 1 eq) and (3-fluoro-2-methyl-phenyl)methanamine (350 mg, 2.51 mmol, 4.66 eq) in THF (3 mL) was added DIPEA (209 mg, 1.62 mmol, 282 μL, 3 eq). The mixture was heated and stirred at 60° C. for 15 h. The reaction mixture was concentrated in vacuo to give a crude residue. The crude residue was purified by prep-HPLC (column: Welch Xtimate C18 150×25 mm×5 um; A-B mobile phase: [water(FA)-ACN]; gradient: 50%-70% B over 10 min) to afford 5-(1-(2,3-dichlorophenyl)ethyl)-6-fluoro-3-((3-fluoro-2-methylbenzyl)amino)-4H-benzo[e][1,2,4]thiadiazine 1,1-dioxide 8-3 (63.2 mg, 123.8 mol, 23% yield). LCMS (m/z) calculated for C₂₃H₁₉Cl₂F₂N₃O₂S: 509.0; found 510.0 [M+H]⁺, t_{R}=0.571 min (Purity Method 1). ¹H NMR (400 MHz, DMSO-d6) δ=10.35 (s, 1H), 7.75-7.61 (m, 3H), 7.58 (d, J=7.9 Hz, 1H), 7.50-7.42 (m, 1H), 7.32-7.22 (m, 1H), 7.21-7.17 (m, 1H), 7.16-7.09 (m, 1H), 7.01-6.91 (m, 1H), 4.63-4.46 (m, 3H), 2.26 (s, 3H), 1.68 (br d, J=7.0 Hz, 3H).

### Synthesis of Scheme 8 Compounds

Alternatively for step 8-2, the following compounds were made using 3 Eq of lithium; 2-methylpropan-2-olate in dioxanes with 0.1 Eq Pd(PPh₃)₂Cl₂ at a reaction temperature of 100° C. for 15 h: 8-1.

The compounds listed in Table 9 were made using the procedures of Scheme 8.

TABLE 9

| | | Purity | Exact | | | |
|---|---|---|---|---|---|---|
| Cpd | | RT | mass | observed | | Purity |
| No. | Structure | (min) | (g/mol) | m/z | Ion | Method |
| 8-1 | 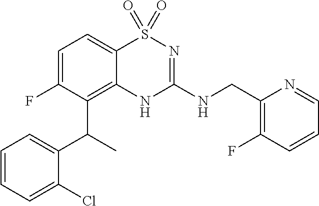 | 0.571 | 509.1 | 510 | [M + H]⁺ | 1 |
| 8-2 | 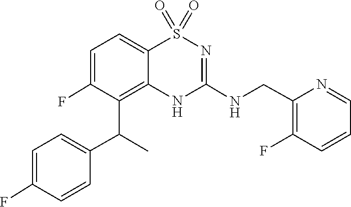 [SMILES: CC(c1ccc(F)cc1)c1c(F)ccc2c1NC(NCc1ncccc1F)=NS2(=O)=O] | 0.531 | 446.1 | 447.1 | [M + H]⁺ | 1 |
| 8-3 | 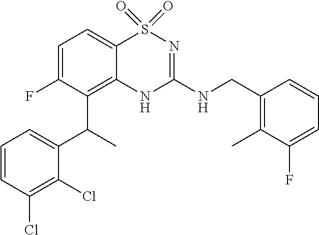 | 0.521 | 462.1 | 463.2 | [M + H]⁺ | 1 |

### Example 9

### Synthesis of Representative Compound 9-7

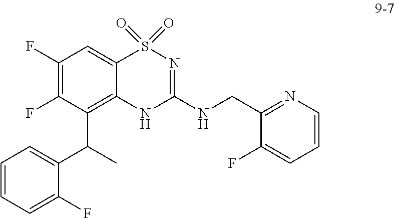 [SMILES: CC(c1ccccc1F)c1c(F)c(F)cc2c1NC(NCc1ncccc1F)=NS2(=O)=O]

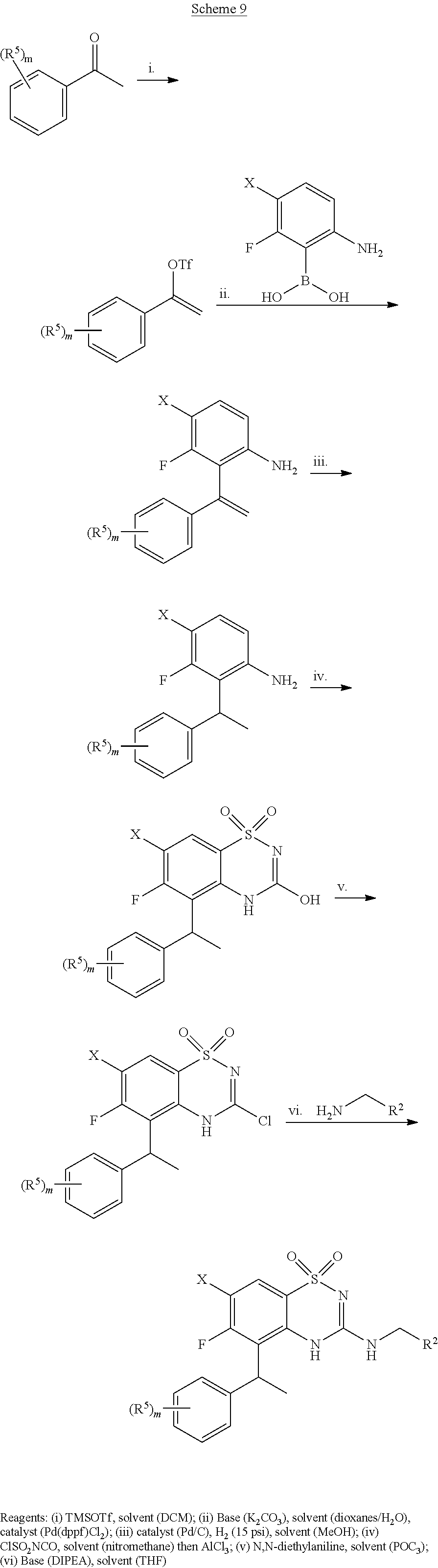

### Synthesis of (6-amino-2,3-difluorophenyl)boronic acid INT 9

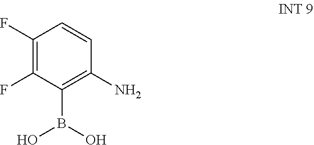 [SMILES: Nc1ccc(F)c(F)c1B(O)O]

To a solution of 2-bromo-3,4-difluoro-aniline (5.0 g, 24 mmol, 1 eq) and 4,4,5,5-tetramethyl-2-(4,4,5,5-tetramethyl1,3,2-dioxaborolan-2-yl)-1,3,2-dioxaborolane (12.2 g, 48.1 mmol, 2 eq) in dioxane (100 mL) was added KOAc (7.08 g, 72.1 mmol, 3 eq) and XphosPdG4 (1.03 g, 1.20 mmol, 0.05 eq). The reaction mixture was heated and stirred at 80° C. for 15 h. The reaction mixture was filtered and concentrated in vacuo to afford (6-amino-2,3-difluorophenyl)boronic acid INT 9 (4.0 g) that was used without further purification.

### Step 9-1. Synthesis of 1-(2-fluorophenyl)vinyl trifluoromethanesulfonate (INT 9A)

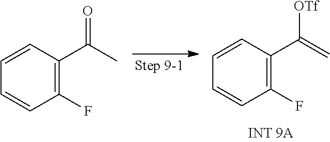

To a solution of 1-(2-fluorophenyl)ethanone (5.0 g, 36.2 mmol, 4.4 mL, 1 eq) in DCM (100 mL) at 0° C. was added 2,6-ditert-butyl-4-methyl-pyridine (8.18 g, 39.8 mmol, 1.1 eq) and trifluoromethylsulfonyl trifluoromethanesulfonate (12.25 g, 43.43 mmol, 7.17 mL, 1.2 eq). The mixture was stirred at 25° C. for 16 h. The reaction mixture was concentrated in vacuo. Petroleum ether was added and solid pyridinium triflate was filtered off and washed with petroleum ether. The combined petroleum ether solution was washed subsequently with cool HCl (1 M) and saturated brine. The combined organics were dried over anhydrous sodium sulfate, filtered, and concentrated in vacuo to afford 1-(2-fluorophenyl)vinyl trifluoromethanesulfonate INT 9A (10 g) that was used without further purification.

### Step 9-2. Synthesis of 3,4-difluoro-2-(1-(2-fluorophenyl)vinyl)aniline (INT 9B)

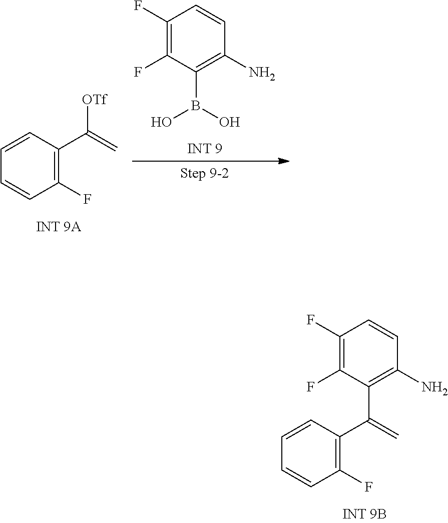 [SMILES: C=C(OS(=O)(=O)C(F)(F)F)c1ccccc1F ; Nc1ccc(F)c(F)c1B(O)O ; C=C(c1ccccc1F)c1c(N)ccc(F)c1F]

To a solution of 1-(2-fluorophenyl)vinyl trifluoromethanesulfonate INT 9A (3.8 g, 14.1 mmol, 1 eq) and (6-amino-2,3-difluoro-phenyl)boronic acid INT 9 (4.0 g, 23 mmol, 1.5 eq) in dioxane (100 mL) and H₂O (20 mL) was added K₂CO₃ (6.39 g, 46.3 mmol, 3 eq) and Pd(dppf)Cl2 (1.13 g, 1.54 mmol, 0.1 eq). The reaction mixture was heated and stirred at 80° C. for 4 h. The reaction mixture was cooled to room temperature and concentrated in vacuo. To the crude residue was added aqueous sodium bicarbonate solution (100 mL). The aqueous layer was extracted with EtOAc (3×100 mL). The organic layer was washed with brine solution (2×50 mL), dried over anhydrous sodium sulfate, filtered, and concentrated in vacuo to give a crude residue. The crude residue was purified by silica gel chromatography (0 to 20% EtOAc/petroleum ether) to afford 3,4-difluoro-2-(1-(2-fluorophenyl)vinyl)aniline INT 9B (1.0 g, 3.39 mmol, 22% yield) that was used without further purification. LCMS (m/z) calculated for C₁₄H₁₀F₃N: 249.1; found 250.1 [M+H]⁺, t_{R}=0.558 min (Purity Method 1).

### Step 9-3. Synthesis of 3,4-difluoro-2-(1-(2-fluorophenyl)ethyl)aniline INT 9C)

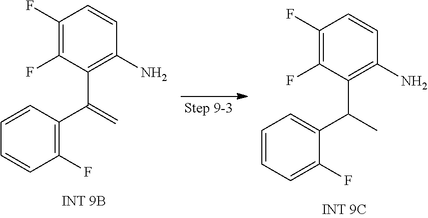 [SMILES: C=C(c1ccccc1F)c1c(N)ccc(F)c1F ; CC(c1ccccc1F)c1c(N)ccc(F)c1F]

A mixture of 3,4-difluoro-2-[1-(2-fluorophenyl)vinyl]aniline INT 9B (1.0 g, 4.0 mmol, 1 eq) and 10% Pd/C (4.27 g, 4.01 mmol, 1.00 eq) in MeOH (50 mL) was degassed and purged with N₂ 3 times. The reaction mixture was then stirred at 25° C. for 4 h under an H2 atmosphere (15 psi). The reaction mixture was filtered and concentrated in vacuo to afford 3,4-difluoro-2-(1-(2-fluorophenyl)ethyl)aniline INT 9C (700 mg, 2.33 mmol, 72% yield) that was used without further purification. LCMS (m/z) calculated for C₁₄H₁₂F₃N: 251.1; found 252.2 [M+H]⁺, t_{R}=0.560 min (Purity Method 1).

### Step 9-4. Synthesis of 6,7-difluoro-5-(1-(2-fluorophenyl)ethyl)-3-hydroxy-4H-benzo[e][1,2,4]thiadiazine 1,1-dioxide (INT 9D)

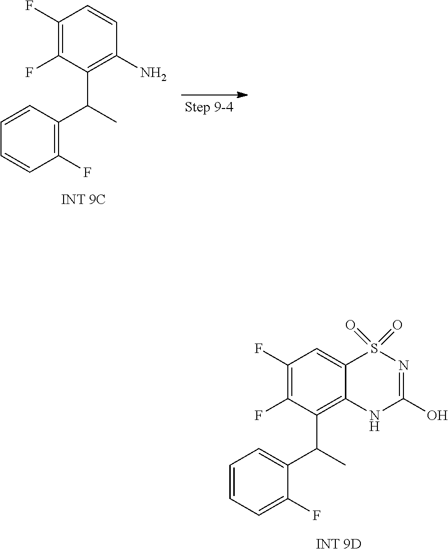 [SMILES: CC(c1ccccc1F)c1c(N)ccc(F)c1F ; CC(c1ccccc1F)c1c(F)c(F)cc2c1NC(O)=NS2(=O)=O]

To a solution of N-(oxomethylene)sulfamoyl chloride (552 mg, 3.90 mmol, 340 μL, 1.4 eq) in nitrobenzene (3 mL) at 0° C. was added a solution of 3,4-difluoro-2-[1-(2-fluorophenyl)ethyl]aniline (700 mg, 2.79 mmol, 1 eq) in nitrobenzene (1 mL). The reaction mixture was stirred at 0° C. for 0.5 h. Then, AlCl₃ (557 mg, 4.18 mmol, 1.5 eq) was added. The reaction mixture was heated and stirred at 120° C. for 1.5 h. The reaction mixture was cooled to room temperature and quenched by addition of ammonium chloride aqueous solution (10 mL). The aqueous layer was extracted with EtOAc (3×10 mL). The combined organics were concentrated in vacuo to afford a crude residue. The crude residue was washed with petroleum ether (2×10 mL) and filtered to give 6,7-difluoro-5-(1-(2-fluorophenyl)ethyl)-3-hydroxy-4H-benzo[e][1,2,4]thiadiazine 1,1-dioxide INT 9D (790 mg) that was used without further purification. LCMS (m/z) calculated for C₁₅H₁₁F₃N₂O₃S: 356.0; found 355.1 [M−H], t_{R}=0.445 min (Purity Method 2).

### Step 9-5. Synthesis of 3-chloro-6,7-difluoro-5-(1-(2-fluorophenyl)ethyl)-4H-benzo[e][1,2,4]thiadiazine 1,1-dioxide (INT 9E)

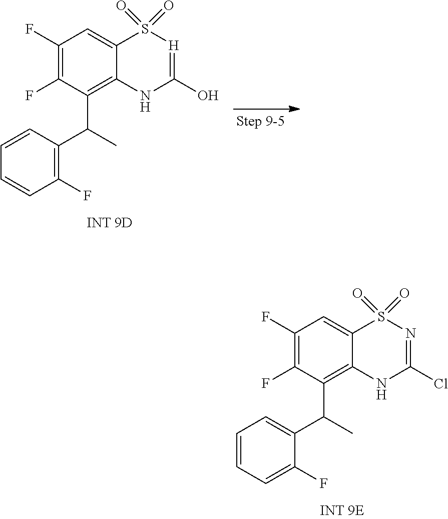

To a solution of 6,7-difluoro-5-[1-(2-fluorophenyl)ethyl]-1,1-dioxo-4H-1,2,4-benzothiadiazin-3-ol INT 9D (400 mg, 1.12 mmol, 1 eq) in POCl₃ (5 mL) was added N,N-diethylaniline (251 mg, 1.68 mmol, 269 μL, 1.5 eq). The reaction mixture was heated and stirred at 120° C. for 2 h. The reaction mixture was cooled to room temperature and quenched by the addition of an aqueous sodium bicarbonate solution (30 mL). The aqueous layer was extracted with EtOAc (3×30 mL). The combined organics were washed with brine (2×20 mL), dried over sodium sulfate, filtered, and concentrated in vacuo to give a residue. The residue was purified by silica gel chromatography (0á100% EtOAc/petroleum ether) to afford 3-chloro-6,7-difluoro-5-(1-(2-fluorophenyl)ethyl)-4H-benzo[e][1,2,4]thiadiazine 1,1-dioxide INT 9E (230 mg, 487 mol, 43% yield) that was used without further purification. LCMS (m/z) calculated for C₁₅H₁₀ClF₃N₂O₂S: 374.0; found 373.0 [M−H], t_{R}=0.528 min (Purity Method 2)

### Step 9-6. Synthesis of 6,7-difluoro-5-(1-(2-fluorophenyl)ethyl)-3-(((3-fluoropyridin-2-yl)methyl)amino)-4H-benzo[e][1,2,4]thiadiazine 1,1-dioxide (Compound 9-7)

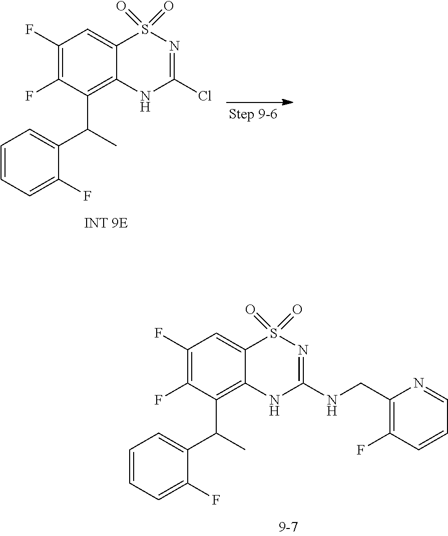 [SMILES: CC(c1ccccc1F)c1c(F)c(F)cc2c1NC(Cl)=NS2(=O)=O ; CC(c1ccccc1F)c1c(F)c(F)cc2c1NC(NCc1ncccc1F)=NS2(=O)=O]

To a solution of 3-chloro-6,7-difluoro-5-[1-(2-fluorophenyl)ethyl]-4H-1,2,4-benzothiadiazine 1,1-dioxide INT 9E (230 mg, 614 mol, 1 eq) and (3-fluoro-2-pyridyl)methanamine; dihydrochloride (244 mg, 1.23 mmol, 2 eq) in THE (3 mL) was added DIPEA (397 mg, 3.07 mmol, 534 μL, 5 eq). The mixture was heated and stirred at 60° C. for 15 h. The reaction mixture was concentrated under reduced pressure to give a crude residue. The residue was purified by prep-HPLC (column: Waters Xbridge 150×25 mm×5 um; A-B mobile phase: [water(NH₄HCO₃)-ACN]; gradient: 32%-62% B over 9 m) to afford 6,7-difluoro-5-(1-(2-fluorophenyl)ethyl)-3-(((3-fluoropyridin-2-yl)methyl)amino)-4H-benzo[e][1,2,4]thiadiazine 1,1-dioxide 9-7 (33.6 mg, 71.3 μmol, 12% yield). LCMS (m/z) calculated for C₂₁H₁₆F₄N₄O₂S: 464.1; found 465.1 [M−H], t_{R}=0.533 mi (Purity Method 1). ¹H NMR (400 MHz, DMSO-d6) δ=10.60 (br s, 1H), 8.47 (br d, J=3.4 Hz, 1H), 8.12 (br s, 1H), 7.80 (t, J=9.3 Hz, 1H), 7.73 (br t, J=8.3 Hz, 1H), 7.60 (br t, J=7.6 Hz, 1H), 7.50 (td, J=4.4, 8.4 Hz, 1H), 7.3 8-7.3 0 (m, 1H), 7.30-7.23 (m, 1H), 7.17-7.08 (m, 1H), 4.79 (br d, J=5.9 Hz, 1H), 4.71 (br d, J=3.8 Hz, 2H), 1.72 (br d, J=7.0 Hz, 3H).

### Synthesis of Scheme 9 Compounds

The compounds listed in Table 10 were made using the procedures of Scheme 9.

TABLE 10

| | | Purity | Exact | | | |
|---|---|---|---|---|---|---|
| Cpd | | RT | mass | observed | | Purity |
| No. | Structure | (min) | (g/mol) | m/z | Ion | Method |
| 9-1 | 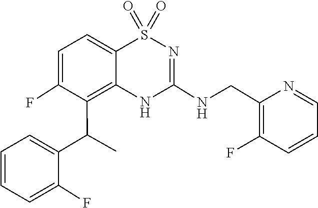 [SMILES: CC(c1ccccc1F)c1c(F)ccc2c1NC(NCc1ncccc1F)=NS2(=O)=O] | 0.509 | 446.1 | 447.3 | [M + H]⁺ | 1 |
| 9-2 | 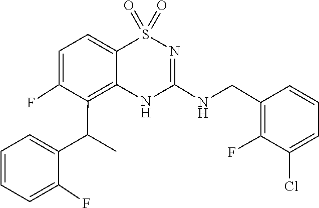 [SMILES: CC(c1ccccc1F)c1c(F)ccc2c1NC(NCc1cccc(Cl)c1F)=NS2(=O)=O] | 0.563 | 479.1 | 480.1 | [M + H]⁺ | 1 |
| 9-3 | 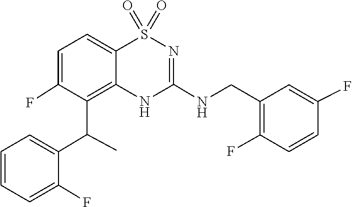 [SMILES: CC(c1ccccc1F)c1c(F)ccc2c1NC(NCc1cc(F)ccc1F)=NS2(=O)=O] | 0.548 | 463.1 | 464.1 | [M + H]⁺ | 1 |
| 9-4 | 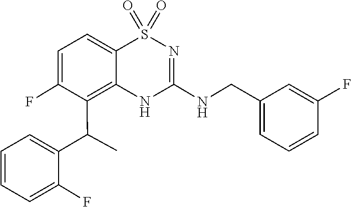 [SMILES: CC(c1ccccc1F)c1c(F)ccc2c1NC(NCc1cccc(F)c1)=NS2(=O)=O] | 0.548 | 445.1 | 446.1 | [M + H]⁺ | 1 |
| 9-5 | 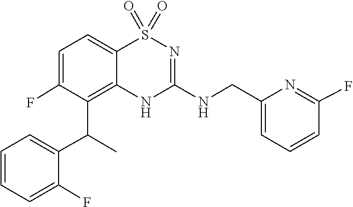 [SMILES: CC(c1ccccc1F)c1c(F)ccc2c1NC(NCc1cccc(F)n1)=NS2(=O)=O] | 0.522 | 446.1 | 447.1 | [M + H]⁺ | 1 |
| 9-6 | 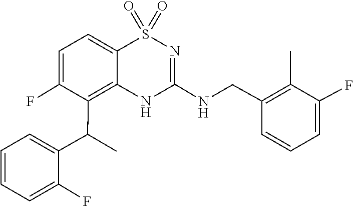 [SMILES: Cc1c(F)cccc1CNC1=NS(=O)(=O)c2ccc(F)c(C(C)c3ccccc3F)c2N1] | 0.562 | 459.1 | 460.1 | [M + H]⁺ | 1 |
| 9-7 | 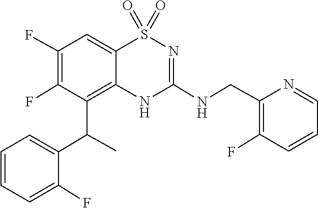 [SMILES: CC(c1ccccc1F)c1c(F)c(F)cc2c1NC(NCc1ncccc1F)=NS2(=O)=O] | 0.533 | 464.1 | 465.1 | [M + H]⁺ | 1 |

### Example 10

### Chiral Separation

### Chiral Separation of Compound 9-6 into Compound 10-3A and Compound 10-3B

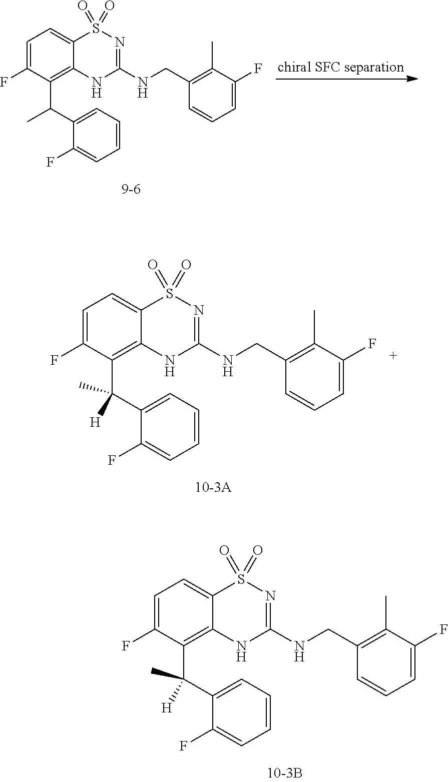 [SMILES: Cc1c(F)cccc1CNC1=NS(=O)(=O)c2ccc(F)c(C(C)c3ccccc3F)c2N1 ; Cc1c(F)cccc1CNC1=NS(=O)(=O)c2ccc(F)c([C@@H](C)c3ccccc3F)c2N1 ; Cc1c(F)cccc1CNC1=NS(=O)(=O)c2ccc(F)c([C@H](C)c3ccccc3F)c2N1]

SFC separation conditions of 9-6: (column: DAICEL CHIRALCEL OD (250 mm×30 mm, 10 μm); mobile phase: [CO₂-i-PrOH (0.1% NH₃H₂O)]; 40% i-PrOH (0.1% NH₃H₂O), isocratic elution mode) to afford 10-3A and 10-3B.

The above-mentioned chiral column was used for each chiral separation.

### Chiral Separation of Compound 9-1 into Compound 10-1A and Compound 10-1B

SFC separation conditions of 9-1: mobile phase: [CO₂-EtOH (0.1% NH₃H₂O)]; 35% EtOH (0.1% NH₃H₂O), isocratic elution mode) to afford 10-1A and 10-1B.

### Chiral Separation of Compound 9-5 into Compound 10-2A and Compound 10-2B

SFC separation conditions of 9-5: mobile phase: [CO₂-EtOH (0.1% NH₃H₂O)]; 30% EtOH (0.1% NH₃H₂O), isocratic elution mode) to afford 10-2A and 10-2B.

### Chiral Separation of Compound 8-2 into Compound 10-4A and Compound 10-4B

SFC separation conditions of 8-2: mobile phase: [CO₂-EtOH]; 25% EtOH (0.1% NH₃H₂O), isocratic elution mode) to afford 10-4A and 10-4B.

### Chiral Separation of Compound 9-4 into Compound 10-5A and Compound 10-5B

SFC separation conditions of 9-4: mobile phase: [CO₂-EtOH]; 35% EtOH (0.1% NH₃H₂O), isocratic elution mode) to afford 10-5A and 10-5B.

### Chiral Separation Table

The compounds listed in Table 11 were separated using the conditions set forth above.

TABLE 11

| | | Chiral | | | | | |
|---|---|---|---|---|---|---|---|
| | | SFC | Purity | Exact | | | |
| Cpd | | RT | RT | mass | observed | | Purity |
| No. | Structure | (min) | (min) | (g/mol) | m/z | Ion | Method |
| 10-1A | 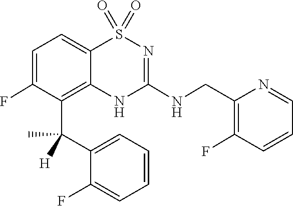 [SMILES: C[C@@H](c1ccccc1F)c1c(F)ccc2c1NC(NCc1ncccc1F)=NS2(=O)=O] | 1.611 | 0.518 | 446.1 | 447.1 | [M + H]⁺ | 1 |
| 10-1B | 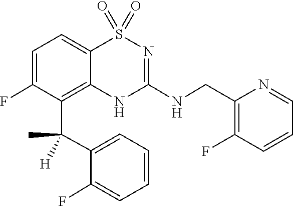 [SMILES: C[C@H](c1ccccc1F)c1c(F)ccc2c1NC(NCc1ncccc1F)=NS2(=O)=O] | 1.969 | 0.518 | 446.1 | 447.1 | [M + H]⁺ | 1 |
| 10-2A | 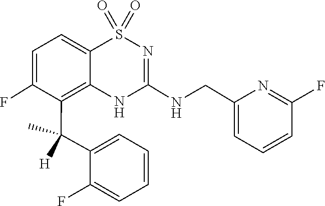 [SMILES: C[C@@H](c1ccccc1F)c1c(F)ccc2c1NC(NCc1cccc(F)n1)=NS2(=O)=O] | 1.607 | 0.519 | 446.1 | 447.1 | [M + H]⁺ | 1 |
| 10-2B | 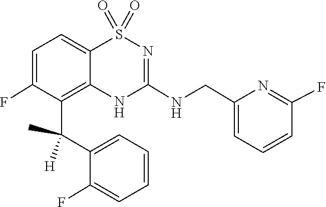 [SMILES: C[C@H](c1ccccc1F)c1c(F)ccc2c1NC(NCc1cccc(F)n1)=NS2(=O)=O] | 1.827 | 0.519 | 446.1 | 447.1 | [M + H]⁺ | 1 |
| 10-3A | 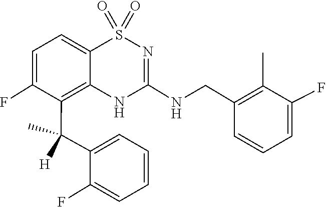 [SMILES: Cc1c(F)cccc1CNC1=NS(=O)(=O)c2ccc(F)c([C@@H](C)c3ccccc3F)c2N1] | 1.867 | 0.555 | 459.1 | 460.2 | [M + H]⁺ | 1 |
| 10-3B | 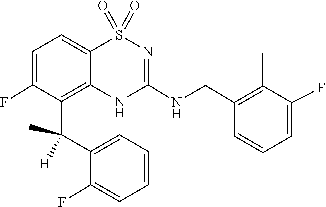 [SMILES: Cc1c(F)cccc1CNC1=NS(=O)(=O)c2ccc(F)c([C@H](C)c3ccccc3F)c2N1] | 2.148 | 0.554 | 459.1 | 460.2 | [M + H]⁺ | 1 |
| 10-4A | 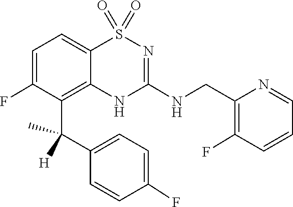 [SMILES: C[C@@H](c1ccc(F)cc1)c1c(F)ccc2c1NC(NCc1ncccc1F)=NS2(=O)=O] | 1.57 | 0.513 | 446.1 | 447.1 | [M + H]⁺ | 1 |
| 10-4B | 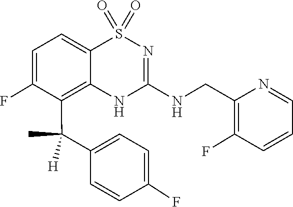 [SMILES: C[C@H](c1ccc(F)cc1)c1c(F)ccc2c1NC(NCc1ncccc1F)=NS2(=O)=O] | 1.638 | 0.509 | 446.1 | 447.1 | [M + H]⁺ | 1 |
| 10-5A | 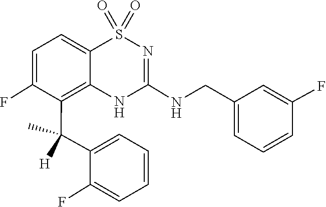 [SMILES: C[C@@H](c1ccccc1F)c1c(F)ccc2c1NC(NCc1cccc(F)c1)=NS2(=O)=O] | 1.593 | 0.539 | 445.1 | 446 | [M + H]⁺ | 1 |
| 10-5B | 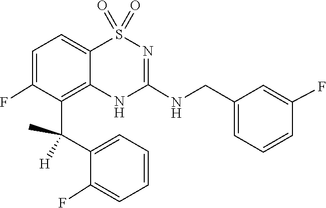 [SMILES: C[C@H](c1ccccc1F)c1c(F)ccc2c1NC(NCc1cccc(F)c1)=NS2(=O)=O] | 1.854 | 0.545 | 445.1 | 446 | [M + H]⁺ | 1 |
| 10-6A | 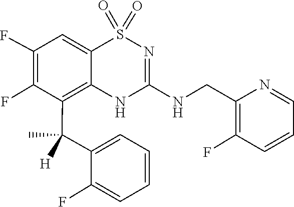 [SMILES: C[C@@H](c1ccccc1F)c1c(F)c(F)cc2c1NC(NCc1ncccc1F)=NS2(=O)=O] | 1.607 | 0.533 | 464.0 | 465 | [M + H]⁺ | 1 |
| 10-6B | 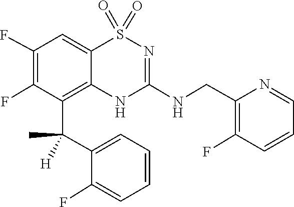 [SMILES: C[C@H](c1ccccc1F)c1c(F)c(F)cc2c1NC(NCc1ncccc1F)=NS2(=O)=O] | 1.827 | 0.533 | 464.0 | 465 | [M + H]⁺ | 1 |

### Example 11

### Synthesis of Representative Compound 11-1

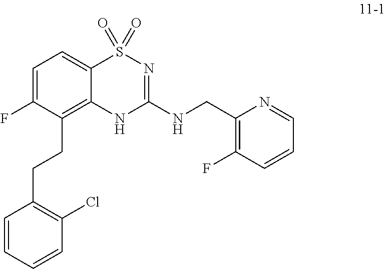 [SMILES: O=S1(=O)N=C(NCc2ncccc2F)Nc2c1ccc(F)c2CCc1ccccc1Cl]

### Step 11-1. Synthesis of (E)-2-(2-chlorostyryl)-3-fluoroaniline (INT 11A)

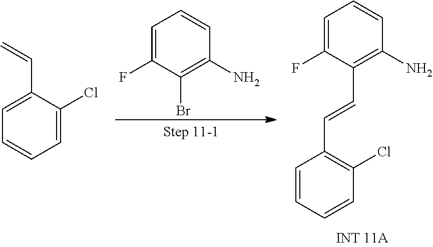

To a solution of 2-bromo-3-fluoro-aniline (2.5 g, 13.2 mmol, 1 eq) and 1-chloro-2-vinyl-benzene (2.74 g, 19.7 mmol, 1.5 eq) in DMF (40 mL) was added Pd(dppf)Cl₂ (481 mg, 656 mol, 0.05 eq) and TEA (2.91 g, 28.7 mmol, 4 mL, 2.2 eq). The reaction mixture was heated and stirred at 120° C. for 14 h. The reaction mixture was cooled to room temperature and poured into water (40 mL). The aqueous layer was extracted with EtOAc (2×30 mL). The combined organics were washed with brine (3×30 mL), dried over Na₂SO₄, filtered, and concentrated in vacuo to give a residue. The crude residue was purified by silica gel chromatography (0 to 15% EtOAc/petroleum ether) to afford (E)-2-(2-chlorostyryl)-3-fluoroaniline INT 11A (3.0 g, 9.7 mmol, 74% yield) that was used without further purification. LCMS (m/z) calculated for C₁₄H₁₁ClFN: 247.1; found 248.0 [M+H]⁺, t_{R}=0.618 min (Purity Method 1). ¹H NMR (400 MHz, DMSO-d6) δ=8.00 (dd, J=1.2, 7.8 Hz, 1H), 7.49-7.43 (m, 1H), 7.42-7.34 (m, 2H), 7.33-7.25 (m, 1H), 7.11 (d, J=16.4 Hz, 1H), 7.00-6.95 (m, 1H), 6.51 (d, J=8.1 Hz, 1H), 6.40-6.35 (m, 1H), 5.73 (s, 2H).

### Step 11-2. Synthesis of 2-(2-chlorophenethyl)-3-fluoroaniline (INT 11B)

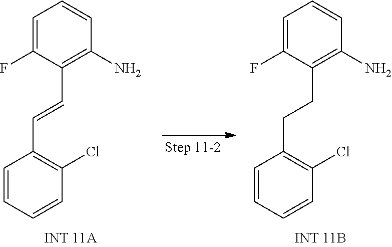 [SMILES: Nc1cccc(F)c1/C=C/c1ccccc1Cl ; Nc1cccc(F)c1CCc1ccccc1Cl]

To a solution of 2-[(E)-2-(2-chlorophenyl)vinyl]-3-fluoro-aniline INT 11A (3.00 g, 12.1 mmol, 1 eq) in MeOH (80 mL) was added Pd/C (0.2 g, 10% purity) under N₂ atmosphere. The suspension was degassed and purged with N₂ 3 times. The mixture was stirred under an atmosphere of H2 (15 Psi) at 25° C. for 2 h. The crude mixture was filtered through a pad of Celite and concentrated in vacuo. The crude residue was purified by silica gel chromatography (020% EtOAc/petroleum ether) to afford 2-(2-chlorophenethyl)-3-fluoroaniline INT 11B (2.1 g, 3.5 mmol, 29% yield) that was used without further purification. LCMS (m/z) calculated for C₁₄H₁₃ClFN: 249.1; found 250.1 [M+H]⁺, t_{R}=0.588 min (Purity Method 1).

### Step 11-3. Synthesis of 5-(2-chlorophenethyl)-6-fluoro-3-hydroxy-4H-benzo[e][1,2,4]thiadiazine 1,1-dioxide (INT 11C)

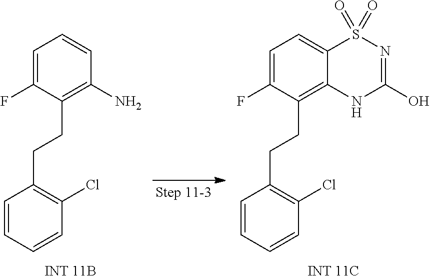 [SMILES: Nc1cccc(F)c1CCc1ccccc1Cl ; O=S1(=O)N=C(O)Nc2c1ccc(F)c2CCc1ccccc1Cl]

To a solution of N-(oxomethylene)sulfamoyl chloride (1.11 g, 7.81 mmol, 678 μL, 1.5 eq) in nitrobenzene (10 mL) at 0° C., was added a solution of 2-[2-(2-chlorophenyl)ethyl]-3-fluoro-aniline INT 11B (1.3 g, 5.21 mmol, 1 eq) in nitrobenzene (3 mL). The mixture was stirred at 0° C. for 0.5 h. Then AlCl₃ (1.04 g, 7.83 mmol, 428 μL, 1.5 eq) was added. The mixture was heated and stirred at 120° C. for 1.5 h. The reaction solution was cooled to room temperature and then diluted with saturated aqueous NH₄Cl (100 mL). The aqueous phase was extracted with EtOAc (3×100 mL). The combined organics were washed with brine (80 mL), dried over anhydrous sodium sulfate, filtered, and concentrated in vacuo to give a brown oil. The obtained brown oil was diluted with petroleum ether (300 mL) and then stirred for 30 min. The brown oil eventually formed a brown precipitate that was collected by vacuum filtration. The filter cake was washed with petroleum ether (200 mL) and then dried under high vacuum to afford 5-(2-chlorophenethyl)-6-fluoro-3-hydroxy-4H-benzo[e][1,2,4]thiadiazine 1,1-dioxide INT 11C (1.1 g, 2.3 mmol, 44% yield) that was used without further purification. LCMS (m/z) calculated for C₁₅H₁₂ClFN₂O₃S: 354.0; found 353.1 [M−H], t_{R}=0.420 min (Purity Method 2).

### Step 11-4. Synthesis of 3-chloro-5-(2-chlorophenethyl)-6-fluoro-4H-benzo[e][1,2,4]thiadiazine 1,1-dioxide (INT 11D)

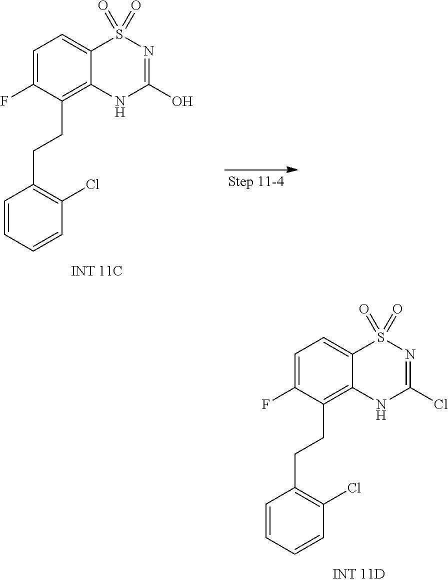 [SMILES: O=S1(=O)N=C(O)Nc2c1ccc(F)c2CCc1ccccc1Cl ; O=S1(=O)N=C(Cl)Nc2c1ccc(F)c2CCc1ccccc1Cl]

To a solution of 5-[2-(2-chlorophenyl)ethyl]-6-fluoro-1,1-dioxo-4H-1,2,4-benzothiadiazin-3-ol INT 11C (0.5 g, 1.4 mmol, 1 eq) in POCl₃ (5 mL), was added N,N-diethylaniline (373 mg, 2.50 mmol, 0.4 mL, 1.77 eq). The reaction mixture was heated and stirred at 120° C. for 2 h. The reaction mixture was cooled to room temperature and poured into water (50 mL) portionwise while the temperature was kept below 35° C. The obtained mixture was stirred for 1 h. The aqueous layer was extracted with EtOAc (3×80 mL). The combined organics were washed with brine (2×60 mL), dried over Na₂SO₄, filtered, and concentrated in vacuo to give a residue. The crude residue was purified by silica gel chromatography (0 to 50% EtOAc/petroleum ether) to afford 3-chloro-5-(2-chlorophenethyl)-6-fluoro-4H-benzo[e][1,2,4]thiadiazine 1,1-dioxide INT 11D (80 mg, 197 mol, 14% yield). LCMS (m/z) calculated for C₁₅H₁₁ClFN₂O₂S: 372.0; found 371.0 [M−H], t_{R}=0.520 min (Purity Method 2).

### Step 11-5. Synthesis of 5-(2-chlorophenethyl)-6-fluoro-3-(((3-fluoropyridin-2-yl)methyl)amino)-4H-benzo[e][1,2,4]thiadiazine 1,1-dioxide (Compound 11-1)

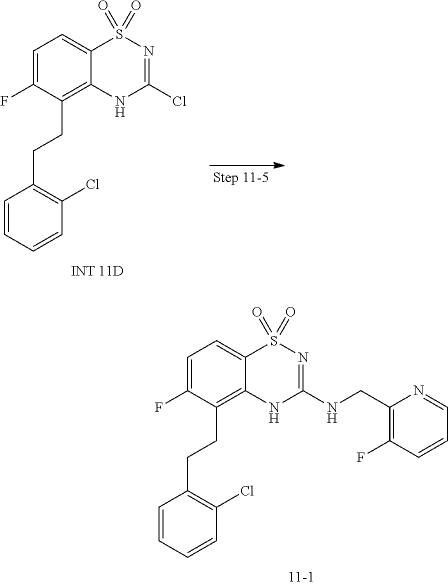 [SMILES: O=S1(=O)N=C(Cl)Nc2c1ccc(F)c2CCc1ccccc1Cl ; O=S1(=O)N=C(NCc2ncccc2F)Nc2c1ccc(F)c2CCc1ccccc1Cl]

To a solution of (3-fluoro-2-pyridyl)methanamine dihydrochloride (64.0 mg, 322 mol, 1.5 eq) and 3-chloro-5-[2-(2-chlorophenyl)ethyl]-6-fluoro-4H-1,2,4-benzothiadiazine 1,1-dioxide INT 11D (80 mg, 214 mol, 1 eq) in DMAC (2 mL), was added DIPEA (166 mg, 1.29 mmol, 224 μL, 6 eq). The reaction mixture was heated and stirred at 120° C. for 1 h. The reaction mixture was cooled to room temperature and poured into water (40 mL). The aqueous layer was extracted with EtOAc (2×30 mL). The combined organics were washed with brine (3×30 mL), dried over Na₂SO₄, filtered, and concentrated in vacuo to give a residue. The residue was purified by preparative HPLC (column: Phenomenex luna C18 150×25 mm×10 um; A-B mobile phase: [water(FA)-ACN]; gradient:40%-70% B over 10 min) and lyophilized to afford 5-(2-chlorophenethyl)-6-fluoro-3-(((3-fluoropyridin-2-yl)methyl)amino)-4H-benzo[e][1,2,4]thiadiazine 1,1-dioxide 11-1 (39.2 mg, 84.7 mol, 40% yield). LCMS (m/z) calculated for C₂₁H₁₇ClF₂N₄O₂S: 462.1; found 463.1 [M+H]⁺, t_{R}=0.528 min (Purity Method 1). H NMR (400 MHz, DMSO-d6) δ=10.40-9.80 (m, 1H), 8.48 (br d, J=4.5 Hz, 1H), 8.22-8.03 (m, 1H), 7.80 (br t, J=9.1 Hz, 1H), 7.61 (br dd, J=6.3, 7.8 Hz, 1H), 7.50 (td, J=4.4, 8.4 Hz, 1H), 7.43-7.34 (m, 1H), 7.32-7.18 (m, 3H), 7.00 (br t, J=8.8 Hz, 1H), 4.70 (br d, J=3.6 Hz, 2H), 3.17-3.02 (m, 2H), 3.01-2.90 (m, 2H).

### Synthesis of Scheme 11 Compounds

The compounds listed in Table 12 were made using the procedures of Example 11.

TABLE 12

| | | Purity | Exact | | | |
|---|---|---|---|---|---|---|
| Cpd | | RT | mass | observed | | Purity |
| No. | Structure | (min) | (g/mol) | m/z | Ion | Method |
| 11-1 | 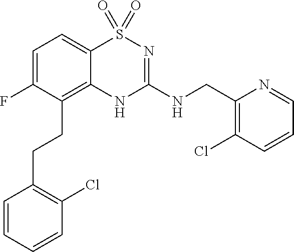 [SMILES: O=S1(=O)N=C(NCc2ncccc2Cl)Nc2c1ccc(F)c2CCc1ccccc1Cl] | 0.519 | 462.1 | 463.1 | [M + H]⁺ | 1 |

### Example 12

### Synthesis of Representative Compound 12-1

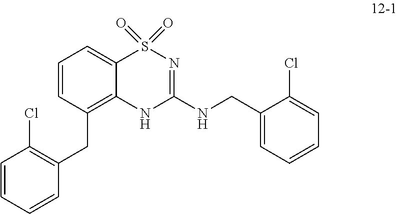 [SMILES: O=S1(=O)N=C(NCc2ccccc2Cl)Nc2c(Cc3ccccc3Cl)cccc21]

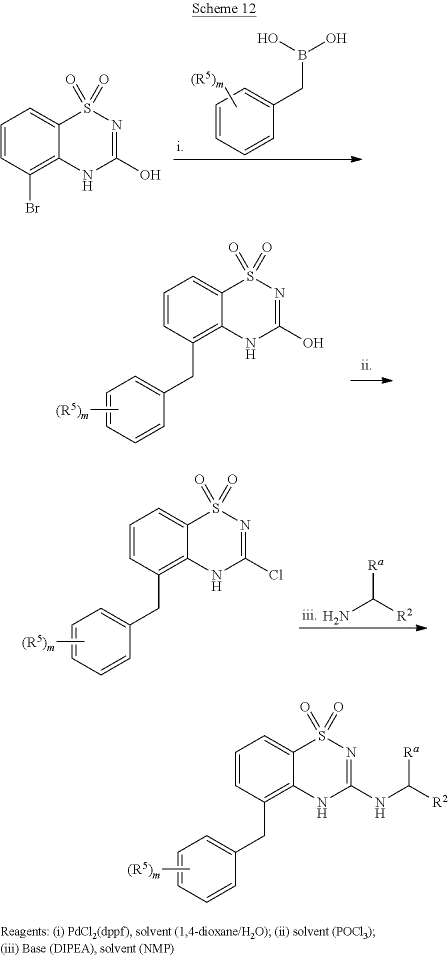

### Step 12-1. Synthesis of 5-(2-chlorobenzyl)-3-hydroxy-4H-benzo[e][1,2,4]thiadiazine 1,1-dioxide (INT 12A)

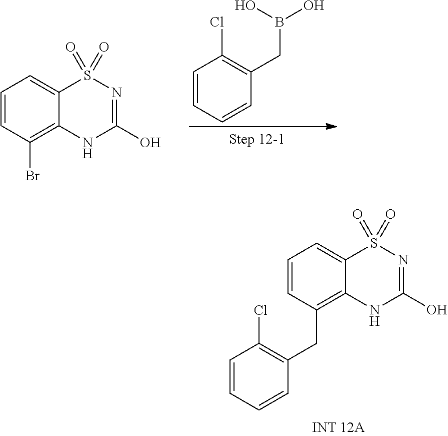 [SMILES: O=S1(=O)N=C(O)Nc2c(Br)cccc21 ; OB(O)Cc1ccccc1Cl ; O=S1(=O)N=C(O)Nc2c(Cc3ccccc3Cl)cccc21]

To a solution of 5-bromo-3-hydroxy-4H-benzo[e][1,2,4]thiadiazine 1,1-dioxide (100 mg, 1 Eq, 361 μmol) and (2-chlorobenzyl)boronic acid (123 mg, 2 Eq, 722 μmol) in 1,4-Dioxane (4 mL) and water (4 mL), PdCl₂(dppf) (26.4 mg, 0.1 Eq, 36.1 μmol), Cs₂CO₃ (353 mg, 3 Eq, 1.08 mmol) were added. The mixture was degasssed with N₂ and then the reaction was heated and stirred at 90° C. for 3 h. The reaction mixture was cooled and passed through a syringe filter into a test tube, and the filtrate was directly loaded/purified by HPLC to yield 5-(2-chlorobenzyl)-3-hydroxy-4H-benzo[e][1,2,4]thiadiazine 1,1-dioxide INT 12A (83 mg, 0.26 mmol, 71% yield). LCMS (m/z) calculated for C₁₄H₁₁ClN₂O₃S: 322.0; found 323.0 [M+H]⁺, t_{R}=4.575 min (Purity Method 3). ¹H NMR (400 MHz, DMSO) δ 10.55-10.31 (m, 1H), 7.69 (dd, J=7.9, 1.4 Hz, 1H), 7.56-7.45 (m, 1H), 7.40-7.28 (m, 2H), 7.21 (t, J=7.7 Hz, 1H), 7.16-7.09 (m, 1H), 7.05 (dd, J=7.8, 1.5 Hz, 1H), 4.23 (s, 2H).

### Step 12-2. Synthesis of 3-chloro-5-(2-chlorobenzyl)-4H-benzo[e][1,2,4]thiadiazine 1,1-dioxide (INT 12B)

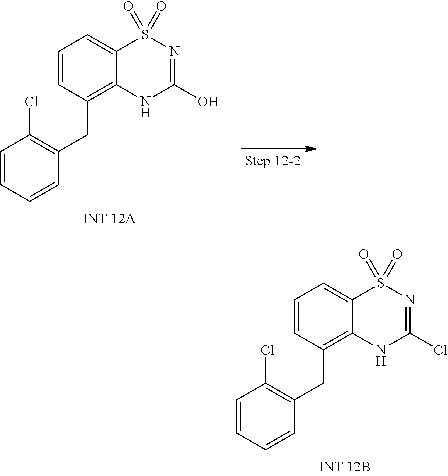 [SMILES: O=S1(=O)N=C(O)Nc2c(Cc3ccccc3Cl)cccc21 ; O=S1(=O)N=C(Cl)Nc2c(Cc3ccccc3Cl)cccc21]

To a stirring solution of 5-(2-chlorobenzyl)-3-hydroxy-4H-benzo[e][1,2,4]thiadiazine 1,1-dioxide INT 12A (83 mg, 1 Eq, 0.26 mmol) was added phosphorus oxychloride (0.39 g, 0.24 mL, 10 Eq, 2.6 mmol). The mixture was heated to 125° C. and stirred for 18 h. The reaction mixture was cooled to room temperature and concentrated in vacuo to afford 3-chloro-5-(2-chlorobenzyl)-4H-benzo[e][1,2,4]thiadiazine 1,1-dioxide INT 12B (24 mg, 67 μmol, 26% yield) that was used without further purification. LCMS (m/z) calculated for C₁₄H₁₀Cl₂N₂O₂S: 340.0; found 341.0 [M+H]⁺, t_{R}=6.016 min (Purity Method 3). ¹H NMR (400 MHz, DMSO) δ 7.77 (dd, J=7.9, 1.5 Hz, 1H), 7.57-7.48 (m, 1H), 7.41 (t, J=7.8 Hz, 1H), 7.38-7.28 (m, 2H), 7.14 (ddd, J=9.2, 6.9, 3.1 Hz, 2H), 4.33 (s, 2H).

### Step 12-3. Synthesis of 5-(2-chlorobenzyl)-3-((2-chlorobenzyl)amino)-4H-benzo[e][1,2,4]thiadiazine 1,1-dioxide (Compound 12-1)

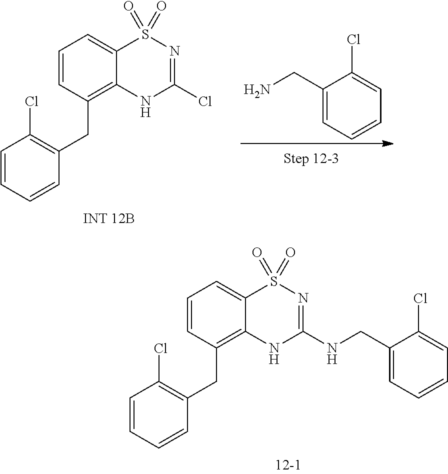 [SMILES: O=S1(=O)N=C(Cl)Nc2c(Cc3ccccc3Cl)cccc21 ; NCc1ccccc1Cl ; O=S1(=O)N=C(NCc2ccccc2Cl)Nc2c(Cc3ccccc3Cl)cccc21]

To a stirring solution of 3-chloro-5-(2-chlorobenzyl)-4H-benzo[e][1,2,4]thiadiazine 1,1-dioxide INT 12B (10 mg, 1 Eq, 29 μmol) in NMP (2 mL) was added (2-chlorophenyl)methanamine (6.2 mg, 1.5 Eq, 44 μmol). The mixture was heated to 100° C. and stirred for 18 h. The reaction mixture was cooled to room temperature and purified by reversed phase HPLC to afford 5-(2-chlorobenzyl)-3-((2-chlorobenzyl)amino)-4H-benzo[e][1,2,4]thiadiazine 1,1-dioxide 12-1 (5.2 mg, 12 μmol, 40% yield). LCMS (m/z) calculated for C₂₁H₁₇Cl₂N₃O₂S: 445.0; found 446.0 [M+H]⁺, t_{R}=9.284 min (Purity Method 3).

### Synthesis of Scheme 12 Compounds

Alternatively for Step 12-3, the following compounds were made using 3 eq of DIPEA in NMP at 120° C. for 18 h: 12-3.

Alternatively for Step 12-3, the following compounds were made using 3 eq of DIPEA in NMP at 130° C. for 18 h: 12-4, 12-5, 12-6, 12-7, 12-8, 12-9, 12-10, 12-11, 12-12, 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 12-3 1, 12-32.

The compounds listed in Table 13 were made using the procedures of Scheme 12.

TABLE 13

| | | Purity | Exact | | | |
|---|---|---|---|---|---|---|
| Cpd | | RT | mass | observed | | Purity |
| No. | Structure | (min) | (g/mol) | m/z | Ion | Method |
| 12-1 | 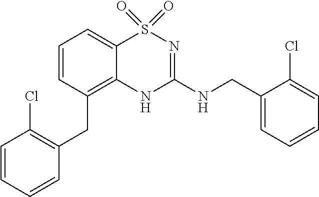 [SMILES: O=S1(=O)N=C(NCc2ccccc2Cl)Nc2c(Cc3ccccc3Cl)cccc21] | 9.284 | 445.0 | 446.2 | [M + H]⁺ | 3 |
| 12-2 | 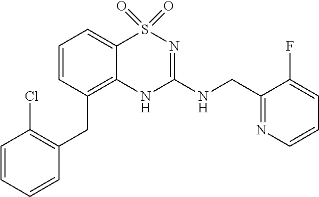 [SMILES: O=S1(=O)N=C(NCc2ncccc2F)Nc2c(Cc3ccccc3Cl)cccc21] | 8.129 | 430.0 | 431.2 | [M + H]⁺ | 3 |
| 12-3 | 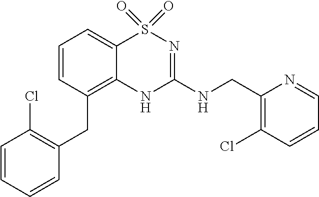 [SMILES: O=S1(=O)N=C(NCc2ncccc2Cl)Nc2c(Cc3ccccc3Cl)cccc21] | 8.701 | 446.0 | 447.2 | [M + H]⁺ | 3 |
| 12-4 | 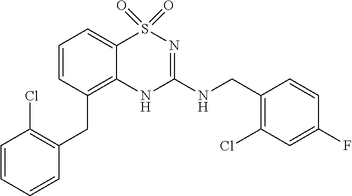 [SMILES: O=S1(=O)N=C(NCc2ccc(F)cc2Cl)Nc2c(Cc3ccccc3Cl)cccc21] | 9.369 | 463.0 | 464.0 | [M + H]⁺ | 3 |
| 12-5 | 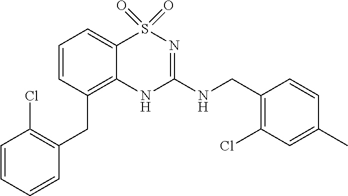 [SMILES: Cc1ccc(CNC2=NS(=O)(=O)c3cccc(Cc4ccccc4Cl)c3N2)c(Cl)c1] | 9.724 | 459.0 | 460.0 | [M + H]⁺ | 3 |
| 12-6 | 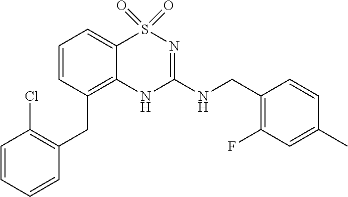 [SMILES: Cc1ccc(CNC2=NS(=O)(=O)c3cccc(Cc4ccccc4Cl)c3N2)c(F)c1] | 9.502 | 443.1 | 444.0 | [M + H]⁺ | 3 |
| 12-7 | 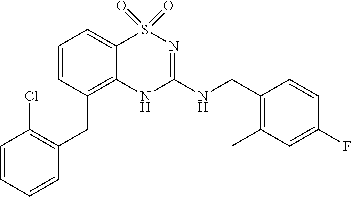 [SMILES: Cc1cc(F)ccc1CNC1=NS(=O)(=O)c2cccc(Cc3ccccc3Cl)c2N1] | 9.376 | 443.1 | 444.0 | [M + H]⁺ | 3 |
| 12-8 | 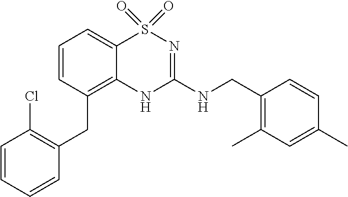 [SMILES: Cc1ccc(CNC2=NS(=O)(=O)c3cccc(Cc4ccccc4Cl)c3N2)c(C)c1] | 9.697 | 439.1 | 440.0 | [M + H]⁺ | 3 |
| 12-9 | 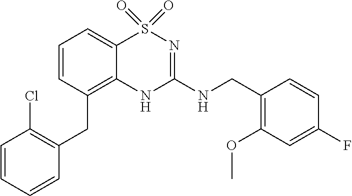 [SMILES: COc1cc(F)ccc1CNC1=NS(=O)(=O)c2cccc(Cc3ccccc3Cl)c2N1] | 9.286 | 459.1 | 460.0 | [M + H]⁺ | 3 |
| 12-10 | 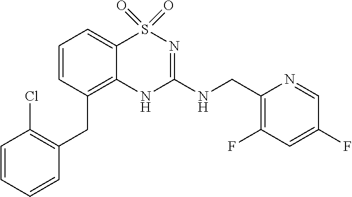 [SMILES: O=S1(=O)N=C(NCc2ncc(F)cc2F)Nc2c(Cc3ccccc3Cl)cccc21] | 8.508 | 448.1 | 449.0 | [M + H]⁺ | 3 |
| 12-11 | 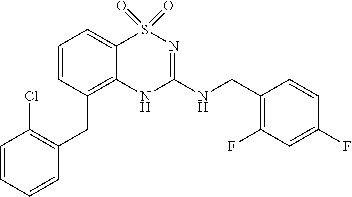 [SMILES: O=S1(=O)N=C(NCc2ccc(F)cc2F)Nc2c(Cc3ccccc3Cl)cccc21] | 9.083 | 447.1 | 448.0 | [M + H]⁺ | 3 |
| 12-12 | 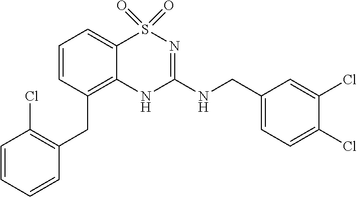 [SMILES: O=S1(=O)N=C(NCc2ccc(Cl)c(Cl)c2)Nc2c(Cc3ccccc3Cl)cccc21] | 9.814 | 479.0 | 480.0 | [M + H]⁺ | 3 |
| 12-13 | 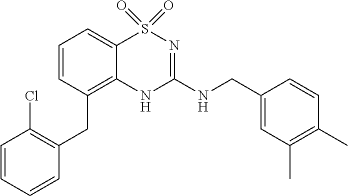 [SMILES: Cc1ccc(CNC2=NS(=O)(=O)c3cccc(Cc4ccccc4Cl)c3N2)cc1C] | 9.609 | 439.1 | 440.0 | [M + H]⁺ | 3 |
| 12-14 | 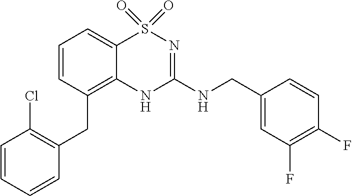 [SMILES: O=S1(=O)N=C(NCc2ccc(F)c(F)c2)Nc2c(Cc3ccccc3Cl)cccc21] | 9.076 | 447.0 | 448.0 | [M + H]⁺ | 3 |
| 12-15 | 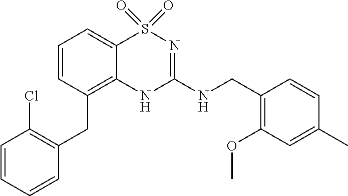 [SMILES: COc1cc(C)ccc1CNC1=NS(=O)(=O)c2cccc(Cc3ccccc3Cl)c2N1] | 9.499 | 455.1 | 456.0 | [M + H]⁺ | 3 |
| 12-16 | 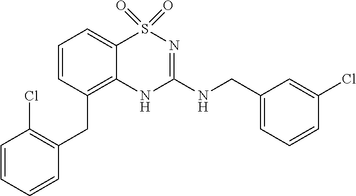 [SMILES: O=S1(=O)N=C(NCc2cccc(Cl)c2)Nc2c(Cc3ccccc3Cl)cccc21] | 9.273 | 445.0 | 446.0 | [M + H]⁺ | 3 |
| 12-17 | 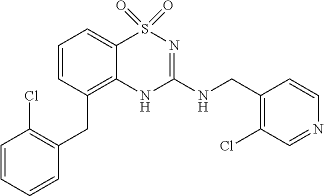 [SMILES: O=S1(=O)N=C(NCc2ccncc2Cl)Nc2c(Cc3ccccc3Cl)cccc21] | 7.699 | 446.0 | 447.0 | [M + H]⁺ | 3 |
| 12-18 | 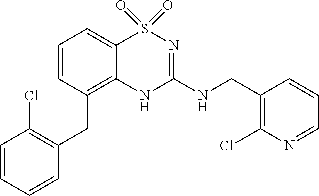 [SMILES: O=S1(=O)N=C(NCc2cccnc2Cl)Nc2c(Cc3ccccc3Cl)cccc21] | 7.943 | 446.0 | 447.0 | [M + H]⁺ | 3 |
| 12-19 | 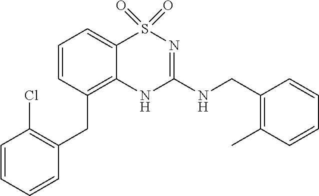 [SMILES: Cc1ccccc1CNC1=NS(=O)(=O)c2cccc(Cc3ccccc3Cl)c2N1] | 9.428 | 425.1 | 426.0 | [M + H]⁺ | 3 |
| 12-20 | 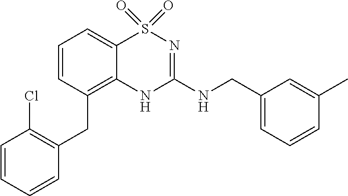 [SMILES: Cc1cccc(CNC2=NS(=O)(=O)c3cccc(Cc4ccccc4Cl)c3N2)c1] | 9.249 | 425.1 | 426.0 | [M + H]⁺ | 3 |
| 12-21 | 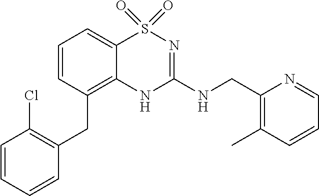 [SMILES: Cc1cccnc1CNC1=NS(=O)(=O)c2cccc(Cc3ccccc3Cl)c2N1] | 6.316 | 426.1 | 427.0 | [M + H]⁺ | 3 |
| 12-22 | 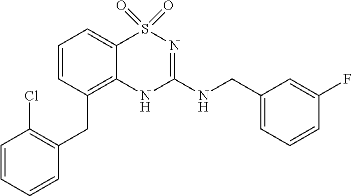 [SMILES: O=S1(=O)N=C(NCc2cccc(F)c2)Nc2c(Cc3ccccc3Cl)cccc21] | 9.024 | 429.1 | 430.0 | [M + H]⁺ | 3 |
| 12-23 | 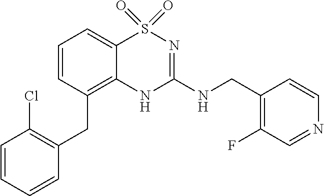 [SMILES: O=S1(=O)N=C(NCc2ccncc2F)Nc2c(Cc3ccccc3Cl)cccc21] | 7.32 | 430.1 | 431.0 | [M + H]⁺ | 3 |
| 12-24 | 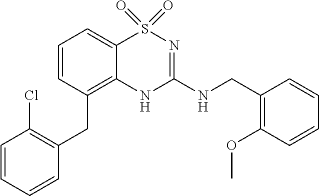 [SMILES: COc1ccccc1CNC1=NS(=O)(=O)c2cccc(Cc3ccccc3Cl)c2N1] | 9.048 | 441.1 | 442.0 | [M + H]⁺ | 3 |
| 12-25 | 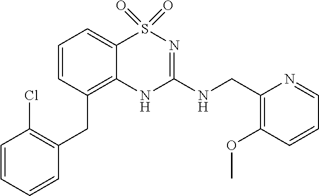 [SMILES: COc1cccnc1CNC1=NS(=O)(=O)c2cccc(Cc3ccccc3Cl)c2N1] | 7.485 | 442.1 | 443.0 | [M + H]⁺ | 3 |
| 12-26 | 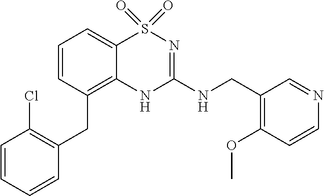 [SMILES: COc1ccncc1CNC1=NS(=O)(=O)c2cccc(Cc3ccccc3Cl)c2N1] | 6.036 | 442.1 | 443.0 | [M + H]⁺ | 3 |
| 12-27 | 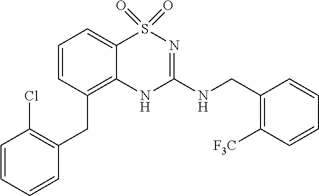 [SMILES: O=S1(=O)N=C(NCc2ccccc2C(F)(F)F)Nc2c(Cc3ccccc3Cl)cccc21] | 8.513 | 479.1 | 481.0 | [M + H]⁺ | 3 |
| 12-28 | 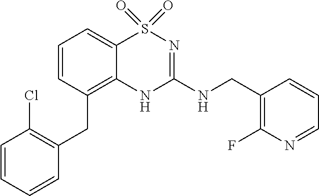 [SMILES: O=S1(=O)N=C(NCc2cccnc2F)Nc2c(Cc3ccccc3Cl)cccc21] | 7.832 | 430.1 | 431.0 | [M + H]⁺ | 3 |
| 12-29 | 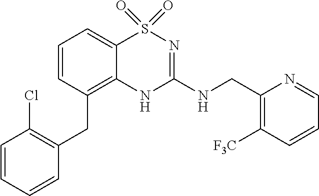 [SMILES: O=S1(=O)N=C(NCc2ncccc2C(F)(F)F)Nc2c(Cc3ccccc3Cl)cccc21] | 9.913 | 480.1 | 481.0 | [M + H]⁺ | 3 |
| 12-30 | 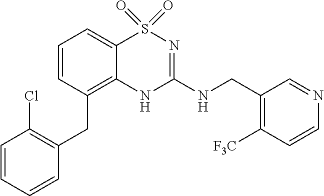 [SMILES: O=S1(=O)N=C(NCc2cnccc2C(F)(F)F)Nc2c(Cc3ccccc3Cl)cccc21] | 8.245 | 480.1 | 481.0 | [M + H]⁺ | 3 |
| 12-31 | 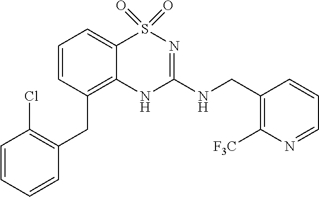 [SMILES: O=S1(=O)N=C(NCc2cccnc2C(F)(F)F)Nc2c(Cc3ccccc3Cl)cccc21] | 8.513 | 480.1 | 481.0 | [M + H]⁺ | 3 |
| 12-32 | 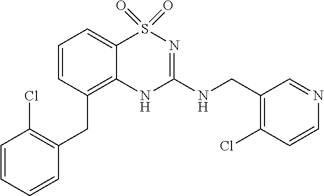 [SMILES: O=S1(=O)N=C(NCc2cnccc2Cl)Nc2c(Cc3ccccc3Cl)cccc21] | 7.226 | 446.0 | 447.0 | [M + H]⁺ | 3 |

### Example 13

### Synthesis of Representative Compound 13-1

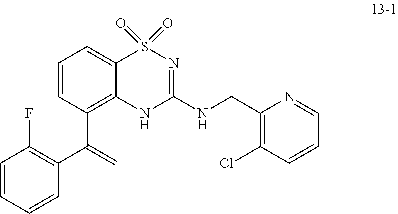 [SMILES: C=C(c1ccccc1F)c1cccc2c1NC(NCc1ncccc1Cl)=NS2(=O)=O]

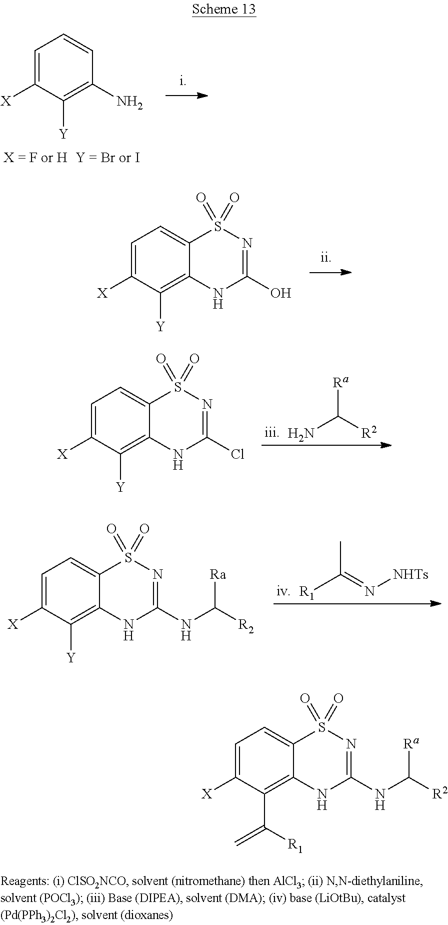

### Step 13-1. Synthesis of 5-bromo-3-hydroxy-4H-benzo[e][1,2,4]thiadiazine 1,1-dioxide (INT 13A)

To a solution of N-(oxomethylene)sulfamoyl chloride (5.81 g, 41.1 mmol, 3.57 mL, 1.41 eq) in nitrobenzene (80 mL) was added 2-bromoaniline (5 g, 29.1 mmol, 3.16 mL, 1 eq) at 0° C. The mixture was stirred at 0° C. for 0.5 h. Then, AlCl₃ (5.81 g, 43.6 mmol, 2.38 mL, 1.5 eq) was added. The mixture was stirred at 120° C. for 1.5 h. The reaction solution was cooled to room temperature and then diluted with saturated NH₄Cl aqueous solution (500 mL). The aqueous phase was extracted with ethyl acetate (3×200 mL). The combined organic layers were washed with brine (200 mL), dried over anhydrous sodium sulfate, filtered, and concentrated under vacuum to give a brown oil. The obtained brown oil was diluted with petroleum ether (300 mL) and then stirred for 30 min. The brown oil eventually formed a brown precipitate that was collected by vacuum filtration. The filter cake was washed with petroleum ether (200 mL) and then dried under vacuum to give 5-bromo-3-hydroxy-4H-benzo[e][1,2,4]thiadiazine 1,1-dioxide INT 13A (4.0 g, 14.2 mmol, 49% yield). ¹H NMR (400 MHz, DMSO-d6) δ=10.25 (s, 1H), 7.96 (dd, J=1.1, 7.9 Hz, 1H), 7.82 (d, J=7.7 Hz, 1H), 7.25 (t, J=7.9 Hz, 1H).

### Step 13-2. Synthesis of 5-bromo-3-chloro-4H-benzo[e][1,2,4]thiadiazine 1,1-dioxide (INT 13B)

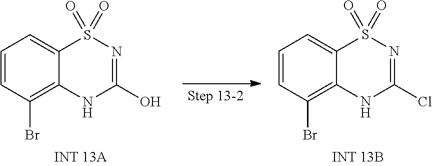 [SMILES: O=S1(=O)N=C(O)Nc2c(Br)cccc21 ; O=S1(=O)N=C(Cl)Nc2c(Br)cccc21]

To a solution of 5-bromo-3-hydroxy-4H-benzo[e][1,2,4]thiadiazine 1,1-dioxide INT 13A (2.2 g, 7.94 mmol, 1 eq) in POCl₃ (10 mL) was added N,N-diethylaniline (1.78 g, 11.9 mmol, 1.90 mL, 1.5 eq). The mixture was stirred at 120° C. for 2 h. The reaction solution was then poured into water (500 mL) portionwise while the temperature was kept below 35° C. The obtained mixture was stirred for 1 h. The dark brown oil eventually formed a brown precipitate that was collected by vacuum filtration and dried under vacuum to give 5-bromo-3-chloro-4H-benzo[e][1,2,4]thiadiazine 1,1-dioxide INT 13B (2.0 g, 6.4 mmol, 80% yield) that was used without further purification. The filtrate was adjusted to pH=7 by KOH and then discarded. ¹H NMR (400 MHz, DMSO-d6) δ=7.93 (d, J=7.8 Hz, 1H), 7.79 (d, J=7.8 Hz, 1H), 7.30 (t, J=7.8 Hz, 1H).

### Step 13-3. Synthesis of 5-bromo-3-(((3-chloropyridin-2-yl)methyl)amino)-4H-benzo[e][1,2,4]thiadiazine 1,1-dioxide (INT 13C)

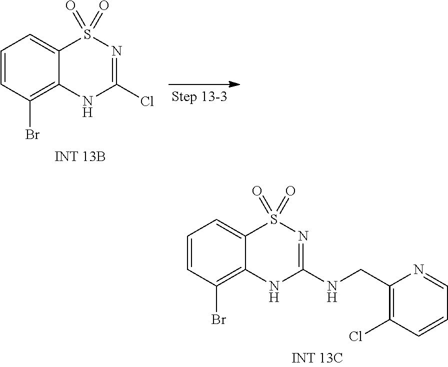 [SMILES: O=S1(=O)N=C(Cl)Nc2c(Br)cccc21 ; O=S1(=O)N=C(NCc2ncccc2Cl)Nc2c(Br)cccc21]

To a solution of (3-chloro-2-pyridyl)methanamine (1.12 g, 5.21 mmol, 1.1 eq) and 5-bromo-3-chloro-4H-benzo[e][1,2,4]thiadiazine 1,1-dioxide INT 13B (1.4 g, 4.74 mmol, 1 eq) in DMA (10 mL) was added DIPEA (1.84 g, 14.21 mmol, 2.48 mL, 3 eq). The reaction mixture was stirred at 120° C. for 4 h. The reaction mixture was poured into water (40 mL). The product was extracted with EtOAc (2×30 mL). The combined organics were washed with brine (3×30 mL), dried over Na₂SO₄, filtered, and concentrated in vacuo to give a crude residue. The residue was purified by flash silica gel chromatography (0á90% EtOAc/petroleum ether) to afford 5-bromo-3-(((3-chloropyridin-2-yl)methyl)amino)-4H-benzo[e][1,2,4]thiadiazine 1,1-dioxide INT 13C (500 mg, 1.11 mmol, 23% yield). LCMS (m/z) calculated for C₁₃H₁₀BrClN₄O₂S: 399.9; found 401.0 [M+H]⁺, t_{R}=0.453 min (Purity Method 1). ¹H NMR (400 MHz, DMSO-d6) δ=10.24 (br s, 1H), 8.72 (br s, 1H), 8.59 (dd, J=1.2, 4.7 Hz, 1H), 8.02 (dd, J=1.3, 8.0 Hz, 1H), 7.91 (d, J=8.0 Hz, 1H), 7.73 (d, J=7.9 Hz, 1H), 7.46 (dd, J=4.6, 8.1 Hz, 1H), 7.23 (t, J=7.8 Hz, 1H), 4.72 (d, J=4.9 Hz, 2H).

### Step 13-4. Synthesis of 3-(((3-chloropyridin-2-yl)methyl)amino)-5-(1-(2-fluorophenyl)vinyl)-4H-benzo[e][1,2,4]thiadiazine 1,1-dioxide (13-1)

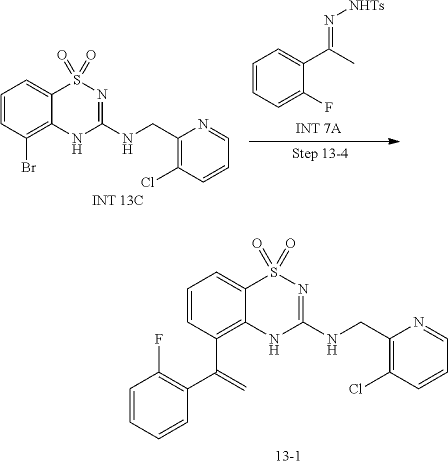

To a solution of N-[(E)-1-(2-fluorophenyl)ethylideneamino]-4-methyl-benzenesulfonamide INT 13D (199 mg, 648 mol, 1.45 eq) and 5-bromo-N-[(3-chloro-2-pyridyl)methyl]-1,1-dioxo-4H-1,2,4-benzothiadiazin-3-amine INT 7A (180 mg, 448 mol, 1 eq) in dioxane (2 mL) were added lithium tert-butoxide (108 mg, 1.34 mmol, 121 μL, 3 eq) and Pd(PPh₃)₂Cl₂ (31.4 mg, 44.8 mol, 0.1 eq). The reaction mixture was stirred at 100° C. for 15 h. The reaction mixture was cooled to room temperature and extracted with ethyl acetate (20 mL). The organic phase was washed with brine (20 mL), dried over anhydrous sodium sulfate, filtered and concentrated under reduced pressure to give a residue. The residue was purified by prep-HPLC (column: Waters Xbridge Prep OBD C18 150×40 mm×10 um; A-B mobile phase: [water(NH₄HCO₃)-ACN]; B: 28%-58% over 10 min) to afford 3-(((3-chloropyridin-2-yl)methyl)amino)-5-(1-(2-fluorophenyl)vinyl)-4H-benzo[e][1,2,4]thiadiazine 1,1-dioxide 13-1 (18.8 mg, 40.4 mol, 9% yield). LCMS (m/z) calculated for C₂₁H₁₆ClFN₄O₂S: 442.1; found 443.2 [M+H]⁺, t_{R}=0.502 min (Purity Method 1). ¹H NMR (400 MHz, DMSO-d6) δ=10.08 (s, 1H), 8.54 (d, JJ=4.4 Hz, 1H), 8.38 (br t, J=4.4 Hz, 1H), 8.00 (dd, J=1.1, 8.0 Hz, 1H), 7.69 (dd, J=3.1, 6.1 Hz, 1H), 7.49-7.36 (m, 2H), 7.34-7.15 (m, 5H), 6.14 (s, 1H), 5.67 (s, 1H), 4.69 (br d, J=4.1 Hz, 2H), 2.07 (s, 1H).

### Synthesis of Scheme 13 Compounds

Alternatively for Step 13-1, the following compound was made using 2-iodoaniline instead of 2-bromoaniline: 13-2.

Alternatively for Step 13-1, the following compound was made using 2-bromo-3-fluoroaniline instead of 2-bromoaniline: 13-3.

Alternatively for Step 13-4, the following compounds were made using 0.13 eq of Xphos, 0.11 eq of Pd₂(dba)₃, and 1.9 eq of t-BuOLi at 100° C. for 15 h: 13-2.

Alternatively for Step 13-4 to make 13-3, 1.6 eq of synthesized 2-[1-(2-fluorophenyl)vinyl]-4,4,5,5-tetramethyl-1,3,2-dioxaborolane was reacted with 1 eq of the synthesized 5-bromo-6-fluoro-N-[(3-fluoro-2-pyridyl)methyl]-1,1-dioxo-4H-1,2,4-benzothiadiazin-3-amine, 2 eq of K₂CO₃, and 0.13 eq of Pd(dppf)C12 in 5:1 dioxane/H₂O at 80° C. for 15 h.

The compounds listed in Table 14 were made using the procedures of Scheme 13.

TABLE 14

| | | Purity | Exact | | | |
|---|---|---|---|---|---|---|
| Cpd | | RT | mass | observed | | Purity |
| No. | Structure | (min) | (g/mol) | m/z | Ion | Method |
| 13-1 | 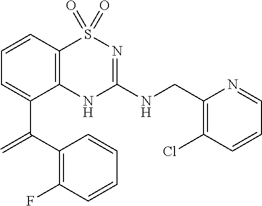 [SMILES: C=C(c1ccccc1F)c1cccc2c1NC(NCc1ncccc1Cl)=NS2(=O)=O] | 0.52 | 442.1 | 443.2 | [M + H]⁺ | 1 |
| 13-2 | 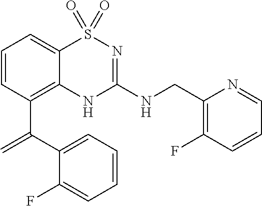 [SMILES: C=C(c1ccccc1F)c1cccc2c1NC(NCc1ncccc1F)=NS2(=O)=O] | 0.51 | 426.1 | 426.9 | [M + H]⁺ | 1 |
| 13-3 | 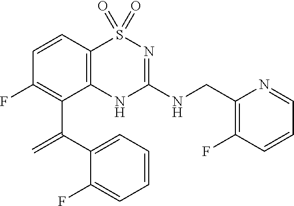 [SMILES: C=C(c1ccccc1F)c1c(F)ccc2c1NC(NCc1ncccc1F)=NS2(=O)=O] | 0.505 | 444.1 | 445.2 | [M + H]⁺ | 1 |

### Example 14

### Synthesis of Representative Compound 14-1

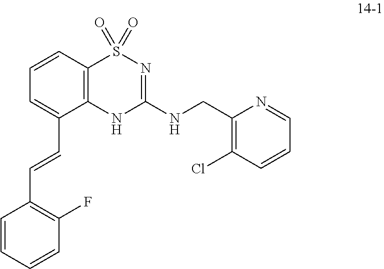 [SMILES: O=S1(=O)N=C(NCc2ncccc2Cl)Nc2c(/C=C/c3ccccc3F)cccc21]

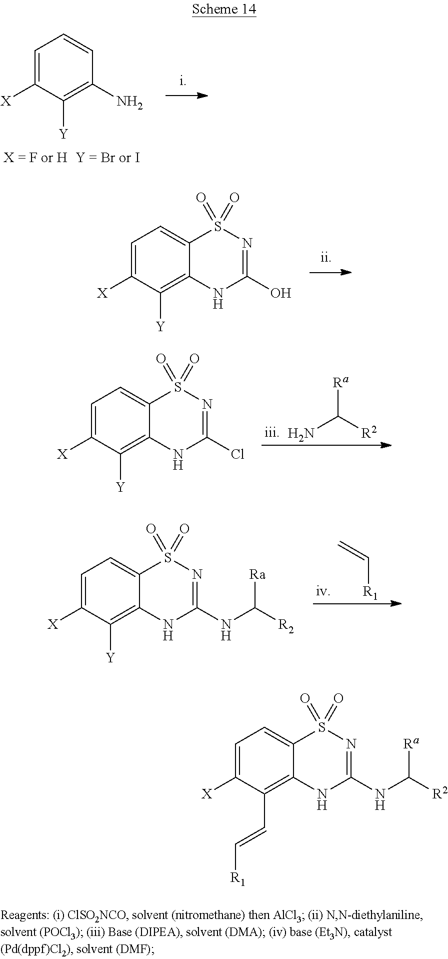

### Step 14-1. Synthesis of 3-hydroxy-5-iodo-4H-benzo[e][1,2,4]thiadiazine 1,1-dioxide (INT 14A)

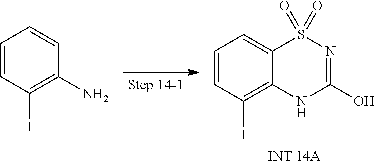 [SMILES: Nc1ccccc1I ; O=S1(=O)N=C(O)Nc2c(I)cccc21]

To a solution of N-(oxomethylene)sulfamoyl chloride (4.52 g, 32.0 mmol, 2.78 mL, 1.4 eq) in nitrobenzene (50 mL) was added 2-iodoaniline (5.0 g, 22.8 mmol, 1 eq) at 0° C. The mixture was stirred at 0° C. for 0.5 h. Then, AlCl₃ (4.57 g, 34.2 mmol, 1.87 mL, 1.5 eq) was added. The mixture was stirred at 120° C. for 1.5 h. The reaction solution was cooled to room temperature and then diluted with saturated NH₄Cl aqueous solution (500 mL); the aqueous layer was extracted with ethyl acetate (3×200 mL). The combined organic layers were washed with brine (200 mL), dried over anhydrous sodium sulfate, filtered, and concentrated in vacuo to give a brown oil. The obtained brown oil was diluted with petroleum ether (300 mL) and then stirred for 30 min. The brown oil eventually formed a brown precipitate that was collected by vacuum filtration. The filter cake was washed with petroleum ether (200 mL) and then dried under vacuum to give 3-hydroxy-5-iodo-4H-benzo[e][1,2,4]thiadiazine 1,1-dioxide INT 14A (5.5 g, 8.82 mmol, 39% yield) that was used without further purification. LCMS (m/z) calculated for C₇H₅IN₂O₃S: 323.9; found 322.9 [M−H], t_{R}=0.062 min (Purity Method 2 negative mode).

### Step 14-2. Synthesis of 3-chloro-5-iodo-4H-benzo[e][1,2,4]thiadiazine 1,1-dioxide (INT 14B)

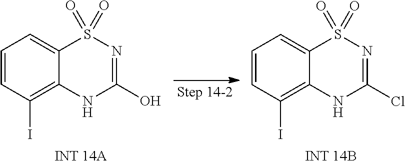 [SMILES: O=S1(=O)N=C(O)Nc2c(I)cccc21 ; O=S1(=O)N=C(Cl)Nc2c(I)cccc21]

To a solution of 3-hydroxy-5-iodo-4H-benzo[e][1,2,4]thiadiazine 1,1-dioxide INT 14A (2.0 g, 6.17 mmol, 1 eq) in POCl₃ (15 mL), N,N-diethylaniline (1.38 g, 9.26 mmol, 1.48 mL, 1.5 eq) was added. The mixture was stirred at 120° C. for 5 h. The solution was cooled to 25° C. and stirred for 15 h. The reaction solution was poured into water (200 mL) portionwise while the temperature was kept below 35° C. The obtained mixture was stirred for 1 h. The dark brown oil eventually formed a brown precipitate that was collected by vacuum filtration and dried under vacuum to give a brown solid. The precipitate was triturated in ethyl acetate (20 ml) and collected by filtration to afford 3-chloro-5-iodo-4H-benzo[e][1,2,4]thiadiazine 1,1-dioxide INT 14B (2.0 g, 4.32 mmol, 70% yield) that was used without further purification. LCMS (m/z) calculated for C7H4ClIN₂O₂S: 341.9; found 340.9 [M−H], t_{R}=0.362 min (Purity Method 2 negative mode).

### Step 14-3. Synthesis of 3-(((3-chloropyridin-2-yl)methyl)amino)-5-iodo-4H-benzo[e][1,2,4]thiadiazine 1,1-dioxide (INT 14C)

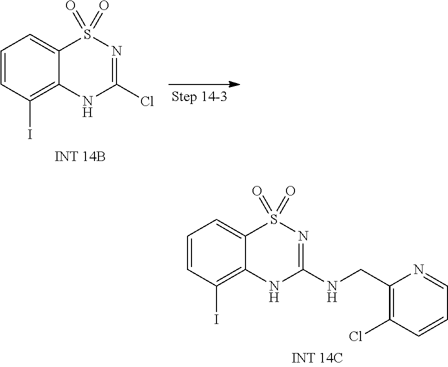 [SMILES: O=S1(=O)N=C(Cl)Nc2c(I)cccc21 ; O=S1(=O)N=C(NCc2ncccc2Cl)Nc2c(I)cccc21]

To a solution of (3-chloro-2-pyridyl)methanamine (616 mg, 4.32 mmol, 1 eq) and 3-chloro-5-iodo-4H-benzo[e][1,2,4]thiadiazine 1,1-dioxide INT 14B (2.0 g, 4.32 mmol, 1 eq) in DMA (10 mL) was added DIPEA (1.68 g, 13.0 mmol, 2.26 mL, 3 eq). The mixture was stirred at 120° C. for 4 h. The reaction mixture was poured into water (40 mL) and extracted with EtOAc (2×30 mL). The combined organics were washed with brine (3×30 mL), dried over Na₂SO₄, filtered, and concentrated in vacuo to give a residue. The residue was purified by flash silica gel chromatography (0-80% Ethyl acetate/petroleum ether gradient at 60 mL/min) to afford 3-(((3-chloropyridin-2-yl)methyl)amino)-5-iodo-4H-benzo[e][1,2,4]thiadiazine 1,1-dioxide INT 14C (750 mg, 1.32 mmol, 31% yield) that was used without further purification. LCMS (m/z) calculated for C₁₃H₁₀ClIN₄O₂S: 447.9; found 449.2 [M+H]⁺, t_{R}=0.445 min (Purity Method 1).

### Step 14-4. Synthesis of (E)-3-(((3-chloropyridin-2-yl)methyl)amino)-5-(2-fluorostyryl)-4H-benzo[e][1,2,4]thiadiazine 1,1-dioxide (14-1)

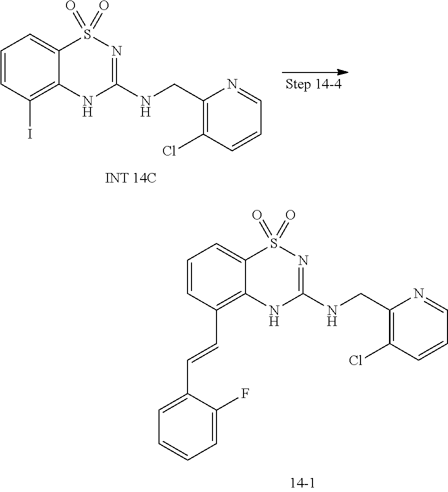 [SMILES: O=S1(=O)N=C(NCc2ncccc2Cl)Nc2c(I)cccc21 ; O=S1(=O)N=C(NCc2ncccc2Cl)Nc2c(/C=C/c3ccccc3F)cccc21]

To a solution of 3-(((3-chloropyridin-2-yl)methyl)amino)-5-iodo-4H-benzo[e][1,2,4]thiadiazine 1,1-dioxide INT 14C (250 mg, 557 mol, 1 eq) and 1-fluoro-2-vinyl-benzene (170 mg, 1.39 mmol, 2.5 eq) in DMF (2 mL) was added Pd(dppf)C12 (82 mg, 111 mol, 0.2 eq) and TEA (113 mg, 1.11 mmol, 155 μL, 2 eq). The mixture was stirred at 120° C. for 14 h. The reaction mixture was poured into water (40 mL) and the product was extracted with EtOAc (2×30 mL). The combined organic phase was washed with brine (3×30 mL), dried over Na₂SO₄, filtered, and concentrated in vacuo to give a residue. The residue was purified by flash silica gel chromatography (0-50% Ethyl acetate/petroleum ether gradient at 60 mL/min) to afford (E)-3-(((3-chloropyridin-2-yl)methyl)amino)-5-(2-fluorostyryl)-4H-benzo[e][1,2,4]thiadiazine 1,1-dioxide 14-1 (15.2 mg, 33 μmol, 6% yield). LCMS (m/z) calculated for C₂₁H16ClFN₄O₂S: 442.1; found 443.2 [M+H]⁺, t_{R}=0.545 min (Purity Method 1). ¹H NMR (400 MHz, DMSO-d6) δ=10.42 (br s, 1H), 8.58 (br d, J=4.3 Hz, 1H), 8.10-8.00 (m, 2H), 7.97-7.86 (m, 2H), 7.68 (d, J=7.4 Hz, 1H), 7.56 (d, J=15.9 Hz, 1H), 7.48-7.38 (m, 2H), 7.37-7.26 (m, 4H), 4.72 (d, J=4.5 Hz, 2H).

### Synthesis of Compound 14-2

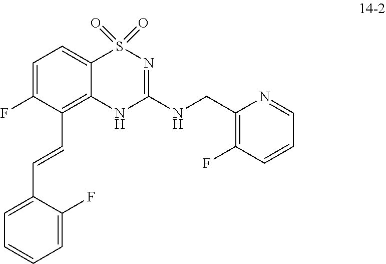 [SMILES: O=S1(=O)N=C(NCc2ncccc2F)Nc2c1ccc(F)c2/C=C/c1ccccc1F]

### Step 14-1B. Synthesis of 5-bromo-6-fluoro-3-(((3-fluoropyridin-2-yl)methyl)amino)-4H-benzo[e][1,2,4]thiadiazine 1,1-dioxide (INT 14-2B)

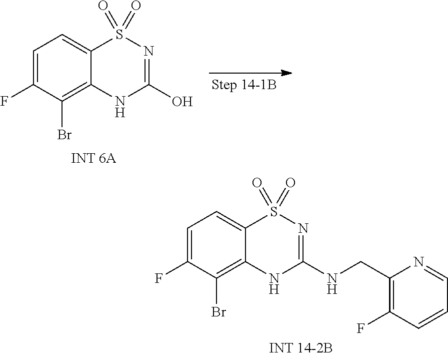 [SMILES: O=S1(=O)N=C(O)Nc2c1ccc(F)c2Br ; O=S1(=O)N=C(NCc2ncccc2F)Nc2c1ccc(F)c2Br]

To a solution of (3-fluoro-2-pyridyl)methanamine (855 mg, 4.29 mmol, 1.27 eq, 2HCl) in MeCN (20 mL) was added BOP (2.25 g, 5.08 mmol, 1.5 eq) and DBU (2.06 g, 13.56 mmol 2.04 mL, 4 eq) at 0° C. The mixture was stirred at 0° C. for 1 h. 5-bromo-6-fluoro-3-hydroxy-4H-benzo[e][1,2,4]thiadiazine 1,1-dioxide INT 6A, which was prepared in an analogous fashion to INT 14A (1.0 g, 3.39 mmol, 1 eq) was added. The mixture was stirred at 80° C. for 14 h. The reaction mixture was poured into water (40 mL). The product was extracted with EtOAc (2×30 mL). The combined organics were washed with brine (3×30 mL), dried over Na₂SO₄, filtered, and concentrated in vacuo to give a residue. The residue was purified by flash silica gel chromatography (Eluent of 0˜80% ethyl acetate/petroleum ether gradient at 60 mL/min) to afford 5-bromo-6-fluoro-3-(((3-fluoropyridin-2-yl)methyl)amino)-4H-benzo[e][1,2,4]thiadiazine 1,1-dioxide (INT 14-2B) (800 mg, 1.71 mmol, 50% yield) that was used without further purification. LCMS (m/z) calculated for C₁₃H₉BrF₂N₄O₂S: 402.0; found 403.0 [M+H]⁺, t_{R}=0.428 min (Purity Method 1).

### Step 14-2B. Synthesis of (E)-9-(2-fluorostyryl)-9-borabicyclo[3.3.1]nonane (INT 14-2C)

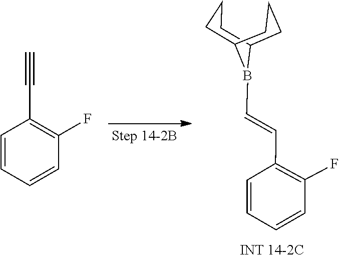

To a solution of 1-ethynyl-2-fluoro-benzene (0.2 g, 1.66 mmol, 189 μL, 1 eq) in THF (10 mL), was added 9-borabicyclo[3.3.1]nonane (0.5 M, 10 mL, 3.00 eq) at 0° C. The mixture was stirred at 25° C. for 16h to afford (E)-9-(2-fluorostyryl)-9-borabicyclo[3.3.1]nonane INT 14-2C that was used as a crude solution without purification.

### Step 14-3B. Synthesis of (E)-6-fluoro-3-(((3-fluoropyridin-2-yl)methyl)amino)-5-(2-fluorostyryl)-4H-benzo[e][1,2,4]thiadiazine 1,1-dioxide (14-2)

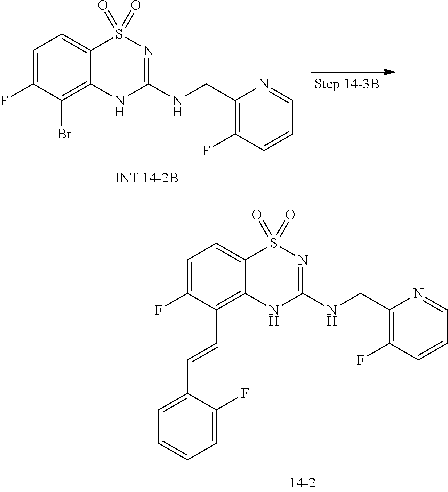 [SMILES: O=S1(=O)N=C(NCc2ncccc2F)Nc2c1ccc(F)c2Br ; O=S1(=O)N=C(NCc2ncccc2F)Nc2c1ccc(F)c2/C=C/c1ccccc1F]

To a solution of 9-[(E)-2-(2-fluorophenyl)vinyl]-9-borabicyclo[3.3.1]nonane INT 14-2C (0.4 g, 1.65 mmol, 3.33 eq) and 5-bromo-6-fluoro-N-[(3-fluoro-2-pyridyl)methyl]-1,1-dioxo-4H-1,2,4-benzothiadiazin-3-amine INT 14-2B (200 mg, 496 mol, 1 eq) in THE (30 mL) and H₂O (4 mL) were added K₃PO₄ (632 mg, 2.98 mmol, 6 eq) and [2-(2-aminophenyl)phenyl]-chloro-palladium tri-tert-butylphosphane (127 mg, 248 mol, 0.5 eq). The mixture was stirred at 60° C. for 14 h. The reaction mixture was poured into water (40 mL). The product was extracted with EtOAc (2×30 mL). The combined organics were washed with brine (3×30 mL), dried over Na₂SO₄, filtered, and concentrated in vacuo to give a residue. The residue was purified by flash silica gel chromatography (Eluent of 0-100% Ethyl acetate/Petroleum ether gradient at 80 mL/min) and then repurified by preparative HPLC (column: C18 150×30 mm; A-B mobile phase: [water(FA)-ACN]; gradient:488%-78% B over 7 min) and lyophilized to afford (E)-6-fluoro-3-(((3-fluoropyridin-2-yl)methyl)amino)-5-(2-fluorostyryl)-4H-benzo[e][1,2,4]thiadiazine 1,1-dioxide 14-2 (35 mg, 78 mol, 16% yield). LCMS (m/z) calculated for C₂₁H₁₅F₃N₄O₂S: 444.1; found 445.1 [M+H]⁺, t_{R}=0.534 min (Purity Method 1). ¹H NMR (400 MHz, DMSO-d6) δ=10.37-10.24 (m, 1H), 8.50-8.38 (m, 1H), 8.15 (br t, J=4.6 Hz, 1H), 7.95 (br t, J=7.2 Hz, 1H), 7.79 (ddd, J=1.2, 8.5, 9.9 Hz, 1H), 7.73 (dd, J=5.6, 8.7 Hz, 1H), 7.54-7.40 (m, 2H), 7.38-7.28 (m, 3H), 7.27-7.19 (m, 2H), 4.70 (br d, J=4.0 Hz, 2H).

### Synthesis of Scheme 14 Compounds

Alternatively for Step 14-3B, 14-3 was synthesized using 1.4 eq of 1-fluoro-2-vinyl-benzene, 0.14 eq of [2-(2-aminophenyl)phenyl]-chloro-palladium;tritert-butylphosphane, and 2 eq of dicyclohexylamine in dioxanes at 100° C. for 15h.

The compounds listed in Table 15 were made using the procedures of Scheme 14.

TABLE 15

| | | Purity | Exact | | | |
|---|---|---|---|---|---|---|
| Cpd | | RT | mass | observed | | Purity |
| No. | Structure | (min) | (g/mmol) | m/z | Ion | Method |
| 14-1 | 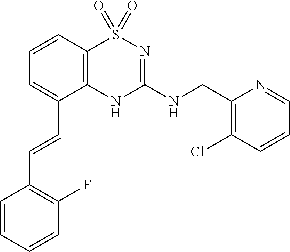 [SMILES: O=S1(=O)N=C(NCc2ncccc2Cl)Nc2c(/C=C/c3ccccc3F)cccc21] | 0.545 | 442.1 | 443.2 | [M + H]⁺ | 1 |
| 14-2 | 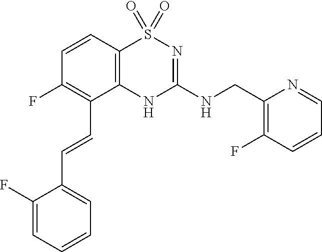 [SMILES: O=S1(=O)N=C(NCc2ncccc2F)Nc2c1ccc(F)c2/C=C/c1ccccc1F] | 0.534 | 444.1 | 445.1 | [M + H]⁺ | 1 |
| 14-3 | 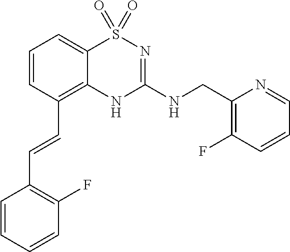 [SMILES: O=S1(=O)N=C(NCc2ncccc2F)Nc2c(/C=C/c3ccccc3F)cccc21] | 0.530 | 426.1 | 427.0 | [M + H]⁺ | 1 |

### Example 15

### Synthesis of Representative Compound 15-1

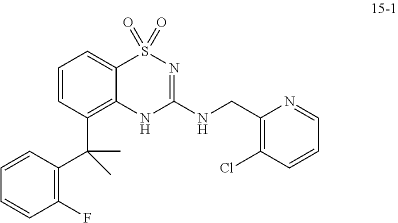 [SMILES: CC(C)(c1ccccc1F)c1cccc2c1NC(NCc1ncccc1Cl)=NS2(=O)=O]

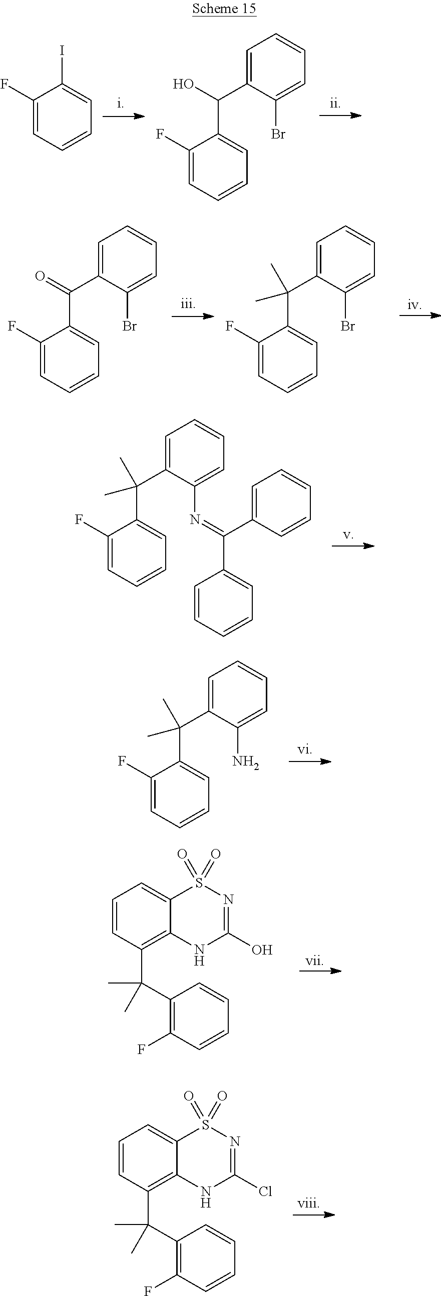

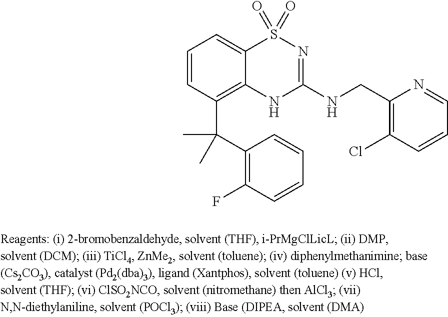 [SMILES: CC(C)(c1ccccc1F)c1cccc2c1NC(NCc1ncccc1Cl)=NS2(=O)=O]

### Step 15-1. Synthesis of (7-bromophenyl)(2-fluorophenyl)methanol (INT 15A)

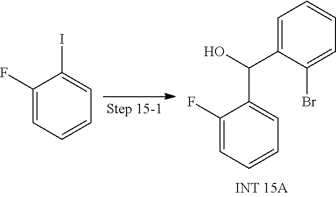 [SMILES: Fc1ccccc1I ; OC(c1ccccc1F)c1ccccc1Br]

To a solution of 1-fluoro-2-iodo-benzene (7.0 g, 32 mmol, 3.68 mL, 1 eq) in THE (50 mL) was added i-PrMgCl·LiCl (1.3 M, 24.3 mL, 1 eq). The mixture was stirred at 0° C. for 1 h. Then, 2-bromobenzaldehyde (5.83 g, 31.5 mmol, 3.65 mL, 1 eq) was added to the reaction mixture at 25° C. and stirred for 2 h. The reaction mixture was quenched with slow addition of aq. sat. NH₄Cl solution (100 mL) while stirring. EtOAc (100 mL) was added and the layers were separated. The aqueous phase was extracted with EtOAc (2×100 mL). Combined organic layers were dried over Na₂SO₄, filtered, and concentrated in vacuo to give a residue. The residue was purified by flash silica gel chromatography (0-8% Ethyl acetate/petroleum ether gradient at 60 mL/min) to afford (2-bromophenyl)(2-fluorophenyl)methanol INT 15A (8.0 g, 18.5 mmol, 59% yield) that was used without further purification. ¹H NMR (400 MHz, DMSO-d6) δ=7.62 (dd, J=1.4, 7.8 Hz, 1H), 7.57 (dd, J=0.9, 8.0 Hz, 1H), 7.43 (t, J=7.2 Hz, 1H), 7.31 (ddt, J=1.9, 5.5, 7.6 Hz, 1H), 7.26-7.11 (m, 4H), 6.20-6.10 (m, 2H).

### Step 15-2. Synthesis of (2-bromophenyl)(2-fluorophenyl)methanone (INT 15B)

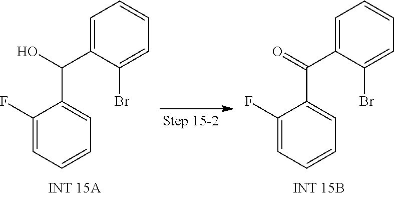 [SMILES: OC(c1ccccc1F)c1ccccc1Br ; O=C(c1ccccc1F)c1ccccc1Br]

To a solution of (2-bromophenyl)-(2-fluorophenyl)methanol INT 15A (8.0 g, 18.50 mmol, 65% purity, 1 eq) in DCM (50 mL), was added DMP (15.69 g, 36.99 mmol, 11.45 mL, 2 eq). The mixture was stirred at 25° C. for 2 h. The reaction solution was cooled to room temperature and then diluted with aq. sat. NaHCO₃ solution (100 mL), then Na₂SO₃ (15 g) was added under stirring. The aqueous phase was extracted with ethyl acetate (3×200 mL). The combined organic layers were washed with brine (200 mL), dried over anhydrous sodium sulfate, filtered, and concentrated in vacuo to give a brown oil. The residue was purified by flash silica gel chromatography (Eluent of 0˜8% Ethyl acetate/Petroleum ether gradient at 80 mL/min) to afford (2-bromophenyl)(2-fluorophenyl)methanone INT 15B (6.0 g, 17.4 mmol, 94% yield) that was used without further purification. LCMS (m/z) calculated for C₁₃H₈BrFO: 278.0; found 279.0 [M+H]⁺, t_{R}=0.660 min (Purity Method 1). ¹H NMR (400 MHz, DMSO-d6) δ=7.77-7.65 (m, 3H), 7.56-7.44 (m, 3H), 7.42-7.27 (m, 2H).

### Step 15-3. Synthesis of 1-bromo-2-(2-(2-fluorophenyl)propan-2-yl)benzene (INT 15C)

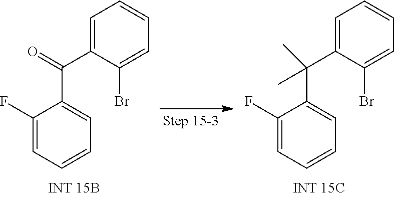 [SMILES: O=C(c1ccccc1F)c1ccccc1Br ; CC(C)(c1ccccc1F)c1ccccc1Br]

TiCl₄ (8.16 g, 43.0 mmol, 4.80 mL, 3 eq) and toluene (50 mL) were added to a dried flask under N₂. The reaction was cooled to −40° C. with stirring, and ZnMe₂ (1 M, 42.99 mL, 3 eq) was added dropwise. The reaction was stirred for 30 min. A solution of (2-bromophenyl)-(2-fluorophenyl)methanone INT 15B (4.0 g, 14.3 mmol, 1 eq) in toluene (20 mL) was added dropwise to the above solution. The reaction was stirred for 3.5 h at −40° C. and stirred at 25° C. for 12 h. The reaction mixture was quenched with slow addition to water (300 mL) under stirring at 0° C. EtOAc (300 mL) was added and the layers were separated. The aqueous phase was extracted with EtOAc (2×200 mL). Combined organics were dried over Na₂SO₄, filtered, and concentrated in vacuo to give a residue. The residue was purified by flash silica gel chromatography (Eluent of 0˜1% Ethyl acetate/Petroleum ether gradient at 80 mL/min) to afford 1-bromo-2-(2-(2-fluorophenyl)propan-2-yl)benzene INT 15C (3.0 g, 5.73 mmol, 40% yield) that was used without further purification. ¹H NMR (400 MHz, DMSO-d6) δ=7.69 (dd, J=1.6, 8.0 Hz, 1H), 7.51-7.38 (m, 5H), 7.31-7.20 (m, 2H), 6.97 (ddd, J=1.4, 8.0, 12.4 Hz, 1H), 5.83 (t, J=1.0 Hz, 1H), 5.50 (s, 1H), 1.73 (s, 6H).

### Step 15-4. Synthesis of N-(2-(2-(2-fluorophenyl)propan-2-yl)phenyl)-1,1-diphenylmethanimine (INT 15D)

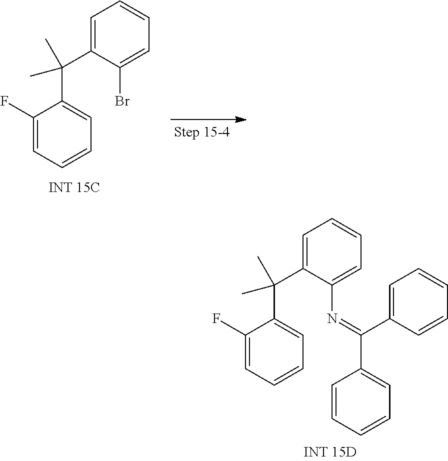 [SMILES: CC(C)(c1ccccc1F)c1ccccc1Br ; CC(C)(c1ccccc1F)c1ccccc1N=C(c1ccccc1)c1ccccc1]

A mixture of 1-bromo-2-[1-(2-fluorophenyl)-1-methyl-ethyl]benzene INT 15C (3.0 g, 10.2 mmol, 1 eq), Cs₂CO₃ (10.00 g, 30.70 mmol, 3 eq), diphenylmethanimine (3.71 g, 20.5 mmol, 3.43 mL, 2 eq), Pd₂(dba)₃ (937 mg, 1.02 mmol, 0.1 eq), Xantphos (1.18 g, 2.05 mmol, 0.2 eq) in toluene (3 mL) was degassed and purged with N₂ 3 times, and then the mixture was stirred at 100° C. for 20 h under an N₂ atmosphere. Additional Pd₂(dba)₃ (100 mg, 109 mol, 1.07e-2 eq) and Xantphos (150 mg, 259.24 mol, 2.53e-2 eq) were added, and the mixture was stirred at 100° C. for another 14 h under an N₂ atmosphere. The crude mixture was filtered through a pad of celite and the filtrate was concentrated in vacuo to give a residue. The residue was purified by flash silica gel chromatography (0˜1% Ethyl acetate/Petroleum ether gradient at 60 mL/min) to afford N-(2-(2-(2-fluorophenyl)propan-2-yl)phenyl)-1,1-diphenylmethanimine INT 15D (1.1 g, 2.54 mmol, 25% yield). ¹H NMR (400 MHz, DMSO-d6) δ=7.49-7.44 (m, 2H), 7.37 (t, J=7.6 Hz, 2H), 7.30-7.16 (m, 6H), 7.08-7.00 (m, 1H), 6.99-6.90 (m, 2H), 6.81 (dt, J=1.3, 7.6 Hz, 1H), 6.75 (dd, J=1.4, 7.9 Hz, 2H), 6.64 (dt, J=1.3, 7.6 Hz, 1H), 6.07 (dd, J=1.3, 7.8 Hz, 1H), 1.80 (s, 6H).

### Step 15-5. Synthesis of 2-(2-(2-fluorophenyl)propan-2-yl)aniline (INT 15E)

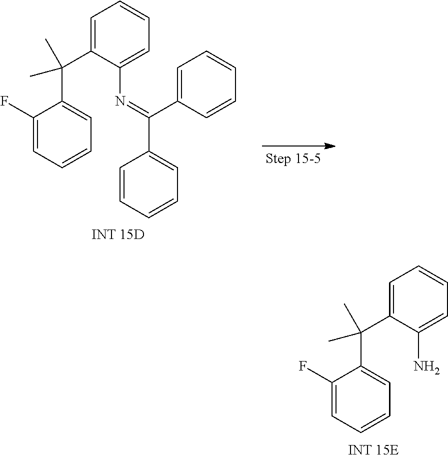 [SMILES: CC(C)(c1ccccc1F)c1ccccc1N=C(c1ccccc1)c1ccccc1 ; CC(C)(c1ccccc1N)c1ccccc1F]

To a solution of N-(2-(2-(2-fluorophenyl)propan-2-yl)phenyl)-1,1-diphenylmethanimine INT 15D (1.1 g, 2.80 mmol, 1 eq) in THE (10 mL), was added HCl (2 M, 13.98 mL, 10 eq). The mixture was stirred at 25° C. for 0.5h. The reaction mixture was poured into water (40 mL). The aqueous layer was extracted with EtOAc (2×30 mL). The combined organics were washed with brine (3×30 mL), dried over Na₂SO₄, filtered, and concentrated in vacuo to give a residue. The residue was purified by flash silica gel chromatography (0-3% Ethyl acetate/petroleum ether gradient at 50 mL/min) to afford 2-(2-(2-fluorophenyl)propan-2-yl)aniline INT 15E (600 mg, 2.59 mmol, 93% yield). LCMS (m/z) calculated for C₁₅H₁₆FN: 229.1; found 230.2 [M+H]⁺, t_{R}=0.553 min (Purity Method 1). ¹H NMR (400 MHz, DMSO-d6) δ=7.56-7.49 (m, 1H), 7.34-7.26 (m, 2H), 7.25-7.19 (m, 1H), 7.03 (ddd, J=1.3, 8.0, 12.3 Hz, 1H), 6.97-6.89 (m, 1H), 6.64 (dt, J=1.3, 7.5 Hz, 1H), 6.52 (dd, J=1.1, 7.9 Hz, 1H), 3.90 (s, 2H), 1.65 (s, 6H).

### Step 15-6. Synthesis of 5-(2-(2-fluorophenyl)propan-2-yl)-3-hydroxy-4H-benzo[e][1,2,4]thiadiazine 1,1-dioxide (INT 15F)

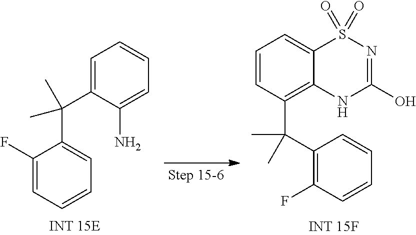 [SMILES: CC(C)(c1ccccc1N)c1ccccc1F ; CC(C)(c1ccccc1F)c1cccc2c1NC(O)=NS2(=O)=O]

To a solution of N-(oxomethylene)sulfamoyl chloride (813 mg, 5.74 mmol, 500 μL, 2.63 eq) in nitrobenzene (3 mL) was added 2-(2-(2-fluorophenyl)propan-2-yl)aniline INT 15E (500 mg, 2.18 mmol, 1 eq) at 0° C. The mixture was stirred at 0° C. for 0.5 h then AlCl₃ (500 mg, 3.75 mmol, 1.72 eq) was added. The mixture was stirred at 120° C. for 1.5 h. The reaction solution was cooled to room temperature and then diluted with saturated NH₄Cl aqueous solution (50 mL). The aqueous layer was extracted with ethyl acetate (3×50 mL). The combined organic layers were washed with brine (80 mL), dried over anhydrous sodium sulfate, filtered, and concentrated in vacuo to give a brown oil. The obtained brown oil was diluted with petroleum ether (100 mL) and then stirred for 10 min. The brown oil eventually formed a brown precipitate that was collected by vacuum filtration. The filter cake was washed with petroleum ether (100 mL) and then dried under vacuum to afford 5-(2-(2-fluorophenyl)propan-2-yl)-3-hydroxy-4H-benzo[e][1,2,4]thiadiazine 1,1-dioxide INT 15F (500 mg, 1.05 mmol, 48% yield). LCMS (m/z) calculated for C₁₆H15FN₂O₃S: 334.1; found 333.1 [M−H], t_{R}=0.478 min (Purity Method 2 negative mode). ¹H NMR (400 MHz, DMSO-d6) δ=7.86 (br d, J=8.1 Hz, 1H), 7.77-7.64 (m, 2H), 7.37 (br s, 3H), 6.97 (br s, 1H), 1.72 (s, 6H).

### Step 15-7. Synthesis of 3-chloro-5-(2-(2-fluorophenyl)propan-2-yl)-4H-benzo[e][1,2,4]thiadiazine 1,1-dioxide (INT 15G)

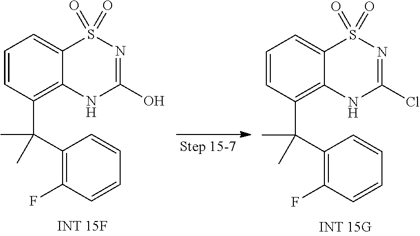 [SMILES: CC(C)(c1ccccc1F)c1cccc2c1NC(O)=NS2(=O)=O ; CC(C)(c1ccccc1F)c1cccc2c1NC(Cl)=NS2(=O)=O]

To a solution of 5-[1-(2-fluorophenyl)-1-methyl-ethyl]-1,1-dioxo-4H-1,2,4-benzothiadiazin-3-ol INT 15F (300 mg, 897 mol, 1 eq) in POCl₃ (2 mL) was added N,N-diethylaniline (280 mg, 1.88 mmol, 0.3 mL, 2.09 eq). The mixture was stirred at 120° C. for 2 h. The reaction solution was poured into water (80 mL) portionwise while the temperature was kept below 35° C. The obtained mixture was stirred for 1 h. The mixture was extracted with EtOAc (3×80 mL). The combined organics were washed with brine (2×60 mL), dried over Na₂SO₄, filtered, and concentrated in vacuo to afford a residue. The residue was purified by prep-TLC (SiO₂, PE: EtOAc=1:3, Rf=0.40) to afford 3-chloro-5-(2-(2-fluorophenyl)propan-2-yl)-4H-benzo[e][1,2,4]thiadiazine 1,1-dioxide INT 15G (125 mg, 294 mol, 33% yield) that was used without further purification. LCMS (m/z) calculated for C₁₆H14ClFN₂O₂S: 352.0; found 351.1 [M−H], t_{R}=0.412 min (Purity Method 2 negative mode).

### Step 15-8. Synthesis of 3-(((3-chloropyridin-2-yl)methyl)amino)-5-(2-(2-fluorophenyl)propan-2-yl)-4H-benzo[e][1,2,4]thiadiazine 1,1-dioxide (15-1)

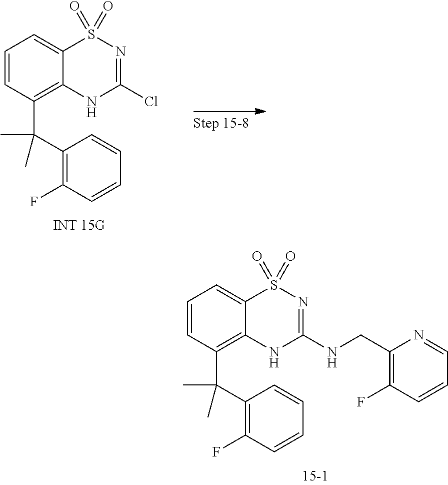 [SMILES: CC(C)(c1ccccc1F)c1cccc2c1NC(Cl)=NS2(=O)=O ; CC(C)(c1ccccc1F)c1cccc2c1NC(NCc1ncccc1F)=NS2(=O)=O]

To a solution of (3-chloro-2-pyridyl)methanamine (126 mg, 882 mol, 3 eq) in DMA (1 mL), was added DIPEA (114 mg, 882 mol, 154 μL, 3 eq). The mixture was stirred at 25° C. for 14 h. The reaction mixture was poured into water (30 mL). The mixture was extracted with EtOAc (2×30 mL). The combined organics were washed with brine (3×30 mL), dried over Na₂SO₄, filtered, and concentrated in vacuo to give a residue. The residue was purified by preparative HPLC (column: Phenomenex Luna C18 150×25 mm×10 um; A-B mobile phase: [water(TFA)-ACN]; gradient: 45%-75% B over 9 min) and lyophilized to afford 3-(((3-chloropyridin-2-yl)methyl)amino)-5-(2-(2-fluorophenyl)propan-2-yl)-4H-benzo[e][1,2,4]-thiadiazine 1,1-dioxide 15-1 (44 mg, 95.87 mol, 33% yield). LCMS (m/z) calculated for C₂₂H₂₀ClFN₄O₂S: 458.1; found 459.3 [M+H]⁺, t_{R}=0.531 min (Purity Method 1). ¹H NMR (400 MHz, DMSO-d6) δ=8.52 (dd, J=0.9, 4.6 Hz, 1H), 8.31 (br s, 1H), 7.97 (br d, J=7.8 Hz, 1H), 7.77 (br d, J=7.8 Hz, 2H), 7.67 (d, J=7.6 Hz, 1H), 7.52-7.01 (m, 6H), 4.80-4.20 (m, 2H), 2.07 (s, 1H), 1.79 (br s, 6H).

### Synthesis of Scheme 15 Compounds

The compounds listed in Table 16 were made using the procedures of Scheme 15.

TABLE 16

| | | Purity | Exact | | | |
|---|---|---|---|---|---|---|
| Cpd | | RT | mass | observed | | Purity |
| No. | Structure | (min) | (g/mol) | m/z | Ion | Method |
| 15-1 | 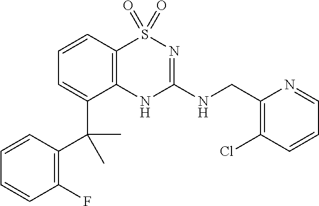 [SMILES: CC(C)(c1ccccc1F)c1cccc2c1NC(NCc1ncccc1Cl)=NS2(=O)=O] | 0.531 | 458.1 | 459.3 | [M + H]⁺ | 1 |

### Example 16

### Synthesis of Representative Compound 16-1

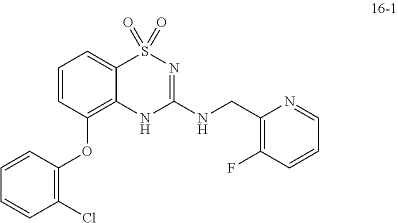 [SMILES: O=S1(=O)N=C(NCc2ncccc2F)Nc2c(Oc3ccccc3Cl)cccc21]

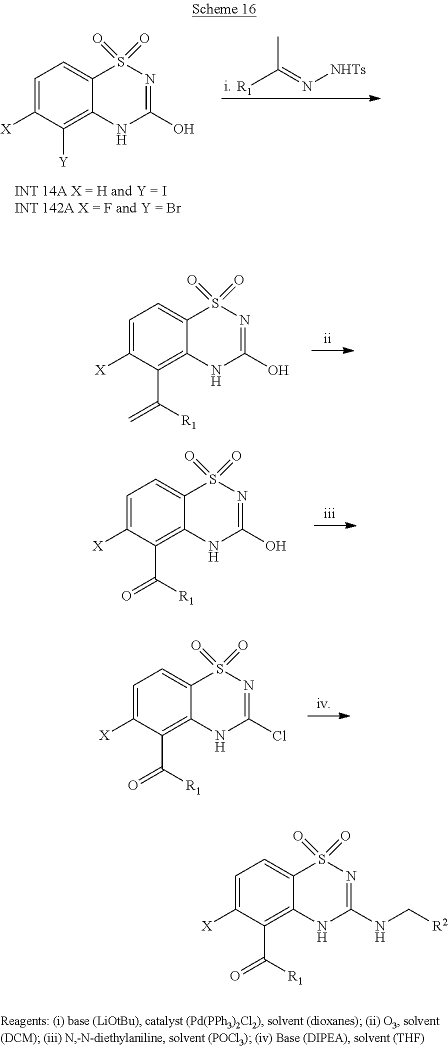

### Step 16-1. Synthesis of (E)-N′-(1-(2-chlorophenyl)ethylidene)-4-methylbenzenesulfonohydrazide (INT 16A)

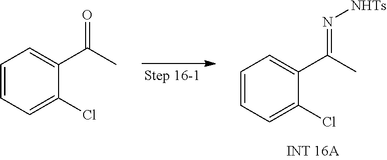

To a solution of 1-(2-chlorophenyl)ethanone (5.0 g, 32.3 mmol, 4.21 mL, 1 eq) in MeOH (20 mL) was added 4-methylbenzenesulfonohydrazide (7.23 g, 38.8 mmol, 1.2 eq). The mixture was stirred at 60° C. for 6 h. The white precipitate was collected by filtration, air-dried, triturated in MeOH (20 mL) and collected by filtration to afford (E)-N′-(1-(2-chlorophenyl)ethylidene)-4-methylbenzenesulfonohydrazide INT 16A (8.1 g, 24.1 mmol, 74% yield). ¹H NMR (400 MHz, DMSO-d6) δ=10.67 (s, 1H), 7.74 (d, J=8.3 Hz, 2H), 7.47-7.30 (m, 5H), 7.17 (dd, J=1.9, 7.4 Hz, 1H), 2.37 (s, 3H), 2.16 (s, 3H).

### Step 16-2. Synthesis of 5-(1-(2-chlorophenyl)vinyl)-3-hydroxy-4H-benzo[e][1,2,4]thiadiazine 1,1-dioxide (INT 16B)

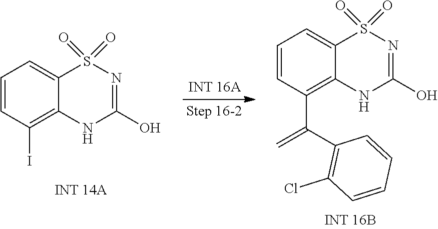 [SMILES: O=S1(=O)N=C(O)Nc2c(I)cccc21 ; C=C(c1ccccc1Cl)c1cccc2c1NC(O)=NS2(=O)=O]

To a solution of N-[(E)-1-(2-chlorophenyl)ethylideneamino]-4-methyl-benzenesulfonamide INT 16A (4.78 g, 14.8 mmol, 1.2 eq) and 5-iodo-1,1-dioxo-4H-1,2,4-benzothiadiazin-3-ol INT 14A (4.0 g, 12.3 mmol, 1 eq) in dioxane (30 mL) were added tBuOLi (2.96 g, 37.0 mmol, 3.34 mL, 3 eq) and Pd(PPh₃)₂Cl₂ (1.73 g, 2.47 mmol, 0.2 eq). The mixture was stirred at 100° C. for 14 h. The reaction mixture was poured into water (80 mL). The mixture was extracted with EtOAc (2×80 mL). The combined organics were washed by brine (3×80 mL), dried over Na₂SO₄, filtered, and concentrated in vacuo to give crude product (2.3 g, 6.87 mmol, 56% yield). The crude product was used directly in the next step without further purification. LCMS (m/z) calculated for C₁₅H11ClIN₂O₃S: 334.0; found 333.0 [M−H], t_{R}=0.462 min (Purity Method 2 negative mode).

### Step 16-3. Synthesis of (2-chlorophenyl)(3-hydroxy-1,1-dioxido-4H-benzo[e][1,2,4]thiadiazin-5-yl)methanone (INT 16C)

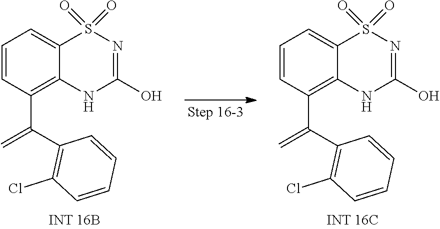 [SMILES: C=C(c1ccccc1Cl)c1cccc2c1NC(O)=NS2(=O)=O ; C=C(c1ccccc1Cl)c1cccc2c1NC(O)=NS2(=O)=O]

A dry 100-mL round-bottomed flask containing a magnetic stirring bar was charged with 5-[1-(2-chlorophenyl)vinyl]-1,1-dioxo-4H-1,2,4-benzothiadiazin-3-ol INT 16B (2.3 g, 6.87 mmol, 1 eq) and DCM (80 mL). A gentle stream of dry ozone was passed through the solution and the flask was immediately cooled to −78° C. Ozonolysis was continued until the distinctive blue color of excess ozone was first observed. Ozonolysis was then terminated, and the excess ozone was removed by purging with a stream of nitrogen for 5-10 min. The solution was allowed to warm to 25° C., and Me₂S (5.92 g, 95.31 mmol, 7 mL, 13.87 eq) was added via syringe. The solution was allowed to stir at 25° C. for 16 h. The reaction mixture was concentrated in vacuo to give a crude residue. The residue was purified by flash silica gel chromatography (Eluent of 0-50% Ethyl acetate/Petroleum ether) to afford (2-chlorophenyl)(3-hydroxy-1,1-dioxido-4H-benzo[e][1,2,4]thiadiazin-5-yl)methanone INT 16C (1.4 g, 3.45 mmol, 50% yield).

### Step 16-4. Synthesis of (3-chloro-1,1-dioxido-4H-benzo[e][1,2,4]thiadiazin-5-yl)(2-chlorophenyl)methanone (INT 16D)

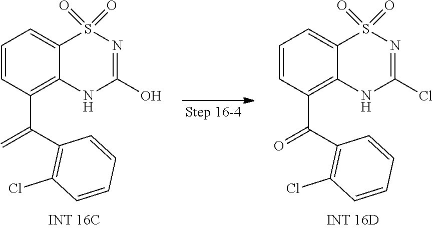 [SMILES: C=C(c1ccccc1Cl)c1cccc2c1NC(O)=NS2(=O)=O ; O=C(c1ccccc1Cl)c1cccc2c1NC(Cl)=NS2(=O)=O]

To a solution of (2-chlorophenyl)(3-hydroxy-1,1-dioxido-4H-benzo[e][1,2,4]thiadiazin-5-yl)methanone INT 16C (700 mg, 2.08 mmol, 1 eq) in POCl₃ (10 mL) was added N,N-diethylaniline (653 mg, 4.38 mmol, 0.7 mL, 2.11 eq). The mixture was stirred at 120° C. for 2 h. The reaction solution was poured into water (80 mL) portionwise while the temperature was kept below 35° C. The obtained mixture was stirred for 1 h. The dark brown oil eventually formed a brown precipitate that was collected by vacuum filtration and dried under vacuum to give (3-chloro-1,1-dioxido-4H-benzo[e][1,2,4]thiadiazin-5-yl)(2-chlorophenyl)methanone INT 16D (200 mg, 544 mol, 26% yield). LCMS (m/z) calculated for C14H18C12N₂O₃S: 354.0; found 353.0 [M−H], t_{R}=0.320 min (Purity Method 2 negative mode).

### Step 16-5. Synthesis of (2-chlorophenyl)(3-(((3-chloropyridin-2-yl)methyl)amino)-1,1-dioxido-4H-benzo[e][1,2,4]thiadiazin-5-yl)methanone (16-1)

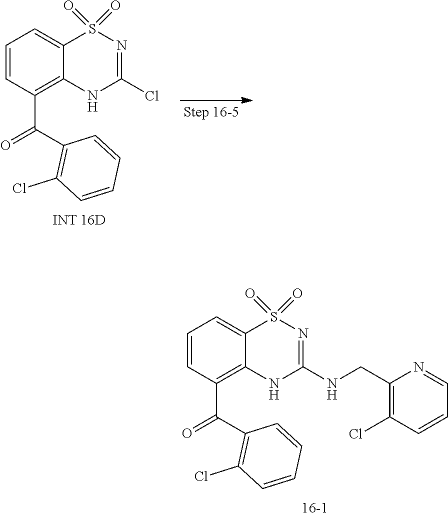 [SMILES: O=C(c1ccccc1Cl)c1cccc2c1NC(Cl)=NS2(=O)=O ; O=C(c1ccccc1Cl)c1cccc2c1NC(NCc1ncccc1Cl)=NS2(=O)=O]

To a solution of (3-chloro-2-pyridyl)methanamine (42.2 mg, 296 mol, 1.5 eq) and (3-chloro-1,1-dioxido-4H-benzo[e][1,2,4]thiadiazin-5-yl)(2-chlorophenyl)methanone INT 16D (70 mg, 197 mol, 1 eq) in THE (1.5 mL) was added DIPEA (76.4 mg, 591 mol, 103 μL, 3 eq). The mixture was stirred at 25° C. for 16 h. The reaction mixture was concentrated in vacuo to give a residue. The residue was purified by preparative HPLC (column: Phenomenex luna C18 150×25 mm×10 um; A-B mobile phase: [water(FA)-ACN]; gradient: 50%-70% B over 10 min) and lyophilized to afford (2-chlorophenyl)(3-(((3-chloropyridin-2-yl)methyl)amino)-1,1-dioxido-4H-benzo[e][1,2,4]thiadiazin-5-yl)methanone 16-1 (18 mg, 39 mol, 20% yield). LCMS (m/z) calculated for C₂₀H₁₄Cl₂N₄O₃S: 460.0; found 461.1 [M+H]⁺, t_{R}=0.516 min (Purity Method 1). ¹H NMR (400 MHz, DMSO-d6) δ=11.53 (br s, 1H), 9.39 (br d, J=3.6 Hz, 1H), 8.59-8.53 (m, 1H), 8.10-8.05 (m, 1H), 8.00 (d, J=8.0 Hz, 1H), 7.72-7.60 (m, 3H), 7.60-7.51 (m, 2H), 7.44 (dd, J=4.6, 7.9 Hz, 1H), 7.35 (t, J=7.9 Hz, 1H), 4.74 (br d, J=5.0 Hz, 2H).

The compounds listed in Table 17 were made using the procedures of Scheme 16.

TABLE 17

| | | Purity | Exact | | | |
|---|---|---|---|---|---|---|
| Cpd | | RT | mass | observed | | Purity |
| No. | Structure | (min) | (g/mol) | m/z | Ion | Method |
| 16-1 | 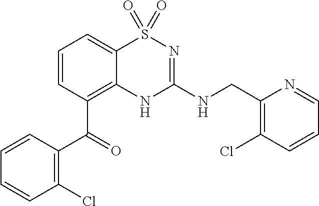 [SMILES: O=C(c1ccccc1Cl)c1cccc2c1NC(NCc1ncccc1Cl)=NS2(=O)=O] | 0.516 | 460.0 | 461.1 | [M + H]⁺ | 1 |
| 16-2 | 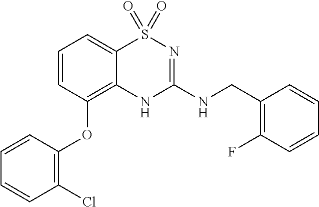 [SMILES: O=S1(=O)N=C(NCc2ccccc2F)Nc2c(Oc3ccccc3Cl)cccc21] | 0.540 | 443.1 | 444.1 | [M + H]⁺ | 1 |
| 16-3 | 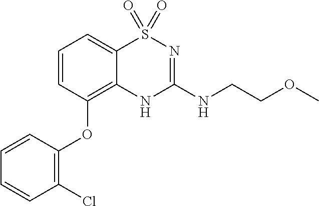 | 0.486 | 393.1 | 394.1 | [M + H]⁺ | 1 |
| 16-4 | 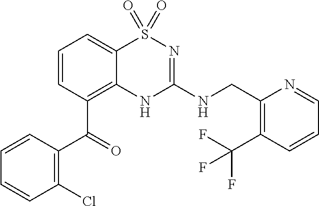 [SMILES: O=C(c1ccccc1Cl)c1cccc2c1NC(NCc1ncccc1C(F)(F)F)=NS2(=O)=O] | 0.535 | 494.0 | 495.0 | [M + H]⁺ | 1 |
| 16-5 | 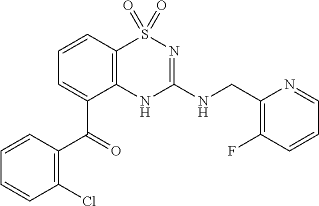 [SMILES: O=C(c1ccccc1Cl)c1cccc2c1NC(NCc1ncccc1F)=NS2(=O)=O] | 0.497 | 444.1 | 445.1 | [M + H]⁺ | 1 |
| 16-6 | 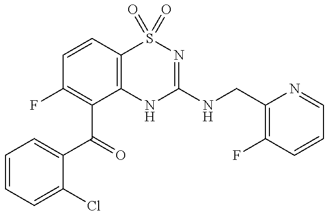 [SMILES: O=C(c1ccccc1Cl)c1c(F)ccc2c1NC(NCc1ncccc1F)=NS2(=O)=O] | 0.503 | 462.0 | 463.0 | [M + H]⁺ | 1 |

### Example 17

### Synthesis of Representative Compound 17-1

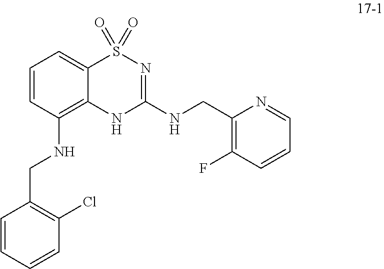 [SMILES: O=S1(=O)N=C(NCc2ncccc2F)Nc2c(NCc3ccccc3Cl)cccc21]

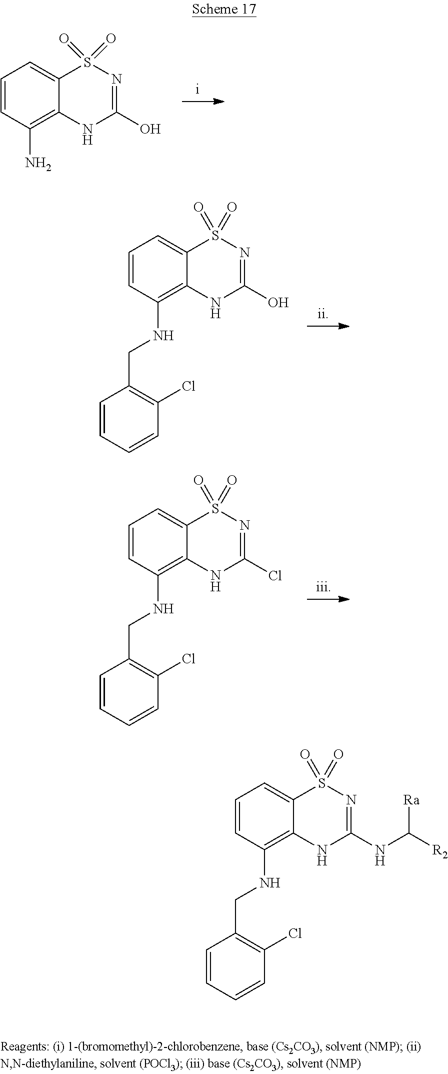

### Step 17-1. Synthesis of 5-((2-chlorobenzyl)amino)-3-hydroxy-4H-benzo[e][1,2,4]thiadiazine 1,1-dioxide (INT 17A)

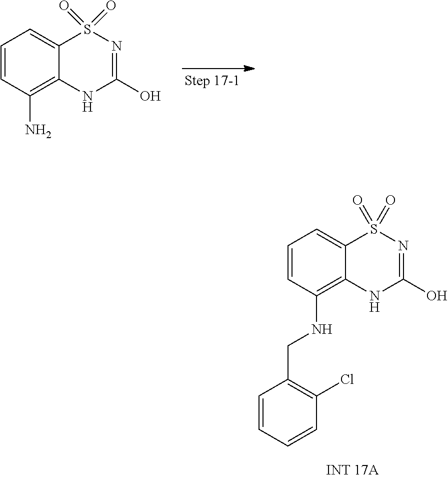 [SMILES: Nc1cccc2c1NC(O)=NS2(=O)=O ; O=S1(=O)N=C(O)Nc2c(NCc3ccccc3Cl)cccc21]

To a stirring solution of 5-amino-3-hydroxy-4H-benzo[e][1,2,4]thiadiazine 1,1-dioxide (100 mg, 1 Eq, 469 mol) in NMP (5 mL) was added 1-(bromomethyl)-2-chlorobenzene (125 mg, 1.3 Eq, 610 mol) and cesium carbonate (306 mg, 2 Eq, 938 mol). The mixture was heated to 60° C. and stirred for 18 h. The reaction mixture was purified by HPLC to obtain 5-((2-chlorobenzyl)amino)-3-hydroxy-4H-benzo[e][1,2,4]thiadiazine 1,1-dioxide INT 17A (37 mg, 110 μmol, 23% yield). LCMS (m/z) calculated for C₁₄H₁₂ClN₃O₃S: 337.0; found 338.2 [M+H]⁺, t_{R}=3.88 min (Purity Method 4). ¹H NMR (400 MHz, DMSO) δ 11.08 (s, 1H), 10.45 (s, 1H), 7.99 (s, 1H), 7.39-7.32 (m, 2H), 7.30 (dd, J=8.0, 1.1 Hz, 1H), 7.24 (td, J=7.0, 6.4, 3.9 Hz, 2H), 7.15 (dd, J=7.8, 1.2 Hz, 1H), 7.07 (t, J=7.9 Hz, 1H), 4.11 (s, 2H).

### Step 17-2. Synthesis of 3-chloro-5-((2-chlorobenzyl)amino)-4H-benzo[e][1,2,4]thiadiazine 1,1-dioxide (INT 17B)

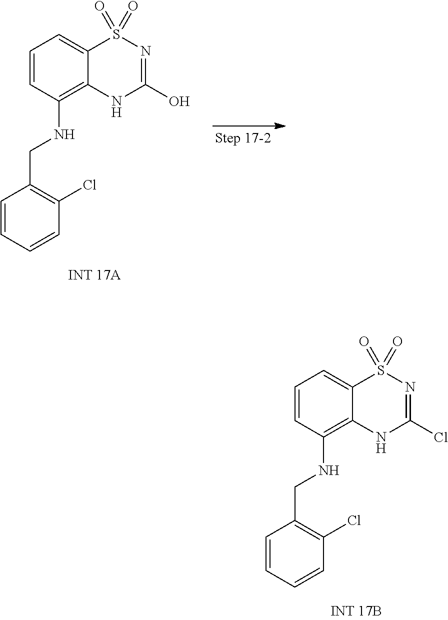 [SMILES: O=S1(=O)N=C(O)Nc2c(NCc3ccccc3Cl)cccc21 ; O=S1(=O)N=C(Cl)Nc2c(NCc3ccccc3Cl)cccc21]

To 5-((2-chlorobenzyl)amino)-3-hydroxy-4H-benzo[e][1,2,4]thiadiazine 1,1-dioxide INT 17A (37 mg, 1 Eq, 110 mol) was add 1 ml POCl₃. The mixture was stirred at 105° C. for 2h then concentrated in vacuo to obtain 3-chloro-5-((2-chlorobenzyl)amino)-4H-benzo[e][1,2,4]thiadiazine 1,1-dioxide INT 17B (35 mg, 93 mol, 20% yield) that was used without further purification. LCMS (m/z) calculated for C₁₄H₁₁Cl₂N₃O₂S: 355.0; found 356.0 [M+H]⁺, t_{R}=3.88 min (Purity Method 4).

### Step 17-3. Synthesis of 5-((2-chlorobenzyl)amino)-3-(((3-fluoropyridin-2-yl)methyl)amino)-4H-benzo[e][1,2,4]thiadiazine 1,1-dioxide (17-1)

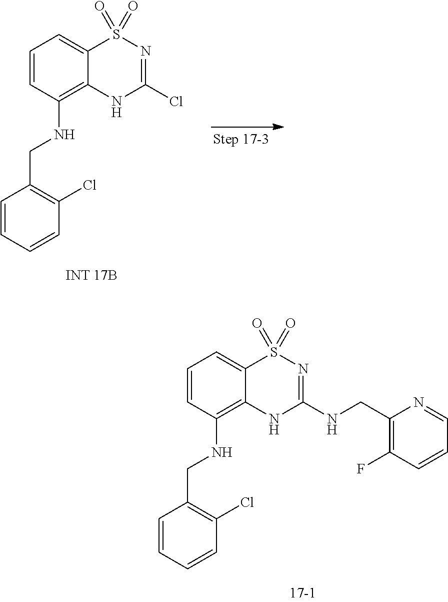 [SMILES: O=S1(=O)N=C(Cl)Nc2c(NCc3ccccc3Cl)cccc21 ; O=S1(=O)N=C(NCc2ncccc2F)Nc2c(NCc3ccccc3Cl)cccc21]

To a stirring solution of 3-chloro-5-((2-chlorobenzyl)amino)-4H-benzo[e][1,2,4]thiadiazine 1,1-dioxide INT 17B (8 mg, 1 Eq, 0.02 mmol) in NMP (2 mL) were added (3-fluoropyridin-2-yl)methanamine (4 mg, 1.3 Eq, 0.03 mmol) and cesium carbonate (20 mg, 3 Eq, 61 mol). The mixture was heated to 150° C. and stirred for 18 h. The reaction mixture was cooled to room temperature and water (4 mL) was added. The aqueous layer was extracted with EtOAc (3×5 mL). The combined organics were concentrated in vacuo and the crude residue was purified by silica gel chromatography (0-80% EtOAc/hexanes) to afford 5-((2-chlorobenzyl)amino)-3-(((3-fluoropyridin-2-yl)methyl)amino)-4H-benzo[e][1,2,4]thiadiazine 1,1-dioxide 17-1 (1.1 mg, 11% yield). LCMS (m/z) calculated for C₂₀H₁₇ClFN₅O₂S: 445.1; found 446.0 [M+H]⁺, t_{R}=5.798 min (Purity Method 3).

### Synthesis of Scheme 17 Compounds

The compounds listed in Table 18 were made using the procedures of Scheme 17.

TABLE 18

| | | Purity | Exact | | | |
|---|---|---|---|---|---|---|
| Cpd | | RT | mass | observed | | Purity |
| No. | Structure | (min) | (g/mol) | m/z | Ion | Method |
| 17-1 | 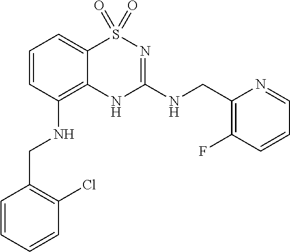 [SMILES: O=S1(=O)N=C(NCc2ncccc2F)Nc2c(NCc3ccccc3Cl)cccc21] | 5.798 | 445.1 | 446.0 | [M + H]⁺ | 3 |

### Example 18

### Synthesis of Representative Compound 18-1

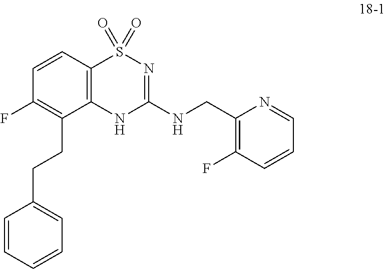 [SMILES: O=S1(=O)N=C(NCc2ncccc2F)Nc2c1ccc(F)c2CCc1ccccc1]

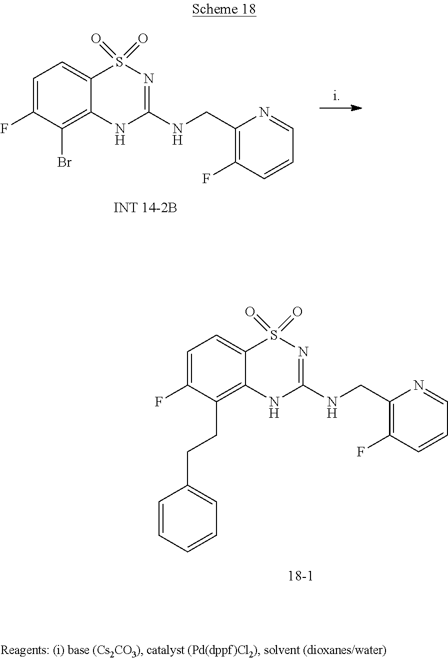 [SMILES: O=S1(=O)N=C(NCc2ncccc2F)Nc2c1ccc(F)c2Br ; O=S1(=O)N=C(NCc2ncccc2F)Nc2c1ccc(F)c2CCc1ccccc1]

### Step 18-1. Synthesis of 6-fluoro-3-(((3-fluoropyridin-2-yl)methyl)amino)-5-phenethyl-4H-benzo[e][1,2,4]thiadiazine 1,1-dioxide (18-1)

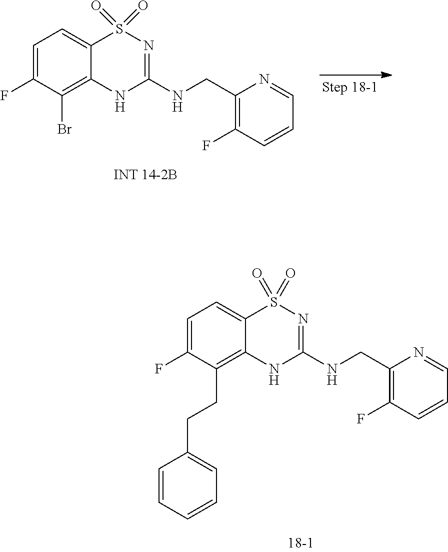 [SMILES: O=S1(=O)N=C(NCc2ncccc2F)Nc2c1ccc(F)c2Br ; O=S1(=O)N=C(NCc2ncccc2F)Nc2c1ccc(F)c2CCc1ccccc1]

To a solution of 5-bromo-6-fluoro-3-(((3-fluoropyridin-2-yl)methyl)amino)-4H-benzo[e][1,2,4]thiadiazine 1,1-dioxide INT 14-2B (15 mg, 1 Eq, 37 mol) and phenethylboronic acid (17 mg, 3 Eq, 0.11 mmol) in 1,4-Dioxane (2 mL) and Water (2 mL), Pd(dppf)C12 (2.7 mg, 0.1 Eq, 3.7 mol) and cesium carbonate (24 mg, 2 Eq, 74 mol) were added. The mixture was degasssed by N₂ and then the reaction was stirred at 90° C. for 3 h. The reaction mixture was passed through a syringe filter into a test tube, and the filtrate was directly loaded/purified by HPLC to afford 6-fluoro-3-(((3-fluoropyridin-2-yl)methyl)amino)-5-phenethyl-4H-benzo[e][1,2,4]-thiadiazine 1,1-dioxide 18-1 (0.7 mg, 2 mol, 4% yield). LCMS (m/z) calculated for C₂₁H₁₈F₂N₄O₂S: 428.1; found 429.1 [M+H]⁺, t_{R}=8.69 min (Purity Method 3).

The compounds listed in Table 19 were made using the procedures of Scheme 18.

TABLE 19

| | | Purity | Exact | | | |
|---|---|---|---|---|---|---|
| Cpd | | RT | mass | observed | | Purity |
| No. | Structure | (min) | (g/mol) | m/z | Ion | Method |
| 18-1 | 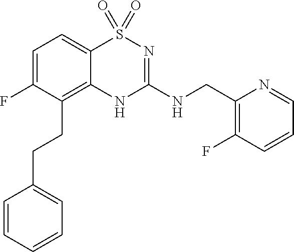 [SMILES: O=S1(=O)N=C(NCc2ncccc2F)Nc2c1ccc(F)c2CCc1ccccc1] | 8.69 | 428.1 | 429.1 | [M + H]⁺ | 3 |

### Example 19

### Synthesis of Representative Compound 19-1

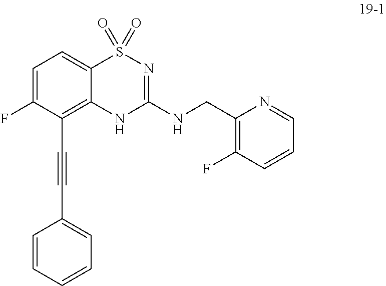 [SMILES: O=S1(=O)N=C(NCc2ncccc2F)Nc2c1ccc(F)c2C#Cc1ccccc1]

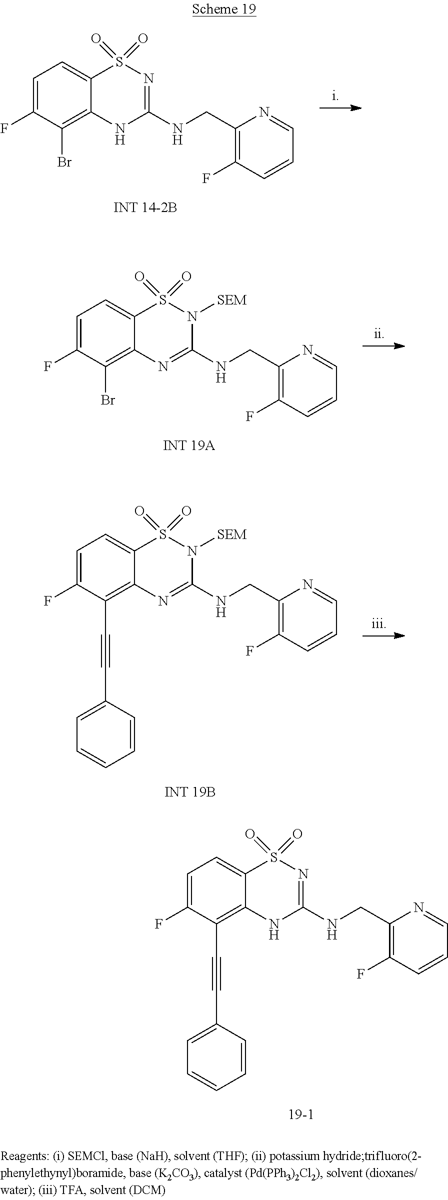 [SMILES: O=S1(=O)N=C(NCc2ncccc2F)Nc2c1ccc(F)c2Br ; C[Si](C)(C)CCOCN1C(NCc2ncccc2F)=Nc2c(ccc(F)c2Br)S1(=O)=O ; C[Si](C)(C)CCOCN1C(NCc2ncccc2F)=Nc2c(ccc(F)c2C#Cc2ccccc2)S1(=O)=O ; O=S1(=O)N=C(NCc2ncccc2F)Nc2c1ccc(F)c2C#Cc1ccccc1]

### Example Synthesis of Scheme 19 Compounds

### Step 19-1. Synthesis of 5-bromo-6-fluoro-3-(((3-fluoropyridin-2-yl)methyl)amino)-2-((2-(trimethylsilyl)ethoxy)methyl)-2H-benzo[e][1,2,4]thiadiazine 1,1-dioxide (INT 19A)

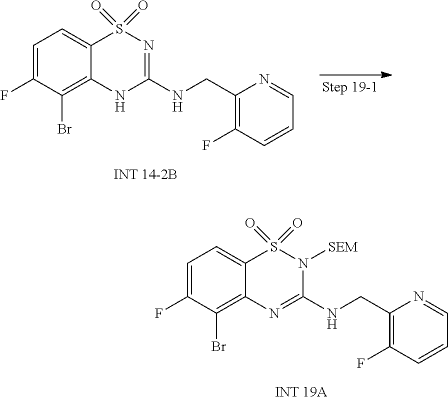 [SMILES: O=S1(=O)N=C(NCc2ncccc2F)Nc2c1ccc(F)c2Br ; C[Si](C)(C)CCOCN1C(NCc2ncccc2F)=Nc2c(ccc(F)c2Br)S1(=O)=O]

To a mixture of 5-bromo-6-fluoro-N-[(3-fluoro-2-pyridyl)methyl]-1,1-dioxo-2H-1,2,4-benzothiadiazin-3-amine INT 14-2B (1.8 g, 4.46 mmol, 1 eq) in THE (30 mL) was added NaH (540 mg, 13.50 mmol, 60% purity, 3.02 eq) at 0° C., and it was stirred at 0° C. for 0.5h. To the mixture was added SEM-CI (2.54 g, 15.26 mmol, 2.70 mL, 3.42 eq) at 0° C., and it was then stirred at 25° C. for 15 h. The mixture was quenched by adding 20 mL water, then extracted with ethyl acetate (3×20 mL). The combined organics were dried over anhydrous sodium sulfate, filtered, and concentrated in vacuo to give a crude residue. The residue was purified by flash silica gel chromatography (0-100% Ethyl acetate/petroleum ether gradient at 40 mL/min) to afford 5-bromo-6-fluoro-3-(((3-fluoropyridin-2-yl)methyl)amino)-2-((2-(trimethylsilyl)ethoxy)methyl)-2H-benzo[e][1,2,4]thiadiazine 1,1-dioxide INT 19A (250 mg, 337 mol, 8% yield) that was used without further purification. LCMS (m/z) calculated for C₁₉H₂₃BrF₂N₄O₈SSi: 532.0; found 533.2 [M+H]⁺, t_{R}=0.654 min (Purity Method 2). ¹H NMR (400 MHz, DMSO-d6) δ=13.03-12.16 (br, 1H), 8.60-8.35 (m, 1H), 8.02-7.74 (m, 2H), 7.69-7.49 (m, 1H), 7.43 (q, J=8.4 Hz, 1H), 5.28-5.02 (m, 2H), 4.97 (br d, J=7.1 Hz, 2H), 3.60-3.48 (m, 2H), 0.96-0.74 (m, 2H), 0.08 (m, 9H).

### Step 19-2. Synthesis of 6-fluoro-3-(((3-fluoropyridin-2-yl)methyl)amino)-5-(phenylethynyl)-2-((2-(trimethylsilyl)ethoxy)methyl)-2H-benzo[e][1,2,4]thiadiazine 1,1-dioxide (INT 19B)

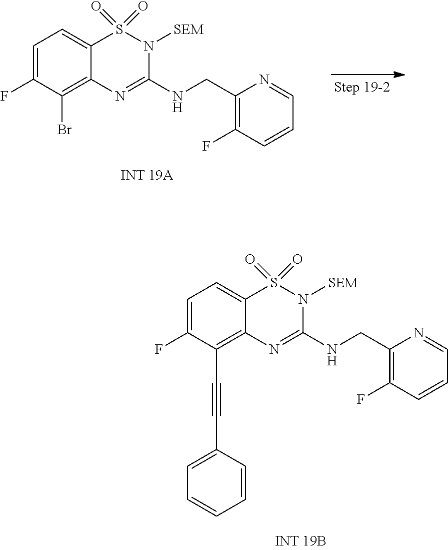 [SMILES: C[Si](C)(C)CCOCN1C(NCc2ncccc2F)=Nc2c(ccc(F)c2Br)S1(=O)=O ; C[Si](C)(C)CCOCN1C(NCc2ncccc2F)=Nc2c(ccc(F)c2C#Cc2ccccc2)S1(=O)=O]

To a mixture of 5-bromo-6-fluoro-N-[(3-fluoro-2-pyridyl)methyl]-1,1-dioxo-2-(2-trimethylsilylethoxymethyl)-1,2,4-benzothiadiazin-3-amine INT 19A (150 mg, 281 mol, 1 eq) and potassium trifluoro(2-phenylethynyl)boranuide (90.0 mg, 433 mol, 1.54 eq) in dioxane (10 mL) and Water (2 mL) were added K₂CO₃ (75.0 mg, 543 mol, 1.93 eq) and Pd(dppf)C12 (22.5 mg, 30.8 mol, 1.09e-1 eq). The reaction mixture was stirred at 60° C. for 15 h. The mixture was added to 10 mL water and extracted with ethyl acetate (3×15 mL). The combined organic layers were dried over anhydrous sodium sulfate, filtered, and concentrated in vacuo. The residue was purified by prep-TLC (PE/EA=1/1) to afford 6-fluoro-3-(((3-fluoropyridin-2-yl)methyl)amino)-5-(phenylethynyl)-2-((2-(trimethylsilyl)ethoxy)methyl)-2H-benzo[e][1,2,4]thiadiazine 1,1-dioxide INT 19B (40 mg, 85 mol, 30% yield). LCMS (m/z) calculated for C₂₇H₂₈F₂N₄O₃SSi: 554.2; found 555.2 [M+H]⁺, t_{R}=0.658 min (Purity Method 2).

### Step 19-3. Synthesis of 6-fluoro-3-(((3-fluoropyridin-2-yl)methyl)amino)-5-(phenylethynyl)-4H-benzo[e][1,2,4]thiadiazine 1,1-dioxide (19-1)

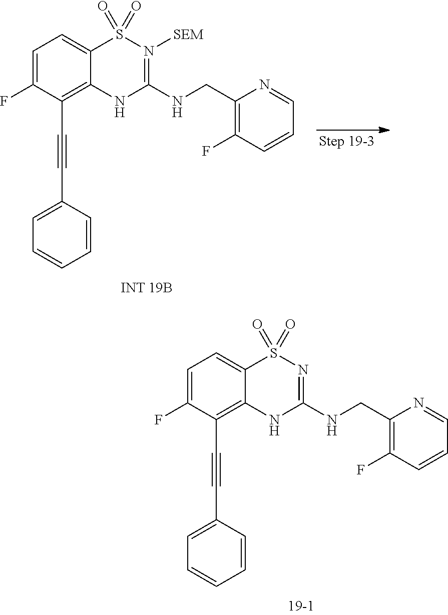

To a solution of 6-fluoro-N-[(3-fluoro-2-pyridyl)methyl]-1,1-dioxo-5-(2-phenylethynyl)-2-(2-trimethylsilylethoxymethyl)-1,2,4-benzothiadiazin-3-amine INT 19B (40 mg, 46.9 mol, 1 eq) in DCM (1 mL) was added TFA (1.54 g, 13.5 mmol, 1 mL, 287 eq). The reaction mixture was stirred at 25° C. for 4 h. The reaction mixture was concentrated under vacuum to give a crude residue. The residue was purified by prep-HPLC (column: Phenomenex Luna C18 150×25 mm×10 um; A-B mobile phase: [water(FA)-ACN]; gradient: 40%-70% B over 9 min) to afford 6-fluoro-3-(((3-fluoropyridin-2-yl)methyl)amino)-5-(phenylethynyl)-4H-benzo[e][1,2,4]-thiadiazine 1,1-dioxide 19-1 (0.6 mg, 1.41 mol, 3% yield). ¹H NMR (400 MHz, DMSO-d6) δ=10.33-10.11 (m, 1H), 8.65-8.50 (m, 1H), 8.45 (br d, J=3.9 Hz, 1H), 7.88-7.65 (m, 4H), 7.57-7.42 (m, 4H), 7.33-7.20 (m, 1H), 4.71 (br d, J=3.3 Hz, 2H). LCMS (m/z) calculated for C₂₁H₁₄F₂N₄O₂S: 424.1; found 425.1 [M+H]⁺, t_{R}=0.536 min (Purity Method 1).

The compounds listed in Table 20 were made using the procedures of Scheme 19.

TABLE 20

| | | Purity | Exact | | | |
|---|---|---|---|---|---|---|
| Cpd | | RT | mass | observed | | Purity |
| No. | Structure | (min) | (g/mol) | m/z | Ion | Method |
| 19-1 | 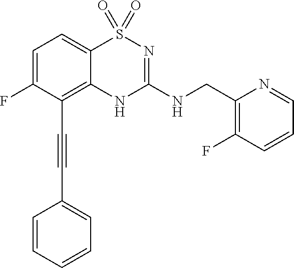 [SMILES: O=S1(=O)N=C(NCc2ncccc2F)Nc2c1ccc(F)c2C#Cc1ccccc1] | 0.536 | 424.1 | 425.1 | [M + H]⁺ | 1 |

### Example 20

### Synthesis of Representative Compound 20-1

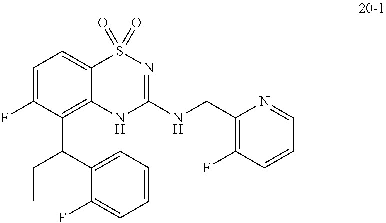 [SMILES: CCC(c1ccccc1F)c1c(F)ccc2c1NC(NCc1ncccc1F)=NS2(=O)=O]

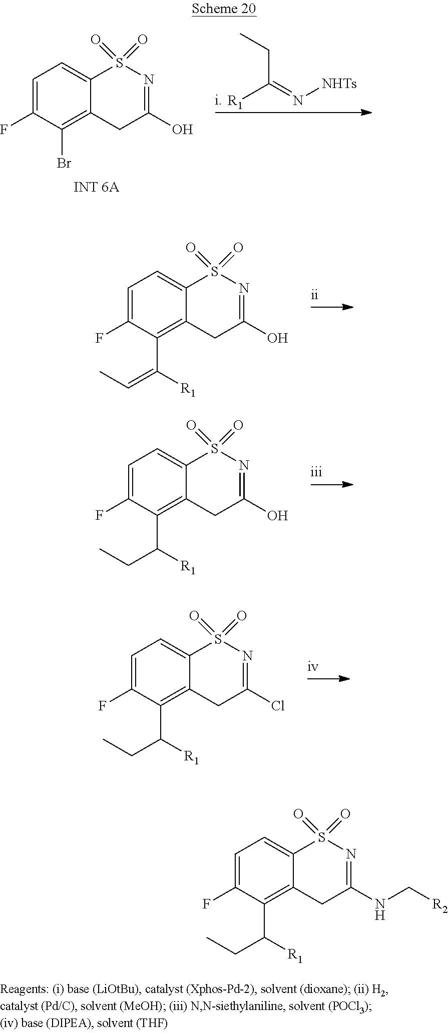

### Example Synthesis of Scheme 20 Compounds

### Step 20-1. Synthesis of (E)-N′-(1-(2-fluorophenyl)propylidene)-4-methylbenzenesulfonohydrazide (INT 20A)

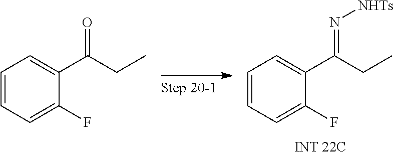

A mixture of 1-(2-fluorophenyl)propan-1-one (2 g, 13.14 mmol, 1 eq) and 4-methylbenzenesulfonohydrazide (2.45 g, 13.14 mmol, 1 eq) in MeOH (40 mL) was stirred at 60° C. for 15 h. The mixture was concentrated in vacuo to afford (E)-N′-(1-(2-fluorophenyl)propylidene)-4-methylbenzenesulfonohydrazide INT 20A (4.4 g, 11.7 mmol, 89% yield) that was used without further purification. ¹H NMR (400 MHz, CHLOROFORM-d) δ=7.92-7.80 (m, 2H), 7.49-7.37 (m, 1H), 7.35 (d, J=7.9 Hz, 2H), 7.28-7.22 (m, 1H), 7.20-7.11 (m, 1H), 7.09-7.01 (m, 1H), 2.62-2.41 (m, 5H), 1.13-1.00 (m, 3H).

### Step 20-2. Synthesis of 6-fluoro-5-(1-(2-fluorophenyl)prop-1-en-1-yl)-3-hydroxy-4H-benzo[e][1,2,4]thiadiazine 1,1-dioxide (INT 20B)

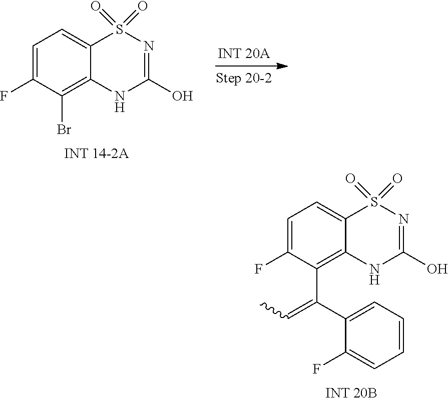

To a solution of N—[(Z)-1-(2-fluorophenyl)propylideneamino]-4-methyl-benzenesulfonamide INT 20B (1.63 g, 5.08 mmol, 1.5 eq) and 5-bromo-6-fluoro-1,1-dioxo-4H-1,2,4-benzothiadiazin-3-ol INT 14-2A (1.0 g, 3.39 mmol, 1 eq) in dioxane (20 mL) were added lithium tert-butoxide (543 mg, 6.78 mmol, 611 μL, 2 eq) and Xphos-Pd-G2 (267 mg, 339 mol, 0.1 eq). The reaction mixture was stirred at 100° C. for 15 h. The reaction mixture was concentrated in vacuo to give a residue. To the residue was added ammonium chloride solution (30 mL). The aqueous layer was extracted with ethyl acetate (2×30 mL). The combined organics were washed with brine (2×20 mL), dried over anhydrous sodium sulfate, filtered, and concentrated in vacuo to give a residue. The residue was purified by prep-HPLC (column: Waters Xbridge Prep OBD C18 150×40 mm×10 um; A-B mobile phase: [water(NH₄HCO₃)-ACN]; gradient: 12%-42% B over min) to afford 6-fluoro-5-(1-(2-fluorophenyl)prop-1-en-1-yl)-3-hydroxy-4H-benzo[e][1,2,4]thiadiazine 1,1-dioxide INT 20B (150 mg, 409.19 mol, 12% yield).

### Step 20-3. Synthesis of 6-fluoro-5-(1-(2-fluorophenyl)propyl)-3-hydroxy-4H-benzo[e][1,2,4]thiadiazine 1,1-dioxide (INT 20C)

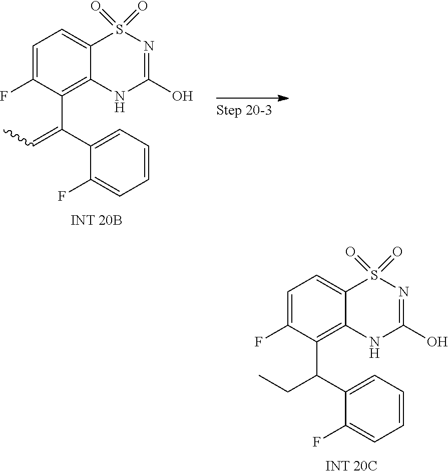

A mixture of 6-fluoro-5-(1-(2-fluorophenyl)prop-1-en-1-yl)-3-hydroxy-4H-benzo[e][1,2,4]thiadiazine 1,1-dioxide INT 20B (150 mg, 428 mol, 1 eq) and Pd/C (100 mg, 10% purity) in MeOH (10 mL) was degassed and purged with N₂ 3 times, and then the mixture was stirred at 50° C. for 15 h under an H2 atmosphere (50 psi). The reaction mixture was filtered and concentrated in vacuo to afford 6-fluoro-5-(1-(2-fluorophenyl)propyl)-3-hydroxy-4H-benzo[e][1,2,4]thiadiazine 1,1-dioxide INT 20C (130 mg, 328 mol, 77% yield) that was used without further purification.

### Step 20-4. Synthesis of 3-chloro-6-fluoro-5-(1-(2-fluorophenyl)propyl)-4H-benzo[e][1,2,4]thiadiazine 1,1-dioxide (INT 20D)

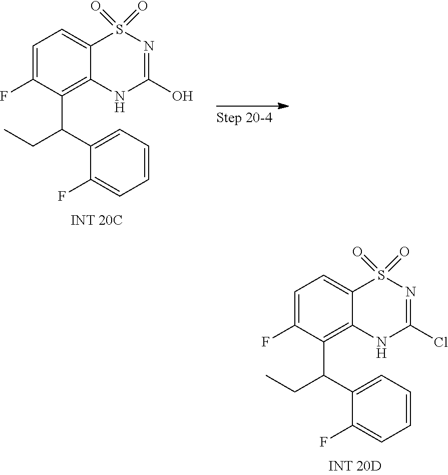 [SMILES: CCC(c1ccccc1F)c1c(F)ccc2c1NC(O)=NS2(=O)=O ; CCC(c1ccccc1F)c1c(F)ccc2c1NC(Cl)=NS2(=O)=O]

To a solution of 6-fluoro-5-(1-(2-fluorophenyl)propyl)-3-hydroxy-4H-benzo[e][1,2,4]thiadiazine 1,1-dioxide INT 20C (130 mg, 369 mol, 1 eq) in POCl₃ (2 mL) was added N,N-diethylaniline (82.6 mg, 553 mol, 89 μL, 1.5 eq). The mixture was stirred at 120° C. for 6 h. The reaction was quenched by addition of sodium bicarbonate solution (20 mL). The aqueous layer was extracted with ethyl acetate (3×15 mL), washed with brine (2×10 mL), filtered, and concentrated in vacuo to give a residue. The residue was purified by flash silica gel chromatography (0-100% Ethyl acetate/petroleum ether gradient at 20 mL/min) to afford 3-chloro-6-fluoro-5-(1-(2-fluorophenyl)propyl)-4H-benzo[e][1,2,4]thiadiazine 1,1-dioxide INT 20D (60 mg, 148 mol, 40% yield).

### Step 20-5. Synthesis of 6-fluoro-5-(1-(2-fluorophenyl)propyl)-3-(((3-fluoropyridin-2-yl)methyl)amino)-4H-benzo[e][1,2,4]thiadiazine 1,1-dioxide (20-1)

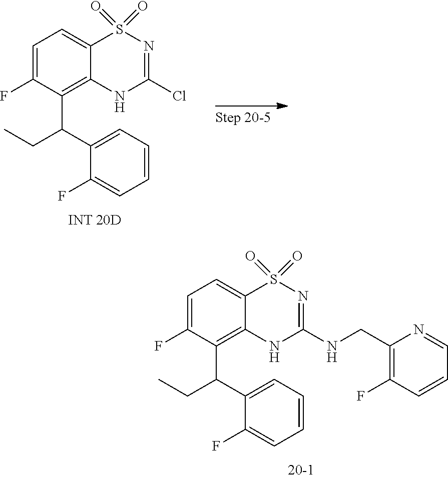 [SMILES: CCC(c1ccccc1F)c1c(F)ccc2c1NC(Cl)=NS2(=O)=O ; CCC(c1ccccc1F)c1c(F)ccc2c1NC(NCc1ncccc1F)=NS2(=O)=O]

To a solution of 3-chloro-6-fluoro-5-(1-(2-fluorophenyl)propyl)-4H-benzo[e][1,2,4]thiadiazine 1,1-dioxide INT 20D (60 mg, 162 mol, 1 eq) and (3-fluoro-2-pyridyl)methanamine;dihydrochloride (64.4 mg, 324 mol, 2 eq) in THE (2 mL) was added DIPEA (105 mg, 809 mol, 141 μL, 5 eq). The mixture was stirred at 60° C. for 15 h. The reaction mixture was concentrated under reduced pressure to give a residue. The residue was purified by prep-HPLC (column: C18 150×30 mm; A-B mobile phase: [water(FA)-ACN]; gradient: 55%-65% B over 7 min) to afford 6-fluoro-5-(1-(2-fluorophenyl)propyl)-3-(((3-fluoropyridin-2-yl)methyl)amino)-4H-benzo[e][1,2,4]thiadiazine 1,1-dioxide 20-1 (10.1 mg, 21.9 mol, 14% yield). LCMS (m/z) calculated for C₂₂H₁₉F₃N₄O₂S: 460.1; found 461.1 [M+H]⁺, t_{R}=0.527 min (Purity Method 1). ¹H NMR (400 MHz, DMSO-d6) δ=10.59-10.40 (m, 1H), 8.48 (br d, J=4.5 Hz, 1H), 8.18 (br s, 1H), 7.80 (t, J=8.8 Hz, 1H), 7.66 (dd, J=5.6, 8.7 Hz, 1H), 7.59 (br t, J=7.8 Hz, 1H), 7.50 (td, J=4.5, 8.5 Hz, 1H), 7.35-7.21 (m, 2H), 7.15-7.07 (m, 1H), 7.01 (dd, J=8.9, 11.1 Hz, 1H), 4.79-4.64 (m, 2H), 4.52 (br t, J=7.3 Hz, 1H), 2.30-2.12 (m, 2H), 2.06 (s, 1H), 0.95-0.87 (m, 3H).

The compounds listed in Table 21 were made using the procedures of Scheme 20.

TABLE 21

| | | Purity | Exact | | | |
|---|---|---|---|---|---|---|
| Cpd | | RT | mass | observed | | Purity |
| No. | Structure | (min) | (g/mol) | m/z | Ion | Method |
| 20-1 | 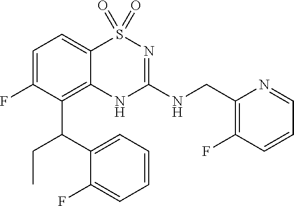 [SMILES: CCC(c1ccccc1F)c1c(F)ccc2c1NC(NCc1ncccc1F)=NS2(=O)=O] | 0.527 | 460.1 | 461.1 | [M + H]⁺ | 1 |

### Example 21

### Synthesis of Representative Compound 21-1

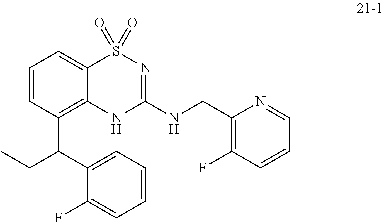 [SMILES: CCC(c1ccccc1F)c1cccc2c1NC(NCc1ncccc1F)=NS2(=O)=O]

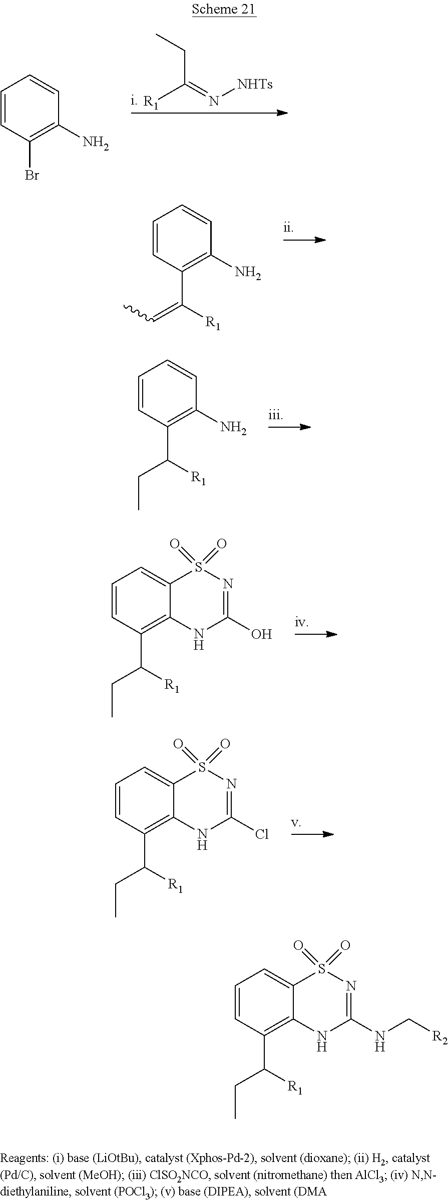

### Example Synthesis of Scheme 21 Compounds

### Step 21-1. Synthesis of 2-(1-(2-fluorophenyl)prop-1-en-1-yl)aniline (INT 21A)

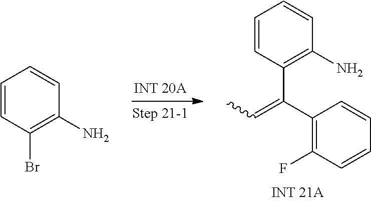

To a solution of N-[(E)-1-(2-fluorophenyl)propylideneamino]-4-methyl-benzenesulfonamide INT 20A (2.0 g, 5.31 mmol, 9.13e-1 eq) and 2-bromoaniline (1 g, 5.81 mmol, 1 eq) in dioxane (20 mL) were added t-BuOLi (931 mg, 11.63 mmol, 1.05 mL, 2 eq) and XPhos Pd G3 (492 mg, 581 mol, 0.1 eq). The mixture was stirred at 100° C. for 14 h. The reaction mixture was poured into water (40 mL). The aqueous layer was extracted with EtOAc (2×30 mL). The combined organics were washed by brine (3×30 mL), dried over Na₂SO₄, filtered, and concentrated in vacuo to give a residue. The residue was purified by flash silica gel chromatography (0-10% Ethyl acetate/Petroleum ether gradient) to afford 2-(1-(2-fluorophenyl)prop-1-en-1-yl)aniline INT 21A (0.8 g, 3.27 mmol, 56% yield). LCMS (m/z) calculated for C₁₅H₁₄FN: 227.1; found 228.1 [M+H]⁺, t_{R}=0.527 min (Purity Method 1).

### Step 21-2. Synthesis of 2-(1-(2-fluorophenyl)propyl)aniline (INT 21B)

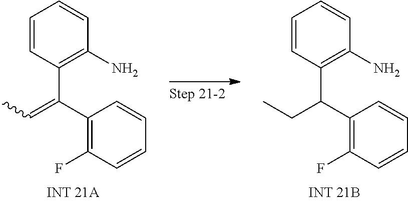

To a solution of 2-[(Z)-1-(2-fluorophenyl)prop-1-enyl]aniline INT 21A (0.8 g, 3.52 mmol, 1 eq) in MeOH (25 mL) was added 10% Pd/C (108 mg, 101 mol, 0.03 eq) under N₂ atmosphere. The suspension was degassed and purged with H2 3 times. The mixture was stirred under an atmosphere of H2 (50 Psi) at 50° C. for 14 h. The crude mixture was filtered through a pad of celite and the filtrate was concentrated in vacuo to afford 2-(1-(2-fluorophenyl)propyl)aniline INT 21B (0.8 g, crude) that was carried on without further purification. LCMS (m/z) calculated for C₁₅H₁₆FN: 229.1; found 230.0 [M+H]⁺, t_{R}=0.528 min (Purity Method 1).

### Step 21-3. Synthesis of 5-(1-(2-fluorophenyl)propyl)-3-hydroxy-4H-benzo[e][1,2,4]thiadiazine 1,1-dioxide (INT 21C)

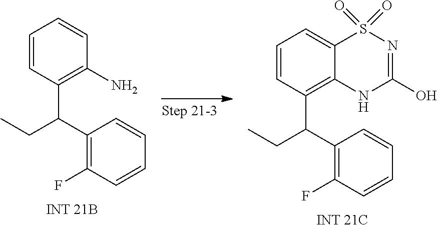 [SMILES: CCC(c1ccccc1N)c1ccccc1F ; CCC(c1ccccc1F)c1cccc2c1NC(O)=NS2(=O)=O]

To a solution of 2-[1-(2-fluorophenyl)propyl]aniline INT 21B (0.8 g, 3.49 mmol, 1 eq) in nitrobenzene (12 mL) was added N-(oxomethylene)sulfamoyl chloride (741 mg, 5.23 mmol, 454 μL, 1.5 eq) at 0° C. The mixture was stirred at 0° C. for 0.5 h. Then, AlCl₃ (582 mg, 4.36 mmol, 238 μL, 1.25 eq) was added. The mixture was stirred at 80° C. for 1.5 h. The reaction solution was cooled to room temperature and then diluted with saturated NH₄Cl aqueous solution (100 mL). The aqueous phase was extracted with ethyl acetate (3×80 mL). The combined organic layers were washed with brine (50 mL), dried over anhydrous sodium sulfate, filtered, and concentrated under vacuum to give a brown oil. The obtained brown oil was diluted with petroleum ether (150 mL) and then stirred for 30 min. The brown oil eventually formed a brown precipitate that was collected by vacuum filtration. The filter cake was washed with petroleum ether (60 mL) and then dried under vacuum to give a brown solid. The precipitate was triturated in toluene (3 mL) and collected by filtration to afford 5-(1-(2-fluorophenyl)propyl)-3-hydroxy-4H-benzo[e][1,2,4]thiadiazine 1,1-dioxide INT 21C (0.5 g, 1.44 mmol, 41% yield). LCMS (m/z) calculated for C₁₆H₁₅FN₂O₃S: 334.1; found 333.1 [M−H], t_{R}=0.453 min (Purity Method 2 negative mode).

### Step 21-4. Synthesis of 3-chloro-5-(1-(2-fluorophenyl)propyl)-4H-benzo[e][1,2,4]thiadiazine 1,1-dioxide (INT 21D)

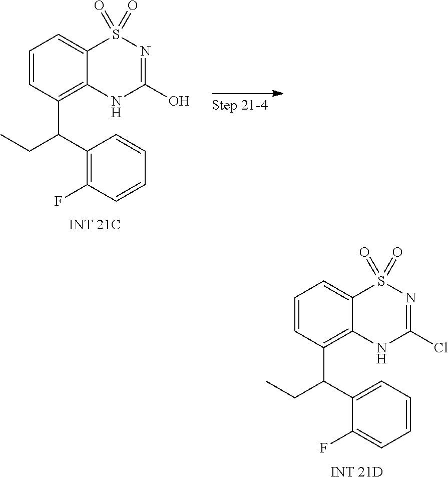 [SMILES: CCC(c1ccccc1F)c1cccc2c1NC(O)=NS2(=O)=O ; CCC(c1ccccc1F)c1cccc2c1NC(Cl)=NS2(=O)=O]

To a solution of POCl₃ (4.59 g, 29.9 mmol, 2.79 mL, 20 eq) in toluene (7.5 mL), were added 5-[1-(2-fluorophenyl)propyl]-1,1-dioxo-4H-1,2,4-benzothiadiazin-3-ol INT 21C (0.5 g, 1.50 mmol, 1 eq) and N,N-diethylaniline (335 mg, 2.24 mmol, 359 μL, 1.5 eq). The mixture was stirred at 110° C. for 16 h. The reaction solution was poured into water (50 mL) portionwise while the temperature was kept below 35° C. The obtained mixture was stirred for 1 h. The mixture was extracted with EtOAc (3×50 mL). The combined organics were washed with brine (2×50 mL), dried over Na₂SO₄, filtered, and concentrated in vacuo to give a residue. The residue was purified by flash silica gel chromatography (0-50% Ethyl acetate/petroleum ether gradient at 50 mL/min) to afford 3-chloro-5-(1-(2-fluorophenyl)propyl)-4H-benzo[e][1,2,4]thiadiazine 1,1-dioxide INT 21De (0.4 g, 1.05 mmol, 71% yield). LCMS (m/z) calculated for C₁₆H₁₄ClFN₂O₂S: 352.0; found 351.0 [M−H], t_{R}=0.520 min (Purity Method 2 negative mode).

### Step 21-5. Synthesis of 5-(1-(2-fluorophenyl)propyl)-3-(((3-fluoropyridin-2-yl)methyl)amino)-4H-benzo[e][1,2,4]thiadiazine 1,1-dioxide (21-1)

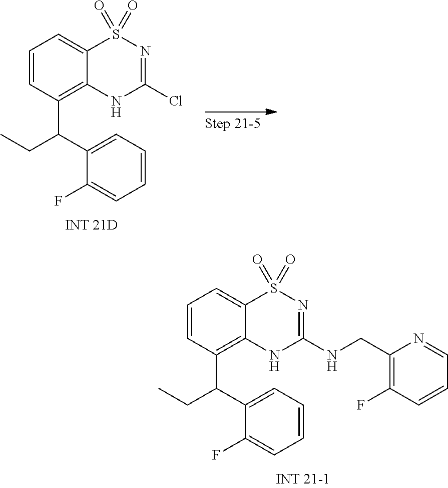 [SMILES: CCC(c1ccccc1F)c1cccc2c1NC(Cl)=NS2(=O)=O ; CCC(c1ccccc1F)c1cccc2c1NC(NCc1ncccc1F)=NS2(=O)=O]

To a solution of 3-chloro-5-[1-(2-fluorophenyl)propyl]-4H-1,2,4-benzothiadiazine 1,1-dioxide INT 21D (150 mg, 425 mol, 1 eq), (3-fluoro-2-pyridyl)methanamine.dihydrochloride (102 mg, 510 mol, 1.2 eq) in DMA (3 mL) was added DIPEA (281 mg, 2.18 mmol, 379 μL, 5.12 eq). The mixture was stirred at 120° C. for 14 h. The crude mixture was filtered. The residue was purified by preparative HPLC (column: Welch Xtimate C18 150×25 mm×5 um; A-B mobile phase: [water(FA)-ACN]; gradient: 35%-65% B over 40 min) and lyophilized to afford 5-(1-(2-fluorophenyl)propyl)-3-(((3-fluoropyridin-2-yl)methyl)amino)-4H-benzo[e][1,2,4]thiadiazine 1,1-dioxide 21-1 (80 mg, 174 mol, 41% yield). LCMS (m/z) calculated for C₂₂H₂₀F₂N₄O₂S: 442.1; found 443.2 [M+H]⁺, t_{R}=0.56 min (Purity Method 1). ¹H NMR (400 MHz, DMSO-d6) δ=10.24 (br s, 1H), 8.49 (d, J=4.6 Hz, 1H), 8.19-8.10 (m, 1H), 7.85-7.77 (m, 1H), 7.60 (br t, J=7.1 Hz, 1H), 7.56 (dd, J=1.1, 7.6 Hz, 1H), 7.51 (td, J=4.4, 8.5 Hz, 1H), 7.37-7.24 (m, 3H), 7.23-7.08 (m, 2H), 4.84-4.63 (m, 2H), 4.55 (br t, J=7.1 Hz, 1H), 2.14 (td, J=7.2, 14.0 Hz, 1H), 2.07-1.92 (m, 1H), 0.90 (t, J=7.2 Hz, 3H).

The compounds listed in Table 22 were made using the procedures of Scheme 21.

TABLE 22

| | | Purity | Exact | | | |
|---|---|---|---|---|---|---|
| Cpd | | RT | mass | observed | | Purity |
| No. | Structure | (min) | (g/mol) | m/z | Ion | Method |
| 21-1 | 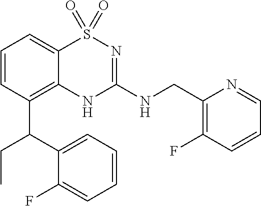 [SMILES: CCC(c1ccccc1F)c1cccc2c1NC(NCc1ncccc1F)=NS2(=O)=O] | 0.56 | 442.1 | 443.1 | [M + H]⁺ | 1 |

### Example 22

### Synthesis of Representative Compound 22-1

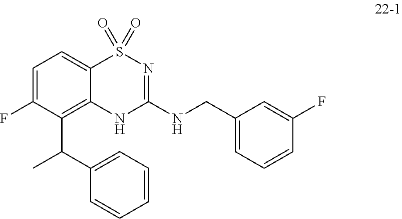 [SMILES: CC(c1ccccc1)c1c(F)ccc2c1NC(NCc1cccc(F)c1)=NS2(=O)=O]

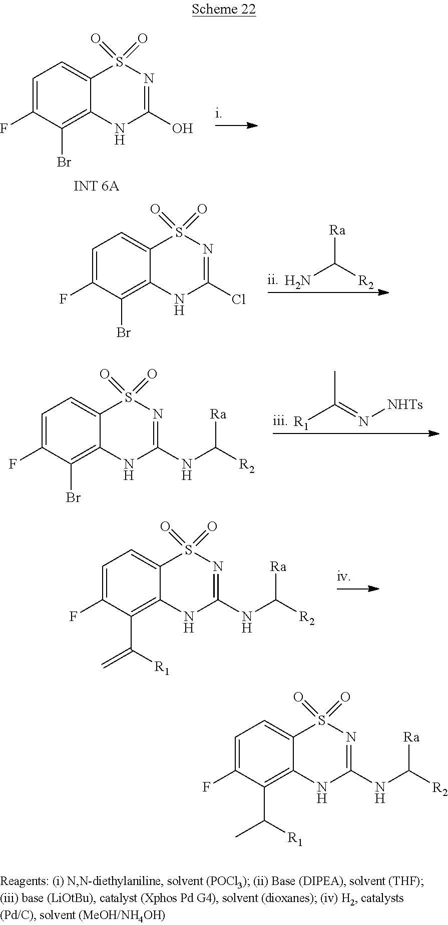

### Example Synthesis of Scheme 22 Compounds

### Step 22-1. Synthesis of 5-bromo-6-fluoro-3-((3-fluorobenzyl)amino)-4H-benzo[e][1,2,4]thiadiazine 1,1-dioxide (INT 22B)

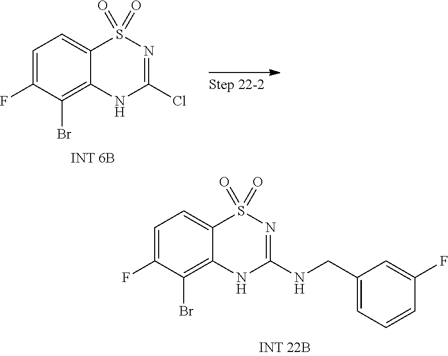 [SMILES: O=S1(=O)N=C(Cl)Nc2c1ccc(F)c2Br ; O=S1(=O)N=C(NCc2cccc(F)c2)Nc2c1ccc(F)c2Br]

To a solution of 5-bromo-3-chloro-6-fluoro-4H-benzo[e][1,2,4]thiadiazine 1,1-dioxide INT 6B (4.8 g, 15.3 mmol, 1 eq) and (3-fluorophenyl)methanamine (2.87 g, 23.0 mmol, 2.62 mL, 1.5 eq) in THE (50 mL) was added DIPEA (5.94 g, 45.9 mmol, 8.00 mL, 3 eq). The mixture was stirred at 60° C. for 15 h. The reaction mixture was filtered and concentrated in vacuo to afford 5-bromo-6-fluoro-3-((3-fluorobenzyl)amino)-4H-benzo[e][1,2,4]thiadiazine 1,1-dioxide INT 22B (5.3 g, 13.1 mmol, 86% yield, 99.6% purity). LCMS (m/z) calculated for C₁₄H₁₀BrF₂N₃O₂S: 401.0; found 401.9 [M+H]⁺, t_{R}=0.48 min (Purity Method 1).

### Step 22-2B. Synthesis of (E)-4-methyl-N′-(1-phenylethylidene)benzenesulfonohydrazide (INT 22C)

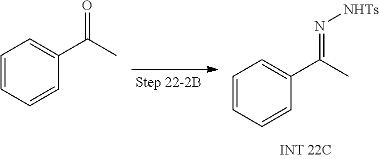

To a solution of 1-phenylethanone (1.0 g, 8.32 mmol, 973 μL, 1 eq) in MeOH (5 mL), was added 4-methylbenzenesulfonohydrazide (1.57 g, 8.41 mmol, 1.01 eq). The mixture was stirred at 60° C. for 1 h. A white precipitate was formed. The crude mixture was filtered and the obtained solid was dried under vacuum to afford (E)-4-methyl-N′-(1-phenylethylidene)benzenesulfonohydrazide INT 22C (1.5 g, 5.20 mmol, 63% yield). LCMS (m/z) calculated for C₁₅H₁₆N₂O₂S: 288.1; found 289.1 [M+H]⁺, t_{R}=0.528 min (Purity Method 1).

### Step 22-3. Synthesis of 6-fluoro-3-((3-fluorobenzyl)amino)-5-(1-phenylvinyl)-4H-benzo[e][1,2,4]thiadiazine 1,1-dioxide (INT 22D)

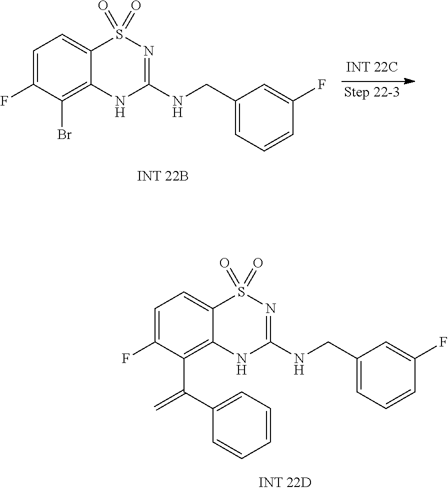 [SMILES: O=S1(=O)N=C(NCc2cccc(F)c2)Nc2c1ccc(F)c2Br ; C=C(c1ccccc1)c1c(F)ccc2c1NC(NCc1cccc(F)c1)=NS2(=O)=O]

To a solution of (E)-4-methyl-N′-(1-phenylethylidene)benzenesulfonohydrazide INT 22C (72 mg, 249 mol, 1 eq) and 5-bromo-6-fluoro-3-((3-fluorobenzyl)amino)-4H-benzo[e][1,2,4]thiadiazine 1,1-dioxide INT 22B (100 mg, 249 mol, 1 eq) in dioxane (2 mL) were added lithium tert-butoxide (40 mg, 497 mol, 45 μL, 2 eq) and Xphos Pd G4 (21 mg, 24.9 mol, 0.1 eq). The mixture was stirred at 100° C. for 15 h. The reaction mixture was concentrated in vacuo. To the residue was added saturated ammonium chloride solution (20 mL). The aqueous layer was extracted with ethyl acetate (2×20 mL). The combined organic layers were washed with brine (2×10 mL), dried over anhydrous sodium sulfate, filtered, and concentrated in vacuo to give 6-fluoro-3-((3-fluorobenzyl)amino)-5-(1-phenylvinyl)-4H-benzo[e][1,2,4]thiadiazine 1,1-dioxide INT 22D (110 mg) that was used without further purification. LCMS (m/z) calculated for C₂₂H₁₇F₂N₃O₂S: 425.1; found 426.1 [M+H]⁺, t_{R}=0.533 min (Purity Method 1).

### Step 22-4. Synthesis of 6-fluoro-3-((3-fluorobenzyl)amino)-5-(1-phenylethyl)-4H-benzo[e][1,2,4]thiadiazine 1,1-dioxide (22-1)

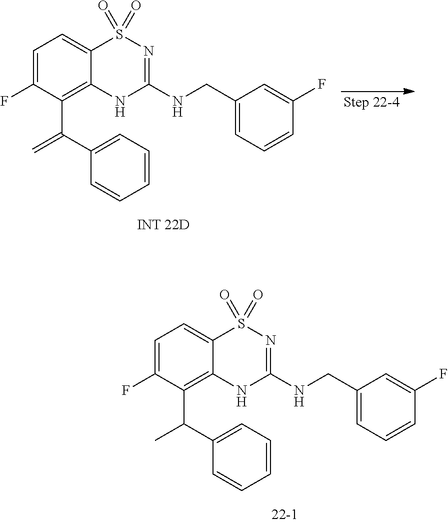 [SMILES: C=C(c1ccccc1)c1c(F)ccc2c1NC(NCc1cccc(F)c1)=NS2(=O)=O ; CC(c1ccccc1)c1c(F)ccc2c1NC(NCc1cccc(F)c1)=NS2(=O)=O]

A mixture of 6-fluoro-3-((3-fluorobenzyl)amino)-5-(1-phenylvinyl)-4H-benzo[e][1,2,4]thiadiazine 1,1-dioxide INT 22D (50 mg, 118 mol, 1 eq), NH₃·H₂O (142 mg, 1.18 mmol, 156 μL, 29% purity, 10 eq) and 10% Pd/C (50 mg) in MeOH (10 mL) was degassed and purged with N₂ 3 times. The reaction mixture was then heated at 50° C. for 15 h under an H₂ atmosphere (50 psi). The reaction mixture was filtered and concentrated in vacuo to give a crude residue. The residue was purified by prep-HPLC (column: Welch Xtimate C18 150×25 mm×5 um; mobile phase: [water(FA)-ACN];gradient:40%-70% B over 40 min) to afford 6-fluoro-3-((3-fluorobenzyl)amino)-5-(1-phenylethyl)-4H-benzo[e][1,2,4]thiadiazine 1,1-dioxide 22-1 (11.8 mg, 27.3 mol, 23% yield). LCMS (m/z) calculated for C₂₂H₁₉F₂N₃O₂S: 427.1; found 428.2 [M+H]⁺, t_{R}=0.533 min (Purity Method 1). ¹H NMR (400 MHz, DMSO-d6) δ=10.21 (s, 1H), 7.77 (br s, 1H), 7.67 (dd, J=5.6, 8.7 Hz, 1H), 7.47-7.39 (m, 1H), 7.35-7.29 (m, 2H), 7.27-7.19 (m, 5H), 7.16-7.09 (m, 1H), 7.05 (dd, J=8.8, 11.1 Hz, 1H), 4.62-4.44 (m, 3H), 1.70 (br d, J=6.8 Hz, 3H).

The compounds listed in Table 23 were made using the procedures of Scheme 22.

TABLE 23

| | | Purity | Exact | | | |
|---|---|---|---|---|---|---|
| Cpd | | RT | mass | observed | | Purity |
| No. | Structure | (min) | (g/mol) | m/z | Ion | Method |
| 22-1 | 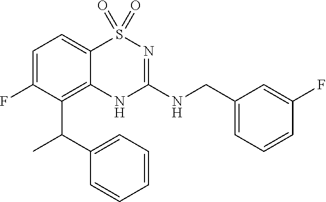 [SMILES: CC(c1ccccc1)c1c(F)ccc2c1NC(NCc1cccc(F)c1)=NS2(=O)=O] | 0.541 | 427.1 | 428.1 | [M + H]⁺ | 1 |
| 22-2 | 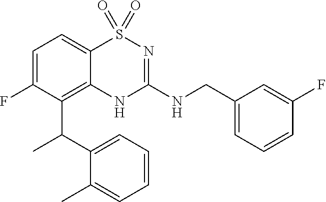 [SMILES: Cc1ccccc1C(C)c1c(F)ccc2c1NC(NCc1cccc(F)c1)=NS2(=O)=O] | 0.549 | 441.1 | 442.1 | [M + H]⁺ | 1 |
| 22-3 | 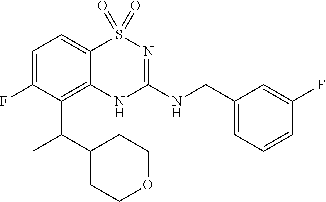 [SMILES: CC(c1c(F)ccc2c1NC(NCc1cccc(F)c1)=NS2(=O)=O)C1CCOCC1] | 0.460 | 435.1 | 436.2 | [M + H]⁺ | 2 |
| 22-4 | 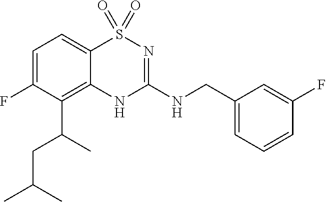 [SMILES: CC(C)CC(C)c1c(F)ccc2c1NC(NCc1cccc(F)c1)=NS2(=O)=O] | 0.602 | 407.1 | 408.2 | [M + H]⁺ | 1 |
| 22-5 | 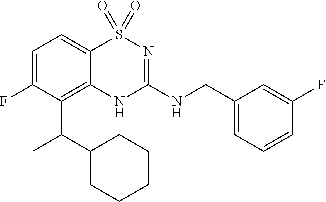 [SMILES: CC(c1c(F)ccc2c1NC(NCc1cccc(F)c1)=NS2(=O)=O)C1CCCCC1] | 0.587 | 433.2 | 434.2 | [M + H]⁺ | 2 |
| 22-6 | 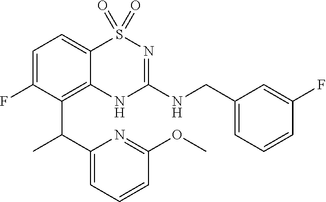 [SMILES: COc1cccc(C(C)c2c(F)ccc3c2NC(NCc2cccc(F)c2)=NS3(=O)=O)n1] | 0.524 | 458.1 | 459.1 | [M + H]⁺ | 1 |
| 22-7 | 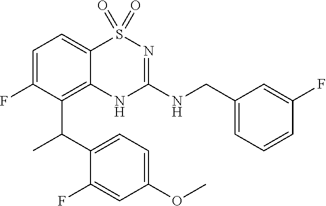 [SMILES: COc1ccc(C(C)c2c(F)ccc3c2NC(NCc2cccc(F)c2)=NS3(=O)=O)c(F)c1] | 0.525 | 475.1 | 476.0 | [M + H]⁺ | 1 |
| 22-8 | 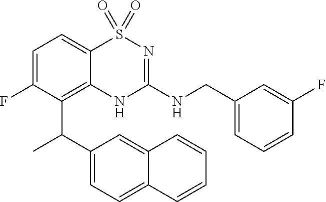 [SMILES: CC(c1ccc2ccccc2c1)c1c(F)ccc2c1NC(NCc1cccc(F)c1)=NS2(=O)=O] | 0.571 | 477.1 | 478.2 | [M + H]⁺ | 1 |
| 22-9 | 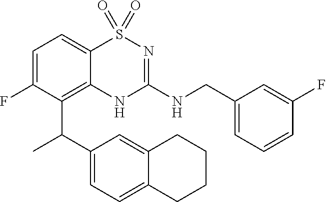 [SMILES: CC(c1ccc2c(c1)CCCC2)c1c(F)ccc2c1NC(NCc1cccc(F)c1)=NS2(=O)=O] | 0.598 | 481.2 | 482.1 | [M + H]⁺ | 1 |
| 22-10 | 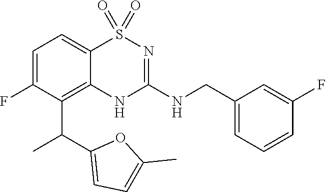 [SMILES: Cc1ccc(C(C)c2c(F)ccc3c2NC(NCc2cccc(F)c2)=NS3(=O)=O)o1] | 0.537 | 431.1 | 432.1 | [M + H]⁺ | 1 |
| 22-11 | 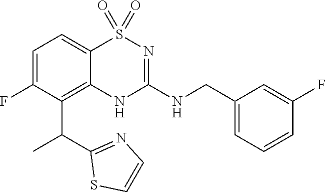 [SMILES: CC(c1nccs1)c1c(F)ccc2c1NC(NCc1cccc(F)c1)=NS2(=O)=O] | 0.504 | 434.1 | 435.0 | [M + H]⁺ | 1 |
| 22-12 | 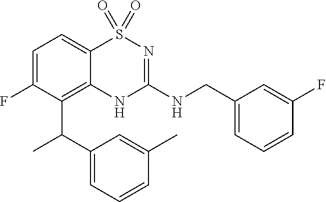 [SMILES: Cc1cccc(C(C)c2c(F)ccc3c2NC(NCc2cccc(F)c2)=NS3(=O)=O)c1] | 0.557 | 441.1 | 442.1 | [M + H]⁺ | 1 |
| 22-13 | 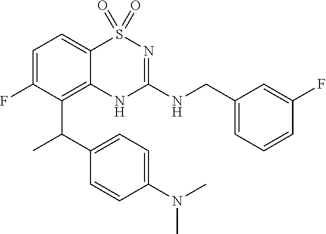 [SMILES: CC(c1ccc(N(C)C)cc1)c1c(F)ccc2c1NC(NCc1cccc(F)c1)=NS2(=O)=O] | 0.456 | 470.2 | 471.2 | [M + H]⁺ | 1 |
| 22-14 | 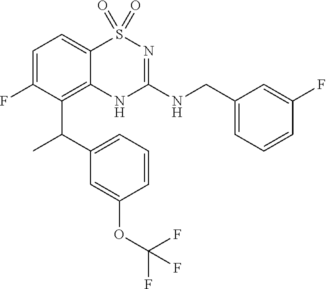 [SMILES: CC(c1cccc(OC(F)(F)F)c1)c1c(F)ccc2c1NC(NCc1cccc(F)c1)=NS2(=O)=O] | 0.573 | 511.1 | 512.1 | [M + H]⁺ | 1 |
| 22-15 | 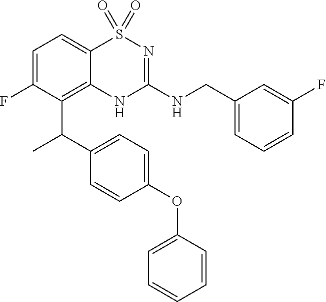 [SMILES: CC(c1ccc(Oc2ccccc2)cc1)c1c(F)ccc2c1NC(NCc1cccc(F)c1)=NS2(=O)=O] | 0.588 | 519.1 | 520.2 | [M + H]⁺ | 1 |
| 22-16 | 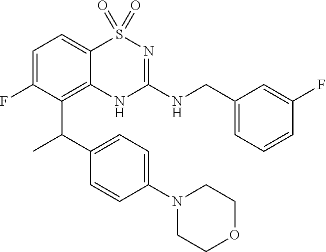 [SMILES: CC(c1ccc(N2CCOCC2)cc1)c1c(F)ccc2c1NC(NCc1cccc(F)c1)=NS2(=O)=O] | 0.511 | 512.2 | 513.2 | [M + H]⁺ | 1 |
| 22-17 | 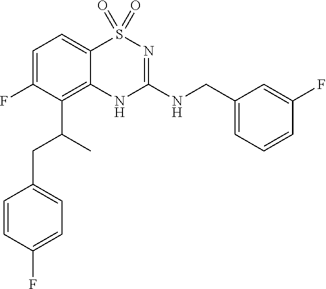 [SMILES: CC(Cc1ccc(F)cc1)c1c(F)ccc2c1NC(NCc1cccc(F)c1)=NS2(=O)=O] | 0.549 | 459.1 | 460.1 | [M + H]⁺ | 1 |
| 22-18 | 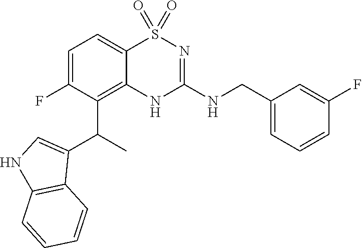 [SMILES: CC(c1c(F)ccc2c1NC(NCc1cccc(F)c1)=NS2(=O)=O)c1c[nH]c2ccccc12] | 0.530 | 466.1 | 467.1 | [M + H]⁺ | 1 |
| 22-19 | 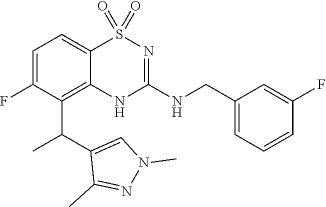 [SMILES: Cc1nn(C)cc1C(C)c1c(F)ccc2c1NC(NCc1cccc(F)c1)=NS2(=O)=O] | 0.470 | 445.1 | 446.2 | [M + H]⁺ | 1 |
| 22-20 | 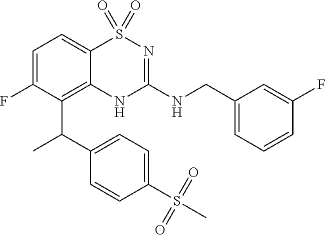 [SMILES: CC(c1ccc(S(C)(=O)=O)cc1)c1c(F)ccc2c1NC(NCc1cccc(F)c1)=NS2(=O)=O] | 0.496 | 505.1 | 506.1 | [M + H]⁺ | 1 |
| 22-21 | 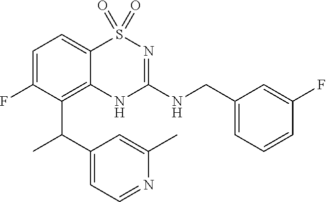 [SMILES: Cc1cc(C(C)c2c(F)ccc3c2NC(NCc2cccc(F)c2)=NS3(=O)=O)ccn1] | 0.428 | 442.1 | 443.1 | [M + H]⁺ | 1 |
| 22-22 | 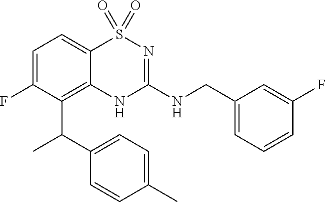 [SMILES: Cc1ccc(C(C)c2c(F)ccc3c2NC(NCc2cccc(F)c2)=NS3(=O)=O)cc1] | 0.559 | 441.1 | 442.1 | [M + H]⁺ | 1 |
| 22-23 | 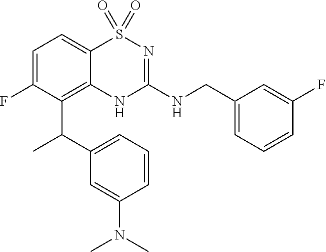 [SMILES: CC(c1cccc(N(C)C)c1)c1c(F)ccc2c1NC(NCc1cccc(F)c1)=NS2(=O)=O] | 0.461 | 470.2 | 471.1 | [M + H]⁺ | 1 |
| 22-24 | 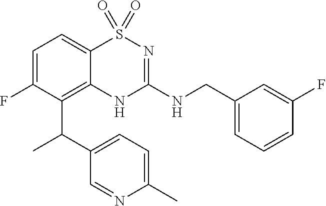 [SMILES: Cc1ccc(C(C)c2c(F)ccc3c2NC(NCc2cccc(F)c2)=NS3(=O)=O)cn1] | 0.426 | 442.1 | 443.1 | [M + H]⁺ | 1 |
| 22-25 | 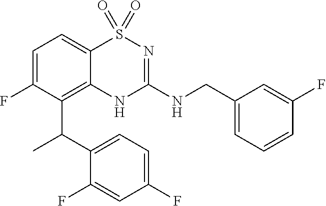 [SMILES: CC(c1ccc(F)cc1F)c1c(F)ccc2c1NC(NCc1cccc(F)c1)=NS2(=O)=O] | 0.545 | 463.1 | 464.1 | [M + H]⁺ | 1 |
| 22-26 | 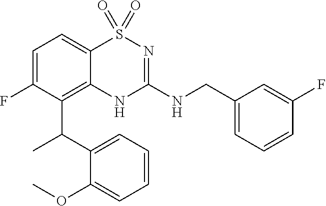 [SMILES: COc1ccccc1C(C)c1c(F)ccc2c1NC(NCc1cccc(F)c1)=NS2(=O)=O] | 0.542 | 457.1 | 458.2 | [M + H]⁺ | 1 |
| 22-27 | 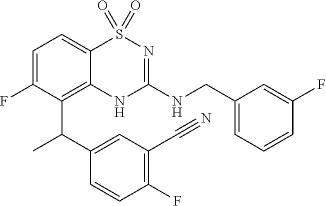 [SMILES: CC(c1ccc(F)c(C#N)c1)c1c(F)ccc2c1NC(NCc1cccc(F)c1)=NS2(=O)=O] | 0.521 | 470.1 | 471.1 | [M + H]⁺ | 1 |
| 22-28 | 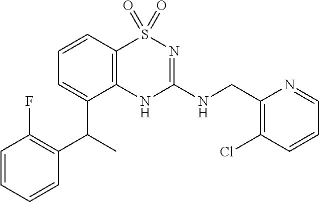 [SMILES: CC(c1ccccc1F)c1cccc2c1NC(NCc1ncccc1Cl)=NS2(=O)=O] | 0.524 | 444.1 | 445.1 | [M + H]⁺ | 1 |
| 22-29 | 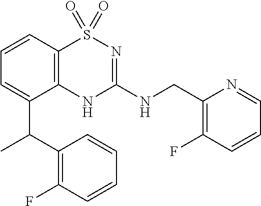 [SMILES: CC(c1ccccc1F)c1cccc2c1NC(NCc1ncccc1F)=NS2(=O)=O] | 0.507 | 428.1 | 429.2 | [M + H]⁺ | 1 |

### Example 23

### Synthesis of Representative Compound 23-1

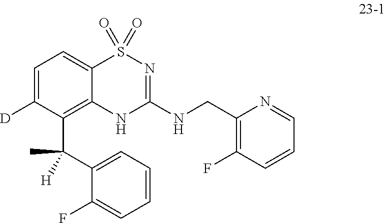 [SMILES: [2H]c1ccc2c(c1[C@H](C)c1ccccc1F)NC(NCc1ncccc1F)=NS2(=O)=O]

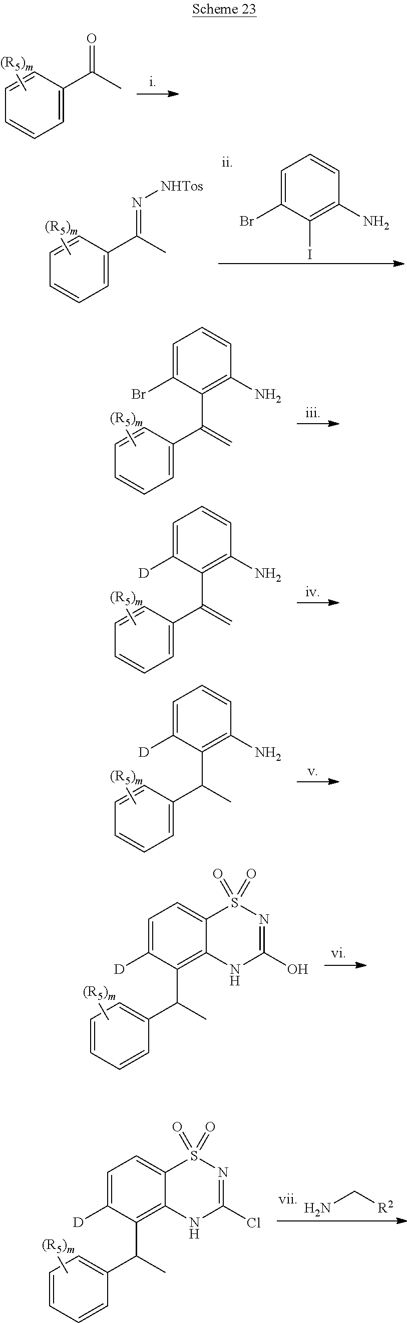

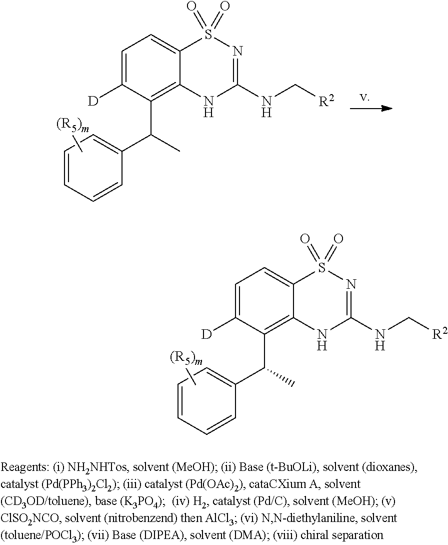

### Example Synthesis of Scheme 23 Compounds

### Step 23-1. Synthesis of 3-bromo-2-(1-(2-fluorophenyl)vinyl)aniline (INT 23A)

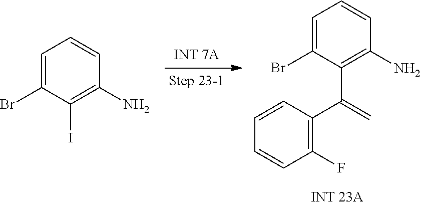 [SMILES: Nc1cccc(Br)c1I ; C=C(c1ccccc1F)c1c(N)cccc1Br]

To a mixture of 3-bromo-2-iodo-aniline (3.0 g, 10.1 mmol, 1 eq), N-[(E)-1-(2-fluorophenyl)ethylideneamino]-4-methyl-benzenesulfonamide INT 7A (3.0 g, 9.79 mmol, 9.72e-1 eq), and t-BuOLi (1.5 g, 18.7 mmol, 1.7 mL, 1.9 eq) in dioxane (100 mL) was added Pd(PPh₃)₂Cl₂ (700 mg, 997 mol, 9.90e-2 eq). The reaction mixture was heated at 100° C. for 15 h. The reaction was quenched with water (100 mL) and the aqueous layer was extracted with ethyl acetate (3×100 mL). The combined organic layers were dried over anhydrous sodium sulfate, filtered, and concentrated in vacuo to afford crude residue. The residue was purified by flash silica gel chromatography (0-20% ethyl acetate/petroleum ether gradient at 50 mL/min) to afford 3-bromo-2-(1-(2-fluorophenyl)vinyl)aniline INT 23A (2.2 g, 4.59 mmol, 46% yield) that was used without further purification. LCMS (m/z) calculated for C₁₄H₁₁BrFN: 291.0; found 292.0 [M+H]⁺, t_{R}=0.583 min (Purity Method 1). ¹H NMR (400 MHz, CHLOROFORM-d) δ=7.27-7.20 (m, 1H), 7.14-7.07 (m, 2H), 7.05-6.98 (m, 3H), 6.69 (dd, J=1.5, 7.5 Hz, 1H), 6.20 (s, 1H), 5.61 (s, 1H), 3.91 (br s, 2H).

### Step 23-2. Synthesis of 2-(1-(2-fluorophenyl)vinyl)benzen-3-d-amine (INT 23B)

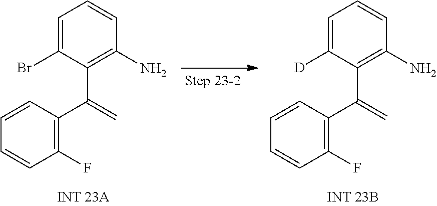 [SMILES: C=C(c1ccccc1F)c1c(N)cccc1Br ; [2H]c1cccc(N)c1C(=C)c1ccccc1F]

To a mixture of 3-bromo-2-(1-(2-fluorophenyl)vinyl)aniline INT 23A (2.2 g, 4.6 mmol, 1 eq), bis(1-adamantyl)-butylphosphane (61.0 mg, 170 mol, 3.70e-2 eq) and K₃PO₄ (1.53 g, 7.18 mmol, 1.56 eq) in CD₃OD (20 mL) and toluene (80 mL) was added Pd(OAc)₂ (61.0 mg, 272 mol, 5.91e-2 eq). The reaction mixture was heated at 50° C. for 15 h. The mixture was filtered and concentrated in vacuo. The residue was purified by flash silica gel chromatography (−0-20% ethyl acetate/petroleum ether gradient at 50 mL/min) to afford the product with 78% purity. The material was further purified by prep-HPLC (column: Phenomenex luna C18 150×40 mm×15 um; A-B mobile phase: [water(FA)-ACN]; gradient: 45%-75% B over 15 min) to afford 2-(1-(2-fluorophenyl)vinyl)benzen-3-d-amine INT 23B (600 mg, 2.38 mmol, 52% yield) that was used without further purification. LCMS (m/z) calculated for C₁₄H₁₁DFN: 214.1; found 215.0 [M+H]⁺, t_{R}=0.518 min (Purity Method 1). ¹H NMR (400 MHz, METHANOL-d4) δ=7.35-7.25 (m, 1H), 7.22-7.15 (m, 1H), 7.13-7.04 (m, 3H), 6.75 (dd, J=1.1, 8.0 Hz, 1H), 6.71-6.64 (m, 1H), 5.78 (t, J=1.4 Hz, 1H), 5.54 (t, J=1.4 Hz, 1H).

### Step 23-3. Synthesis of 2-(1-(2-fluorophenyl)ethyl)benzen-3-d-amine (INT 23C)

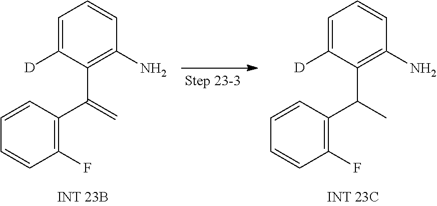 [SMILES: [2H]c1cccc(N)c1C(=C)c1ccccc1F ; [2H]c1cccc(N)c1C(C)c1ccccc1F]

A mixture of 2-(1-(2-fluorophenyl)vinyl)benzen-3-d-amine INT 23B (600 mg, 2.80 mmol, 1 eq) and Pd/C (100 mg, 10% purity) in MeOH (30 mL) was degassed and purged with H2 3 times, and then the mixture was stirred at 50° C. for 15 h under an H2 atmosphere (50 psi). The mixture was filtered and the filtrate was concentrated in vacuo to afford 2-(1-(2-fluorophenyl)ethyl)benzen-3-d-amine INT 23C (540 mg, 2.42 mmol, 86% yield). LCMS (m/z) calculated for C₁₄H₁₃DFN: 216.1; found 217.2 [M+H]⁺, t_{R}=0.678 min (Purity Method 2). ¹H NMR (400 MHz, METHANOL-d4) δ=7.25-6.95 (m, 5H), 6.80-6.68 (m, 2H), 4.44 (q, J=7.0 Hz, 1H), 1.58 (d, J=7.1 Hz, 3H).

### Step 23-4. Synthesis of 5-(1-(2-fluorophenyl)ethyl)-3-hydroxy-4H-benzo[e][1,2,4]thiadiazine 1,1-dioxide-6-d (INT 23D)

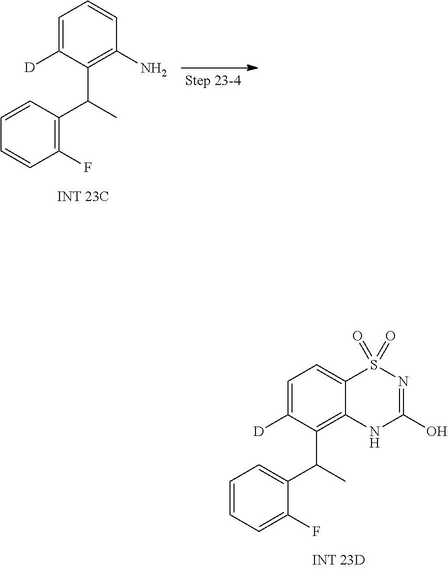 [SMILES: [2H]c1cccc(N)c1C(C)c1ccccc1F ; [2H]c1ccc2c(c1C(C)c1ccccc1F)NC(O)=NS2(=O)=O]

To a mixture of 2-(1-(2-fluorophenyl)ethyl)benzen-3-d-amine INT 23C (350 mg, 1.62 mmol, 1 eq) in nitrobenzene (5.3 mL) was added N-(oxomethylene)sulfamoyl chloride (270 mg, 1.91 mmol, 166 μL, 1.18 eq) at 0° C., and it was stirred for 0.5 h. Then, to the mixture was added AlCl₃ (270 mg, 2.02 mmol, 111 μL, 1.25 eq), and it was heated at 80° C. for 4 h. Combined a previous batch to do the work-up. The reaction mixture was slowly poured into 50 mL saturated aqueous NH₄Cl solution, then extracted with ethyl acetate (3×30 mL). The combined organics were dried over anhydrous sodium sulfate, filtered, and concentrated in vacuo. To the residue was added 200 mL petroleum ether and the product precipitated. The precipitate was filtered, washed with petroleum ether and dried under vacuum to afford 5-(1-(2-fluorophenyl)ethyl)-3-hydroxy-4H-benzo[e][1,2,4]thiadiazine 1,1-dioxide-6-d INT 23D (800 mg, 1.49 mmol, 92% yield) that was used without further purification. LCMS (m/z) calculated for C₁₅H₁₂DFN₂O₃S: 321.1; found 320.1 [M−H], t_{R}=0.447 min (Purity Method 2 negative mode).

### Step 23-5. Synthesis of 3-chloro-5-(1-(2-fluorophenyl)ethyl)-4H-benzo[e][1,2,4]thiadiazine 1,1-dioxide-6-d (INT 23E)

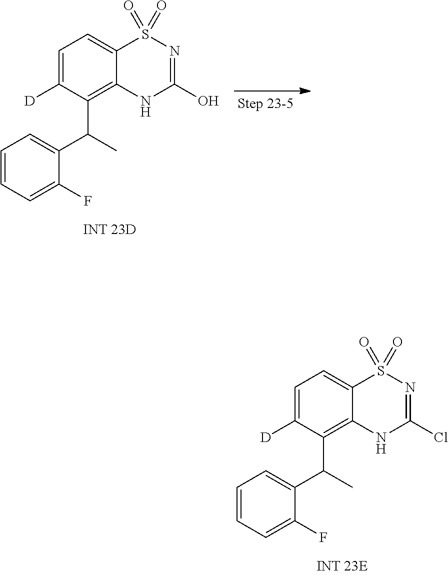 [SMILES: [2H]c1ccc2c(c1C(C)c1ccccc1F)NC(O)=NS2(=O)=O ; [2H]c1ccc2c(c1C(C)c1ccccc1F)NC(Cl)=NS2(=O)=O]

To a mixture of 5-(1-(2-fluorophenyl)ethyl)-3-hydroxy-4H-benzo[e][1,2,4]-thiadiazine 1,1-dioxide-6-d INT 23D (400 mg, 747 mol, 1 eq) and N,N-diethylaniline (170 mg, 1.14 mmol, 182 μL, 1.53 eq) in toluene (10 mL) was added POCl₃ (2.29 g, 14.9 mmol, 1.39 mL, 20 eq). The reaction mixture was heated at 110° C. for 15 h. The reaction mixture was slowly quenched by adding 50 mL of aqueous saturated NaHCO₃ solution. The aqueous layer was extracted with ethyl acetate (3×30 mL). The combined organics were dried over anhydrous sodium sulfate, filtered, and concentrated in vacuo. The residue was purified by flash silica gel chromatography (0-100% ethyl acetate/petroleum ether gradient at 40 mL/min) to afford 3-chloro-5-(1-(2-fluorophenyl)ethyl)-4H-benzo[e][1,2,4]thiadiazine 1,1-dioxide-6-d INT 23E (200 mg, 512 mol, 69% yield) that was used without further purification. LCMS (m/z) calculated for C₁₅H₁₁DClFN₂O₂S: 339.0; found 338.0 [M−H], t_{R}=0.538 min (Purity Method 2 negative mode).

### Step 23-6. Synthesis of (S)-5-(1-(2-fluorophenyl)ethyl)-3-(((3-fluoropyridin-2-yl)methyl)amino)-4H-benzo[e][1,2,4]thiadiazine 1,1-dioxide-6-d (23-1)

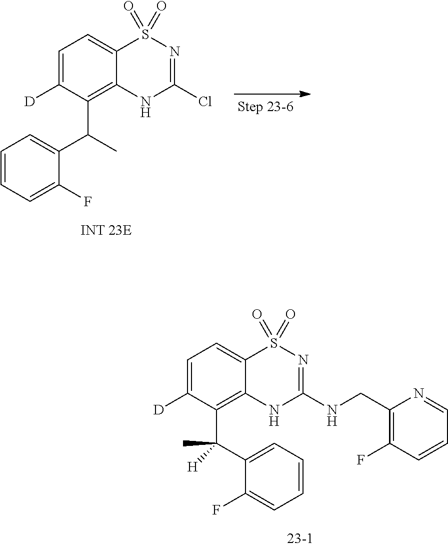 [SMILES: [2H]c1ccc2c(c1C(C)c1ccccc1F)NC(Cl)=NS2(=O)=O ; [2H]c1ccc2c(c1[C@H](C)c1ccccc1F)NC(NCc1ncccc1F)=NS2(=O)=O]

To a mixture of 3-chloro-5-(1-(2-fluorophenyl)ethyl)-4H-benzo[e][1,2,4]thiadiazine 1,1-dioxide-6-d INT 23E (200 mg, 512 mol, 1 eq) and DIPEA (100 mg, 774 mol, 135 μL, 1.51 eq) in DMA (10 mL) was added (3-fluoro-2-pyridyl)methanamine (131 mg, 656 mol, 1.28 eq, 2HCl). The reaction mixture was heated at 120° C. for 15 h. The mixture was added 10 mL water and extracted with ethyl acetate (3×15 mL). The combined organics were dried over anhydrous sodium sulfate, filtered, and concentrated in vacuo. The residue was purified by prep-HPLC (column: C18 150×30 mm; A-B mobile phase: [water(FA)-ACN]; gradient: 42%-72% B over 7 min) to afford 120 mg racemic. The racemic product was further separated by SFC (column: DAICEL CHIRALCEL OD (250 mm×30 mm, 10 μm); A-B mobile phase: [CO₂-MeOH(0.1% NH₃H₂O)]; B %: 30%, isocratic elution mode) to afford two enantiomers. (S)-5-(1-(2-fluorophenyl)ethyl)-3-(((3-fluoropyridin-2-yl)methyl)amino)-4H-benzo[e][1,2,4]-thiadiazine 1,1-dioxide-6-d 23-1 (47.4 mg, 110 mol, 22% yield). LCMS (m/z) calculated for C₂₁H₁₇DClF₂N₄O₂S: 429.1; found 430.2 [M+H]⁺, t_{R}=0.470 min (Purity Method 1). t_{R}=1.348 min (Chiral Purity Method 1). ¹H NMR (400 MHz, DMSO-d6) δ=10.26 (br s, 1H), 8.48 (d, J=4.6 Hz, 1H), 8.11 (br s, 1H), 7.80 (t, J=9.3 Hz, 1H), 7.61-7.45 (m, 3H), 7.40-7.31 (m, 1H), 7.30-7.23 (m, 1H), 7.22-7.09 (m, 2H), 4.78-4.72 (m, 1H), 4.70 (br s, 2H), 1.59 (d, J=7.0 Hz, 3H) and (R)-5-(1-(2-fluorophenyl)ethyl)-3-(((3-fluoropyridin-2-yl)methyl)amino)-4H-benzo[e]-[1,2,4]thiadiazine 1,1-dioxide-6-d 23-2 (2.6 mg, 1% yield). LCMS (m/z) calculated for C₂₁H₁₇DClF₂N₄O₂S: 429.1; found 430.2 [M+H]⁺, t_{R}=0.470 min (Purity Method 1).

The compounds listed in Table 24 were made using the procedures of Scheme 23.

TABLE 24

| | | Purity | Exact | | | |
|---|---|---|---|---|---|---|
| Cpd | | RT | mass | observed | | Purity |
| No. | Structure | (min) | (g/mol) | m/z | Ion | Method |
| 23-1 | 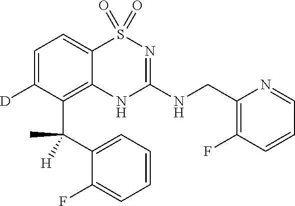 [SMILES: [2H]c1ccc2c(c1[C@H](C)c1ccccc1F)NC(NCc1ncccc1F)=NS2(=O)=O] | 0.468 | 429.1 | 430.1 | [M + H]⁺ | 1 |
| 23-2 | 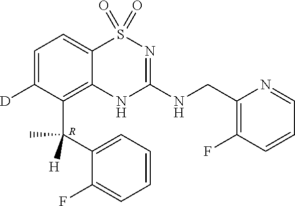 | 0.469 | 429.1 | 430.1 | [M + H]⁺ | 1 |

### Example 24

### Synthesis of Representative Compound 24-1

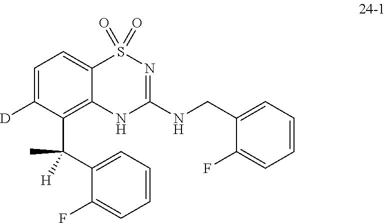 [SMILES: [2H]c1ccc2c(c1[C@H](C)c1ccccc1F)NC(NCc1ccccc1F)=NS2(=O)=O]

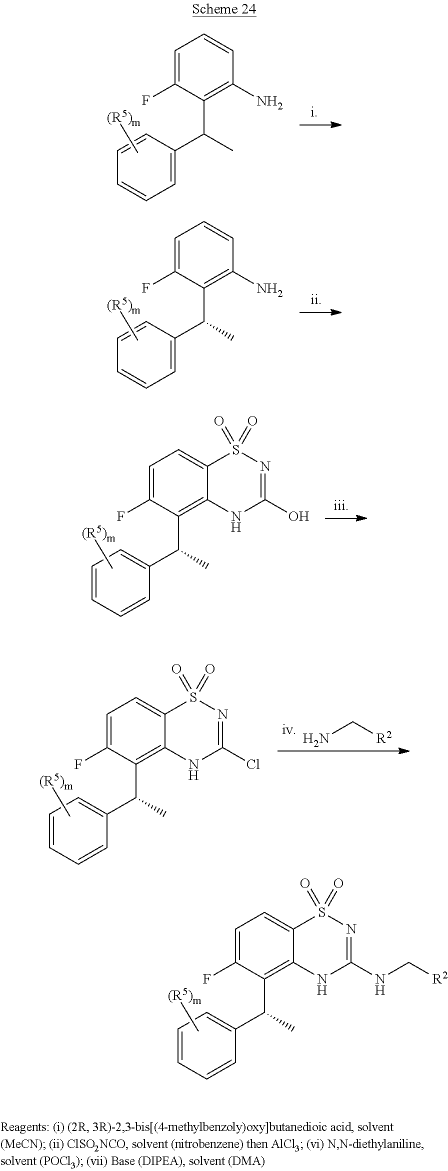

### Step 24-1. Synthesis of (R)-3-fluoro-2-(1-(2-fluorophenyl)ethyl)aniline (INT 24A)

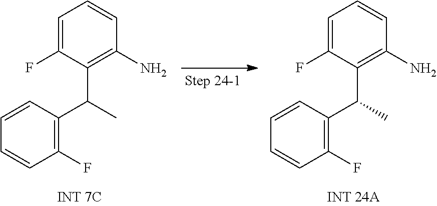 [SMILES: CC(c1ccccc1F)c1c(N)cccc1F ; C[C@H](c1ccccc1F)c1c(N)cccc1F]

To a solution of 3-fluoro-2-[1-(2-fluorophenyl)ethyl]aniline INT 7C (7.2 g, 27.2 mmol, 1 eq) in MeCN (100 mL) was added (2R,3R)-2,3-bis[(4-methylbenzoyl)oxy]butanedioic acid (4.73 g, 12.3 mmol, 0.45 eq). The mixture was stirred at 25° C. for 1 h. The mixture was then heated to 70° C. and stirred for 1 h. The mixture was cooled to 25° C. and stirred at 25° C. for 15 h. The reaction mixture was filtered and the solid was dried under reduced pressure. To the collected solid was added water (30 mL) and extracted with ethyl acetate (2×20 mL). The organic phase was dried over anhydrous sodium sulfate, filtered, and concentrated in vacuo to afford (R)-3-fluoro-2-(1-(2-fluorophenyl)ethyl)aniline INT 24A (1.5 g, 6.43 mmol, 24% yield). The filtrate was concentrated under reduced pressure to give a residue. To the residue was added water (30 mL) and the aqueous was extracted with ethyl acetate (2×20 mL). The organic phase was dried over anhydrous sodium sulfate, filtered, and concentrated in vacuo to afford 4.5 g of crude (INT 24A). To a solution of crude INT 24A (4.5 g, 17.3 mmol, 1 eq) in MeCN (63 mL) was added (2R,3R)-2,3-bis[(4-methylbenzoyl)oxy]butanedioic acid (2.00 g, 5.18 mmol, 0.3 eq). The mixture was stirred at 25° C. for 1 h. The mixture was then heated to 70° C. and stirred for 1 h. The mixture was cooled to 25° C. and stirred at 25° C. for 15 h. The reaction mixture was filtered and the solid was dried under vacuum. To the collected solid was added water (30 mL) and extracted with ethyl acetate (2×20 mL). The organics were dried over anhydrous sodium sulfate, filtered, and concentrated in vacuo to afford additional (R)-3-fluoro-2-(1-(2-fluorophenyl)ethyl)aniline INT 24A (1.4 g, 5.97 mmol, 35% yield). LCMS (m/z) calculated for C₁₄H₁₃F₂N: 233.1; found 234.1 [M+H]⁺, t_{R}=0.556 min (Purity Method 1); 99.2% ee, t_{R}=1.457 min (Chiral Purity Method 1).

### Step 24-2. Synthesis of (R)-6-fluoro-5-(1-(2-fluorophenyl)ethyl)-3-hydroxy-4H-benzo[e][1,2,4]thiadiazine 1,1-dioxide (INT 24B)

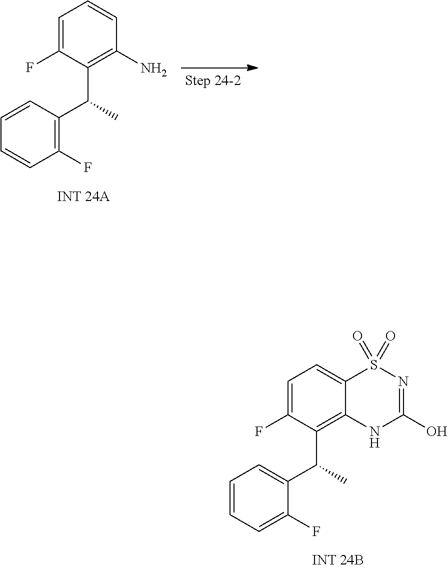 [SMILES: C[C@H](c1ccccc1F)c1c(N)cccc1F ; C[C@H](c1ccccc1F)c1c(F)ccc2c1NC(O)=NS2(=O)=O]

To a solution of (R)-3-fluoro-2-(1-(2-fluorophenyl)ethyl)aniline INT 24A (1.4 g, 6.00 mmol, 1 eq) in nitrobenzene (20 mL), was added N-(oxomethylene)sulfamoyl chloride (1.27 g, 9.00 mmol, 782 μL, 1.5 eq) at 0° C. The mixture was stirred at 0° C. for 0.5 h. Then, AlCl₃ (1.00 g, 7.50 mmol, 410 μL, 1.25 eq) was added. The mixture was stirred at 80° C. for 1.5 h. The reaction solution was cooled to room temperature and then diluted with aqueous saturated NH₄Cl solution (60 mL). The aqueous phase was extracted with ethyl acetate (3×60 mL). The combined organics were washed with brine (50 mL), dried over anhydrous sodium sulfate, filtered, and concentrated in vacuo to give a brown oil. The obtained brown oil was diluted with petroleum ether (200 mL) and then stirred for 30 min. The brown oil eventually formed a brown precipitate that was collected by vacuum filtration. The filter cake was washed with petroleum ether (200 mL) and then dried under vacuum to give a brown solid that was triturated in toluene (10 mL) and collected by filtration to afford (R)-6-fluoro-5-(1-(2-fluorophenyl)ethyl)-3-hydroxy-4H-benzo[e][1,2,4]thiadiazine 1,1-dioxide INT 24B (4 g, crude) that was used without further purification. LCMS (m/z) calculated for C₁₅H₁₂F₂N₂O₃S: 338.1; found 337.0 [M−H], t_{R}=0.445 min (Purity Method 2 negative mode)

### Step 24-3. Synthesis of (R)-3-chloro-6-fluoro-5-(1-(2-fluorophenyl)ethyl)-4H-benzo[e][1,2,4]thiadiazine 1,1-dioxide (INT 24C)

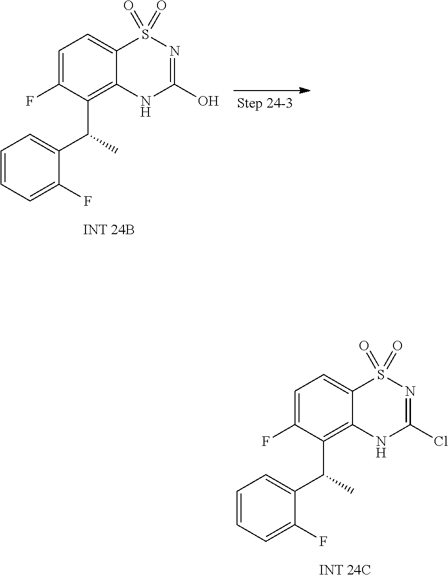 [SMILES: C[C@H](c1ccccc1F)c1c(F)ccc2c1NC(O)=NS2(=O)=O ; C[C@H](c1ccccc1F)c1c(F)ccc2c1NC(Cl)=NS2(=O)=O]

To a solution of POCl₃ (18.0 g, 117 mmol, 10.9 mL, 15 eq) in toluene (50 mL), was added (R)-6-fluoro-5-(1-(2-fluorophenyl)ethyl)-3-hydroxy-4H-benzo[e][1,2,4]thiadiazine 1,1-dioxide INT 24B (4 g, crude) N,N-diethylaniline (1.75 g, 11.7 mmol, 1.87 mL, 1.5 eq). The mixture was stirred at 110° C. for 16 h. The reaction mixture was poured into water (100 mL) portionwise while the temperature was kept below 35° C. The obtained mixture was stirred for 1 hr. The aqueous layer was extracted with EtOAc (3×70 mL). The combined organics were washed with brine (2×50 mL), dried over Na₂SO₄, filtered, and concentrated under vacuum to give a residue. The residue was purified by flash silica gel chromatography (0-50% ethyl acetate/petroleum ether gradient at 80 mL/min) to afford (R)-3-chloro-6-fluoro-5-(1-(2-fluorophenyl)ethyl)-4H-benzo[e][1,2,4]thiadiazine 1,1-dioxide INT 24C (1.2 g, 3.30 mmol, 42% yield). LCMS (m/z) calculated for C₁₅H₁₁ClF₂N₂O₂S: 356.0; found 355.0 [M−H], t_{R}=0.553 min (Purity Method 2 negative mode).

### Step 24-4. Synthesis of (R)-6-fluoro-3-((2-fluorobenzyl)amino)-5-(1-(2-fluorophenyl)ethyl)-4H-benzo[e][1,2,4]thiadiazine 1,1-dioxide (24-1)

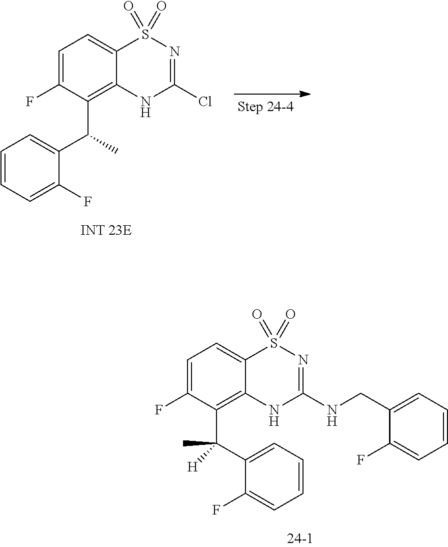 [SMILES: C[C@H](c1ccccc1F)c1c(F)ccc2c1NC(Cl)=NS2(=O)=O ; C[C@H](c1ccccc1F)c1c(F)ccc2c1NC(NCc1ccccc1F)=NS2(=O)=O]

To a solution of (R)-3-chloro-6-fluoro-5-(1-(2-fluorophenyl)ethyl)-4H-benzo[e][1,2,4]thiadiazine 1,1-dioxide INT 24C (70 mg, 161 mol, 1 eq), (2-fluorophenyl)methanamine (30.2 mg, 241 mol, 28 μL, 1.5 eq) in DMA (1.5 mL) was added DIPEA (85.2 mg, 659 mol, 115 μL, 4.10 eq). The mixture was heated at 120° C. for 14 h. The crude mixture was filtered and the filtrate was purified by preparative HPLC (column: Phenomenex luna C18 150×25 mm×10 um; A-B mobile phase: [water(FA)-ACN]; gradient: 45%-75% B over 1 min) to afford (R)-6-fluoro-3-((2-fluorobenzyl)amino)-5-(1-(2-fluorophenyl)ethyl)-4H-benzo[e][1,2,4]thiadiazine 1,1-dioxide 24-1 (26.4 mg, 58.9 mol, 37% yield). ¹HNMR (400 MHz, DMSO-d6) δ=10.26 (br s, 1H), 7.71 (br s, 1H), 7.64 (br dd, J=5.5, 8.5 Hz, 1H), 7.55 (br t, J=7.8 Hz, 1H), 7.46 (br t, J=7.4 Hz, 1H), 7.43-7.35 (m, 1H), 7.35-7.21 (m, 4H), 7.16-7.06 (m, 1H), 7.00 (br dd, J=9.1, 10.7 Hz, 1H), 4.74-4.42 (m, 3H), 1.69 (br d, J=6.9 Hz, 3H). LCMS (m/z) calculated for C₂₂H₁₈F₃N₃O₂S: 445.1; found 446.1 [M+H]⁺, t_{R}=0.538 min (Purity Method 1); 100% ee, t_{R}=1.221 min (Chiral Purity Method 2).

The compounds listed in Table 25 were made using the procedures of Scheme 24.

TABLE 25

| | | | Exact | | | |
|---|---|---|---|---|---|---|
| Cpd | | Purity | mass | observed | | Purity |
| No. | Structure | RT (min) | (g/mol) | m/z | Ion | Method |
| 24-1 | 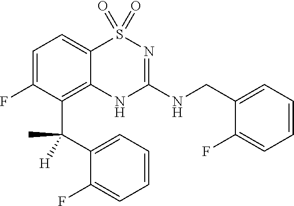 [SMILES: C[C@H](c1ccccc1F)c1c(F)ccc2c1NC(NCc1ccccc1F)=NS2(=O)=O] | 0.540 | 445.1 | 446.1 | [M + H]⁺ | 1 |
| 24-2 | 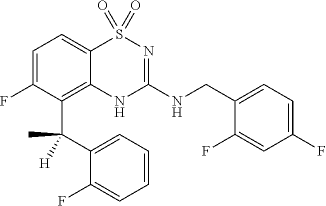 [SMILES: C[C@H](c1ccccc1F)c1c(F)ccc2c1NC(NCc1ccc(F)cc1F)=NS2(=O)=O] | 0.534 | 463.1 | 464.1 | [M + H]⁺ | 1 |
| 24-3 | 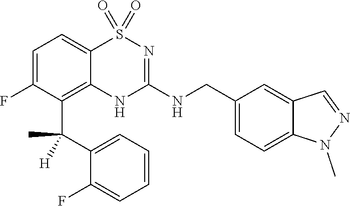 [SMILES: C[C@H](c1ccccc1F)c1c(F)ccc2c1NC(NCc1ccc3c(cnn3C)c1)=NS2(=O)=O] | 0.516 | 481.1 | 482.1 | [M + H]⁺ | 1 |
| 24-4 | 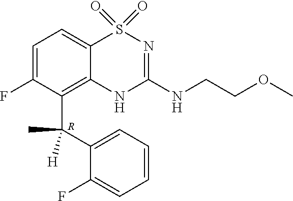 | 7.574 | 395.1 | 396.2 | [M + H]⁺ | 3 |
| 24-5 | 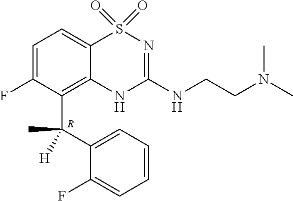 | 6.348 | 408.1 | 409.2 | [M + H]⁺ | 3 |
| 24-6 | 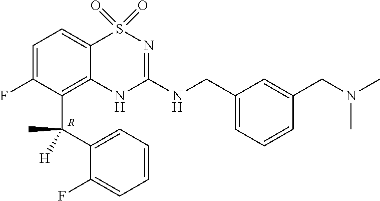 | 6.031 | 484.2 | 485.2 | [M + H]⁺ | 3 |
| 24-7 | 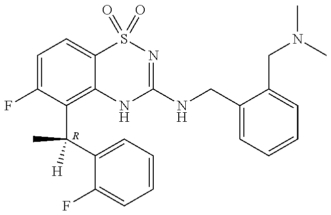 | 6.236 | 484.2 | 485.2 | [M + H]⁺ | 3 |
| 24-8 | 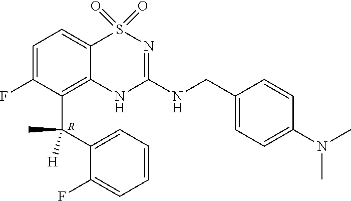 | 6.821 | 470.2 | 471.2 | [M + H]⁺ | 3 |

### Example 25

### MRGPRX2 Activity

CHO cells stably transfected to express human MRGPRX2 were maintained in an incubator at 37° C. with 5% CO₂ and grown in F12 (HAM) media with 10% fetal bovine serum (FBS), 1% Glutamax, 1% penicillin/streptomycin, 800 μg/mL Geneticin (G418), and 300 μg/mL Hygromycin B.

Cells were plated in a 384-well assay plate at 20,000 cells per well in 12 μL of Opti-MEM and kept in an incubator overnight. On the day of the assay, compounds solubilized at 10 mM in DMSO were added as a 10-point curve (30 uM final top concentration with 1:3 serial dilutions) using a Tecan D300E digital dispenser. Agonists were diluted in assay buffer (final concentrations of 5.7 mM Tris-HCl, 43 mM NaCl, 50 mM LiCl, pH=8) and 2 μL of the agonist Cortistatin-14 (CPC Scientific, catalog CORT-002) are added to each well. Final concentrations of agonists were 0.3 μM Cortistatin. Final concentrations of DMSO were kept consistent across the plate. Plates were incubated in the dark for 1 h at 37° C. and then for 1 h at room temperature. IP-1 standards and HTRF detection reagents were added according to the IP-One—Gq Kit purchased from Cisbio (part number 62IPAPEJ) and incubated in the dark for 1 h at room temperature. The plate was read on a Molecular Devices SpectraMax iD5 plate reader. The HTRF ratio was calculated from the raw data and graphed using GraphPad Prism to calculate an IC50 value for each compound.

Activity data for selected MRGPRX2 antagonists (versus 0.3 uM Cortistatin-14 agonist) are displayed in Table 26. The activity ranges are denoted as follows: “+++++” denotes antagonist activity <100 nM; “++++” denotes antagonist activity between 100 and 500 nM; “+++” denotes activity between 501 and 1000 nM; “++” denotes activity between 1001 and 2500 nM; and “+” denotes activity >2500 nM.

TABLE 26

| Cpd. | MRGPRX2 |
|---|---|
| No | Antagonist Activity |
| 1-1 | ++++ |
| 1-2 | +++++ |
| 1-3 | ++++ |
| 1-4 | +++++ |
| 1-5 | +++++ |
| 1-6 | +++++ |
| 1-7 | ++++ |
| 1-8 | +++++ |
| 1-9 | +++++ |
| 1-10 | +++++ |
| 1-11 | +++++ |
| 1-12 | ++++ |
| 1-13 | + |
| 1-14 | +++++ |
| 1-15 | ++++ |
| 1-16 | ++++ |
| 1-17 | ++++ |
| 1-18 | +++++ |
| 1-19 | +++++ |
| 1-20 | ++ |
| 1-21 | +++ |
| 1-22 | ++++ |
| 1-23 | +++++ |
| 1-24 | +++++ |
| 1-25 | +++++ |
| 1-26 | ++ |
| 1-27 | +++++ |
| 1-28 | +++ |
| 1-29 | +++++ |
| 1-30 | ++++ |
| 1-31 | ++++ |
| 1-32 | ++++ |
| 1-33 | +++ |
| 1-34 | +++++ |
| 1-35 | ++ |
| 1-36 | +++++ |
| 1-37 | + |
| 1-38 | +++++ |
| 1-39 | +++++ |
| 1-40 | ++++ |
| 1-41 | ++++ |
| 1-42 | ++++ |
| 1-43 | +++++ |
| 1-44 | ++++ |
| 1-45 | ++++ |
| 1-46 | +++++ |
| 1-47 | +++ |
| 1-48 | +++++ |
| 1-49 | +++++ |
| 1-50 | +++++ |
| 1-51 | +++++ |
| 1-52 | + |
| 1-244 | ++ |
| 1-264 | +++++ |
| 2-1 | +++++ |
| 2-2 | +++++ |
| 2-3 | + |
| 2-4 | +++++ |
| 2-5 | ++++ |
| 2-6 | ++++ |
| 2-7 | ++++ |
| 2-8 | ++++ |
| 2-9 | ++++ |
| 2-10 | ++++ |
| 2-11 | ++ |
| 2-12 | ++ |
| 2-13 | +++ |
| 3-1 | +++++ |
| 3-2 | +++++ |
| 4-1 | +++++ |
| 4-2 | ++++ |
| 4-3 | +++++ |
| 4-4 | ++++ |
| 4-5 | ++++ |
| 4-6 | ++++ |
| 4-7 | ++ |
| 4-8 | ++++ |
| 4-9 | +++ |
| 4-10 | ++++ |
| 4-11 | ++++ |
| 4-12 | ++++ |
| 4-13 | +++ |
| 4-14 | ++++ |
| 4-15 | + |
| 4-16 | ++++ |
| 4-17 | ++++ |
| 4-18 | + |
| 4-19 | +++ |
| 4-20 | +++ |
| 5-1 | +++++ |
| 6-2 | +++++ |
| 6-3 | +++++ |
| 6-4 | +++++ |
| 6-5 | +++++ |
| 6-6 | +++++ |
| 6-7 | +++++ |
| 6-8 | +++++ |
| 6-9 | +++++ |
| 6-10 | +++++ |
| 6-11 | +++++ |
| 6-12 | +++++ |
| 6-13 | ++++ |
| 6-14 | +++++ |
| 6-15 | ++++ |
| 6-16 | ++++ |
| 6-17 | +++++ |
| 6-1 | ++++ |
| 6-18 | +++++ |
| 6-19 | +++++ |
| 7-1 | +++++ |
| 7-2 | +++++ |
| 7-3 | ++++ |
| 7-4 | +++++ |
| 7-5 | +++++ |
| 7-6 | +++++ |
| 7-7 | +++++ |
| 7-8 | +++++ |
| 7-9 | +++++ |
| 8-1 | +++++ |
| 8-2 | +++++ |
| 8-3 | +++++ |
| 9-1 | +++++ |
| 9-2 | +++++ |
| 9-3 | +++++ |
| 9-4 | +++++ |
| 9-5 | +++++ |
| 9-6 | +++++ |
| 9-7 | +++++ |
| 10-1A | +++++ |
| 10-1B | +++++ |
| 10-2A | +++++ |
| 10-2B | +++++ |
| 10-3A | +++++ |
| 10-3B | +++++ |
| 10-4A | +++++ |
| 10-4B | +++++ |
| 10-5A | +++++ |
| 10-5B | +++++ |
| 10-6A | +++++ |
| 10-6B | ++++ |
| 11-1 | ++++ |
| 12-1 | +++++ |
| 12-2 | +++++ |
| 12-3 | +++++ |
| 12-4 | +++++ |
| 12-5 | +++++ |
| 12-6 | +++++ |
| 12-7 | +++++ |
| 12-8 | +++++ |
| 12-9 | +++++ |
| 12-10 | +++++ |
| 12-11 | +++++ |
| 12-12 | +++++ |
| 12-13 | +++++ |
| 12-14 | +++++ |
| 12-15 | ++++ |
| 12-16 | +++++ |
| 12-17 | +++++ |
| 12-18 | +++++ |
| 12-19 | +++++ |
| 12-20 | +++++ |
| 12-21 | +++++ |
| 12-22 | +++++ |
| 12-23 | +++++ |
| 12-24 | +++++ |
| 12-25 | +++++ |
| 12-26 | ++ |
| 12-27 | +++++ |
| 12-28 | +++++ |
| 12-29 | +++++ |
| 12-30 | +++++ |
| 12-31 | +++++ |
| 12-32 | +++++ |
| 13-1 | +++++ |
| 13-2 | +++++ |
| 13-3 | +++++ |
| 14-1 | +++++ |
| 14-2 | ++++ |
| 14-3 | +++++ |
| 15-1 | +++ |
| 16-1 | +++++ |
| 16-2 | +++++ |
| 16-3 | ++ |
| 16-4 | +++++ |
| 16-5 | ++++ |
| 16-6 | ++++ |
| 17-1 | + |
| 18-1 | ++++ |
| 19-1 | ++++ |
| 20-1 | +++++ |
| 21-1 | +++++ |
| 22-1 | +++++ |
| 22-2 | +++++ |
| 22-3 | +++++ |
| 22-4 | +++++ |
| 22-5 | +++++ |
| 22-6 | +++++ |
| 22-7 | +++++ |
| 22-8 | ++++ |
| 22-9 | ++++ |
| 22-10 | +++++ |
| 22-11 | +++++ |
| 22-12 | +++++ |
| 22-13 | ++++ |
| 22-14 | ++++ |
| 22-15 | +++ |
| 22-16 | ++ |
| 22-17 | +++++ |
| 22-18 | ++++ |
| 22-19 | +++++ |
| 22-20 | ++++ |
| 22-21 | +++++ |
| 22-22 | ++++ |
| 22-23 | ++++ |
| 22-24 | +++++ |
| 22-25 | +++++ |
| 22-26 | +++++ |
| 22-27 | +++++ |
| 23-1 | +++++ |
| 24-1 | +++++ |
| 24-2 | +++++ |
| 24-3 | +++++ |

### Example 26

### Mast Cell Beta-Hexosaminidase Release Assay

Human LAD2 cells (NIH) were maintained in an incubator at 37° C. with 5% CO₂ and cultured in StemPro-34 serum-free media (Gibco 10639011) supplemented with 2 mM L-glutamine, 100 U/ml penicillin, 50 mg/ml streptomycin and 100 ng/ml SCF (Invitrogen PEP0860), at a concentration of 2-5×10⁵ cells/mL, with hemidepletion every 1-2 weeks.

Cells were transferred to SCF-Free medium at 2.5×10⁵ cells/mL and kept in an incubator overnight. On the day of the assay, cells were washed twice in Assay Buffer (final concentrations 10 mM HEPES, 137 mM NaCl, 5.6 mM D-glucose, 2.7 mM KCl, 1 mM MgCl, 1.8 mM CalCl₂, 0.4 mM Na₂HPO₄·7H₂O, 0.04% BSA, pH=7.4) and plated in a 96-well v-bottom plate at 20,000 cells per well in 80 μL Assay Buffer. Antagonist compounds solubilized at 10 mM in DMSO were diluted in Assay Buffer to 10× final desired concentrations as a 10-point curve (10 μM final top concentration with 1:3 serial dilutions) and 10 μL added per well. Plates were then incubated for 1 hour at 37° C. Agonists were diluted in Assay Buffer to 10× desired concentrations and 10 μL of the appropriate agonist added to each well. The final concentration of Cortistatin-14 (Tocris 3374) used in antagonist assays was 500 nM. Final concentrations of DMSO were kept consistent across the plate. Plates were then incubated in a warm air oven for 30 minutes at 37° C., followed by centrifugation at 4° C. for 5 minutes at 450×g. 50 μL supernatant from each well was then transferred to a 96-well flat bottom plate containing 100 μL substrate solution per well (3.5 mg/mL p-Nitrophenyl-N-acetyl-o-D-glucosaminide (Sigma 487052) in Citrate Buffer containing a final concentration of 40 mM Citric Acid, 20 mM Na₂HPO₄·7H₂O, pH=4.5). To the cell pellets left in the remaining assay buffer, 150 μL 0.1% Triton-X-100 was then added to each well, resuspended by pipetting up and down, and 50 μL cell lysates transferred to a second 96-well flat bottom plate containing 100 μL substrate solution per well. Plates containing transferred supernatant and cell lysates were then incubated in a warm air oven for 90 minutes at 37° C. After incubation, 50 μL of 400 mM Glycine buffer (pH 10.7) was added into each well and the plate was read on a Molecular Devices SpectraMax iD5 plate reader (absorbance at 405 nm with reference filter at 620 nm). After background subtraction, the percentage degranulation (percent beta-hexosaminidase release) was calculated as 100×(supernatant values)/(supernatant+lysate values), followed by analysis using GraphPad Prism software to calculate an IC₅₀ value for each compound.

Activity data for selected MRGPRX2 antagonists in the Mast Cell Beta-Hexosaminidase Release Assay are displayed in Table 27. The activity ranges are denoted as follows: “+++++” denotes antagonist activity <100 nM; “++++” denotes antagonist activity between 100 and 500 nM; “+++” denotes activity between 501 and 1000 nM; “++” denotes activity between 1001 and 2500 nM; and “+” denotes activity >2500 nM.

TABLE 27

| Cpd. | MRGPRX2 Antagonist |
|---|---|
| No. | Activity |
| 1-264 | +++++ |
| 2-1 | ++++ |
| 9-1 | +++++ |
| 10-1A | ++++ |
| 10-1B | +++++ |
| 10-2A | +++ |
| 10-2B | +++++ |
| 10-3A | ++++ |
| 10-3B | +++++ |
| 10-4A | ++++ |
| 10-4B | +++++ |
| 10-5A | ++ |
| 10-5B | +++++ |
| 10-6A | +++++ |
| 10-6B | ++++ |

### Example 27

### Mouse Pharmacokinetic Studies

Compounds were formulated in 5% DMSO, 5% Solutol, and 90% PBS without Ca and Mg (pH 7.4) at a concentration of 0.5 mg/mL. Male C57BL/6 mice (n=6/compound) were administered a 50 mg/kg dose of each compound by oral gavage under a non-fasted condition. Blood samples were collected via the Submandibular vein onto K2-EDTA at 0.5, 1, 2, 4, 8 and 24 hours after dosing, and plasma was prepared and stored at −80° C. until analysis. Plasma sample preparation for analysis was done by protein precipitation using acetonitrile (including Verapamil as an internal standard) followed by centrifugation. Compound concentrations were determined in extracted plasma using LC-MS/MS relative to a 12-point standard curve covering the 1 to 4000 nM range. Non-compartmental analysis using Phoenix WinNonlin was used to estimate pharmacokinetic parameters including area under the curve, clearance, and half-life. The administered dose was confirmed by analysis of residual dosing material by UPLC-UV relative to a single point calibration sample. The results of these studies are presented in Table 28.

TABLE 28

| Cpd. No. | Cmax (μM) | TBC (mL/min/kg) | T_{1/2} (h) |
|---|---|---|---|
| 1-264 | 1.1 | 32 | 3.8 |
| 2-1 | 11 | 8.8 | 2.8 |
| 9-1 | 8.7 | 13 | 1.8 |
| 10-1A | 4.6 | 26 | 2.6 |
| 10-1B | 14 | 9 | 1.8 |
| 10-2A | 2.7 | 30 | 3.0 |
| 10-2B | 4.4 | 27 | 2.3 |
| 10-3A | 0.37 | 4.3 | 2.5 |
| 10-3B | 4.2 | 41 | 2 |
| 10-4A | 9.6 | 11 | 4.4 |
| 10-4B | 10 | 12 | 2.2 |
| 10-5A | 2.8 | 50 | 2.2 |
| 10-5B | 7.1 | 12 | 2.5 |
| 10-6A | 11 | 11 | 2.1 |
| 10-6B | 2.7 | 33 | — |

### Example 28

### Solubility Studies

Media according to Biorelevant Media protocols was prepared. 10 μL of 10 mM in DMSO stock solutions of test compounds was added to 96-well deep plate. 490 μL of FaSSIF, FeSSIF, or FaSSGF was then added into appropriate wells of 96-well deep plate. The plate was vortexed for 2 minutes then placed on a plate shaker for 24 hours at room temperature. The plate was centrifuged for 20 minutes, then supernatant was collected and filtered with 450 micron syringe filter into UPLC vials for injection into UPLC-UV system. Concentration was calculated with a 3-point standard curve. Results are shown in Table 29.

TABLE 29

| Cpd. No. | FaSSIF (μM) | FeSSIF (μM) | FaSSGF (μM) |
|---|---|---|---|
| 1-264 | <12.5 | <12.5 | <12.5 |
| 2-1 | <12.5 | <12.5 | <12.5 |
| 9-1 | 114 | 74 | 19 |
| 10-1B | 133 | 200 | 61 |
| 10-2B | 162 | 186 | 11 |
| 10-4B | 23 | 37 | 3 |
| 10-5B | 158 | 136 | <3.1 |
| 10-6A | 30 | 178 | 27 |

### Example 29

### MRGPRX2 FLIPR Calcium Mobilization Assay

Chinese hamster ovary (CHO-K1) cells stably expressing human MRGPRX2 are purchased from Genescript (Piscataway, NJ). The cells are maintained in culture medium (Ham's F-12K) containing 10% (v/v) FBS, 200 μg/mL Zeocin, 100 units/mL penicillin G and 100 μg/mL streptomycin (Life Technologies, Carlsbad, CA). For the assay, the cells are harvested and resuspended with culture medium without Zeocin before plating at 8000 cells per well in 20 μL in 384-well black clear bottom cell culture plates (VWR, Radnor, PA). After 24 hour culture at 37° C. and 5% CO₂, the cells are loaded with 20 μL/well of calcium dye (FLIPR Calcium 6 Assay Kit, Molecular Devices, San Jose, CA) diluted in loading buffer (1× Hank's Balanced Salt Solution (HBSS), 5 mM probenecid, 20 mM 4-(2-hydroxyethyl)-1-piperazineethanesulfonic acid (HEPES), pH 7.4, VWR, Radnor, PA) followed with 45 min. incubation at 37° C. and 40 min. at r.t. in the dark. Cells are then treated with the addition of 10 uL/well test compounds with a range of increasing concentrations diluted in the assay buffer (1×HBSS, 20 mM HEPES, pH 7.4, VWR, Radnor, PA) with FLIPR Penta (Molecular Devices. San Jose, CA) and incubated for 30 min. at r.t. Ca²⁺ release was measured with FLIPR Penta with 10 seconds basal fluorescence measurement, then 12.5 μL of 5× agonist Cortistatin-14 (Tocris, Minneapolis, MN) at final concentration corresponding to the EC80 are added followed with continued fluorescence signal monitoring for an additional 110 seconds. The base line adjusted (median of first 10 s base line) max value of the Relative Fluorescence Unit (RFU) is plotted against compound concentrations. Wells with no compound serve as the positive controls, and wells with high concentration of reference antagonist are used as negative controls. IC₅₀ curves are globally fitted with 3- or 4-parameter Hill equation in a Genedata Screener (Genedata Basel, Switzerland).

### Example 30

### MRGPRX2 IP1 HTRF Assay

This assay measures compound inhibition of myo-Inositol 1 phosphate (IP1) accumulation in CHO-K1 cells. Chinese hamster ovary (CHO-K1) cells stably expressing human MRGPRX2 are purchased from GenScript (Piscataway, NJ).

For the HTRF IP1 determination, an IP-One GqKit (Perkin Elmer Cisbio, Waltham, MA), which includes all reagents and buffers in this protocol, are used. The cells are maintained in culture medium (Ham's F-12K) containing 10% (v/v) FBS, and 200 μg/mL Zeocin. For the assay, the cells are harvested and resuspended in culture medium with 2% FBS and without Zeocin, then passed through a 40 μm filter. Cells are added at 20000 cells in a 5 μL/well to a 384-well white small volume cell culture plate (Greiner VWR, Radnor, PA) which contains 50 nL/well of test compound serially diluted at a selected concentration range in DMSO. 5 μL/well of prepared 2× Stimulation Buffer2 is then added to plates and incubated at 37° C. with 5% CO₂ for 1 h. 5 μL/well of agonist Cortistatin 14 (Bio-TechneR&D Systems, Minneapolis, MN) in 1× Stimulation Buffer 2, for a 1 μM final concentration, is then added to plates and incubated at 37° C. with 5% CO₂ for 1 h. 6 μL/well of 1:20 diluted Detection Reagent Mix (d2 and Cryptate) is added to the plates and incubated for 1 h in the dark. Plates are read on the PHERAstar microplate reader (BMG Labtech Cary, NC) to determine the HTRF ratio of the acceptor and donor emission signals. The IP1 concentrations are calculated from a standard curve following the IP-One GQ kit instructions. Wells with DMSO only and wells with high concentration of reference antagonist are used as controls for normalization. Compound IC₅₀ curves are globally fitted with 3- or 4-parameter Hill equation in a Genedata Screener (Genedata Basel, Switzerland).

### Example 31

### MRGPRX2 β-Arrestin Assay

This assay measures compound inhibition of P-Arrestin recruitment, which is part of the G protein-independent pathway that results from the ligand-activated GPCR phosphorylation by specific GPCR kinases. The PathHunter CHO-K1 MRPGRX2 B-Arrestin Cell line stably expressing ProLink tagged MRGPRX2 and Enzyme Acceptor Tagged B-Arrestin are purchased from Eurofins DiscoverX, (Fremont, CA).

The cells are maintained in culture medium from the Europhins DiscoverX Cell Culture Kit-107 which includes FBS, hygromycin B and G418. For the assay, the cells are harvested and were resuspended with Cell Plating Reagent 2 (Eurofins DiscoverX). Cells are added 10000 cells in 25 μL/well to a 384-well black cell culture plate (Greiner VWR, Radnor, PA) which contained 125 nL/well of compound at a selected serially diluted concentration range or DMSO. The plate is incubated at 37° C. with 5% CO₂ for 1 h. 2 μL/well of agonist Cortistatin 14 is diluted in Protein Dilution Buffer, (Eurofins DiscoverX) for a final concentration of 0.25 μM, is added and the plate incubated at 37° C. with 5% CO₂ for 90 min. 14 μL/well of Detection Reagent Mix (PathHunter Detection Kit, Eurofins DiscoverX) is added to the plates and further incubated for 1 h in the dark. Plates are read on the PHERAstar microplate reader (BMG Labtech Cary, NC) measuring luminescence 0.1 to 1 second per well. Data is normalized using DMSO only wells and wells with high concentration of reference antagonist as controls. Compound IC₅₀ curves are globally fitted with 3- or 4-parameter Hill equation in a Genedata Screener (Genedata Basel, Switzerland).

### Example 32

### Caco2 Assay

Caco-2 cells are grown at 37° C. in an atmosphere of 5% CO₂ in DMEM growth medium supplemented with 10% (v/v) fetal bovine serum, 1% (v/v) nonessential amino acids, penicillin (100 U/*mL), and streptomycin (100 μg/*mL). Confluent cell monolayers are subcultured every 7 days or 4 days for Caco-2 by treatment with 0.05% trypsin containing 1 μM EDTA. Caco-2 cells are seeded in 96-well Transwell plates. The seeding density for Caco-2 cells is 14,000 cells/well. DMEM growth medium is replaced every other day after seeding. Cell monolayers are used for transport assays between 22 and 25 days for Caco-2 cells.

Cell culture medium is removed and replaced with HBSS. To measure the TEER, the HBSS is added into the donor compartment (apical side) and receiver compartment (basolateral side). The TEER is measured by using a REMS Autosampler to ensure the integrity of the cell monolayers. Caco-2 cell monolayers with TEER values ≥300 Ω·cm² are used for transport experiments. To determine the Pₐₚₚ in the absorptive direction (A-B), solution of test compound (50 M) in HBSS is added to the donor compartment (apical side), while HBSS solution with 4% BSA is added to the receiver compartment (basolateral side). The apical volume was 0.075 mL, and the basolateral volume is 0.25 mL. The incubation period is 120 min. at 37° C. in an atmosphere of 5% CO₂. At the end of the incubation period, samples from the donor and receiver sides are removed and an equal volume of MeCN is added for protein precipitation. The supernatants are collected after centrifugation (3000 rpm, Allegra X-14R Centrifuge from Beckman Coulter, Indianapolis, IN) for LCMS analysis. The permeability value is determined according to the equation:

$${P}_{app} ( cm / s ) = ( F * VD ) / ( SA * MD ) ,$$

where the flux rate (F, mass/time) is calculated from the slope of cumulative amounts of compound of interest on the receiver side, SA is the surface area of the cell membrane, VD is the donor volume, and MD is the initial amount of the solution in the donor chamber.

### Example 33

### Human Whole Blood Stability

The whole blood stability of the exemplified compounds is determined by LC-MS/MS. The 96-Well Flexi-Tier™ Block (Analytical Sales & Services, Inc, Flanders, NJ) is used for the incubation plate containing 1.0 mL glass vials with 0.5 mL of blood per vial (pooled gender, human whole blood sourced from BIOIVT, Hicksville, NY or similar). Blood is pre-warmed in water bath to 37° C. for 30 min. 96-deep well analysis plate is prepared with the addition of 100 μL ultrapure water/well. 50 μL chilled ultrapure water/well is added to 96-deep well sample collection plate and covered with a sealing mat. 1 μL of 0.5 mM compound working solution (DMSO:water) is added to the blood in incubation plate to reach final concentrations of 1 μM, mixed by pipetting thoroughly and 50 μL is transferred 50 into the T=0 wells of the sample collection plate. Blood is allowed to sit in the water for 2 min. and then 400 μL stop solution/well is added (MeCN containing an internal standard). The incubation plate is placed in the Incu-Shaker CO₂ Mini incubator (Benchmark Scientific, Sayreville, NJ) at 37° C. with shaking at 150 rpm. At 1, 2 and 4-h, the blood samples are mixed thoroughly by pipetting and 50 μL is transferred into the corresponding wells of the sample collection plate. Blood is allowed to sit in the water for 2 min. and then 400 μL of stop solution/well is added. The collection plate is sealed and vortexed at 1700 rpm for 3 min. (VX-2500 Multi-Tube Vortexer, VWR International, Radnor, PA), and samples are then centrifuged in the collection plate at 3500 rpm for 10 min. (Allegra X-14R Centrifuge Beckman Coulter, Indianapolis, IN). 100 μL of supernatant/well is transferred from the sample collection plate into the corresponding wells of the analysis plate. The final plate is vortexed at 1700 rpm for 1 min. and analyze samples by LC-MS/MS. The peak area ratio of the 1, 2, and 4 h samples relative to T=0 is used to determine the percent remaining. The natural log of the percent remaining versus time is used determine a slope to calculate the compounds half-life in blood (ti/2=0.693/slope).

### Example 34

### In Vitro Intrinsic Clearance Protocol

For in vitro metabolic stability experiments, test compounds are incubated with human liver microsomes at 37° C. The incubation mixture contains test compounds (1 PM), NADPH (2 mM), and human liver microsomes (0.5 mg protein/*mL) in 100 mM phosphate buffer (pH 7.4). The mixture is pre-incubated for 2 min. at 37° C. before the addition of NADPH. Reactions are commenced upon the addition of NADPH and quenched with ice-cold MeOH at 0, 10, 20, and 30 min. Terminated incubation mixtures are analyzed using LC-MS/MS system. The analytical system consisted of a Shimadzu LC-30AD binary pump system and SIL-30AC autosampler (Shimadzu Scientific Instruments, Columbia, MD) coupled with a Sciex Triple Quad 6500+mass spectrometer from Applied Biosystems (Foster City, CA). Chromatographic separation of test compounds and internal standard is achieved using a Hypersil Gold C18 column (50×2.1 mm, 5 μM, 175 A) from ThermoFisher Scientific (Waltham, MA). Mobile phase A consists of 0.1% formic acid in water, and mobile phase B consists of 0.1% formic acid in MeCN. The total LC-MS/MS runtime can be 2.75 min. with a flow rate of 0.75 mL/min. Peak area integrations and peak area ratio calculations are performed using Analyst software (version 1.6.3) from Applied Biosystems.

The in vitro intrinsic clearance, CL_{int, in vitro}, is calculated from the t_{1/2} of test compound disappearance as CL_{int, in vitro}=(0.693/t_{1/2})×(1/Cₚᵣₒₜₑᵢₙ), where Cₚᵣₒₜₑᵢₙ is the protein concentration during the incubation, and t_{1/2} is determined by the slope (k) of the log-linear regression analysis of the concentration versus time profiles; thus, t_{1/2}=ln 2/k. The CL_{int, in vitro} values are scaled to the in vivo values for human by using physiologically based scaling factors, hepatic microsomal protein concentrations (45 mg protein/g liver), and liver weights (21 g/kg body weight). The equation CLᵢₙₜ=CL_{int, in vitro}×(*mg protein/g liver weight)×(g liver weight/kg body weight) is used. The in vivo hepatic clearance (CLH) is then calculated by using CLᵢₙₜ and hepatic blood flow, Q (20 mL·min.⁻¹·kg⁻¹ in humans) in the well-stirred liver model disregarding all binding from CL_{H}=(Q×CLᵢₙₜ)/(Q+CLᵢₙₜ). The hepatic extraction ratio is calculated as CLH divided by Q.

### Example 35

### In Vivo Pharmacokinetics Protocol

For in vivo pharmacokinetic experiments, test compounds are administered to male Sprague Dawley rats or male and female Cynomolgus monkeys intravenously or via oral gavage. For intravenous (IV) dosing, test compounds are dosed at 0.5 to 1 mg/kg using a formulation of 10% dimethylacetamide (DMAC) in acidified saline via IV bolus for rat and 5 min. or 10 min. IV infusion for monkey. For oral (PO) dosing, test compounds are dosed at 1.0 to 3.0 mg/kg using 5% DMAC in 0.5% methylcellulose in citrate buffer (pH 2.5). Blood samples are collected at predose and various time points up to 24 h postdose. All blood samples are collected using EDTA as the anticoagulant and centrifuged to obtain plasma samples. The plasma concentrations of test compounds are determined by LC-MS methods. The measured plasma concentrations are used to calculate PK parameters by standard noncompartmental methods using Phoenix® WinNonlin software program (version 8.0, Pharsight Corporation).

In rats and monkeys, cassette dosing of test compounds are conducted to obtain preliminary PK parameters.

In vivo pharmacokinetic experiments with male beagle dogs may be performed under the conditions described above.

### Example 36

### Time Dependent Inhibition (TDI) of CYP Protocol

This assay is designed to characterize an increase in CYP inhibition as a test compound is metabolized over time. Potential mechanisms for this include the formation of a tight-binding, quasi-irreversible inhibitory metabolite complex or the inactivation of P450 enzymes by covalent adduct formation of metabolites. While this experiment employs a 10-fold dilution to diminish metabolite concentrations and therefore effects of reversible inhibition, it is possible (but not common) that a metabolite that is an extremely potent CYP inhibitor could result in a positive result.

The results are from a cocktail of CYP specific probe substrates at 4 times their Km concentrations for CYP2C9, 2C19, 2D6 and 3A4 (midazolam) using human liver microsomes (HLM). The HLMs can be pre-incubated with test compounds at a concentration 10 μM for 30 min. in the presence (+N) or absence (−N) of a NADPH regenerating system, diluted 10-fold, and incubated for 8 min. in the presence of the substrate cocktail with the addition of a fresh aliquot of NADPH regenerating system. A calibration curve of metabolite standards can be used to quantitatively measure the enzyme activity using LC-MS/MS. In addition, incubations with known time dependent inhibitors, tienilic aicd (CYP2C9), ticlopidine (CYP2C19), paroxetine (CYP2D6), and troleandomycin (CYP3A4), used as positive controls are pre-incubated 30 min. with or without a NADPH regenerating system.

The analytical system consists of a Shimadzu LC-30AD binary pump system and SIL-30AC autosampler (Shimadzu Scientific Instruments, Columbia, MD) coupled with a Sciex Triple Quad 6500+mass spectrometer from Applied Biosystems (Foster City, CA). Chromatographic separation of test compounds and internal standard can be achieved using an ACQUITY UPLC BEH 130A, 2.1×50 mm, 1.7 μm HPLC column (Waters Corp, Milford, MA). Mobile phase A consists of 0.1% formic acid in water, and mobile phase B consists of 0.1% formic acid in MeCN. The total LC-MS/MS runtime will be 2.50 min. with a flow rate of 0.9 mL/min. Peak area integrations and peak area ratio calculations are performed using Analyst software (version 1.6.3) from Applied Biosystems.

The percentage of control CYP2C9, CYP2C19, CYP2D6, and CYP3A4 activity remaining following preincubation of the compounds with NADPH is corrected for the corresponding control vehicle activity and then calculated based on 0 min. as 100%. A linear regression plot of the natural log of % activity remaining versus time for each isozyme is used to calculate the slope. The −slope is equal to the rate of enzyme loss, or the K_{obs}.

### Example 37

### In vitro Degranulation Assay—β-Hexosaminidase Release Assay

The assay is performed essentially as described by Hermans, et al., Front. Immunol., 2021, 12, 625284. Mature mast cells are cultured in StemPro-34 medium with StemPro-34 nutrient supplement (Gibco), 2 mM L-glutamine (Gibco), 100 U/mL penicillin/100 μg/mL streptomycin (Gibco), 100 ng/mL recombinant human stem cell factor (PeproTech) and 100 ng/mL recombinant human interleukin 6 (Peprotech). The cells are washed in HEPES-buffered Tyrode's solution (Thermo Fisher) containing 0.1% (w/v) bovine serum albumine (further called “releasing medium”) and seeded at 1×10⁵ cells per well in 96-well plate in the releasing medium. Selected wells are pretreated with predefined concentrations of MRGPRX2 antagonist for 1 h at 37° C. with 5% CO₂. Then, about 10 μg/mL compound 48/80 (poly-p-methoxyphenethylmethylamine) or about 30 μM of substance P is added to treated wells and equal amount of releasing medium is added into negative control wells. Following 1 h incubation, the plate is centrifuged for 5 min. and 50 μL of supernatant is collected into a new 96-well plate. In a positive control group, remaining supernatant is removed and 100 μL 0.1% Triton X-100 (v/v) is added to lyse the cell at 300 g for 5 min. After that, the plate is centrifuged and 50 μL of the lysate is also transferred into the new plate. Supernatant and lysate samples are then incubated with 50 μL of p-nitrophenyl N-acetyle-B-D-glucosaminide in 0.1 M citrate buffer pH 4-4.5 (Thermo Fisher) for 1 h at 37° C. The enzymatic reaction is stopped by the addition of 100 μL/well of 0.1 M Na₂CO₃ buffer pH 10 (Thermo Fisher). The absorbance is read at 405 nm. Net 0-hexosaminidase release (%) is calculated as =[(stimulated release-spontaneous release)/total content in lysed cells]×100.

### Example 38

### Evaluation of the MRGPRX2 Antagonist Effects on In Vitro-Derived Human Mast Cells

This assay evaluates the effects of example compound on preventing mast cell degranulation via the MRGPRX2 receptor. The effects of example compounds on mast cell degranulation induced by Substance P (MRGPRX2 pathway), or IgE/anti-IgE is investigated by measuring P-hexosaminidase and mast cell specific cytokine/chemokine release into the culture media. The addition of IgE/anti-IgE serves as a differentiation for the mechanism of action of the test compound.

Mature connective tissue-type mast cells (CTMCs) are plated at 50,000 cells by well in 50 μL and stimulated as follows.

### IgE Sensitization (Day 0)

All CTMCs are pre-treated with 100 μM IgE before treatment to coat the FcMR1 receptors with IgE to resemble in vitro mast cells. In this study, CTMCs are sensitized 48 h prior to pre-treatment with example compounds and degranulation (Day 2).

### Pre-Treatment (Day 0)

Omalizumab is added 4 h after IgE sensitization (IgE containing media is removed after 4 h and replaced with culture media containing omalizumab and kept for 48 h) and for the following 48 h before degranulation induction.

### Pre-Treatment (Day 2)

Test compounds and vehicles are added 48 h post IgE sensitization/omalizumab treatment for 30 min. at Day 2 of culture.

Degranulation (Day 2+30 mins)

The CTMCs are stimulated with Substance P, Compound 48/80, anti-IgE, or Tyrode's buffer on Day 2 of culture.

### β-Hexosaminidase Assay to Assess Mast-Cell Degranulation

For the P-hexosaminidase assay, 50 μL of cell supernatant is collected in each well 45 min. after degranulation treatment. Supernatant is collected on n=3 wells per condition. 0-Hexosaminidase is a potent inflammatory mediator stored in mast cells and is released by activated mast cells. The determination of P-hexosaminidase is used to evaluate the level of mast cell degranulation. The assay is a colorimetric assay measuring the 4-nitrophenol production using a multimode plate reader. See J. Karhausen, et al., J. Clin. Invest., 2016, 126(10), 3981-98.

### Cytokine Release Assay

For the cytokine release assay, 60 μL of cell supernatant is collected in each well 8 h after degranulation treatment, an anti-protease cocktail added, and frozen at −80° C. until used. Supernatant is collected on n=3 wells per condition. Cytokine release into the culture medium is measured using a custom V-PLEX Plus Human Cytokine Kit (MesoScale Discovery): TNF-α, IL-13, GM-CSF, VEGF-A, MCP-1, TL-6, IL-4, TL-5, IL-10, and IL-8. Culture media is diluted as follows:
Plate 1: Chemokine Panel 1 (human) kit-ref K15047 (panel MCP-1)-1:10;
Plate 2: Proinflammatory Panel 1 (human) kit-ref K15049 (panel IL-4, IL-6, IL-8, IL-10, IL-13, TNF-α)-1:5;
Plate 3: Cytokine Panel 1 (human) kit-ref K15050 (panel GM-CSF, IL-5, VEGF)-1:5.

### Example 39

### Determining the Effect of Example Compounds on Cytokine/Chemokine Release from HypoSkin Models after Subcutaneous Injection of Cetrorelix or Cortistatin-14

This assay evaluates the effect of the test compounds on cytokine/chemokine release (potentially from mast cells) upon systematic pre-treatment and treatment of HypoSkin models prior to subcutaneous injection of drugs known to degranulate mast cells and cause injection site reactions (namely, Cortistatin-14 and Cetrorelix).

74 HypoSkin models of 20 mm in diameter and 10 mm total thickness with 15/20 mm diameter silicon rings are produced from 2 donors (37 models per donor) according to standard procedures and cultured with 2 mL standard HypoSkin medium. Models are maintained in standard cell culture conditions for the whole culture duration at 37° C., 5% CO₂ and water saturation, with culture medium renewed every day except during weekends.

### Systematic Treatment

Vehicle control and test compounds are added to the culture media daily, from Day 0 to Day 2. The compounds remain in the culture media upon subcutaneous injections.

### Subcutaneous Injection

100 μL of Cetrorelix, Cortistatin-14, or PBS are subcutaneously injected in to the models on Day 2.

### End of Culture and Sampling

Culture media is collected on Days 2+8 h and Day 3 (24 h post-injection) and frozen at −80° C. Sampling of HypoSkin models is performed on Day 0 and Day 3 (24 h post-injection) and processed as follows:
½ are fixed in 10% buffered formalin and processed for paraffin wax embedding; and
½ are snap frozen and stored at −80° C.

### Hematoxylin and Eosin Staining

Hematoxylin and Eosin staining is performed on one 5 μm thick skin cross section for each sample. Representative images of both the epidermis/dermis and hypodermis are taken at 40× magnification to analyze skin structure integrity and viability.

### MSD Immunoassay

Cytokine release in the culture medium is measuring using the V-PLEX Plus Human Cytokine 36-Plex Kit (K15089G, MesoScale Discovery): Eotaxin, Eotaxin-3, GM-CSF, IFN-7, IL-1α, IL-1β, Il-2, IL-4, IL-5, IL-6, IL-7, IL-8, IL-8 (HA), IL-10, IL12/IL-23p40, IL-23p70, IL-13, IL-15, IL-16, IL-17a, IL-21, IL-22, IL-23, IL-27, IL-31, IP-10, MCP-1, MCP-4, MDC, MIP-1α, MIP-1β, MIP-3α, TARC, TNF-α, TNF-β, and VEGF-A.

### Example 40

### Characterization of JAK Inhibitors MRGPRX2 Antagonists on Mast Cell Activation

The effect of the janus kinase inhibitors Povorcitinib, Ruxolitinib, and MRGPRX2 antagonists disclosed herein on the activation of mast cells in vitro is compared and characterized under different activating conditions, mimicking the in vivo disease situation. This assay determines the effect of JAK inhibition and MRGPRX2 antagonism on interference different mast cell activation pathways; the effect of JAK inhibition and MRGPRX2 antagonism in mast cells to prevent the release of mediators involved in the activation of primary human blood eosinophils and T cells.

Primary human skin mast cells (hsMC) are used to study how Povorcitinib, Ruxolitinib, and MRGPRX2 antagonists interfere with different prototype mast cell activators. The cells are obtained from individuals undergoing circumcision or breast reduction surgery by isolation from skin explants in a multi-step protocol. In vitro dose response studies using different concentration of the compounds to identify IC₅₀ values are conducted. For these studies, hsMCs are pre-incubated in the presence or absence of the compounds for 20 min., followed by stimulation with anti-IgE (FcMRI stimulation) or cortistatin-14 (MRGPRX2 agonist). Degranulation responses are measured by determination of P-hexosaminidase after 1 h at 37° C.

Next, three different concentrations of the compounds according to the determined IC₅₀ are employed to assess the spectrum of mast cell activating pathways inhibited by each compound. Therefore, hsMCs are pre-incubated in the presence or absence of the individual compounds for 20 min., followed by stimulation with five distinct activators: anti-IgE, corstatin-14 (CST), stem cell factor (SCF), complement peptides C3a, and C5a. In a parallel setup, hsMCs are pre-treated with various concentrations of cyclosporine A (CSA), to assess difference in efficacy to the test compounds. CSA, inhibiting the translocation of the transcription factor NFAT into the nucleus in MCs and other immune cells, is widely used to treat urticaria. As an alternative to CSA, another JAK or BTK inhibitor can be used. Degranulation responses are measured by determination of the P-hexosaminidase after 1 h at 37° C. In addition, cell viability and surface expression of FcMRI, MRGPRX2, cKitm C3aR/C5aR are measured by flow cytometry. Mast cells from three individual mast cell preparations are used.

To assess if treatment of mast cells with Povorcitinib, Ruxolitinib, or MRGPRX2 antagonists alters their mediator release profile and thereby their ability to activate eosinophils or T cells, hsMCs are pre-treated with the compounds for 20 min., followed by stimulation with anti-IgE, CST, SCF, C3a, or C5a for 1 h, 4 h, 8 h, and 24 h. Culture supernatants are collected and added to freshly isolated human peripheral blood eosinophils and total T cells. To achieve complete T cell activation, simultaneous activation of the TCR and costimulatory receptor with antibodies targeting CD3 and CD28 together with the mast cell supernatant is performed.

Cell activation is assessed using flow cytometry by measuring upregulation of CD69 and CD63 on eosinophils, as well as level of CD69, CD154, CD25, or CD62L on T cells. In addition, cytokines secreted by the mast cells are measured using bead-based cytokine multiplex assays (45 plex). Three individual experiments are performed using mast cells and eosinophils from three individual donors.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/588,638, filed on Oct. 6, 2023, are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1-73. (canceled)

74. A compound having the structure of Formula (III-A): 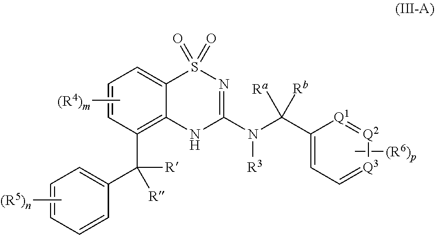
or a pharmaceutically acceptable isomer, racemate, hydrate, solvate, isotope, or salt thereof, wherein:
Q¹ is N or CR⁶, and Q² and Q³ are CR⁶;
R³ is H or C₁₋₆ alkyl;
R⁴ is, at each occurrence, independently halo, —CN, —OR⁷, —N(R⁷)₂, C₁₋₆ alkyl, C₁₋₆ haloalkyl;
R⁵ is, at each occurrence, independently halo, —CN, —OR⁷, —N(R⁷)₂, —S(O)₂—R⁷, C₁₋₆ alkyl, C₁₋₆ haloalkyl, carbocycle, or heterocycle;
R⁶ is, at each occurrence, independently halo, —CN, —OR⁷, —N(R⁷)₂, —SR⁷, —C(O)OR⁷, —N(R⁸)C(O)R⁷, —N(R⁸)C(O)OR⁷, —S(O)₂—R⁷, —C₁₋₆ alkyl-R⁷, C₁₋₆ haloalkyl, carbocycle substituted with —(R⁷)ₓ, or heterocycle substituted with —(R⁷)ₓ;
R⁷ is, at each occurrence, independently H, —OR⁸, —N(R⁸)₂, —C(O)OR⁸, —NRC(O)OR⁸, —C₁₋₆ alkyl-R⁸, C₁₋₆ haloalkyl, carbocycle substituted with —(R⁸)_{y}, or heterocycle substituted with —(R′)_{y};
R⁸ is, at each occurrence, independently H, —OR, —NR₂, —C₁₋₆ alkyl-R, carbocycle substituted with —(R)_{z}, or heterocycle substituted with —(R)_{z};
R′ and R″ are, at each occurrence, independently H, halo, cyano, hydroxy, amino, C₁₋₆ alkyl, or C₁₋₆ alkenyl, or, independently, at each occurrence of R′ and R″ attached to the same carbon atom, R′ and R″, together with the carbon to which they are attached, form a cycloalkyl;
R^{a} and R^{b} are, at each occurrence, independently H, halo, cyano, hydroxy, amino, or C₁₋₆ alkyl or, independently, at each occurrence of R^{a} and R^{b} attached to the same carbon atom, R^{a} and R^{b}, together with the carbon to which they are attached, form a cycloalkyl;
R is, at each occurrence, independently H, halo, —CN, —OH, —NH₂, or C₁₋₆ alkyl,
m is 0-5;
n is 0-5;
p is 0-5
x is 0-3;
y is 0-3; and
z is 0-3.

75. The compound of claim 74, or a pharmaceutically acceptable isomer, racemate, hydrate, solvate, isotope, or salt thereof, wherein Q¹, Q², and Q³ are each CR⁶.

76. The compound of claim 74, or a pharmaceutically acceptable isomer, racemate, hydrate, solvate, isotope, or salt thereof, wherein Q¹ is N and Q² and Q³ are each CR⁶.

77-78. (canceled)

79. The compound of claim 74, or a pharmaceutically acceptable isomer, racemate, hydrate, solvate, isotope, or salt thereof, wherein R^{a} is H.

80. The compound of claim 74, or a pharmaceutically acceptable isomer, racemate, hydrate, solvate, isotope, or salt thereof, wherein R^{a} is C₁₋₆ alkyl.

81. (canceled)

82. The compound of claim 74, or a pharmaceutically acceptable isomer, racemate, hydrate, solvate, isotope, or salt thereof, wherein R^{b} is H.

83. The compound of claim 74, or a pharmaceutically acceptable isomer, racemate, hydrate, solvate, isotope, or salt thereof, wherein R^{b} is C₁₋₆ alkyl.

84. (canceled)

85. The compound of claim 74, or a pharmaceutically acceptable isomer, racemate, hydrate, solvate, isotope, or salt thereof, wherein R^{a} and R^{b}, together with the carbon atom to which they are attached, form a cycloalkyl.

86-87. (canceled)

88. The compound of claim 74, or a pharmaceutically acceptable isomer, racemate, hydrate, solvate, isotope, or salt thereof, wherein m is 1 or 2.

89. The compound of claim 74, or a pharmaceutically acceptable isomer, racemate, hydrate, solvate, isotope, or salt thereof, wherein at least one R⁴ is C₁₋₆ alkyl.

90. (canceled)

91. The compound of claim 74, or a pharmaceutically acceptable isomer, racemate, hydrate, solvate, isotope, or salt thereof, wherein at least one R⁴ is halo.

92. The compound of claim 74, or a pharmaceutically acceptable isomer, racemate, hydrate, solvate, isotope, or salt thereof, wherein at least one R⁴ is Cl or F.

93-96. (canceled)

97. The compound of claim 74, or a pharmaceutically acceptable isomer, racemate, hydrate, solvate, isotope, or salt thereof, wherein n is 1, 2, or 3.

98. The compound of claim 74, or a pharmaceutically acceptable isomer, racemate, hydrate, solvate, isotope, or salt thereof, wherein at least one R⁵ is C₁₋₆ alkyl.

99. (canceled)

100. The compound of claim 74, or a pharmaceutically acceptable isomer, racemate, hydrate, solvate, isotope, or salt thereof, wherein at least one R⁵ is halo.

101. The compound of claim 74, or a pharmaceutically acceptable isomer, racemate, hydrate, solvate, isotope, or salt thereof, wherein at least one R⁵ is Cl or F.

102-107. (canceled)

108. The compound of claim 74, or a pharmaceutically acceptable isomer, racemate, hydrate, solvate, isotope, or salt thereof, wherein p is 1, 2, or 3.

109. The compound of claim 74, or a pharmaceutically acceptable isomer, racemate, hydrate, solvate, isotope, or salt thereof, wherein at least one R⁶ is halo.

110. The compound of claim 74, or a pharmaceutically acceptable isomer, racemate, hydrate, solvate, isotope, or salt thereof, wherein at least one R⁶ is Cl of F.

111-125. (canceled)

126. The compound of claim 74, or a pharmaceutically acceptable isomer, racemate, hydrate, solvate, isotope, or salt thereof, wherein R⁷ is H.

127. The compound of claim 74, or a pharmaceutically acceptable isomer, racemate, hydrate, solvate, isotope, or salt thereof, wherein at least one R⁶ is methyl, ethyl, propyl, or butyl.

128-141. (canceled)

142. The compound of claim 74 having one of the following structures, or a pharmaceutically acceptable isomer, racemate, hydrate, solvate, isotope, or salt thereof:

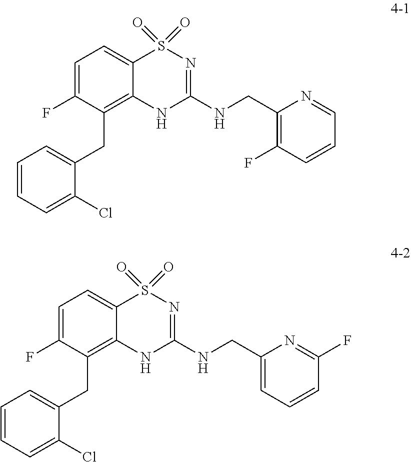 [SMILES: O=S1(=O)N=C(NCc2ncccc2F)Nc2c1ccc(F)c2Cc1ccccc1Cl ; O=S1(=O)N=C(NCc2cccc(F)n2)Nc2c1ccc(F)c2Cc1ccccc1Cl]

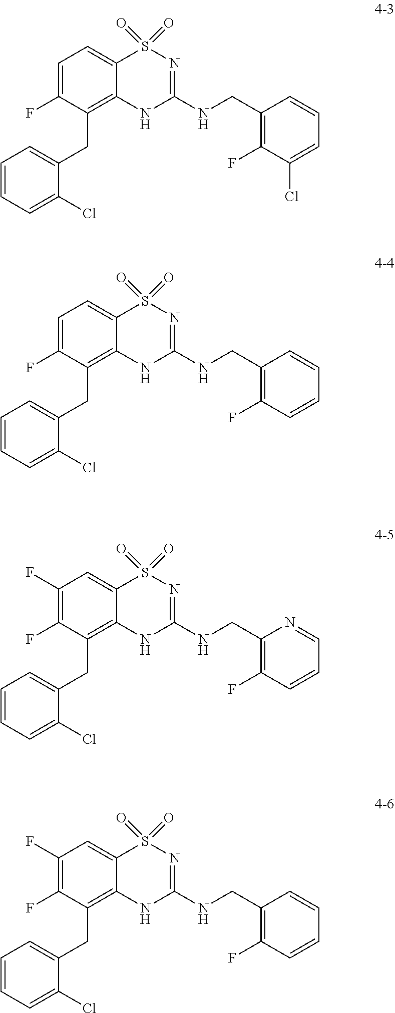 [SMILES: O=S1(=O)N=C(NCc2cccc(Cl)c2F)Nc2c1ccc(F)c2Cc1ccccc1Cl ; O=S1(=O)N=C(NCc2ccccc2F)Nc2c1ccc(F)c2Cc1ccccc1Cl ; O=S1(=O)N=C(NCc2ncccc2F)Nc2c1cc(F)c(F)c2Cc1ccccc1Cl ; O=S1(=O)N=C(NCc2ccccc2F)Nc2c1cc(F)c(F)c2Cc1ccccc1Cl]

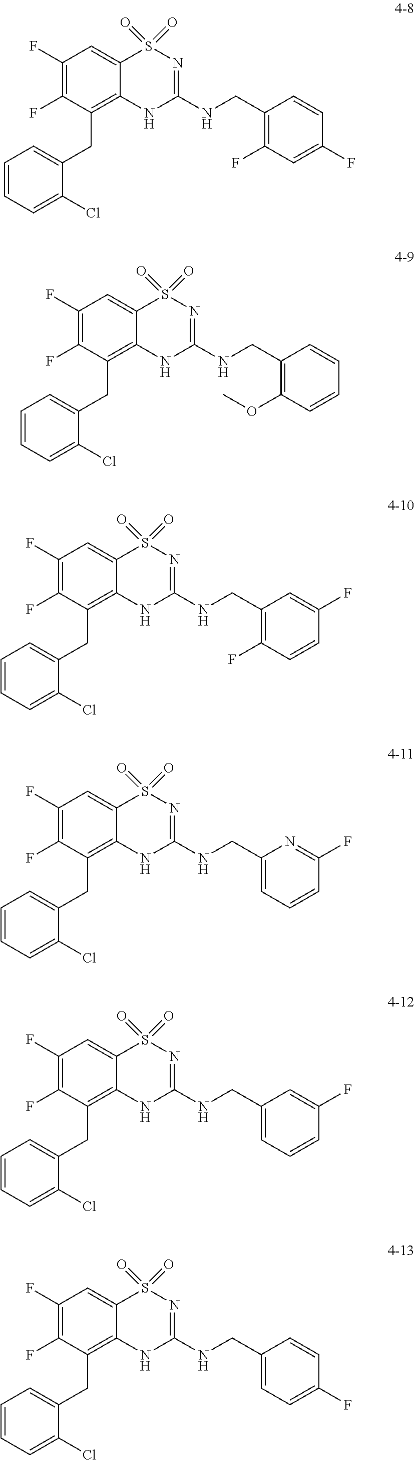 [SMILES: O=S1(=O)N=C(NCc2ccc(F)cc2F)Nc2c1cc(F)c(F)c2Cc1ccccc1Cl ; COc1ccccc1CNC1=NS(=O)(=O)c2cc(F)c(F)c(Cc3ccccc3Cl)c2N1 ; O=S1(=O)N=C(NCc2cc(F)ccc2F)Nc2c1cc(F)c(F)c2Cc1ccccc1Cl ; O=S1(=O)N=C(NCc2cccc(F)n2)Nc2c1cc(F)c(F)c2Cc1ccccc1Cl ; O=S1(=O)N=C(NCc2cccc(F)c2)Nc2c1cc(F)c(F)c2Cc1ccccc1Cl ; O=S1(=O)N=C(NCc2ccc(F)cc2)Nc2c1cc(F)c(F)c2Cc1ccccc1Cl]

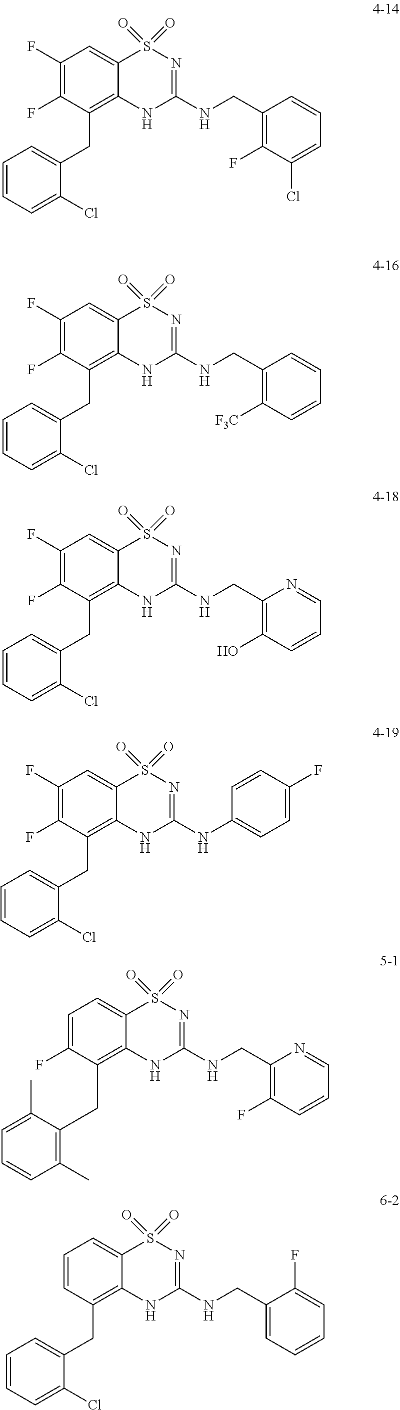 [SMILES: O=S1(=O)N=C(NCc2cccc(Cl)c2F)Nc2c1cc(F)c(F)c2Cc1ccccc1Cl ; O=S1(=O)N=C(NCc2ccccc2C(F)(F)F)Nc2c1cc(F)c(F)c2Cc1ccccc1Cl ; O=S1(=O)N=C(NCc2ncccc2O)Nc2c1cc(F)c(F)c2Cc1ccccc1Cl ; O=S1(=O)N=C(Nc2ccc(F)cc2)Nc2c1cc(F)c(F)c2Cc1ccccc1Cl ; Cc1cccc(C)c1Cc1c(F)ccc2c1NC(NCc1ncccc1F)=NS2(=O)=O ; O=S1(=O)N=C(NCc2ccccc2F)Nc2c(Cc3ccccc3Cl)cccc21]

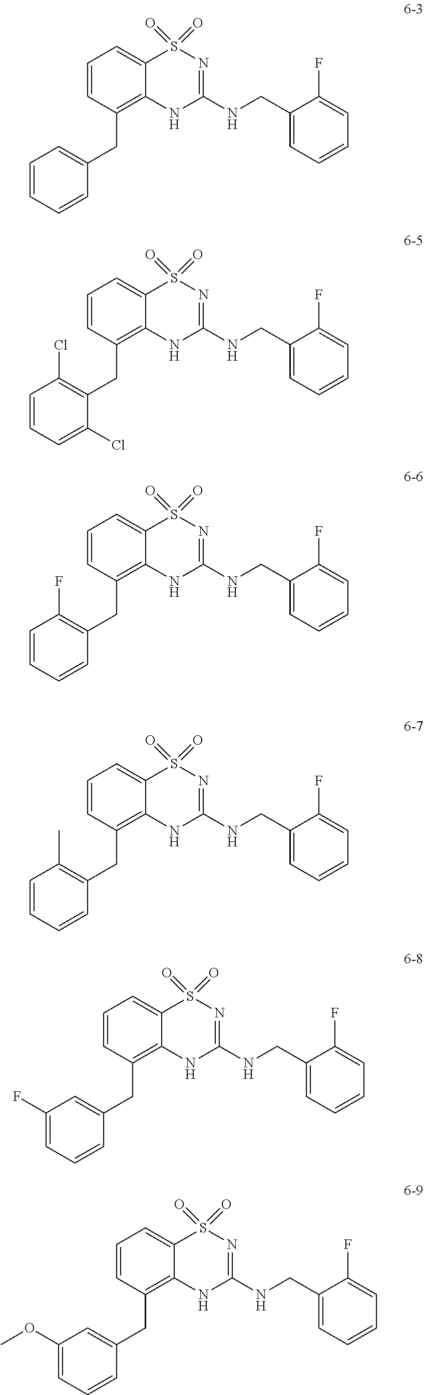 [SMILES: O=S1(=O)N=C(NCc2ccccc2F)Nc2c(Cc3ccccc3)cccc21 ; O=S1(=O)N=C(NCc2ccccc2F)Nc2c(Cc3c(Cl)cccc3Cl)cccc21 ; O=S1(=O)N=C(NCc2ccccc2F)Nc2c(Cc3ccccc3F)cccc21 ; Cc1ccccc1Cc1cccc2c1NC(NCc1ccccc1F)=NS2(=O)=O ; O=S1(=O)N=C(NCc2ccccc2F)Nc2c(Cc3cccc(F)c3)cccc21 ; COc1cccc(Cc2cccc3c2NC(NCc2ccccc2F)=NS3(=O)=O)c1]

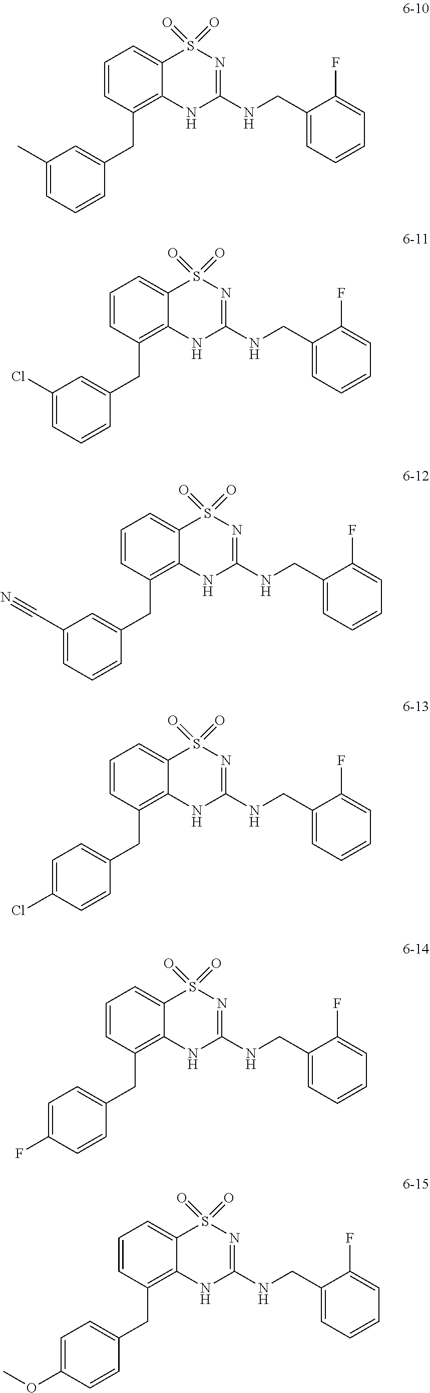 [SMILES: Cc1cccc(Cc2cccc3c2NC(NCc2ccccc2F)=NS3(=O)=O)c1 ; O=S1(=O)N=C(NCc2ccccc2F)Nc2c(Cc3cccc(Cl)c3)cccc21 ; N#Cc1cccc(Cc2cccc3c2NC(NCc2ccccc2F)=NS3(=O)=O)c1 ; O=S1(=O)N=C(NCc2ccccc2F)Nc2c(Cc3ccc(Cl)cc3)cccc21 ; O=S1(=O)N=C(NCc2ccccc2F)Nc2c(Cc3ccc(F)cc3)cccc21 ; COc1ccc(Cc2cccc3c2NC(NCc2ccccc2F)=NS3(=O)=O)cc1]

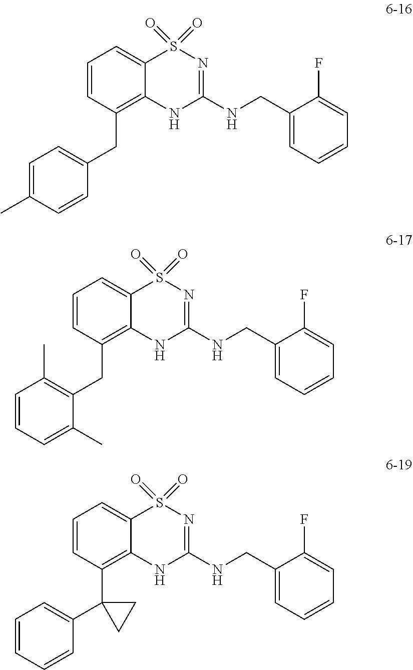 [SMILES: Cc1ccc(Cc2cccc3c2NC(NCc2ccccc2F)=NS3(=O)=O)cc1 ; Cc1cccc(C)c1Cc1cccc2c1NC(NCc1ccccc1F)=NS2(=O)=O ; O=S1(=O)N=C(NCc2ccccc2F)Nc2c(C3(c4ccccc4)CC3)cccc21]

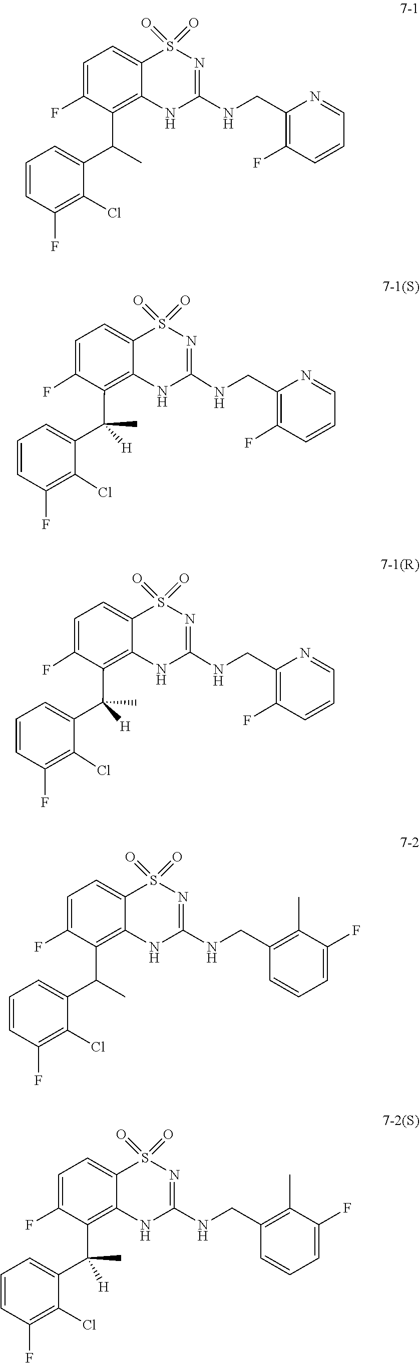 [SMILES: CC(c1cccc(F)c1Cl)c1c(F)ccc2c1NC(NCc1ncccc1F)=NS2(=O)=O ; C[C@@H](c1cccc(F)c1Cl)c1c(F)ccc2c1NC(NCc1ncccc1F)=NS2(=O)=O ; C[C@H](c1cccc(F)c1Cl)c1c(F)ccc2c1NC(NCc1ncccc1F)=NS2(=O)=O ; Cc1c(F)cccc1CNC1=NS(=O)(=O)c2ccc(F)c(C(C)c3cccc(F)c3Cl)c2N1 ; Cc1c(F)cccc1CNC1=NS(=O)(=O)c2ccc(F)c([C@@H](C)c3cccc(F)c3Cl)c2N1]

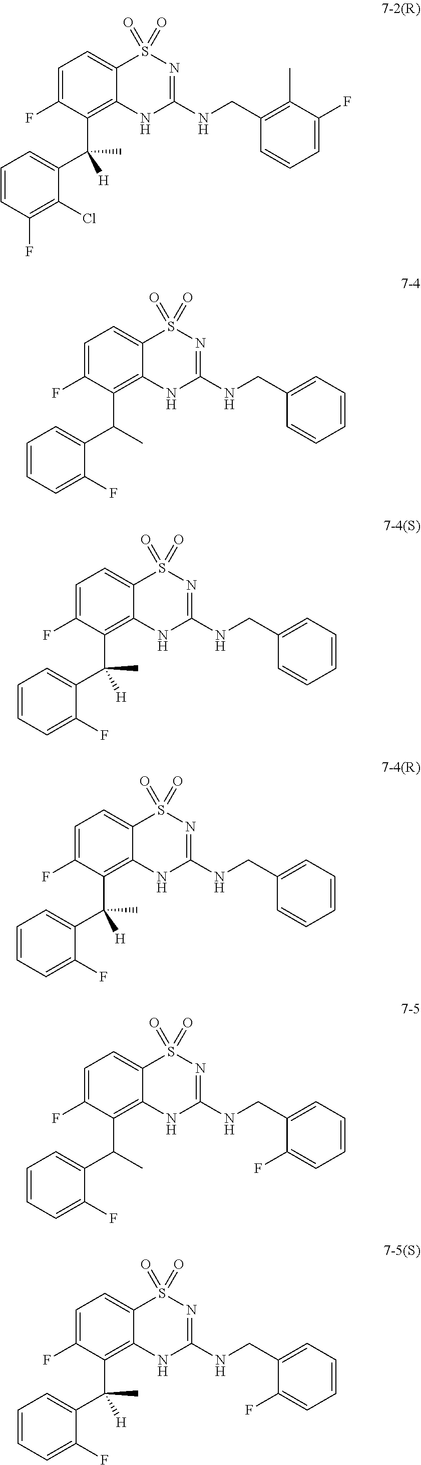 [SMILES: Cc1c(F)cccc1CNC1=NS(=O)(=O)c2ccc(F)c([C@H](C)c3cccc(F)c3Cl)c2N1 ; CC(c1ccccc1F)c1c(F)ccc2c1NC(NCc1ccccc1)=NS2(=O)=O ; C[C@@H](c1ccccc1F)c1c(F)ccc2c1NC(NCc1ccccc1)=NS2(=O)=O ; C[C@H](c1ccccc1F)c1c(F)ccc2c1NC(NCc1ccccc1)=NS2(=O)=O ; CC(c1ccccc1F)c1c(F)ccc2c1NC(NCc1ccccc1F)=NS2(=O)=O ; C[C@@H](c1ccccc1F)c1c(F)ccc2c1NC(NCc1ccccc1F)=NS2(=O)=O]

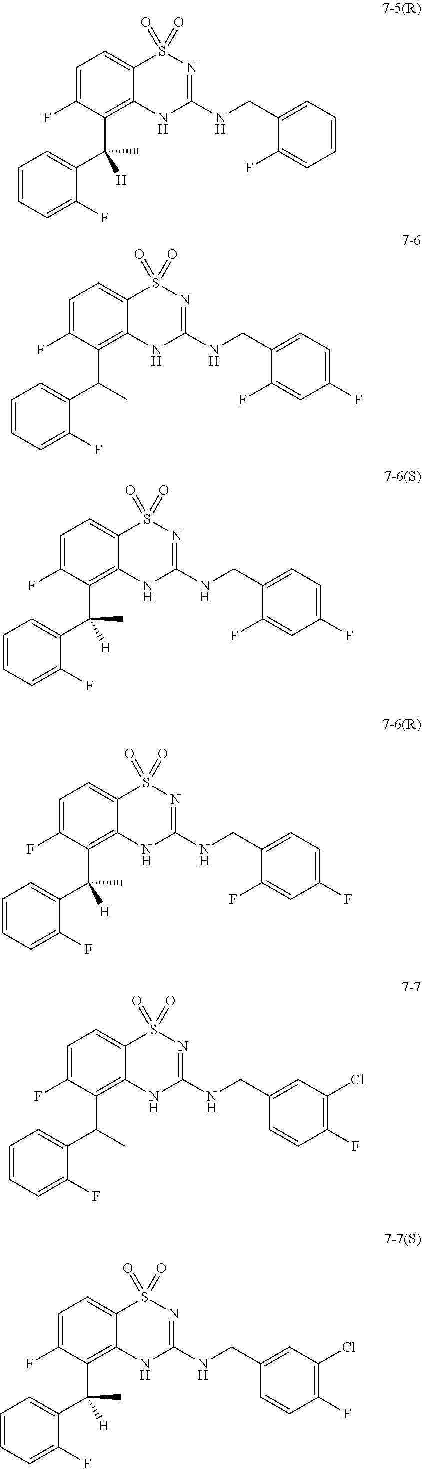 [SMILES: C[C@H](c1ccccc1F)c1c(F)ccc2c1NC(NCc1ccccc1F)=NS2(=O)=O ; CC(c1ccccc1F)c1c(F)ccc2c1NC(NCc1ccc(F)cc1F)=NS2(=O)=O ; C[C@@H](c1ccccc1F)c1c(F)ccc2c1NC(NCc1ccc(F)cc1F)=NS2(=O)=O ; C[C@H](c1ccccc1F)c1c(F)ccc2c1NC(NCc1ccc(F)cc1F)=NS2(=O)=O ; CC(c1ccccc1F)c1c(F)ccc2c1NC(NCc1ccc(F)c(Cl)c1)=NS2(=O)=O ; C[C@@H](c1ccccc1F)c1c(F)ccc2c1NC(NCc1ccc(F)c(Cl)c1)=NS2(=O)=O]

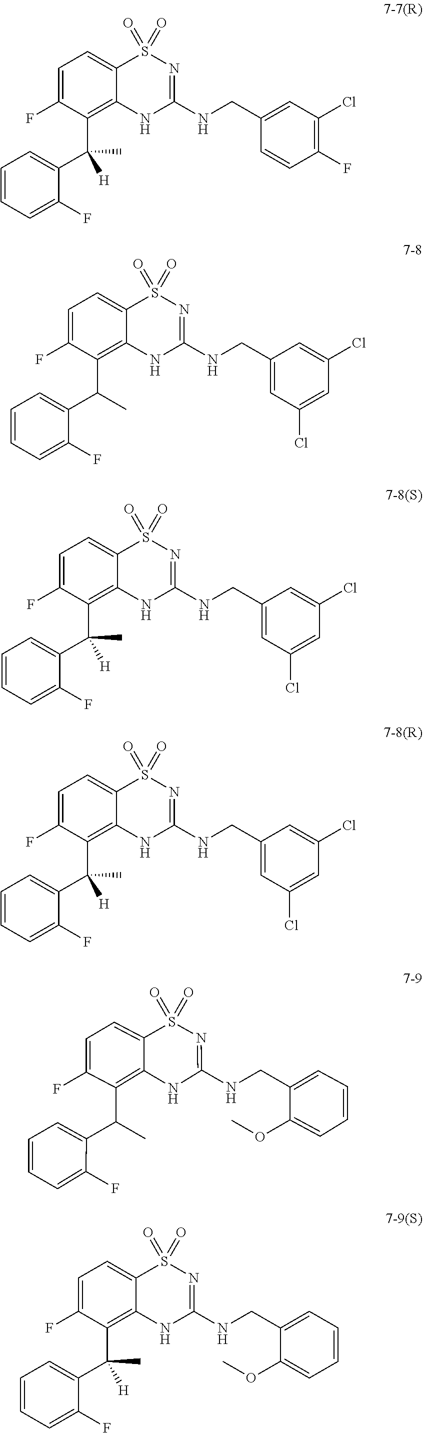 [SMILES: C[C@H](c1ccccc1F)c1c(F)ccc2c1NC(NCc1ccc(F)c(Cl)c1)=NS2(=O)=O ; CC(c1ccccc1F)c1c(F)ccc2c1NC(NCc1cc(Cl)cc(Cl)c1)=NS2(=O)=O ; C[C@@H](c1ccccc1F)c1c(F)ccc2c1NC(NCc1cc(Cl)cc(Cl)c1)=NS2(=O)=O ; C[C@H](c1ccccc1F)c1c(F)ccc2c1NC(NCc1cc(Cl)cc(Cl)c1)=NS2(=O)=O ; COc1ccccc1CNC1=NS(=O)(=O)c2ccc(F)c(C(C)c3ccccc3F)c2N1 ; COc1ccccc1CNC1=NS(=O)(=O)c2ccc(F)c([C@@H](C)c3ccccc3F)c2N1]

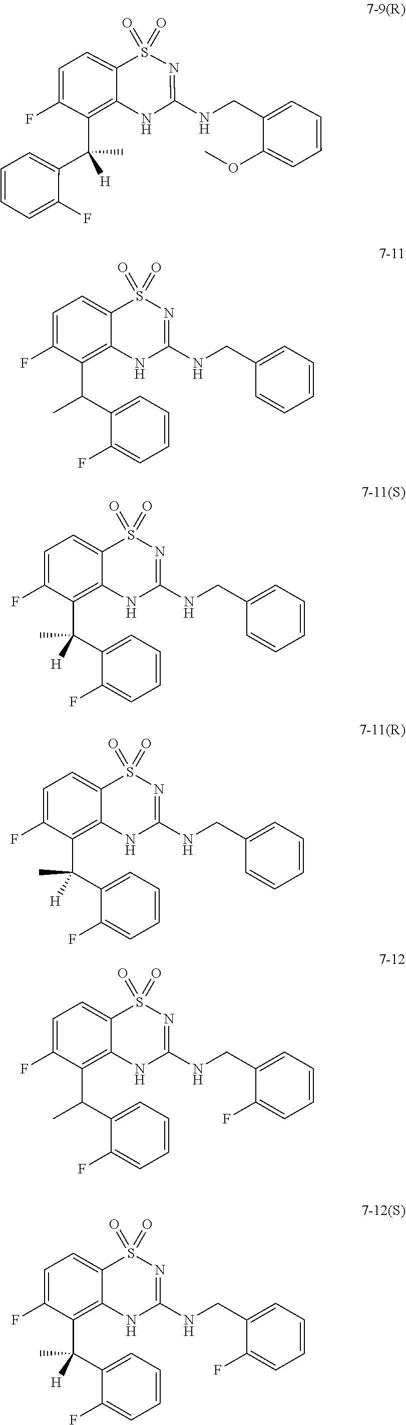

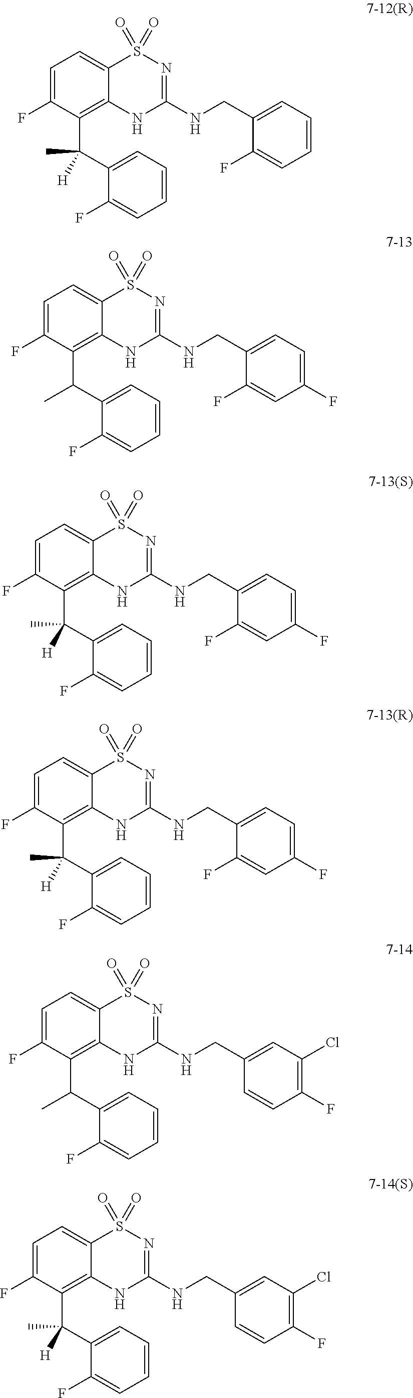

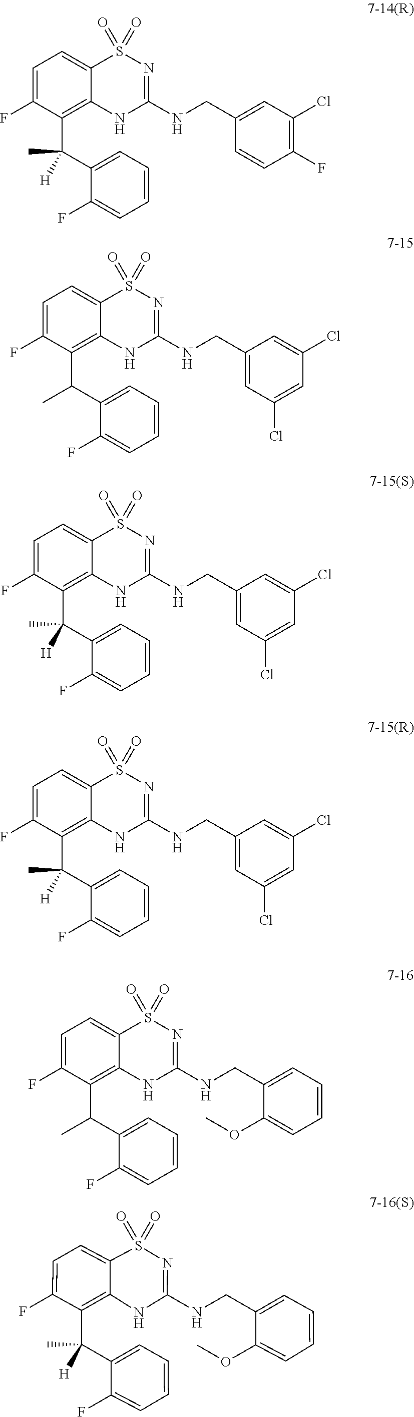

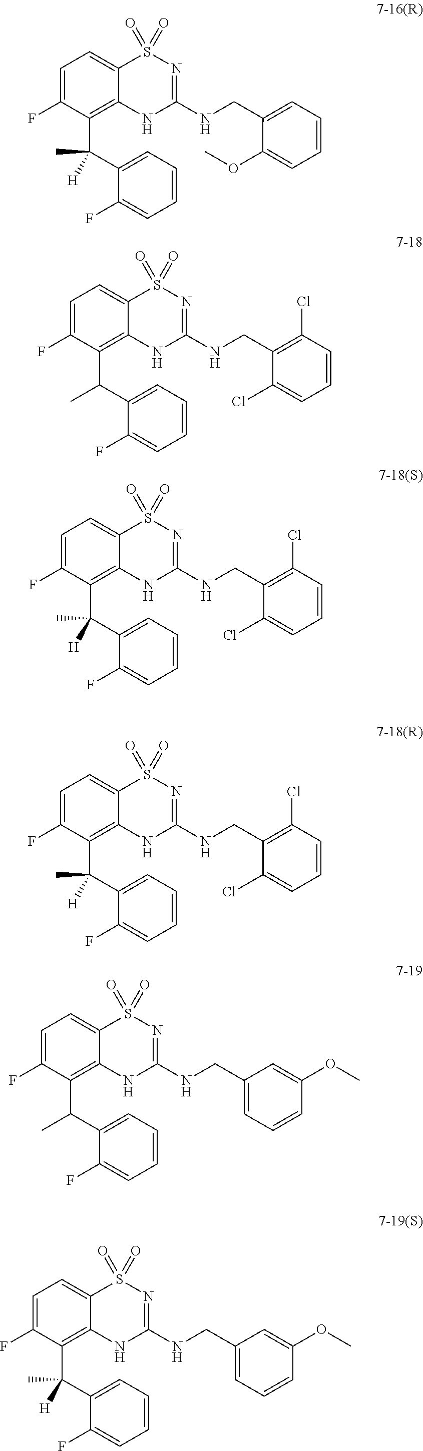

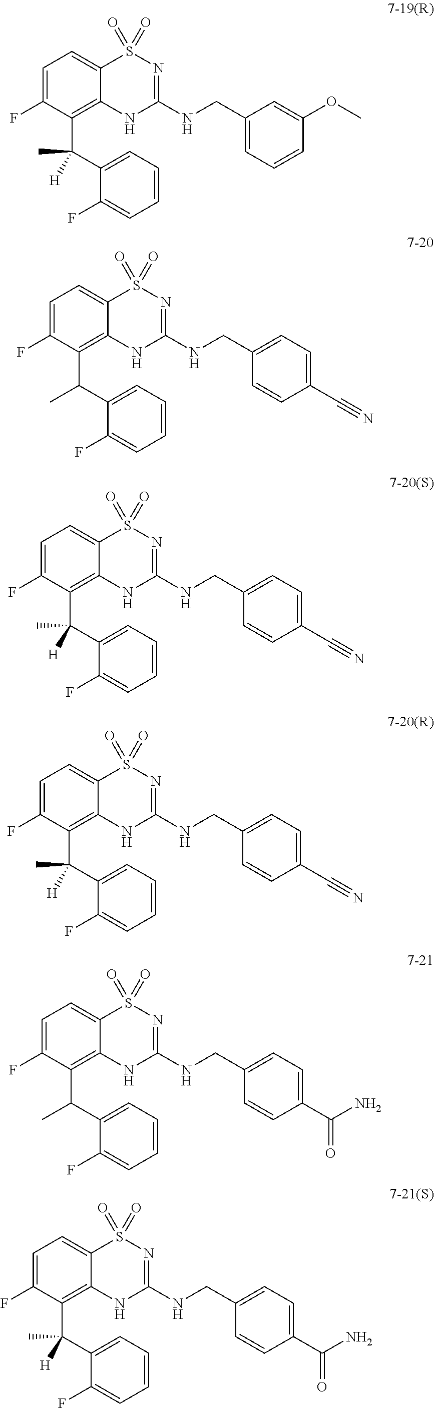

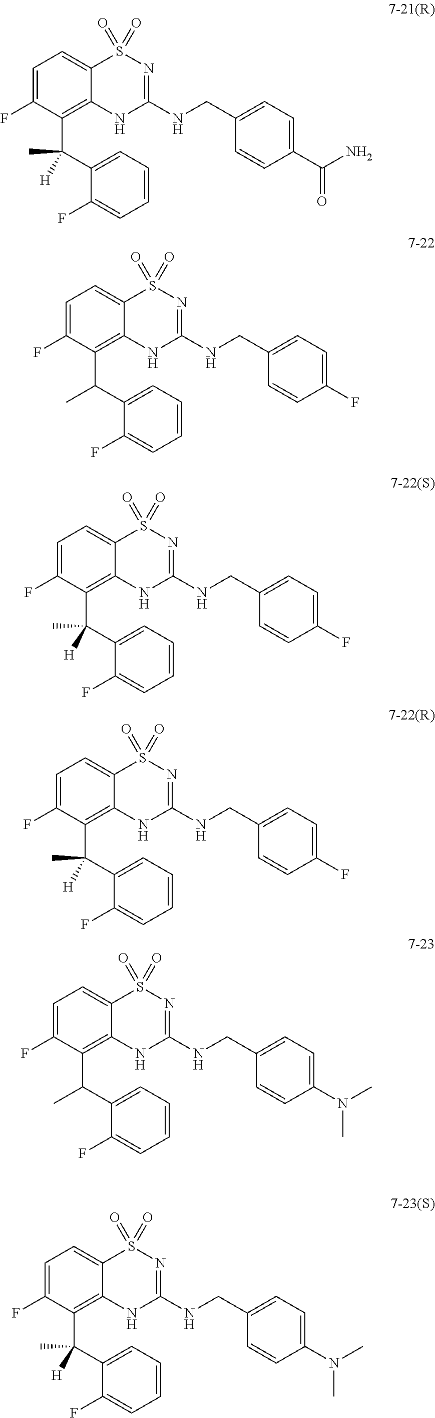

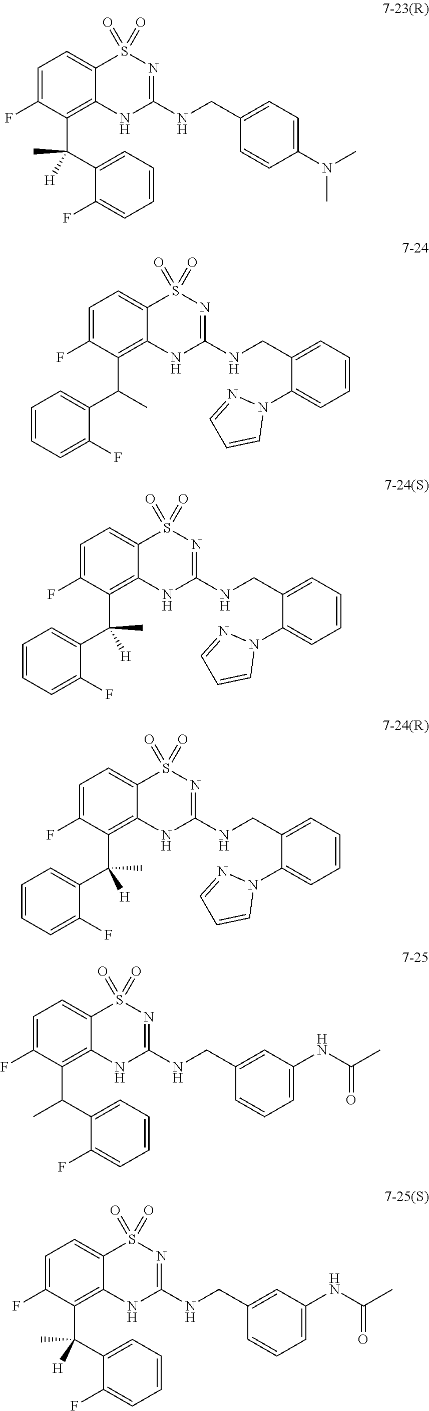

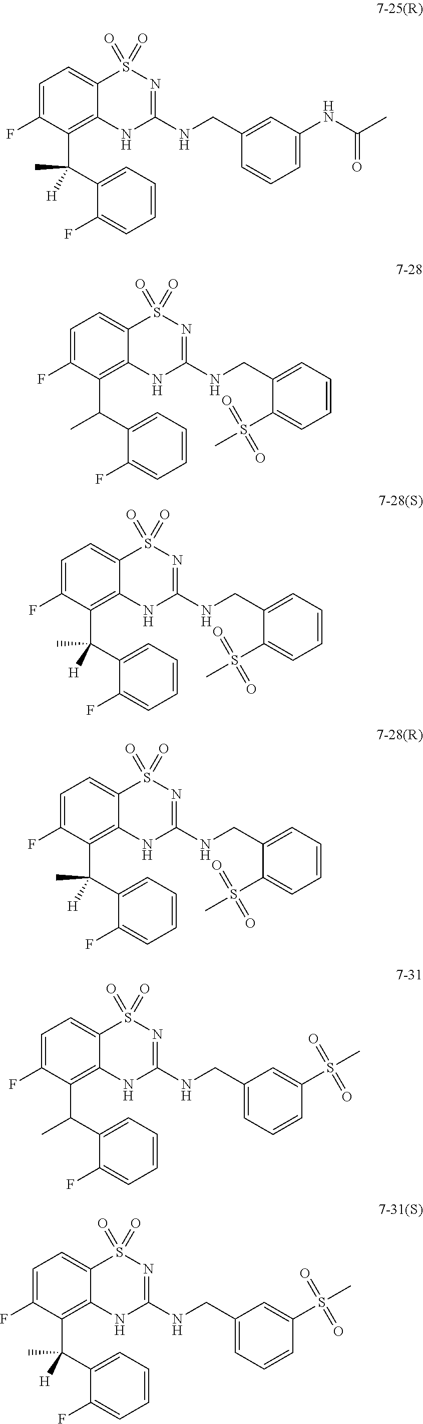

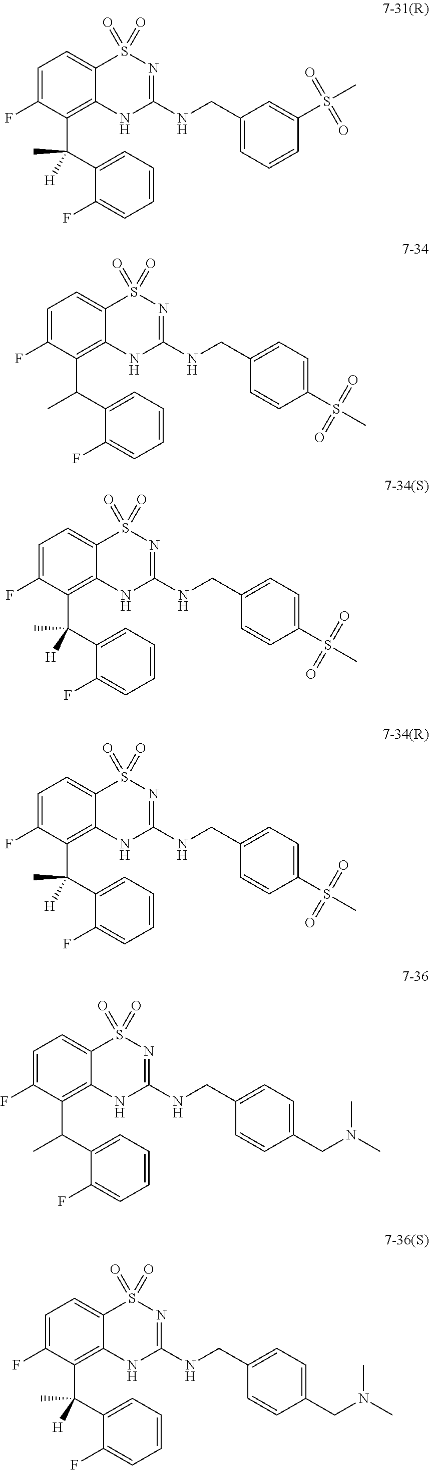

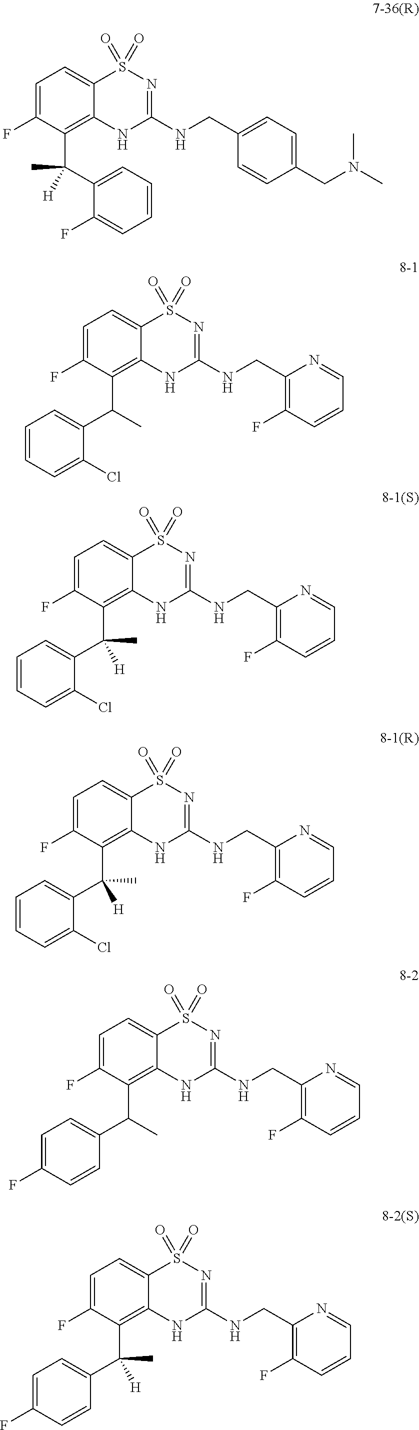 [SMILES: C[C@H](c1ccccc1F)c1c(F)ccc2c1NC(NCc1ccc(CN(C)C)cc1)=NS2(=O)=O ; CC(c1ccccc1Cl)c1c(F)ccc2c1NC(NCc1ncccc1F)=NS2(=O)=O ; C[C@@H](c1ccccc1Cl)c1c(F)ccc2c1NC(NCc1ncccc1F)=NS2(=O)=O ; C[C@H](c1ccccc1Cl)c1c(F)ccc2c1NC(NCc1ncccc1F)=NS2(=O)=O ; CC(c1ccc(F)cc1)c1c(F)ccc2c1NC(NCc1ncccc1F)=NS2(=O)=O ; C[C@@H](c1ccc(F)cc1)c1c(F)ccc2c1NC(NCc1ncccc1F)=NS2(=O)=O]

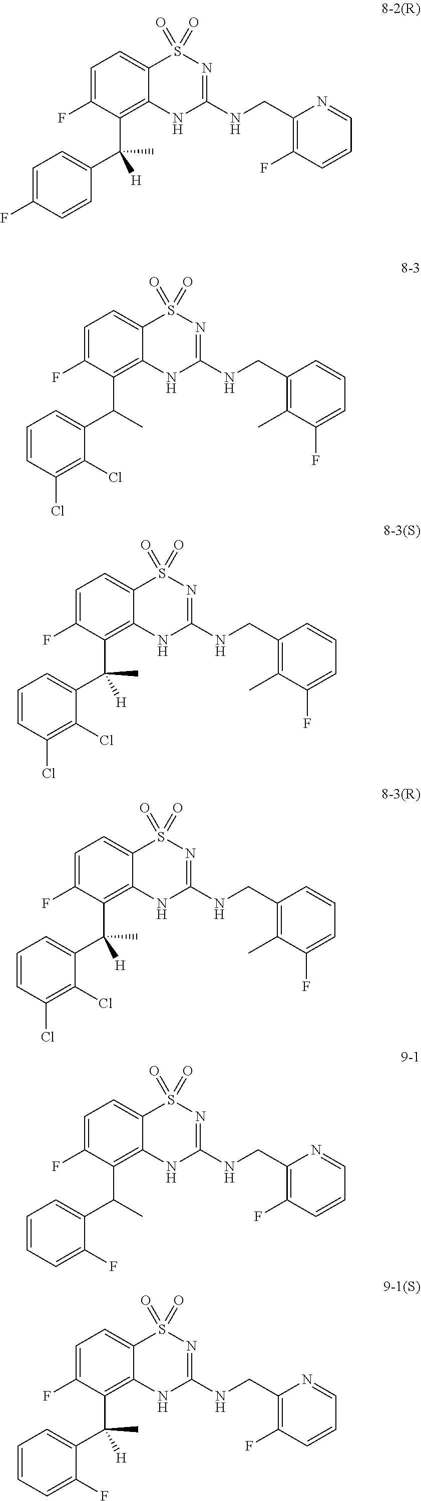 [SMILES: C[C@H](c1ccc(F)cc1)c1c(F)ccc2c1NC(NCc1ncccc1F)=NS2(=O)=O ; Cc1c(F)cccc1CNC1=NS(=O)(=O)c2ccc(F)c(C(C)c3cccc(Cl)c3Cl)c2N1 ; Cc1c(F)cccc1CNC1=NS(=O)(=O)c2ccc(F)c([C@@H](C)c3cccc(Cl)c3Cl)c2N1 ; Cc1c(F)cccc1CNC1=NS(=O)(=O)c2ccc(F)c([C@H](C)c3cccc(Cl)c3Cl)c2N1 ; CC(c1ccccc1F)c1c(F)ccc2c1NC(NCc1ncccc1F)=NS2(=O)=O ; C[C@@H](c1ccccc1F)c1c(F)ccc2c1NC(NCc1ncccc1F)=NS2(=O)=O]

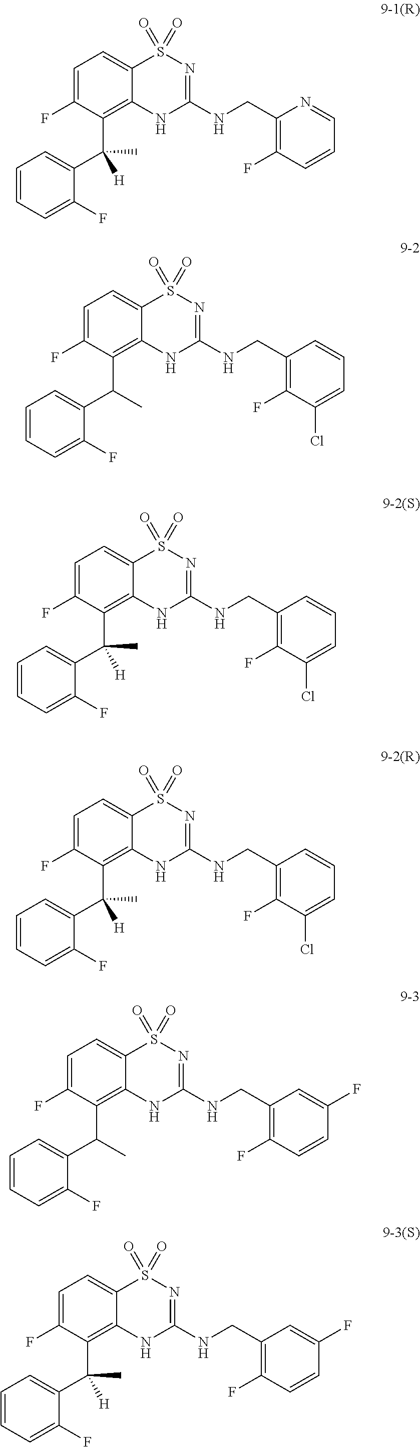 [SMILES: C[C@H](c1ccccc1F)c1c(F)ccc2c1NC(NCc1ncccc1F)=NS2(=O)=O ; CC(c1ccccc1F)c1c(F)ccc2c1NC(NCc1cccc(Cl)c1F)=NS2(=O)=O ; C[C@@H](c1ccccc1F)c1c(F)ccc2c1NC(NCc1cccc(Cl)c1F)=NS2(=O)=O ; C[C@H](c1ccccc1F)c1c(F)ccc2c1NC(NCc1cccc(Cl)c1F)=NS2(=O)=O ; CC(c1ccccc1F)c1c(F)ccc2c1NC(NCc1cc(F)ccc1F)=NS2(=O)=O ; C[C@@H](c1ccccc1F)c1c(F)ccc2c1NC(NCc1cc(F)ccc1F)=NS2(=O)=O]

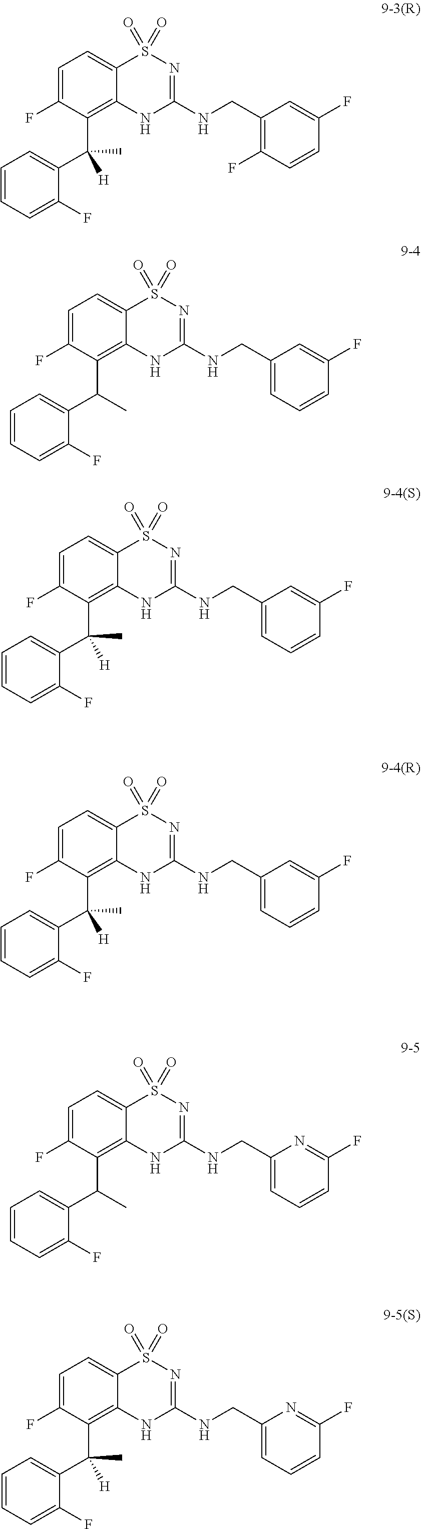 [SMILES: C[C@H](c1ccccc1F)c1c(F)ccc2c1NC(NCc1cc(F)ccc1F)=NS2(=O)=O ; CC(c1ccccc1F)c1c(F)ccc2c1NC(NCc1cccc(F)c1)=NS2(=O)=O ; C[C@@H](c1ccccc1F)c1c(F)ccc2c1NC(NCc1cccc(F)c1)=NS2(=O)=O ; C[C@H](c1ccccc1F)c1c(F)ccc2c1NC(NCc1cccc(F)c1)=NS2(=O)=O ; CC(c1ccccc1F)c1c(F)ccc2c1NC(NCc1cccc(F)n1)=NS2(=O)=O ; C[C@@H](c1ccccc1F)c1c(F)ccc2c1NC(NCc1cccc(F)n1)=NS2(=O)=O]

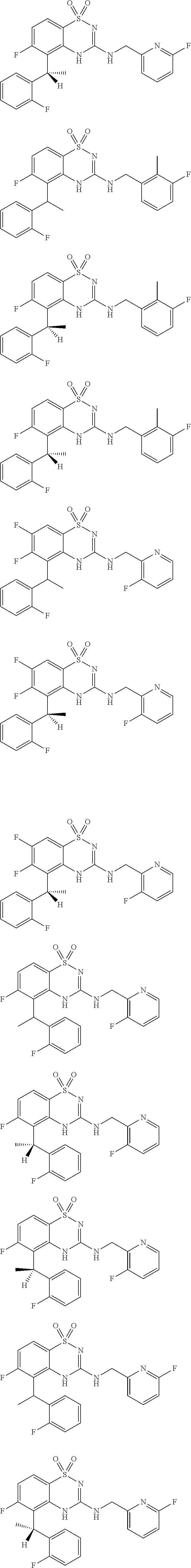

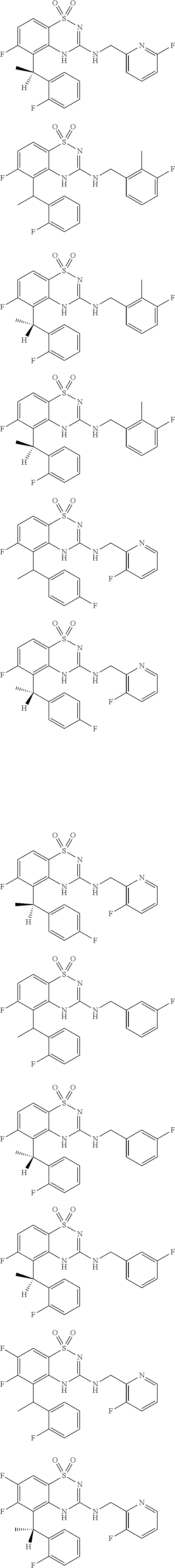

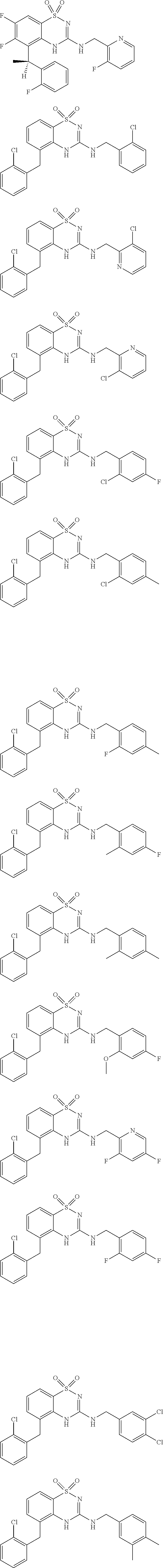

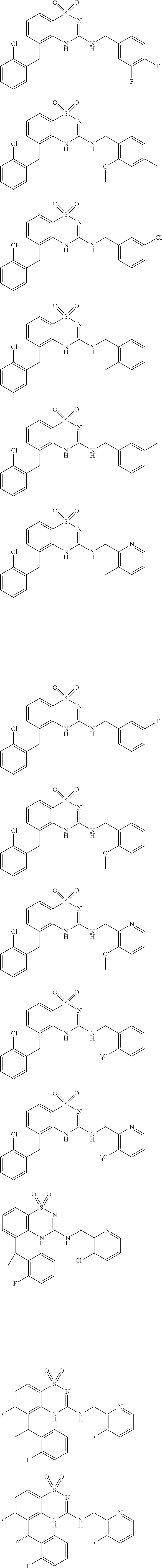

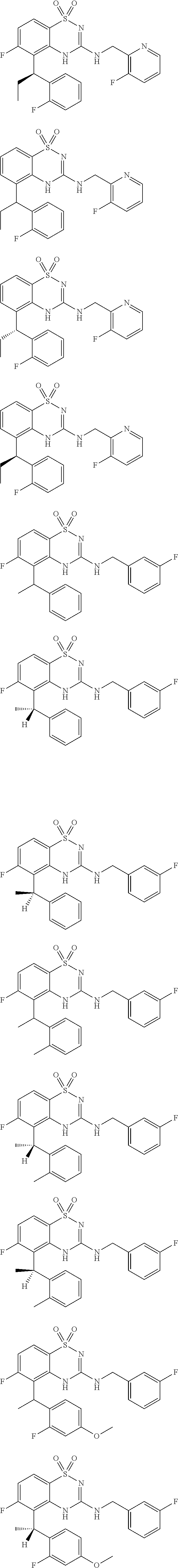

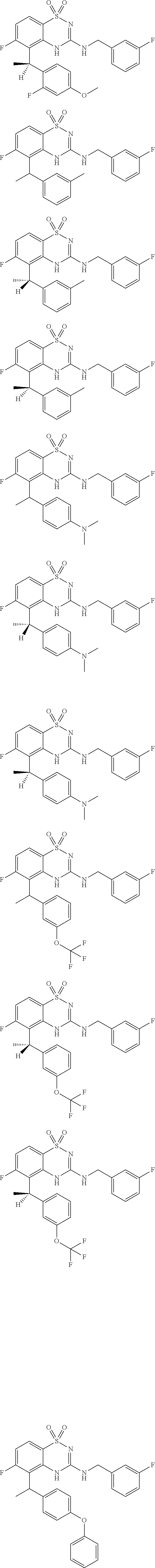

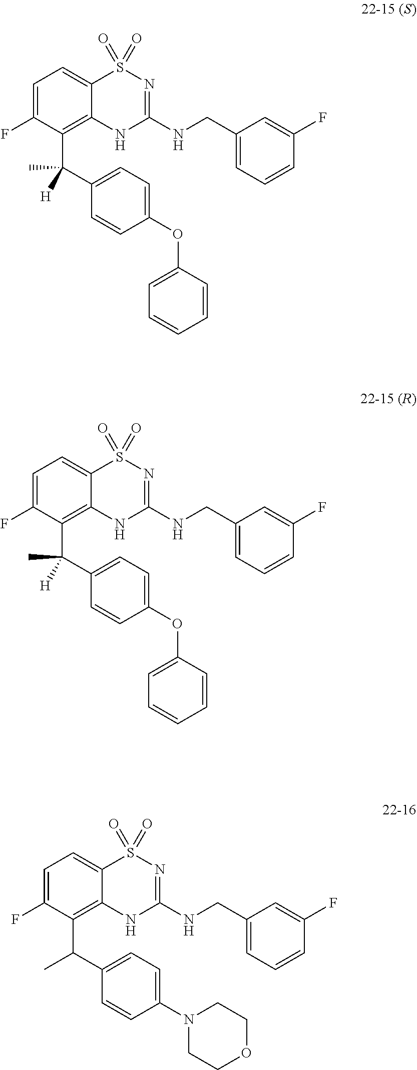 [SMILES: C[C@@H](c1ccc(Oc2ccccc2)cc1)c1c(F)ccc2c1NC(NCc1cccc(F)c1)=NS2(=O)=O ; C[C@H](c1ccc(Oc2ccccc2)cc1)c1c(F)ccc2c1NC(NCc1cccc(F)c1)=NS2(=O)=O ; CC(c1ccc(N2CCOCC2)cc1)c1c(F)ccc2c1NC(NCc1cccc(F)c1)=NS2(=O)=O]

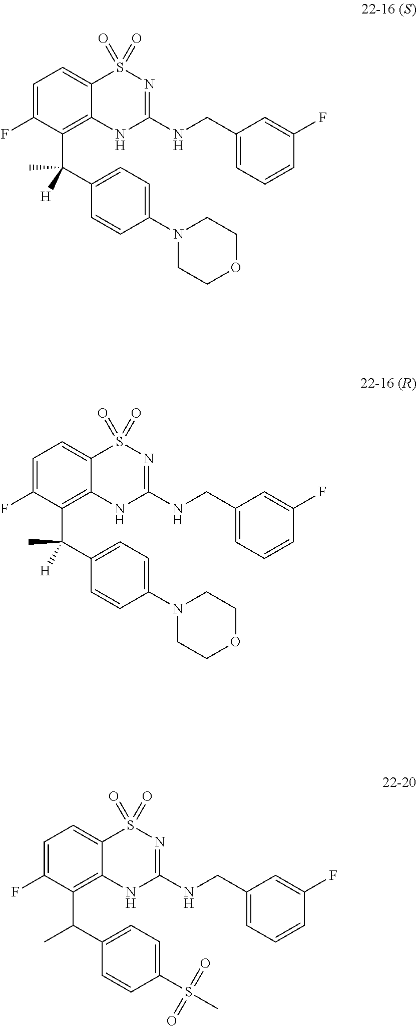 [SMILES: C[C@@H](c1ccc(N2CCOCC2)cc1)c1c(F)ccc2c1NC(NCc1cccc(F)c1)=NS2(=O)=O ; C[C@H](c1ccc(N2CCOCC2)cc1)c1c(F)ccc2c1NC(NCc1cccc(F)c1)=NS2(=O)=O ; CC(c1ccc(S(C)(=O)=O)cc1)c1c(F)ccc2c1NC(NCc1cccc(F)c1)=NS2(=O)=O]

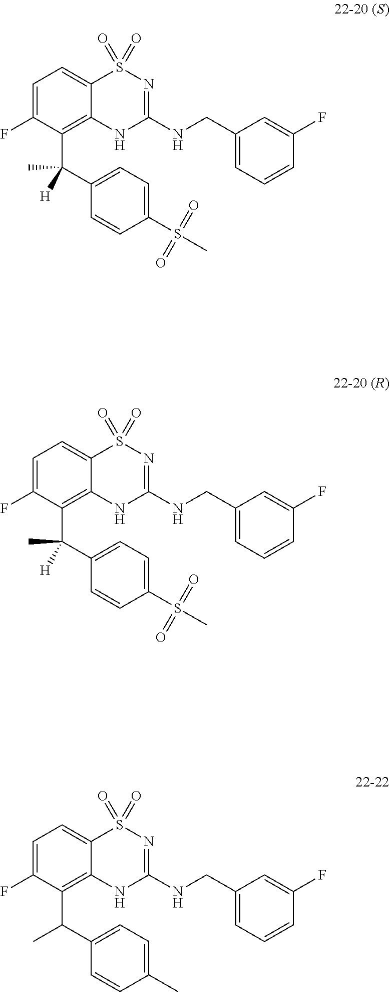 [SMILES: C[C@@H](c1ccc(S(C)(=O)=O)cc1)c1c(F)ccc2c1NC(NCc1cccc(F)c1)=NS2(=O)=O ; C[C@H](c1ccc(S(C)(=O)=O)cc1)c1c(F)ccc2c1NC(NCc1cccc(F)c1)=NS2(=O)=O ; Cc1ccc(C(C)c2c(F)ccc3c2NC(NCc2cccc(F)c2)=NS3(=O)=O)cc1]

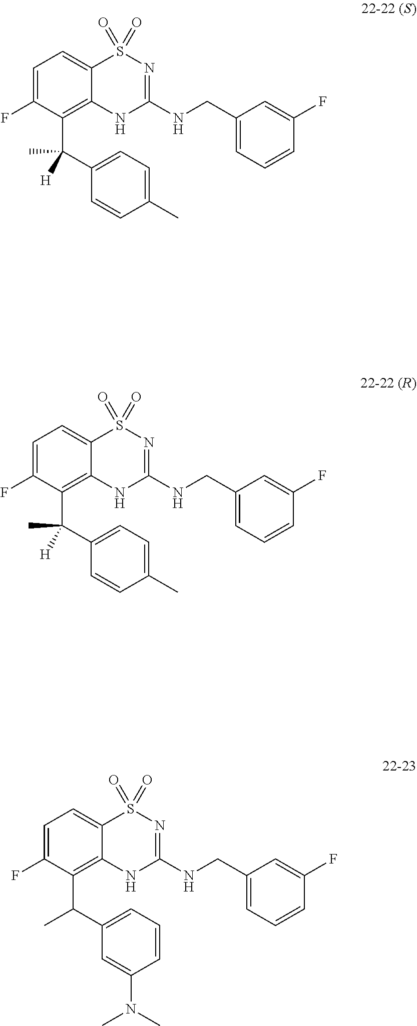 [SMILES: Cc1ccc([C@H](C)c2c(F)ccc3c2NC(NCc2cccc(F)c2)=NS3(=O)=O)cc1 ; Cc1ccc([C@@H](C)c2c(F)ccc3c2NC(NCc2cccc(F)c2)=NS3(=O)=O)cc1 ; CC(c1cccc(N(C)C)c1)c1c(F)ccc2c1NC(NCc1cccc(F)c1)=NS2(=O)=O]

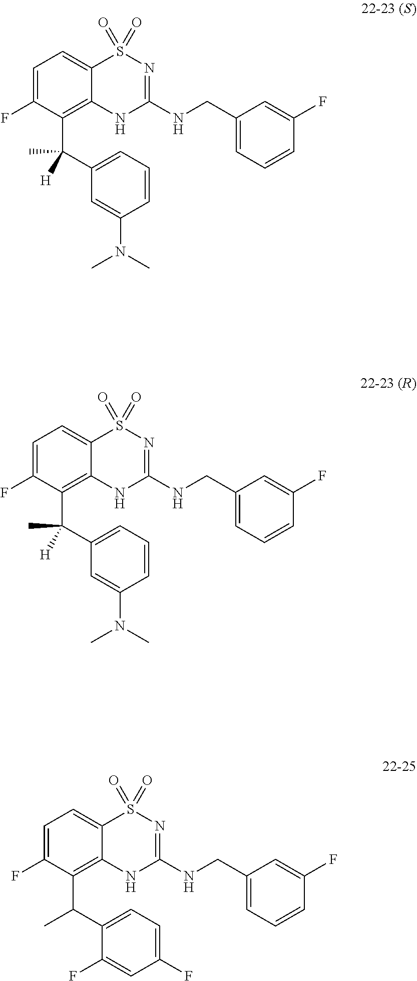 [SMILES: C[C@@H](c1cccc(N(C)C)c1)c1c(F)ccc2c1NC(NCc1cccc(F)c1)=NS2(=O)=O ; C[C@H](c1cccc(N(C)C)c1)c1c(F)ccc2c1NC(NCc1cccc(F)c1)=NS2(=O)=O ; CC(c1ccc(F)cc1F)c1c(F)ccc2c1NC(NCc1cccc(F)c1)=NS2(=O)=O]

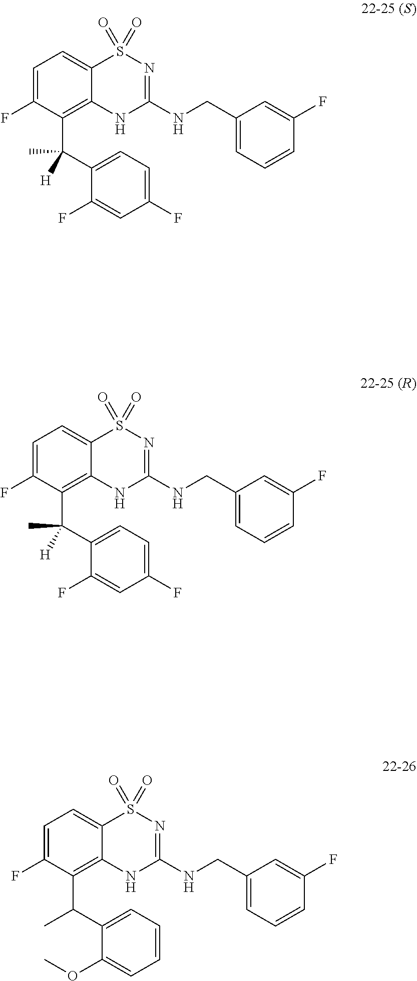 [SMILES: C[C@@H](c1ccc(F)cc1F)c1c(F)ccc2c1NC(NCc1cccc(F)c1)=NS2(=O)=O ; C[C@H](c1ccc(F)cc1F)c1c(F)ccc2c1NC(NCc1cccc(F)c1)=NS2(=O)=O ; COc1ccccc1C(C)c1c(F)ccc2c1NC(NCc1cccc(F)c1)=NS2(=O)=O]

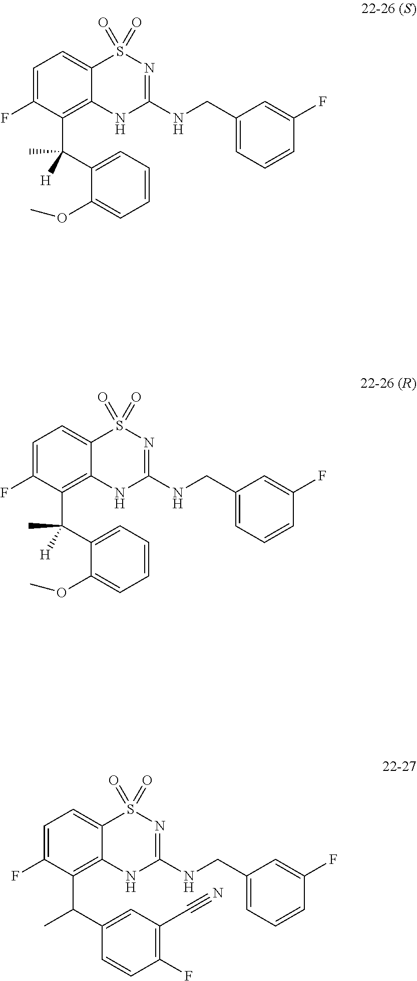 [SMILES: COc1ccccc1[C@H](C)c1c(F)ccc2c1NC(NCc1cccc(F)c1)=NS2(=O)=O ; COc1ccccc1[C@@H](C)c1c(F)ccc2c1NC(NCc1cccc(F)c1)=NS2(=O)=O ; CC(c1ccc(F)c(C#N)c1)c1c(F)ccc2c1NC(NCc1cccc(F)c1)=NS2(=O)=O]

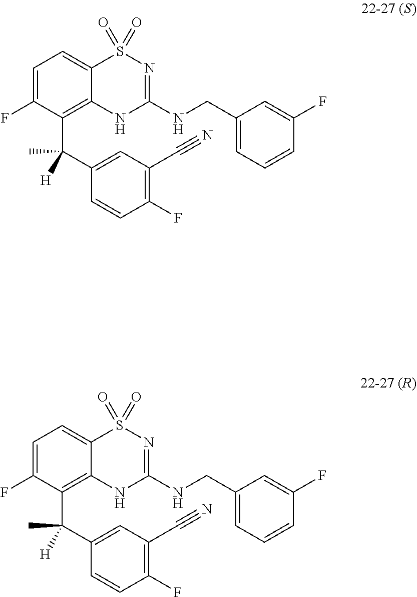 [SMILES: C[C@@H](c1ccc(F)c(C#N)c1)c1c(F)ccc2c1NC(NCc1cccc(F)c1)=NS2(=O)=O ; C[C@H](c1ccc(F)c(C#N)c1)c1c(F)ccc2c1NC(NCc1cccc(F)c1)=NS2(=O)=O]

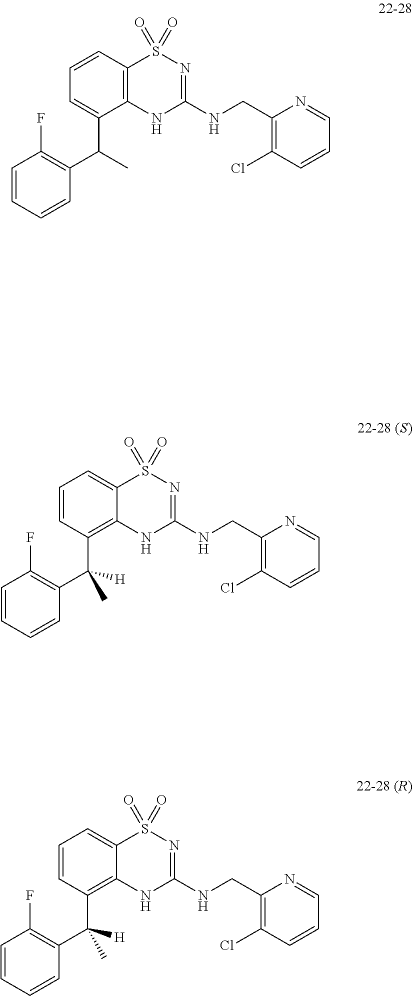 [SMILES: CC(c1ccccc1F)c1cccc2c1NC(NCc1ncccc1Cl)=NS2(=O)=O ; C[C@@H](c1ccccc1F)c1cccc2c1NC(NCc1ncccc1Cl)=NS2(=O)=O ; C[C@H](c1ccccc1F)c1cccc2c1NC(NCc1ncccc1Cl)=NS2(=O)=O]

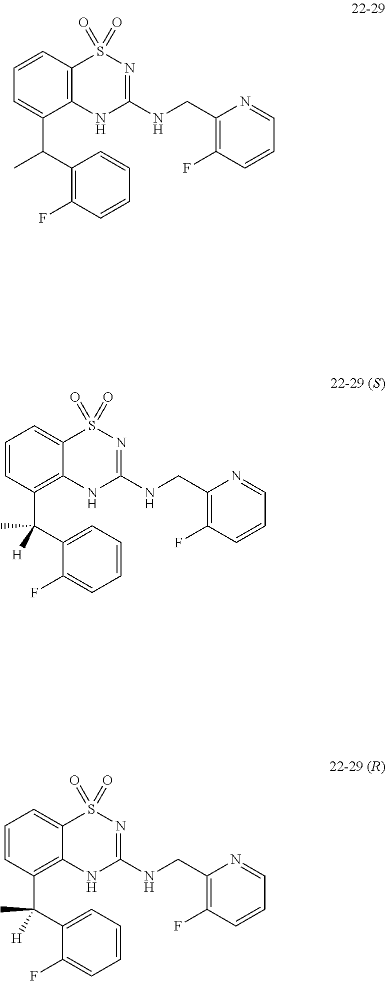 [SMILES: CC(c1ccccc1F)c1cccc2c1NC(NCc1ncccc1F)=NS2(=O)=O ; C[C@@H](c1ccccc1F)c1cccc2c1NC(NCc1ncccc1F)=NS2(=O)=O ; C[C@H](c1ccccc1F)c1cccc2c1NC(NCc1ncccc1F)=NS2(=O)=O]

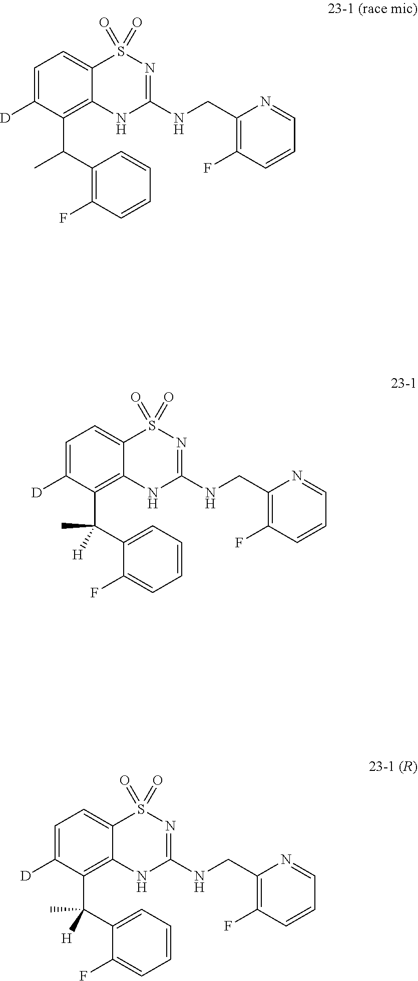 [SMILES: [2H]c1ccc2c(c1C(C)c1ccccc1F)NC(NCc1ncccc1F)=NS2(=O)=O ; [2H]c1ccc2c(c1[C@H](C)c1ccccc1F)NC(NCc1ncccc1F)=NS2(=O)=O ; [2H]c1ccc2c(c1[C@@H](C)c1ccccc1F)NC(NCc1ncccc1F)=NS2(=O)=O]

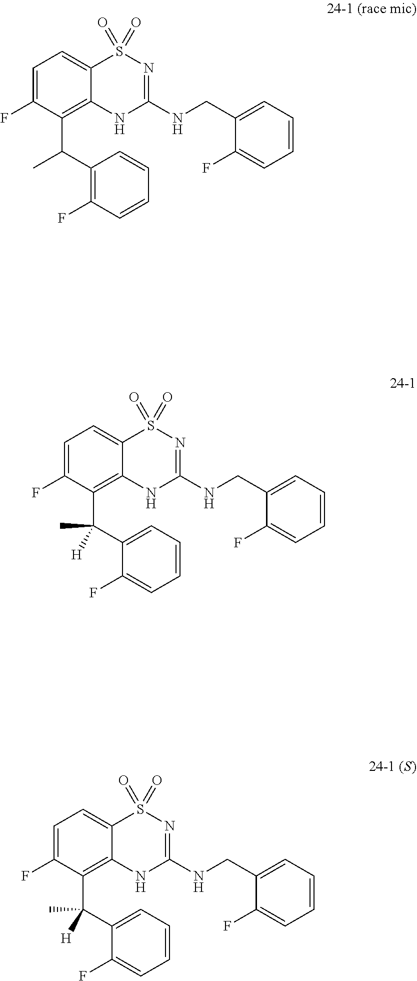 [SMILES: CC(c1ccccc1F)c1c(F)ccc2c1NC(NCc1ccccc1F)=NS2(=O)=O ; C[C@H](c1ccccc1F)c1c(F)ccc2c1NC(NCc1ccccc1F)=NS2(=O)=O ; C[C@@H](c1ccccc1F)c1c(F)ccc2c1NC(NCc1ccccc1F)=NS2(=O)=O]

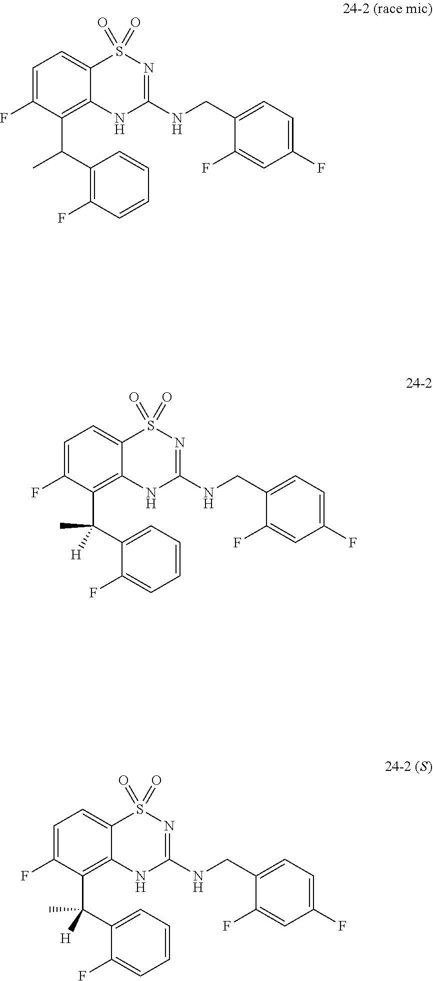 [SMILES: CC(c1ccccc1F)c1c(F)ccc2c1NC(NCc1ccc(F)cc1F)=NS2(=O)=O ; C[C@H](c1ccccc1F)c1c(F)ccc2c1NC(NCc1ccc(F)cc1F)=NS2(=O)=O ; C[C@@H](c1ccccc1F)c1c(F)ccc2c1NC(NCc1ccc(F)cc1F)=NS2(=O)=O]

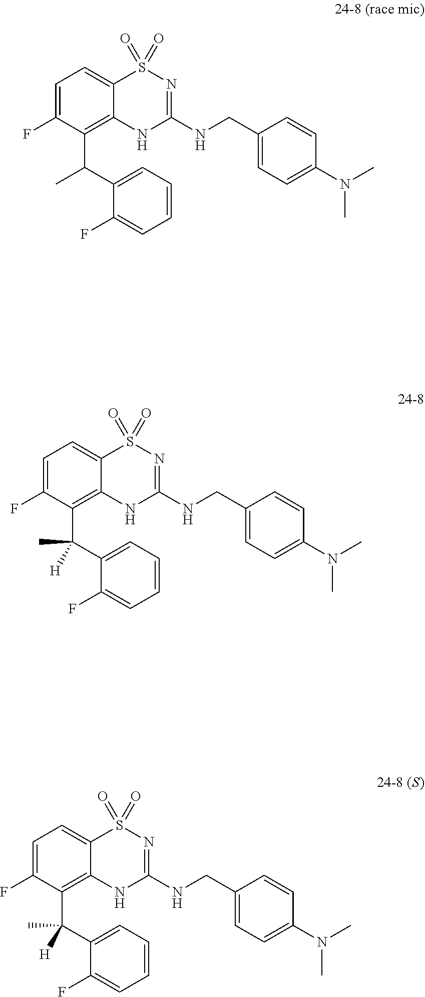 [SMILES: CC(c1ccccc1F)c1c(F)ccc2c1NC(NCc1ccc(N(C)C)cc1)=NS2(=O)=O ; C[C@H](c1ccccc1F)c1c(F)ccc2c1NC(NCc1ccc(N(C)C)cc1)=NS2(=O)=O ; C[C@@H](c1ccccc1F)c1c(F)ccc2c1NC(NCc1ccc(N(C)C)cc1)=NS2(=O)=O]

143-144. (canceled)

145. A pharmaceutical composition comprising the compound of claim 74, a pharmaceutically acceptable isomer, racemate, hydrate, solvate, or salt thereof and at least one pharmaceutically acceptable excipient.

146. A method of modulating a Mas-Related G-Protein Receptor (MRGPR) X2 or a MRGPRX2 ortholog by contacting MRGPRX2 or MRGPRX2 ortholog with an effective amount of the pharmaceutical composition of claim 145.

147. A method of treating a MRGPRX2 or a MRGPRX2 ortholog dependent condition by administering to a subject in need thereof an effective amount of the pharmaceutical composition of claim 145.

148. A method of treating a pseudo-allergic reaction, an itch associated condition, a pain associated condition, a cancer associated condition, an inflammatory or autoimmune disorder by administering to a subject in need thereof an effective amount of the pharmaceutical composition of claim 145.

149-150. (canceled)

151. The method of claim 148, wherein the itch associated condition is urticaria, pruritus, atopic dermatitis, dry skin, psoriasis, contact dermatitis, or eczema.

152-154. (canceled)
